SEQUENCE LISTING (107 sequences)

SEQ ID NO: 1 - protein, 1180 aa, Saccharomyces cerevisiae
MSSSKKLAGLRDNFSLLGEKNKILVANRGEIPIRIFRSAHELSMRTIAIYSHEDRLSMHRLKADEAYVIGEEGQYTPVGAYLAMDEIIEIAKKHKVDFIHPGYGFLSENSEFADKVVKAGITWIGPPAEVIDSVGDKVSARHLAARANVPTVPGTPGPIETVQEALDFVNEYGYPVIIKAAFGGGGRGMRVVREGDDVADAFQRATSEARTAFGNGTCFVERFLDKPKHIEVQLLADNHGNVVHLFERDCSVQRRHQKVVEVAPAKTLPREVRDAILTDAVKLAKVCGYRNAGTAEFLVDNQNRHYFIEINPRIQVEHTITEEITGIDIVSAQIQIAAGATLTQLGLLQDKITTRGFSIQCRITTEDPSKNFQPDTGRLEVYRSAGGNGVRLDGGNAYAGATISPHYDSMLVKCSCSGSTYEIVRRKMIRALIEFRIRGVKTNIPFLLTLLTNPVFIEGTYWTTFIDDTPQLFQMVSSQNRAQKLLHYLADLAVNGSSIKGQIGLPKLKSNPSVPHLHDAQGNVINVTKSAPPSGWRQVLLEKGPSEFAKQVRQFNGTLLMDTTWRDAHQSLLATRVRTHDLATIAPTTAHALAGAFALECWGGATFDVAMRFLHEDPWERLRKLRSLVPNIPFQMLLRGANGVAYSSLPDNAIDHFVKQAKDNGVDIFRVFDALNDLEQLKVGVNAVKKAGGVVEATVCYSGDMLQPGKKYNLDYYLEVVEKIVQMGTHILGIKDMAGTMKPAAAKLLIGSLRTRYPDLPIHVHSHDSAGTAVASMTACALAGADVVDVAINSMSGLTSQPSINALLASLEGNIDTGINVEHVRELDAYWAEMRLLYSCFEADLKGPDPEVYQHEIPGGQLTNLLFQAQQLGLGEQWAETKRAYREANYLLGDIVKVTPTSKVVGDLAQFMVSNKLTSDDIRRLANSLDFPDSVMDFFEGLIGQPYGGFPEPLRSDVLRNKRRKLTCRPGLELEPFDLEKIREDLQNRFGDIDECDVASYNMYPRVYEDFQKIRETYGDLSVLPTKNFLAPAEPDEEIEVTIEQGKTLIIKLQAVGDLNKKTGQREVYFELNGELRKIRVADKSQNIQSVAKPKADVHDTHQIGAPMAGVIIEVKVHKGSLVKKGESIAVLSAMKMEMVVSSPADGQVKDVFIKDGESVDASDLLVVLEEETLPPSQKK

SEQ ID NO: 2 - protein, 340 aa, Saccharomyces cerevisiae
MVKVAILGASGGVGQPLSLLLKLSPYVSELALYDIRAAEGIGKDLSHINTNSSCVGYDKDSIENTLSNAQVVLIPAGVPRKPGLTRDDLFKMNAGIVKSLVTAVGKFAPNARILVISNPVNSLVPIAVETLKKMGKFKPGNVMGVTNLDLVRAETFLVDYLMLKNPKIGQEQDKTTMHRKVTVIGGHSGETIIPIITDKSLVFQLDKQYEHFIHRVQFGGDEIVKAKQGAGSATLSMAFAGAKFAEEVLRSFHNEKPETESLSAFVYLPGLKNGKKAQQLVGDNSIEYFSLPIVLRNGSVVSIDTSVLEKLSPREEQLVNTAVKELRKNIEKGKSFILDS

SEQ ID NO: 3 - protein, 438 aa, Schizosaccharomyces pombe
MGELKEILKQRYHELLDWNVKAPHVPLSQRLKHFTWSWFACTMATGGVGLIIGSFPFRFYGLNTIGKIVYILQIFLFSLFGSCMLFRFIKYPSTIKDSWNHHLEKLFIATCLLSISTFIDMLAIYAYPDTGEWMVWVIRILYYIYVAVSFIYCVMAFFTIFNNHVYTIETASPAWILPIFPPMICGVIAGAVNSTQPAHQLKNMVIFGILFQGLGFWVYLLLFAVNVLRFFTVGLAKPQDRPGMFMFVGPPAFSGLALINIARGAMGSRPYIFVGANSSEYLGFVSTFMAIFIWGLAAWCYCLAMVSFLAGFFTRAPLKFACGWFAFIFPNVGFVNCTIEIGKMIDSKAFQMFGHIIGVILCIQWILLMYLMVRAFLVNDLCYPGKDEDAHPPPKPNTGVLNPTFPPEKAPASLEKVDTHVTSTGGESDPPSSEHESV

SEQ ID NO: 4 - DNA, 4144 bp, Saccharomyces cerevisiae
tcgagctatttattataagagtcagaattggcgcagggagtgttaagtaagaagtactccccatcggatatttcctattgtgtttctgtgatttttagtccttttttcttttctcttactttcggtatccttacttgattacatacataaacaagcccctcttttcttccaaactcttgtagcttactatctgtggcccgtcatttgagtttgatttttttgccaattactatattgcaaaataaaggacagttactaggagagaaaataagggacatagagaacaaaataaaatatgagcagtagcaagaaattggccggtcttagggacaatttcagtttgctcggcgaaaagaataagatcttggtcgccaatagaggtgaaattccgattagaatttttagatctgctcatgagctgtctatgagaaccatcgccatatactcccatgaggaccgtctttcaatgcacaggttgaaggcggacgaagcgtatgttatcggggaggagggccagtatacacctgtgggtgcttacttggcaatggacgagatcatcgaaattgcaaagaagcataaggtggatttcatccatccaggttatgggttcttgtctgaaaattcggaatttgccgacaaagtagtgaaggccggtatcacttggatcggccctccagctgaagttattgactctgtgggtgacaaagtctctgccagacacttggcagcaagagctaacgttcctaccgttcccggtactccaggacctatcgaaactgtgcaagaggcacttgacttcgttaatgaatacggctacccggtgatcattaaggccgcctttggtggtggtggtagaggtatgagagtcgttagagaaggtgacgacgtggcagatgcctttcaacgtgctacctccgaagcccgtactgccttcggtaatggtacctgctttgtggaaagattcttggacaagccaaagcatattgaagttcaattgttggctgataaccacggaaacgtggttcatcttttcgaaagagactgttctgtgcaaagaagacaccaaaaagttgtcgaagtcgctccagcaaagactttgccccgtgaagttcgtgacgctattttgacagatgctgttaaattagctaaggtatgtggttacagaaacgcaggtaccgccgaattcttggttgacaaccaaaacagacactatttcattgaaattaatccaagaattcaagtggagcataccatcactgaagaaatcaccggtattgacattgtttctgcccaaatccagattgccgcaggtgccactttgactcaactaggtctattacaggataaaatcaccacccgtgggttttccatccaatgtcgtattaccactgaagatccctctaagaatttccaaccggataccggtcgcctggaggtctatcgttctgccggtggtaatggtgtgagattggacggtggtaacgcttatgcaggtgctactatctcgcctcactacgactcaatgctggtcaaatgttcatgctctggttctacttatgaaatcgtccgtaggaagatgattcgtgccctgatcgaattcagaatcagaggtgttaagaccaacattcccttcctattgactcttttgaccaatccagtttttattgagggtacatactggacgacttttattgacgacaccccacaactgttccaaatggtatcgtcacaaaacagagcgcaaaaactgttacactatttggcagacttggcagttaacggttcttctattaagggtcaaattggcttgccaaaactaaaatcaaatccaagtgtcccccatttgcacgatgctcagggcaatgtcatcaacgttacaaagtctgcaccaccatccggatggagacaagtgctactggaaaagggaccatctgaatttgccaagcaagtcagacagttcaatggtactctactgatggacaccacctggagagacgctcatcaatctctacttgcaacaagagtcagaacccacgatttggctacaatcgctccaacaaccgcacatgcccttgcaggtgctttcgctttagaatgttggggtggtgctacattcgacgttgcaatgagattcttgcatgaggatccatgggaacgtctgagaaaattaagatctctggtgcctaatattccattccaaatgttattacgtggtgccaacggtgtggcttactcttcattacctgacaatgctattgaccattttgtcaagcaagccaaggataatggtgttgatatatttagagtttttgatgccttgaatgatttagaacaattaaaagttggtgtgaatgctgtcaagaaggccggtggtgttgtcgaagctactgtttgttactctggtgacatgcttcagccaggtaagaaatacaacttagactactacctagaagttgttgaaaaaatagttcaaatgggtacacatatcttgggtattaaggatatggcaggtactatgaaaccggccgctgccaaattattaattggctccctaagaaccagatatccggatttaccaattcatgttcacagtcatgactccgcaggtactgctgttgcgtctatgactgcatgtgccctagcaggtgctgatgttgtcgatgtagctatcaattcaatgtcgggcttaacttcccaaccatcaattaatgcactgttggcttcattagaaggtaacattgatactgggattaacgttgagcatgttcgtgaattagatgcatactgggccgaaatgagactgttgtattcttgtttcgaggccgacttgaagggaccagatccagaagtttaccaacatgaaatcccaggtggtcaattgactaacttgttattccaagctcaacaactgggtcttggtgaacaatgggctgaaactaaaagagcttacagagaagccaattacctactgggagatattgttaaagttaccccaacttctaaggttgtcggtgatttagctcaattcatggtttctaacaaactgacttccgacgatattagacgtttagctaattctttggactttcctgactctgttatggacttttttgaaggtttaattggtcaaccatacggtgggttcccagaaccattaagatctgatgtattgagaaacaagagaagaaagttgacgtgccgtccaggtttagaattagaaccatttgatctcgaaaaaattagagaagacttgcagaacagattcggtgatattgatgaatgcgatgttgcttcttacaatatgtatccaagggtctatgaagatttccaaaagatcagagaaacatacggtgatttatcagttctaccaaccaaaaatttcctagcaccagcagaacctgatgaagaaatcgaagtcaccatcgaacaaggtaagactttgattatcaaattgcaagctgttggtgacttaaataagaaaactgggcaaagagaagtgtattttgaattgaacggtgaattaagaaagatcagagttgcagacaagtcacaaaacatacaatctgttgctaaaccaaaggctgatgtccacgatactcaccaaatcggtgcaccaatggctggtgttatcatagaagttaaagtacataaagggtctttggtgaaaaagggcgaatcgattgctgttttgagtgccatgaaaatggaaatggttgtctcttcaccagcagatggtcaagttaaagacgttttcattaaggatggtgaaagtgttgacgcatcagatttgttggttgtcctagaagaagaaaccctacccccatcccaaaaaaagtaatttttactcgttaattatattttatgacatctgaaaatactagctgtactatatatggcgtatattttatctagttatgttcccatgtatatttaaatgccaaatagaaagtaatcaaacactttcgatgaaatacgtgctaactgtgtttcttccttaatgctttcacttaccatgtctccattctccattttcttcttgagtgaaaatgtgagtttataacgctcaagtacgttaactactctatttaatatcgtacgggatttttgatcgactgtaggttttcttcttagaccattccagcgc

SEQ ID NO: 5 - DNA, 1044 bp, Saccharomyces cerevisiae
gctctagaaacatggtcaaagtcgcaattcttggcgcttctggtggcgtgggacaaccgctatcattactgctaaaattaagcccttacgtttccgagctggcgttgtacgatatccgagctgcggaaggcattggtaaggatttatctcacatcaacaccaactcaagttgtgtcggttatgataaggatagtattgagaacaccttgtcaaatgctcaggtggtgctaataccggctggtgttcccagaaagcccggtttaactagagatgatttgttcaagatgaacgccggtattgtcaaaagcctggtaaccgctgttggaaagttcgcaccaaatgcgaggattttagtcatttcaaaccctgtaaacagtttggtccctattgctgtggaaactttgaagaaaatgggtaagttcaaacctggaaacgttatgggtgtgacgaaccttgacctggtacgtgcagaaacctttttggtagattatttgatgctaaaaaaccccaaaattggacaagaacaagacaaaactacaatgcacagaaaggtcactgttattgggggtcattcaggggaaaccattatcccaataatcaccgacaaatcgctggtatttcaacttgataagcagtacgagcacttcattcatagggtccagttcggaggtgatgaaattgtcaaagctaaacagggcgccggttccgccacgttgtccatggcgttcgcgggggccaagtttgctgaagaagttttgaggagcttccataatgagaaaccagaaacggagtcactttccgcattcgtttatttaccaggcttaaaaaacggtaagaaagcgcagcaattagttggcgacaactctattgagtatttttccttgccaattgttttgagaaatggtagcgtagtatccatcgataccagtgttctggaaaaactgtctccgagagaggaacaactcgttaatactgcggtcaaagagctacgcaagaatattgaaaaaggcaagagtttcatcctagactcttgagtcgacagct

SEQ ID NO: 6 - DNA, 1336 bp, Schizosaccharomyces pombe
gctctagacatgggtgaactcaaggaaatcttgaaacagaggtatcatgagttgcttgactggaatgtcaaagcccctcatgtccctctcagtcaacgactgaagcattttacatggtcttggtttgcatgtactatggcaactggtggtgttggtttgattattggttctttcccctttcgattttatggtcttaatacaattggcaaaattgtttatattcttcaaatctttttgttttctctctttggatcatgcatgctttttcgctttattaaatatccttcaactatcaaggattcctggaaccatcatttggaaaagcttttcattgctacttgtcttctttcaatatccacgttcatcgacatgcttgccatatacgcctatcctgataccggcgagtggatggtgtgggtcattcgaatcctttattacatttacgttgcagtatcctttatatactgcgtaatggctttttttacaattttcaacaaccatgtatataccattgaaaccgcatctcctgcttggattcttcctattttccctcctatgatttgtggtgtcattgctggcgccgtcaattctacacaacccgctcatcaattaaaaaatatggttatctttggtatcctctttcaaggacttggtttttgggtttatcttttactgtttgccgtcaatgtcttacggttttttactgtaggcctggcaaaaccccaagatcgacctggtatgtttatgtttgtcggtccaccagctttctcaggtttggccttaattaatattgcgcgtggtgctatgggcagtcgcccttatatttttgttggcgccaactcatccgagtatcttggttttgtttctacctttatggctatttttatttggggtcttgctgcttggtgttactgtctcgccatggttagctttttagcgggctttttcactcgagcccctctcaagtttgcttgtggatggtttgcattcattttccccaacgtgggttttgttaattgtaccattgagataggtaaaatgatagattccaaagctttccaaatgtttggacatatcattggggtcattctttgtattcagtggatcctcctaatgtatttaatggtccgtgcgtttctcgtcaatgatctttgctatcctggcaaagacgaagatgcccatcctccaccaaaaccaaatacaggtgtccttaaccctaccttcccacctgaaaaagcacctgcatctttggaaaaagtcgatacacatgtcacatctactggtggtgaatcggatcctcctagtagtgaacatgaaagcgtttaagtcgacgcgt

SEQ ID NO: 7 - protein, 877 aa, Escherichia coli
MNEQYSALRSNVSMLGKVLGETIKDALGEHIRVETIRKLSKSSRAGNDANRQELLTTLQNLSNDELLPVARAFSQFLNLANTAEQYHSISPKGEANPEVIARTLRKLKNQPELSEDTIKKAVESLSLELVLTAHPTEITRRTLIHKMVEVNACLKQLDNKDIADYEHNQLMRRLRQLIAQSWHTDEIRKLRPSPVDEAKWGFAVVENSLWQGVPNYLRELNEQLEENLGYKLPVEFVPVRFTSWMGGDRDGNPNVTADITRHVLLLSRWKATDLFLKDIQVLVSELSMVEATPELLALVGEEGAAEPYRYLMKNLRSRLMATQAWLEARLKGEELPKPEGLLTQNEELWEPLYACYQSLQACGMGIIANGDLLDTLRRVKCFGVPLVRIDIRQESTRHTEALGELTRYLGIGDYESWSEADKQAFLIRELNSKRPLLPRNWQPSAETREVLDTCQVIAEAPQGSIAAYVISMAKTPSDVLAVHLLLKEAGIGFAMPVAPLFETLDDLNNANDVMTQLLNIDWYRGLIQGKQMVMIGYSDSAKDAGVMAASWAQYQAQDALIKTCEKAGIELTLFHGRGGSIGRGGAPAHAALLSQPPGSLKGGLRVTEQGEMIRFKYGLPEITVSSLSLYTGAIANLLPPPEPKESWRRIMDELSVISCDVYRGYVRENKDFVPYFRSATPEQELGKLPLGSRPAKRRPTGGVESLRAIPWIFAWTQNRLMLPAWLGAGTALQKVVEDGKQSELEAMCRDWPFFSTRLGMLEMVFAKADLWLAEYYDQRLVDKALWPLGKELRNLQEEDIKVVLAIANDSHLMADLPWIAESIQLRNIYTDPLNVLQAELLHRSRQAEKEGQEPDPRVEQALMVTIAGIAAGMRNTG

SEQ ID NO: 8 - DNA, 2652 bp, Escherichia coli
atgaacgaacaatattccgcattgcgtagtaatgtcagtatgctcggcaaagtgctgggagaaaccatcaaggatgcgttgggagaacacattcttgaacgcgtagaaactatccgtaagttgtcgaaatcttcacgcgctggcaatgatgctaaccgccaggagttgctcaccaccttacaaaatttgtcgaacgacgagctgctgcccgttgcgcgtgcgtttagtcagttcctgaacctggccaacaccgccgagcaataccacagcatttcgccgaaaggcgaagctgccagcaacccggaagtgatcgcccgcaccctgcgtaaactgaaaaaccagccggaactgagcgaagacaccatcaaaaaagcagtggaatcgctgtcgctggaactggtcctcacggctcacccaaccgaaattacccgtcgtacactgatccacaaaatggtggaagtgaacgcctgtttaaaacagctcgataacaaagatatcgctgactacgaacacaaccagctgatgcgtcgcctgcgccagttgatcgcccagtcatggcataccgatgaaatccgtaagctgcgtccaagcccggtagatgaagccaaatggggctttgccgtagtggaaaacagcctgtggcaaggcgtaccaaattacctgcgcgaactgaacgaacaactggaagagaacctcggctacaaactgcccgtcgaatttgttccggtccgttttacttcgtggatgggcggcgaccgcgacggcaacccgaacgtcactgccgatatcacccgccacgtcctgctactcagccgctggaaagccaccgatttgttcctgaaagatattcaggtgctggtttctgaactgtcgatggttgaagcgacccctgaactgctggcgctggttggcgaagaaggtgccgcagaaccgtatcgctatctgatgaaaaacctgcgttctcgcctgatggcgacacaggcatggctggaagcgcgcctgaaaggcgaagaactgccaaaaccagaaggcctgctgacacaaaacgaagaactgtgggaaccgctctacgcttgctaccagtcacttcaggcgtgtggcatgggtattatcgccaacggcgatctgctcgacaccctgcgccgcgtgaaatgtttcggcgtaccgctggtccgtattgatatccgtcaggagagcacgcgtcataccgaagcgctgggcgagctgacccgctacctcggtatcggcgactacgaaagctggtcagaggccgacaaacaggcgttcctgatccgcgaactgaactccaaacgtccgcttctgccgcgcaactggcaaccaagcgccgaaacgcgcgaagtgctcgatacctgccaggtgattgccgaagcaccgcaaggctccattgccgcctacgtgatctcgatggcgaaaacgccgtccgacgtactggctgtccacctgctgctgaaagaagcgggtatcgggtttgcgatgccggttgctccgctgtttgaaaccctcgatgatctgaacaacgccaacgatgtcatgacccagctgctcaatattgactggtatcgtggcctgattcagggcaaacagatggtgatgattggctattccgactcagcaaaagatgcgggagtgatggcagcttcctgggcgcaatatcaggcacaggatgcattaatcaaaacctgcgaaaaagcgggtattgagctgacgttgttccacggtcgcggcggttccattggtcgcggcggcgcacctgctcatgcggcgctgctgtcacaaccgccaggaagcctgaaaggcggcctgcgcgtaaccgaacagggcgagatgatccgctttaaatatggtctgccagaaatcaccgtcagcagcctgtcgctttataccggggcgattctggaagccaacctgctgccaccgccggagccgaaagagagctggcgtcgcattatggatgaactgtcagtcatctcctgcgatgtctaccgcggctacgtacgtgaaaacaaagattttgtgccttacttccgctccgctacgccggaacaagaactgggcaaactgccgttgggttcacgtccggcgaaacgtcgcccaaccggcggcgtcgagtcactacgcgccattccgtggatcttcgcctggacgcaaaaccgtctgatgctccccgcctggctgggtgcaggtacggcgctgcaaaaagtggtcgaagacggcaaacagagcgagctggaggctatgtgccgcgattggccattcttctcgacgcgtctcggcatgctggagatggtcttcgccaaagcagacctgtggctggcggaatactatgaccaacgcctggtagacaaagcactgtggccgttaggtaaagagttacgcaacctgcaagaagaagacatcaaagtggtgctggcgattgccaacgattcccatctgatggccgatctgccgtggattgcagagtctattcagctacggaatatttacaccgacccgctgaacgtattgcaggccgagttgctgcaccgctcccgccaggcagaaaaagaaggccaggaaccggatcctcgcgtcgaacaagcgttaatggtcactattgccgggattgcggcaggtatgcgtaataccggctaa

SEQ ID NO: 9 - protein, 334 aa, Saccharomyces cerevisiae
MLSRVAKRAFSSTVANPYKVTVLGAGGGIGQPLSLLLKLNHKVTDLRLYDLKGAKGVATDLSHIPTNSVVKGFTPEEPDGLNNALKDTDMVLIPAGVPRKPGMTRDDLFAINASIVRDLAAATAESAPNAAILVISNPVNSTVPIVAQVLKNKGVYNPKKLFGVTTLDSIRAARFISEVENTDPTQERVNVIGGHSGITIIPLISQTNHKLMSDDKRHELIHRIQFGGDEVVKAKNGAGSATLSMAHAGAKFANAVLSGFKGERDVIEPSFVDSPLFKSEGIEFFASPVTLGPDGIEKIHPIGELSSEEEEMLQKCKETLKKNIEKGVNFVASK

SEQ ID NO: 10 - DNA, 1844 bp, Saccharomyces cerevisiae
tgattacgctaataacactacataagaaacatagataaattataattcactaatacaaaatccctcaatttaattaatcaatttcttattcaacaactagagtaggcactagcagtttgtgcgaccctcggcgccggagaggtacaagggacctggcggttgggccttcctgccagcttctcccccccttctcggcattcactcagcgttcattggctcaatcacgcgccaagcggattcccagaaaaatacaaaaaaaagttgccataatacaatggcggatattacaatatatatagcttgcgctttaaaaagaagtgttagcacgcattgggtgtttgtactgtattcctccacttaagataacgaatacacatatagatatacaatatattatacgtggacatctacggaaaggaagaaaaaaaacaaaaggaaaaggaaggataccatatacaatgttgtcaagagtagctaaacgtgcgttttcctctacagttgccaacccttataaagtgactgttttgggtgcaggcggtggtattggacaaccattgtctttgcttctaaagcttaaccataaagtcacggacttaagactgtacgacctaaagggcgcaaaaggtgttgccaccgatttgtctcatattccaacaaactccgtggtcaaggggtttactccagaagagccagacggattgaacaacgctttaaaggacacagacatggttttaattcctgctggtgtgcccagaaagcctggtatgacacgtgatgacttgttcgccatcaacgcaagcatcgttcgcgatttggcagcagcaaccgccgaatccgctcccaatgctgccattctggtcatttccaacccagtcaattctaccgttccaattgtcgcccaagtcttgaaaaacaagggtgtttacaacccaaagaaattgttcggtgtgactaccttggactctattagagccgccagattcatctcagaagtcgagaacaccgatccaactcaggaaagggttaacgtcatcggtggacattctggtattaccatcatcccattgatttcgcaaacaaaccataagttgatgtctgatgacaagagacacgaattgattcacagaatacagtttggtggtgacgaagtcgtcaaagcaaagaatggtgctggctctgctacgttgtcaatggcccatgctggtgctaaattcgctaacgctgttttgtccggtttcaaaggcgaaagagacgtcatcgagccttccttcgtggactctcccttgttcaaatccgaaggcatcgaattctttgcatctccggtcactttgggcccagatggtattgaaaagatccatccaataggtgagttatcttcagaagaagaagaaatgctacaaaaatgtaaagaaaccttgaagaagaatatcgaaaagggtgtcaactttgttgctagtaaatagattgttcaagatcagaaatagagtgaaaaatagggaaaaaaaaaaaaaaattctactaataagaacggaagactactcgccatcatgagattacgacatcttttttatttaattctttgttatatttagccaattgaaaggaaggaaactgccaaaaatcaactagagaaaaattcatgctattcgttgctcctacacctttttttcttccgtagacgaggaaaaacaaaaatgattaaataggcgcgcgcggtgtgatggtgatgataatgatgatgatgatgataatgatgaataaacgtttcggtttacgagtttctttaaatatacatatatgatataaaaaaaaaaatacgtaccacttacaatttgcaaatatag

SEQ ID NO: 11 - protein, 377 aa, Saccharomyces cerevisiae
MPHSVTPSIEQDSLKIAILGAAGGIGQSLSLLLKAQLQYQLKESNRSVTHIHLALYDVNQEAINGVTADLSHIDTPISVSSHSPAGGIENCLHNASIVVIPAGVPRKPGMTRDDLFNVNAGIISQLGDSIAECCDLSKVFVLVISNPVNSLVPVMVSNILKNHPQSRNSGIERRIMGVTKLDIVRASTFLREINIESGLTPRVNSMPDVPVIGGHSGETIIPLFSQSNFLSRLNEDQLKYLIHRVQYGGDEVVKAKNGKGSATLSMAHAGYKCVVQFVSLLLGNIEQIHGTYYVPLKDANNFPIAPGADQLLPLVDGADYFAIPLTITTKGVSYVDYDIVNRMNDMERNQMLPICVSQLKKNIDKGLEFVASRSASS

SEQ ID NO: 12 - DNA, 1134 bp, Saccharomyces cerevisiae
atgcctcactcagttacaccatccatagaacaagattcgttaaaaattgccattttaggtgctgccggtggtatcgggcagtcgttatcgctgcttttgaaagctcagttgcaataccagttaaaggagagcaaccggagcgttacccacattcatctggctctttacgatgtcaaccaagaagccatcaacggtgttaccgccgacttgtctcatatagacacccccatttccgtgtcgagccactctcctgcaggtggcattgagaactgtttgcataacgcttctattgttgtcattcctgcaggtgttccaagaaaacctggcatgactcgtgatgacttatttaacgtgaatgctggtatcattagccagctcggtgattctattgcagaatgttgtgatctttccaaggtcttcgttcttgtcatttccaaccctgttaattctttagtcccagtgatggtttctaacattcttaagaaccatcctcagtctagaaattccggcattgaaagaaggatcatgggtgtcaccaagctcgacattgtcagagcgtccacttttctacgtgagataaacattgagtcagggctaactcctcgtgttaactccatgcctgacgtccctgtaattggcgggcattctggcgagactattattccgttgttttcacagtcaaacttcctatcgagattaaatgaggatcaattgaaatatttaatacatagagtccaatacggtggtgatgaagtggtcaaggccaagaacggtaaaggtagtgctaccttatcgatggcccatgccggttataagtgtgttgtccaatttgtttctttgttattgggtaacattgagcagatccatggaacctactatgtgccattaaaagatgcgaacaacttccccattgctcctggggcagatcaattattgcctctggtggacggtgcagactactttgccataccattaactattactacaaagggtgtttcctatgtggattatgacatcgttaataggatgaacgacatggaacgcaaccaaatgttgccaatttgcgtctcccagttaaagaaaaatatcgataagggcttggaattcgttgcatcgagatctgcatcatcttaa

SEQ ID NO: 13 - protein, 377 aa, Saccharomyces cerevisiae
MSHSVTPSIEQDSLKIAILGAAGGIGQSLSLLLKAQLQYQLKESNRSVTHIHLALYDVNQEAINGVTADLSHIDTPISVSSHSPAGGIENCLHNASIVVIPAGVPRKPGMTRDDLFNVNAGIISQLGDSIAECCDLSKVFVLVISNPVNSLVPVMVSNILKNHPQSRNSGIERRIMGVTKLDIVRASTFLREINIESGLTPRVNSMPDVPVIGGHSGETIIPLFSQSNFLSRLNEDQLKYLIHRVQYGGDEVVKAKNGKGSATLSMAHAGYKCVVQFVSLLLGNIEQIHGTYYVPLKDANNFPIAPGADQLLPLVDGADYFAIPLTITTKGVSYVDYDIVNRMNDMERNQMLPICVSQLKKNIDKGLEFVASRSASS

SEQ ID NO: 14 - DNA, 1134 bp, Saccharomyces cerevisiae
atgtctcactcagttacaccatccatagaacaagattcgttaaaaattgccattttaggtgctgccggtggtatcgggcagtcgttatcgctgcttttgaaagctcagttgcaataccagttaaaggagagcaaccggagcgttacccacattcatctggctctttacgatgtcaaccaagaagccatcaacggtgttaccgccgacttgtctcatatagacacccccatttccgtgtcgagccactctcctgcaggtggcattgagaactgtttgcataacgcttctattgttgtcattcctgcaggtgttccaagaaaacctggcatgactcgtgatgacttatttaacgtgaatgctggtatcattagccagctcggtgattctattgcagaatgttgtgatctttccaaggtcttcgttcttgtcatttccaaccctgttaattctttagtcccagtgatggtttctaacattcttaagaaccatcctcagtctagaaattccggcattgaaagaaggatcatgggtgtcaccaagctcgacattgtcagagcgtccacttttctacgtgagataaacattgagtcagggctaactcctcgtgttaactccatgcctgacgtccctgtaattggcgggcattctggcgagactattattccgttgttttcacagtcaaacttcctatcgagattaaatgaggatcaattgaaatatttaatacatagagtccaatacggtggtgatgaagtggtcaaggccaagaacggtaaaggtagtgctaccttatcgatggcccatgccggttataagtgtgttgtccaatttgtttctttgttattgggtaacattgagcagatccatggaacctactatgtgccattaaaagatgcgaacaacttccccattgctcctggggcagatcaattattgcctctggtggacggtgcagactactttgccataccattaactattactacaaagggtgtttcctatgtggattatgacatcgttaataggatgaacgacatggaacgcaaccaaatgttgccaatttgcgtctcccagttaaagaaaaatatcgataagggcttggaattcgttgcatcgagatctgcatcatcttaa

SEQ ID NO: 15 - protein, 343 aa, Saccharomyces cerevisiae
MVKVAILGASGGVGQPLSLLLKLSPYVSELALYDIRAAEGIGKDLSHINTNSSCVGYDKDSIENTLSNAQVVLIPAGVPRKPGLTRDDLFKMNAGIVKSLVTAVGKFAPNARILVISNPVNSLVPIAVETLKKMGKFKPGNVMGVTNLDLVRAETFLVDYLMLKNPKIGQEQDKTTMHRKVTVIGGHSGETIIPIITDKSLVFQLDKQYEHFIHRVQFGGDEIVKAKQGAGSATLSMAFAGAKFAEEVLRSFHNEKPETESLSAFVYLPGLKNGKKAQQLVGDNSIEYFSLPIVLRNGSVVSIDTSVLEKLSPREEQLVNTAVKELRKNIEKGKSFILDSSKL

SEQ ID NO: 16 - DNA, 1032 bp, Saccharomyces cerevisiae
atggtcaaagtcgcaattcttggcgcttctggtggcgtgggacaaccgctatcattactgctaaaattaagcccttacgtttccgagctggcgttgtacgatatccgagctgcggaaggcattggtaaggatttatctcacatcaacaccaactcaagttgtgtcggttatgataaggatagtattgagaacaccttgtcaaatgctcaggtggtgctaataccggctggtgttcccagaaagcccggtttaactagagatgatttgttcaagatgaacgccggtattgtcaaaagcctggtaaccgctgttggaaagttcgcaccaaatgcgaggattttagtcatttcaaaccctgtaaacagtttggtccctattgctgtggaaactttgaagaaaatgggtaagttcaaacctggaaacgttatgggtgtgacgaaccttgacctggtacgtgcagaaacctttttggtagattatttgatgctaaaaaaccccaaaattggacaagaacaagacaaaactacaatgcacagaaaggtcactgttattgggggtcattcaggggaaaccattatcccaataatcaccgacaaatcgctggtatttcaacttgataagcagtacgagcacttcattcatagggtccagttcggaggtgatgaaattgtcaaagctaaacagggcgccggttccgccacgttgtccatggcgttcgcgggggccaagtttgctgaagaagttttgaggagcttccataatgagaaaccagaaacggagtcactttccgcattcgtttatttaccaggcttaaaaaacggtaagaaagcgcagcaattagttggcgacaactctattgagtatttttccttgccaattgttttgagaaatggtagcgtagtatccatcgataccagtgttctggaaaaactgtctccgagagaggaacaactcgttaatactgcggtcaaagagctacgcaagaatattgaaaaaggcaagagtttcatcctagactcttccaagctatga

SEQ ID NO: 17 - protein, 340 aa, Saccharomyces cerevisiae; identical to SEQ ID NO: 2

SEQ ID NO: 18 - DNA, 1023 bp, Saccharomyces cerevisiae
atggtcaaagtcgcaattcttggcgcttctggtggcgtgggacaaccgctatcattactgctaaaattaagcccttacgtttccgagctggcgttgtacgatatccgagctgcggaaggcattggtaaggatttatctcacatcaacaccaactcaagttgtgtcggttatgataaggatagtattgagaacaccttgtcaaatgctcaggtggtgctaataccggctggtgttcccagaaagcccggtttaactagagatgatttgttcaagatgaacgccggtattgtcaaaagcctggtaaccgctgttggaaagttcgcaccaaatgcgaggattttagtcatttcaaaccctgtaaacagtttggtccctattgctgtggaaactttgaagaaaatgggtaagttcaaacctggaaacgttatgggtgtgacgaaccttgacctggtacgtgcagaaacctttttggtagattatttgatgctaaaaaaccccaaaattggacaagaacaagacaaaactacaatgcacagaaaggtcactgttattgggggtcattcaggggaaaccattatcccaataatcaccgacaaatcgctggtatttcaacttgataagcagtacgagcacttcattcatagggtccagttcggaggtgatgaaattgtcaaagctaaacagggcgccggttccgccacgttgtccatggcgttcgcgggggccaagtttgctgaagaagttttgaggagcttccataatgagaaaccagaaacggagtcactttccgcattcgtttatttaccaggcttaaaaaacggtaagaaagcgcagcaattagttggcgacaactctattgagtatttttccttgccaattgttttgagaaatggtagcgtagtatccatcgataccagtgttctggaaaaactgtctccgagagaggaacaactcgttaatactgcggtcaaagagctacgcaagaatattgaaaaaggcaagagtttcatcctagactcttga

SEQ ID NO: 19 - protein, 312 aa, Actinobacillus succinogenes
MKVTLLGASGGIGQPLSLLLKLHLPAESDLSLYDVAPVTPGVAKDISHIPTSVEVEGFGGDDPSEALKGADIVLICAGVARKPGMTRADLFNVNAGIIQNLVEKVAQVCPQACVCIITNPVNSIIPIAAEVLKKAGVYDKRKLFGITTLDTIRSEKFIVQAKNIEINRNDISVIGGHSGVTILPLLSQIPHVEFTEQELKDLTHRIQNAGTEVVEAKAGAGSATLSMAYAAMRFVVSMARALNGEVITECAYIEGDGKFARFFAQPVRLGKNGVEEILPLGTLSAFEQQALEAMLPTLQTDIDNGVKFVTGE

SEQ ID NO: 20 - DNA, 1242 bp, Actinobacillus succinogenes
atgaaagtaaccttattaggcgccagcggcggtatcggtcaacctctttcattgttgttaaaattacatcttccggcagaaagcgatttaagcttatacgatgttgcgccggtcacccccggtgtggcgaaagacatcagccatattccgacttcggttgaagtggaaggtttcggcggcgatgatccgtccgaggcattaaaaggggcggatatcgttttaatctgtgcgggtgtggcgcgtaagccgggtatgactcgtgcggatttgtttaatgttaacgccggtattatccagaatttagtggaaaaagttgcgcaagtttgcccgcaggcttgtgtttgcattatcactaatccggtgaactcgattattccgattgcggcggaagtgctgaaaaaagcgggcgtatacgataaacggaaattattcggtattactacgctggataccatccgttccgaaaaatttatcgtgcaagcgaaaaatattgaaatcaaccgtaacgatatttcagttatcggcggacattcaggtgtgacgattttacctttgttgtcacaaattccgcatgtggaatttaccgagcaggaattaaaagatttaactcaccgcatccaaaatgccggcaccgaagtggtagaagctaaagccggtgcgggttccgctacactttccatggcgtatgcggcaatgcgttttgtggtttccatggctcgcgcattaaacggcgaagtgattacggaatgcgcctatattgaaggcgacggtaaattcgcccgtttctttgcacaaccggttcgtttgggtaaaaacggcgtagaagaaattctgccgttaggtacattaagcgcatttgagcaacaagcgcttgaagcgatgttaccgaccttgcaaactgacattgataacggtgtgaaatttgttaccggcgaataattcaccaaaataatttaacaaaaccgattaaaggattaggtttttatgcaaacctaatcctttttgtttggtatcaatcagttaaaatccgccgtttgattaatgggaagctatataagattttagtattttatatagataaaaatagcgtggaagaataaagtaatcctccacgcgtcttctcaaatgtataaaaagtgcggtcaaaaattaatcgattttttattcatcctcgtttcttggcggtttaatcgccagtaaattacattttaacttactgataacatgttcggcggtgttgct

SEQ ID NO: 21 - protein, 331 aa, Yarrowia lipolytica
VVKAVVAGAAGGIGQPLSLLLKLSPYVTELALYDVVNSPGVAADLSHISTKAKVTGYLPKDDGLKNALTGANIVVIPAGIPRKPGMTRDDLFKINAGIVRDLVTGVAQYAPDAFVLIISNPVNSTVPIAAEVLKKHNVFNPKKLFGVTTLDVVRAQTFTAAVVGESDPTKLNIPVVGGHSGDTIVPLLSLTKPKVEIPADKLDDLVKRIQFGGDEVVQAKDGLGSATLSMAQAGFRFAEAVLKGAAGEKGIIEPAYIYLDGIDGTSDIKREVGVAFFSVPVEFGPEGAAKAYNILPEANDYEKKLLKVSIDGLYGNIAKGEEFIVNPPPAK

SEQ ID NO: 22 - DNA, 996 bp, Yarrowia lipolytica
gtggttaaagctgtcgttgccggagccgctggtggtattggccagcccctttctcttctcctcaaactctctccttacgtgaccgagcttgctctctacgatgtcgtcaactcccccggtgttgccgctgacctctcccacatctccaccaaggctaaggtcactggctacctccccaaggatgacggtctcaagaacgctctgaccggcgccaacattgtcgttatccccgccggtatcccccgaaagcccggtatgacccgagacgatctgttcaagatcaacgctggtatcgtccgagatctcgtcaccggtgtcgcccagtacgcccctgacgcctttgtgctcatcatctccaaccccgtcaactctaccgtccctattgctgccgaggtcctcaagaagcacaacgtcttcaaccctaagaagctcttcggtgtcaccacccttgacgttgtccgagcccagaccttcaccgccgctgttgttggcgagtctgaccccaccaagctcaacatccccgtcgttggtggccactccggagacaccattgtccctctcctgtctctgaccaagcctaaggtcgagatccccgccgacaagctcgacgacctcgtcaagcgaatccagtttggtggtgacgaggttgtccaggctaaggacggtcttggatccgctaccctctccatggcccaggctggtttccgatttgccgaggctgtcctcaagggtgccgctggtgagaagggcatcatcgagcccgcctacatctaccttgacggtattgatggcacctccgacatcaagcgagaggtcggtgtcgccttcttctctgtccctgtcgagttcggccctgagggtgccgctaaggcttacaacatccttcccgaggccaacgactacgagaagaagcttctcaaggtctccatcgacggtctttacggcaacattgccaagggcgaggagttcattgttaaccctcctcctgccaagtaa

SEQ ID NO: 23 - protein, 330 aa, Aspergillus niger
MVKAAVLGASGGIGQPLSLLLKTSPLVDDLALYDVVNTPGVAADLSHISSVAKISGFLPKDDGLKHALTGADIVVIPAGIPRKPGMTRDDLFKINAGIVRDLVKGIAEYCPKAFVLIISNPVNSTVPIAAEVLKAAGVFDPKRLFGVTTLDVVRAETFTQEFSGHKDPSAVRIPVVGGHSGETIVPLFSKAAPAFQIPADKYDALVNRVQFGGDEVVKAKDGAGSATLSMAYAGFRFAQSVIKAAQGQSGIVEPTFVYLPGIAGGEDISKATGLEFFSTLVELGTNGAEKAINVLDGVTEKEKTLIEACTKGLKGNIEKGIEFVKNPPPK

SEQ ID NO: 24 - DNA, 1482 bp, Aspergillus niger
atggttaaagcgggtgagtggctctttcacggcgcctgcatttcgaccccgctttctagaacgagagcaaccccagactaatcactaatacatctcgacagctgttcttggagcatctggtggcattggccaggtacgtgtccctcatttgtccctatcagaactgcagcccgcgatgcagacctaccacattcttcaacttttactgactccccacgcgtttcccatcggttagcctttgtctctcctgctgaagacatcccccctggttgacgacctcgcgctctacgatgttgtgaacacccccggtgttgctgccgacctttctcacatctcctctgttgctgtatgtgtgaccgtaccacaacgcgctcccctatgccaaacaatttgacaccctttctgacattttcggcagaaaatctccggattcctgcccaaggatgatggattgaagcacgccttgactggcgctgacattgttgtcatccctgctggaatcccccgtgagtgtatcacctcccctgttcctagaggcgtctgactgacaaagccgctgctcataggcaagcctgggatgactcgtgatgatctcttcaaaatcaacgccggtattgtgcgcgacttggtcaagggtatcgctgagtactgccccaaggcatttgttttgatcatctccaaccctgtcaattcgactgttcccatcgccgcagaggtccttaaggctgctggcgtctttgaccccaagcgtctctttggtgttaccactctggatgttgtccgcgcggaaacctttactcaggaattttctggccacaaggacccatctgctgtgcgcatcccagttgtcggtggccactcgggagagactattgttcccctctttagcaaggccgctcccgctttccagatcccggcagacaagtacgatgcgctagtcaaccgtaagtggaccgtgcagtctcttggcaattgaatcgtttattgaaccatatgcaggcgttcagttcggtggagatgaggttgtcaaggccaaggatggtgccggctccgcgaccctgtctatggcctacgctggcttcaggtttgagtccttcatttacaaacatttggtgtcggggctaacttcatatcaagatttgctcagagcgttatcaaggctgcgcaaggccagtcgggtatcgtcgagcccacctttgtctatctgcctggtatcgctggcggcgaagatatttccaaggccactggcctcgagttcttctctaccctcgtagaacttggcgtaagtcattcattatgtgatatttgaatattgggcatcactctgactcgacaaatcagaccaacggggctgagaaggccatcaatgtacttgacggcgtgactgagaaggaaaagacgctcatcgaggcctgcaccaagggcttgaagggcaacatcgaaaagggaatcgaattcgtcaagaaccccccaccaaagtaa

SEQ ID NO: 25 - protein, 600 aa, Kluyveromyces lactis
MNNNNITPESDSMKSNDNDQTNDYMPDVADFDHTQTNTNEIARAISHPGSVLSRVASYVSRKDRYVDENGNEVWQDDEVSILMEEDETPDFTWKNIRHYAITRFTTLTELHRVSMENINPIPELRKMTLHNWNYFFMGYAAWLCAAWAFFAVSVSTAPLATLYGKETKDISWGLSLVLFVRSAGAIIFGIWTDNYSRKWPYITCLGLFLICQLCTPWAKTYTQFLGVRWISGIAMGGIYACASATAIEDAPVKARSFLSGLFFTAYAMGFIFAIIFYRAFLNVNGENYWKVQFWFSIWLPAVLILWRLVWPETKYFTKVLKARQLMRDDAIAKNGGQPLPKLSFKQKFANVKKTVSKYWLLFGYLILLLVGPNYLTHASQDLFPTMLRAQLRFSEDAVTVAIVVVCLGSIAGGMFFGQLMEITGRRVGLLLALIMAGCFTYPAFMLKTSSAVLGAGFMLWFSILGVWGVLPIHLSELSPPEARALVSGLAYQLGNLASAASVVIENDLADLYPIEWNSAVKVTNKDYSKVMAILTGSSVIFTFVLVFVGHEKFHRDLSSPHLKSYIERVDQTEEVAAMTGSTANSISSKPSDDQLEKVSV

SEQ ID NO: 26 - DNA, 2150 bp, Kluyveromyces lactis
tctccggtacctttttactatctctcactaaaataactctaattcagtatgagcccgctggatgtaccacacctgcgtttcttgatatataaaagtaactggtttggcgtctttttttcgacaatcaacgttcttccaatcttgttgtggttgtgtcttattattattgtaatgtatgtcttgtatgatctggagtaaccaaaaccaaaacagatttcaaccatgaataacaacaatattacaccagagtctgactctatgaaatctaatgataatgatcaaacaaacgattatatgccggatgtagcggatttcgaccatacgcaaaccaataccaatgagattgctagagccatttcccatcctggttcagttttgtcacgtgttgcatcgtatgtgtcaagaaaggacagatatgtcgatgaaaacggtaacgaggtgtggcaggacgatgaagtgtcgattcttatggaggaagacgaaaccccagatttcacttggaaaaacattcgtcactatgcgatcactagatttaccactttgaccgagcttcatagggtatcgatggaaaatatcaatccgatcccagagttgagaaagatgactttacacaactggaactatttctttatggggtatgcggcttggttatgtgccgcttgggcgttctttgcagtgtccgtttctacggctccattggctactttgtacggcaaagaaactaaggatatctcttggggtctttcgttggttcttttcgttagatctgctggtgccatcatcttcggtatctggaccgataactattcgagaaaatggccttacatcacttgtttgggtttattcttaatctgtcaactttgtaccccatgggccaagacttacactcagttcttgggtgttagatggatttctggtatcgctatgggtggtatctacgcttgtgcctctgccactgccattgaagatgccccagtgaaggcaagatccttcctatcaggtcttttctttaccgcatatgctatgggtttcatctttgccatcatcttttacagagctttcttgaacgttaacggtgaaaactactggaaggttcaattctggttctctatctggttaccagctgtgttgatcttatggagattggtttggcctgagactaaatatttcactaaggtgttaaaggctagacaattgatgagagacgatgccattgccaagaacggtggtcaaccattgccaaaattgtctttcaaacaaaaattcgctaacgttaagaaaactgtttctaaatactggttattgttcggttacttgattctcttgcttgttggtccaaactacttgacccatgcctctcaagatttgttcccaaccatgttacgtgctcaattgcgtttctctgaagacgctgttaccgttgccattgttgttgtatgtttgggttccatcgctggtggtatgttctttggtcaactaatggaaatcaccggtagaagagtaggtttgctcttggcccttatcatggccggttgtttcacttatccagctttcatgttgaagacaagttctgctgtgttgggtgcagggttcatgctttggttctccattcttggtgtctggggtgttcttccaatccatctttccgaattatctcctccagaagctagagctttggtctctggtcttgcctatcaattaggtaacttggcttctgccgcttcggtcgttatcgaaaacgatttggctgatttgtacccaatcgaatggaactccgcagtgaaagtaacaaacaaggattactctaaagttatggctatcttgactggttccagtgtcatcttcacttttgtcttggtatttgtcggtcacgagaagttccacagagatttatcctctcctcatttgaaatcttatatcgaaagagttgaccaaacggaagaagttgctgctatgacaggttccacggccaattctatttcttctaagccaagtgatgatcaattggaaaaggtttcagtttgattaatgattaacagaaactaaacctttatacaatwtcatgcccacacacgcaccatagcattataaaaaggaaaaaaggaaaacattaaattaaatgactacacccacactcatgtgcactttat

SEQ ID NO: 27 - protein, 543 aa, Kluyveromyces lactis
  MOD_RES (38)..(38): Any amino acid
  MOD_RES (197)..(197): Any amino acid
  MOD_RES (209)..(209): Any amino acid
  MOD_RES (235)..(235): Any amino acid
MAAESIVSRDESIASLEKAEGRITYLKPQSRITWSDAXKYLATRIPTLFPTKASIREARKEYPINPFPALRSMNWLQTQYFIVGFLAWTWDALDFFAVSLNMTNLAKDLDRPVKDISHAITLVLLLRVIGALIFGYLGDRYGRKYSFVLTMALIIVIQIGTGFVNSFSAFLGCRAIFGIIMGSVFGSAFLGCRAIFXIIMGSVFGVASXTALENAPNKAKSILSGIFQEGYAFGXLLGVVFQRAIVDNSPHGWRAIFWFSAGPPVLFIAWRLMLPESQHYVERVRLEKLENDGKSQFWKNAKLACSQYWLSMIYLVLLMAGFNFSSHGSQDLFPTMLTSQYQFSADASTVTNSVANLGAIAGGIIVAHASSFFGRRFSIIVCCIGGGAMLYPWGFVANKSGINASVFFLQFFVQGAWGIVPIHLTELAPTEFRALITGVAYQLGNMISSASSTIEASIGERFPLEGREDAYDYGKVMCIFMGCVFAYLLIVTVLGPENKGGELRLSTTGTEQDDEESQNNNIIRRNCRGWTSFGSKFQARNST

SEQ ID NO: 28 - DNA, 1632 bp, Kluyveromyces lactis
atggctgcagaatcaatagtgtctcgcgatgaatccatcgcttcacttgaaaaagcagaaggtagaatcacatatttgaaaccgcaatctaggatcacatggagtgatgctaasaaatatttggctacaagaatacctactttgttcccaacaaaagcatcgattagagaagcaaggaaagaataccctataaatcctttccctgccttacgttcgatgaactggttgcaaacacaatactttatcgttgggttcttagcatggacttgggatgcgttagatttctttgccgtttcattgaacatgacaaatttggccaaggatctagacagacctgtaaaagatatttctcatgccattactttggtgttgctattaagggtcatcggtgctcttatctttggttatttgggtgacagatatggtagaaaatactcatttgttttaactatggctctcattatcgttattcaaatcggtacagggttcgttaattctttctctgctttcttggggtgtagagctatctttggtatcattatgggatctgtatttggttctgctttcttggggtgtagagctatctttgktatcattatgggatctgtatttggtgttgcttctkccactgccttggaaaatgctccaaacaaggctaagtccatcctttctggtatattccaagaaggttatgctttcggtwatttattaggtgtcgtgttccaaagagctattgttgataattctccacatggttggagagctatattctggttcagtgccgggcccccagtgcttttcattgcttggaggttgatgttacctgaatcccaacactatgtcgaaagagtccgtttggaaaaattagaaaacgatgggaagtctcaattctggaagaatgctaagcttgcctgttctcaatattggctaagtatgatttacttggttcttttaatggcaggtttcaacttctcctcccatggttctcaagatcttttcccaacaatgttgacttctcaataccaattctccgctgatgcatcaactgtwacaaactctgttgcaaaccttggtgccatcgctggtggtatcattgttgcccatgcctcctctttctttggtcgtagattctctatcattgtatgttgtattggcggtggtgctatgttatacccatggggttttgttgctaataaatctggaattaatgcttcagtcttcttcttacaattcttcgtccaaggtgcttggggtattgtcccaattcatttgacggaattagccccaacggagttcagagctttgatcactggtgttgcttaccaattgggtaatatgatatctagtgcctcctcaactatcgaagcctccattggtgaaagattcccacttgaaggtagagaggacgcttatgattatggtaaggtgatgtgtatcttcatgggatgcgtgttcgcttacttgttgatcgtaaccgttttgggcccagagaacaagggcggtgagttgagattatccactacgggtacagaacaagacgatgaagaatctcaaaataacaatatcattcgaagaaattgtcgcggctggaccagtttcggatctaaatttcaagcaagaaattcaacataa

SEQ ID NO: 29 - protein, 616 aa, Saccharomyces cerevisiae
MSSSITDEKISGEQQQPAGRKLYYNTSTFAEPPLVDGEGNPINYEPEVYNPDHEKLYHNPSLPAQSIQDTRDDELLERVYSQDQGVEYEEDEEDKPNLSAASIKSYALTRFTSLLHIHEFSWENVNPIPELRKMTWQNWNYFFMGYFAWLSAAWAFFCVSVSVAPLAELYDRPTKDITWGLGLVLFVRSAGAVIFGLWTDKSSRKWPYITCLFLFVIAQLCTPWCDTYEKFLGVRWITGIAMGGIYGCASATAIEDAPVKARSFLSGLFFSAYAMGFIFAIIFYRAFGYFRDDGWKILFWFSIFLPILLIFWRLLWPETKYFTKVLKARKLILSDAVKANGGEPLPKANFKQKMVSMKRTVQKYWLLFAYLVVLLVGPNYLTHASQDLLPTMLRAQLGLSKDAVTVIVVVTNIGAICGGMIFGQFMEVTGRRLGLLIACTMGGCFTYPAFMLRSEKAILGAGFMLYFCVFGVWGILPIHLAELAPADARALVAGLSYQLGNLASAAASTIETQLADRYPLERDASGAVIKEDYAKVMAILTGSVFIFTFACVFVGHEKFHRDLSSPVMKKYINQVEEYEADGLSISDIVEQKTECASVKMIDSNVSKTYEEHIETV

SEQ ID NO: 30 - DNA, 1851 bp, Saccharomyces cerevisiae
atgtcgtcgtcaattacagatgagaaaatatctggtgaacagcaacaacctgctggcagaaaactatactataacacaagtacatttgcagagcctcctctagtggacggagaaggtaaccctataaattatgagccggaagtttacaacccggatcacgaaaagctataccataacccatcactgcctgcacaatcaattcaggatacaagagatgatgaattgctggaaagagtttatagccaggatcaaggtgtagagtatgaggaagatgaagaggataagccaaacctaagcgctgcgtccattaaaagttatgctttaacgagatttacgtccttactgcacatccacgagttttcttgggagaatgtcaatcccatacccgaactgcgcaaaatgacatggcagaattggaactatttttttatgggttattttgcgtggttgtctgcggcttgggccttcttttgcgtttcagtatcagtcgctccattggctgaactatatgacagaccaaccaaggacatcacctgggggttgggattggtgttatttgttcgttcagcaggtgctgtcatatttggtttatggacagataagtcttccagaaagtggccgtacattacatgtttgttcttatttgtcattgcacaactctgtactccatggtgtgacacatacgagaaatttctgggcgtaaggtggataaccggtattgctatgggaggaatttacggatgtgcttctgcaacagcgattgaagatgcacctgtgaaagcacgttcgttcctatcaggtctatttttttctgcttacgctatggggttcatatttgctatcattttttacagagcctttggctactttagggatgatggctggaaaatattgttttggtttagtatttttctaccaattctactaattttctggagattgttatggcctgaaacgaaatacttcaccaaggttttgaaagcccgtaaattaatattgagtgacgcagtgaaagctaatggtggcgagcctctaccaaaagccaactttaaacaaaagatggtatccatgaagagaacagttcaaaagtactggttgttgttcgcatatttggttgttttattggtgggtccaaattacttgactcatgcttctcaagacttgttgccaaccatgctgcgtgcccaattaggcctatccaaggatgctgtcactgtcattgtagtggttaccaacatcggtgctatttgtgggggtatgatatttggacagttcatggaagttactggaagaagattaggcctattgattgcatgcacaatgggtggttgcttcacctaccctgcatttatgttgagaagcgaaaaggctatattaggtgccggtttcatgttatatttttgtgtctttggtgtctggggtatcctgcccattcaccttgcagagttggcccctgctgatgcaagggctttggttgccggtttatcttaccagctaggtaatctagcttctgcagcggcttccacgattgagacacagttagctgatagatacccattagaaagagatgcctctggtgctgtgattaaagaagattatgccaaagttatggctatcttgactggttctgttttcatcttcacatttgcttgtgtttttgttggccatgagaaattccatcgtgatttgtcctctcctgttatgaagaaatatataaaccaagtggaagaatacgaagccgatggtctttcgattagtgacattgttgaacaaaagacggaatgtgcttcagtgaagatgattgattcgaacgtctcaaagacatatgaggagcatattgagaccgtttaa

SEQ ID NO: 31 - protein, 586 aa, Mus musculus
MATCWQALWAYRSYLIVLCLPIFLLPLPLIVQTKEAYCAYSIILMALLWCTEALPLAVTALFPIILFPLMGIMEASKVCLEYFKDTNILFVGGLMVAIAVEHWNLHKRIALGVLLIIGVRPALLLLGFMLVTAFLSMWISNTATTAMMLPIGYAVLEQLQGSQKDVEEGNSNPSFELQEASPQKEETKLDNGQAVSVSSEPRAQKTKEHHRFSQGLSLCICYSASIGGIATLTGTTPNLVLQGQVNSIFPENSNVVNFASWFGFAFPTMVILLLLAWLWLQVLFLGVNFRKNFGFGEGEEERKQAAFQVIKTQHRLLGPMSFAEKAVTFLFVLLVVLWFTREPGFFPGWGDTAFANKKGQSMVSDGTVAIFISLIMFIIPSKIPGLTEDPKKPGKLKAPPAILTWKTVNDKMPWNILILLGGGFALAKGSEESGLSKWLGDKLTPLQHVPPSATVLILSLLVAIFTECTSNVATTTLFLPILASMAQAICLHPLYVMLPCTLAASLAFMLPVATPPNAIVFSFGGLKVSDMARAGFLLNIIGVLTITLSINSWSIPIFKLDTFPTWAYSNTSQCLLNPPNSTVPGH

SEQ ID NO: 32 - DNA, 24910 bp, Mus musculus
gacaggctggtcctcaccatggccacctgctggcaggcactatgggcctatcgctcctacctgattgtgctatgtctgcccattttcctgttgcctctgccactcattgtccaaactaaggtaaggactggtgggtctcattcagtataagtgactcaaggggtgacgtcagggtcaaagtggggcatccttaggttttccttttcacctcccttccccagaaagtgaagccaccacctcctggtctttctgagagccaagggtggtttccgcagggcactttggagcctgctggtttgtggtcagaaagccaattgcataaaggaaatgcagccgacagatgagtttatctgactattgtgtgcccattagcgctgagctcagtgccagccagggacaggaagagccaagtgggtaactagcttttgagaggataagtaggagagagctggcctcaggcagctgtgccctcgaagataaaactaagataaatgactcacctgcagtctcacgggtggccactgagggggacccctttcagtgtgcacagccagagcacaaggtgcctgcctccttcccatctcaagaggttattcacttgaaaccccaccccccacccgcgcctgtgacgggcatcgagggattctgtgcttcatcttatgagccaggtcccactgtcccattttgcagatggggaaaaggaaggtaagtcagttattcaaagtcccaggacagaacttggtgccttggagcttggtttctatgtcctttgtggtttctctgtaagaaaattaacattaaatccgggtgtggtggcacacgcctttaaaataccagcactcagaagcagagacaggcagatctttgagttcaaggacagccctggctacgtagagagaccgtgtctggaggaggaggggcattatatgacaaatttaaaattagcttggaggtctggagatacaacccaatcaatggtaaggtttttgtctgtcttgtgcaagatctggggtttaataatccctagcaccatcaaaactaacaaaaacacagacaaaaaaaaaatccctaaattttagctaggttagtggtggtgcacaacttcaaacccaaaatttaggaggcagaggcaggtggatccctgtgaattctaggtcatcctggtctacagagtgaattccaggacagctagggctacatagagaaaccttgtctcgggggatgggggtgggggtggggggaatccctagatttgaatgaaagtatcctagagctttgggaaaggaagctccaagagggattcagacctcagcctcaaagtctagctgcctctgcaaaggacatggatgggtgggggcagaactagcggtgtggctgtctccaaaactgacactgaatcttggccatggaccatcccacgcccctggttctgcccaatgagccactacttgttgctacagataggggaaatcaatccaggaaggccttgggtttgcgttatctctgagggagaaggctcaggatacggtccccaacacttgcccagtgataaggccagactgattttggagataacctttctcagacaagtggcgtctctaagcgggggattgctcagatagatttcggacaggctggaacacgtgtgacaccaccaccatcctgggagaggcacacatagctcagaaagattcattgttttgccccggggtcacgtggcagagggaagctgagttgtttcgagaaagctgtgccttttggtgaaaggatggtgtgcagtggcgggagtgtgaagctccagggtttgctctgctgtacctggccctgccacctgcttctgcttgccctctgttgactcaatcaactgtcctgagaggtcaggccctcatttggaccttgggttcctatcctacacaagaatttgtggtcaatttctccttccctgcttgactgagcccatcaccatataaaccagattttatgcttctcatggcaaactgtcaagaccctgcaggtcacagtcactctttctctgagcctgccttccttttctataaccattcctttgctataaccatagcaagcctcagagtgtgcctgcacgcgcgtgcacacacacacacacacacacacatacacacacacgtgtgtgctttctttcctcactcctgacactgctatccagagtcccactagcttccagggtcctaacccaagacccatcctcaaccctgcagcaagatttgtcccagatggtcacacccactttgccctcacttgactttagtgacaccttctctgtctgagagacagtgactccttctctgtcagactcggcaggtttcccagcatccttcctgtcttcccccccccacacacacacccactccaacccctcctgattcctggccaggtaattgtatactgtctcataattggaaaagcatccctcttagctgggtgtggtatcactcacctctaatctcagcatttgggaggctgaagtagcaagatcatgagcccaaagccagccggggctaagtggtcagaacttgcctcaaaaacaaaacaagaacctccctgcccccaagaccttagtctaggatcctactccagacctctacagttgtatcagccaactgtcttgggtatttggtccacacaaatatttctctataatcagcctggcatccagatccaaaccaaagctcagcctggcacctcagaagacttttcacatcgtctcattgagaaatgtctcaatgcctttgatgctagccatgactccagccattctgaagggtccactttcccccacctactcacacaccagcagtccccgcagcacactcccaaagtgtctgcattatgatcctgtagtgccagggaagtgttagtgattcccccaaaacagacaggagccaatctggtgcaagtcaccgcagggtctttttattctattcgagctagcttgggctcccaggcacaccggccggcagtcacacaggatgattcttggtgagaagaggagcccgggatatctatcaggcaaggttttctagaaagcggcaagcaagggggtggggtgagtgtacatctaattggaaagctgctgtggcctttagcataattggctggtgctgggaatcaaatcataaacttaatctctagttccctctgtattggttagacaggaggtgggcttgtaacctgggggtgtaaacctgtaggggaataacctggagacgctggtcttgttgggggtcagtctagagactggagttaggttctggttttgttgtgggggtgtaacttggaaactaatgctaggtactgacctgttagtttacctgaattcaaacttaggtcaggttcttaaaaatggagtctgaacttaaaagatttggcctctcaatcctagtaaactcatcctaagttgagacttctataaatggaaaacacaccgtgcatccactaatggctacccactaaccacagcctggcgatgcagctcagggcaaaacccccgccgctgccgccccaccctccctaccccacccccgtcagatcacactagtcactccggcgttttatttacagagcagtccctgtttgaaaggatcctggctgaggctgaggatgtcattccatgggcagagagggtttgcctaacatacatgaggccctggatttactactctctagcactctgagcatggtggcacagtcctctaatcgcagcagttgggatgtggagacaggaggaccagaagttcagggtcatccttagttacacagtgaggttggagttagcctggtataaatgaggtcctgtcaaaaaaaaaaaaaaaaaaaaagtgtgtatttgtttactttcttcctaatctgtgaggctgggagtttgacttactctctggcccctaacatgggaaggagagcccaagccaaggttcagatctccataataaatcagttaacgtttactaaatgtgttttatgtgcttggcactgggccaagcaccataaagacttaattcagttgtttcccacaagagacgtctttgtttgtttgtttgtttgtttgtttgtctgagacaaggtttctctgtgtagccttggctgtcctggaactcactctgtagagcaggctggcctcgaactcagaaatccatctgcctctgcctcccgagtgctgggattaaaggtgtgtgccaccacgcccagccagagacctctttttgaccatgatgttactgccgctgcctccacttcatagacatggaaactgaggcccagaggggtgatgtaacttgcttccgatgccatctggaaaaagaagttgagacactaatctgatattagagtctcctcctgaagctatccagagaggcaaggaggtcccccatgactgatcccttcaaatcctcctccttcatgaccacccagccaagccctcctcttttctctcaccaatgcgtgtgacctcctggctctttcccagagtcaaaggacaaaatacagatgttttgcagaaaatgtagttagggtctagaggaagagacaggtgtttgtgccacacagatgtagcaagagggctggagcactcaggacaggcagtggctacctcaaaggagtcaagatagactgagtgtccaggtggaaccacagctgtgagaggcgacagacatcggaaaagcagacgacctgcaataaccattgtatgaaacgaatgacaggcgatttcatgaaatgctgtccctttggatggtgtcagctgatacagtacagtgtggcacttgcccagccttcaggcacgatggcagaaacttgtcacccttatgggtgaacctcgcactcggtgctgtgtgacagacaccatttatgagttctggagagatgagtgggggacttgtcatcctcggggggcagagggggacatagggacagatacaaaagattgtgtactgcccacgctgcagaagagttatggcaagtgttgacatatatgaactggggaggagctagctgcccttagtacagagctcaagtcaggcctcccagggagtggacagttaccctaagaccagagtggcagtaagagccagttagacccagctctgggtaaagaactgtccaggcagaacccgcagtaggtgcaaagtgccaacgcagggatgagcttggcgagttgacagggcaaatgccagtgctgcctgctcgactacagttccgtgtcagtgaagttcagaaggaaggggagaggacagctgatggcagccctgtgaagctggggaagaagtggggacttcatgcttcaggcaagaagggataaagagcacatccgggttcctataaatgtcagtcaaaaatgcaaaacctcttggaacctaaaaagatcctctgcaaaactcagtcatggctccatgttgtccgttaagaacagaaaagagcagaattctttaagcatggccacaggacacacagttctgtgcatcctcagacaagctactcctgtccccagagcagagagcacctatacaagggatggagactctctgtctactgaggcttcccgtctctaggagaagacagagaatagcacatggcttttggagccaggtaggtgacagagggttctcacccactccaggccgttatcacacatctgcaggccttccaatcaccaagggcctttgtgagaaggagatgcaaccccagcaccagggtttcccatcctagggttctataaacccccacacccttcccatctgcccctggctcctgcagagcacagaaggccttgggccctatgtggataccacacttccccacagctgttcagacgagttgttggccccaccagagctcttggatgcatggatgcttctatgaaatagagaggtgtatcttgtcaatagccccaaatggtgagtggtggtccccagcagagaagggaagctggaatgtacagagataggtttcatctagaagcaagatttgaaagagtcacccccgccccccagccaaagccaggagatcccttgaaatagagacagaaagtaccctcatgttgattttacctgctgattactaaatcgtgacctgactcataggcacccataggagcgtcgtgggaaaggaaggtagagtctttgatggtaaaacctgattacagtctacatggaaacctgggctactgggatgatccaatgcccagcaggctgtgggaatgccggttcgtgtcactcagcctgtttgagcccactctgcagcaactggagcaggcctggctccaggcacaaactgtgaagttaggagtcattctcaaaggtacttggtggactgaggctggaggattatttgagcctaagaggtcaagggcatcttggtaatttagtgagaccttgacaaggagagagggagggagaggaagttctctgacaaaggcatagctgtgttgaagattttgccaagggagtggttaaaatgttctaattgtgaagagttggaatgttgcagatcaagaattattgaattatctttagttcacttaatggccatgtggtacccggattgtatctgacctaacatctctgtgatggctcggaaaggttgatgggatgtattgctaaccttagcaatcctagtacctctactatgcctggcattgagaaagctatcagttagcatctaagtgagacccattgttgagatcttcaggtttggctgtagcctcacattgccagtccctagcaatgcattcagaaaacactttggtttctggctttcttccattctgaggtcaaaactgtgcttgtaccaggtggtggtggcacatgcctttagacccagccattgagagtcggaggccatccttgtctacagacaaccagagctttatggagaaacactttttttaaaaagtacacatagtttctcccaaactgcatctggccaaaggtgctcacgtttggatcaaattgtgtgtgtgtgtgtgtgtgtgtgtgtgtgagagagagagagagagagagagagggacagagagagagagagagagagagagagagcgctttgttttggaaggaagggaacgagatagggagccaggaccgactcttcctcggttctcagagatagtatgaacaagcagccattcccaacaggaaccaggtcacagcattcacccaaatgagaacaaaccaaggcaccagctgccatcactgtgtttggcagagcctcaaaggcaggtggaggctcttgaggctttaaaatagacaatgacttcaagtgttcctcactgggggctgttagcctagcaaggctctagaagaggaccactctgtaccattgctcgtggggatgagagttggctttccctggctggtcctcagttgaaagtagtagatgggaaagcacacactcctgctgcaagataaaggagctggggatggggtggggctcaggtagttatcatctatctgttattcctgggaaagatcacacagacacacacacactcacacacactcacacaccccacatacatacgtatacacagacatacatacatacacacaagtgcatatacactcaggcatgcacacactcatgcatgcgctctttctctctctttcacacacacacacacatacaaacacacacacattctctcttcatttcagtatgatttccttcttcacagtcctgtgctgttttattttgtatgttttaaaggcttagtctgaaaaaaatgcaagccttcttcagacagtgaaggggttcctggttcccaaccctgggtaagaaacccctatcagaggcccccctacaaatgtacaaatgctctcacgtgccagcatatcacctacatggccttccttctgtgcttcatcatctttctttctttcttgggctgcgaaggaacccagtgcctcttgtatgctaagcacagactccgctgtccagcatcaccccagccttcaagtaattgcttagttaaaacactatcctctctatatatctctctgcttcaatccttcaaaagccttccattttcactggcctatctactagagaaggttctagatctttctcgaggcagacaagattcttgtcaagtatacatctcttcaggcccctgcctgacctttctgcctcatttcccacccttctgtatctactctccacccgacctctgttgccgcagctgaactcctacactcccctcagctcccactcagcatctcctccctggaagtcttcctcgctccagcagtagggggctcagcaaagggcatctcctcttgggagcctcactcttctgggttcctttgcagtggtgcttaacaagttgccacctaaatgtcctagcttgcttgaaaccctgagaatatccttgtaccctggtagcagcatccccattttggggggctgaagggcagtgggagtggcctgctgtgagcctgtcatctccaccccaggttaaccttcaattacctggaggaatgacctagggcaccattgagactgtgggtgtttgctatcatctcagagctgtgctgtgctgtgcgtgtggttaaaggctatgctgtccatagccgttgtctgccctggcttctctggcactgtcatttccacgtgtgtctcatatatctgcctggttctcttgaaggccaacacttactttactctctgacaaaccccacccaccgttcaccaggtgcagcccatttccactcagccatacctgttagcccacttcctccctatcctatcctcaccaggatagatgtttcttttttttttcccatgaaggagacacatctgtgtacagctggaaaaaaaacaacctttcatattttttagggcattaaaaaaacccaagcgaggttaaggggattgtctgaggatgtagcaataagtggtttgtccaaggattcaccaaccattctgggtctgggcgctcaggttttgagcagatgtgttctttccacagtgaaataattgagcccccagccttggacgtggaggctcctgtaaggcattaccctccctgtttacttctggcttttcaggccagcccacgtagccagcatgcagagactctgggactccctgaaggccgagctgggcatgaatcacatcttacaaggcagtgcatctgctcactgtttttgtctgttcgtttttttagcagaagcagacttggacagtctatggtccaatgacaaagactgatactcatatatttcataaaactgaatgctgccaggcctggtggcgcacacatgtaatcccagcccttggaaggcagagattccatcatgatccatagagcgagcctcagtctcaaaaagaaaagatgtgggaggggctcccctgacaaaagaatacctggttgaagatctaggaatgagtgggcatccactgggcaaaatggggaggaagacaaagtccaaaaagaaggacttccgagcaaaggtctctgtgaccatgagtgaggacagagggcttgagggaatcatgggagagaatgtctggccaaactggaacagagcctgcaagagtgaaaagtatacggtacagggcacccagactccgtatggaactcagagaacttgggcagggcatgcctgccggcactgagctaccaaacaacaaagaattagttactaagccaagaataagttgcttcagtgttacaatccagaccgagagctgggagcagctcccctccaggtctgaagagggaggaggcagaaagagctcagtacaaaactcttctattgcatgaactctgggccctggttcaggctctgccctccccccaaaaggaaactgtgcccctgagatctgtgccacccagtggccctggggagctgccagaggcctgcacataagccagcagggctgcatgtcagggctgccagcccacgtccgcagttctcggctgtcagacaatgagtcttagtgaggccttcctgaagtccccatccctagagctggcggtgccgacctggagtcagacatagcgtggcacaggagagaaagtcaccccagtgccctgccctttggcagtgcctggacggtggcacctaaccacagaacctcacagaaccctggcagcacactagctccaagctggactttcctactttcaaaaagtgggtcaaggcattgccaggccacaggcattgcccagaatattacttgaaaatgtgtaaaacaaacaacgaagactggcagaagaaaacagttatgaatcatggtttccaggaaaaccccgctgaggtttagaaataacgtctttccttgctggtgccattttcaaacactatgctgaaagggatctggaggtgacaggcagaagccatgtgggcaggtggccagtatagctacaatccaatgtttgtaacagaaccaggccaggtggcatacgcctattgtcctagctactcctgaggctgaagtgagagggtcatttgagtcccagagtttgaggtcagcatggacaacttagtgaaaccttgtctcaaaaacaaaatatctataacaaagtcaccaaccaataacaacaatggggagctgagaagacagctcagtgactaaaagttgtgtaatggtgggttagattcccagtgtctgagttttaaaaatgagcgcagttggcacacacctgtaatcccaacacctgtgatcccaacacctgtgatcccaacaaacacctgtgttcccaacacctgtaatcccaacaaacacctgtgttcccaacacctgtaatcccaacacctgtaatcccaacacctgtgttcccaacacctgtgatcccaacacctgtgatcccaactgtgtggcactggagccaggaggattcctggaactttggagctggccagactagcctagtcagcaagattcagtgagagatcctatctcaaaaaactaaggtggagagcagttgagaaagatatcctatgtcgaatacacacatgcacatgaacacttgtacacatacacatgcacacacacattacacacacatacacacaaagtgacaataacacagatgagcctcagctccataatcctgtttttaaggttttatttctatgttctttatttctatgttaaaacagacaactttgctttacctacctcaaacatgcgcaaaacatgtcatttttctacatgggcaaacttgtccaaaacaaagcctacatttttgttgttggacattgagctattttataactatgggtgtgtttgtttatttattgtgtgtatgtgcatgtgtgtcatggtatatgtggaggacaacttgtgagagttggttttctctgagttctaggttttgcactcacattgcagccttgacggcagtgcctttatccactgggccatctcatctgtctggccctgcttctttggttggtttcagtttttgctgtgagtaaaggtctcatgtagcccaggttggcctgaaacaccctatgttgctgaatttggccttgaaatcctgacccttctgcctcccacccaagtcctaggcttacacatatatcacgatactacacaaagcctactttataatcaagtgttgaacatcgctttttagtcactgactccaaagtgaaagacagacaaagtatggcaagatacggaaagctgtggaaggtgtggctaagtgtggcaaagtatggtaagatatggtaagatgtggaaggtagggtaaggcatggtaaggtgtggtaagtaagatggggtaaggtatggtaagatgtggcaaggtatggcaaatatgttaaggtgtgataaggtatagtaaggtgtggtaacgtatggttgccaaggaatggtaagctatgataatgtgtagtaagttatggtttctggtgaatgcttctttgtgtaaacatgatgtgccaggctcatagtgagccttctgtgagccagggcagtctgtgctgacttcttcctcagctgtgctcatagccttcactagacacatctaaaggaacagtttagttcttccatcgaagttcacaggtcccaggatttcatgtcccagctgaaaatctatagtccaggaaagacaaatgagcaacagtcactatgctagggagatggaatggggacacaggaccaaagccatcagggtcaaggtggctagatgggatgttgagacacattggaggagctaggcatagctatcacgtgggtaaggataggggccccacgctagacatacaggacctgtagtctctccaggcagctgcattgactgtgataatggggtctcaaagatgaagcaccttgcctcggtgtgggtggcacacgccagaagacttaggcaggagagtggcctgatccccagagtttgcctcagcctcccagatgctaatcacaggcatgagcccctgagtctcactgggcagccagcctcacctaagaggtgagctgtaggacagcgagaaaatctggctgactgctcctgcggaacaccacctgatgttctcacacacacacacacacacacacacacacacacacacacacacacacatacacacacagcaaagccccttatacacatacacatacacacacagcaaagccccttggtgaatctgggcagtaggcagtgccaggccctgggaaggtggttgtttggcttcagaactccagttctctccttgtccagtcccagggagcctgagccccttgctgaagggaagacttgagcagaggaaggtaaatgcagagctgaggagccaggtggcctggtttgccacttactattggtgggactttgatagttgatctaatcaatctacacctttttccttttttctgtgaaccgggggatggggaggggatggggggaaccagggcatttgggacctcaaggaacaactgaatgtgactgaagagcattcaaaacccacaggacacaaggcctggccaggaggcagcttgggatacctggcaaagctgtcttctggaagcaacagtgttgacctccaacctcctgtctttgaatgtgcagttctggggtttggggcacaagggtccagagtgggtggcctgcccttagtcctaggggtgggtaaggagttgtctgtccagccagggcaagtacagaggaacaagaaaaaaactggcatcagcagaaaccagctggttgtggaagcaggaggatctctggtcaagggtcataggggccctacccctgagttgatgaattcaggcccgggactggaaagggtagaggttgagtgtgttcaatcaattcttgacatcactgaatgaaatgagagcttgcccagagacagccaagactctgcagctccaggaacaccttcctatctatttccaagggcgtgttctctggagcacatgggcttacacagtcctgtctctcaacctagtgcctgtcctaactcaaccactccctagttctcaagaccttgagcaagccactcgcttccggggcacatccgtaaagggagaccaccacacccaccttaatggtgcttacattttcaacccagaagtcctaaaagttgctcaataacagtttcctcttccctggagcctctcctcctcactctccaagaaactctcctcatcagagtactaaggctgttgggtataaggagaaagcaaacaattaaacggataagtgtcccagcccacagcctacaaaacccctgcccgccattcctttctcagcaaacagtctgggcaggcgtccctacccaggggcgtccctctgggtaatcattccctggtcctgggagaggccctgccagggaggctggacccctgatgtcttgagcccatgtctggcttctgactattctaggcttaaggtcaggaactgtgggccagaagatggaaataaagaatttctggtacctcaaatctgtgcctcagagtagccccaaatctcttacataatgcaaggcttggaagaggctcccacatactctgtaaaagtgaactgtgccttccattgtatgctgagtggcccaagcaggagatctatctcaaagaattcctggggattcctttcctcagacaagattagcttcaaaactgagacccatagatcctgggctccagggatcgcagatgccaccaagctgactctgtcctgggttaggacttagtggcagaagcctttcccagcatgcctaagggcctaagttccacccccaccccagaaccaccaactaaagcaacatatgagaaggatcagagtcctgagcagggcctgattagtcttaagctaaatgagacttgttacaatgtcttagctccactgggctgtggtggcacatgtctttaatctcaggactcagaaggcaaagacaggcagatctctgtgacttcaaggccagtctggtctccagagttccaggatggctaggtttgttacacaaagaaaccctgtctcaaataaaacaaaaacaaattaaaaaaaaaaaaaaaagtcttgctgggcagaggtggcacaattccagcacttgggaggaaaaggcagggggatttctgagttggaggccagcctggtctacagagtgaattccaggacagccagagctacacagagaaacactgtctcgaaaaacaaaacaaaacaaaacaaaacaaaacaaaacaaaacaaaacaaaaaaagtctcggccctaaggtcaaagacaagaggagaacaccattttttaagtttgggacagggttttactggccacataaaccatcattaatccctgcacggtagatctctgtgttaaggacaaccttgtctacatagtaagctccaagctagccagggctctgtttcaaaacaaacaacaaaaataaaaaaaaaaataaaaacacagaacttaagataatgtttcatgcatagcccaggctgtcttcaagcatgctatgtagtcaagggtgactttggactcctgactgcctgtgtctacctcccaaaggctgggcttacaggctgcatggtgcttgggagtgtatatgaggcactatgcatactaggaacactctacctctgagccatagccccagctcttcatcgtttgtttccaccctgagatgggaccttggacttgcatcaagtgttcacaagctctcctcatgctggtgcaaatggccaagtctgttctctggggggggggggggggcagaagggattgtagctgcgctgtccaactgctctgcttccctcctcctgcccacaggaagcctactgtgcttactccatcatcctcatggcgctgctgtggtgtacagaggccctgcccttggctgtcaccgccctcttccccatcatcctcttccctttgatgggtatcatggaagcctccaaggtgagcacccccatccacagagggaaagactacagggaaaaggaaaatcctccctagagccttggaaccaaagaagcctcatcacaaggtcatggaagaaggcagagggcatgagagaggatcagtcaggaggcagagagtatcctagaaacgtgtggctgttgtgagagttcattcacagtaggagggatgagccctgccagagaggtgcaaggccacctgcaacaagaggaggcctatgacagccaactaaggatctcacccaacacctggtcctggtagcacattattgtaaggctggcaccaagaggctgagatgggaagatcttaagttcaaggccaacctgggctacgtaggtagatcctgtcaaaaacaaacaaaaagatcaactgttctttctatctatctatctatctatctatctatctatctatctatctatctatctatctcttggtctccacaaagccaaagtgtgctctaaaacgccctttggattccaagatcccaggatgctcaattctcttacataaaatgtatagaggttttgtagtgtgagggcacatcttccttgtgatacctaataagtagtttctatactgtattggaaatagggctgggagatggctcaggggataaaggcgcttgctgtcaagctagacatcctgagttcagtccctgggatccacacaggagaagaagaagacaaccctgcaagtcatctttggacctccacacttgagccataatacatgtacacacacacacacacacacacacacacacacaacacatgcacacactcaaaacatgcatgcaaataagtgtaagactttaaataatgatgcacaggagagtctgtacccttcagaataaatgcaatttcttcctatttccttttttttgtttgttttgttttgttctttcgagacagggtttctctgtgtagcctagatgtcctggaactcactctgtagaccaggccagcctcgaactcagaaatccacctgcctctgcctcccaagggctgggattaaaggtgtgcaccaccaccgcccagtgcctgactattttctacacgaggttacctcatccatgaatgcagacgccacatacaaaaatctaatagaggactgactgcacattctattaaaaagatgtttgtatttgatgcacacatttatgtgtatcacatttgtgcctggtaccctcagagatcacaagagggcattgaatgcccttgtgttaaagatggttgtgaaccatccagggacactgggaactgaacccgggccctctggaagagcagaaagtgctctgaactgatgaaccatctctctggccctccagctatacttctaatgtaatggctcttgagtttgaatcgcaccagaacaaccagaactgagaagcaaaccagatagatgggccttcatctgagctcacctaaatctaaaggattttcctctttctcttcccttcctctttcctcttcttcctccttctctctcttcctcctccctcccttcttccttctcttagtcctcttccttctccttcattctaacacagcttctttctgtgtagcccaggctggcctcgaattcacagcttccctgctccggggacttcagctgagctaccaagccccgctgaacatttggcttgtaaccggtctcaggggatggtgttgatactggcccagggatgacacatacgttctagaaagagcagagaattagttatctcccagttctcagcttgaaccacaactggactcaggctagagcctcccagtctttgagaggagaagaggagtgagtttaggaggagcaggcaacctggctcctgtcaacggtcggtggcccaaaggccatttccatttggttctgaggtcccaagggaatgccaatttgtaacgatgtcatgactgttccccacaagctcggtggtatcaggaggagaccccagagtctcagacacatacctgctctcccccaggtctgcttagagtacttcaaggacaccaacatattgttcgtcgggggtctgatggtggccatcgccgtggagcactggaacctgcacaagcgcattgcccttggagtgctccttatcataggagtgcggcccgccctgtgagtccgtgctagccaggtgggaggaacaaacgggcatcctatgtctggaagacagctggagtggcagtgtgtctgtctgtttggttctaggctgcttctgggcttcatgttggtcacagccttcctctccatgtggatcagcaacacagccaccacagccatgatgctgcccatcgggtatgcagtcctggagcagctgcagggctcacaaaaggatgtggaggaaggcaatagtaacccttcctttgagctccaggaagcaagtccccagaaggaggagaccaagcttggtaagagacatgaaacagactctatcctcagcccctccatgcttgggaaaacctaacatgactacaaaagtattcatcttggagcccatgggggagagagagagagagagagatgagtggccaacctgtccaggtctttttgaggctttcctagatttaaaatgccagtggaggttggtgagatatttcaggaggtaagggtacctgccagcaagcccaaagacctgagttcgatccctgggatccacatgatagaaagagggaactggctcctgcaagttgtcttcagactttcatatgtgtactgaggcacgtgcattcgcacactcgtgtgtgtgcgtgcgtgtgtgtgccagtgtgtgtgtgtgtgcatgtgcatgcatgtgtgtaataataataataataataataataataaatagttattattgttattactaacaacaataataacaccataataattttgtttcaaaaatattaaatgccaagatccatggggctaaaaatggtagggcagttgcgcagcatgttgagtcctggtaatgtaacaatgataacaatagtaaacaccaaagagcgaagcaccctgtgctaggacccttcattcccagcaatccaagagtttggtgagaagcgagctaaagtctgggacatgggacgtgttctgggctttattcctcctgcttggtctaggtaaccctcttcacatctctgtgcttcagttccccaagacacgttgaaccagaacgtgtctgagaagcccacagctccagagaatgctggaatctggagacagcctctgcccacggcccctggaggcctcaggacaaagtcaggactcctgcagttactcttgtccctgtccccacatgcttttccttgttcacagataacggtcaggctgtatctgtttcttcggagccaagagctcagaagaccaaagagcatcaccgcttcagccagggcctgagtctctgcatctgctactcagccagcattgggggcattgccaccctgacgggtaccacacccaacctggtgctccaaggccaggtcaactcgtgagtgccagcggtgggggcagcatgacaaaagggggcttgcactctcactgagtgacaaatgggaccaggcagttttagggtcactgttacacaacagcccatgtcttcttcatcaggatcttccctgaaaatagtaacgtggtgaactttgcttcatggtttggttttgccttccccaccatggtgatcttgctgctactggcttggctatggctacaggtcctcttcctgggtgtcaagtaagtggtgccttaggtaagagaagcccagtttcctgcccatacccctgggagcctctgtttcattcaacgtggtgggggaccatgctgcaagtaaaccttccctctctttaggttcttggcctcccacatgagagaattactgggtgcagaatgctgcccctgttaacagacaggcaagatgagtctgatggaatggtgaattctgggatggaaaaccatgccgtgtgtgtgtgtgtgtgtgtgtgtgtgtgtgtgtgtgtgaggggggtccccttattttctgagacaaggtctctcaataaatctggaagtcatctggattgctctggctagccagtgaaccctagggctcctttagtctctgcatccctacagccattcttatatgagtgctgggactgaactcagatctggatgttcacatggcaagtatcttaccaagtaaaccatctctccagccctgaagaccccagccaggcaccttcccatagtgctccatggtccttggatgagctgttggggattcctcaaggaacacagtttgttagagaagcatttgaggtgattcttctgtttgcctatgcacgcatgcatgccctagacactcggactcccagaccttaggcaaatccccttccttgctgtacttcatctgacgggtgatgatatgacgggagctgtaagaaaagcaagcacagggagtggccatgtcagaacgttcctggacatggtatcagtcttgcaggactgatagccaggtatagaaatgcaagtaggcatccaggcaggacacgcacagagcatagaggccagaatgacctgggtgtgtcttggcagcctgcttctcgagagctatagtacaaagaagccgttggggaacagtcacagggaactttgtgtgtcacatagagtcagacccttgtccttgaggccaagatgaggagataaatttcaaccctgagttactcctcattacagtttggagatctatgttgagatctatgtagccggatgtggcggtacatacctgtcatctctatacctggggagctgaggcaggaggattacgagttcaaggccaatctcagttacatagcaagactctatcttacaaacctaaaatggagtaaagtagtcatatgccctgctaaggctcacttgaactggagtctgtaatctattattgatgtctcctgggcacatagattggattggtagtctgagtgttactggggtgtgaattgtggttattcgtccatcctttctaggcagagaagagtcgtagcaagctgtttagctgtactttgaacaaatcacttgggaaggtacaggaggctgggttagggttggaagctgctacagggcaagggagtagctgatgaagtcagcacgctgttggatacagtgaggagtcacagatgtgtgaaacatgctctttgcccccaagaaactcacagaccagcaagagataagatttactcaagtgtaaagtcaaatgccaatgttaagaaacagcatgaggaggcagccaaagccttctaatggctaggactaggaaggtacatctgagcctggtaggagttcctggaatactaatgaaaagaggactcagcatcaggacctgaagtcacttggggtctggaccactcatccttctcaccttggacaggaagggcttgacacaagagactcttcagaggaagggtcacggtggggaagagaggaagatataactttcttgttaaagaaaagctacaatcccaagccaggcatggtggtacacacctataacccaggtactcaggaatctgaggcaggaggatcaaaagatggagaacagcttaggctaaaaacaacaaaacaacggcttagtggagagcttacccggtatgcaagaaaccctgggtttggtcctacataatctgtgcatgataatggatgcctgggattcaaggtcagcctgggttatatgagactctgtctggagtgggggagccaggccatgatgcccttctgggtctaggactcttttctagagccaggacattagggcttgcccatgcctggtgcagagcctttatacactgtccctgcctctgcccacagcttccggaagaactttggctttggggaaggggaagaggaacggaagcaggctgccttccaggtcatcaagacccagcacaggctgctgggccccatgagttttgcagagaaggctgtcactttcctgtttgtcctgctagtggtgctctggttcacgagggagccgggcttcttcccaggctggggtgacacagctttcgccaataaaaaagggcaaaggtgagaccttccaaagcaggagaggggaaggcctgagggactgcacggagagcaggaaaaggaaggaagaaggcgggaagggagttagggaagagacagggtgtctctctcgggggcaggctgatgggagggacccttctctgtcccctactcagccttccttcccaccctttccacagcatggtatcagatgggacagtggccatctttatcagcctgattatgttcatcataccctccaagattccaggactaaccgaggacccaagtaagcacctggcctggggctgggaagggtctccggcagcccctgtgtgcaggtatatagcctttgaccacatttggcaggagccagcaaagctcctttgggtgacctgctaaagacacagggactcaaggacagagttggaatcctacagagatgggagactgagggctccctctacctaccttaagctctgataggaacccgcttaggaaggaggggaggggaggcctattcacttttggtgatcatccttgtctgcttggggagtagaaaaaccagggaagctgaaggctcctcctgccatcctcacctggaagacagtgaacgataagatgccctggaatatcctgatcttgctgggtgggggctttgccctggccaaaggcagtgaggtgagagtctgtctgagggagatcctgggatagtcccaccctaggcttaagcacctttccccgaggtgggttcttgaagcccaaaggagagtagtcctctcagcgatgagcctcacccccacactacccacatactcctgaagtcagtgcaatgagcctaaccaccaaagctagcaacagtttgatgcagttccagaggaagcctccagaggacgccagataacagtctttcagggaccgaagcgtctttctcttgactttccatccaaaaaagagctgaaattccagatactgtagccccggacctccaggtcctagcagagatgagctaagggttgatataggagatttctgggtctgtgcttagcgtatcgacagtctgggccaggcactttgtttggtgtggagacacaggaacacagtgcctggaaacagcattagcagcaggtcccacagcctgacatgacagtgggtgaggccagaccaggagctcttcctaccctgggatttgcccattttcccaatagcctcttcccagcctcttcaaggccctgctccccctccctgtcaggagcacagccccttaagccctaatactcagcagggagaggactcaggtgatccagggtctccaaggagtcaagaaagtaaggtgattacacccacaaatagccaggttgcccactaggacctccccctcctcatcccactggtctcaaggaacaggaggaagcttccttggttccagctgggtaccaacttccttcatagctgtttgacttaaaggccacagaggtgtgggcagggatagggtttcccagagttccagctgagaacctgaggaccccagacagttctgtctcatatatatcacctccttctggcttcaggaatcaggtttgtctaagtggctgggagacaaactgaccccgctgcagcacgtaccgccatcagccaccgtgctcatcctctctctattggtggccatcttcactgagtgcaccagcaacgtggccaccactacactatttctgcccatcctggcctccatggtgagctgacactgaagcttcctccaggcagccctcctgtttgctctctatcccctactggtcagagtggttggtctaaacctctccgtgaagagaacctcctaagtgtctccccttttctccagatgttgaactcttaggtcctaaggccttctggcccctgagtcacctgtttcaggtgacatcattctttgagtatcccagtggggctgactggaggcctcacccacctccccacccaatcccctgttctggtcattccagaagtgatgggggtaaaattgctccctggagcgtccccagccccttcctcctcaggcgtgttcccacagcctttgatgacacctcctgagcccctttgagatcagttttatgagcccctcccatcactgcaccccacagggcagggcagcacagtcatctcttctgagaggtcaaggggccctctccaggtcacccagagcgctctgcccagggactgagttcatgacaagagagaaaaggggtggggcaggcccccaaaagggagaggcagaagaactggggtctagccttcccactgagcctgaattgggatcaattatgtccactgtcactctccaggcacaggccatctgccttcacccgctctatgtcatgcttccctgcaccctggctgcctccctagctttcatgctacccgtggccactccacccaacgccattgtcttctcttttggaggcctcaaagtgtctgatatggtgagtggcggggaagtcagaacgcctcagcctttcacctgcctgagggtcactgaggtttccagaaaagagctggccctggtgcgtgggggtggggggcagctctgacatctttatgttctacagtaccaagtcacaaataaggggaggggaacatttggaaggtgtgggcaggggagggggggtgcagcatccccacttaatgaaatgacctaagagaaaacaaggcccagaaccccaggaatgtataaggaacccccactttccctgttaggatcagtcaaataagagacaaagacattgcctaacactgcaggttagactgaacctgaaggcaggacttcccaaggagggctttgctgatgggaggggttctgagtagccctgacaattgaggaatgtgtctctgattcacattggcttgcctcatcctaggcccgtgcaggattcctgctcaatatcatcggagtgctgactatcacattatccataaacagctggagtatccctatcttcaagctggacacatttcccacctgggcctactccaacacaagccagtgccttctcaatccgcctaattccactgtaccaggccactagactatggtacaacctagctctgctaggggaaaagccgctggtgccactcctcggagccaggagagggcgctccaaactccaagtcccgggacagaggtgcagaatcggctggcgcatgcgaaatcatgtattcgtgtgtttgcctacatcctgtatgtgtgctctccggtgaagaggaagatgctcgtgtctccgtgtggtgtgcgtggacgcttgcgtgtggtgtggcctgagggctcctcagtagttgtatgcatggcaaccgcgcccgccctctgactcccagctaggcctccagatctctttgccctgtatttattgaaactctgggcaggaggcccctgggagcaggaactccaaggttcattaaaggctttttcagtagagtccgggtgtgtttcccggtgttcag

SEQ ID NO: 33 - protein, 441 aa, Streptococcus bovis
MEKKLPATAANETDWRNKLTKTRIGSVTLPVYLVTASIILVTALLEQLPVNMLGGFAVILTMGWLLGTIGGNIPILKHFGGPAILSLLVPSIMVFFNLLNQNVLDSTDILMKQANFLYFYIACLVCGSILGMNRKILVQGLMRMIVPMALGMILAMGVGTLVGTLLGLGWKHSLFYIVTPVLAGGIGEGILPLSLGYSAITGLPSEQLVGQLIPATIIGNFFAIMCSGLLSRLGEKRPELSGQGQLIKITNSDDLSDALEEDKAPIDVKLMGAGVLIACTLFITGGLLQHLTGFPGPVLMIVVAAFLKYLNVVPKETQRGSKQLYKFISGNFTFPLMVGLGMLYIPLKDVVGMLSWQYFVVVISVVFTVIATGFFVSRFMNMNPVEAAIVSACQSGMGGTGDVAILSTANRMTLMPFAQVATRLGGAITVITMTAIFRMLF

SEQ ID NO: 34 - DNA, 1326 bp, Streptococcus bovis
atggaaaagaaattgcccgcaacagctgctaatgaaacagattggcgcaacaaattaaccaaaacgaggattggttcggttacgttacctgtttatcttgtaacagctagcattattcttgttacggctctattagaacaactaccagtcaatatgttaggtggttttgccgtaattttaacaatgggttggttacttggaacaattggtggaaatattcctattttgaaacattttggcggtcccgcaattttatctttattagtaccatcaattatggtctttttcaatttactcaaccaaaatgtcttagactcaacagatattttaatgaaacaagcaaactttctctatttctatattgcttgtttagtttgtggaagtatccttggaatgaatcgcaaaattttagtccaaggtttaatgcgcatgattgtcccgatggcattaggaatgattttagcaatgggcgttgggacccttgttggaacactgttaggtctcggttggaaacatagccttttctatatcgtcactcctgttttagctggtggaatcggtgaaggtattcttcctttatcccttggttatagtgccattactgggttgccaagtgaacaattagtggggcaattaattcccgcaaccattattggtaatttttttgctatcatgtgttcaggtttacttagtcgtttaggagaaaaacgccctgaactttctggacaaggtcagttgattaaaattaccaattcagatgatttaagtgatgctttagaagaagacaaagcaccaattgatgtaaaattaatgggcgctggtgttttaattgcatgtaccttatttattacaggcggtttattgcaacacttaacaggctttcctggtcctgttttgatgattgttgtcgcagcctttttaaaatacttaaacgtagtaccgaaagaaacccaacgcggatcaaaacagctttataaatttatctctggcaattttacgttcccgttaatggtcggcttaggtatgttatatattcctttaaaagatgtcgtaggaatgcttagctggcaatattttgttgtggtaattagtgtcgttttcactgttattgctacaggatttttcgtttcacgcttcatgaatatgaatcccgtcgaggctgccattgtctccgcttgtcagagtggtatgggtgggactggcgatgtggctattttaagtacagccaatcgaatgaccttaatgccgtttgcacaagttgcgacacgtttaggcggtgccattacagtaataacaatgactgctatttttagaatgcttttttaa

SEQ ID NO: 35 - protein, 540 aa, Arabidopsis thaliana
MNGGDVTVAGSDDLKSPLLPVVHNDEPFERQTVGQQLRTIFTPKNCYIALGPLLCAVVCLCVDLGGDETTTARNMLGVLVWMFAWWLTEAVPMPITSMTPLFLFPLFGISAADDVANSYMDDVISLVLGSFILALAVEHYNIHRRLALNITLVFCVEPLNAPLLLLGICATTAFVSMWMHNVAAAVMMMPVATGILQRLPSSSSTTEVVHPAVGKFSRAVVLGVIYSAAVGGMSTLTGTGVNLILVGMWKSYFPEADPISFSQWFFFGFPLALCIFVVLWCVLCVMYCPKGAGQALSPYLHKSHLRRELDLLGPMNFAEKMVLAVFGGLVVLWMTRNITDDIPGWGRIFAGRAGDGTVSVMMATLLFIIPSNIKKGEKLMDWNKCKKLPWNIVLLLGAGFAIADGVRTSGLAEVLSKGLVFLETAPYWAIAPTVCLIAATITEFTSNNATTTLLVPLLIEIAKNMGIHPLLLMVPGAIGAQFAFLLPTGTPSNVVGFTTGHIEIKDMIKTGLPLKIAGTIFLSILMPTLGAYVFASMGGV

SEQ ID NO: 36 - DNA, 3097 bp, Arabidopsis thaliana
aacacattataataaaagaaataaataaatttataaaaaggctcatttctcttcttctttggttcttacatttctctatatcattttttgtccttccattaaaaacggaataaacagaatgaacggtggtgatgtcaccgtcgccggatccgacgatctgaaatctccgctcttaccggtcgtccataatgatgaaccattcgaacgacaaacggtgggacaacaacttcggacaatattcacgcccaaaaactgttacatcgctcttggtcctcttctttgcgccgtcgtgtgtctatgcgtagatctcgggggagatgaaacgacgacggcgaggaacatgctcggtgttttggtgtggatgttcgcgtggtggttgacggaggccgttccgatgccgatcacctccatgacgccgctcttcctcttccctctctttggtatatcggctgcggatgacgtagcaaattcttacatggatgacgtcatctcccttgtactcggcagcttcatacttgctctcgccgtcgaacactacaacatccatcgccggcttgcccttaacgttagtctttcctttatctctttatcttcttcttcttcttcttcttttttctacataatttattcaatatttgattctttccgattgtgtgcgggtccacgggtgaattaattacaaaatatgatcgttaaataggtggattgcttcaattgaatccctacttataaaattaaacattacaatgtgtagtggtccatgttttatacgtggtgatgccagattatatgtttttccttccccatacatttttttccccacacacatttatttcgttttaattcttcttcaataatttgtgtgttcaaagtctaatgataacttgatcatttaaagttaagttattaacatacaaagagtcctttatttcaattttttaaacaaaatctttttcatatgcattttattttattttatttgtatcatagatgagatgtcatttcatgaggccctaggccaatccataattccatttatatgacaacccaaaaaaaaagatgagagacacgtaaacactatgtttctccagcaaaagcaatgatagtgagtatgatttaatttgtgcaagtgcgggctccaataattagctcacaagtcacaactataatcattgtttacttttatcatgtttttaaactataattaatcgtacgagagatatcctatgattaagagaaaactcaccagtcaccacgtatctaattatgataagctaataccataacttaatggataatctaattcttgaagataacgttggtgttctgcgtggagccgctaaacgcgccgctacttctgcttggcatctgcgccacaacagcgttcgtaagcatgtggatgcataacgtagcagctgcagtcatgatgatgcctgtcgctacggggatacttcagagactgccctcgtcatcatcgacgacggaagtggtgcatccggccgtggggaagtttagcagggcggtggtgttaggtgtgatatactctgcggcggttggtgggatgagcacactcactggaacaggagtgaatctgattcttgttggaatgtggaaaagctacttccctgaggctgatccgatcagttttagccaatggttcttctttggcttcccattggctttgtgcatattcgtggtgctttggtgtgttctttgtgttatgtattgtcctaaaggagcaggacaagctctctctccttatctccataaatctcatctcaggagagagcttgatttgttagggccaatgaacttcgctgagaagatggtcttagccgtgtttggtgggttggttgtgttgtggatgacaaggaatataactgatgatatccccggctggggtcggatcttcgccggccgagccggggatggaaccgtgagtgtgatgatggcgacattgttgtttatcatcccaagcaatattaaaaaaggagagaagctaatggactggaacaaatgcaagaaactaccatggaacattgtcttgttattgggagctggattcgctatagccgatggagtacgcaccagtggcttagctgaggtcttatccaaaggacttgtcttccttgaaacggctccttactgggccattgctccaacggtttgcctcatcgctgctaccatcacagagttcacttcaaacaacgcaaccactacattgttggttccgttactcatcgaaatcgcaaagaacatggggatccatcctttgctcttgatggttccaggagccattggggctcagttcgctttcttgcttcccacgggaacaccctctaacgtcgttgggttcacgacagggcatatagagatcaaagacatgatcaagacaggcttgcctctcaagatcgctggaaccatctttctctccatcttgatgcccactcttggtaatttactcaaaaccttcttctttatataataagattcattgtagtttagttttcttatagatattgttacatgatgccaggggcttacgtttttgcatcaatgggaggagtttagttctacaagccaagcacaagaccatgtacacaattgatttattgtactatattgtttactaccgtaatacatatgtagatatatagtcaatggaatgggaaatttagatgaggtcaaaattcacaaaatcggcccatttttgtaacccgcccgattatagtttccggtaccattgctttcttttgtgcttccaacgtaccggtaactttggtccctttttttttttgtttggtgtgtatatgaaaaagggttaatacagatttgtgtgttcttcttcctaacaatagcaactagtggcaaatccagcgcgcggtccaacctggccataccataccacgtcattgtccagcgtgtcttcttctgttttcggttttctacaaacacaaaaagcaaagaaaacaaaacatcaatgcatggtttataacgtcttgctttttaattagtttttgtcctttacactcatttatgttctttctttgtcctttgctatacactacttgtctattgaataatatgcttataatcg

SEQ ID NO: 37 - protein, 600 aa, Rattus norvegicus
MAALAALAKKVWSARRLLVLLLVPLALLPILFALPPKEGRCLYVILLMAVYWCTEALPLSVTALLPIILFPFMGILPSSKVCPQYFLDTNFLFLSGLIMASAIEERNLHRRIALKVLMLVGVQPARLILGMMVTTSFLSMWLSNTASTAMMLPIASAILKSLFGQRDTRKDLPREGEDSTAAVRGNGLRTVPTEMQFLASSEGGHAEDVEAPLELPDDSKEEEHRRNIWKGFLISIPYSASIGGTATLTGTAPNLILLGQLKSFFPQCDVVNFGSWFIFAFPLMLLFLLVGWLWISFLYGGMSWRGWRKKNSKLQDVAEDKAKAVIQEEFQNLGPIKFAEQAVFILFCLFAILLFSRDPKFIPGWASLFAPGFVSDAVTGVAIVTILFFFPSQKPSLKWWFDFKAPNSETEPLLSWKKAQETVPWNIILLLGGGFAMAKGCEESGLSAWIGGQLHPLEHVPPLLAVLLITVVIAFFTEFASNTATIIIFLPVLAELAIRLHVHPLYLMIPGTVSCSYAFMLPVSTPPNSIAFSTGHLLVKDMVRTGLLMNLMGVLLLSLAMNTWAQAIFQLGTFPDWANTHAANVTALPPALTNNTVQTL

SEQ ID NO: 38 - DNA, 3206 bp, Rattus norvegicus
ggccgcccggtctccggcgatcgcgcggatggcggcgctggcggcgctggccaagaaggtgtggagcgcgcggcgcctgctggtgctgctgctggtgccgctggctctgctgcccattctcttcgccctgccgcccaaggaaggccgttgcctgtatgtcatcttgctcatggcggtgtattggtgcacagaggccctgcccctgtcagtgacggctcttctgcccatcatcctcttccccttcatgggtattctaccctccagcaaggtctgtccccagtacttcctcgacaccaacttcctcttcctcagcggcctgatcatggccagtgccattgaggaacggaacttgcaccggagaatcgccctcaaggtcctcatgctggttggggtccagcctgcaaggctcatcctggggatgatggtgaccacgtcattcctgtctatgtggctgagcaacacggcttccaccgcaatgatgctgcccatcgccagtgccatcctcaagagcctctttggccagcgcgacgctcggaaggaccttccccgggaaggcgaggacagcacagctgctgtgcggggaaatggacttcgaacagtgcccacggagatgcagtttctcgccagttcagaaggaggccacgctgaggatgtagaggccccactggagttgcctgatgactccaaggaggaggaacatcgcaggaacatctggaagggcttcctcatttccattccctactcagccagcatcgggggcaccgccaccctcacaggcacagcccccaacctcatcctgctcggccagctcaagagtttctttccacagtgtgatgtggtaaattttggctcctggttcatctttgccttccctctcatgctgctgttcctactggtgggctggctctggatctccttcctctacggtggaatgagctggaggggctggaggaagaagaactcgaagttacgagacgttgcagaggataaggctaaagctgtgattcaggaggagttccagaacctagggcccatcaagtttgctgaacaggctgtcttcatcttgttctgcttgtttgccatcctcctcttctcccgggacccgaagtttatccctggctgggccagcctcttcgcccctgggtttgtttcagatgctgtcaccggtgtggccattgtcaccatcctgttcttcttcccttcccagaagccctcactcaagtggtggtttgacttcaaagctcccaactcggagacagagcccctgctgagctggaagaaggcccaggagacagtgccctggaatatcatccttctcctgggaggtggctttgccatggccaaaggctgtgaggagtcggggctgtctgcgtggatcggtgggcagctgcaccccctagagcatgttcccccactgctggctgtgctactcatcactgtggtcatcgccttcttcacagagttcgccagcaacacggccaccatcatcatcttcctgcctgtcctggcagagctggccatccgactgcacgtgcaccccttgtacctgatgatcccgggcacggtcagctgttcctacgccttcatgctgccggtctcgacgccccccaactctattgccttctccactggacacttgctggtcaaagacatggtgcggaccggccttctgatgaacctgatgggtgtcctgctgctcagcctggccatgaacacctgggcacaggccatcttccagctgggcaccttcccagactgggccaacacccacgctgccaatgtgaccgcactgccacccgccttgaccaacaacacagttcaaaccctctgaacactgatggggacttctttttccggctgggcgttcctcccagcgggttgttgctgttgttgctgctgggatcctacaagctgatcgagtaattcttccctgtaatctgctaggaggctgccagccaggttccctgggccacaggctcactgtctgcagcgccttctctttctttctcatgcatttcaaagctaactcctgcacctgatgcctgaggaacaggcttttctcaccgagctggtctgtggccacggtgtggggaaagtccacttgagccacaagctgaaatggcgaggctgaagtggtgtttgttttgcaacacctagggtcgagggtatcgagacaggaggagctatgtgactgcaaagctccagatgttacagatgttcacagctggctggattctgccttttctgtttaaccatctcccttgcagatgatacctggcagctagaggtcggcttccattgcctgaggcggagggagtacacaggtcttcctggagtctctctgctgcttccccaatctcgcaagcagcacaccatggggtttgaaaaactccaactcacacatctatccaagatgcctgggattctctttttttcatctgattctcttaggaccaagctctaggtcagccttgctcattatccttctagggccctcctgtctgtggccctggggaggggctctgtggctgcagaccaccagctgtttttactctaacaactgggtttggcctcccccccccccccgccccacatgatcagtaagttctatttgcaaaggccacaatcttcaggggtgagagaaaaatctgaaagacgtggagacctgtgagaaaaccagggcaaagtatctcaggccagaagtgtgctgtaacattgtgacattgtaacatcctgcagatggagaccccacccccacccccagtccccttccaccagaggccgaagcctgaaagcagacagttgctgtccttattcccagtaaaagcctctgatactctgcgaacagcacactgtggggacagtgggcagctcggaactcggctgacaccagaggtggaaccatcacttcctccagagggtggacatccgaaggatggacactttctgttaaggcacaagtgagtcagagtattttcccagagctgggctggagggggcctgagctggaagtgacactgtaagactgagtcagcaccccctgggtctggatagtgggatccctcaggggacaaggacgggcatacacagagaagaccatggccttgtgaccacaggattcatgatttctgatactgctgatccaatatgcgctcaaataaataaagactgttagtcaaaaaaaaaaaaaaaaaaaaaaaaaaaaa

SEQ ID NO: 39 - protein, 500 aa, Homo sapiens
MPPAVGGPVGYTPPDGGWGWAVVIGAFISIGFSYAFPKSITVFFKEIEGIFHATTSEVSWISSIMLAVMYGGGPISSILVNKYGSRIVMIVGGCLSGCGLIAASFCNTVQQLYVCIGVIGGLGLAFNLNPALTMIGKYFYKRRPLANGLAMAGSPVFLCTLAPLNQVFFGIFGWRGSFLILGGLLLNCCVAGALMRPIGPKPTKAGKDKSKASLEKAGKSGVKKDLHDANTDLIGRHPKQEKRSVFQTINQFLDLTLFTHRGFLLYLSGNVIMFFGLFAPLVFLSSYGKSQHYSSEKSAFLLSILAFVDMVARPSMGLVANTKPIRPRIQYFFAASVVANGVCHMLAPLSTTYVGFCVYAGFFGFAFGWLSSVLFETLMDLVGPQRFSSAVGLVTIVECCPVLLGPPLLGRLNDMYGDYKYTYWACGVVLIISGIYLFIGMGINYRLLAKEQKANEQKKESKEEETSIDVAGKPNEVTKAAESPDQKDTEGGPKEEESPV

SEQ ID NO: 40 - DNA, 3927 bp, Homo sapiens
agagggcgcgcgcggctgaaagcgtgtggaggcgcgggctgcagttcggatgtctgtgtggcggggagggggcggcggccgggagagacgactccgccccctgcgcgcatgctccggccccggcgggttataaggcagcctcgctggcccggccagacaaagtggtgagctgcgacgtgactggctagctgcgtgggtactggaacaagcaaacgaggcagcgagcgaaggacgggagccggaccctgggccccgtggaactccagcctgcgccaccacgtcacgcacacgctcggcgctgcgatccgcgcatataacgatatttggatttgacctgcattttggaatttatctacacttaaaatgccaccagcagttggaggtccagttggatacacccccccagatggaggctggggctgggcagtggtaattggagctttcatttccatcggcttctcttatgcatttcccaaatcaattactgtcttcttcaaagagattgaaggtatattccatgccaccaccagcgaagtgtcatggatatcctccataatgttggctgtcatgtatggtggaggtcctatcagcagtatcctggtgaataaatatggaagtcgtatagtcatgattgttggtggctgcttgtcaggctgtggcttgattgcagcttctttctgtaacaccgtacagcaactatacgtctgtattggagtcattggaggtcttgggcttgccttcaacttgaatccagctctgaccatgattggcaagtatttctacaagaggcgaccattggccaacggactggccatggcaggcagccctgtgttcctctgtactctggcccccctcaatcaggttttcttcggtatctttggatggagaggaagctttctaattcttgggggcttgctactaaactgctgtgttgctggagccctcatgcgaccaatcgggcccaagccaaccaaggcagggaaagataagtctaaagcatcccttgagaaagctggaaaatctggtgtgaaaaaagatctgcatgatgcaaatacagatcttattggaagacaccctaaacaagagaaacgatcagtcttccaaacaattaatcagttcctggacttaaccctattcacccacagaggctttttgctatacctctctggaaatgtgatcatgttttttggactctttgcacctttggtgtttcttagtagttatgggaagagtcagcattattctagtgagaagtctgccttccttctttccattctggcttttgttgacatggtagcccgaccatctatgggacttgtagccaacacaaagccaataagacctcgaattcagtatttctttgcggcttccgttgttgcaaatggagtgtgtcatatgctagcacctttatccactacctatgttggattctgtgtctatgcgggattctttggatttgccttcgggtggctcagctccgtattgtttgaaacattgatggaccttgttggaccccagaggttctccagcgctgtgggattggtgaccattgtggaatgctgtcctgtcctcctggggccaccacttttaggtcggctcaatgacatgtatggagactacaaatacacatactgggcatgtggcgtcgtcctaattatttcaggtatctatctcttcattggcatgggcatcaattatcgacttttggcaaaagaacagaaagcaaacgagcagaaaaaggaaagtaaagaggaagagaccagtatagatgttgctgggaagccaaatgaagttaccaaagcagcagaatctccggaccagaaagacacagatggagggcccaaggaggaggaaagtccagtctgaatccatggggctgaagggtaaattgagcagttcatgacccaggatatctgaaaatattctactggcctgtaatctaccagtggtgctcaatgcaaatagtagacatttgtgtggaaatcataccagttgttcattgatgggatttttgtttgactccttaccaatagcctgaatttgaggagggaatgattggtagcaaaggatgggggaaagaagtaggttctgttttgttttgttttaatcttagcttttaatagtgtcataaagattataatatgtgccttaagttttagtctttagaactctagagagccttaacttcttaaaccatttttgctgaattcatctatttcgagtgttgtgttaaaaggaaaaataacaactaacttgtttgaggcaaatctaaaatttaaaattaatcttgcttcattgttacatgtaatatatttcagacattttcactggaagatttatgaacagaaatattggttgaaagttagagattttacaaaatgctgacaaaaatattttcctagcatcagtagatttctggcatatgtttctgctagctatatatttaggaaattcaaagcataaaactttggcaacatcttggctgttctagacacagtgtacttgtcaacccctctcaggtaccttttcttgggatgcttattagaagccaagtaaagtgcttaaggtttgttttcattaaattagctatttctgctcccctgttcaaagatgcattttgagtgtttatagatcactgccctttttgaaatcacctggtattatttttcttactggaaaagttagtattaaaatctacagaactacatatttgtgcctccttggtaaatacaacacatctaattaaatgtagacagatatttcaaacatcagctgaattcacttaagtttttccaaaacctcagttaaactgtgaagctattggaatttttttttcctggaatttttcccctttgattcacagtggtcccatttatatctgcttctagcttagtgctatgtgtgagatatgtgtgtgtttggtgtttttgtttttttgtttttttttttttaaggtttgcaaattaaaaagggccagaaaaatttggcaccaggcaaacgaataaagataggattgggaaagaagttgctaagtgtgcttagttttaataagtaattccttctcttttttcagagaaggccttacagaaaattgttgtgcttagaattgctggatgcatttttaccctccacacaaacctaaaaattttgtgacccctttcacttacctgaaaagtagagaaatggattcagtataaggataaggagggaaggtggaccagaatgaaaactgtaaatatttttttaacctaatatcacttaaatcgaggcagaaagatacagacattcaatgaattatattcaatgcatttaaaataccactgtaattgacagagtaaaagtatagatacaaaaccttgtgtaagaggctgacttttccaaataaacattttttaagaaaacatttcttctcccaaatgtctattttcttgaggaaaatattgctgtgtcttcattttcattaccaggtttcattttgggccttgctaaattgattgaattaaatcctccagcttttgaaccttgatatttgtgtatatgatttattttcatttgaatttctcctttcctcttctttgctgtaaggcaaggaggaggggaattttaaaaccatcttatttgaactgagagcatccagagcagttaaccttaaggaaacaatgaaaaactccctttgtatgcctgggcatcatggcagatagaggaagagtgttagaggagaaaactgctgctgagagtattggcaggcttggcctcagtttggactctgtaattttctttggacccaagtctgtaacctctgcgtacttcttctctcttaccttctataaaaatgaggattactgttggtgagggaataaggaatgtaagtaaaggaaatctgaaaaaataaaagtaaagcaagtataaaaaaaaaaaaaaaaaaaa

SEQ ID NO: 41 - protein, 478 aa, Homo sapiens [sequence omitted]

SEQ ID NO: 42 - DNA, 1907 bp, Homo sapiens
cgggcgcccaccctgcgccagagaccagataaagatcaatcttaagatgtgatactttcctgtgaaacctgaaacaaggtgatctggggaaccaaagactctgggactcttggtgccaacagagttactctgttacttgaatttccactagaggagcagaaatgccaccaatgccaagtgccccacctgtgcatccacctccagatggaggatggggttggattgtggttggagcaacttttatctccattggattttcctatgcattccccaaagctgtcaccgtattcttcaaagaaattcagcaaatattccacactacctacagtgaaatagcatggatttcatccattatgctggctgttatgtacgcaggaggtcctgtaagtagtgttttggtgaataaatacggcagccggccggtggtgatagcaggaggcttattatgctgtcttggaatggtgttggcctcctttagtagcagcgtggtacagctgtacctcactatgggattcattacaggtttaggtttagccttcaacctgcaacccgccttaaccataattggcaaatacttctataggaagcgacccatggcaaatggattggccatggcaggaaatcctgttttcttaagttcattggctcctttcaatcagtacctttttaatacttttggctggaaaggaagcttcctgattttgggaagtctacttttgaatgcctgtgtggctggttccctcatgagaccccttggacccaatcaaaccacttctaagtctaaaaataagactggcaaaacagaagatgattcaagcccaaagaaaatcaaaacgaagaaatcaacttgggaaaaagttaataagtatttagatttctccctttttaagcatagaggatttctgatatatctgtctggaaatgtcattatgttcctaggtttttttgcccccattatattcccggctccatatgctaaagaccaaggaattgatgagtactcggcagcttttctgctatctgttatggctttcgttgatatgtttgctaggccttctgtaggattaattgcaaactccaaatatattcgacctcgaattcagtacttcttcagttttgcaatcatgttcaatggagtgtgtcacctcttgtgcccactggcacaggactacacaagcctggtattatatgctgtattttttggccttggatttgggagtgttagcagtgttctctttgaaactctcatggacctcgtgggtgcaccaagattttccagtgccgtcggacttgtcacaattgtggagtgtggcccagttcttcttggccctcctcttgcaggtaaattggtggatttaactggagaatataaatacatgtacatgtcctgtggggctattgtggtagcagcaagcgtgtggctgctcattggcaatgctatcaactatagattgcttgcaaaggaaaggaaggaggaaaatgcaaggcagaagaccagagaatctgaacccttgagcaaatctaaacattcggaagatgttaacgtcaaagtttcaaatgcacagagtgtaacctcagaaagagaaactaacatttaacaagaatcacatctctgatttcagtgtttatgactttatctaggagtttgtttttcattttgtttttttaaagtattagaaaaggttttagctgaaatgaggagtcacaattaaggatggaggtgatattttcctcaatggcaattttaaattagtttttaaaaacttacttatttgggtagttaaattttgagattatgcatagaaagaatccatgctataggtttatttccatacctgactctgggtgtggtggttaaaatactaattttaaagtcttccagtgactttcggtcttggttatatgga

SEQ ID NO: 43 - protein, 438 aa, Schizosaccharomyces pombe; identical to SEQ ID NO: 3

SEQ ID NO: 44 - DNA, 1317 bp, Schizosaccharomyces pombe
atgggtgaactcaaggaaatcttgaaacagaggtatcatgagttgcttgactggaatgtcaaagcccctcatgtccctctcagtcaacgactgaagcattttacatggtcttggtttgcatgtactatggcaactggtggtgttggtttgattattggttctttcccctttcgattttatggtcttaatacaattggcaaaattgtttatattcttcaaatctttttgttttctctctttggatcatgcatgctttttcgctttattaaatatccttcaactatcaaggattcctggaaccatcatttggaaaagcttttcattgctacttgtcttctttcaatatccacgttcatcgacatgcttgccatatacgcctatcctgataccggcgagtggatggtgtgggtcattcgaatcctttattacatttacgttgcagtatcctttatatactgcgtaatggctttttttacaattttcaacaaccatgtatataccattgaaaccgcatctcctgcttggattcttcctattttccctcctatgatttgtggtgtcattgctggcgccgtcaattctacacaacccgctcatcaattaaaaaatatggttatctttggtatcctctttcaaggacttggtttttgggtttatcttttactgtttgccgtcaatgtcttacggttttttactgtaggcctggcaaaaccccaagatcgacctggtatgtttatgtttgtcggtccaccagctttctcaggtttggccttaattaatattgcgcgtggtgctatgggcagtcgcccttatatttttgttggcgccaactcatccgagtatcttggttttgtttctacctttatggctatttttatttggggtcttgctgcttggtgttactgtctcgccatggttagctttttagcgggctttttcactcgagcccctctcaagtttgcttgtggatggtttgcattcattttccccaacgtgggttttgttaattgtaccattgagataggtaaaatgatagattccaaagctttccaaatgtttggacatatcattggggtcattctttgtattcagtggatcctcctaatgtatttaatggtccgtgcgtttctcgtcaatgatctttgctatcctggcaaagacgaagatgcccatcctccaccaaaaccaaatacaggtgtccttaaccctaccttcccacctgaaaaagcacctgcatctttggaaaaagtcgatacacatgtcacatctactggtggtgaatcggatcctcctagtagtgaacatgaaagcgtttaa

SEQ ID NO: 45 - protein, 498 aa, Brassica napus
MEKLREIVREGRRVGEEDPRRIVHSFKVGVALVLVSSFYYYQPFGPFTDYFGINAMWAVMTVVVVFEFSVGATLSKGLNRGVATLVAGGLALGAHQLASLSGRTIEPILLATFVFVTAALATFVRFFPRVKATFDYGMLIFILTFSLISLSQFRDEEILDLAESRLSTVLVGGVSCILISIFVCPVWAGQDLHSLLVSNLDTLSHFLQEFGDEYFEARTYGNIKVVEKRRRNLERYKSVLNSKSDEDSLANFAKWEPPHGKFGFRHPWKQYLVVAALVRQCAHRIDALNSYINSNFQIPIDIKKKLEEPFRRMSLESGKAMKEASISLKKMTKSSSYDIHIINSQSACKALSTLLKSGILNDVEPLQMVSLLTTVSLLNDIVNITEKISESVRELASAARFRNKMKPTEPSVSLKKLDSGSTGCAMPINSRDGDHVVTILLSDDDKDDIDDDDTSNIVLDDDTINEKSEDGEIHVQTSCVREVGMMPEHSLGVRILQI

SEQ ID NO: 46 - DNA, 1497 bp, Brassica napus
atggagaaactgagagagatagtgagagaaggaaggagggtaggagaagaagatccgagaagaatagttcattctttcaaagtaggggttgctcttgttttggtctcttctttctattattaccaacctttcggtccttttacggattacttcggtatcaatgcaatgtgggctgtcatgaccgtcgttgttgtcttcgaattctccgtaggggccacacttagtaaaggactaaatcgaggagtggcaacattagtagcaggaggactagcacttggagctcatcagctagcaagtttgtctggtcgaacaatagagcccattcttctagccacctttgtgtttgtaacggctgcgttagcaacgttcgtgaggttcttcccaagggtgaaggcgacattcgattatgggatgttgatattcatattgacgttctcactgatatcgttgtcccagtttagagacgaagaaatattggatttggcggagagcagactatcaacagtgttggtgggaggagtcagttgcatacttatctctatctttgtctgtcctgtctgggctggacaagaccttcattctcttcttgtctccaacttggacacactctcccacttccttcaagagtttggggatgaatattttgaagccagaacatatgggaacatcaaagtggttgagaagcggagaaggaatcttgagaggtataagagtgttctcaactcaaaaagtgatgaagattctttggctaatttcgcaaaatgggaaccgcctcacggcaaatttggatttaggcatccatggaaacaatatcttgtcgtggctgcattagtcaggcaatgtgcccaccggattgatgctttgaattcatacatcaactcaaattttcagatcccaatcgacataaagaagaagttagaagaaccgtttagaagaatgagcttggagtcaggaaaagcaatgaaagaagcatcaatttcattaaagaaaatgacaaaatcatcttcttatgatattcatatcataaactcacagtctgcatgcaaagctctttccacattactcaaatcaggcatcttgaacgatgttgagcctcttcaaatggtttcattgctgacgacggtctcactactaaacgatattgtcaatataaccgaaaagatctcagaatctgtacgagagcttgcatccgctgcaagattcaggaacaagatgaagccgaccgaaccgagcgtgtcactcaagaagttggattctggaagcactggttgcgccatgccaattaattctcgtgatggcgatcatgttgtcacgatcttattaagtgatgatgataaagatgatattgatgatgatgatacatcaaacattgttcttgatgatgatacaatcaatgaaaaatctgaagacggtgaaatacatgtacaaacgagttgcgtacgtgaggtcgggatgatgccagaacattcattgggtgttcgtatacttcagatttga

SEQ ID NO: 47 - protein, 452 aa, Triticum secale
MDIDHGREIDGEMVSTIASCGLLLHSLLAGFARKVGGAAREDPRRVAHSLKVGLALALVSAVYFVTPLFNGLGVSAIWAVLTVVVVMEFTVGATLSKGLNRALATLVAGCIAVGAHQLAELTERCSDQGEPVMLTVLVFFVASAATFLRFIPEIKAKYDYGVTIFILTFGLVAVSSYRVEELIQLAHQRFYTIVVGVFICLCTTVFLFPVWAGEDVHKLASSNLGKLAQFIEGMETNCFGENNIAINLEGKDFLQVYKSVLNSKATEDSLCTFARWEPRHGQFRFRHPWSQYQKLGTLCRQCASSMEALASYVITTTKTQYPAAANPELSFKVRKTCHEMSTHSAKVLRGLEMAIRTMTVPYLANNTVVVAMKAAERLRSELEDNAALLQVMHMAVTATLLADLVDRVKEITECVDVLARLAHFKNPEDAKYAIVGALTRGIDDPLPDVVIL

SEQ ID NO: 48 - DNA, 3901 bp, Triticum secale
atggatattgatcacggcagagagatcgacggcgagatggtgagcaccatcgccagctgcgggctgctgctccactcgcttctcgccgggttcgcccggaaggtgggcggcgccgcgcgggaggacccgaggcgggtggcgcactcgctcaaggtcggcctggcgctcgcgctggtgtccgccgtctacttcgtcacgccgctcttcaacggcctcggggtctccgcgatatgggccgtgctcaccgtcgtcgtcgtcatggagttcaccgtcggtacgcgcgggcgctcgtaaatgatcagtcacattcataccacataccataaccataccagcatattatgcatgcgcgcagtgtcttctagtctgaaacgcacttgcattgtgttctccgatgccgaaacgtgacaggtgccacgctgagtaaaggcttgaaccgagccttggcgacgctggtggctggctgcatcgccgtcggagctcaccaattagctgaactaactgaacgctgtagtgaccagggagagcccgtaatgctcaccgtgctcgtcttcttcgtgggtacgtagtacacatccgcatggttaattagtacaggtttgctatttcacgtctatagatgtgagtactactgtactacccttccgcttcgagactctctctttttagttactactccttgcataatggtggactggtgttcaacgcagcgtcagcggcgacgttcttgcgcttcatcccggagatcaaagccaagtacgactacggcgtgaccatcttcatactaaccttcggtctggtggccgtgtcgagctaccgagtggaggagctcatccagctcgcgcaccagcggttctacaccatagttgtcggcgtcttcatctgcctctgcaccaccgtcttcctcttccccgtctgggccggtgaggacgtccacaaactcgcctccagcaaccttggcaaactggctcagttcattgaaggtaattaactatcacgtaccaggtcacaacatcaatatacgctaccacttgtttcaaaaagaaaaatcaatatacgctaccacgtgtacagatacattccaacattccatgacagagattcatgcataattatattttctccatattctggtttgactcttgttatcgtaaacttgtctcagtttacaaacgcaacttgatgtgggtgggcctaaatatctgaaatacatagtttgtgtcgtcgagaaaaaagaatagaccatgacaagtcgggagtagcaacagacgccctgagtagcatcgagccttaaaacggcaagccatgcttcaagctaagtcagtatatatggatctaagtgattgccaaacaacgatctctgtcgaagattctcattctaaaggagggtccctatagctagggcgcctctaactaaaatgttcttaggtcagagtcacacaaactacattcgaataacgagctagggttagcacgcgatacaagtaaccaaaggactctgatgatgacgcatggagttcatatccttgggaggaagatgggtatgtaacttgaaatatgtccgctgctggggactttttagtgtggggactttcactgcacacatatctcaacaagaatattattccccttgatcgtatgccattaacatgaaaatccacttggggtacatatgtcctttcttcttgtactggcatatagttatagacactgaggactagatataactatatgcaggaatggaaaccaactgctttggtgaaaataatattgccattaatttggagggaaaggatttcctccaagtgtacaagagcgtccttaattcgaaagccactgaggactctctggtaagcataccccacagactttttatggtacactgttatgtgtttttttttttgaaaaggaggataacccccggccaggacaatgcatacaaccatatacaccaactattatgtagacccttattttagagtaatctactctcttcatttttgtatataaagccactatgaaaaatacattttgcatcaatataaggtcatcaatggtaatcgaggcatatattaaagttgtttcctcgtactagtaatccatagcttaattactagcatgcatgtagtggatgataatgccacactcacctagtactccctccggtcctttttactctacatattaggtttgtctgaagtcaatctcatccaactttgaccaagtttatataaacaattattgatattcacataacaacaccaatatcatcagattcatctcgaaatatattttcatattatatttattagatattatagatgctaataatttctaatataaatttaatcaaacttagcaaagtttgattttcggcaaatccaatgtgcagagtaaaaaggaccggagggagtacttcttctctcacgaccacttaattttctctatgtgcatgcaccctataaattaatccggtaaacgagaaagtaaattagcttgtaaagcacacattaatttttcttcttgataaatgtaatatgagtttgtggctttgtatacaaaaatgaagggagtaatggctcagatattcaactcggtgaataccagcatcaccaccaaactaactagaagtgatacattgctagagtggtgcttgggtggatacaagcgagggcaacctgggctgctgccctaggccgcctaagaacaaaaacccacatatttgcacgtattgttcaacagtgacatcggccaatctagtccagccgcccttgtgcttttgtactgtttatttatatactttttactagtatctgtgagtggaggttgcgagagtataccgcagaaaaatataggccatggagagggggtggtaatcaaaggggattgggatacatggcctatattttttttagtgtctaaattgaggaaattggggaaatggtatatggatctagtgtctatatttttaaagagcgggtgcgggttagggaaattggggaaatggtatatggatctagtctctaagaagttttgtttgtatttaaaacagctttcatatttatttttatttttgtcgactcaaaatgttggtccgaatctggctggcaatcaatatttgatagctacagcaaaaagaacatagcattgcagcattagcatgtgataacctcataacaaaaattgaccaaaaatcttcatcaacaaaatttactcacttgggtaaaaccgtacaaactgcagtgcacctttgccagatgggagcctcgtcatggccagttcagatttcgacacccatggagccaataccagaagttgggaactctttgccgccaatgtgcatcttccatggaagctctagcttcatatgtcatcacaaccacaaaaacccaggtagaactctagcccctaaaaccaccccattttatgttcaaaacaatgccgagacaactgaatgaaataaccgaccaaaactgacatatttgttcatgttgatcatcagtaccctgcagcagccaacccagagttatccttcaaggttcgaaagacatgtcatgaaatgagcacacattccgccaaggtgcttagggggctcgaaatggcaattcgaacaatgactgtgccgtatctagcaaacaataccgtggttgtggccatgaaagcagcggaaaggctcagaagcgagcttgaagataatgcggctttgttgcaagtgatgcacatggcagtcaccgcaacgcttctcgcggacttggttgatcgggtgaaggaaatcacggaatgtgttgatgtcctagcaagactggcacactttaagaaccctgaggatgccaaatatgccattgttggtgccttaactcgagggattgacgatcctttgcctgacgtggttattttgtaa

SEQ ID NO: 49 - protein, 883 aa, Escherichia coli
MNEQYSALRSNVSMLGKVLGETIKDALGEHILERVETIRKLSKSSRAGNDANRQELLTTLQNLSNDELLPVARAFSQFLNLANTAEQYHSISPKGEAASNPEVIARTLRKLKNQPELSEDTIKKAVESLSLELVLTAHPTEITRRTLIHKMVEVNACLKQLDNKDIADYEHNQLMRRLRQLIAQSWHTDEIRKLRPSPVDEAKWGFAVVENSLWQGVPNYLRELNEQLEENLGYKLPVEFVPVRFTSWMGGDRDGNPNVTADITRHVLLLSRWKATDLFLKDIQVLVSELSMVEATPELLALVGEEGAAEPYRYLMKNLRSRLMATQAWLEARLKGEELPKPEGLLTQNEELWEPLYACYQSLQACGMGIIANGDLLDTLRRVKCFGVPLVRIDIRQESTRHTEALGELTRYLGIGDYESWSEADKQAFLIRELNSKRPLLPRNWQPSAETREVLDTCQVIAEAPQGSIAAYVISMAKTPSDVLAVHLLLKEAGIGFAMPVAPLFETLDDLNNANDVMTQLLNIDWYRGLIQGKQMVMIGYSDSAKDAGVMAASWAQYQAQDALIKTCEKAGIELTLFHGRGGSIGRGGAPAHAALLSQPPGSLKGGLRVTEQGEMIRFKYGLPEITVSSLSLYTGAILEANLLPPPEPKESWRRIMDELSVISCDVYRGYVRENKDFVPYFRSATPEQELGKLPLGSRPAKRRPTGGVESLRAIPWIFAWTQNRLMLPAWLGAGTALQKVVEDGKQSELEAMCRDWPFFSTRLGMLEMVFAKADLWLAEYYDQRLVDKALWPLGKELRNLQEEDIKVVLAIANDSHLMADLPWIAESIQLRNIYTDPLNVLQAELLHRSRQAEKEGQEPDPRVEQALMVTIAGIAAGMRNTG

SEQ ID NO: 50 - DNA, 2652 bp, Escherichia coli; identical to SEQ ID NO: 8

SEQ ID NO: 51 - protein, 883 aa, Escherichia coli
MNEQYSALRSNVSMLGKVLGETIKDALGEHILERVETIRKLSKSSRAGNDANRQELLTTLQNLSNDELLPVARAFSQFLNLANTAEQYHSISPKGEAASNPEVIARTLRKLKNQPELSEDTIKKAVESLSLELVLTAHPTEITRRTLIHKMVEVNACLKQLDNKDIADYEHNQLMRRLRQLIAQSWHTDEIRKLRPSPVDEAKWGFAVVENSLWQGVPNYLRELNEQLEENLGYKLPVEFVPVRFTSWMGGDRDGNPNVTADITRHVLLLSRWKATDLFLKDIQVLVSELSMVEATPELLALVGEEGAAEPYRYLMKNLRSRLMATQAWLEARLKGEELPKPEGLLTQNEELWEPLYACYQSLQACGMGIIANGDLLDTLRRVKCFGVPLVRIDIRQESTRHTEALGELTRYLGIGDYESWSEADKQAFLIRELNSKRPLLPRNWQPSAETREVLDTCQVIAEAPQGSIAAYVISMAKTPSDVLAVHLLLKEAGIGFAMPVAPLFETLDDLNNANDVMTQLLNIDWYRGLIQGKQMVMIGYSDSAKDAGVMAASWAQYQAQDALIKTCEKAGIELTLFHGRGGSIGRGGAPAHAALLSQPPGSLKGGLRVTEQGEMIRFSYGLPEITVSSLSLYTGAILEANLLPPPEPKESWRRIMDELSVISCDVYRGYVRENKDFVPYFRSATPEQELGKLPLGSRPAKRRPTGGVESLRAIPWIFAWTQNRLMLPAWLGAGTALQKVVEDGKQSELEAMCRDWPFFSTRLGMLEMVFAKADLWLAEYYDQRLVDKALWPLGKELRNLQEEDIKVVLAIANDSHLMADLPWIAESIQLRNIYTDPLNVLQAELLHRSRQAEKEGQEPDPRVEQALMVTIAGIAAGMRNTG

SEQ ID NO: 52 - skipped

SEQ ID NO: 53 - protein, 883 aa, Escherichia coli
MNEQYSALRSNVSMLGKVLGETIKDALGEHILERVETIRKLSKSSRAGNDANRQELLTTLQNLSNDELLPVARAFSQFLNLANTAEQYHSISPKGEAASNPEVIARTLRKLKNQPELSEDTIKKAVESLSLELVLTAHPTEITRRTLIHKMVEVNACLKQLDNKDIADYEHNQLMRRLRQLIAQSWHTDEIRKLRPSPVDEAKWGFAVVENSLWQGVPNYLRELNEQLEENLGYKLPVEFVPVRFTSWMGGDRDGNPNVTADITRHVLLLSRWKATDLFLKDIQVLVSELSMVEATPELLALVGEEGAAEPYRYLMKNLRSRLMATQAWLEARLKGEELPKPEGLLTQNEELWEPLYACYQSLQACGMGIIANGDLLDTLRRVKCFGVPLVRIDIRQESTRHTEALGELTRYLGIGDYESWSEADKQAFLIRELNSKRPLLPRNWQPSAETREVLDTCQVIAEAPQGSIAAYVISMAKTPSDVLAVHLLLKEAGIGFAMPVAPLFETLDDLNNANDVMTQLLNIDWYRGLIQGKQMVMIGYSDSAKDAGVMAASWAQYQAQDALIKTCEKAGIELTLFHGRGGSIGRGGAPAHAALLSQPPGSLKGGLRVTEQGEMIRFKYGLPEITVSSLSLYTGAILEANLLPPPEPKESWRRIMDELSVISCDVYRGYVRENKDFVPYFRSATPEQELGKLPLGSRPAKRRPTGGVESLRAIPWIFAWTQNRLMLPAWLGAGTALQKVVEDGKQSELEAMCRDWPFFSTRLGMLEMVFAGADLWLAEYYDQRLVDKALWPLGKELRNLQEEDIKVVLAIANDSHLMADLPWIAESIQLRNIYTDPLNVLQAELLHRSRQAEKEGQEPDPRVEQALMVTIAGIAAGMRNTG

SEQ ID NO: 54 - skipped

SEQ ID NO: 55 - protein, 879 aa, Erwinia carotovora
MNEQYSAMRSNVSMLGTLLGDTIKEALGENILDKVETIRKLSKSSRAGNEKHRQELLTTLQNLSNDELLPVARAFSQFLNLTNTAEQYHTISPHGEAASNPAQLSSAFKRLKESKDLSERDIRDAVESLSIELVLTAHPTEITRRTLIHKLVEVNTCLKQLDHNDLADYERNQVMRRLRQLIAQSWHTDEIRKIRPTPVDEAKWGFAVVENSLWEGVPAFLRELDEQLEQAFGYRLPVDAVPVRFTSWMGGDRDGNPNVTAEVTRHVLLLSRWKAADLFLRDIQVLVSELSMSECTPELLELAGGSEVQEPYRAIMKSLRSQLSCTLSYLEARLTGEERLPPKDLLITNEQLWEPLHACYQSLKTCGMGIIADGSLLDTLRRVRCFGVPLVRIDVRQESTRHTDALAEITRYLGLGDYESWSESDKQAFLIRELSSKRPLLPRYWEPSADTKEVLDTCRVIAKAPQGSIAAYVISMARTPSDVLAVQLLLKEAGCPFALPVAPLFETLDDLNNADDVMTQLLSIDWYRGFIQGKQMVMIGYSDSAKDAGVMAASWAQYRAQDALIKTCEKAGIALTLFHGRGGSIGRGGAPAHAALLSQPPGSLKGGLRVTEQGEMIRFKYGLPDVTISSLALYTGAILEANLLPPPEPKQEWHEVMDELSRVSCDMYRGYVRENPDFVPYFRAATPELELGKLPLGSRPAKRRPNGGVESLRAIPWIFAWTQNRLMLPAWLGAGAGLQKVVDDGKQEQLEEMCRNWPFFSTRIGMLEMVFAKADLWLAEYYDQRLVEEKLWPLGKQLRDQLAADINIVLAISNDDHLMADLPWIAESIALRNVYTDPLNVLQAELLHRSRQQKQPDADLELALMVTIAGVAAGMRNTG

SEQ ID NO: 56 - DNA, 2640 bp, Erwinia carotovora
atgaacgaacaatattccgcaatgcgcagtaacgtcagtatgctcggcacactactcggcgataccatcaaggaagcactgggtgaaaacatccttgataaagtcgaaacaatccgcaagttgtcaaaatcatcccgcgcagggaatgaaaaacatcgtcaggagttgctgaccacactgcaaaacctgtctaacgatgaactgttgcccgttgctcgcgcgttcagccaattccttaacctgaccaataccgctgagcaatatcatacgatttcaccgcacggtgaagcggcgagtaacccggcacagctttccagcgcctttaagcgcctgaaagaaagtaaagatctgagcgagcgagatatccgtgatgcagtggaatcgctgtcgatcgagctggtgctgaccgcccaccccaccgagatcacccgtcggacgctgatccacaagctggtagaagtgaatacctgcctcaaacagctcgatcacaacgatctggctgattatgaacgcaatcaggtcatgcgccgtctacgccagttgatcgcacagtcttggcataccgatgagatccgcaaaattcgccctaccccggtagatgaagccaaatggggcttcgccgtagtggaaaatagcctgtgggaaggcgtacccgcatttttacgtgaactggatgaacaactggaacaagctttcggctaccgtctgccggttgacgccgtccccgtacgctttacctcctggatgggcggcgaccgcgacggcaacccaaacgtcacggcagaagtcactcgccatgtgctgttactcagtcgctggaaagccgccgatctgttcctgcgtgatattcaggtgctggtttccgagctgtcgatgtccgaatgtacgccggagctgctggaattagcaggtggcagcgaggttcaggaaccgtaccgtgccataatgaagtcactgcgctcacagttgagctgcacgctgagctatctggaagcacgcctgacgggtgaagaacgtctgccacctaaagacttgctgattaccaacgagcagttgtgggaaccgctccacgcctgctaccagtcgctgaaaacctgcggcatgggcatcattgccgacggtagcctgctggatacgctgcgtcgcgtacgctgtttcggtgtaccgttggtgcgcattgatgtccgtcaggaaagcacccgccacaccgacgcgctggccgaaattacccgctatctgggtctgggcgattatgaaagctggtcagaatccgataagcaggcattcctgatccgtgaactcagctccaaacgcccgctgctgccgcgctactgggaaccgagtgcagataccaaagaagtgctggatacctgtcgggtcattgctaaagcaccacagggttctattgccgcttacgttatttcaatggcgcgtacgccttccgacgtgctggccgttcaactgctgctcaaagaggctggctgcccgtttgctctgcccgtcgcgccgctgtttgaaacgttagacgacctgaacaacgccgatgatgtcatgacgcaattgctaagcatcgactggtatcgcggctttattcagggcaaacagatggtcatgatcggctattccgattcggcaaaagacgcaggcgtgatggccgcatcctgggcacagtaccgtgcacaagacgcgctgatcaaaacctgtgagaaagcgggtatcgcactgacgctgttccacggacgcggtggttccatcggtcgcggtggcgcaccagctcacgccgcgctgttgtcacagccgccgggcagtctgaaaggcggcctgcgcgtgacggaacaaggcgagatgatccgctttaaatacggcctgccggatgtcaccatcagcagtctggcgctgtataccggtgcgattctggaagcgaacctgctgccaccaccggagccaaaacaggaatggcacgaggtgatggatgaactgtcccgcgtgtcctgcgatatgtaccgcggatacgttcgtgaaaacccagatttcgttccttacttccgtgccgctacgccggagctggagctgggtaaactgccgctggggtcacgcccggctaaacgccgtccgaacggcggcgtggaaagcctgcgcgcgatcccgtggattttcgcctggacgcagaaccgtctgatgctaccagcgtggctcggtgccggagctgggctacagaaagtggtcgatgacggcaagcaggagcagttggaagaaatgtgtcgcaactggccgttcttctcgacgcgtatcggcatgctggagatggtgttcgccaaagccgacctatggctggcagagtattacgatcagcgtttggtagaagagaaactgtggccgttaggaaaacagctgcgcgatcagttagccgccgacatcaacattgtgctggcgatctccaatgacgatcacctgatggcagatctgccgtggatcgccgagtcgatcgcactacgtaatgtctacaccgatcccctgaacgtgctgcaggccgaactgctgcatcgttcacgtcagcaaaaacaaccggatgccgacttggagctggcgctgatggtgaccatcgctggcgtcgcagcaggtatgcgtaatacgggctaa

SEQ ID NO: 57 - protein, 1011 aa, Thermosynechococcus vulcanus
MTSVLDVTNRDRLIESESLAARTLQERLRLVEEVLVDVLAAESGQELVDLLRRLGALSSPEGHVLHAPEGELLKVIESLELNEAIRAARAFNLYFQIINIVEQHYEQQYNRERAAQEGLRRRSVMSEPISGVSGEGFPLPHTAANATDVRSGPSERLEHSLYEAIPATQQYGSFAWLFPRLQMLNVPPRHIQKLLDQLDIKLVFTAHPTEIVRQTIRDKQRRVARLLEQLDVLEGASPHLTDWNAQTLRAQLMEEIRLWWRTDELHQFKPEVLDEVEYTLHYFKEVIFAVIPKLYRRLEQSLHETFPALQPPRHRFCRFGSWVGGDRDGNPYVKPEVTWQTACYQRNLVLEEYIKSVERLINLLSLSLHWCDVLPDLLDSLEQDQRQLPSIYEQYAVRYRQEPYRLKLAYVLKRLQNTRDRNRALQTYCIRRNEAEELNNGQFYRHGEEFLAELLLIQRNLKETGLACRELDDLICQVEVFGFNLAALDIRQESTCHAEALNEITAYLGILPCPYTELSEAERTRWLLSELSTRRPLIPGELPFSDRTNEIIETFRMVRQLQQEFGTDLCNTYIISMSHEVSDLLEVLLFAKEAGLFDPATGASTLQAIPLFETVEDLKHAPAVLTQLFSLPFCRSYLGSNSTPFLQEVMLGYSDSNKDSGFLSSNWEIYKAQQQLQKIAESFGFQLRIFHGRGGSVGRGGGPAYAAILAQPAQTIKGRIKITEQGEVLASKYSLPELALFNLETVATAVIQASLLRSSIDEIEPWHEIMEELATRSRQCYRHLIYEQPEFIEFFNEVTPIQEISQLQISSRPTRRGGKKTLESLRAIPWVFSWTQTRFLLPAWYGVGTALKEFLEEKPAEHLSLLRYFYYKWPFFRMVISKVEMTLAKVDLEIARYYVQELSQPQNREAFCRLYDQIAQEYRLTTELVLTITGHERLLDGDPALQRSVQLRNRTIVPLGFLQVSLLKRLRQHNSQTTSGAILRSRYGRGELLRGALLTINGIAAGMRNTG

SEQ ID NO: 58 - DNA, 3241 bp, Thermosynechococcus vulcanus
gtgaattatggcgttcgcgatcgcctgcaacacctggaactgattctgagcagcaaaacagcttagaactgttaacaaatccttaatacgcctgttatcaattgttgtaccgccctggttcaggattgggtaaagtaaggagtattatctttgcaggctggggtaaatatgacatcagtcctcgatgtgaccaatcgcgatcgcttaattgaaagtgaaagtttggcagcccgtaccctacaggaacggttgcgactggtggaagaggtcttggtcgatgtcttggcggcagaatcgggtcaagaattggttgatctattgcggcgcttgggggctctctcttcgccggaaggtcatgtgctccatgccccagaaggggaattgctgaaggttattgaatccctcgaactcaatgaggccattagagcggcccgggcttttaacctctactttcaaattatcaacatcgttgagcagcattacgaacaacaatacaaccgtgaacgcgctgcccaagagggattgcgccgccgcagtgtcatgagtgaaccaatttccggtgtcagtggtgaaggctttccgctgcctcatactgctgccaacgcaacggatgtgcgcagtgggccgagtgaacgcctagagcatagtctctacgaagccattcccgctactcagcagtatggttcctttgcttggctctttcctcggctgcagatgctgaatgtgccgccgcgccatattcaaaagcttttggatcaactggacattaagttggttttcactgctcacccgacggagattgtgcggcaaacgattcgtgataagcagcggcgggttgcccgattacttgagcaactggatgtgctggagggggcttctccacacctaacggattggaacgcccaaactttacgggcacaactgatggaggaaattcgcctctggtggcgcaccgatgagttgcaccaatttaagccggaggtgctcgatgaggtggaatacaccctccactacttcaaggaggtcatttttgctgtcattcccaagctctatcgccgtctggagcagtcattacatgaaacctttcccgcgcttcagcccccccgtcaccgtttctgccgctttggctcttgggtggggggcgatcgcgatggcaatccctatgtcaaaccagaagtaacgtggcaaacggcctgctatcagcgcaacttagttcttgaggagtatattaagtccgttgagcgcttaatcaatttgctcagcctgtccctgcactggtgcgatgtgctgccagatttgctagattcccttgagcaggatcaacggcaactcccgagtatctatgagcagtatgcggtgcgctatcggcaggaaccctaccgcctgaaactggcctatgtgctcaaacggctgcaaaatacccgcgatcgcaaccgggcgctgcaaacctattgcattcgccgcaatgaggcggaagagttaaataatggacagttttaccgccacggtgaagaattcttggcagaactgctgctcattcagcgtaacctcaaggaaacgggattggcctgccgcgaattggatgatttgatttgccaggtggaggtctttggctttaatctagcagccttggatattcgccaagaaagtacctgtcacgctgaggccctcaatgaaattaccgcctatttgggtattctcccctgtccctatacagaactctcagaagccgaacgcacccgctggctcctcagtgaactctcgacccgtcgccccttgattccaggggaactcccctttagcgatcgcaccaatgaaatcattgaaacattccgcatggtgcggcaactccagcaggaatttggcaccgatttgtgcaatacctacatcatcagcatgagccatgaggtcagcgatctgttggaggtactcctctttgctaaggaggcaggcctttttgatccagccactggcgctagtaccctgcaagccattcccctgtttgaaacggtggaggatctcaagcacgccccagcggtgctgacccaactattctctctccccttttgccggagctatcttggaagcaacagtaccccctttctgcaggaggtcatgctgggctattccgacagcaataaggattcgggcttcctcagtagcaactgggaaatttataaggcacaacaacagctgcagaaaattgctgagagttttggcttccaactgcgcattttccacggtcggggtggttcagtgggtcggggtggtggacctgcctatgcggcgattttggcacagccagcacaaacgattaagggacgaatcaagattactgagcagggggaggtactggcttccaaatactcgttgccggaactcgcgctctttaacctcgaaacagtggccacagcggtcatccaagctagtttgctccgcagtagtattgatgagattgagccttggcacgagattatggaggagttggctacgcgatcgcgccagtgctatcgccatctcatctatgagcagccagaattcattgaattctttaacgaagtcaccccaatccaagagattagccaactgcaaattagctcacggccaacacggcggggggggaagaaaacccttgagagcctgcgggcaattccttgggtctttagttggacgcaaacccgtttcctgctgccggcttggtatggcgtgggtactgccctgaaggaattccttgaggaaaaacccgctgagcatctctccctcttgcgctacttctactataagtggcctttcttccgcatggtgatctctaaggttgagatgacccttgccaaggttgatctagagattgcccgctactatgtccaagaactcagccagccccaaaaccgtgaagccttctgccgcctctacgatcagattgctcaggaatatcgcctgaccacggaattagtcctcacgattactggccatgagcggctactcgatggggatccggcgcttcagcgatcggtgcaactgcgcaatcgcaccattgttcctttgggcttcctgcaagtatctcttttgaaacggctgcgccagcacaatagccaaaccacctctggggcaattttgcgctcccgctatggtcggggtgaattgctacggggggcactcttgaccatcaatggcatagcagcggggatgcgcaatacaggctaagcaacggcgagggtgaatcatggacccgacgacccgc

SEQ ID NO: 59 - protein, 919 aa, Corynebacterium glutamicum
MTDFLRDDIRFLGQILGEVIAEQEGQEVYELVEQARLTSFDIAKGNAEMDSLVQVFDGITPAKATPIARAFSHFALLANLAEDLYDEELREQALDAGDTPPDSTLDATWLKLNEGNVGAEAVADVLRNAEVAPVLTAHPTETRRRTVFDAQKWITTHMRERHALQSAEPTARTQSKLDEIEKNIRRRITILWQTALIRVARPRIEDEIEVGLRYYKLSLLEEIPRINRDVAVELRERFGEGVPLKPVVKPGSWIGGDHDGNPYVTAETVEYSTHRAAETVLKYYARQLHSLEHELSLSDRMNKVTPQLLALADAGHNDVPSRVDEPYRRAVHGVRGRILATTAELIGEDAVEGVWFKVFTPYASPEEFLNDALTIDHSLRESKDVLIADDRLSVLISAIESFGFNLYALDLRQNSESYEDVLTELFERAQVTANYRELSEAEKLEVLLKELRSPRPLIPHGSDEYSEVTDRELGIFRTASEAVKKFGPRMVPHCIISMASSVTDVLEPMVLLKEFGLIAANGDNPRGTVDVIPLFETIEDLQAGAGILDELWKIDLYRNYLLQRDNVQEVMLGYSDSNKDGGYFSANWALYDAELQLVELCRSAGVKLRLFHGRGGTVGRGGGPSYDAILAQPRGAVQGSVRITEQGEIISAKYGNPETARRNLEALVSATLEASLLDVSELTDHQRAYDIMSEISELSLKKYASLVHEDQGFIDYFTQSTPLQEIGSLNIGSRPSSRKQTSSVEDLRAIPWVLSWSQSRVMLPGWFGVGTALEQWIGEGEQATQRIAELQTLNESWPFFTSVLDNMAQVMSKAELRLAKLYADLIPDTEVAERVYSVIREEYFLTKKMFCVITGSDDLLDDNPLLARSVQRRYPYLLPLNVIQVEMMRRYRKGDQSEQVSRNIQLTMNGLSTALRNSG

SEQ ID NO: 60 - DNA, 2760 bp, Corynebacterium glutamicum
atgactgattttttacgcgatgacatcaggttcctcggtcaaatcctcggtgaggtaattgcggaacaagaaggccaggaggtttatgaactggtcgaacaagcgcgcctgacttcttttgatatcgccaagggcaacgccgaaatggatagcctggttcaggttttcgacggcattactccagccaaggcaacaccgattgctcgcgcattttcccacttcgctctgctggctaacctggcggaagacctctacgatgaagagcttcgtgaacaggctctcgatgcaggcgacacccctccggacagcactcttgatgccacctggctgaaactcaatgagggcaatgttggcgcagaagctgtggccgatgtgctgcgcaatgctgaggtggcgccggttctgactgcgcacccaactgagactcgccgccgcactgtttttgatgcgcaaaagtggatcaccacccacatgcgtgaacgccacgctttgcagtctgcggagcctaccgctcgtacgcaaagcaagttggatgagatcgagaagaacatccgccgtcgcatcaccattttgtggcagaccgcgttgattcgtgtggcccgcccacgtatcgaggacgagatcgaagtagggctgcgctactacaagctgagccttttggaagagattccacgtatcaaccgtgatgtggctgttgagcttcgtgagcgtttcggcgagggtgttcctttgaagcccgtggtcaagccaggttcctggattggtggagaccacgacggtaacccttatgtcaccgcggaaacagttgagtattccactcaccgcgctgcggaaaccgtgctcaagtactatgcacgccagctgcattccctcgagcatgagctcagcctgtcggaccgcatgaataaggtcaccccgcagctgcttgcgctggcagatgcagggcacaacgacgtgccaagccgcgtggatgagccttatcgacgcgccgtccatggcgttcgcggacgtatcctcgcgacgacggccgagctgatcggcgaggacgccgttgagggcgtgtggttcaaggtctttactccatacgcatctccggaagaattcttaaacgatgcgttgaccattgatcattctctgcgtgaatccaaggacgttctcattgccgatgatcgtttgtctgtgctgatttctgccatcgagagctttggattcaacctttacgcactggatctgcgccaaaactccgaaagctacgaggacgtcctcaccgagcttttcgaacgcgcccaagtcaccgcaaactaccgcgagctgtctgaagcagagaagcttgaggtgctgctgaaggaactgcgcagccctcgtccgctgatcccgcacggttcagatgaatacagcgaggtcaccgaccgcgagctcggcatcttccgcaccgcgtcggaggctgttaagaaattcgggccacggatggtgcctcactgcatcatctccatggcatcatcggtcaccgatgtgctcgagccgatggtgttgctcaaggaattcggactcatcgcagccaacggcgacaacccacgcggcaccgtcgatgtcatcccactgttcgaaaccatcgaagatctccaggccggcgccggaatcctcgacgaactgtggaaaattgatctctaccgcaactacctcctgcagcgcgacaacgtccaggaagtcatgctcggttactccgattccaacaaggatggcggatatttctccgcaaactgggcgctttacgacgcggaactgcagctcgtcgaactatgccgatcagccggggtcaagcttcgcctgttccacggccgtggtggcaccgtcggccgcggtggcggaccttcctacgacgcgattcttgcccagcccaggggggctgtccaaggttccgtgcgcatcaccgagcagggcgagatcatctccgctaagtacggcaaccccgaaaccgcgcgccgaaacctcgaagccctggtctcagccacgcttgaggcatcgcttctcgacgtctccgaactcaccgatcaccaacgcgcgtacgacatcatgagtgagatctctgagctcagcttgaagaagtacgcctccttggtgcacgaggatcaaggcttcatcgattacttcacccagtccacgccgctgcaggagattggatccctcaacatcggatccaggccttcctcacgcaagcagacctcctcggtggaagatttgcgagccatcccatgggtgctcagctggtcacagtctcgtgtcatgctgccaggctggtttggtgtcggaaccgcattagagcagtggattggcgaaggggagcaggccacccaacgcattgccgagctgcaaacactcaatgagtcctggccatttttcacctcagtgttggataacatggctcaggtgatgtccaaggcagagctgcgtttggcaaagctctacgcagacctgatcccagatacggaagtagccgagcgagtctattccgtcatccgcgaggagtacttcctgaccaagaagatgttctgcgtaatcaccggctctgatgatctgcttgatgacaacccacttctcgcacgctctgtccagcgccgatacccctacctgcttccactcaacgtgatccaggtagagatgatgcgacgctaccgaaaaggcgaccaaagcgagcaagtgtcccgcaacattcagctgaccatgaacggtctttccactgcgctgcgcaactccggctag

SEQ ID NO: 61 - protein, 1178 aa, Saccharomyces cerevisiae
MSQRKFAGLRDNFNLLGEKNKILVANRGEIPIRIFRTAHELSMQTVAIYSHEDRLSTHKQKADEAYVIGEVGQYTPVGAYLAIDEIISIAQKHQVDFIHPGYGFLSENSEFADKVVKAGITWIGPPAEVIDSVGDKVSARNLAAKANVPTVPGTPGPIETVEEALDFVNEYGYPVIIKAAFGGGGRGMRVVREGDDVADAFQRATSEARTAFGNGTCFVERFLDKPKHIEVQLLADNHGNVVHLFERDCSVQRRHQKVVEVAPAKTLPREVRDAILTDAVKLAKECGYRNAGTAEFLVDNQNRHYFIEINPRIQVEHTITEEITGIDIVAAQIQIAAGASLPQLGLFQDKITTRGFAIQCRITTEDPAKNFQPDTGRIEVYRSAGGNGVRLDGGNAYAGTIISPHYDSMLVKCSCSGSTYEIVRRKMIRALIEFRIRGVKTNIPFLLTLLTNPVFIEGTYWTTFIDDTPQLFQMVSSQNRAQKLLHYLADVAVNGSSIKGQIGLPKLKSNPSVPHLHDAQGNVINVTKSAPPSGWRQVLLEKGPAEFARQVRQFNGTLLMDTTWRDAHQSLLATRVRTHDLATIAPTTAHALAGRFALECWGGATFDVAMRFLHEDPWERLRKLRSLVPNIPFQMLLRGANGVAYSSLPDNAIDHFVKQAKDNGVDIFRVFDALNDLEQLKVGVDAVKKAGGVVEATVCFSGDMLQPGKKYNLDYYLEIAEKIVQMGTHILGIKDMAGTMKPAAAKLLIGSLRAKYPDLPIHVHTHDSAGTAVASMTACALAGADVVDVAINSMSGLTSQPSINALLASLEGNIDTGINVEHVRELDAYWAEMRLLYSCFEADLKGPDPEVYQHEIPGGQLTNLLFQAQQLGLGEQWAETKRAYREANYLLGDIVKVTPTSKVVGDLAQFMVSNKLTSDDVRRLANSLDFPDSVMDFFEGLIGQPYGGFPEPFRSDVLRNKRRKLTCRPGLELEPFDLEKIREDLQNRFGDVDECDVASYNMYPRVYEDFQKMRETYGDLSVLPTRSFLSPLETDEEIEVVIEQGKTLIIKLQAVGDLNKKTGEREVYFDLNGEMRKIRVADRSQKVETVTKSKADMHDPLHIGAPMAGVIVEVKVHKGSLIKKGQPVAVLSAMKMEMIISSPSDGQVKEVFVSDGENVDSSDLLVLLEDQVPVETKA

SEQ ID NO: 62 - DNA, 4201 bp, Saccharomyces cerevisiae
ccgcccaatgtcgcgggttgacccgcgacattgggcgggcaatactctcatcttattggctattcaacttagacaacggccagttgcgcgcacaaatttggtcatgaccgctccaccggcatgcgtgtcgtcatcccgcatcattcgggacattttggccttttccgcagtgcgtagttgacgtggggagaaagctagtgggctgtcgcatatggttggttttaggtggcgaaagtttcgccgcgatagcaatcatgtcctcgaccttaatctcaatggaatgggtcatgctcggtaagtgagtctcgctgtatataagtatatatgtaatcgcctagttcgatagatacaaggaggtcttgagtatgtagataaacgaaaagaagataacaaaaggaaaatctcagcctctccccttcctcttagacaatgtcgcaaagaaaattcgccggcttgagagataacttcaatctcttgggtgaaaagaacaaaatattggtggctaatagaggagaaattccaatcagaatttttcgtaccgctcatgaactgtctatgcagacggtagctatatattctcatgaagatcgtctttcaacgcacaaacaaaaggctgacgaagcatacgtcataggtgaagtaggccaatatacccccgtcggcgcttatttggccattgacgaaatcatttccattgcccaaaaacaccaggtagatttcatccatccaggttatgggttcttgtctgaaaattcggaatttgccgacaaagtagtgaaggccggtatcacttggattggccctccagctgaagttattgactccgtgggtgataaggtctcagctagaaacctggcagcaaaagctaatgtgcccaccgttcctggtacaccaggtcctatagaaactgtagaggaagcacttgacttcgtcaatgaatacggctacccggtgatcattaaggccgcctttggtggtggtggtagaggtatgagagtcgttagagaaggtgacgacgtggcagatgcctttcaacgtgctacctccgaagcccgtactgccttcggtaatggtacctgctttgtggaaagattcttggacaagccaaagcatattgaagttcaattgttggccgataaccacggaaacgtggttcatcttttcgaaagagactgttccgtgcagagaagacaccaaaaggttgtcgaagtggccccagcaaagactttaccccgtgaagtccgtgacgccattttgacagatgcagttaaattggccaaagagtgtggctacagaaatgcgggtactgctgaattcttggttgataaccaaaatagacactatttcattgaaattaatccaagaatccaagtggaacataccatcacagaagaaattaccggtatagatattgtggcggctcagatccaaattgcggcaggtgcctctctaccccagctgggcctattccaggacaaaattacgactcgtggctttgccattcagtgccgtattaccacggaagaccctgctaagaacttccaaccagataccggtagaatagaagtgtaccgttctgcaggtggtaatggtgttagactggatggtggtaacgcctatgcaggaacaataatctcacctcattacgactcaatgctggtcaaatgctcatgctccggttccacctacgaaatcgttcgtagaaaaatgattcgtgcattaatcgagttcagaattagaggtgtcaagaccaacattcccttcctattgactcttttgaccaatccagtatttattgagggtacatactggacgacttttattgacgacaccccacaactgttccaaatggtttcatcacaaaacagagcccaaaaacttttacattacctcgccgacgtggcagtcaatggttcatctatcaagggtcaaattggcttgccaaaattaaaatcaaatccaagtgtcccccatttgcacgatgctcagggcaatgtcatcaacgttacaaagtctgcaccaccatccggatggaggcaagtgctactagaaaaggggccagctgaatttgccagacaagttagacagttcaatggtactttattgatggacaccacctggagagacgctcatcaatctctacttgcaacaagagtcagaacccacgatttggctacaatcgctccaacaaccgcacatgcccttgcaggtcgtttcgccttagaatgttggggtggtgccacattcgatgttgcaatgagatttttgcatgaggatccatgggaacgtttgagaaaattaagatctctggtgcctaatattccattccaaatgttattgcgtggtgccaatggtgtggcttattcttcattgcctgacaatgctattgaccatttcgtcaagcaagccaaggataatggtgttgatatatttagagtctttgatgccttaaatgacttggaacaattgaaggtcggtgtagatgctgtgaagaaggcaggtggtgttgtagaagccactgtttgtttctctggggatatgcttcagccaggcaagaaatacaatttggattactacttggaaattgctgaaaaaattgtccaaatgggcactcatatcctgggtatcaaagatatggcaggtaccatgaagccagcagctgccaaactactgattggatctttgagggctaagtaccctgatctcccaatacatgttcacactcacgattctgcaggtactgctgttgcatcaatgactgcgtgtgctctggcgggcgccgatgtcgttgatgttgccatcaactcaatgtctggtttaacttcacaaccatcaatcaatgctctgttggcttcattagaaggtaatattgacactggtattaacgttgagcatgtccgtgaactagatgcatattgggcagagatgagattgttatactcttgtttcgaggctgacttgaagggcccagatccagaagtttatcaacatgaaatcccaggtggtcaattgacaaacttgttgtttcaagcccaacaattgggtcttggagaacaatgggccgaaacaaaaagagcttacagagaagccaattatttattgggtgatattgtcaaagttaccccaacttcgaaggtcgttggtgatctggcacaatttatggtctccaataaattaacttccgatgatgtgagacgcctggctaattctttggatttccctgactctgttatggatttcttcgaaggcttaatcggccaaccatatggtgggttcccagaaccatttagatcagacgttttaaggaacaagagaagaaagttgacttgtcgtccaggcctggaactagagccatttgatctcgaaaaaattagagaagacttgcagaatagatttggtgatgttgatgagtgcgacgttgcttcttataacatgtacccaagagtttatgaagacttccaaaagatgagagaaacgtatggtgatttatctgtattgccaacaagaagctttttgtctccactagagactgacgaagaaattgaagttgtaatcgaacaaggtaaaacgctaattatcaagctacaggctgtgggtgatttgaacaaaaagaccggtgaaagagaagtttactttgatttgaatggtgaaatgagaaaaattcgtgttgctgacagatcacaaaaagtggaaactgttactaaatccaaagcagacatgcatgatccattacacattggtgcaccaatggcaggtgtcattgttgaagttaaagttcataaaggatcactaataaagaagggccaacctgtagccgtattaagcgccatgaaaatggaaatgattatatcttctccatccgatggacaagttaaagaagtgtttgtctctgatggtgaaaatgtggactcttctgatttattagttctattagaagaccaagttcctgttgaaactaaggcatgaaccggttagttctcatttataatgtataatatacccgaatcttatttatttacctttcctattttttgacgaccagtaaatactaatacataattaggaacaaaagttaaataaaaaaaaaaataataatttaacgcatccaattaacgtgtccttttttcatcattaatttatctactatttcgatttaaattccatatacaataaatcctagatacattcccgaaagcatctt

SEQ ID NO: 63 - protein, 1180 aa, Saccharomyces cerevisiae; identical to SEQ ID NO: 1

SEQ ID NO: 64 - DNA, 4112 bp, Saccharomyces cerevisiae
taaatacccgggcgctggaatggtctaagaagaaaacctacagtcgatcaaaaatcccgtacgatattaaatagagtagttaacgtacttgagcgttataaactcacattttcactcaagaagaaaatggagaatggagacatggtaagtgaaagcattaaggaagaaacacagttagcacgtatttcatcgaaagtgtttgattactttctatttggcatttaaatatacatgggaacataactagataaaatatacgccatatatagtacagctagtattttcagatgtcataaaatataattaacgagtaaaaattactttttttgggatgggggtagggtttcttcttctaggacaaccaacaaatctgatgcgtcaacactttcaccatccttaatgaaaacgtctttaacttgaccatctgctggtgaagagacaaccatttccattttcatggcactcaaaacagcaatcgattcgccctttttcaccaaagaccctttatgtactttaacttctatgataacaccagccattggtgcaccgatttggtgagtatcgtggacatcagcctttggtttagcaacagattgtatgttttgtgacttgtctgcaactctgatctttcttaattcaccgttcaattcaaaatacacttctctttgcccagttttcttatttaagtcaccaacagcttgcaatttgataatcaaagtcttaccttgttcgatggtgacttcgatttcttcatcaggttctgctggtgctaggaaatttttggttggtagaactgataaatcaccgtatgtttctctgatcttttggaaatcttcatagacccttggatacatattgtaagaagcaacatcgcattcatcaatatcaccgaatctgttctgcaagtcttctctaattttttcgagatcaaatggttctaattctaaacctggacggcacgtcaactttcttctcttgtttctcaatacatcagatcttaatggttctgggaacccaccgtatggttgaccaattaaaccttcaaaaaagtccataacagagtcaggaaagtccaaagaattagctaaacgtctaatatcgtcggaagtcagtttgttagaaaccatgaattgagctaaatcaccgacaaccttagaagttggggtaactttaacaatatctcccagtaggtaattggcttctctgtaagctcttttagtttcagcccattgttcaccaagacccagttgttgagcttggaataacaagttagtcaattgaccacctgggatttcatgttggtaaacttctggatctggtcccttcaagtcggcctcgaaacaagaatacaacagtctcatttcggcccagtatgcatctaattcacgaacatgctcaacgttaatcccagtatcaatgttaccttctaatgaagccaacagtgcattaattgatggttgggaagttaagcccgacattgaattgatagctacatcgacaacatcagcacctgctagggcacatgcagtcatagacgcaacagcagtacctgcggagtcatgactgtgaacatgaattggtaaatccggatatctggttcttagggagccaattaataatttggcagcggccggtttcatagtacctgccatatccttaatacccaagatatgtgtacccatttgaactattttttcaacaacttctaggtagtagtctaagttgtatttcttacctggctgaagcatgtcaccagagtaacaaacagtagcttcgacaacaccaccggccttcttgacagcattcacaccaacttttaattgttctaaatcattcaaggcatcaaaaactctaaatatatcaacaccattatccttggcttgcttgacaaaatggtcaatagcattgtcaggtaatgaagagtaagccacaccgttggcaccacgtaataacatttggaatggaatattaggcaccagagatcttaattttctcagacgttcccatggatcctcatgcaagaatctcattgcaacgtcgaatgtagcaccaccccaacattctaaagcgaaagcacctgcaagggcatgtgcggttgttggagcgattgtagccaaatcgtgggttctgactcttgttgcaagtagagattgatgagcgtctctccaggtggtgtccatcagtagagtaccattgaactgtctgacttgcttggcaaattcagatggtcccttttccagtagcacttgtctccatccggatggtggtgcagactttgtaacgttgatgacattgccctgagcatcgtgcaaatgggggacacttggatttgattttagttttggcaagccaatttgacccttaatagaagaaccgttaactgccaagtctgccaaatagtgtaacagtttttgcgctctgttttgtgacgataccatttggaacagttgtggggtgtcgtcaataaaagtcgtccagtatgtaccctcaataaaaactggattggtcaaaagagtcaataggaagggaatgttggtcttaacacctctgattctgaattcgatcagggcacgaatcatcttcctacggacgatttcataagtagaaccagagcatgaacatttgaccagcattgagtcgtagtgaggcgagatagtagcacctgcataagcgttaccaccgtccaatctcacaccattaccaccggcagaacgatagacctccaggcgaccggtatccggttggaaattcttagagggatcttcagtggtaatacgacattggatggaaaacccacgggtggtgattttatcctgtaatagacctagttgagtcaaagtggcacctgcggcaatctggatttgggcagaaacaatgtcaataccggtgatttcttcagtgatggtatgctccacttgaattcttggattaatttcaatgaaatagtgtctgttttggttgtcaaccaagaattcggcggtacctgcgtttctgtaaccacataccttagctaatttaacagcatctgtcaaaatagcgtcacgaacttcacggggcaaagtctttgctggagcgacttcgacaactttttggtgtcttctttgcacagaacagtctctttcgaaaagatgaaccacgtttccgtggttatcagccaacaattgaacttcaatatgctttggcttgtccaagaatctttccacaaagcaggtaccattaccgaaggcagtacgggcttcggaggtagcacgttgaaaggcatctgccacgtcgtcaccttctctaacgactctcatacctctaccaccaccaccaaaggcggccttaatgatcaccgggtagccgtattcattaacgaagtcaagtgcctcttgcacagtttcgataggtcctggagtaccgggaacggtaggaacgttagctcttgctgccaagtgtctggcagagactttgtcacccacagagtcaataacttcagctggagggccgatccaagtgataccggccttcactactttgtcggcaaattccgaattttcagacaagaacccataacctggatggatgaaatccaccttatgcttctttgcaatttcgatgatctcgtccattgccaagtaagcacccacaggtgtatactggccctcctccccgataacatacgcttcgtccgccttcaacctgtgcattgaaagacggtcctcatgggagtatatggcgatggttctcatagacagctcatgagcagatctaaaaattctaatcggaatttcacctctattggcgaccaagatcttattcttttcgccgagcaaactgaaattgtccctaagaccggccaatttcttgctactgctcatattttattttgttctctatgtcccttattttctctcctagtaactgtcctttattttgcaatatagtaattggcaaaaaaatcaaactcaaatgacgggccacagatagtaagctacaagagtttggaagaaaagaggggcttgtttatgtatgtaatcaagtaaggataccgaaagtaagagaaaagaaaaaaggactaaaaatcacagaaacacaataggaaatatccgatggggagtacttcttactta

SEQ ID NO: 65 - protein, 1185 aa, Saccharomyces cerevisiae
MSSSKKLAGLRDNFCLLGEKNKILVANRGEIPIRIFRSAHELSMRTIAIYSHEDRLSMHRLKADEAYVIGEEGQYTPVGAYLAMDEIIEIAKKHKVDFIHPGYGFLSENSEFADKVVKAGITWIGPPAEVIESVGDKVSARHLAARANVPTVPGTPGPIETVQEALDFVNEYGYPVIIKAAFGGGGRGMRVVREGDDVADAFQRATSEARTAFGNGTCFVERFLDKPKHIEVQLLADKHGNVVHLFERDCSVQRRHQKVVEVAPAKTFPREVRDAILTDAVKLAKVCGYRNAGTAEFLVDNQNRHYFIEINPRIQVEHTITEEITGIDIVSAQIQIAAGATLTQLGLLQDKITTRGFSIQCRITTEDPSKNFQPDTGRLEVYRSAGGNGVRLDGGNAYAGATISPHYDSMLVKCSCSGSTYEIVRRKMIRALIEFRIRGVKTNIPFLLTLLTNPVFIEGTYWTTFIDDTPQLFQMVSSQNRAQKLLHYLADLAVNGSSIKGQIGLPKLKSNPSVPHLHDAQGNVINVTKSAPPSGWRQVLLEKGPCEFAKQVRQFNGTLLMDTTWRDAHQSLLATRVRTHDLATIAPTTAHALAGAFALECWGGATFDVAMRFLHEDPWERLRKLRSLVPNIPFQMLLRGATGVAYSSLPDNAIDHFVKQAKDNGVDIFRVFDALNDLEQLKVGVNAVKKAGGVVEATVCYSGDMLQPGKKYNLDYYLEVVEKIVQMGTHILGIKDMAGTMKPAAAKLLIGSLRTRYPDLPIHVHSHDSASTRVASMTACALAGADVVDVAINSMSGLTSQPSINALLASLEGNIDTGINVEHVRELDAYRAEMRLLYPCFEADLKGPDPEVYQHEIPGGQLTNLLFQAQQLGLGEQWAETKRAYREANYLLGDIVKVTPTSKVVGDLAQFMVSNKLTSDDIRRLANSLDFPDSVMDFFEGLIGQPYGGFPEPLRSDVLRNKRRKLTCRPGLELEPFDLEKIREDLQNRFGDIDECDVASNNMYPRVYEDFQKIRETYGDLSVLPTKNFLAPAEPDEEIEVTIEQGKTLIIKLQAVGDLNKKTGQREVYFELNGELRKIRVADKSQNIQSVAKPKADVHDTHQIGAPMAGVIIEVKVHKGSLVKKGESIAVLSAMKMEMVVSSPADGQVKDVFIRDGESVDASDLLVVLEEETLPPSPKKVIFTR

SEQ ID NO: 66 - DNA, 5704 bp, Saccharomyces cerevisiae
gaattcctctgatttcgccaataagtatttcagaaacatcccactggatcacgggttcattagtcttggtgggtacatgaagttctcgggcatgctttacattgtaataaccatatatatcatcttttgcaccaaggaaaaaccctacgtagagtatttgcccaaagtggagcccataaatacaagtgacagagggtcaaagccgataagtattgagtatgacgacggtgatgtggtgtcaactcagaatacaagcagtataaagtacatttaccgctgctttataaaagtgttgaaattgaagtccgtaagaagcctagccttcattcacatgattttcgaaatttgcctttcaatgcaacgaagccgccacaaacctgaaactactagagcaaggcttcaaaagagaagacttggctgtgacagtactcatagacctgccgttcgaaatcatatttgggtactatgttgttaaatggagctccgacaaggaccccatgattcgtgacaatagaagattaagaaacagcacgggcaccaacaaggtcatcaagttcttagttggggatgccggcgttttaacaccatggttatggggcttcttgttccgtctggcagccgcagtcttgggaagttacgtggtgaagcaatttcccaaggatggtgaaatatccacgggttatttttgtctcgtgatatccagcacctcttaggttccttcatgaataccgtccagttcattggaatatcggccttccatacaagagttgcagaccccgtgctgggtggcacatatatgacactgttaaataccctcagcaacttcggtgggacatggccgcggttaatcattatgtccatgatcaactacttcaccgtgtatagtgcactattcctggcacaaataaagtatacgtaactcacggcggcacgatgcaagcgtgcaccgagcttttgaatggcaccgtgaccatcctgcgtgacggctattacatcaccaatctcatatgtattgtagtcggacttttcctatattttggatatttgaaaaggaaaatcctccatttacaaagtctgccaatcagttcctggagatgtacgtaacgacccctcaaacaagaattgtacgacattacattcaagaaaattttaacgaggtatcgtaatatttacaaaatctttttagctactacaggaagcagaaacaaaatgagatatcaataccagtgtaaatctgttaatgagaatcatcacattatcccagttttttccatgcaagggaaaaatatactacacgacatttctatactttaattatcaattaaggtcatctttctgtacgaaaactaactacgattgtcactaacgacgtgtcccttacccgacgcagctgctgatagctaccgcacagcacagccagctcttctgtgattggcagagaggggtccttcctgcggaagacgccgcaaaggcacatatgactaacctttttttttcgcaattaattttctaatagtttttcatttttttttttacaattttggcagttacgtctcgagctatttattataagagtcagaattggcgcagggagtgttaagtaagaagtactccccatcggatatttcctattgtgtttctgtgatttttagtctttttttcttttcttctttctttcggtatccttacttgattacatacataaacaagcccctcttttcttccaaactcttgtagcttactatcgtggcccgtacatttgagtttgatttttttgccaattactatattgcaaaataaaggacagttactaggagagaaaataagagacatagagaacaaagtaaaatatgagcagtagcaagaaattggccggtcttagggacaatttctgtttgctcggcgaaaagaataagatcttggtcgccaatagaggtgaaattccgattagaatttttagatctgctcatgagctgtctatgagaactatcgccatatactcccacgaggaccgtctttcaatgcataggttgaaggcggacgaagcgtatgttatcggggaggagggccagtatacacctgtgggtgcttacttggcaatggacgagatcatcgaaattgcaaagaagcataaggtggatttcatccatccaggttatgggttcttgtctgaaaattcggaatttgccgacaaagtagtgaaggccggtatcacttggattggccctccagctgaagttattgaatctgtgggtgataaggtctctgccagacacttggcagcaagagctaacgttcctaccgttcccggtaccccaggacctatcgaaactgtgcaagaggcacttgacttcgttaatgaatacggctacccggtgatcattaaggccgcctttggtggtggtggtagaggtatgagagtcgttagagaaggtgacgacgtggcagatgcctttcaacgtgctacctccgaagcccgtactgccttcggtaatggtacctgctttgtggaaagattcttggacaagccaaagcatattgaagttcaattgttggctgataaacacggaaacgtggttcatcttttcgaaagagactgttctgtgcaaagaagacaccaaaaagttgtcgaagtcgctccagcaaagactttcccccgtgaagttcgtgacgctattttgacagatgctgttaaattagctaaggtatgtggttacagaaacgcaggtaccgccgaattcttggttgacaaccaaaacagacactatttcattgaaattaatccaagaattcaagtggagcataccatcactgaagaaatcaccggtattgacattgtttctgcccaaatccagattgccgcaggtgccactttgactcaactaggtctattacaggataaaatcaccacccgtggtttttccatccaatgtcgtattaccactgaagatccctctaagaatttccaaccggataccggtcgcctggaggtctatcgttctgccggtggtaatggtgtgagattggacggtggtaacgcttatgcaggtgctactatctcgcctcactacgactcaatgctggtcaaatgttcatgctctggttctacttatgaaatcgtccgtaggaagatgattcgtgccctgatcgaattcagaatcagaggtgtgaagaccaacattcccttcctattgactcttttgaccaatccagtttttattgagggtacttactggacgacttttattgacgacaccccacaactgttccaaatggtatcatcacaaaacagagcgcaaaaactgttacactatttggcagacttggcagttaacggttcttctattaagggtcaaattggcttgccaaaactaaaatcaaatccaagtgtcccccatttgcacgatgctcagggcaatgtcatcaacgttacaaagtctgcaccaccatccggatggagacaagtgctactggaaaagggaccatgtgaatttgccaagcaagtcagacagttcaatggtactctactgatggacaccacctggagagacgctcatcaatctctacttgcaacaagagtcagaacccacgatttggctacaatcgctccaacaaccgcacatgcccttgcaggggctttcgctttagaatgttggggtggtgcgacattcgacgttgcaatgagattcttgcatgaggatccatgggaacgtctgagaaaattaagatctctggtgcctaatattccattccaaatgttattgcgtggtgccactggtgtggcttactcttcattacctgacaatgctattgaccattttgtcaagcaagccaaggataatggtgttgatatatttagagtctttgatgccttgaatgatttagaacaattaaaagttggtgtgaatgctgtcaagaaggccggtggtgttgtcgaagctactgtttgttactctggtgacatgcttcagccaggtaagaaatacaacttagattactacctagaagttgttgaaaaaatagttcaaatgggtacacatatcttgggtattaaggatatggcaggtactatgaaaccggccgctgccaaattattaattggctccctaagaaccagatatccggatttaccaattcatgttcacagtcatgactccgcaagtactcgtgttgcgtctatgactgcatgtgccctagcaggtgctgatgttgtcgatgtagctatcaattcaatgtcgggcttaacttcccaaccatcaattaatgcactgttggcttcattagaaggtaacattgatactgggattaacgttgagcatgtccgtgaattagatgcatatagggccgaaatgagactgttgtatccttgtttcgaggccgacttgaagggcccagatccagaagtttaccaacatgaaatcccaggtggtcaattgactaacttgttattccaagctcaacaactgggtcttggtgaacaatgggctgaaactaaaagagcttacagagaagccaattacctactgggagatattgttaaagttaccccaacttctaaggttgtcggtgatttagctcaattcatggtttctaacaaactgacttccgacgatattagacgtttagctaattctttggactttcctgactctgttatggacttttttgaaggtttaattggtcaaccatacggtgggttcccagaaccattaagatctgatgtattgagaaacaagagaagaaagttgacgtgccgtccaggtttagaattagaaccatttgatctcgaaaaaattagagaagacttgcagaacagattcggtgatattgatgaatgcgatgttgcttctaacaatatgtatccaagggtctatgaagatttccaaaagatcagagaaacatacggtgatttatcagttctaccaaccaaaaatttcctagcaccagcagaacctgatgaagaaatcgaagtcaccatcgaacaaggtaagactttgattatcaaattgcaagctgttggtgacttaaataagaaaactgggcaaagagaagtgtattttgaattgaacggtgaattaagaaagatcagagttgcagacaagtcacaaaacatacaatctgttgctaaaccaaaggctgatgtccacgatactcaccaaatcggtgcaccaatggctggtgttatcatagaagttaaagtacataaagggtctttggtgaagaagggcgaatcgattgctgttttgagtgccatgaaaatggaaatggttgtctcttcaccagcagatggtcaagttaaagatgttttcattagggatggtgaaagtgttgacgcatcagatttgttggttgtcctagaagaagaaaccctacccccatccccaaaaaaagtaatttttactcgttaattatattttatgacatctgaaaatactagctgtactatatatggcgtatattttatctagttatgttccatgtatatttaaatgccaaatagaaagtaatcaaacactttcgatgaaatacgtgctaactgtgtttcttccttaatgctttcacttaccatgtctccattctccattttcttcttgagtgaaaatgtgagtttataacgctcaagtacgttaactactctatttaatatcgtacgggatttttgatcgactgtaggttttcttcttagaccattccagcgcccggg

SEQ ID NO: 67 - protein, 1191 aa, Yarrowia lipolytica
MSNVPETKVDPSLSTPEVPSQGLHSRLDKMRADSSILGSMNKILVANRGEIPIRIFRTAHELSMQTVAIYAHEDRLSMHRFKADEAYVIGDRGKYTPVQAYLQVDEIIEIAKAHGVNMVHPGYGFLSENSEFARKVEEAGMAWIGPPHNVIDSVGDKVSARNLAIKNNVPVVPGTDGPVEDPKDALKFVEKYGYPVIIKAAFGGGGRGMRVVREGDDIVDAFNRASSEAKTAFGNGTCFIERFLDKPKHIEVQLLADGQGNVVHLFERDCSVQRRHQKVVEIAPAKDLPVEVRDAILDDAVRLAEDAKYRNAGTAEFLVDEQNRHYFIEINPRIQVEHTITEEITGIDIVAAQIQIAAGATLEQLGLTQDKISTRGFAIQCRITTEDPAKQFQPDTGKIEVYRSAGGNGVRLDGGNGFAGAIISPHYDSMLVKCSCSGTTFEIARRKMIRALVEFRIRGVKTNIPFLLALLTHPTFIEGKCWTTFIDDTPSLFDLMTSQNRAQKLLAYLADLCVNGTSIKGQVGNPKLKSEVVIPVLKNSEGKIVDCSKPDPVGWRNILVEQGPEAFAKAVRKNDGVLVMDTTWRDAHQSLLATRVRTTDLLAIANETSHAMSGAFALECWGGATFDVAMRFLYEDPWDRLRKMRKAVPNIPFQMLLRGANGVAYSSLPDNAIDHFVKQAKDNGVDIFRVFDALNDLDQLKVGVDAVKKAGGVVEATVCYSGDMLNPKKKYNLEYYLDFVDRVVEMGTHILGIKDMAGTLKPAAATKLIGAIREKYPNLPIHVHTHDSAGTGVASMAAAAEAGADVVDVASNSMSGMTSQPSISALMATLEGKLSTGLDPALVRELDAYWAQMRLLYSCFEADLKGPDPEVFQHEIPGGQLTNLLFQAQQVGLGEQWKETKQAYIAANQLLGDIVKVTPTSKVVGDLAQFMVSNKLSYDDVIKQAGSLDFPGSVLDFFEGLMGQPYGGFPEPLRTEALRGQRKKLTERPGKSLPPVDFAAVRKDLEERFGHITECDIASYCMYPKVFEDYRKIVDKYGDLSIVPTRLFLEAPKTDEEFSVEIEQGKTLILALRAIGDLSMQTGLREVYFELNGEMRKISVEDKKAAVETVSRPKADPGNPNEVGAPMAGVVVEVRVHEGTEVKKGDPVAVLSAMKMEMVISAPVSGKVGEVPVKEGDSVDGSDLICKIVRA

SEQ ID NO: 68 - DNA, 4326 bp, Yarrowia lipolytica
aagttccgttcagtccggggtaaggtttcagacaaggaagtggccctaaaagcaaaagtgcggtacgaactcaatcccaccctgcccaattcatgctgcatagagttggcatgatagtggtagggtttatataaatattgctatctccccatcccacaccacggtatcgcacaccatgtccaacgttcctgagaccaaggtggatccctcgctctcgacgcccgaggtgccctcgcagggccttcacagccgcctcgacaagatgcgagccgattccagtattctgggctccatgaacaagattctgggtgagtacaaacgagtgagatagtgtgtgtgtgtgtcacctgtcgacctgttgagatgacgacacgcaccctcaattgctctcaaaagtgaagcgaaaccaacaacacgaagatgggaaaaattggcactaccacactactggtgtgggggtcacatgcaactgcgaatgtcaccatgcacgttgctcggtactctgcataaggtgacaccacgacacagaggatgcttccgccacccagagtgctcccaaagaacaaccctaacacagtcgccaaccgaggagagatccccattcgaatcttcagaaccgcccatgagctttccatgcagaccgtggctatctacgcccacgaggatcggctctctatgcaccgattcaaggccgacgaggcctatgtgattggtgaccgtggcaagtacacccccgtccaggcctaccttcaggtcgacgagatcatcgagatcgcaaaggctcacggtgtcaacatggtccaccccggatacggtttcctttccgagaactccgagtttgctcgaaaggttgaggaggccggcatggcctggattggcccccctcacaacgtcattgactctgtcggtgacaaggtctctgcccgaaacctggccattaagaacaacgttcccgttgtgcccggtactgatggccctgtcgaggaccccaaggacgctctcaagtttgtggagaagtacggttaccctgttatcatcaaggctgcctttggtggtggaggccgaggtatgcgagttgttcgagaaggagacgacattgttgatgcattcaaccgagcctcctccgaggccaagactgcatttggcaacggaacctgtttcatcgagcgattcctcgataaacccaagcacattgaggtccagcttcttgctgacggccagggcaacgttgtacatctgtttgagcgagactgttccgtccagcgacgacaccagaaggtggttgagattgctcctgccaaggacctgcccgtggaggttcgagacgccattcttgacgacgctgtccgacttgctgaggacgccaagtaccgaaacgccggtaccgccgagttcctcgttgacgagcagaaccgacactacttcatcgagatcaacccccgaattcaggttgagcacacaattaccgaagagattactggcatcgacattgttgctgcacagatccagattgccgctggtgccactcttgagcagctgggactcacccaggacaagatctccactcgaggctttgccatccaatgccgaatcacaaccgaggaccctgccaagcagttccaacccgacaccggtaagatcgaggtctaccgatctgctggaggtaacggagttcggctcgatggaggaaacggttttgccggagccatcatctcccctcactacgattccatgctggtcaagtgctcttgttccggcaccacattcgagattgctcgacgaaagatgatccgagctctggttgagttccgaatccgaggagtcaagaccaacattcccttcctgttggctcttctcacccaccccactttcatcgagggtaagtgctggaccacttttatcgacgacactccttcgctgttcgacctcatgacctcccagaaccgagcccagaagctgcttgcctaccttgccgatctctgtgtcaacggcacttccatcaagggccaggttggcaaccccaagctcaagtccgaggttgtcatccccgtgctcaagaactctgagggcaagattgtcgactgttccaagcccgaccctgttggctggagaaacattcttgtcgagcagggccccgaggcctttgccaaggccgttcgaaagaacgacggtgtgcttgtcatggacaccacttggcgtgacgcccaccagtctctgcttgccacccgagttcgaaccaccgatctgctggctatcgccaacgagacctcccacgccatgtccggtgcttttgccttggagtgctggggaggagccaccttcgatgtcgccatgcggttcctgtacgaggacccctgggaccgacttcgaaagatgcgaaaggccgtgcccaacatccccttccagatgcttctgcgaggtgccaatggtgtcgcttactcctctctacctgataacgccattgaccactttgtcaagcaggccaaggataatggagtcgacattttccgagtgtttgacgctctcaacgatctcgaccagctcaaggtcggtgttgacgctgtcaagaaggccggaggagtggttgaggccaccgtttgctactccggagacatgctcaaccccaagaagaagtacaacctcgagtactacctcgattttgtcgatcgtgtggttgagatgggcactcacattctgggtatcaaggatatggctggtactcttaagcccgccgctgctaccaagctcattggtgccattcgagagaagtaccccaacctccccatccatgtccacacacatgactctgctggtactggtgtggcctccatggctgctgccgccgaggctggagctgatgtggttgacgtggcttccaactccatgtctggtatgacctctcagccctccatctctgctctcatggctactcttgagggcaagctttccactggcctggaccctgctcttgtccgagagctcgacgcgtactgggcccagatgcgactgctgtactcctgcttcgaggctgatctcaagggtcccgatcccgaggtgttccagcacgagattcctggtggccagctgaccaaccttctcttccaggcccagcaggttggtctgggagagcagtggaaggagaccaagcaggcttacatcgctgccaaccagctgctgggcgatattgttaaggtaacccctacctcgaaggttgtcggtgatctggcccagtttatggtttccaacaagctgtcatatgatgacgtcatcaaacaggctggctctctggacttccccggttccgtgctcgacttcttcgagggtctcatgggccagccctacggtggtttccctgagcctctccgaactgaggctctgcgaggccagcgaaagaagctgaccgagcgacccggtaagtctctgccccctgttgactttgctgccgtccgaaaggacctcgaggaacgatttggccacatcactgagtgcgacattgcctcttactgcatgtaccccaaggtgtttgaggactaccgaaagattgtcgacaaatacggtgatctatccatcgtccctactcgactcttcctggaggctcccaagactgacgaggagttctctgtcgagattgagcagggtaagactctgattctcgctctgcgagccattggagacctgtccatgcagactggtctacgagaggtctactttgagctcaacggagagatgcgaaagatttctgttgaggacaagaaggctgctgtcgagactgtttctcgacctaaggccgaccctggcaaccccaacgaggtcggagctcccatggccggtgtggttgtggaggtccgagtccacgagggcaccgaggtcaagaagggcgaccccgtggctgttctgtctgccatgaagatggagatggttatctctgcccctgtttccggtaaggtgggcgaggttcctgtgaaggagggcgactctgtcgatggttccgatcttatttgcaagattgtgcgggcttaattatcaaaaatagatagttaattgatttatgattttattgaaacaccgagtttctacttgtaatgtagcttttagcgtgtatatattgtcttttttatttttttatttgctccttcattataatatgttcttgagtaagctcccatgtgtaacatcacagatacttctagtttacttgattatcttgacagcgtaacgcgccacaaattaaggttcggtatatgtaaacaccaagagccctcattgtaacacccacttcacacatctaagacactgaaatggacacggcatctctcacgaacgagc

SEQ ID NO: 69 - protein, 1192 aa, Aspergillus niger
MAAPRQPEEAVDDTEFIDDHHDQHRDSVHTRLRANSAIMQFQKILVANRGEIPIRIFRTAHELSLQTVAVYSHEDRLSMHRQKADEAYMIGKRGQYTPVGAYLAIDEIVKIALEHGVHLIHPGYGFLSENAEFARKVEQSGMVFVGPTPQTIESLGDKVSARQLAIRCDVPVVPGTPGPVERYEEVKAFTDTYGFPIIIKAAFGGGGRGMRVVRDQAELRDSFERATSEARSAFGNGTVFVERFLDRPKHIEVQLLGDNHGNVVHLFERDCSVQRRHQKVVEIAPAKDLPADVRDRILADAVKLAKSVNYRNAGTAEFLVDQQNRYYFIEINPRIQVEHTITEEITGIDIVAAQIQIAAGATLEQLGLTQDRISTRGFAIQCRITTEDPSKGFSPDTGKIEVYRSAGGNGVRLDGGNGFAGAIITPHYDSMLVKCTCRGSTYEIARRKVVRALVEFRIRGVKTNIPFLTSLLSHPVFVDGTCWTTFIDDTPELFALVGSQNRAQKLLAYLGDVAVNGSSIKGQIGEPKLKGDIIKPVLHDAAGKPLDVSVPATKGWKQILDSEGPEAFARAVRANKGCLIMDTTWRDAHQSLLATRVRTIDLLNIAHETSHALANAYSLECWGGATFDVAMRFLYEDPWDRLRKLRKAVPNIPFQMLLRGANGVAYSSLPDNAIYHFCKQAKKCGVDIFRVFDALNDVDQLEVGIKAVHAAEGVVEATICYSGDMLNPSKKYNLPYYLDLVDKVVQFKPHVLGIKDMAGVLKPQAARLLIGSIRERYPDLPIHVHTHDSAGTGVASMIACAQAGADAVDAATDSLSGMTSQPSIGAILASLEGTEHDPGLNSAQVRALDTYWAQLRLLYSPFEAGLTGPDPEVYEHEIPGGQLTNLIFQASQLGLGQQWAETKKAYESANDLLGDVVKVTPTSKVVGDLAQFMVSNKLTAEDVIARAGELDFPGSVLEFLEGLMGQPYGGFPEPLRSRALRDRRKLDKRPGLYLEPLDLAKIKSQIRENYGAATEYDVASYAMYPKVFEDYKKFVAKFGDLSVLPTRYFLAKPEIGEEFHVELEKGKVLILKLLAIGPLSEQTGQREVFYEVNGEVRQVSVDDKKASVENTARPKAELGDSSQVGAPMSGVVVEIRVHDGLEVKKGDPIAVLSAMKMEMVISAPHSGKVSSLLVKEGDSVDGQDLVCKIVKA

SEQ ID NO: 70 - DNA, 6513 bp, Aspergillus niger
agaaccggagatgtgaggcccggtgtgagatttagggaaggcaagtcttgtcccggcggtgggattttctgcacgccaggtcatgggaaccggtggaagccaggctatcgatggagtcggccatccactccggcctggtttcacttttatatgggtaatgtaatcccttccctactcgatggggaaattacctccccagacgagcggacaaggcacagttagtctacgtagccgacgtgcgtttccctcgctatctaaactactcgggacaggcgggatgcaaatagttccagcttggccgaggtagatcataggaacgttgggtgatcgtctaggagaattgtaagaattatgagcttggcccggagataccatccaccgaagcacctcgcatcgatggccattgtgcgacgatcgcagaagttgccaagctgatagatgcgttttggttgagggctgaatcggtggaaagagggcacgatgcatggctcggtcacaaagtatggacaccgggctggactggctggggctggcgtgctcgctcggatgattcttgccagcttctggatgaagatccccgtcgcctatcgccggagatacggtggggaaggcctgggttttggttcgtttactgacggctacatgatatcagggcaggagagtccgatgttgctgggtaaccttgacgagacgtctttcccgagcttggaacgctccatgagattttccaggcatgaattagcccgagcacccgaggtaagcaagagaatgcgatgcctgaaccacccgatgcattttcggtataggtgggatagtgaatgatggagaatgtgtgagagtgtgaatggtagctacactacatagcgtgcagcccgtaagaaaagccatcgtcgccgccagctattcacgcggaaaactgtccattgaccgagcgtccatggcgatgatccaactgcgaatggacgacaccccaccaaactgtaatctttctgacagccaatgaatccggacgaacggaggcactgcaaccctacccaagagaagcaaaaaaagagcagaagtctaatggaacgaggcaggtcgactggccgagcatgacattccatgggtcaccatcccacatgctgcagcccggtgtgccggtgaaggtcgagcttctgggggggcgtccactagcgccgtcatgaaattaccgactgccctccggactggagccactttactatggccatagcataccgtccagggcccagcggtgtaatctgattacagtaccgcctgccaatcgtgccgcagcgtcatcgcccgtccgggatcccctcctccgccttgcgctgacctgcccctccgctcctctgctctggcttgccgggcagccgctggagagggtcatcatccccaagtacttctatgccctcctctcctcctccttcccccctcttcttcctttcccaactttctcatctcattactctctgtccatctctctctccctcctcacgttcatccccctcttgaggtcctccccctcctcctcatcctcctttttccgtcgggtaagtcgccggatccctccaattgagtttcctaatcctcccacgatttccccgcgctttcccacgctttcccgttccgaaaccgggcctttttcttccccggaagcaccttctccccaggtgactcacgccaacgtatcactgctccgttttctggcttgctttttttcttccgagcgtcattggatcatccggctaactcgtatactatatttcccttcatccccagctcctgcgccttctctctctcttctctagatcctccttcaatagctctccatctccttgcccatactctagctccccctccccctcctcaccctcccacccttatccatccttatcaacaaacacaccccgcccgccatggctgctccccgccagcccgaggaggcggtcgatgacaccgagttcatcgatgaccaccatgaccagcaccgggactctgtccacacccgtctgcgtgccaattcggctatcatgcagttccaaaagatccttgttgccaaccgtggtgaaatccccattcgtatcttccggacggctcacgagctgtccctgcagaccgtcgccgtctactcccatgaggaccatctctccatgcaccgtcagaaggccgacgaggcctacatgattggcaagcgcggtcaatatacaccggttggggcctacttggccattgacgagatcgtcaagattgctctggagcatggtgtacacctgatccacccgggttacggtttcctgtccgagaacgccgagtttgcccgcaaggtggagcagtccggcatggttttcgtcggccctaccccccagaccatcgagagcctcggtgacaaggtctccgcccgtcagctggctatccgctgcgacgtgcccgtcgtgcccggtaccccgggccctgtcgagcgctacgaggaggtcaaggccttcaccgacacctacggcttccccatcatcatcaaggccgcctttggtggtggtggtcgtggtatgcgtgtcgttcgcgaccaggccgaactgcgtgactccttcgagcgtgccacctccgaggcccgctctgcctttggcaacggcaccgtcttcgtcgagcgcttcctcgaccgccccaagcacatcgaagtccagctgctgggtgacaaccacggcaacgtcgtccacctgttcgagcgtgactgctccgtccagcgtcgccaccagaaggtcgttgaaattgccccggccaaggacctgcctgccgatgtccgtgaccgcatcctggctgatgctgtcaagctggccaagtcggtcaactaccgcaacgccggtactgcagagttcctggttgaccagcagaaccgttactacttcattgagatcaacccccgtatccaggtcgagcacactatcaccgaagagatcacgggtatcgatatcgttgctgctcagatccagattgcggccggtgctaccctggaacagctgggtctgacccaggaccgcatctccactcgtggattcgccattcagtgccgtatcaccaccgaggacccctccaagggcttctcccccgacaccggtaagatcgaggtctaccgctccgccggtggtaacggtgtccgtctggatggtggaaacggtttcgccggtgccatcatcacccctcactacgactccatgttggtcaagtgtacctgccgtggttccacctatgagatcgctcgccgcaaggtcgttcgtgctttggtcgagttccgtatccgtggtgtcaagaccaacattcctttcctcacctctcttctgagtcaccctgtgttcgtggatggtacctgctggaccacgttcattgatgacactcccgagctgttcgcccttgtcggcagtcagaaccgtgcccagaagctgctggcttacctgggtgatgtggctgtcaacggcagcagcatcaagggccagatcggcgagcccaagctcaagggcgatatcatcaagcccgttctgcatgacgctgccggcaagcccctcgacgtctctgtccccgccaccaagggatggaagcagatcctggacagtgagggtcccgaggcttttgcccgcgctgtgcgtgccaacaagggctgcttgatcatggatactacctggcgtgatgcccaccagtcgctgctggccactcgtgtgcgtaccattgacctcctgaacatcgcccacgagacgagccacgccctcgccaacgcctacagtttggaatgctggggtggtgccaccttcgatgtcgccatgcgcttcctgtacgaggacccctgggaccgtcttcgcaagctgcgcaaggccgttcccaacatccccttccagatgttgctccgtggtgccaacggtgttgcttactcctccctccctgacaacgccatctaccacttctgtaagcaggccaagaagtgcggtgtcgacatcttccgtgtcttcgatgctctcaacgacgttgaccagctcgaggtcggtatcaaggctgtccacgctgccgagggtgttgttgaggctactatttgctacagtggtgatatgctcaaccccagcaagaagtacaacctgccttactacctcgaccttgttgataaggtggtccaattcaagccccacgtcttgggtatcaaggacatggctggtgtgctgaagccccaggctgctcgtctgctgatcggttccatccgcgagcgctaccccgacctccccatccacgtgcacacccacgattctgctggtaccggtgtggcttccatgattgcttgcgctcaggccggtgctgatgccgttgatgctgccacggacagcctttccggtatgacctcccagcccagcattggtgctatcctcgcttccctcgagggaactgagcacgaccccggcctcaactcggcccaggttcgcgcccttgacacctactgggcgcagctgcgtctcctctactcgcccttcgaggcaggtctgactggtcccgaccccgaggtttacgagcatgagatccctggtggtcaattgaccaacctgatcttccaggccagccagcttggtctgggccagcagtgggcggagacgaagaaggcttacgagtctgccaacgatcttttgggcgatgtcgtcaaggtcacccccacttccaaggtggtcggtgatttggctcagttcatggtctccaacaagttgactgctgaagatgtgattgctcgcgccggcgagcttgacttccccggttccgttctggagttcctcgagggtctcatgggccagccctacggtggattccccgagcctctgcgctctcgcgctctgcgtgaccgtcgcaagctcgacaagcgccctggtctgtacctggagccccttgacctggccaagatcaagagccagatccgggagaactatggcgcagctaccgagtacgatgtggccagctatgccatgtaccccaaggtcttcgaggattacaagaagtttgtcgccaagttcggtgatttgtccgtcctgcccacgcgttacttcttggccaagcccgagatcggcgaggaattccacgtcgagctggagaagggtaaggtgctgatcctgaagttgttggccattggtcccctctccgagcagaccggccagcgtgaggtcttctacgaagtcaacggtgaggttcgccaggttagtgtggacgacaagaaggcgtccgtcgagaacaccgcccgccccaaggccgagctgggtgacagcagccaggttggtgctcctatgagcggtgtggttgtcgagatccgcgtccacgacggcctggaagtcaagaagggtgaccccattgccgtcctgagcgccatgaagatggtatgtataaccccaaaaactatcaatcaaaactcctgaccggctgctaacatctattaggaaatggttatctctgctccccacagtggcaaggtctccagcttgctggtcaaggaaggtgactcggtggatggccaggatcttgtctgcaagatcgtcaaggcctagaagagtaacaccgttatgacggcaatgtttgaaaagctaatgtcgatgttgatgtccttgcaaccggcttatttgtcaacaaggacactatgccaatattctttttctagattagagcgtctgttatgtttatgaatattttgggttgggtttgggttatttccatatttatgccacagcttatttacgatcaatggttgccacggacatacaatgtcaaccatatgaatgacaagtgatttcaaaacattctttcagatatattcgtagtatctaggagacacagcatatatagtcatgatggatatgtacaggatcaagaccaacctccatttattcagaagtattgaatctcttctcccctagagcttcgcagcagaagcttccggctctcccacctcccgtaaagccttccagacgcgcatcgcctcatcccaaggcgcagtatcctgcacaatctcaaccgacccatcaggcttcagggtcctcatctcccgatgatgcctcgtaccccacttcacatcctccaccccatgaacaaagctaataaacggatcaataagagcccgccgaataaaacctttctcctccggctccagtacattcccattcccccaaaaccaaatcggataatcactagaatgcgtaacctcccactccggcggaacactcgcatccgcacacttagctcggtactcaatcctataccgataaagcaagtgtcccgccccgccactagcaagagcatgaatcagcccgcgctgcatatcatgcacctgcatcttggcatatacccggccgaaagcatcattgtcccaattctcgcagcacggcggcaacttgccgtccgggtagtggagcttcatgagcgtgtcaacgacgtggcgggggtagtctgcgagaagtctggaatggagagcggagagcgtgtcggcttgcggcgggaaccagg

SEQ ID NO: 71 - protein, 1128 aa, Nocardioides sp.
MFSKVLVANRGEIAIRAFRAAYELGARTVAVFPYEDRWSEHRLKADEAYEIGERGHPVRAYLDPEAIVAVAIRAGADAVYPGYGFLSENPALAEACANAGITFVGPTADVLTLTGNKARAIAAATAAGVPTLASVEPSTDVDALVESAGELPYPLFVKAVAGGGGRGMRRVDAPGQLREAVETCMREAEGAFGDPTVFIEQAVVDPRHIEVQVLADGEGHVMHLFERDCSVQRRHQKVIEIAPAPNLDPELRDRICADAVRFAKEIGYRNAGTVEFLLDAKGTYHFIEMNPRIQVEHTVTEEVTDVDLVQSQLRIASGETLADLGLSQETVTLRGAALQCRITTEDPANNFRPDTGVITTYRSPGGGGVRLDGGTVYTGAEVSAHFDSMLAKLTCRGRTFEKAVEKARRAVAEFRIRGVSTNIPFLQAVLVDPDFSSGHVTTSFIETHPQLLQARSSGDRGSRLLHYLADVTVNQPHGPAPVSIDPVTKLPEVNLDVPAPDGTRQLLLDVGPEEFARRLRAQTGVAVTDTTFRDAHQSLLATRVRTRDLLAVAGHVARTTPQLWSLEAWGGATYDVALRFLAEDPWERLAALRQAVPNICLQMLLRGRNTVGYTPYPADVTQAFVEEAAATGIDVFRIFDALNDVEQMRPAIEAVRATGTAVAEVALCYTGDLSDPDETLYTLDYYLELADRIVDAGAHVLAIKDMAGLLRVPAARTLVTALRDRFDLPVHLHTHDTPGGQLATLLAAIDAGVDAVDAATASMAGTTSQPPLSALVSATDHGPRETGLSLGAVSALEPYWEATRRVYAPFESGLPSPTGRVYRHEIPGGQLSNLRQQAIALGLGEKFEQIEDMYAAANDILGNVVKVTPSSKVVGDLALHLVAVGADPTEFADEPGKFDIPDSVIGFLNGELGDPPGGWPEPFRTKALAGRTHKPPVEELDDEQREGLAGSSPTRRRTLNELLFPGPTKEFTESRLRYGDTSVLPTLDYLYGLRRGEEHAVEIEEGKTLILGVQAITEPDERGFRTVMTTINGQLRPVSVRDRSVAAEVAAAEKADTSKPGHVAAPFQGVVSIVVEEGQQVAAGDTVATIEAMKMEASITAPVAGTVERLALSGTQAVEGGDLVLVLS

SEQ ID NO: 72 - DNA, 3387 bp, Nocardioides sp.
atgttctccaaggtcctggtcgccaaccgcggcgagatcgcgatccgggcgttccgcgccgcctacgagctcggcgcgcgcacggtggccgtcttcccgtacgaggaccgctggtccgagcaccggctcaaggccgacgaggcctacgagatcggtgagcgcggccacccggtacgcgcctacctcgaccccgaggcgatcgtcgcggtcgcgatccgcgccggcgcggacgccgtctaccccgggtacggcttcctctcggagaaccccgccctcgcggaggcgtgcgcgaacgccggcatcaccttcgtcggcccgaccgccgacgtgctcacgctgaccggcaacaaggcccgggcgatcgcggcggcgacggccgcgggggtcccgaccctcgccagcgtcgagccgtccaccgatgtcgacgcgctggtggagtccgccggggagctgccgtacccgctgttcgtgaaggccgtcgccgggggcggcggccggggcatgcggcgcgtggacgcgcccgggcagctgcgcgaggcggtcgagacctgcatgcgtgaggccgagggcgcgttcggcgacccgacggtcttcatcgagcaggccgtggtcgacccgcgccacatcgaggtgcaggtcctcgccgacggcgagggccacgtgatgcacctcttcgagcgcgactgctcggtgcagcggcgccaccagaaggtcatcgagatcgcgcccgcacccaatctcgaccccgagctgcgggaccggatctgcgcggacgccgtgcgcttcgcgaaggagatcggctaccgcaacgccggcaccgtcgagttcctgctggacgccaagggcacctaccacttcatcgagatgaacccccgcatccaggtggagcacaccgtgaccgaggaggtcaccgacgtcgacctcgtgcagtcccagctgcggatcgcgtccggcgagacgctggcggacctcgggctctcccaggagaccgtgaccctgcgcggggcggcgctccagtgccggatcaccaccgaggacccggccaacaacttccgccccgacaccggggtgatcaccacctaccgctcccccggcggcggcggcgtccgcctcgacgggggcaccgtgtacaccggcgccgaggtgtccgcccacttcgactcgatgctggccaagctcacctgtcggggccgcacgttcgagaaggcggtcgagaaggcccgccgggcggtcgccgagttccggatccggggcgtctccacgaacatcccgttcctccaggcggtgctggtcgacccggacttctcctcgggccacgtcaccacctccttcatcgagacccacccacagctgctgcaggcgcggagctcgggcgaccgcggcagcaggctgctgcactacctcgccgacgtgaccgtgaaccagccgcacggccccgcgccggtgagcatcgacccggtgaccaagctgccggaggtcaacctcgacgtcccggccccggacggcacccgccagctgctgctcgacgtcggtcccgaggagttcgcgcgccggctgcgcgcgcagaccggcgtcgcggtcaccgacaccacgttccgcgacgctcaccagagcctgctcgccacccgggtccgcacccgcgacctgctcgcggtcgccggccacgtcgcgcgcaccacaccacagctgtggtcgctcgaggcctggggcggcgcgacctacgacgtcgccctgcggttcctcgccgaggacccgtgggagcggctggccgcgctgcgccaggcggtcccgaacatctgcctccagatgctgctgcgcggccgcaacacggtcgggtacacgccgtaccccgccgacgtcacccaggccttcgtcgaggaggcggcggccaccggcatcgacgtcttccggatcttcgacgccctcaacgacgtcgagcagatgcgcccggcgatcgaggcggtgcgggcgaccggcacggccgtcgccgaggtcgccctctgctacaccggcgacctctccgacccggacgagacgctctacacgctcgactactacctcgagctcgccgaccggatcgtcgacgccggggcacatgtgctggcgatcaaggacatggccggcctgctgcgggtgcccgccgcgcgcacgctggtcaccgcgctgcgcgaccggttcgacctgcccgtgcacctgcatacccacgacaccccgggcggccagctcgccaccctgctcgcggcgatcgacgcaggcgtcgacgcggtcgacgccgcgaccgcgtcgatggcggggacgacctcgcagccgccgctctcggcgctggtctcggcaaccgaccacgggccgcgggagaccgggctctcactcggcgcggtctccgcgctcgagccgtactgggaggcgacccgccgcgtctacgcgccgttcgagtccggcctgccctcgccgaccgggcgggtctaccgccacgagatccccggcggtcagctctccaacctgcgccagcaggcgatcgcgctcggcctcggggagaagttcgagcagatcgaggacatgtacgccgccgccaacgacatcctcggcaacgtcgtcaaggtgaccccgtcctcgaaggtcgtcggcgacctggcgctgcacctggtcgcggtgggcgccgacccgaccgagttcgccgacgagcccgggaagttcgacatcccggactcggtgatcgggttcctcaacggcgagctcggcgaccccccgggcggctggcccgagccgttccgcaccaaggcgctcgccggccgcacccacaagccgccggtggaggagctcgacgacgagcagcgcgagggcctcgccgggagcagcccgacccgtcgccgtaccctcaacgagctgctgttccccggtcccaccaaggagttcaccgagtcgcggctgcggtacggcgacacctcggtgctgccgaccctggactacctctacgggctgcggcgcggcgaggagcatgcggtcgagatcgaggagggcaagaccctgatcctcggcgtgcaggcgatcaccgagcccgacgagcgtggcttccggaccgtgatgaccacgatcaacgggcagctccgcccggtcagcgtccgcgatcggagcgtggccgccgaggtggccgccgccgagaaggccgacacctcgaagcccggccacgtcgcggcgccgttccagggcgtggtgtcgatcgtggtcgaggagggccagcaggtcgccgccggcgacaccgttgccacgatcgaggcgatgaagatggaggcctcgatcaccgctccggtcgccggcacggtcgagcggctggccctgtccggcacccaggccgtcgagggcggcgacctggtgctggtgctgtcctga

SEQ ID NO: 73 - protein, 491 aa, Methanothermobacter thermautotrophicus
MFSKILVANRGEIAIRVMRACRELGIKSVAVYSEADKNALFTRYADEAYEIGKPAPSQSYLRIDRILEVAEKAGAEAIHPGYGFLAENPRLGEECEKQGIKLIGPKGSVIEAMGDKITSKKLMKKAGVPVIPGTDQGVSDPDEAARIADSIGYPVIIKASAGGGGIGMRAVYEEDELIRAMESTQSVAASAFGDPTVYIEKYLERPRHIEFQVMADESGNVIHLADRECSIQRRHQKLIEEAPSPIMTPELRERMGSAAVKAAEYIGYENAGTVEFLYSNGDFYFLEMNTRIQVEHPITEVITGVDLVKEQIRVASGEELRFTQKDINIRGHAIECRINAENPLADFAPNPGKITGYRSPGGIGVRVDSGVYMNYEIPPFYDSMISKLIVWGMDRQEAINRMKRALSEYIILGVKTTIPFHKAIMRNEAFRRGELHTHFVDEYRRGIDAEMRKIVKEDQEMVERLQSTFLPSKKVAAISAAIGTYMHSRRG

SEQ ID NO: 74 - DNA, 1476 bp, Methanothermobacter thermautotrophicus
atgttcagtaaaatccttgttgcaaaccgtggtgaaatagcaataagagttatgcgtgcctgcagggaacttggaataaagagcgtggctgtttactctgaagcagacaaaaacgcccttttcacaagatacgccgatgaggcatatgagataggaaaaccagccccatcacagagctacctcaggatcgacaggatactggaggtggccgagaaggcaggcgccgaagccatccacccgggctacggtttcctcgcagagaacccccgcctcggagaggaatgtgaaaaacagggaatcaaactcataggtcccaagggctcagttatagaggccatgggggataaaataacctcaaagaagctcatgaagaaggccggtgtacccgtgatccccggtaccgatcagggcgtcagtgaccctgatgaagccgccaggatagctgattccataggttaccccgtcattataaaggcatctgcaggcgggggcgggatcggtatgcgggctgtctacgaggaggatgaactcataagggccatggagtccacccagtcggtggctgcatcagccttcggggaccccaccgtgtacatcgagaagtaccttgaaaggccacggcacatcgaattccaggtcatggcagatgaatcaggcaacgtgatacaccttgcagacagggagtgctcgatacagaggaggcaccagaaactgatagaggaggcaccgtcccccataatgacaccggaactcagggagaggatgggatccgctgcagttaaggccgctgaatatataggctatgaaaacgcaggtacagttgagttcctttactccaacggggacttctacttccttgagatgaacaccaggatacaggtggaacatcccataacagaggttatcacaggagtcgaccttgtcaaggaacagataagggtcgcctccggagaggaacttcgcttcacccagaaggatataaacatcagggggcatgccatcgagtgccgtataaacgcagagaacccccttgcagatttcgcgccaaaccccggtaagatcacaggttacagatccccagggggtataggtgtgagggttgacagcggcgtttacatgaactatgagataccccccttctatgattccatgatatcaaaactcatagtgtggggtatggaccgccaggaggccataaaccggatgaagagggccctcagtgagtacataatactgggggttaaaaccaccatacccttccacaaggccataatgaggaacgaggcattcaggcgaggcgaactccatacacacttcgttgacgaatacagaaggggtatagacgccgagatgcgaaaaatcgttaaggaggaccaggagatggttgaacgtctccagtcaacatttctcccatcaaagaaggttgccgccatatcagcggcgataggaacctacatgcattcaaggagggggtga

SEQ ID NO: 75 - protein, 568 aa, Methanothermobacter thermautotrophicus
MKGIKVVETAFRDAHQSLLATRLRTRDMTPIAEEMDRVGFFSLEAWGGATFDTCIRYLNEDPWERLRELKEHVKRTPIQMLLRGQNLVGYKHYPDDIVRKFIEKSYENGVDVFRIFDALNDIRNMEYAIKVAREQEAHVQGVICYTISPYHTLESYVDFARELEALECDSVAIKDMAGLISPHDAYELVRALKEETDLMVNLHCHCTSGMTPMSYYAACEAGVDILDTAISPLSWGASQPPTESIVAALRDTPYDTGLDLEILKNIKKYFEEIRKKYSSILDPIAEQIDTDVLIYQIPGGMLSNLVAQLKEQNALDRYEEVLEEMPRVRKDMGYPPLVTPTSQIVGIQAVMNVLSGERYSMVTNEVKDYFRGLYGRPPAPLNEEVARKVIGDEKPIDCRPADILKPQYDECRRKGEEMGIIEKEEDILTLALYPAIAPKFLRGEIEEEPLEPPAEEMAPTGEVPTVFHVEVDGDEFEVKVVPTGYMTIEEAEPEPVDVEGAVKSTMQGMVVKLKVSEGDQVNAGDVVAVVEAMKMENDIQTPHGGVVEKIYTAEGEKVETGDIIMVIK

SEQ ID NO: 76 - DNA, 1707 bp, Methanothermobacter thermautotrophicus
atgaaagggataaaggtcgttgaaacagcattcagggatgcgcaccagtcacttcttgcaacacgcctccgtacccgtgacatgacacccatcgcagaggaaatggaccgggtgggtttcttctcactggaggcatggggcggtgcaaccttcgatacctgcatacgctacctgaatgaggatccatgggagaggctacgggaacttaaggaacacgtgaaaaggacgccgatacagatgctcctcaggggccagaaccttgtggggtacaagcactaccccgatgacatagtgaggaagttcattgagaagtcctatgaaaacggcgttgatgtcttcaggatattcgacgccctcaatgacataaggaacatggagtacgccataaaggtcgccagggaacaggaagcccatgtgcagggagtcatctgctacaccatcagcccttatcacacccttgagagttatgtggactttgcaagggaactggaggcccttgaatgtgactccgttgcaataaaggacatggcaggcctaatatctccacatgatgcctacgaactcgtgagggccctcaaggaagagaccgacctcatggtgaaccttcactgccactgcacaagtggaatgacacccatgagctactatgccgcctgtgaggccggcgttgatatacttgataccgccatatcacccctatcctggggtgcctcccagccacccacagagagcattgtggctgcactccgggacacaccctacgacacgggccttgaccttgagatcctgaagaacatcaagaagtacttcgaggagatccggaagaagtacagcagcatcctcgaccccatagccgagcagatcgacacagacgtcctcatataccagatacccgggggtatgctgtcaaaccttgttgcgcagctcaaggagcagaacgccctggaccgctatgaggaggtcctcgaggagatgccccgggtgaggaaggatatgggttaccctccccttgtaaccccaaccagtcagatagtcggtatacaggccgttatgaacgtcctgagcggtgagaggtacagcatggtcaccaatgaggtgaaggactacttccggggactctacggcagaccccctgccccattaaatgaggaggtcgcaaggaaggtcatcggagatgagaaacccatcgactgcagaccagcggacatcctcaaaccccagtatgatgagtgcaggaggaagggtgaggagatgggcataatagagaaggaggaggacatactgaccctcgccctctaccctgccattgcacctaaattcctgaggggtgaaatcgaggaggagcccctggagcccccggcagaggagatggccccgacaggtgaggtgccaaccgtcttccacgtggaggtcgatggtgacgagttcgaggtcaaggtggtccccacaggctacatgaccatcgaggaagctgaacccgaaccggtggatgttgagggtgcagttaagtccaccatgcagggcatggttgtgaagctcaaggtcagtgagggcgaccaggtgaatgccggggacgttgtcgcggttgttgaagccatgaagatggagaacgatatccagacaccccacggtggtgttgttgagaagatatacaccgctgagggtgaaaaggttgagaccggcgacataatcatggtcataaaataa

SEQ ID NO: 77 - protein, 563 aa, Saccharomyces cerevisiae
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 78 - DNA, 3023 bp, Saccharomyces cerevisiae
tatttgtatcgaggtgtctagtcttctattacactaatgcagtttcagggttttggaaaccacactgtttaaacagtgttccttaatcaaggatacctctttttttttccttggttccactaattcatcggttttttttttggaagacatcttttccaacgaaaagaatatacatatcgtttaagagaaattctccaaatttgtaaagaagcggacccagacttaagcctaaccaggccaattcaacagactgtcggcaacttcttgtctggtctttccatggtaagtgacagtgcagtaataatatgaaccaatttatttttcgttacataaaaatgcttataaaactttaactaataattagagattaaatcgcttattgcttagcgttggtagcagcagtcaacttagcttgttcaaccaagttttgtggagcatcgaagactggcaacatgatttcaatcattctgatcttagagttgtcgttgaaagacttgtcttgggtcaacttgtcccattcaccggtggtagcgactctgtgggtttcatagtccttagcaccgaaagttggcaacaaggataggtggtcccaaccttgaatttcgttgtattgagcctttggaccgtgaatcaacttttcaatggtgtaaccatcgttgttcaagacgaacaagtatggcttcaagccccatctgatcatggtggagatttcttgaacagtcaattgcaaagaaccgtcaccaatgaataagataactctcttctttggatcaatttcttcagcagcgaaagcagcacccaaggtagcaccagtggtgaaaccaatggaaccccataagacttgagagataccgtaggtgttgtttgggaaagtggtttggttgataccgaaagcggaggtaccggtttcagcaatgacaacatcaccttcttgcaagaagttacccaattggttccacatccattcttgcttcaatggggtagaagctgggacagcagcgttagctggagttctagctgggacagcaactggcttgtaacccttagcggcgtcagcaatagtggtcaacaacttttgcaaaacgaatttcatttggacacctgggaaagtggcgtttctgatcttcatgtggtcggagtggaattcgacaatgttcttggtcttgtaagagtaagagaaagaaccggtgttgaaatcagacaacaaagcaccgacagacaaaatcaagtcagcagattcaacggcttccttaacttctggcttggacaaggtaccgacgtaaacaccaccgtatcttgggtgttgttcgtcaatggaacccttacccattggggtgacgaaagctgggaattgagtcaagtcaatcaacttcttagtttcagccttgacgtcgtgtctggaacaacaagcatcagccaagataactgggttcttagcatccttgaccaaagccaagatggtgtcaatgacttccttttcggattcagcatcgtttggcttcaaagacatgtcaattggagtttgcaacaacttagctgggacgttcaagtcgaccaagttagctggcaaacctaagtagactggtctttgggtgacgtaagtggttctgatacatctgtcaatttcagctggggcggtagcaatgtcagtgatcatagcagtggtttcagaaatgttggcagacattctgtggaaaacagtgaagtcaccgttacccaaggtgtggtgcaacaacaattgcttagcttgagcagagatggatgggacaccaacaacgtgcaaaacaccgacgtgttcagcgtaagaaccggcaataccgttcaaagcagacaattcaccgacaccgaaggtggtgatgatacaagacatacccttgatacgagcgtaaccatcagcggcgtaagcagcgttcaattcgttggcgttaccagcccatctcataccttcaacttcgtagatcttgtccaacaaggacaagttgaagtcacctggcaaaccgaaaacggtgttaacgttgacttgctttaatctttcgaacaaatatttacccaaagtaatttcagacattttgattgatttgactgtgttattttgcgtgaggttatgagtagaaaataataattgagaaaggaatatgacaagaaatatgaaaataaagggaacaaacccaaatctgattgcaaggagagtgaaagagccttgtttatatatttttttttcctatgttcaacgaggacagctaggtttatgcaaaaatgtgccatcaccataagctgattcaaatgagctaaaaaaaaaatagttagaaaataaggtggtgttgaacgatagcaagtagatcaagacaccgtctaacagaaaaaggggcagcggacaatattatgcaattatgaagaaaagtactcaaagggtcggaaaaatattcaaacgatatttgcataaaatcctcaattgattgattattccatagtaaaataccgtaacaacacaaaattgttctcaaattcataaattattcattttttccacgagcctcatcacacgaaaagtcagaagagcatacataatcttttaaatgcataggttatgcattttgcaaatgccaccaggcaacaaaaatatgcgtttagcgggcggaatcgggaaggaagccggaaccaccaaaaactggaagctacgtttttaaggaaggtatgggtgcagtgtgcttatctcaagaaatattagttatgatataaggtgttgaagtttagagataggtaaataaacgcggggtgtgtttattacatgaagaagaagttagtttctgccttgcttgtttatcttgcacatcacatcagcggaacatatgctcacccagtcgcatgttgttaccctcatgaaacatgtatgagatattactttgaataggttacttgcatgcatttcggcgtgcgggtaccacctctttgcatgagcttgttcctttttcaagttgaagactatatattttattgagtttatgttatggggaggctaccct

SEQ ID NO: 79 - protein, 563 aa, Saccharomyces cerevisiae
MSEITLGKYLFERLSQVNCNTVFGLPGDFNLSLLDKLYEVKGMRWAGNANELNAAYAADGYARIKGMSCIITTFGVGELSALNGIAGSYAEHVGVLHVVGVPSISSQAKQLLLHHTLGNGDFTVFHRMSANISETTAMITDIANAPAEIDRCIRTTYTTQRPVYLGLPANLVDLNVPAKLLETPIDLSLKPNDAEAEAEVVRTVVELIKDAKNPVILADACASRHDVKAETKKLMDLTQFPVYVTPMGKGAIDEQHPRYGGVYVGTLSRPEVKKAVESADLILSIGALLSDFNTGSFSYSYKTKNIVEFHSDHIKIRNATFPGVQMKFALQKLLDAIPEVVKDYKPVAVPARVPITKSTPANTPMKQEWMWNHLGNFLREGDIVIAETGTSAFGINQTTFPTDVYAIVQVLWGSIGFTVGALLGATMAAEELDPKKRVILFIGDGSLQLTVQEISTMIRWGLKPYIFVLNNNGYTIEKLIHGPHAEYNEIQGWDHLALLPTFGARNYETHRVATTGEWEKLTQDKDFQDNSKIRMIEVMLPVFDAPQNLVKQAQLTAATNAKQ

SEQ ID NO: 80 - DNA, 3087 bp, Saccharomyces cerevisiae
tacgtatacgaattccttcaacaaaggccaaggaaataaagcaaataacaataacaccattattttaattttttttctattactgtcgctaacacctgtatggttgcaaccaggtgagaatccttctgatgcatactttatgcgtttatgcgttttgcgccccttggaaaaaaattgattctcatcgtaaatgcatactacatgcgtttatgggaaaagcctccatatccaaaggtcgcgtttcttttagaaaaactaatacgtaaacctgcattaaggtaagattatatcagaaaatgtgttgcaagaaatgcattatgcaattttttgattatgacaatctctcgaaagaaatttcatatgatgagacttgaataatgcagcggcgcttgctaaaagaacttgtatataagagctgccattctcgatcaatatactgtagtaagtcctttcctctctttcttattacacttatttcacataatcaatctcaaagagaacaacacaatacaataacaagaagaacaaaatgtctgaaataaccttaggtaaatatttatttgaaagattgagccaagtcaactgtaacaccgtcttcggtttgccaggtgactttaacttgtctcttttggataagctttatgaagtcaaaggtatgagatgggctggtaacgctaacgaattgaacgctgcctatgctgctgatggttacgctcgtatcaagggtatgtcctgtattattaccaccttcggtgttggtgaattgtctgctttgaatggtattgccggttcttacgctgaacatgtcggtgttttgcacgttgttggtgttccatccatctcttctcaagctaagcaattgttgttgcatcataccttgggtaacggtgacttcactgttttccacagaatgtctgccaacatttctgaaaccactgccatgatcactgatattgctaacgctccagctgaaattgacagatgtatcagaaccacctacactacccaaagaccagtctacttgggtttgccagctaacttggttgacttgaacgtcccagccaagttattggaaactccaattgacttgtctttgaagccaaacgacgctgaagctgaagctgaagttgttagaactgttgttgaattgatcaaggatgctaagaacccagttatcttggctgatgcttgtgcttctagacatgatgtcaaggctgaaactaagaagttgatggacttgactcaattcccagtttacgtcaccccaatgggtaagggtgctattgacgaacaacacccaagatacggtggtgtttacgttggtaccttgtctagaccagaagttaagaaggctgtagaatctgctgatttgatattgtctatcggtgctttgttgtctgatttcaataccggttctttctcttactcctacaagaccaaaaatatcgttgaattccactctgaccacatcaagatcagaaacgccaccttcccaggtgttcaaatgaaatttgccttgcaaaaattgttggatgctattccagaagtcgtcaaggactacaaacctgttgctgtcccagctagagttccaattaccaagtctactccagctaacactccaatgaagcaagaatggatgtggaaccatttgggtaacttcttgagagaaggtgatattgttattgctgaaaccggtacttccgccttcggtattaaccaaactactttcccaacagatgtatacgctatcgtccaagtcttgtggggttccattggtttcacagtcggcgctctattgggtgctactatggccgctgaagaacttgatccaaagaagagagttattttattcattggtgacggttctctacaattgactgttcaagaaatctctaccatgattagatggggtttgaagccatacatttttgtcttgaataacaacggttacaccattgaaaaattgattcacggtcctcatgccgaatataatgaaattcaaggttgggaccacttggccttattgccaacttttggtgctagaaactacgaaacccacagagttgctaccactggtgaatgggaaaagttgactcaagacaaggacttccaagacaactctaagattagaatgattgaagttatgttgccagtctttgatgctccacaaaacttggttaaacaagctcaattgactgccgctactaacgctaaacaataagctaattaacataaaactcatgattcaacgtttgtgtatttttttacttttgaaggttatagatgtttaggtaaataattggcatagatatagttttagtataataaatttctgatttggtttaaaatatcaactattttttttcacatatgttcttgtaattacttttctgtcctgtcttccaggttaaagattagcttctaatattttaggtggtttattatttaattttatgctgattaatttatttactttcgtattcggttttgtacctttagctatgatcttagctaattgaagagggtggtgtgatctttaaccataccttattatctttcagctgcttaccattttcttatattgatttttagcgaaagatttttattcacaagctttttttatccttaatgctcgaatactacaacaaaacaaaaaacattaaacagtttttaattttgtgaacaaactgaattacaaggccttacatcttatttagaatatattaagaaacagaggccaacatgccttcttaattatattgatatggacctctgtccttcctaaaaacgggtttttgttcgatgaaaaatcaccagtagagcaccatatatgaatttacaatcattgtagggaaaagaaaacttgttctgcttcgccaattgatttcatttctttttttcctttgtttttgttgtatactattaatatgcgtagttctttgtcacttagtggttttttgtttaatttataacgcgaaagctgcatactaaaaatatagagatgaatggaactccgaacaagcaagagagatattatctatctctgttccataataataaccagtagttcagttggggaacttaata

SEQ ID NO: 81 - protein, 533 aa, Saccharomyces cerevisiae
MSEITLGKYLFERLKQVNVNTIFGLPGDFNLSLLDKIYEVDGLRWAGNANELNAAYAADGYARIKGLSVLVTTFGVGELSALNGIAGSYAEHVGVLHVVGVPSISAQAKQLLLHHTLGNGDFTVFHRMSANISETTSMITDIATAPSEIDRLIRTTFITQRPSYLGLPANLVDLKVPGSLLEKPIDLSLKPNDPEAEKEVIDTVLELIQNSKNPVILSDACASRHNVKKETQKLIDLTQFPAFVTPLGKGSIDEQHPRYGGVYVGTLSKQDVKQAVESADLILSVGALLSDFNTGSFSYSYKTKNVVEFHSDYVKVKNATFLGVQMKFALQNLLKVIPDVVKGYKSVPVPTKTPANKGVPASTPLKQEWLWNELSKFLQEGDVIISETGTSAFGINQTIFPKDAYGISQVLWGSIGFTTGATLGAAFAAEEIDPNKRVILFIGDGSLQLTVQEISTMIRWGLKPYLFVLNNDGYTIEKLIHGPHAEYNEIQTWDHLALLPAFGAKKYENHKIATTGEWDALTTDSEFQKNSVI

SEQ ID NO: 82 - DNA, 5577 bp, Saccharomyces cerevisiae
gatcaccgagtttttctggaactctgaatcagtggttaaggcatcccactcacccgtagtggcgatcttgtgattttcgtactttttcgcaccaaatgcgggcaacagggcgaggtgatcccaggtctggatttcgttgtactctgcgtgaggcccatgaatcagcttttcgatagtgtagccgtcgttgttaaggacaaaaagatacggctttaacccccatctgatcatggtggagatttcttggacggttaactgcaaagacccgtcacctatgaataagatgactctcttgttggggtcaatctcctcagcggcaaaggcagcacctaaagttgctcctgttgtaaaaccgatggacccccacaacacctgcgagataccgtaggcgtccttaggaaagatagtttgattgataccgaaggcagacgtgccggtctcggaaatgataacatcaccttcttgcaagaatttggacaattcgttccacaaccactcttgtttcaagggcgtgctagcaggtacacctttgtttgcgggagttttggttggtacgggaacgctcttgtagcccttaacaacatcgggaataaccttcagtaagttttgtagtgcaaatttcatttgtacaccgaggaacgtagcgttcttcacctttacgtaatcggaatgaaactccactacatttttagtcttgtaggagtaggaaaacgaacctgtgttaaaatcagagagcaaagcaccgaccgaaaggatcaaatcagccgactcaacggcctgtttcacgtcttgtttggacagcgttcccacataaacaccgccatatctgggatgctgttcatctattgaccctttacctagaggtgtcacaaaagctgggaattgcgtcaaatcaattaacttctgggtttcttttttaacgttgtgcctagaagcacaggcatccgatagtataacagggtttttcgaattctggatcaattctagtacggtatcaataacttccttttcagcttcgggatcgttaggttttaatgatagatcaatcggtttttccaaaagagaaccaggaacctttagatctaccaaattcgctggcaaccccaagtagctaggcctttgtgttataaatgttgtcctgatcaacctatcgatttctgaaggggctgtagcaatgtctgtaatcattgatgtagtttctgagatattggcggacattctgtgaaaaacggtaaaatcaccgttacccaaggtatgatgcaacaacaattgcttagcctgagcggagatagaggggacaccaacaacatgcagtacaccgacgtgttctgcatacgatcctgcaataccattcaaggcggataattcacctacgccaaaagtagttaccagcacagataaacccttgatgcgtgcgtaaccatcggcggcataggcggcgttcagctcatttgcattaccagcccatctcaatccatctacctcgtaaatcttgtccaatagggacaagttgaagtcgcctggtagcccaaaaatggtgttaacattaacttgcttcaatctttcaaataagtattttccaagagtaatttcagacattttgttggcaatatgtttttgctatattacgtgggttttttatttatactgtatataaaagaggactgcaatagcacaagattaagatagaatggcttcaaacagccgccttttatacatattggtaaaagctcgcgaatcgcaccatatcccttatcctgtaatcaaatcgatctaggtgcagatacagatcaattcataaaaagaaattgaagcaccagtttatcactactacactatctttttcttttttttttttttttgcgaagtttcgccctttgttcaatatcacttgataagttgtgggctttttctgtcactcattcggcttaaaaagtattcgttcttttgtgttttatgaaaagggaacgtgatataaaaaaacatcctttggtgtgggacatgggcttttgtttagagaatggttatcactaccgcccccacccttgaaagccacagaaaatgaaaaagtatgtgaataaggtgtgaactctataacattttggccaaatgccacagccgatctgcatattccaatggacataatgcaacaacaattgatgtcacattctcttacacacttcgattggtccgtacgtagtactttttacataactgactcaggcgtttccttcattgaaatgctcatctattgccaagtacatagaatccacagtgcataggtttatgagatgcttggaagatgtacgatcgcctgcactatattagtatattttttcaggctttacaaaaccagaaagaaattaccgactgtaatacttaatttccatgattttaatcgtatggtccgtgaggaaagaggaattttaggtaaaaaaaaacctttgtctatcaaaacataaaagaaaagaaaaaaattaaattgaataagtcagctttttagcatgaccacagtaataatagtaatacgatatcagcatgagctgctaaacattaagaatttttgaattgtgttcgctggtaagagaaaatagggtagcattagttgcgagatggaaaattaaaatatgcgtcataaggtattttcgacgttgaagattaggggttgcaagtccgagaactaaactgccttcacgacgtcaaacttattgtatggtgacaataaaacgaattcaagaagccagagatgatgaggaaccgatgacgaggcacatggggatagaacctccgttatctccattccgcgttgagcagctctcctgcgtgctttcacagagaccctaagccaagaccgcggacggccctctttttcaaggggatcaccggtaaggggccaagcggtgaaattgcgtattgtttcctcctatcaatgatgagtacgtcgccgatccagaagggttattattgtcattgccagtactattcgtatgctgaaaaagtatagttcacttcaccctctggctgcaggctagcctagccgataatgcccccgactccctcaacaggtagaatttgtaaggattcgacgtagcctggacacattgtgcatttatcgtatcccctactgctacacgtataagaagtcgttgctttacctctattccagaactcaatcttgtcgttacttgcccttattaaaaaaatccttctcttgtctcatgccaataagatcaatcagctcagcttcacaaatgaacgtgttcggtaaaaaagaagaaaagcaagaaaaagtttactctctacaaaacggttttccgtactctcatcacccatacgcttctcaatactcaagaccagacggccctatcttactgcaagacttccatctgctggaaaatattgcaagtttcgatagagaaagagttccggagcgtgtagtccatgccaaaggtggtggttgtagactggagttcgaactaacagattctttgagtgatattacatacgccgctccataccagaatgtgggttacaaatgtcctggtcttgttcgtttttccaccgttggtggtgaaagtggtacaccagacactgcaagagacccaagaggtgtttcttttaaattctataccgagtgggggaaccatgactgggtcttcaacaatactcccgtcttcttcctcagagacgctattaagtttcccgtatttattcattcgcaaaagagagaccctcagtctcatctgaatcagtttcaggacactaccatatactgggattatctaacattgaatccggaatcaatccatcaaataacttacatgtttggtgatagaggtactcctgcttcgtgggctagtatgaacgcgtactctggtcattccttcatcatggtcaacaaagaaggtaaggacacatatgtgcaattccacgtcttgtcggatactggttttgaaaccttgactggagataaggctgctgaactgtcaggctcccaccctgattataatcaggcaaagctgttcactcaattgcaaaatggcgaaaagccaaaatttaactgttatgtgcaaacaatgacacccgaacaagcaactaagttcaggtattcggtaaatgacctaacgaaaatatggccacacaaggaattccctttgagaaaatttggtaccatcaccctaacggagaatgttgacaattatttccaagaaattgaacaagttgcattcagtccaacgaacacttgtatcccaggtattaagccttctaatgattccgttctacaagccagacttttctcctatccagacactcaacgtcatagattgggagccaactatcagcaattgcccgtcaacagaccaagaaacttgggatgtccatactccaaaggtgattcccaatacactgccgaacagtgtccatttaaagcagtgaacttccaaagggacggcccaatgagttactacaatttcggtcctgagccaaattatatttccagtttaccaaatcaaactctgaaattcaaaaatgaagacaacgacgaagtatctgataagttcaaagggatagttcttgacgaagtaacagaagtttctgtgagaaaacaggaacaagaccaaatcagaaacgagcatattgttgatgccaaaattaatcaatattactacgtttatggtattagtccactagacttcgaacagccaagagctctatatgaaaaggtatacaacgatgaacagaagaaattattcgttcataacgttgtttgccacgcttgtaagatcaaagatcctaaagtcaaaaagagagttacgcaatactttggtttgctaaacgaagatttgggtaaagtcattgcagaatgcttgggagttccttgggaacctgttgaccttgaaggttatgccaagacttggtccattgcaagtgccaattaaggcagcactatttattcataattattcgtaattatatctccataattatttattcataagttttctttaagtttattctctacatcactagttattacaaattggtcgtatgtacaagcactaatatttcatacttatcgtacttcatacatagcaaatcctgttaggaaaaatatggcagcaaaacagattggctgcctacttttgacacatttttttctaattcggtacttagatcggcatgaactcaacgattatcgcattatacggacaatctgaaaaaaaaaagtagcctaacttatgcagttgtgtgtttataacgttgaattcctaaacctcttgaaggtgtaatataacaagtaacataactaggttttcacaactcgttagtttctattaaaagaggaataaacaaattttattaggactaagaggattatacagagtaccagcagagaaaactttaaattctttctgttataataatatcttaccaaagttttttgttttctatctccacatttttcgtaccatctctttaggtaatcactctgaataacttttattcatatacaccccaatattttttaagattccacctatctccgttacataaaaggatccatgcacaattttc

SEQ ID NO: 83 - protein, 925 aa, Saccharomyces cerevisiae
MLSIQQRYNICLMAERHPKWTQLELAKWAYETFQLPKIPSQGTISRLLARKSTYMNCKEHEKDANRLRKPNNLLVRKILQEWISQSLWNGIPITSPIIQDTAQAVWHRIPAETREGNGSFSYKWISNFLSKMDVNISVLDEELPKTPKVWTFEERDVLKAYFSKIPPKDLFTLIEAFLSYNLPLDYAQYEASSIQRRIEVATVMLCSNLDGSEKLKPVVVGKYDSYKSFRNYFPNEPNDPVSQSMLGTKMAKKFDISYHSNRKAWLTSNLFHNWLVRWDKRLVAVNRKIWIVLDDSCCHRIINLRLQNIKLVYTSSNSKFLPFNWGVWDEFKTRYRIQQYQALIDLQNRISKNIQNKNKSERNECIPNGKKCLISFEQSQLTMSNAFKFIKKAWDDIPVDAIKANWKSSGLLPPEMIHLNENVSMAFKKNEVLESVLNRLCDEYYCVKKWEYEMLLDLNIENKNTNFLSTEELVESAIVEPCEPDFDTAPKGNEVHDDNFDVSVFANEDDNNQNHLSMSQASHNPDYNSNHSNNAIENTNNRGSNNNNNNNGSSNNINDNDSSVKYLQQNTVDNSTKTGNPGQPNISSMESQRNSSTTDLVVDGNYDVNFNGLLNDPYNTMKQPGPLDYNVSTLIDKPNLFLSPDLDLSTVGVDMQLPSSEYFSEVFSSAIRNNEKAASDQNKSTDELPSSTAMANSNSITTALLESRNQAQPFDVPHMNGLLSDTSKSGHSVNSSNAISQNSLNNFQHNSASVAEASSPSITPSPVAINSTGAPARSIISAPIDSNSSASSPSALEHLEGAVSGMSPSSTTILSNLQTNINIAKSLSTIMKHAESNEISLTKETINELNFNYLTLLKRIKKTRKQLNSESIKINSKNAQDHLETLLSGAAAAAATSANNLDLPTGGSNLPDSNNLHLPGNTGFF

SEQ ID NO: 84 - DNA, 4333 bp, Saccharomyces cerevisiae
gcacaaggcctgcacactattcgaagtatctgtgctattgtatggactaataatttctaattcatccttcaagcgttcttcttgcagttgctcgatgaaagtagtttcatttcttttactttctggggttttgtaccctatacctacaaaatccgggtttactacctcaatctgtgatgtctcaggtgtccactcttgctcttgcgtgaacaattctccaattacaaaatcgcttaaggaccaaggcgtgtcaagttgctcacgcatgttcattgtgattttccaatgatctatttcttccgttaaactgtagcataacttaaatattcaacttgaagctcttgatagttcaaacttgcttgcccgtagacatgaagagttaagcctatcaaatcggttatgttcaaccccatgataggatgctatcttcttaactttacatgttaagaatctcatgtcactagtataaccaccaatgaagagtctgaagtacaatcgtccaactgaaaaaaatgtattcttgtttcccaatccatttgaactggagtccaatgatgcagccaataaggtgtatttgctgtaaaggatgatttttccagaacaacatcgacaaccgcctatcataatacttttcgcaaatttgcaaagaaactctcatccggtactttaaatctccaacaaatattcgaacgtcctctgtcccactataataactgttgtatttgaacagccttggaatataataacaaacatcgccttcttgatgagctatatgtccgtacttatccatggctccaacccattattaaaagccagcttattgctcttttttcgcacagaaatggaatatcaacaagcaggatttcatatattccaatgcttacttctttttccatgcctttacatacttctttttggtgtgattcgtggttttgttgttagaatagctgctgcatatttccgttctgagtgactttgcccatcgaagtcaattacttgaaagtaagaaattatcaaaaccaatagctgcttaaaaatcagttgcaagatttgaatgaaatcagcaaagtttgatcaatcgtgactagttcgcgttttgaattttgtggtaacaccaagcagtaaagagacagctttattataaccagcatgctttccattcagcaaagatataatatttgtctaatggcggagaggcacccaaagtggacgcaacttgaattggcaaaatgggcttatgagacgttccagctgccaaaaattccatcccaaggcacaatatcgcgtttgttggcaaggaaatcaacttatatgaattgtaaagagcatgaaaaagatgcgaatagattaaggaagccaaataatcttttggttcggaaaattttacaagaatggatttctcaaagtttgtggaatggaatccctataacgtcacctattattcaagacactgcacaagctgtttggcacagaattcctgcggagactcgcgagggaaatggttcttttagttataaatggatttcgaattttttatcaaaaatggatgtcaatatttctgttttagacgaagagttacccaaaaccccaaaagtctggacatttgaagagagggatgtattgaaggcttatttctccaaaattcctccaaaggatttattcactttgatagaagcgtttctctcctacaacctaccgttggattatgctcaatatgaagcaagtagcattcaaaggcgtatagaggtggcaactgtcatgctgtgctccaatttagatggctctgaaaagttaaaacctgttgtcgtgggcaaatatgatagttacaaatcattcaggaattatttccccaatgaaccgaatgatcctgtgtcacaatcaatgttgggtactaagatggctaagaaatttgatatctcataccatagtaacaggaaagcatggctaacgagcaatcttttccacaactggttagtcaggtgggataagaggttggttgctgtgaataggaagatttggattgttttggatgattcttgctgtcatcgaataattaatttgcgccttcaaaatataaaacttgtatacacttcctcaaattcaaagtttttgccatttaactggggtgtctgggatgaattcaaaacacgatacagaatacaacagtatcaggcgctcattgacttgcaaaatagaatttcgaagaatatccaaaataaaaataaatcagaacggaacgaatgcatacccaatggtaaaaaatgtttgattagctttgagcagagtcaactcacaatgtcaaatgcattcaaatttattaaaaaagcttgggatgatatacccgttgatgctatcaaagcaaattggaaaagttccggtctgcttcctcctgaaatgatacatttgaatgagaatgttagtatggcatttaagaaaaacgaagtcttagagagcgttttgaatagattatgtgatgaatactactgtgttaaaaaatgggaatatgaaatgttgttagatttaaacattgaaaacaaaaacacaaacttcttgagtacagaagaattagtggaaagtgctattgtggagccttgtgaacctgattttgatactgcgccaaaaggtaatgaggtccatgatgataattttgatgtatcagtttttgccaatgaagatgataataatcaaaatcatttaagcatgtcacaagctagccacaaccccgattacaacagtaatcacagcaacaatgctattgaaaatactaataatagaggcagtaataataataacaataataatggtagtagtaataatattaatgataatgatagtagcgtaaagtatttgcaacagaatactgttgataatagtaccaaaacaggtaaccctggacaaccaaatatttctagtatggaatcgcaaaggaactcttcgactacagatttagttgttgacggtaattatgacgtcaattttaacggccttttgaatgatccatataatacaatgaaacagccgggcccattagattataatgtcagtacattaatcgataaacctaatttattcttaagtcctgatttggatttatctactgttggcgttgatatgcaactaccatcatcagaatattttagcgaagtattttcttcagctatcagaaacaacgaaaaagctgcctcagatcagaacaaatcaactgatgaacttccttcaagcacggccatggcaaattcaaactcgataacgactgcccttctagagtcaagaaatcaagcacagccgtttgatgtcccacatatgaatgggttgctgagcgacacatcaaaaagcggacattctgttaattcctcaaatgctatatctcaaaattctctgaataactttcaacataattcggcgtccgtcgcggaagcttcgtctccttcaattacaccatctcctgtggcaataaactcaacaggcgctccagcgagatctattatatctgcacccatagactcaaattcctctgcgtcatcgccatcagctttagaacatcttgaaggtgctgtttccggtatgtcaccctcttccaccacaatattaagtaacttacaaacaaatataaatatcgccaaatcattgagtaccattatgaaacatgcagaatcaaacgaaatatcactgacgaaagaaacaataaatgaacttaatttcaattatttgacacttttaaaaaggattaaaaagactagaaaacaattaaatagcgaaagcattaaaataaacagtaagaatgcacaagaccatttagaaacccttctatctggggctgcagctgcagctgcaacttccgccaataacttggaccttccgactggtggttcaaacctcccagactctaataacttacacttacctggtaacacaggctttttttagatataaacttaataagatctcgatattcattgctcttttttgtagttttgcctttaactctcggttttttgaatttatcattttcctgatgacctttgactgctctttgatctatcctgaacacaagaaaacgaaaagaataaataaaagtagagatatttattatttagcctgtaccgatcactggaaactggattctattaattttacttattcaaaccaagatatctttttcttttttttctttttttttttttttttcagcagtacattcctgaagtgtacacttgccttgtgtattaaatgatgattcgatattataaaacaccatttaagtcgtcattaatttcaatatcgctttcgtctactgaaacaatttaccttgacttcttattttcaggaattc

SEQ ID NO: 85 - DNA, 657 bp, Artificial Sequence
  Description of Artificial Sequence: Synthetic polynucleotide
cagtttatcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacggatt

SEQ ID NO: 86 - DNA, 1470 bp, Artificial Sequence
  Description of Artificial Sequence: Synthetic polynucleotide
cgccgggatcgaagaaatgatggtaaatgaaataggaaatcaaggagcatgaaggcaaaagacaaatataagggtcgaacgaaaaataaagtgaaaagtgttgatatgatgtatttggctttgcggcgccgaaaaaacgagtttacgcaattgcacaatcatgctgactctgtggcggacccgcgctcttgccggcccggcgataacgctgggcgtgaggctgtgcccggcggagttttttgcgcctgcattttccaaggtttaccctgcgctaaggggcgagattggagaagcaataagaatgccggttggggttgcgatgatgacgaccacgacaactggtgtcattatttaagttgccgaaagaacctgagtgcatttgcaacatgagtatactagaagaatgagccaagacttgcgagacgcgagtttgccggtggtgcgaacaatagagcgaccatgaccttgaaggtgagacgcgcataaccgctagagtactttgaagaggaaacagcaatagggttgctaccagtataaatagacaggtacatacaacactggaaatggttgtctgtttgagtacgctttcaattcatttgggtgtgcactttattatgttacaatatggaagggaactttacacttctcctatgcacatatattaattaaagtccaatgctagtagagaaggggggtaacacccctccgcgctcttttccgatttttttctaaaccgtggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactccaagctggccgct

SEQ ID NO: 87 - DNA, 402 bp, Artificial Sequence
  Description of Artificial Sequence: Synthetic polynucleotide
tagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagtttt

SEQ ID NO: 88 - DNA, 41 bp, Artificial Sequence
  Description of Artificial Sequence: Synthetic oligonucleotide
cacacactagtagtaacatgtctcactcagttacaccatcc

SEQ ID NO: 89 - DNA, 36 bp, Artificial Sequence
  Description of Artificial Sequence: Synthetic oligonucleotide
cacacctcgagttaagatgatgcagatctcgatgca

SEQ ID NO: 90 - DNA, 11 bp, Artificial Sequence
  Description of Artificial Sequence: Synthetic oligonucleotide
atccccaaaaa

SEQ ID NO: 91 - DNA, 32 bp, Artificial Sequence
  Description of Artificial Sequence: Synthetic oligonucleotide
cacaccgtctcaggggatgggggtagggtttc

SEQ ID NO: 92 - DNA, 20 bp, Artificial Sequence
  Description of Artificial Sequence: Synthetic oligonucleotide
gccaaggataatggtgttga

SEQ ID NO: 93 - DNA, 39 bp, Artificial Sequence
  Description of Artificial Sequence: Synthetic oligonucleotide
caccgtctcaccccaaaaaaaaagtaatttttactcgtt

SEQ ID NO: 94 - DNA, 20 bp, Artificial Sequence
  Description of Artificial Sequence: Synthetic oligonucleotide
gcagcaattagttggcgaca

SEQ ID NO: 95 - protein, 15 aa, Artificial Sequence
  Description of Artificial Sequence: Synthetic peptide
EETLPPSPKKVIFTR

SEQ ID NO: 96 - protein, 10 aa, Artificial Sequence
  Description of Artificial Sequence: Synthetic peptide
EETLPPSQKK

SEQ ID NO: 97 - DNA, 1516 bp, Artificial Sequence
  Description of Artificial Sequence: Synthetic polynucleotide
ttctagaaacaaaatggaaaaattgcgtgaaatagttagagagggaagaagagttggcgaagaggatcccagaagaattgtacactcatttaaagttggagtcgcgttggttttagttagctcattttactactatcaaccatttggtccatttactgactactttggtataaatgcgatgtgggccgtaatgaccgtcgttgttgtttttgaattttctgtcggagctactttaagtaaaggattaaatagaggtgtcgcaactttagtcgcaggaggcctagcgttaggagcacatcaattggcttcattatcaggaaggactatagaacccattctattggctacttttgtatttgttacagcagcacttgctacctttgttcgttttttcccgagagttaaggctacatttgattatggaatgctaattttcattctaacttttagcttaatttccttatcccagtttagagacgaagaaatattagacttagctgaatcgagattatcaactgtattagttggcggggttagttgtattttaatttccatatttgtttgtccagtttgggccggtcaggacttacattcactattagtttcaaaccttgatactctaagccactttttacaagaattcggtgatgaatatttcgaagcgagaacatatggtaatattaaagttgttgaaaagagaagaagaaaccttgagagatacaaatcagtgctaaactcaaaatccgatgaagattccctagcaaatttcgcaaaatgggaaccaccacatggcaaattcggttttagacatccatggaaacaatatttagtcgtcgcagctttagttagacagtgcgctcatagaatagatgctttaaactcttatattaattcaaattttcaaatcccaatcgatataaaaaagaaattggaagaaccattcaggagaatgtcattagaatctggaaaagcaatgaaagaagcttcaattagtctgaaaaaaatgaccaaatccagcagttacgatatccatataattaatagccaatctgcatgcaaagccttatctaccttgttaaaatctggtatattaaacgacgttgagccattacaaatggtgagtttactaactacagtttctttattaaatgacatagttaacataacagaaaaaataagtgaatctgtgagagaattggcttccgctgctagattcaggaataaaatgaaacctactgaaccaagtgtttccctaaaaaagttagattcaggttctacaggatgtgcaatgccaataaattcaagggatggtgatcatgttgtaaccatattacttagtgacgatgataaagatgatatagatgatgacgatacttcaaatatagtactagacgatgacactattaatgaaaagtctgaagatggtgaaatacatgtacaaaccagttgtgtaagagaggtgggaatgatgcctgaacattcacttggtgtaagaatattgcaaatttaactcgag

SEQ ID NO: 98 - DNA, 1378 bp, Artificial Sequence
  Description of Artificial Sequence: Synthetic polynucleotide
ttctagaaacaaaatggatattgatcatggaagagaaatagatggagaaatggtttctactattgcgtcatgcggcttgttattgcattccttattagcaggtttcgcaagaaaggtcggtggtgctgccagagaagatcccagaagagttgctcattcattaaaagttggtctagcattggctctagtttcagctgtttactttgtaacaccattattcaacgggttaggcgttagtgcaatttgggctgttcttaccgtagtcgtcgttatggagtttaccgtcggtgcaactttaagtaaaggtttaaatagagctttggcaactttagtcgcaggatgtattgctgtcggagcccatcaattagcagaattaacagaacgttgttcagatcaaggggaaccagttatgttgacagtattagttttttttgtcgcatcagcagcaacatttcttagattcattcccgaaatcaaagcaaaatatgactatggcgtaactatttttatactaactttcggtttagttgctgtttcgtcttacagagtggaagaacttattcaattagctcatcaaagattttacacaattgtcgtcggagtatttatatgtctatgcacaacggtatttttatttcctgtttgggccggagaggacgtccataaattagcttcatcaaatttagggaaattagcgcaatttattgaaggtatggaaacaaactgttttggcgaaaacaacatagctatcaatttagaaggaaaagattttttacaagtatacaaatcggttctgaattcaaaggccactgaagattctttatgcacttttgcaagatgggaaccaagacatggtcagtttagatttagacacccctggtctcaatatcaaaaattaggtacactgtgtagacaatgcgcatcatcaatggaagctttagctagttacgttattaccaccacaaagactcaataccccgcagctgcaaatccggaactttcttttaaagtcagaaaaacatgtcacgaaatgtctactcatagtgctaaagttttaagaggtttagaaatggcaatacgtacaatgacagtcccatacttagccaacaatacagtcgtagttgcaatgaaggccgccgagagattaagatcagaattagaagataacgctgcacttttacaggtaatgcatatggctgttactgctacgttacttgccgatttagtcgatagagtcaaagaaatcacagaatgtgttgatgttttagcaagattagcccattttaaaaatcctgaagatgcaaaatacgcaatcgttggtgctttaactagaggaatagatgatcctttgcctgatgtagttatattataactcgag

SEQ ID NO: 99 - DNA, 3609 bp, Artificial Sequence
  Description of Artificial Sequence: Synthetic polynucleotide
actagtaaatatgtctaatgttccagaaactaaagtagatccttcattgtccacaccagaggtccctagtcaaggtttacatagcagattggacaagatgagagctgattcatccatattgggaagtatgaacaaaatattagtggcaaatagaggtgaaatcccaattagaatctttagaaccgcccacgagttatctatgcagactgttgctatctatgcacatgaggacagattgtcaatgcacagattcaaggccgatgaggcttacgtaattggagacagaggaaaatatacacctgtccaagcatacttacaggtggacgagataatcgaaattgccaaggctcatggtgttaacatggtacacccaggatatggtttcttgtccgaaaatagtgagttcgcaagaaaagtcgaagaagctggaatggcctggattggtcctccacataacgttatagacagtgtcggtgacaaggtttcagcaagaaacttagctatcaagaacaatgtacctgtcgtgccaggaaccgatggtcctgttgaggacccaaaggatgccttgaaatttgtagaaaagtacggttatcctgtcattataaaagcagctttcggaggtggaggtagaggtatgagagttgtgagagagggagatgacatcgttgatgcctttaacagagcatccagtgaagctaagactgccttcggtaatggtacatgtttcattgaaagattcttagacaaaccaaaacatatagaggtacaattgttagcagatggacaaggtaatgtcgtgcacttgtttgaaagagattgctctgttcagaggagacatcaaaaggtagtcgaaatcgctccagccaaagacttacctgtcgaggtgagagatgcaattttggacgatgctgttagattagctgaagatgccaagtacagaaacgcaggaaccgctgagttcttggtagacgagcaaaatagacactacttcattgagataaacccaagaatccaggtcgaacatactattacagaggaaataaccggtatcgatattgttgccgcacaaatacagattgctgccggtgcaactttagagcaattgggattaacacaagacaaaatctcaactagaggttttgctattcagtgtagaataaccacagaagatcctgcaaagcaattccaaccagatactggaaaaatcgaagtctacagatctgctggaggtaatggagtaagattggacggtggtaacggatttgccggtgcaattatatcccctcactatgatagtatgttagtcaagtgctcatgttctggcaccacattcgagatagccagaagaaagatgattagagccttggttgagtttagaataagaggagtcaagactaatattccattcttattggcattattgacacatcctacctttatcgaaggaaaatgctggactacattcattgacgatactccatccttatttgacttgatgaccagtcagaacagggctcaaaagttattggcctacttagcagatttatgtgttaatggaacaagtataaaaggtcaggtaggtaaccctaagttaaagtctgaggtcgttatcccagtgttgaagaactccgaaggaaagattgtagattgtagtaaacctgacccagtcggttggagaaatatattagttgaacaaggtcctgaggctttcgccaaggcagtgagaaagaacgatggagttttggtaatggacactacctggagagatgctcatcaatcattattggctacaagagtcagaactaccgacttattggcaattgcaaatgaaacatctcacgctatgtccggtgcctttgcattagagtgctggggaggtgctacttttgacgttgcaatgagattcttgtatgaagatccatgggacagattaagaaagatgagaaaagcagtgccaaatatcccttttcagatgttgttaagaggtgctaatggagtagcctactcatctttgccagataacgcaatagatcatttcgtcaagcaagctaaagacaatggtgttgatatctttagagtgttcgacgccttaaacgatttggatcaattaaaggtaggtgttgacgcagtcaagaaagctggaggtgttgtggaagcaaccgtatgttatagtggagatatgttgaatcctaagaagaagtacaacttagagtattacttggactttgtcgatagagttgtagaaatgggcacccacatcttaggtattaaagatatggcaggaactttgaagccagctgccgcaaccaaattaataggtgctatcagagaaaagtatcctaatttgccaattcatgttcatacacacgactccgccggtactggagtggcatcaatggctgccgcagctgaggccggtgcagatgtcgttgacgtggcttctaatagtatgtctggaatgacctcccagccttcaataagtgccttaatggcaacattggaaggaaaattatctactggtttggacccagctttagtaagagaattggatgcctattgggcacaaatgagattattgtactcatgcttcgaggctgacttaaagggacctgatccagaagtctttcaacatgaaattcctggtggtcagttgacaaacttattgttccaagcccagcaagttggattaggtgagcaatggaaagaaactaagcaggcatatatcgctgccaatcaattgttaggagacattgtaaaagttaccccaacatctaaggtggtcggtgatttggcacagtttatggtttccaacaaattaagttacgacgatgtgataaaacaggctggttcattggattttcctggatctgtattagacttctttgagggtttgatgggtcaaccatatggaggtttcccagaacctttaagaactgaagcattaagaggacagagaaagaaattaaccgagaggcctggaaaatccttgcctccagtcgattttgcagctgttagaaaagacttagaagaaagattcggtcacatcacagagtgtgatattgccagttactgcatgtatcctaaggtatttgaagattacagaaagatagttgacaagtatggagatttgtcaattgtgccaactagattattcttggaagcacctaaaaccgacgaggaattttctgtcgaaatcgagcaaggtaagacattaatattggctttaagagctattggtgatttgtccatgcaaactggattaagagaagtttacttcgagttgaatggtgaaatgagaaagatcagtgtggaagataagaaagccgcagtagaaaccgtgtcaagaccaaaagccgaccctggaaacccaaatgaagttggtgcccctatggccggtgtagttgtggaagtcagagttcatgagggaacagaagtgaagaaaggtgatccagtagctgtcttatctgccatgaagatggaaatggttatttccgccccagtctcaggtaaagtaggagaggtcccagttaaggaaggtgactctgttgatggaagtgatttgatatgcaaaatcgtgagagcttaactcgagctagcgaagacaaccag

SEQ ID NO: 100 - DNA, 3612 bp, Artificial Sequence
  Description of Artificial Sequence: Synthetic polynucleotide
actagtaaatatggctgcaccaagacaacctgaagaggccgttgatgacactgagttcattgatgaccatcacgatcagcatagagacagcgtacacaccagattgagagctaattcagcaataatgcaattccagaaaatcttagtcgccaacagaggtgagattccaataagaatctttagaaccgctcatgaattgtccttacaaactgtggcagtttatagtcacgaagatcatttgtctatgcatagacaaaaggccgatgaggcttacatgattggaaagagaggtcagtatacacctgtaggagcatacttagctatagacgaaatcgtcaagattgccttggaacacggtgtgcacttaattcacccaggttatggattcttgtcagagaatgcagaatttgctagaaaagttgaacaatccggtatggtattcgtcggacctaccccacaaactatagagagtttaggtgataaggtttctgccagacagttggcaatcagatgtgacgtgcctgttgtaccaggtacacctggaccagtcgaaagatacgaggaagtgaaggcttttaccgatacttatggtttccctattataatcaaggccgcatttggtggaggtggaagaggtatgagagttgtaagagatcaagctgaattaagagactcattcgagagagccacatccgaagcaagaagtgcttttggtaacggaaccgtgttcgttgaaagattcttggatagaccaaaacatattgaggtgcagttattgggtgacaatcacggtaacgtggtacacttatttgaaagagattgtagtgtgcaaaggagacatcaaaaggtggttgaaatagcccctgcaaaagatttgccagctgacgtaagagatagaatcttagctgacgccgtcaagttggcaaaatcagttaattacagaaacgctggaactgccgagttcttagtggatcagcaaaatagatattacttcattgaaattaacccaagaatacaagttgaacacaccatcactgaggaaattaccggtatagatatcgtagcagctcagattcaaatagccgcaggagctacattggagcagttaggtttgactcaagacagaatttccaccagaggtttcgcaatccaatgtagaattacaactgaagatcctagtaagggattttctccagacacaggaaaaatagaagtctatagatcagctggtggaaatggtgttagattagatggaggtaatggtttcgccggagcaatcattacccctcattacgattctatgttggtgaaatgcacttgtagaggttccacatatgagatcgccagaagaaaggtagtcagagccttagttgagtttagaatcagaggtgtgaaaactaacattccattcttgacctccttattgtcacaccctgtgtttgtggatggaacatgctggactaccttcatagatgacacaccagaattatttgcattggtcggttctcagaatagggctcaaaagttattggcctacttaggagatgttgcagtgaacggttccagtattaaaggtcaaatcggagagcctaagttgaaaggtgacattataaagccagtattacatgatgctgccggtaaacctttggatgtctcagttccagcaactaagggatggaaacagatcttagactctgaaggtcctgaggcttttgctagagccgtgagagcaaataagggatgtttgattatggataccacatggagggacgctcatcaatccttattggccactagagttagaaccatagacttattgaacattgcacacgagacaagtcatgctttagccaatgcatattcattggaatgttggggtggtgctactttcgatgtagcaatgagattcttatacgaggacccatgggatagattgagaaaattaagaaaagcagtccctaatatcccattccaaatgttgttaagaggagctaatggtgttgcctattcttccttgccagacaacgcaatataccacttttgcaagcaggctaagaagtgtggtgtggatattttcagagtatttgatgccttaaacgacgtcgatcaattggaagttggaatcaaagcagtgcatgctgccgaaggtgtagttgaggcaacaatttgctattcaggagatatgttaaacccttctaagaaatacaacttgccatactacttagatttggtcgataaggttgtgcagttcaaacctcacgtattaggtataaaggatatggctggtgtcttgaaaccacaagccgcaagattattgatcggaagtattagagaaagataccctgacttgcctatacatgttcatacacacgactccgctggtactggtgtagcttcaatgattgcatgtgctcaagccggagcagatgctgttgatgccgcaaccgactctttgagtggtatgacatctcagcctagtatcggagctatcttagcctcattggaaggtactgagcatgatccaggtttaaacagtgcacaagtgagagctttggacacatattgggcccaattaagattgttatactctccttttgaagcaggattgactggtccagatcctgaagtctatgagcacgaaataccaggtggacagttaaccaacttgatcttccaggcttcacagttaggtttgggacaacaatgggccgaaacaaagaaagcatacgagtctgctaatgacttattgggtgacgttgtgaaagtaactcctacctccaaggtcgttggtgacttagcccagtttatggtaagtaacaaattgacagcagaggacgttattgctagagccggagagttagattttccaggttcagtgttggagttcttagaaggtttgatgggacaaccatatggtggatttcctgagccattaagaagtagagcattgagagacagaagaaagttagataaaagacctggtttgtacttagaaccattggacttagctaagatcaaatcccaaattagagaaaattatggtgctgccactgagtacgacgtcgcaagttatgctatgtaccctaaggttttcgaagattataagaagtttgtggccaaattcggagacttgtcagtattaccaaccagatacttcttggcaaagcctgaaatcggtgaggagttccatgtcgaattagagaaaggtaaggttttgatattaaagttgttagctattggaccattgtctgaacagacaggtcaaagagaggtgttttatgaagttaacggagaagtgagacaggtgtccgttgatgataagaaggccagtgtggagaatactgcaagacctaaagctgaattaggtgactcatctcaggtgggagccccaatgtccggagtcgttgtagaaatcagagttcatgatggtttggaggtgaagaaaggtgaccctattgcagtcttatcagctatgaagatggaaatggttatatctgcacctcacagtggaaaagtgtcctcattgttagtaaaggaaggtgattctgtcgatggacaagacttggtttgcaaaatcgtgaaggcttaactcgagctagcgaagacaaccag

SEQ ID NO: 101 - DNA, 3420 bp, Artificial Sequence
  Description of Artificial Sequence: Synthetic polynucleotide
actagtaaatatgttttccaaagttttggtagctaatagaggtgagattgccataagagccttcagagctgcatatgaattaggagccagaactgtcgctgtctttccatacgaagatagatggtcagagcatagattgaaagccgacgaggcttacgagatcggagaaagaggacaccctgttagagcttacttggacccagaagcaattgtagcagtcgccataagagccggtgccgatgcagtgtatcctggttacggtttcttgtccgaaaacccagcattggccgaggcctgtgcaaacgctggtatcacatttgtaggtcctaccgccgatgtattgactttaacaggtaacaaagcaagagcaattgccgcagctaccgctgccggtgtccctactttagcaagtgttgaaccttctactgacgtggacgccttggtggaatcagccggagagttgccatacccattattcgtaaaggcagtggctggtggaggtggtagaggaatgagaagagttgatgcaccaggtcaattgagagaagcagttgagacatgtatgagagaagctgaaggtgcatttggcgaccctactgtattcatagagcaggctgtcgttgatccaagacatatcgaagtgcaagtattggcagacggtgaaggtcacgtaatgcatttgtttgagagagattgttccgtccagaggagacaccagaaagtgattgaaatcgcccctgctccaaacttagacccagagttgagagacagaatatgcgcagacgccgttagattcgctaaggaaatcggatacagaaatgccggtactgtcgagttcttattggacgcaaaaggaacctatcatttcattgaaatgaatcctagaatacaagtcgagcatacagtgactgaagaggtgacagatgtagacttagtacagagtcaattgagaatcgcttctggtgaaaccttagccgacttgggattatcacaagaaactgtaaccttgagaggagctgcattgcagtgtagaattactacagaggacccagctaacaactttagacctgacactggtgttatcacaacttacagatccccaggaggtggaggagtgagattggatggtggtactgtgtatactggtgccgaagtcagtgcccactttgattctatgttagctaagttgacttgcagaggtagaaccttcgagaaagccgttgagaaggcaagaagagctgtggccgagtttagaatcagaggtgtttcaacaaacattcctttcttgcaagccgtattggtggacccagacttttccagtggacatgttactacctctttcattgaaacacacccacaattattgcaagccagatcatctggtgacagaggaagcagattgttgcattacttagccgatgtgactgtgaatcaaccacacggtcctgcacctgtttccatcgaccctgttaccaaattgccagaggtgaacttagacgttcctgctccagatggtacaagacagttgttgttagatgttggaccagaagagtttgccagaagattaagagcacaaactggtgttgctgtaaccgatacaactttcagggacgcccatcaatcattgttagctaccagagtgagaacaagagatttgttagctgtagccggtcatgtcgcaagaactacccctcagttgtggtctttagaggcttggggaggtgccacatatgatgtagccttaagattcttagctgaggacccatgggagagattggcagccttaagacaagcagtgcctaacatctgtttgcagatgttattgagaggaagaaatactgtaggttacacaccttatccagccgatgttactcaagcattcgtcgaagaagctgccgcaaccggtattgacgtgtttagaatatttgatgctttaaacgatgtggagcaaatgaggccagccatagaggctgtaagagctacaggaactgccgtcgcagaagttgcattgtgttacacaggagacttatccgatcctgacgagacattgtatactttagattactatttggaattagccgatagaattgtagacgccggagcacacgtcttagctataaaggatatggcaggattattgagagtgccagctgccagaaccttagtcacagcattgagagacagattcgacttgccagttcatttgcacactcatgataccccaggtggacagttagctacattattggcagccattgacgccggtgtggatgctgtagacgccgcaactgctagtatggcaggaacaacatcacaacctccattgtctgcattagtttccgctactgatcatggacctagagaaaccggtttgagtttaggtgccgtgtcagcattggagccatattgggaagctacaagaagagtatacgcacctttcgagtctggattaccttccccaactggtagagtttatagacacgaaatccctggaggtcaattgtcaaacttaagacagcaagctatcgccttaggtttgggagagaaattcgagcaaatagaagatatgtacgcagctgccaacgacatattaggtaatgtggtcaaggttaccccatctagtaaggtagtaggtgacttagcattgcacttagtcgctgttggagccgaccctacagaatttgcagatgagccaggaaaattcgatattcctgactccgtaataggattcttaaatggagaattgggtgacccacctggaggttggccagaacctttcagaactaaggccttagctggtagaactcacaagcctcctgttgaggaattagacgatgaacagagagagggattggccggttcatctccaacaagaagaagaactttaaacgaattgttatttccaggtccaacaaaggagttcacagaaagtagattaagatatggtgacacttctgtgttaccaacattggattacttatatggtttgagaagaggagaagagcatgcagtcgaaatcgaagagggtaaaacattaatcttgggagttcaagccataactgaacctgatgaaagaggattcagaaccgtgatgacaactattaacggtcagttaagaccagtgagtgtcagagacagatcagttgccgctgaggttgctgccgcagaaaaggcagataccagtaaacctggacacgttgcagccccatttcaaggtgtggtgtctatcgttgtggaggaaggtcaacaggtagccgctggagacacagtagcaactatcgaagccatgaagatggaggcctcaataaccgcacctgttgccggaacagttgagagattggccttatctggtactcaagcagtagaaggaggtgatttggtcttagttttgtcctaactcgagctagcgaagacaaccag

SEQ ID NO: 102 - DNA, 22 bp, Artificial Sequence
  Description of Artificial Sequence: Synthetic primer
atgaatgaacaatattccgcca

SEQ ID NO: 103 - DNA, 20 bp, Artificial Sequence
  Description of Artificial Sequence: Synthetic primer
ttagccggtattgcgcatcc

SEQ ID NO: 104 - DNA, 1220 bp, Artificial Sequence
  Description of Artificial Sequence: Synthetic polynucleotide
ttctagaaacaaaatgtttaataacgagcatcatatacctcctggatctagccactcggatattgaaatgttaactcctcctaaatttgaagatgaaaaacaacttggacctgtcggtataagagaaagacttagacactttacttgggcttggtatacactaactatgagtgggggcggcttagctgttttaataatttcacaaccttttggtttcagaggtcttagggaaatcggaatcgctgtttatattctaaatcttatactttttgctttagtttgttccactatggctattaggtttatactacatggtaatttattagaaagtttgcgtcatgatagagaaggtttgttctttcccacattctggctttcagttgcaacaattatatgtggtttatcaaggtatttcggtgaagaatcaaatgaaagttttcagctagctttagaagctctgttctggatttattgcgtttgtacactattagtagctattatacaatattcattcgttttctcctctcataaatatggtctacaaactatgatgccatcttggatactaccagcttttcctataatgttgtcaggtactattgcgtctgttattggcgagcaacaaccagctagagcagctttacctataatcggagcaggtgtaacttttcaaggattaggtttttcaatttcttttatgatgtatgcacactatattggtcgtctaatggaatctggtttaccacactcagatcatagacctggtatgtttatatgtgttggtccaccggcctttacagcactagccttagtcggtatgtctaagggtttgcctgaagattttaagttattacatgatgcacacgccctggaagatggaagaattatagaactattagcaatctctgcaggtgttttcttatgggctttaagtttatggtttttttgtattgcaattgtcgccgttatcagatcacctcccaaagcctttcatttaaactggtgggctatggttttcccaaacactggtttcactttagcaacaataaccctaggtaaagcattaaactctaacggtgtaaaaggtgttggttcagctatgagtatttgtattgtatgtatgtatatattcgttttcgtaaataatgttagagctgtgatacgtaaagatataatgtaccctggtaaagacgaagatgtctctgattagtcttctcgag

SEQ ID NO: 105 - protein, 398 aa, Artificial Sequence
  Description of Artificial Sequence: Synthetic polypeptide
MFNNEHHIPPGSSHSDIEMLTPPKFEDEKQLGPVGIRERLRHFTWAWYTLTMSGGGLAVLIISQPFGFRGLREIGIAVYILNLILFALVCSTMAIRFILHGNLLESLRHDREGLFFPTFWLSVATIICGLSRYFGEESNESFQLALEALFWIYCVCTLLVAIIQYSFVFSSHKYGLQTMMPSWILPAFPIMLSGTIASVIGEQQPARAALPIIGAGVTFQGLGFSISFMMYAHYIGRLMESGLPHSDHRPGMFICVGPPAFTALALVGMSKGLPEDFKLLHDAHALEDGRIIELLAISAGVFLWALSLWFFCIAIVAVIRSPPKAFHLNWWAMVFPNTGFTLATITLGKALNSNGVKGVGSAMSICIVCMYIFVFVNNVRAVIRKDIMYPGKDEDVSD

SEQ ID NO: 106 - DNA, 37 bp, Artificial Sequence
  Description of Artificial Sequence: Synthetic oligonucleotide
cacacactagtaaaatatgagcagtagcaagaaattg

SEQ ID NO: 107 - DNA, 3592 bp, Artificial Sequence
  Description of Artificial Sequence: Synthetic polynucleotide
actagtaaatatgtctaatgttccagaaactaaagtagatccttcattgtccacaccagaggtccctagtcaaggtttacatagcagattggacaagatgagagctgattcatccatattgggaagtatgaacaaaatattagtggcaaatagaggtgaaatcccaattagaatctttagaaccgcccacgagttatctatgcagactgttgctatctatgcacatgaggacagattgtcaatgcacagattcaaggccgatgaggcttacgtaattggagacagaggaaaatatacacctgtccaagcatacttacaggtggacgagataatcgaaattgccaaggctcatggtgttaacatggtacacccaggatatggtttcttgtccgaaaatagtgagttcgcaagaaaagtcgaagaagctggaatggcctggattggtcctccacataacgttatagacagtgtcggtgacaaggtttcagcaagaaacttagctatcaagaacaatgtacctgtcgtgccaggaaccgatggtcctgttgaggacccaaaggatgccttgaaatttgtagaaaagtacggttatcctgtcattataaaagcagctttcggaggtggaggtagaggtatgagagttgtgagagagggagatgacatcgttgatgcctttaacagagcatccagtgaagctaagactgccttcggtaatggtacatgtttcattgaaagattcttagacaaaccaaaacatatagaggtacaattgttagcagatggacaaggtaatgtcgtgcacttgtttgaaagagattgctctgttcagaggagacatcaaaaggtagtcgaaatcgctccagccaaagacttacctgtcgaggtgagagatgcaattttggacgatgctgttagattagctgaagatgccaagtacagaaacgcaggaaccgctgagttcttggtagacgagcaaaatagacactacttcattgagataaacccaagaatccaggtcgaacatactattacagaggaaataaccggtatcgatattgttgccgcacaaatacagattgctgccggtgcaactttagagcaattgggattaacacaagacaaaatctcaactagaggttttgctattcagtgtagaataaccacagaagatcctgcaaagcaattccaaccagatactggaaaaatcgaagtctacagatctgctggaggtaatggagtaagattggacggtggtaacggatttgccggtgcaattatatcccctcactatgatagtatgttagtcaagtgctcatgttctggcaccacattcgagatagccagaagaaagatgattagagccttggttgagtttagaataagaggagtcaagactaatattccattcttattggcattattgacacatcctacctttatcgaaggaaaatgctggactacattcattgacgatactccatccttatttgacttgatgaccagtcagaacagggctcaaaagttattggcctacttagcagatttatgtgttaatggaacaagtataaaaggtcaggtaggtaaccctaagttaaagtctgaggtcgttatcccagtgttgaagaactccgaaggaaagattgtagattgtagtaaacctgacccagtcggttggagaaatatattagttgaacaaggtcctgaggctttcgccaaggcagtgagaaagaacgatggagttttggtaatggacactacctggagagatgctcatcaatcattattggctacaagagtcagaactaccgacttattggcaattgcaaatgaaacatctcacgctatgtccggtgcctttgcattagagtgctggggaggtgctacttttgacgttgcaatgagattcttgtatgaagatccatgggacagattaagaaagatgagaaaagcagtgccaaatatcccttttcagatgttgttaagaggtgctaatggagtagcctactcatctttgccagataacgcaatagatcatttcgtcaagcaagctaaagacaatggtgttgatatctttagagtgttcgacgccttaaacgatttggatcaattaaaggtaggtgttgacgcagtcaagaaagctggaggtgttgtggaagcaaccgtatgttatagtggagatatgttgaatcctaagaagaagtacaacttagagtattacttggactttgtcgatagagttgtagaaatgggcacccacatcttaggtattaaagatatggcaggaactttgaagccagctgccgcaaccaaattaataggtgctatcagagaaaagtatcctaatttgccaattcatgttcatacacacgactccgccggtactggagtggcatcaatggctgccgcagctgaggccggtgcagatgtcgttgacgtggcttctaatagtatgtctggaatgacctcccagccttcaataagtgccttaatggcaacattggaaggaaaattatctactggtttggacccagctttagtaagagaattggatgcctattgggcacaaatgagattattgtactcatgcttcgaggctgacttaaagggacctgatccagaagtctttcaacatgaaattcctggtggtcagttgacaaacttattgttccaagcccagcaagttggattaggtgagcaatggaaagaaactaagcaggcatatatcgctgccaatcaattgttaggagacattgtaaaagttaccccaacatctaaggtggtcggtgatttggcacagtttatggtttccaacaaattaagttacgacgatgtgataaaacaggctggttcattggattttcctggatctgtattagacttctttgagggtttgatgggtcaaccatatggaggtttcccagaacctttaagaactgaagcattaagaggacagagaaagaaattaaccgagaggcctggaaaatccttgcctccagtcgattttgcagctgttagaaaagacttagaagaaagattcggtcacatcacagagtgtgatattgccagttactgcatgtatcctaaggtatttgaagattacagaaagatagttgacaagtatggagatttgtcaattgtgccaactagattattcttggaagcacctaaaaccgacgaggaattttctgtcgaaatcgagcaaggtaagacattaatattggctttaagagctattggtgatttgtccatgcaaactggattaagagaagtttacttcgagttgaatggtgaaatgagaaagatcagtgtggaagataagaaagccgcagtagaaaccgtgtcaagaccaaaagccgaccctggaaacccaaatgaagttggtgcccctatggccggtgtagttgtggaagtcagagttcatgagggaacagaagtgaagaaaggtgatccagtagctgtcttatctgccatgaagatggaaatggttatttccgccccagtctcaggtaaagtaggagaggtcccagttaaggaaggtgactctgttgatggaagtgatttgatatgcaaaatcgtgagagcttaactcgag

(19) United States
(12) Patent Application Publication
(10) Pub. No.: US 2011/0045559 A1
(43) Pub. Date: Feb. 24, 2011

(54) MALIC ACID PRODUCTION IN RECOMBINANT YEAST

(76) Inventors: Aaron Adriaan Winkler, The Hague (NL); Abraham Frederik De Hulster, Pijnacker (NL); Johannes Pieter Van Dijken, Leidschendam (NL); Jacobus Thomas Pronk, Schipluiden (NL); Joshua Trueheart, Concord, MA (US); Kevin T. Madden, Arlington, MA (US); Jacob C. Harrison, Newton, MA (US); Carlos Gancedo Rodriguez, Majadahonda (ES); Carmen-Lisset Flores Mauriz, Madrid (ES)

Correspondence Address: FISH & RICHARDSON P.C. (BO), P.O. BOX 1022, MINNEAPOLIS, MN, 55440-1022, US

(21) Appl. No.: 12/600,537

(22) PCT Filed: May 19, 2008
(86) PCT No.: PCT/US08/64078

§ 371 Date: Nov. 9, 2010

Related U.S. Application Data
(60) Provisional application No. 60/938,829, filed on May 18, 2007.

(51) Int. Cl. C12P 7/46; C12N 1/19

(52) U.S. Cl. 435/145; 435/254.2

(57) ABSTRACT

The present disclosure relates to modified yeast, wherein the yeast has reduced pyruvate decarboxylase polypeptide (PDC) activity and methods of using such yeast to produce malic and/or succinic acid.

### BACKGROUND

Dicarboxylic acids are organic compounds that include two carboxylic acid groups. Such compounds find utility in a variety of commercial settings including, for example, in areas relating to food additives, polymer plasticizers, solvents, lubricants, engineered plastics, epoxy curing agents, adhesive and powder coatings, corrosion inhibitors, cosmetics, pharmaceuticals, electrolytes, etc.

Carboxylic acid groups, including those in dicarboxylic acids, are readily convertible into their ester forms. Such carboxylic acid esters are commonly employed in a variety of settings. For example, lower chain esters are often used as flavouring base materials, plasticizers, solvent carriers and/or coupling agents. Higher chain compounds are commonly used as components in metalworking fluids, surfactants, lubricants, detergents, oiling agents, emulsifiers, wetting agents textile treatments and emollients.

Carboxylic acid esters are also used as intermediates for the manufacture of a variety of target compounds. A wide range of physical properties (e.g., viscosities, specific gravities, vapor pressures, boiling points, etc.) can be achieved with different esters of the same carboxylic acid. It is therefore desirable to develop production systems for dicarboxylic acid compounds and/or their esters.

The use of microorganisms, such as yeast, in performing industrial processes has taken place serendipitiously for thousands of years and has been a subject of technical inquiry for decades. Certain yeasts, for example, S. cerevisiae have been used to produce many different small molecules, including some organic acids.

However, one organic acid that has been difficult to produce from yeast, particularly S. cerevisiae, is malic acid. Malic acid, C₄H₆O₅, is a dicarboxylic organic acid that imparts a tart taste to many sour or tart foods, such as green apples and wine. Malic acid is useful to the food processing industry as a source of tartness for use in various foods. There remains a need for the development of improved systems, and in particular for the development of improved microbiological systems, for the production of malic acid.

Succinic acid is a useful compound that can be produced, for example in yeast, from malate. Succinic acid has many uses: surfactant/detergent/extender/foaming agent, ion chelator, acidulant/pH modifier, a flavoring agent (e.g., in the form of sodium succinate), and/or an anti-microbial agent. Succinic acid can also be employed as a feed additive. Succinic acid can be utilized to improve the properties of soy proteins in food or feed through the succinylation of lysine residues. Succinic acid also finds utility in the pharmaceutical/health products market, for example in the production of pharmaceuticals (including antibiotics), amino acids, vitamins, etc. Succinic acid can also be utilized to modify other compounds and thereby to improve or adjust their properties. For example, succinylation of proteins (e.g., on lysine residues) can improve their physical or functional attributes; succinylation of cellulose can improve water absorbitivity; succinylation of starch can enhance its utility as a thickening agent, etc.

### SUMMARY OF THE DISCLOSURE

In certain embodiments, the present disclosure relates, to a modified (e.g., recombinant) yeast, wherein the yeast has reduced pyruvate decarboxylase enzyme (PDC) activity (i.e., is PDC-reduced or PDC-negative) and is functionally transformed to increase the activity of one more polypeptides chosen from a pyruvate carboxylase (PYC) polypeptide, a phosphoenolpyruvate carboxylase (PPC) polypeptide, a malate dehydrogenase (MDH) polypeptide, and an organic acid transport (MAE) polypeptide.

In some embodiments, the recombinant yeast is functionally transformed to increase the activity of a PYC polypeptide or a PPC polypeptide, together with modifications to increase the activities of a MDH polypeptide and a MAE polypeptide.

In some embodiments, the modified (e.g., recombinant) PDC-reduced yeast is functionally transformed to increase the activity of a PYC polypeptide that is active in the cytosol. In some embodiments, the recombinant PDC-reduced yeast that is functionally transformed to increase the activity of a PPC polypeptide is modified to be less sensitive to inhibition by one more of malate, aspartate, and oxaloacetate. For example, the PPC polypeptide has one or more amino acid changes that reduce (compared to an otherwise identical PPC polypeptide lacking the one or more amino acid changes) the feedback inhibition caused by the presence of one more of malate, aspartate, and oxaloacetate. In some embodiments, the recombinant PDC-reduced yeast is functionally transformed to increase the activity of a MDH polypeptide such the the MDH polypeptide exhibits increased activity in the cytosol and/or is less sensitive to inactivation in the presence of glucose. For example, the recombinant PDC-reduced yeast can have a genetic modification in a MDH polypeptide-encoding gene or elsewhere that increases the level of MDH polypeptide in the cytosol compared to an otherwise identical yeast. This can be achieved, for example, by a genetic change that causes a higher proportion of the MDH polypeptide present in the yeast to be located in the cytosol relative to one or more other compartments in the cell. In another example, the MDH polypeptide can have one or more amino acid changes that reduce (compared to an otherwise identical MDH polypeptide lacking the one or more amino acid changes) the feedback inhibition caused by the presence of glucose.

In certain embodiments, the, present disclosure relates to a method of producing malic acid or succinic acid including culturing a modified (e.g., recombinant) yeast, wherein the yeast has reduced pyruvate decarboxylase enzyme (PDC) activity (i.e., is PDC-reduced or PDC-negative) and is functionally transformed to increase the activity of either a pyruvate carboxylase (FTC) polypeptide, a phosphoenolpyruvate carboxylase polypeptide (PPC), a malate dehydrogenase (MDH) polypeptide, and/or an organic acid transport (MAE) polypeptide. In some embodiments, the recombinant yeast is functionally transformed to increase the activity of a PYC polypeptide or a PPC polypeptide, together with a modification to increase the activity of a MDH polypeptide and a MAE polypeptide.

Such a modified (e.g., recombinant) yeast may be cultured under conditions that allow production of malic acid and/or succinic acid, and such produced acid may be isolated from the medium. In some embodiments, the yeast is cultured in a medium comprising a carbon source and a carbon dioxide source.

In certain embodiments, the present disclosure provides food products comprising malic acid and/or succinic acid produced by the modified yeast described herein. In further embodiments, the present disclosure provides cosmetics comprising malic acid and/or succinic acid produced by the modified yeast described herein. In other embodiments, the present disclosure provides industrial chemicals such as surfactants, monomers such as 1,4-butanediol or tetrahydrofuran for biobased polymers, or biodegradable polymers comprising malic acid and/or succinic acid produced by the modified yeast described herein.

Described herein are modified yeast having a genetic modification that reduces pyruvate decarboxylase (PDC) polypeptide activity compared to an otherwise identical yeast lacking the genetic modification and at least one modification (e.g., an additional genetic modification) that increases malic acid production as compared with an otherwise identical yeast lacking the modification. In various embodiments: the PDC polypeptide activity of the modified yeast is approximately 3 fold less than PDC polypeptide activity exhibited by an otherwise identical yeast lacking the genetic modification; the PDC polypeptide activity of the modified yeast is approximately 5 fold less than PDC polypeptide activity exhibited by an otherwise identical yeast lacking the genetic modification; the PDC polypeptide activity of the modified yeast is approximately 10 fold less than PDC polypeptide activity exhibited by an otherwise identical yeast lacking the genetic modification; the PDC polypeptide activity'of the modified yeast is approximately 50 fold less than PDC polypeptide activity exhibited by an otherwise identical yeast lacking the genetic modification; the modified yeast exhibits PDC polypeptide activity of less than about 0.075 micromol/min mg protein-1; the modified yeast exhibits PDC polypeptide activity of less than about 0.045 micromol/min mg protein-1; the modified yeast exhibits PDC polypeptide activity of less than about 0.025 micromol/min mg protein-1; the modified yeast exhibits PDC polypeptide activity of less than about 0.005 micromol/min mg protein-1; and the modified yeast exhibits no detectable PDC polypeptide activity.

In some cases: the modification that increases malic acid production as compared with an otherwise identical yeast lacking the modification comprises at least one chemical, physiological, or genetic modification; the yeast is of a genus selected from the group consisting of Saccharomyces, Zygosaccharomyces, Candida, Hansenula, Kluyveromyces, Debaromyces, Nadsonia, Lipomyces, Torulopsis, Kloeckera, Pichia, Schizosaccharomyces, Trigonopsis, Brettanomyces, Cryptococcus, Trichosporon, Aureobasidium, Lipomyces, Phaffia, Rhodotorula, Yarrowia, or Schwanniomyces; the yeast is a strain of S. cerevisiae selected from the group consisting of TAM, Lp4f, m850, RWB837, and strains derived from TAM, Lp4f, m850, DV10, and RWB837; and the yeast is of a species selected from the group consisting of: Kluyveromyces lactis, Saccharomyces cerevisiae var bayanus, Saccharomyces boulardii, and Zygosaccharomyces bailii.

In some cases, the reduced PDC polypeptide activity is conferred by: a genetic modification that deletes at least a portion of a gene encoding a PDC polypeptide, a genetic modification that alters the sequence of a gene encoding a PDC polypeptide, a genetic modification that disrupts a gene encoding a PDC polypeptide, or a genetic modification that reduces the transcription or translation of gene or RNA encoding a PDC polypeptide; reduced PDC polypeptide activity is conferred by a modification selected from the group consisting of modifications that decrease one or more of PDC1, PDC2, PDC5 and PDC6 activities; the modification to decrease PDC polypeptide activity comprises modifications to decrease each of PDC1, PDC5, and PDC6 activities; the modification to decrease PDC polypeptide activity comprises modifications to decrease each of PDC1 and PDC5 activities; the PDC polypeptide has an amino acid sequence identical to that of a PDC polypeptide from an organism of the Saccharomyces genus; wherein the PDC polypeptide has an amino acid sequence identical to that of a Saccharomyces cerevisiae PDC polypeptide; the yeast harbors a nucleic acid sequence encoding a PDC1 protein having at least 75% identity to SEQ ID NO:77; the yeast harbors a nucleic acid sequence encoding a PDC1 protein having at least 95% identity to SEQ ID NO:77; the yeast harbors a nucleic acid sequence encoding a PDC5 protein having at least 75% identity to SEQ ID NO:79; the yeast harbors a nucleic acid sequence encoding a PDC5 protein having at least 95% identity to SEQ ID NO:79; the yeast harbors a nucleic acid sequence encoding a PDC6 protein having at least 75% identity to SEQ ID NO:81; the yeast harbors a nucleic acid sequence encoding a PDC6 protein having at least 95% identity to SEQ ID NO:81; the yeast harbors a nucleic acid sequence encoding a PDC2 protein having at least 75% identity to SEQ ID NO:83; the yeast harbors a nucleic acid sequence encoding a PDC2 protein of at least 95% identity to SEQ ID NO:83; the PDC polypeptide has an amino acid sequence identical to that of a a PDC polypeptide in FIG. 20; the PDC polypeptide has at least 75% identity to a PDC polypeptide in FIG. 20; the PDC polypeptide has at least 95% identity to a PDC polypeptide in FIG. 20.

In some cases: the at least one modification that increases malic acid production comprises a genetic modification that increases activity of at least one polypeptide selected from the group consisting of: a pyruvate carboxylase (PYC) polypeptide, a phosphoenolpyruvate carboxylase (PPC) polypeptide, a malate dehydrogenase (MDH) polypeptide, an organic acid transport (MAE) polypeptide, and combinations thereof as compared with its activity in an otherwise identical yeast lacking the modification.

In some cases: the at least one modification comprises a genetic modification that increases activity of a PYC polypeptide; the at least one modification increases activity by increasing expression of the PYC polypeptide to a level above that at which it is expressed in an otherwise identical yeast that lacks the at least one modification; the PYC polypeptide is active in the cytosol; the genetic modification is the addition of a gene encoding a PYC polypeptide; the genetic modification is a genetic modification of a gene encoding a PYC polypeptide that increases transcription or translation of the gene or a genetic modification that alters the coding sequence of a gene encoding a PYC polypeptide; the PYC polypeptide is heterologous to the yeast; the PYC polypeptide has an amino acid sequence identical to that of a PYC polypeptide from an organism of the Saccharomyces genus; the PYC polypeptide has an amino acid sequence identical to that of a Saccharomyces cerevisiae PYC polypeptide; the PYC polypeptide has at least 75% identity to SEQ ID NO:1 (PYC2); the PYC polypeptide has at least 95% identity to SEQ ID NO:1 (PYC2); the PYC polypeptide has at least 75% identity'to SEQ ID NO:61 (Saccharomyces cerevisiae PYC1); the PYC polypeptide has at least 95% identity to SEQ ID NO:61 (Saccharomyces cerevisiae PYC1); the PYC polypeptide has an amino acid sequence identical to that of a PYC2-ext polypeptide; the PYC polypeptide has at least 75% identity to SEQ ID NO:65 (PYC2-ext); the PYC polypeptide has at least 95% identity to SEQ ID NO:65 (PYC2-ext); the PYC polypeptide has an amino acid sequence identical to that of a Y. lipolytica PYC1 polypeptide; the PYC polypeptide has at least 75% identity to SEQ ID NO:67 (Y. lipolytica PYC 1); the PYC polypeptide has at least 95% identity to SEQ ID NO:67(Y. lipolytica PYC1); the PYC polypeptide has an amino acid sequence identical to that of an A. niger pycA polypeptide; the PYC polypeptide has at least 75% identity to SEQ ID NO:69 (A. niger pycA); the PYC polypeptide has at least 95% identity to SEQ ID NO:69 (A. niger pycA); the PYC polypeptide has an amino acid sequence identical to that of a Nocardia sp. JS614 pycA polypeptide; the PYC polypeptide has at least 75% identity to SEQ ID NO:71 (Nocardia sp. JS614 pycA); the PYC polypeptide has at least 95% identity to SEQ. ID NO:71 (Nocardia sp. JS614 pycA); the PYC polypeptide has an amino acid sequence identical to that of a Methanothermobacter thermautotrophicus str. Delta H pycA polypeptide; the PYC polypeptide has at least 75% identity to SEQ ID NO:73 (Methanothermobacter thermautotrophicus str. Delta H pycA); PYC polypeptide has at least 95% identity to SEQ ID NO:73 (Methanothermobacter thermautotrophicus str. Delta H pycA); the. PYC polypeptide has an amino acid sequence identical to that of a Methanothermobacter thermautotrophicus str. Delta H pycB polypeptide; the PYC polypeptide has at least 75% identity to SEQ ID NO:75 (Methanothermobacter thermautotrophicus str. Delta H pycB); the PYC polypeptide has at least 95% identity to SEQ ID NO:75 (Methanothermobacter thermautotrophicus str. Delta H pycB); the PYC polypeptide has an amino acid sequence identical to that of a a PYC polypeptide in FIG. 22; the PYC polypeptide has at least 75% identity to a PYC polypeptide in FIG. 22; and the PYC polypeptide has at least 95% identity to a PYC polypeptide in FIG. 22.

In some cases: the at least one modification comprises a genetic modification that increases the activity of a phosphoenol pyruvate carboxylase (PPC) polypeptide as compared with its activity in an otherwise identical yeast lacking the modification; the modification increases activity of the PPC by increasing its expression; the yeast contains a modification to decrease sensitivity of the PPC polypeptide to inhibition by one more of malate, aspartate, and oxaloacetate; the genetic modification is the addition of a gene encoding a PPC polypeptide; the genetic modification is a genetic modification of a gene encoding a PPC polypeptide that increases transcription or translation of the gene or a genetic modification that alters the coding sequence of a gene encoding a PPC polypeptide; the PPC polypeptide is heterologous to the yeast; the PPC polypeptide has an amino acid sequence identical to that of a PPC polypeptide from an organism of the Escherichia genus; the PPC polypeptide has an amino acid sequence identical to that of an Escherichia coli PPC polypeptide; the PPC polypeptide has at least 75% identity to SEQ ID NO:7 (E. coli PPC); the PPC polypeptide has at least 95% identity to SEQ ID NO:7 (E. coli PPC); the PPC polypeptide has an amino acid sequence identical to that of an Escherichia coli mut5-K620S Ppc polypeptide; the PPC polypeptide has at least 75% identity to SEQ ID NO:51 (Escherichia coli mut5-K620S Ppc); the PPC polypeptide has at least 95% identity to SEQ ID NO:51 (Escherichia coli mut5-K620S F′pc); the PPC polypeptide has an amino acid sequence identical to that of an Escherichia coli mut10-K773G Ppc polypeptide; the PPC polypeptide has at least 75% identity to SEQ ID NO:53 (Escherichia coli mut10-K773G Ppc); the PPC polypeptide has at least 95% identity to SEQ 1D NO:53 (Escherichia coli mut10-K773G Ppc); the PPC polypeptide has an amino acid sequence identical to that of an Erwinia carotovora Ppc polypeptide; the PPC polypeptide has at least 75% identity to SEQ ID NO:55 (Erwinia carotovora Ppc); the PPC polypeptide has at least 95% identity to SEQ ID NO:55 (Erwinia carotovora Ppc); the PPC polypeptide has an amino acid sequence identical to that of a (Thermo)synechococcus vulcanus Ppc polypeptide; the PPC polypeptide has at least 75% identity to SEQ ID NO:57 ((Thermo)synechococcus vulcanus Ppc); the PPC polypeptide has at least 95% identity to SEQ ID NO:57 ((Thermo)synechococcus vulcanus Ppc); the PPC polypeptide has an amino acid sequence identical to that of a Corynebacterium glutamicum Ppc polypeptide; the PPC polypeptide has at least 75% identity to SEQ ID NO:59 (Corynebacterium glutamicum Ppc); the PPC polypeptide has at least 95% identity to SEQ ID NO:59 (Corynebacterium glutamicum Ppc); the PPC polypeptide has an amino acid sequence identical to a PPC polypeptide in FIG. 21; the PPC polypeptide has at least 75% identity to a PPC polypeptide in FIG. 21; and the PPC polypeptide has at least 95% identity to a PPC polypeptide in FIG. 21

In some cases: the at least one modification comprises a genetic modification that increases activity of an MDH polypeptide; the genetic modification increases activity by increasing expression of the MDH; the genetic modification is the addition of a gene encoding a MDH polypeptide; the genetic modification is a genetic modification of a gene encoding a MDH polypeptide that increases transcription or translation of the gene or a genetic modification that alters the coding sequence of a gene encoding a MDH polypeptide the MDH polypeptide is active in the cytosol; the MDH polypeptide is targeted to the cytosol of the yeast by modification of its coding region; the yeast contains a modification that decreases sensitivity of the MDH polypeptide to inhibition in the presence of glucose; the modified yeast has at least 2-fold the MDH polypeptide activity in the presence of glucose, when compared to an otherwise identical parental strain lacking the modification that decreases sensitivity of the MDH polypeptide to inhibition in the presence of glucose; the modification that decreases sensitivity of the MDH polypeptide to inhibition in the presence of glucose is a change in the coding sequence of a gene encoding a MDH polypeptide; the MDH polypeptide is heterologous to the yeast; the MDH polypeptide has an amino acid sequence identical to that of an MDH polypeptide from an organism of the Saccharomyces genus; the MDH polypeptide has an amino acid sequence identical to that of a Saccharomyces cerevisiae MDH polypeptide; the MDH polypeptide is selected from the group consisting of: MDH1, MDH2, MDH2 P2S or MDH3 and combinations thereof; the MDH polypeptide has an amino acid sequence identical to that of an S. cerevisiae MID1 polypeptide; the MDH polypeptide has at least 75% identity to SEQ ID NO:9 (S.c. MDH1); the MDH polypeptide has at least 95% identity to SEQ ID NO:9 (S.c. MID1); the MDH polypeptide has an amino acid sequence identical to that of an S. cerevisiae MDH2 polypeptide; the MDH polypeptide has at least 75% identity to SEQ ID NO:11 (S.c. MDH2); the MDH polypeptide has at least 95% identity to SEQ ID NO:11 (S.c. MDH2); the MDH polypeptide has an amino acid sequence identical to that of an S. cerevisiae MDH2 P2S polypeptide; the MDH polypeptide has at least 75% identity to SEQ ID NO:13 (S.c. MDH2 P2S); the MDH polypeptide has at least 95% identity to SEQ ID NO:13 (S.c. MDH2 P2S); the MDH polypeptide has an amino acid sequence identical to that of an S. cerevisiae MDH3 polypeptide; the MDH polypeptide has at least 75% identity to SEQ ID NO:15 (S.c. MDH3); the MDH polypeptide has at least 95% identity to SEQ ID NO:15 (S.c. MDH3); the MDH polypeptide has an amino acid sequence identical to that of an S. cerevisiae MDH3ΔSKL polypeptide; the MDH polypeptide has at least 75% identity to SEQ ID NO:17 (S.c. MDH3ΔSKL); the MDH polypeptide has at least 95% identity to SEQ ID NO:17 (S.c. MDH3ΔSKL); the MDH polypeptide has an amino acid sequence identical to that of an Actinobacillus succinogenes MDH polypeptide; the MDH polypeptide has at least 75% identity to SEQ ID NO:19 (Actinobacillus succinogenes MDH); the MDH polypeptide has at least 95% identity to SEQ ID NO:19 (Actinobacillus succinogenes MDH); the MDH polypeptide has an amino acid sequence identical to that of a Yarrowia lipolytica MDH polypeptide; the MDH polypeptide has at least 75% identity to SEQ ID NO:21 (Yarrowia lipolytica MDH); the MDH polypeptide has at least 95% identity to SEQ ID NO:21 (Yarrowia lipolytica MDH); the MDH polypeptide has an amino acid sequence identical to that of an Aspergillus niger MDH polypeptide; the MDH polypeptide has at least 75% identity to SEQ ID NO:23 (Aspergillus niger MDH); the MDH polypeptide has at least 95% identity to SEQ ID NO:23 (Aspergillus niger MDH); the MDH polypeptide has an amino acid sequence identical to that of an MDH polypeptide in FIG. 23; the MDH polypeptide has at least 75% identity to a MDH polypeptide in FIG. 23; the MDH polypeptide has at least 95% identity to a MDH polypeptide in FIG. 23.

In some cases: the at least one modification comprises a genetic modification that increases activity of an organic acid transport polypeptide; the at least one genetic modification increases activity of an organic acid transport polypeptide by increasing its expression; the genetic modification is the addition of a gene encoding an organic acid transport polypeptide; the genetic modification is a genetic modification of a gene encoding an organic acid transport polypeptide that increases transcription or translation of the gene or a genetic modification that alters the coding sequence of a gene encoding an organic acid transport polypeptide the organic acid transport polypeptide is heterologous to the yeast; the organic acid transport polypeptide has an amino acid sequence identical to that of an organic acid transport polypeptidepolypeptide from an organism of the Schizosaccharomyces genus; the organic acid transport polypeptide has an amino acid sequence identical to that of a Schizosaccharomyces pombe MAE1 polypeptide; the organic acid transport polypeptide has at least 75% identity to SEQ ID NO:43 (Sp MAE1); the organic acid transport polypeptide has at least 95% identity to SEQ ID NO:43 (Sp MAE1); the organic acid transport polypeptide has an amino acid sequence identical to that of a Brassica napus ALMT1 polypeptide; the organic acid transport polypeptide has at least 75% identity to SEQ ID NO:45 (Brassica napus ALMT1); the organic acid transport polypeptide has at least 95% identity to SEQ ID NO:45 (Brassica napus ALMT1); the organic acid transport polypeptide has an amino acid sequence identical to that of a Triticum secale ALMT1 polypeptide; the organic acid transport polypeptide has at least 75% identity to SEQ ID NO:47 (Triticum secale ALMT1); the organic acid transport polypeptide has at least 95% identity to SEQ ID NO:47 (Triticum secale ALMT1); the organic acid transport polypeptide has an amino acid sequence identical to that of K. lactis Jen1; the organic acid transport polypeptide has at least 75% identity to SEQ ID NO:25 (K. lactis Jen1); the organic acid transport polypeptide has at least 95% identity to SEQ ID NO:25 (K. lactis Jen1); the organic acid transport polypeptide has an amino acid sequence identical to that of S. cerevisiae Jen1; the organic acid transport polypeptide has at least 75% identity to SEQ ID NO:29 (S. cerevisiae Jen1); the organic acid transport polypeptide has at least 95% identity to SEQ ID NO:29 (S. cerevisiae Jen1); the organic acid transport polypeptide has an amino acid sequence identical to that of K. lactis JEN2; the organic acid transport polypeptide has at least 75% identity to SEQ ID NO:27 (K. lactis JEN2); the organic acid transport polypeptide has at least 95% identity to SEQ ID NO:27 (K. lactis JEN2); the organic acid transport polypeptide has an amino acid sequence identical to that of M. musculus NaDC1; the organic acid transport polypeptide has at least 75% identity to SEQ ID NO:31 (M. musculus NaDC1); the organic acid transport polypeptide has at least 95% identity to SEQ ID NO:31 (M. musculus NaDC1); the organic acid transport polypeptide has an amino acid sequence identical to that of Streptococcus bovis malP; the organic acid transport polypeptide has at least 75% identity to SEQ ID NO:33 (Streptococcus bovis malP); the organic acid transport polypeptide has at least 95% identity to SEQ ID NO:33 (Streptococcus bovis malP); the organic acid transport polypeptide has an amino acid sequence identical to that of A. thaliana AttDT; the organic acid transport polypeptide has at least 75% identity to SEQ ID NO:35 (A. thaliana AttDT the organic acid transport polypeptide has at least 95% identity to SEQ ID NO:35 (A. thaliana AttDT); the organic acid transport polypeptide has an amino acid sequence identical to that of R. norvegicus NaDC3; the organic acid transport polypeptide has at least 75% identity to SEQ ID NO:37 (R. norvegicus NaDC3); the organic acid transport polypeptide has at least 95% identity to SEQ ID NO:37 (R. norvegicus NaDC3); the organic acid transport polypeptide has an amino acid sequence identical to that of H. sapiens Mct1; the organic acid transport polypeptide has at least 75% identity to SEQ ID NO:39 (H. sapiens Mct1); the organic acid transport polypeptide has at least 95% identity to SEQ ID NO:39 (H. sapiens Mct1); the organic acid transport polypeptide has an amino acid sequence identical to that of H. sapiens Mct2; the organic acid transport polypeptide has at least 75% identity to SEQ ID NO:41 (H. sapiens Mct2); the organic acid transport polypeptide has at least 95% identity to SEQ ID NO:41 (H. sapiens Mct2); the organic acid transport polypeptide has an amino acid sequence identical to that of a an organic acid transport polypeptide in FIG. 24; the organic acid transport polypeptide has at least 75% identity to an organic acid transport polypeptide in FIG. 24; and the organic acid transport polypeptide has at least 95% identity to an organic acid transport polypeptide in FIG. 24; the organic acid transport polypeptide has at least 75%, 80%, 85%, 90%, 95%, 98% or 99% identity to Aspergillus oryzae organic acid transport polypeptide (SEQ ID NO:______).

Also described is a modified yeast having at least two modifications as compared with a parental yeast, the at least two modifications including: a first modification that reduces PDC polypeptide activity; and at least one additional modification selected from the group consisting of a modification that increases pyruvate carboxylase (PYC) polypeptide activity, a modification that increases phosphoenolpyruvate carboxylase polypeptide activity (PPC activity), a modification that increases malate dehydrogenase (MDH) polypeptide activity, and modification that increases organic acid transport (MAE) polypeptide activity. In various cases: the modified yeast has at least two of the additional modifications; the modified yeast has at least three of the additional modifications; the modified yeast has all of the additional modifications; at least one of the additional modifications comprises a genetic modification; at least one of the genetic modifications comprises introducing into a yeast cell a gene encoding the relevant polypeptide; the introduced gene has an amino acid sequence identical, at least 95% identical, or at least 75% identical to that found in a source organism selected from the group consisting of Saccharomyces cerevisiae, Yarrowia lipolytica, Aspergillus niger, Nocardia sp. JS614, Methanothermobacter thermautotrophicus str. Delta H, Actinobacillus succinogenes, Actinobacillus pleuropneumoniae, Escherichia coli, Erwinia carotovora, Erwinia chrysanthemi, (Thermo)synechococcus vulcanus, Streptococcus bovis, Corynebacterium glutamicum, Arabidopsis thaliana, Brassica napes, Triticum secale, Rattus norvegicus, Mus musculus or Homo sapiens; the source organism is selected from Saccharomyces cerevisiae, Schizosaccharomyces pombe, and Escherichia coli; A. oryzae each introduced gene is from the same source; and different introduced genes are from different sources.

Also described is a method of producing malic acid, comprising: culturing a modified yeast of described herein under conditions that achieve malic acid production. In various cases: the method further comprises: a step of isolating malic acid. In some cases: the step of culturing under conditions that achieve malic acid production comprises culturing at a pH within the range of 1.5 to 7; the pH is lower than 5.0; the pH is lower than 4.5; the pH is lower than 4.0; the pH is lower than 3.5; the pH is lower than 3.0; the pH is lower than 2.5; the pH is lower than 2.0; the step of culturing under conditions that achieve malic acid production comprises culturing under conditions and for a time sufficient for malic acid to accumulate to a level within the range of 10 to 200 g/L (greater than 30 g/L; greater than 50 g/L; greater than 75 g/L; greater than 100 g/L; greater than 125 g/L; or greater than 150 g/L); the step of culturing under conditions that achieve malic acid production comprises culturing under conditions and for a time sufficient for malic acid to accumulate to a level within a range of about 0.3 moles of malic acid per mole of substrate to about 1.75 moles of malic acid per mole of substrate; malic acid accumulates to greater than about 0.3 moles of malic acid per mole of substrate; malic acid accumulates to greater than about 0.5 moles of malic acid per mole of substrate malic acid accumulates to greater than about 0.75 moles of malic acid per mole of substrate; malic acid accumulates to greater than about 1.0 moles of malic acid per mole of substrate; malic acid accumulates to greater than about 1.25 moles of malic acid per mole of substrate; malic acid accumulates to greater than about 1.5 moles of malic acid per mole of substrate; malic acid accumulates to greater than about 1.75 moles of malic acid per mole of substrate; the substrate is glucose; the step of culturing under conditions that achieve malic acid production comprises culturing in a medium comprising a carbon source; the carbon source is one or more carbon sources selected from the group consisting of glucose, glycerol, sucrose, fructose, maltose, lactose, galactose, hydrolyzed starch, corn syrup, high fructose corn syrup, and hydrolyzed lignocelluloses; the medium further comprises a carbon dioxide source; and the carbon dioxide source comprises calcium carbonate or carbon dioxide gas.

Also described is a method of producing succinic acid, comprising culturing a modified yeast described herein under conditions that achieve succinic acid production. In various cases: the method further comprises: a step of isolating produced succinic acid; the step of culturing comprises culturing in a medium comprising a carbon source; the carbon source is one or more carbon sources selected from the group consisting of glucose, glycerol, sucrose, fructose, maltose, lactose, galactose, hydrolyzed starch, corn syrup, high fructose corn syrup, and hydrolyzed lignocelluloses; the carbon source is glucose; the medium further comprises a carbon dioxide source; and the carbon dioxide source comprises calcium carbonate or carbon dioxide gas.

Also described is a method of preparing a food or feed additive containing malic acid or succinic acid, the method comprising steps of a) cultivating a modified yeast described herein under conditions that allow production of malic acid or succinic acid; b) isolating one or both of the malic acid and succinic acid; and c) combining one or both of the isolated malic acid or succinic acid with one or more other food or feed additive components; and the product of this method.

Also described is a method of preparing a cosmetic containing malic acid or succinic acid, the method comprising steps of a) cultivating a modified yeast described herein under conditions that allow production of the malic acid or succinic acid; b) isolating one or both of the malic acid and succinic acid; and c) combining one or both of the isolated malic acid or succinic acid with one or more cosmetic components; and the product of this method.

Also described is a method of preparing an industrial chemical containing malic acid or succinic acid, the method comprising steps of: a) cultivating a modified yeast described herein under conditions that allow production of the malic acid or succinic acid; b) isolating one or both of the malic acid and succinic acid; and c) combining one or more of the isolated malic acid or succinic acid with one or more industrial chemical components; and the product of this method.

Also described is a method of preparing a biodegradable polymer containing malic acid or succinic acid, the method comprising steps of a) cultivating a modified yeast described herein under conditions that allow production of the malic acid or succinic acid; b)

isolating one or more of the malic acid and succinic acid; and c) combining one or more of the isolated malic acid or succinic acid with one or more biodegradable polymer components; and the product of this method.

Accumulation: As used herein, “accumulation” of malic acid above background levels refers to accumulation to detectable levels. In some embodiments, “accumulation” refers to accumulation above a pre-determined level (e.g., above a level achieved under otherwise identical conditions with a yeast that has not been modified as described herein). In other embodiments, “accumulation” refers to titer of an organic acid, i.e. grams per liter of one or more organic acids in the broth of a cultured fungus. Any available assay, including those explicitly set forth herein, may be used to detect and/or quantify malic acid and/or succinic acid accumulation.

Amplification: The term “amplification” refers to increasing the number of copies of a desired nucleic acid molecule. Typically, amplification results in an increased level of activity of an enzyme, and/or to an increased level of activity in a desirable location (e.g., in the cytosol).

Codon: As is known in the art, the term “codon” refers to a sequence of three nucleotides that specify a particular amino acid.

DNA ligase: The term “DNA ligase” refers to an enzyme that covalently joins two pieces of double-stranded DNA.

Electroporation: The term “electroporation” refers to a method of introducing foreign DNA into cells that uses a brief, high voltage DC charge to permeabilize the host cells, causing them to take up extra-chromosomal DNA.

Endonuclease: The term “endonuclease” refers to an enzyme that hydrolyzes double stranded DNA at internal locations.

Expression: The term “expression” refers to the production of a gene product (i.e., RNA or protein). For example, “expression” includes transcription of a gene to produce a corresponding mRNA, and translation of such an mRNA to produce the corresponding peptide, polypeptide, or protein.

Functionally linked: The phrase “functionally linked” or “operably linked” refers to a promoter or promoter region and a coding or structural sequence in such an orientation and distance that transcription of the coding or structural sequence may be directed by the promoter or promoter region.

Functionally transformed: As used herein, the term “functionally transformed” refers to a yeast cell that has been caused to express one or more polypeptides (e.g., pyruvate carboxylase polypeptide, phosphoenolpyruvate carboxylase polypeptide, malate dehydrogenase polypeptide, and/or organic acid transport polypeptide) as described herein, such that the expressed polypeptide is functional and is active at a level higher than is observed with an otherwise identical yeast cell that has not been so transformed. In many embodiments, functional transformation involves introduction of a nucleic acid encoding the polypeptide(s) such that the polypeptide(s) is/are produced in an active form and/or appropriate location. Alternatively or additionally, in some embodiments, functional transformation involves introduction of a nucleic acid that regulates expression of such an encoding nucleic acid.

Gene: The term “gene”, as used herein, generally refers to a nucleic acid encoding a polypeptide, optionally including certain regulatory elements that may affect expression of one or more gene products (i.e., RNA or protein). A gene may be in chromosomal DNA, plasmid DNA, cDNA, synthetic DNA, or other DNA that encodes a peptide, polypeptide, protein, or RNA molecule, and may include regions flanking the coding sequence involved in the regulation of expression.

Genome: The term “genome” encompasses both the chromosomes and plasmids within a host cell. For example, encoding nucleic acids of the present disclosure that are introduced into host cells can be part of the genome whether they are chromosomally integrated or plasmid-localized.

Heterologous: The term “heterologous”, means from a source other than the host cell. For example, “heterologous”genetic material or polypeptides are those that do not naturally occur in the organism in which they are present and/or being expressed. It will be understood that, in general, when heterologous genetic material or polypeptide is selected for introduction into and/or expression by a host cell, the particular source organism from which the heterologous genetic material or polypeptide may be selected is not essential to the practice of the present disclosure. Relevant considerations may include, for example, how closely related the potential source and host organisms are in evolution, or how related the source organism is with other source organisms from which sequences of other relevant polypeptides have been selected. Where a plurality of different heterologous polypeptides and/or genetic sequences are to be introduced into and/or expressed by a host cell, different polypeptides or sequences may be from different source organisms, or from the same source organism. To give but one example, in some cases, individual polypeptides may represent individual subunits of a complex protein activity and/or may be required to work in concert with other polypeptides in order to achieve the goals of the present disclosure. In some embodiments, it will often be desirable for such polypeptides to be from the same source organism, and/or to be sufficiently related to function appropriately when expressed together in a host cell. In some embodiments, such polypeptides may be from different, even unrelated source organisms. It will further be understood that, where a heterologous polypeptide is to be expressed in a host cell, it will often be desirable to utilize nucleic acid sequences encoding the polypeptide that have been adjusted to accommodate codon:preferences of the host cell and/or to link the encoding sequences with regulatory elements active in the host cell.

Homologous: The term “homologous”, as used herein, means from the same source as the host cell. For example, as used here to refer to genetic material or to polypeptides, the term “homologous” refers to genetic material or polypeptides that naturally occurs in the organism in which it is present and/or being expressed, although optionally at different activity levels and/or in different amounts.

Host cell: As used herein, the “host cell” is a yeast cell that is manipulated according to the present disclosure to increase production of malic acid as described herein. A “modified host cell”, as used herein, is any host cell which has been modified, engineered, or manipulated in accordance with the present disclosure as compared with a parental cell. In some embodiments, the modified host cell has at least one maleogenic modification(s). In some embodiments, the parental cell is a naturally occurring parental cell.

Hybridization: “Hybridization” refers to'the ability of a strand of nucleic acid to join with a complementary strand via base pairing. Hybridization occurs when complementary sequences in the two nucleic acid strands bind to one another.

Isolated: The term “isolated”, as used herein, means that the isolated entity has been separated from at least one component with which it was previously associated. When most other components have been removed, the isolated entity is “purified” or “concentrated”. Isolation and/or purification and/or concentration may be performed using any techniques known in the art including, for example, fractionation, extraction, precipitation, or other separation.

Medium: As is known in the art, the term “medium” refers to the chemical environment of the yeast comprising any component required for the growth of the yeast or the recombinant yeast and one or more precursors for the production of malic acid and/or succinic acid. Components for growth of the yeast and precursors for the production of malic acid and/or succinic acid may or may be not identical.

Modified: The term “modified”, as used herein, refers to a host organism that has been modified to increase production of malic acid and/or succinic acid, as compared with an otherwise identical host organism that has not been so modified. In principle, such “modification” in accordance with the present disclosure may comprise any chemical, physiological, genetic, or other modification that appropriately alters production of malic acid and/or succinic acid in a host organism as compared with such production in an otherwise identical organism not subject to the same modification. In most embodiments, however, the modification will comprise a genetic modification. In certain embodiments, as described herein, the modification comprises introducing into a host cell, and particularly into a host cell that is reduced or negative for pyruvate decarboxylase (PDC) activity. In some embodiments, a modification comprises at least one chemical, physiological, genetic, or other modification; in other embodiments, a modification comprises more than one chemical, physiological, genetic, or other modification. In certain aspects where more than one modification is utilized, such modifications can comprise any combination of chemical, physiological, genetic, or other modification (e.g., one or more genetic, chemical and/or physiological modification(s)). Genetic modifications that increase the activity of a polypeptide include, but are not limited to: introducing one or more copies of a gene encoding the polypeptide (which may differ from any gene already present in the host cell encoding a polypeptide having the same activity); altering a gene present in the cell to increase transcription or translation of the gene (e.g., altering, adding additional sequence to, deleting sequence from, replacement of one or more nucleotides, or swapping for example, a promoter, regulatory or other sequence); and altering the sequence (e.g. coding or non-coding) of a gene encoding the polypeptide to increase activity (e.g., by increasing catalytic activity, reducing feedback inhibition, targeting a specific subcellular location, increasing mRNA stability, increasing protein stability). Genetic modifications that decrease activity of a polypeptide include, but are not limited to: deleting all or a portion of a gene encoding the polypeptide; inserting a nucleic acid sequence that disrupts a gene encoding the polypeptide; altering a gene present in the cell to decrease transcription or translation of the gene or stability of the mRNA or polypeptide encoded by the gene (for example, by altering, adding additional sequence to, deleting sequence from, replacement of one or more nucleotides, or swapping for example, a promoter, regulatory or other sequence).

Open reading frame: As is known in the art, the term “open reading frame (ORF)” refers to a region of DNA or RNA encoding a peptide, polypeptide, or protein.

PDC-reduced: As used herein, the term “PDC-reduced” refers to a yeast cell containing a modification, e.g., a genetic modification, that reduces pyruvate decarboxylase activity as compared with an otherwise identical yeast that is not modified. Pyruvate decarboxylase activity can be provided by any thiamin diphosphate-dependent enzyme that catalyses the decarboxylation of pyruvic acid to acetaldehyde and carbon dioxide (EC 4.1.1.1). The reduction in activity can arise from a reduction in the level of one or more pyruvate decarboxylase polypeptides relative to an unmodified yeast cell or it can result from one or more modifications, e.g, genetic modifications that reduce the activity (e.g, catalytic activity) of the one or more pyruvate decarboxylase polypeptides relative to an unmodified cell without substantially altering the level of the one or more pyruvate decarboxylase polypeptides. The reduction in activity can also arise from a combination of lowered polypeptide levels and lowered activity. In some embodiments, a PDC-reduced yeast cell has reduced activity of one or more pyruvate decarboxylase polypeptides relative to the unmodified yeast cell. In certain embodiments thereof the pyruvate decarboxylase polypeptide is chosen from one or more of Pdc1, Pdc2, Pdc5, Pdc6 polypeptides including any of the pyruvate decarboxylase and Pdc2 polypeptides in FIG. 20.

In some embodiments a PDC-reduced cell has reduced or substantially eliminated Pdc1 polypeptide activity. In certain embodiments, the PDC-reduced cell further comprises reduced or substantially eliminated Pdc2, Pdc5, and/or Pdc6 polypeptide activity. In some embodiments a PDC-reduced cell has reduced or substantially eliminated Pdc2 polypeptide activity. In certain embodiments thereof, the PDC-reduced cell further comprises reduced or substantially eliminated Pdc1, Pdc5, and/or Pdc6 polypeptide activity. In some embodiments a PDC-reduced cell has reduced or substantially eliminated Pdc5 polypeptide activity. In certain embodiments thereof, the PDC-reduced cell further comprises reduced and/or substantially eliminated Pdc1, Pdc2, and/or Pdc6 polypeptide activity. In some embodiments a PDC-reduced cell has reduced or substantially eliminated Pdc6 polypeptide activity. In certain embodiments thereof, the PDC-reduced cell further comprises reduced and/or substantially eliminated Pdc1, Pdc2, and/or Pdc5 polypeptide activity. In some embodiments a PDC-reduced cell has reduced and/or substantially eliminated Pdc1 and Pdc5 polypeptide activity. In some embodiments a PDC-reduced cell has reduced and/or substantially eliminated Pdc1 and Pdc6 polypeptide activity. In some embodiments a PDC-reduced cell has reduced and/or substantially eliminated Pdc5 and Pdc6 polypeptide activity. In some embodiments a PDC-reduced cell has reduced and/or substantially eliminated Pdc1, Pdc5 and Pdc6 polypeptide activity. In some embodiments, a PDC-reduced cell has 3-fold, 5-fold, 10-fold, 50-fold less pyruvate decarboxylase activity as compared with an otherwise identical parental cell not containing the modification. In some embodiments, a PDC-reduced cell has pyruvate decarboxylase activity below at least about 0.075 micromol/min mg protein⁻¹, at least about 0.045 micromol/min mg protein⁻¹, at least about 0.025 micromol/min mg protein⁻¹; in some embodiments, a PDC-reduced cell has pyruvate decarboxylase activity below about 0.005 micromol/min mg protein⁻¹ when using the methods described by van Maris et. al. (Overproduction of Threonine Aldolase Circumvents the Biosynthetic Role of Pyruvate Decarboxylase in Glucose-grown Saccharomyces cerevisiae. Appl. Environ. Microbiol. 69:2094-2099, 2003). In some embodiments, a PDC-reduced cell has no detectable pyruvate decarboxylase activity. In some embodiments, a cell with no detectable pyruvate decarboxylase activity is referred tows “PDC-negative”. In some embodiments a PDC-negative cell lacks Pdc1, Pdc5 and Pdc6 polypeptide activity. In some embodiments a PDC-negative cell has pyruvate decarboxylase activity below about 0.005 micromol/min mg protein⁻¹.

Plasmid: As is known in the art, the term “plasmid” refers to a circular, extra chromosomal, replicatable piece of DNA.

Polymerase chain reaction: As is known in the art, the term “polymerase chain reaction (PCR)” refers to an enzymatic technique to create multiple copies of one sequence of nucleic acid. Copies of DNA sequence are prepared by shuttling a DNA polymerase between two amplimers. The basis of this amplification method is multiple cycles of temperature changes to denature, then re-anneal amplimers, followed by extension to synthesize new DNA strands in the region located between the flanking amplimers.

Polypeptide: The term “polypeptide”, as used herein, generally has its art-recognized meaning of a polymer of at least three amino acids. However, the term is also used to refer to specific functional classes of polypeptides, such as, for example, pyruvate decarboxylase (PDC), pyruvate carboxylase (PYC), phosphoenolpyruvate carboxylase (PPC), malate dehydrogenase (MDH) polypeptides, and/or organic acid transport (MAE) polypeptides, etc. For each such class, the present specification provides several examples of known sequences of such polypeptides. Those of ordinary skill in the art will appreciate, however, that the term “polypeptide” is intended to be sufficiently general as to encompass not only polypeptides having the complete sequence recited herein (or in a reference or database specifically mentioned herein), but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in, one or more highly conserved regions usually encompassing at least 3-4 and often up to 20 or more amino acids, with another polypeptide of the same class, is encompassed within the relevant term “polypeptide” as used herein. Other regions of similarity and/or identity can be determined by those of ordinary skill in the art by analysis of the sequences of various polypeptides presented in FIGS. 18, 20-24 and 26 herein. For example, using various well-known algorithms, one of ordinary skill in the art can align the amino acid sequences of two or more polypetides having the same enzymatic activity and thereby identify more conserved and less conserved regions of the polypeptides. Hidden Markov Models and other analytical tools can also be used to identify important functional domains.

Promoter: As is known in the art, the term “promoter” or “promoter region” refers to a DNA sequence, usually found upstream (5′) to a coding sequence, that controls expression of the coding sequence by controlling production of messenger RNA (mRNA) by providing the recognition site for RNA polymerase and/or other factors necessary for start of transcription at the correct site.

Recombinant: A “recombinant” yeast, as that term is used herein, is a yeast that has been modified to increase its, production of malic acid and/or succinic acid, through modification, for example, genetic modification. For example, a “recombinant cell” can be a cell that contains a nucleic acid sequence not naturally occurring in the cell, or an additional copy or copies of an endogenous nucleic acid sequence, wherein the nucleic acid sequence is introduced into the cell or an ancestor thereof by human action. A recombinant cell includes, but is not limited to: a cell which has been genetically modified by deletion of all or a portion of a gene, a cell that has had a mutation introduced into a gene, and a cell that has had a nucleic acid sequence inserted either to add a functional gene or disrupt a functional gene. A “recombinant vector” or “recombinant DNA or RNA construct” refers to any nucleic acid molecule generated by the hand of man. For example, a recombinant construct may be a vector such as a plasmid, cosmid, virus, autonomously replicating sequence, phage, or linear or circular single-stranded or double-stranded DNA or RNA molecule. A recombinant nucleic acid may be derived from any source and/or capable of genomic integration or autonomous replication where it includes two or more sequences that have been linked together by the hand of man. Recombinant constructs may, for example, be capable of introducing a 5′ regulatory sequence or promoter region and a DNA sequence for a selected gene product into a cell in such a manner that the DNA sequence is transcribed into a functional mRNA, which may or may not be translated and therefore expressed.

Restriction enzyme: As is known in the art, the term “restriction enzyme” refers to an enzyme that recognizes a specific sequence of nucleotides in double stranded DNA and cleaves both strands; also called a restriction endonuclease. Cleavage typically occurs within the restriction site or close to it.

Selectable: The term “selectable” is used to refer to a marker whose expression confers a phenotype facilitating identification, and specifically facilitating survival, of cells containing the marker. Selectable markers include those, which confer resistance to toxic chemicals (e.g. ampicillin, kanamycin) or complement a nutritional deficiency (e.g. uracil, histidine, leucine).

Screenable: The term “screenable” is used to refer to a marker whose expression confers a phenotype facilitating identification, optionally without facilitating survival, of cells containing the marker. In many embodiments, a screenable marker imparts a visually or otherwise distinguishing characteristic (e.g. color changes, fluorescence).

Source organism: The term “source organism”, as used herein, refers to the organism in which a particular polypeptide or genetic sequence can be found in nature. Thus, for example, if one or more homologous or heterologous polypeptides or genetic sequences is/are being expressed in a host organism, the organism in which the polypeptides or sequences are expressed in nature (and/or from which their genes were originally cloned) is referred to as the “source organism”. Where multiple homologous or heterologous polypeptides and/or genetic sequences are being expressed in a host organism, one or more source organism(s) may be utilized for independent selection of each of the heterologous polypeptide(s) or genetic sequences. It will be appreciated that any and all organisms that naturally contain relevant polypeptide or genetic sequences maybe used as source organisms in accordance with the present disclosure. Representative source organisms include, for example, animal, mammalian, insect, plant, fungal, yeast, algal, bacterial, archaebacterial, cyanobacterial, and protozoal source organisms. For example a source organism may be a fungus, including yeasts, of the genus Saccharomyces, Yarrowia, Aspergillus, Schizosaccharomyces, or Kluyveromyces. In certain embodiments, the source organism may be of the species Saccharomyces cerevisiae, Yarrowia lipolytica, Aspergillus niger, Aspergillus oryzae, Schizosaccharomyces pombe, or Kluyveromyces lactis. For example a source organism may be a bacterium, including an archaebacterium, of the genus Nocardia, Methanothermobacter, Actinobacillus, Escherichia, Erwinia, (Thermo)synechococcus, Streptococcus or Corynebacterium. In certain embodiments, the source organism may be of the species Nocardia sp. JS614, Methanothermobacter thermautotrophicus str.Delta H, Actinobacillus succinogenes, Actinobacillus pleuropneumoniae, Escherichia coli, Erwinia carotovora, Erwinia chrysanthemi, (Thermo)synechococcus vulcanus, Streptococcus bovis or Corynebacterium glutamicum. For example a source organism may be a plant of the genus Arabidopsis, Brassica or Triticum. In certain embodiments, the source organism may be of the species Arabidopsis thaliana, Brassica napus or Triticum secale. For example a source organism may be a mammal of the genus Rattus, Mus or Homo. In certain embodiments, the source organism may be of the species Rattus norvegicus, Mus musculus or Homo sapiens. As used herein, polypeptide (or nucleic acid) is considered to be of a particular source organism if it has an amino acid (or nucleotide) sequence identical or substantially identical to that of of a polypeptide found in that organism in nature.

Transcription: As is known in the art, the term “transcription” refers to the process of producing an RNA copy from a DNA template.

Transformation: The term “transformation”, as used herein, typically refers to a process of introducing a nucleic acid molecule into a host cell. Transformation typically achieves a genetic modification of the cell. The introduced nucleic acid may integrate into a chromosome of a cell, or may replicate autonomously. A cell that has undergone transformation, or a descendant of such a cell, is “transformed” and is a “recombinant” cell. Recombinant cells are modified cells as described herein. If the nucleic acid that is introduced into the cell comprises a coding region encoding a desired protein, and the desired protein is produced in the transformed yeast and is substantially functional, such a transformed yeast is “functionally transformed.” Cells herein may be transformed with, for example, one or more of a vector, a plasmid or a linear piece (e.g., a linear piece of DNA created by linearizing a vector or a linear piece of DNA created by PCR amplification) of DNA to become functionally transformed.

Translation: As is known in the art, the term “translation” refers to the production of protein from messenger RNA.

Yield: The term “yield”, as used herein, refers to the amount of desired product (e.g. malic acid and/or succinic acid) produced (molar or weight/volume) divided by the amount of carbon source (e.g. dextrose) consumed (molar or weight/volume), multiplied by 100.

Unit: The term “unit”, when used to refer to an amount of an enzyme, refers to the enzymatic activity and indicates the amount of micromoles of substrate converted per mg of total cell proteins per minute.

Vector: The term “vector” as used herein refers to a DNA or RNA molecule (such as a plasmid, cosmid, bacteriophage, yeast artificial chromosome, or virus, among others) that carries nucleic acid sequences into a host cell. A vector for use in accordance with the present disclosure can be a plasmid, a cosmid, or a yeast artificial chromosome, among others known in the art to be appropriate for use in yeast. The vector may be linear or circular. The vector or a portion of it can be inserted into the genome'of the host cell. A vector can comprise an origin of replication, which allows the vector to be passed on to progeny cells of a yeast comprising the vector. Alternatively, if integration of the vector into the yeast genome is desired, the vector can comprise sequences homologous to sequences found in the yeast genome, and can also comprise coding regions that can facilitate integration. The homologous sequences found in the yeast genome may be endogenous to yeast. Alternatively, the homologous sequences may be sequences that are artificially derived or are from another organism that are inserted into the yeast genome prior to integration of the vector. To determine which yeast cells are transformed, the vector can comprise a detectable (i.e., screenable or selectable marker). A vector may comprise any of a variety of other genetic elements, such as restriction endonuclease sites and others typically found in vectors.

### BRIEF DESCRIPTION OF THE DRAWING

FIG. 1 shows glucose and pyruvate concentrations as a function of culture time as described in Example 1.

FIG. 2 shows malate, glycerol, and succinate concentrations as a function of culture time as described in Example 1.

FIG. 3 is a map of plasmid p426GPDMDH3, as described in Example 1.

FIG. 4 is a map of plasmid pRS2, as described in Example 1.

FIG. 5 is a map of plasmid pRS2ΔMDH3, as described in Example 1.

FIG. 6 is a map of plamid YEplac112 SpMAE1, as described in Example 1.

FIG. 7 shows the biomass, the consumption of glucose, and the production of pyruvate in Batch A, Example 2.

FIG. 8 shows the production of malate, glycerol, and succinate in Batch A, Example 2.

FIG. 9 shows the biomass, the consumption of glucose, and the production of pyruvate in Batch B, Example 2.

FIG. 10 shows the production of malate, glycerol, and succinate in Batch B, Example 2.

FIG. 11 shows the biomass, the consumption of glucose, and the production of pyruvate in Batch C, Example 2.

FIG. 12 shows the production of malate, glycerol, and succinate in Batch C, Example 2.

FIG. 13 shows the effect of various inhibitors on wild-type E. coli PPC activity.

FIG. 14 shows the effect of various inhibitors on mutant E. coli PPC activity.

FIG. 15 shows fermentation results from PDC6/pdc6 and pdc6/pdc6 diploid strains.

FIG. 16 shows fermentation results from PDC6 and pdc6 haploid strains.

FIG. 17 shows fermentation results from strains expressing a Mdh2 (P2S) variant protein.

FIG. 18 is a table with amino acid sequences of exemplary proteins for organic acid production in fungal cells.

FIG. 19 is a table with nucleotide sequences encoding exemplary proteins for organic acid production in fungal cells.

FIG. 20 is a table of exemplary pyruvate decarboxylase polypeptides for organic acid production in fungal cells.

FIG. 21 is a table of exemplary phosphoenolpyruvate carboxylase polypeptides for organic acid production in fungal cells.

FIG. 22 is a table of exemplary pyruvate carboxylase polypeptides for organic acid production in fungal cells.

FIG. 23 is a table of exemplary malate dehydrogenase polypeptides for organic acid production in fungal cells.

FIG. 29 is a table of exemplary organic acid transport polypeptides for organic acid production in fungal cells.

FIGS. 25a-e depict malic acid and succinic acid and representative pathways for their production.

FIG. 26 is a table of exemplary organic acid transporter polypeptides for organic acid production in fungal cells.

### DETAILED DESCRIPTION

In certain embodiments, the present disclosure relates to a modified (e.g., recombinant) yeast, wherein the yeast has reduced pyruvate decarboxylase enzyme (PDC) activity (i.e., is PDC-reduced or PDC-negative) and is functionally transformed to increase the activity of either a pyruvate carboxylase (PYC) polypeptide, a phosphoenolpyruvate carboxylase (PPC) polypeptide, a malate dehydrogenase (MDH) polypeptide, and/or an organic acid transport (MAE) polypeptide.

In some embodiments, the recombinant yeast is functionally transformed to increase the activity of a PYC polypeptide or a PPC polypeptide, together with modifications to increase the activities of a MDH polypeptide and a MAE polypeptide.

In some embodiments, the modified (e.g., recombinant) PDC-reduced yeast is functionally transformed to increase the activity of a PYC polypeptide that is active in the cytosol (e.g., by a genetic modification that increases the level or fraction of PYC polypeptide present in the cell compared to an otherwise identical cell lacking the genetic modification). In some embodiments, the recombinant PDC-reduced yeast is functionally transformed to increase the activity of a PPC polypeptide that is less sensitive to inhibition by one more of malate, aspartate, and oxaloacetate (e.g., there is a modification such as a genetic modification in PPC that reduces inhibition compared to an otherwise identical cell). In some embodiments, the recombinant PDC-reduced yeast is functionally transformed to increase the activity of a MDH polypeptide that exhibits increased activity in the cytosol and/or is less sensitive to inactivation in the presence of glucose. Any yeast known in the art for use in industrial processes can be used according to the present disclosure as a matter of routine experimentation by the skilled artisan having the benefit of the present disclosure.

For example, the yeast to be modified (e.g., transformed) can be selected from any known genus and species of yeast. Yeasts are described by N. J. W. Kreger-van Rij, “The Yeasts,” Vol. 1 of Biology of Yeasts, Ch. 2, A. H. Rose and J. S. Harrison, EdS. Academic Press, London, 1987. In one embodiment, the yeast genus can be Saccharomyces, Zygosaccharomyces, Candida, Hansenula, Kluyveromyces, Debaromyces, Nadsonia, Lipomyces, Torulopsis, Kloeckera, Pichia, Schizosaccharomyces, Trigonopsis, Brettanomyces, Cryptococcus, Trichosporon, Aureobasidium, Lipomyces, Phaffia, Rhodotorula, Yarrowia, or Schwanniomyces, among others. In a further embodiment, the yeast can be a Saccharomyces, Zygosaccharomyces, Yarrowia, Kluyveromyces or Pichia spp. In yet a further embodiment, the yeast can be Saccharomyces cerevisiae, Saccharomyces cerevisiae var bayanus (e.g. Lalvin DV10), Saccharomyces boulardii, Zygosaccharomyces bailii, Kluyveromyces lactis, and Yarrowia lipolytica. Saccharomyces cerevisiae is a commonly used yeast in industrial processes, but the disclosure is not limited thereto. Other yeast species useful in the present disclosure include but are not limited to Hansenula anomala, Schizosaccharomyces pombe, Candida sphaerica, and Schizosaccharomyces malidevorans.

A “recombinant” yeast is a yeast that has been modified (e.g, genetically modified by the sequence alteration, addition or deletion or all or part of a gene) to increase its production of malic acid and/or succinic acid. Such a yeast is said to have a “maleogenic modification” or a “succinogenic modification”.

In some embodiments of the disclosure, a recombinant yeast contains a nucleic acid sequence not naturally occurring in the yeast or an additional copy or copies of an endogenous nucleic acid sequence, wherein the nucleic acid sequence is introduced into the yeast or an ancestor cell thereof by human action. Recombinant DNA techniques are well-known, such as in Sambrook et al., Molecular Genetics: A Laboratory Manual, Cold Spring Harbor Laboratory Press, which provides further information regarding various techniques known in the art and discussed herein. In some embodiments, such introduced sequences may comprise coding sequences; in some embodiments, such introduced sequences may comprise regulatory sequences.

In some embodiments, a recombinant yeast is constructed by introduction of part or all of the coding region of a homologous or heterologous gene into a host yeast cell. Such a coding region may be isolated from a source organism that possesses the gene. This source organism can be a bacterium, a prokaryote, a eukaryote, a microorganism, a fungus, a plant, or an animal.

Genetic material comprising coding and/or regulatory sequences of interest can be extracted from cells of a source organism by any known technique and/or can be isolated by any appropriate technique. In one known technique, such material is isolated by, first, preparing a genomic DNA library or a cDNA library, and second, identifying desired sequences in a genomic DNA library or cDNA library, such as by probing the library with a labeled nucleotide probe selected to be or presumed to be at least partially homologous with the desired sequences, determining whether expression or activity of the desired sequences imparts a detectable phenotype to a library microorganism comprising them, and/or amplifying the desired sequence by PCR. Other known techniques for isolating or otherwise preparing desired sequences (including, for example, chemical synthesis) can also be used.

“PDC-reduced” is used herein to describe a yeast with reduced PDC activity. In some embodiments, a yeast has pyruvate decarboxylase activity below about 0.005 micromol/min mg protein⁻¹. Such a yeast may be referred to herein as having “no PDC activity”, or as being “PDC-negative.” The terms PDC-reduced and PDC-negative are further discussed above.

A yeast which is PDC-reduced can be isolated or engineered by any appropriate technique. For example, a large starting population of genetically-diverse yeast may contain natural mutants which arc PDC-reduced (e.g., PDC-negative). A starting population can be subjected to mutagenesis or chemostat-based selection. A typical PDC-positive yeast strain comprises (A) at least one PDC structural gene that is capable of being expressed in the yeast strain; (B) at least one PDC regulatory gene that is capable of being expressed in the yeast strain; (C) a promoter of the PDC structural, gene; and (D) a promoter of the PDC regulatory gene. In a PDC-reduced yeast, one or more of (A)-(D) can be (i) mutated, (ii) disrupted, or (iii) deleted. Mutation, disruption or deletion of one or more of (A)-(D) can, in certain embodiments, contribute to a decrease (and/or lack) of pyruvate decarboxylase activity.

Many yeast strains contain more than one PDC gene. According to the present disclosure, a PDC-reduced yeast can be obtained by inhibition, reduction, or substantial elimination of any one, or any set, of PDC polypeptides in a cell. For example, wild-type S. cerevisiae strains contain Pdc1, Pdc5 and Pdc6 polypeptides all of which possess pyruvate decarboxylase activity. The transcription factor, Pdc2 is required for normal expression of Pdc1 and Pdc5. In certain embodiments, the PDC-reduced strain comprises modifications to reduce one or more of Pdc1, Pdc2, Pdc5, and Pdc6 activities. In other embodiments, the PDC-reduced strain comprises modifications to decrease each of Pdc1, Pdc5 and Pdc6 activities. In further embodiments, the PDC-reduced strain comprises modifications to decrease each of Pdc1 and Pdc5 activities.

In one embodiment, the PDC-reduced yeast is S. cerevisiae strain TAM (“MATa pdc1(-6,-2)::loxP pdc5(-6,-2)::loxP pdc6(-6,-2)::loxP ura3-52” ura-yeast having no detectable pyruvate decarboxylase activity, C₂ carbon source independent, glucose tolerant). In certain other embodiments, the PDC-reduced yeast is RWB837 (MATa ura3-52 pdc1::loxP pdc5::loxP pdc6::loxP) or strains descended from either of m850 or Lp4f. Both m850 and Lp4f were generated from a RWB837-derived strain (RWB876) through serial passaging and enriching for C₂ carbon source independent and glucose tolerant growth.

A “pyruvate carboxylase (PYC) polypeptide” can be any enzyme that uses a HCO3⁻ substrate to catalyze an ATP-dependent conversion of pyruvate to oxaloacetate (EC 6.4.1.1). PYC polypeptides contain a covalently attached biotin prosthetic group, which serves as a carrier of activated CO₂. In most instances, the activity of PYC polypeptides depends on the presence of acetyl-CoA. Biotin is not carboxylated (on PYC) unless acetyl-CoA (or a closely related acyl-CoA) is bound to the enzyme. Aspartate often serves as an inhibitor of PYC polypeptides. PYC polypeptides are generally active in a tetrameric form.

A polypeptide need not be identified in the literature as a pyruvate carboxylase at the time of filing of the present application to be within the definition of a PYC polypeptide. A PYC from any source organism may be used in accordance with the present disclosure, and the PYC may be wild type or modified from wild type. For example, the PYC can be a S. cerevisiae pyruvate carboxylase.

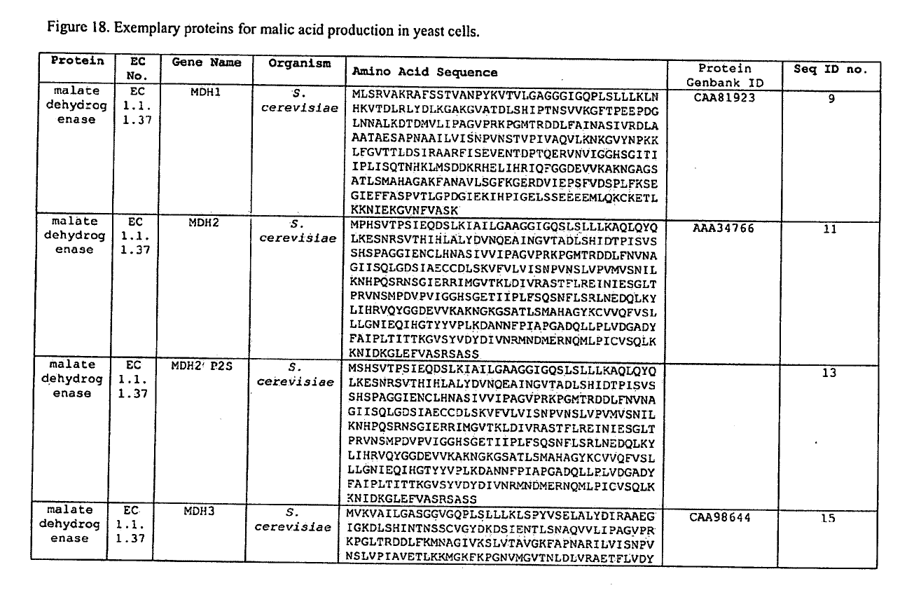

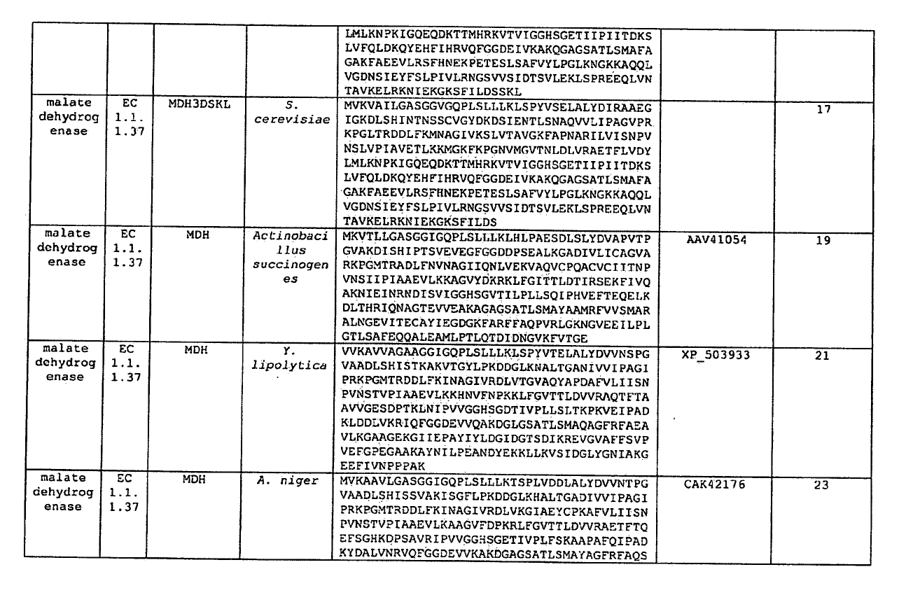

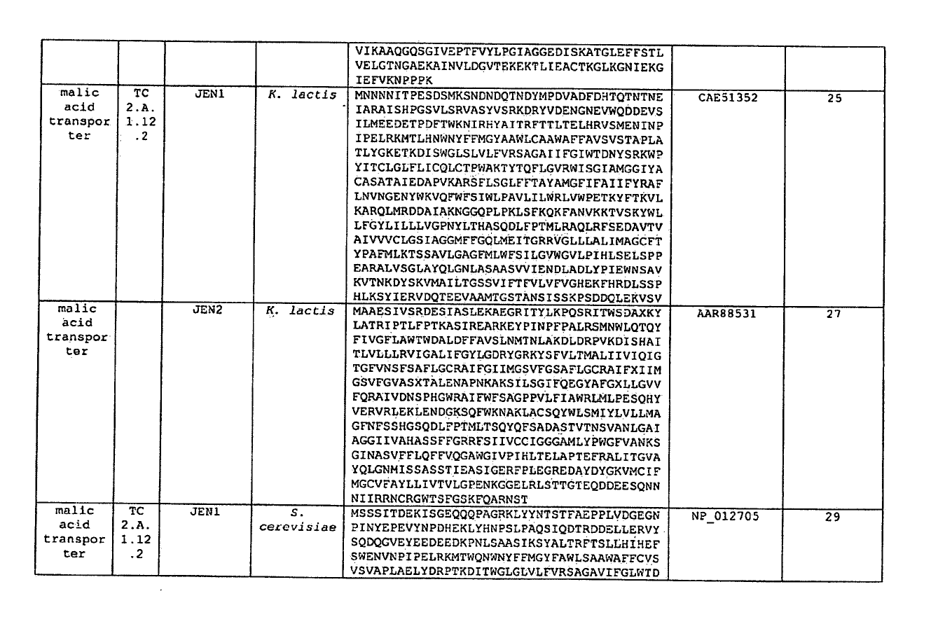

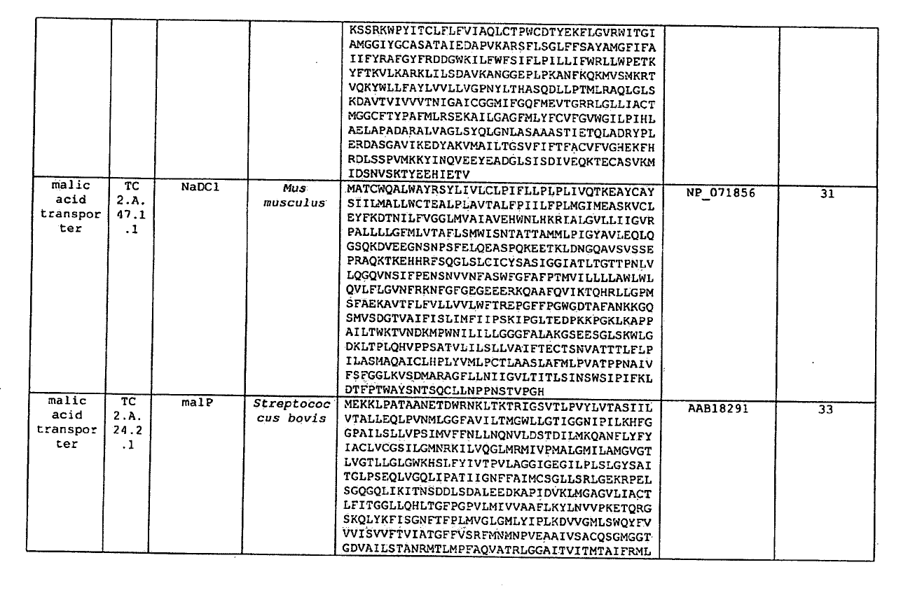

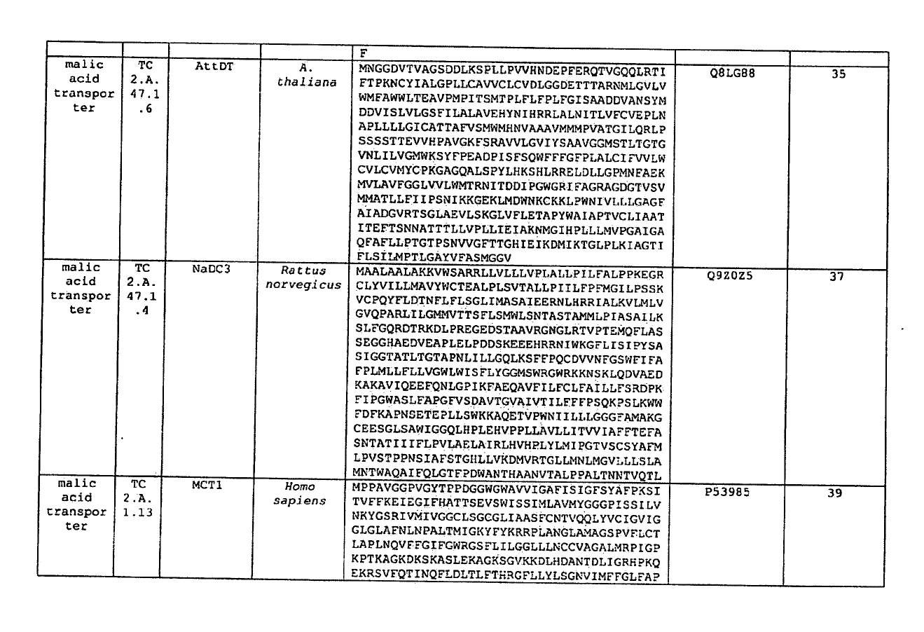

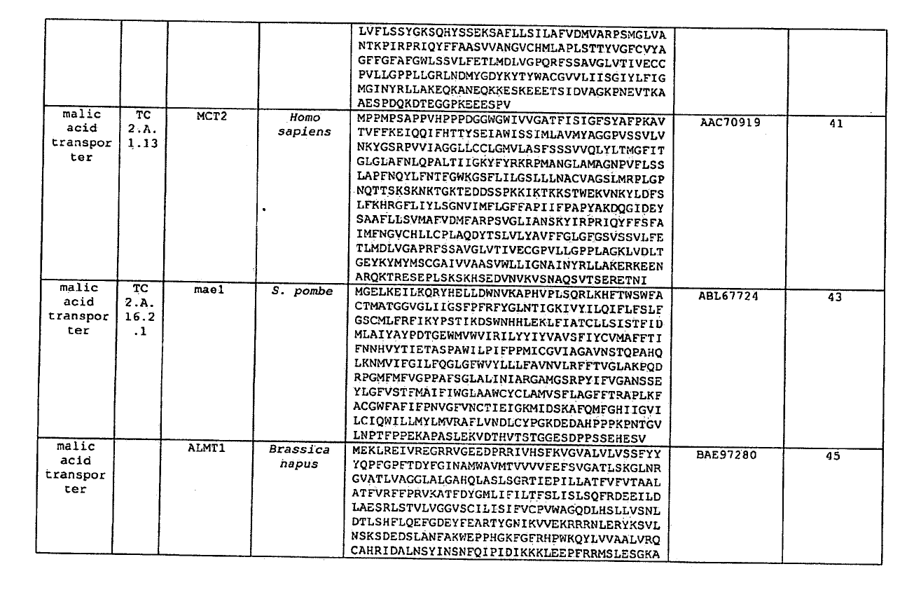

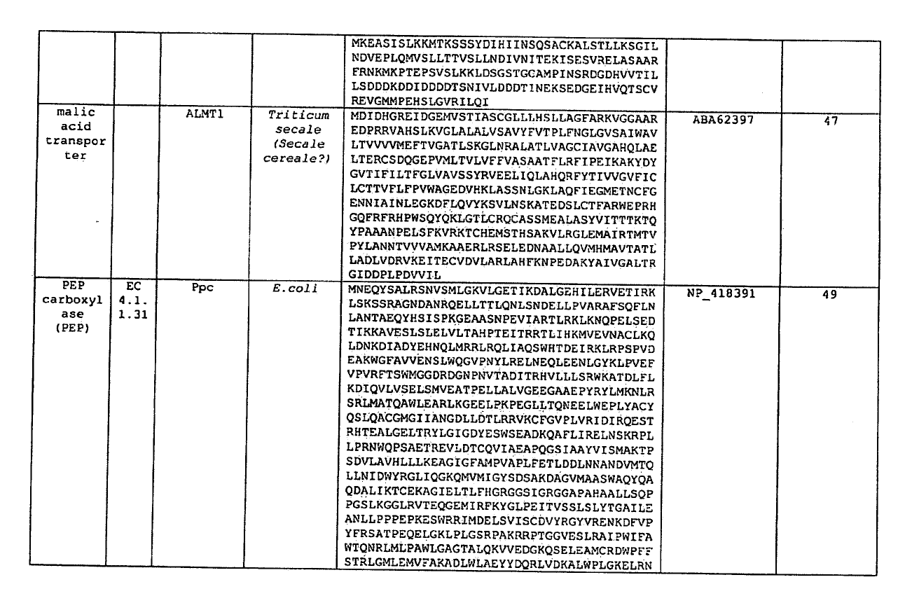

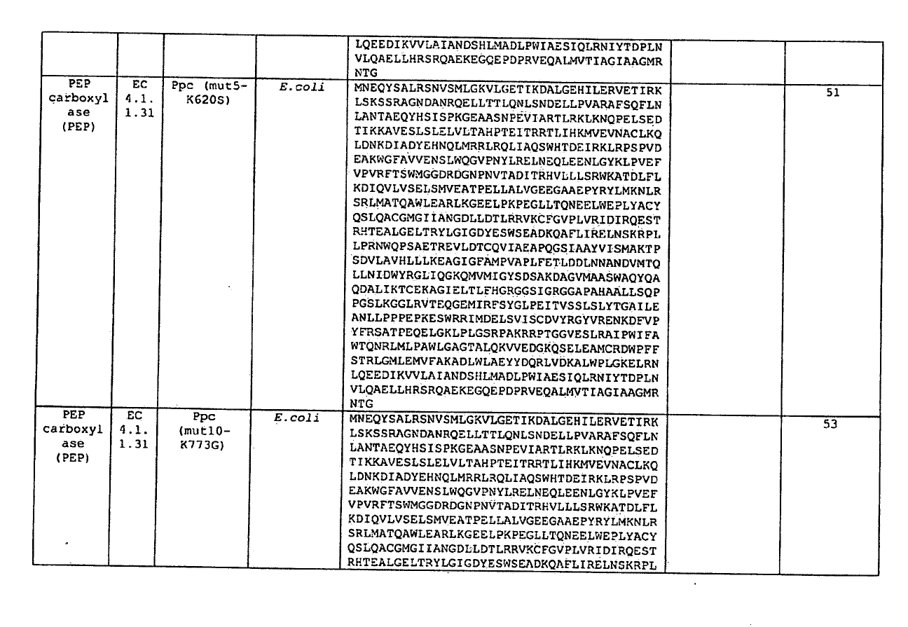

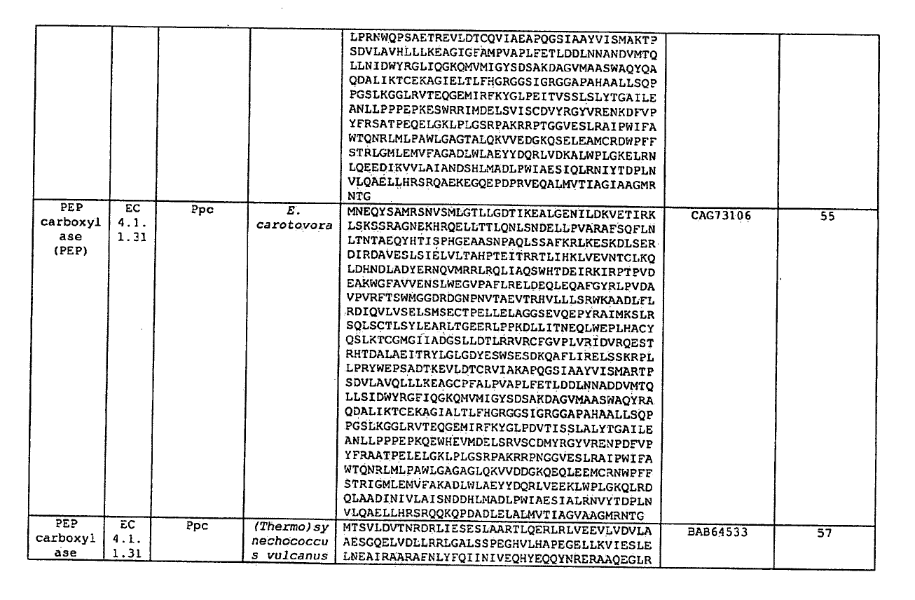

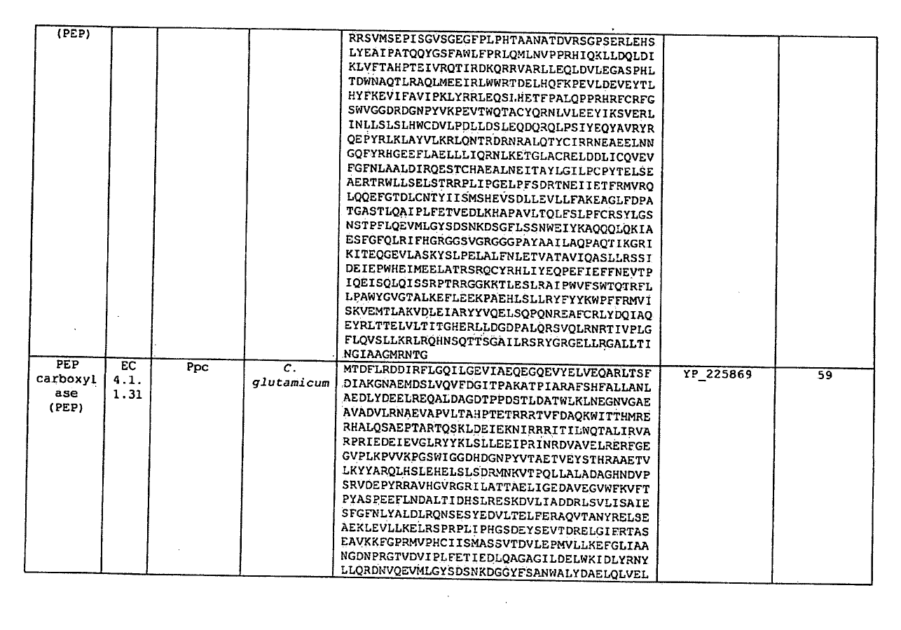

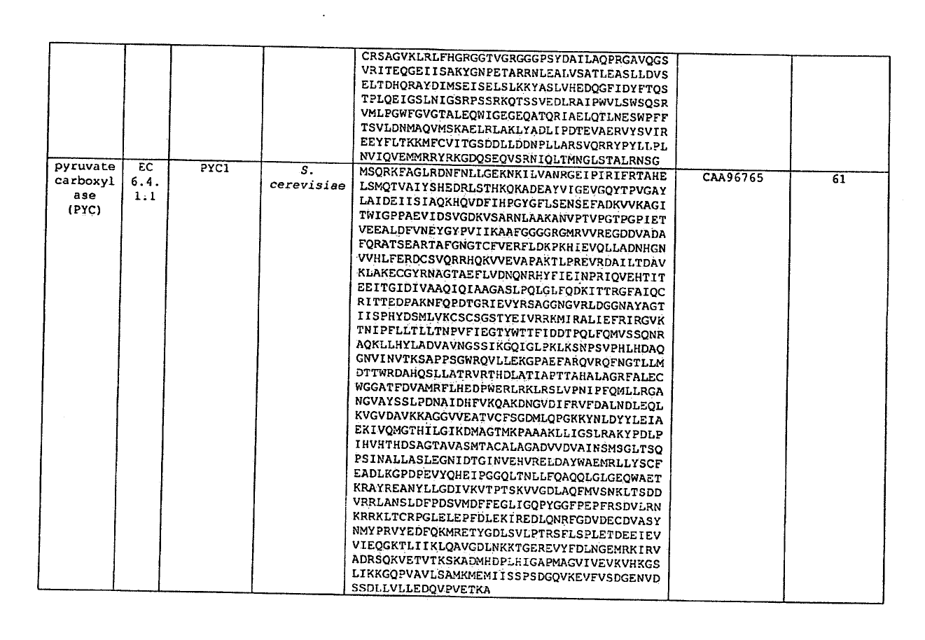

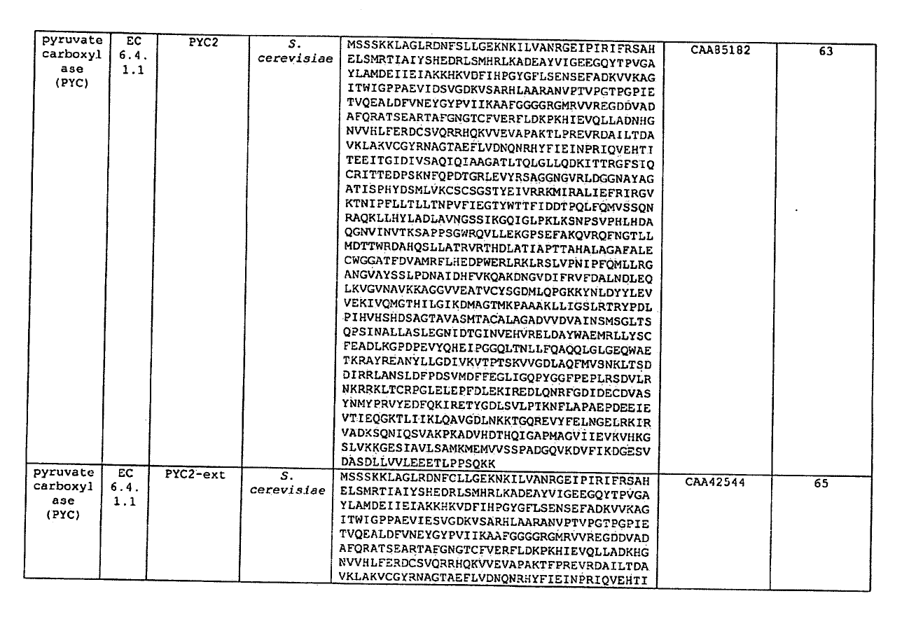

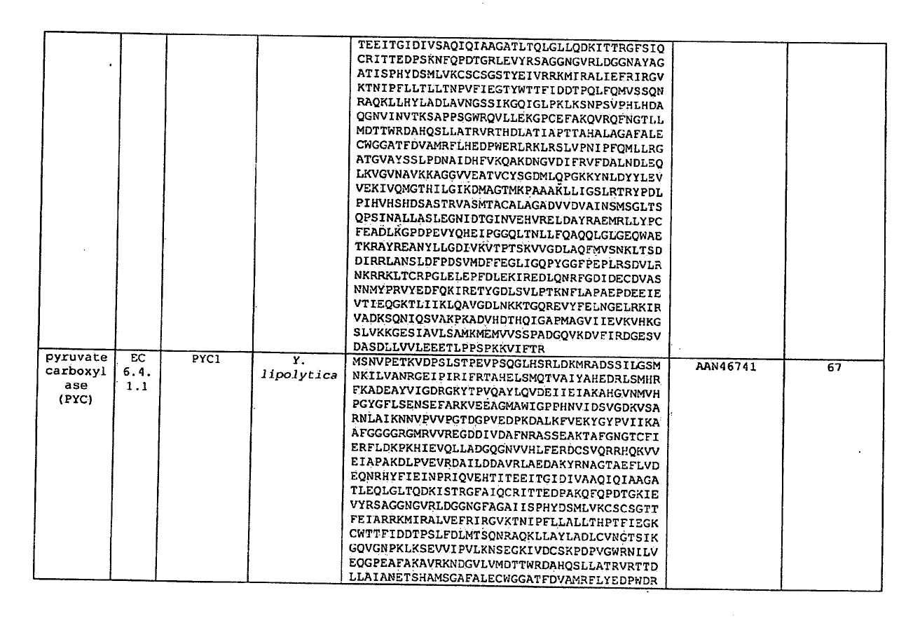

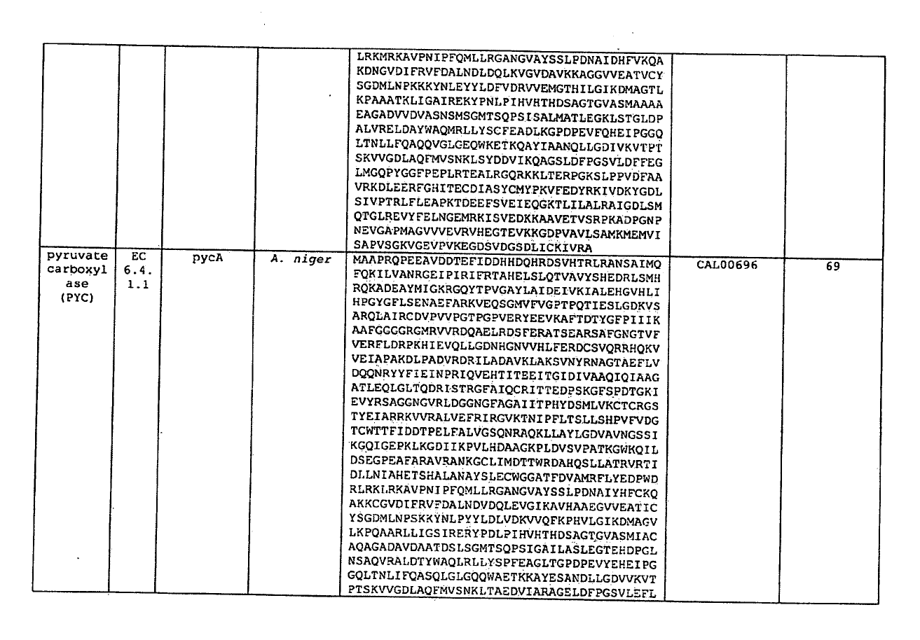

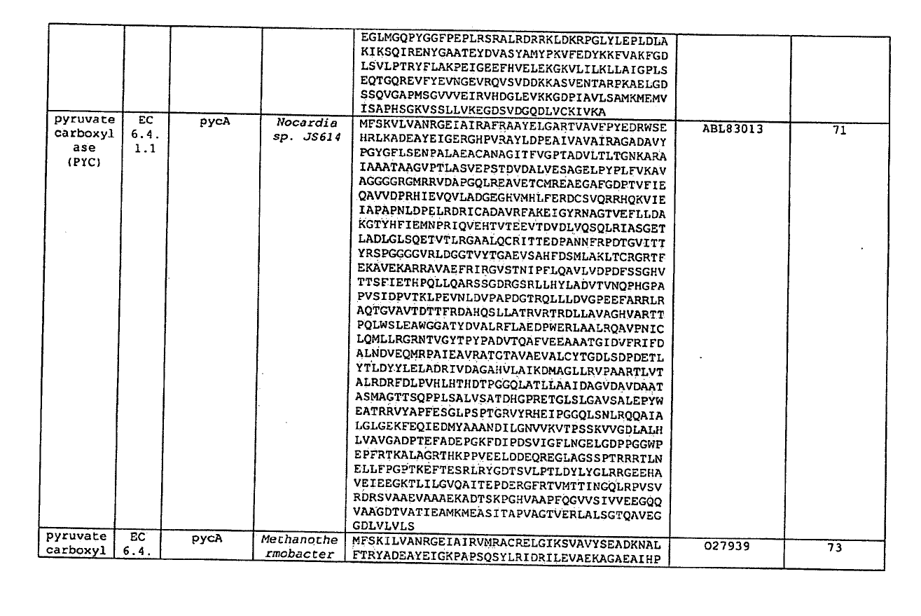

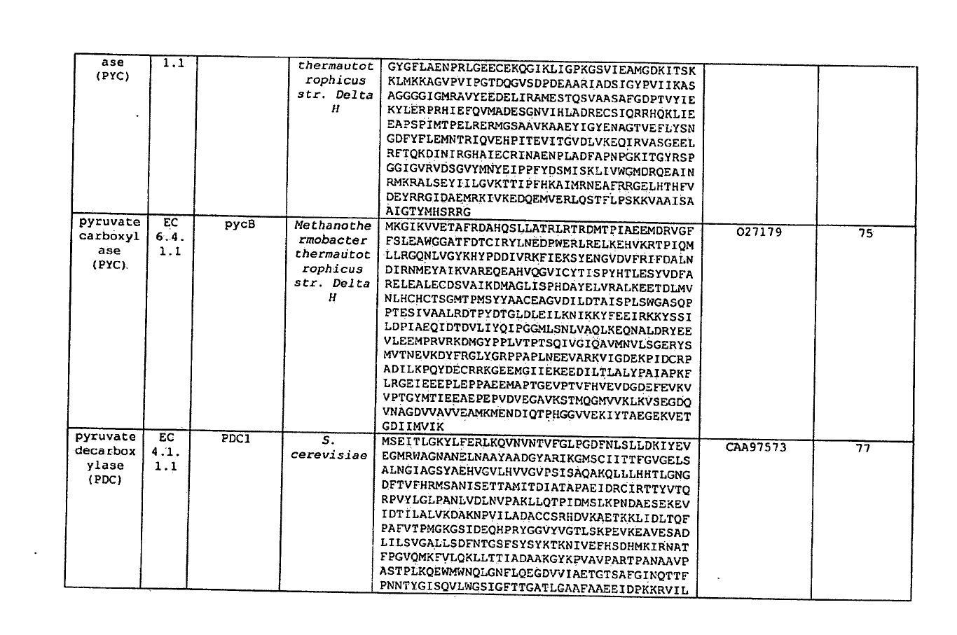

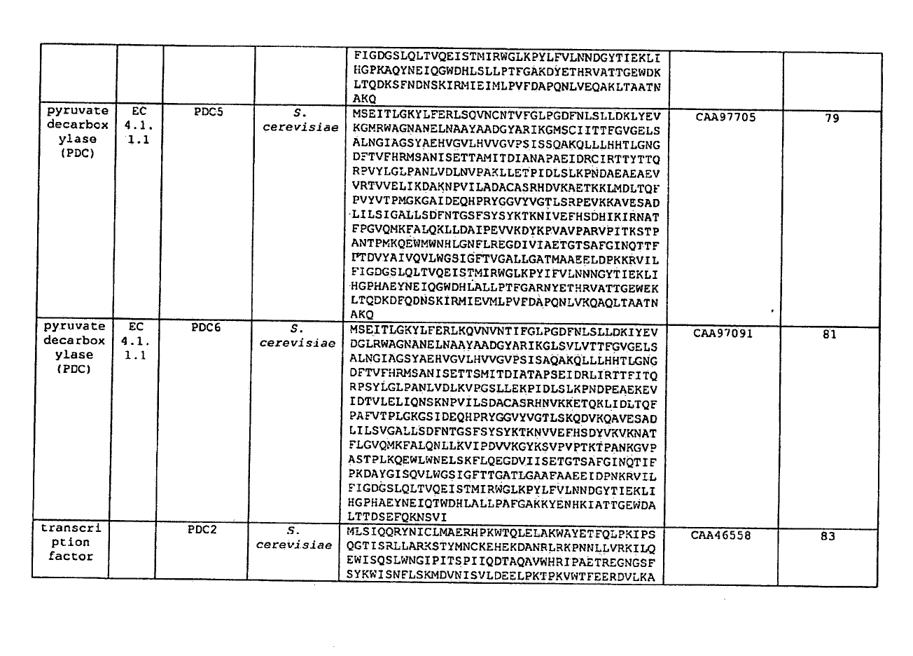

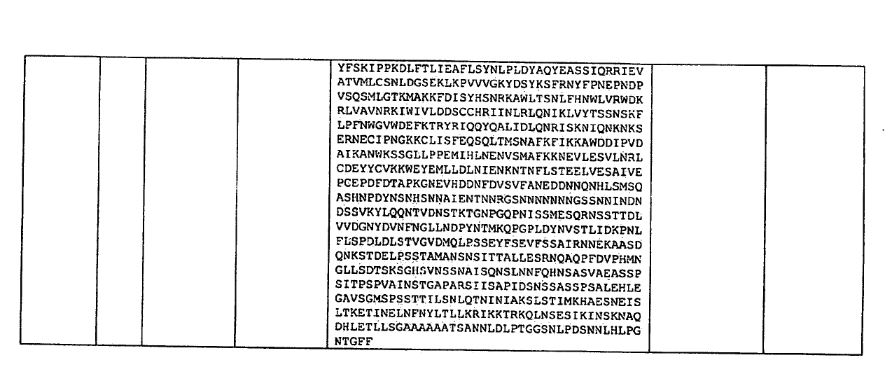

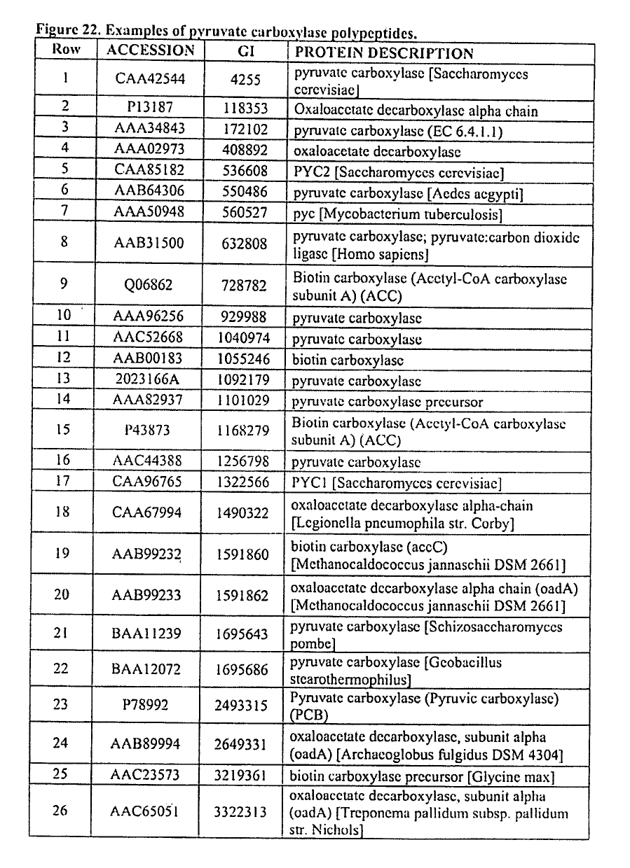

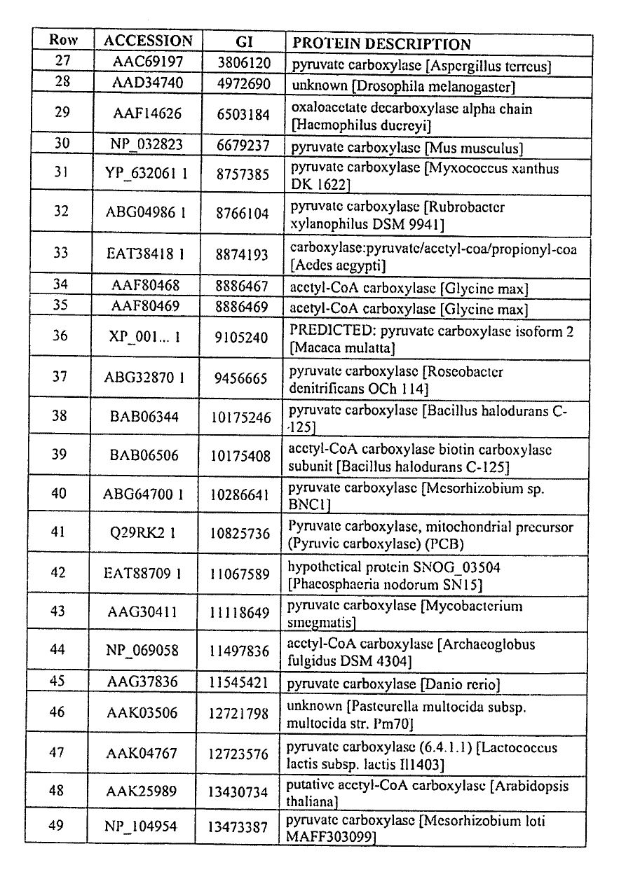

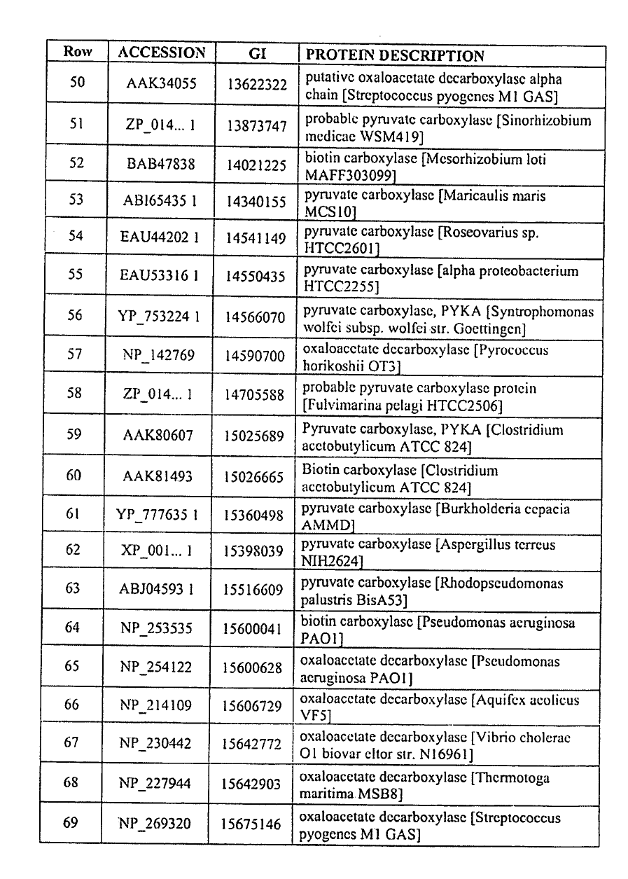

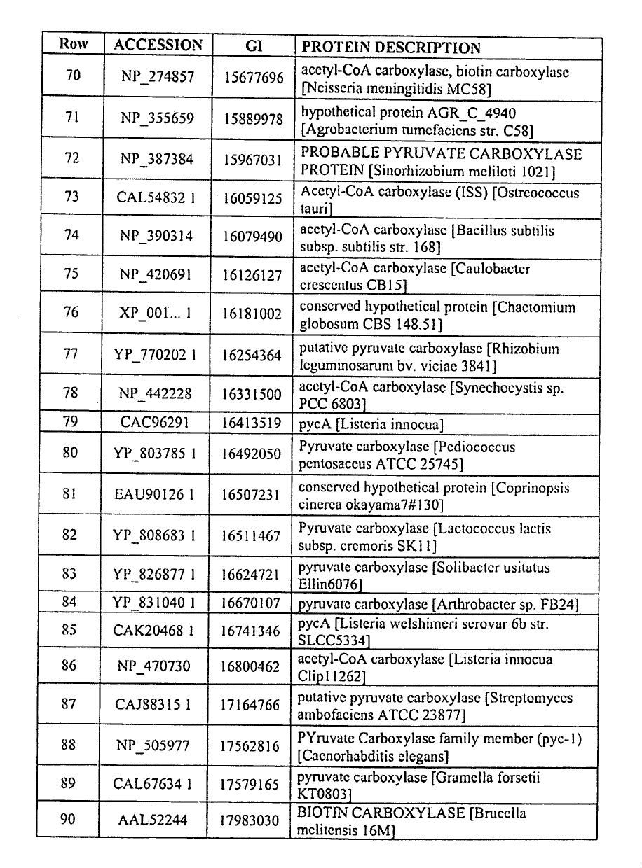

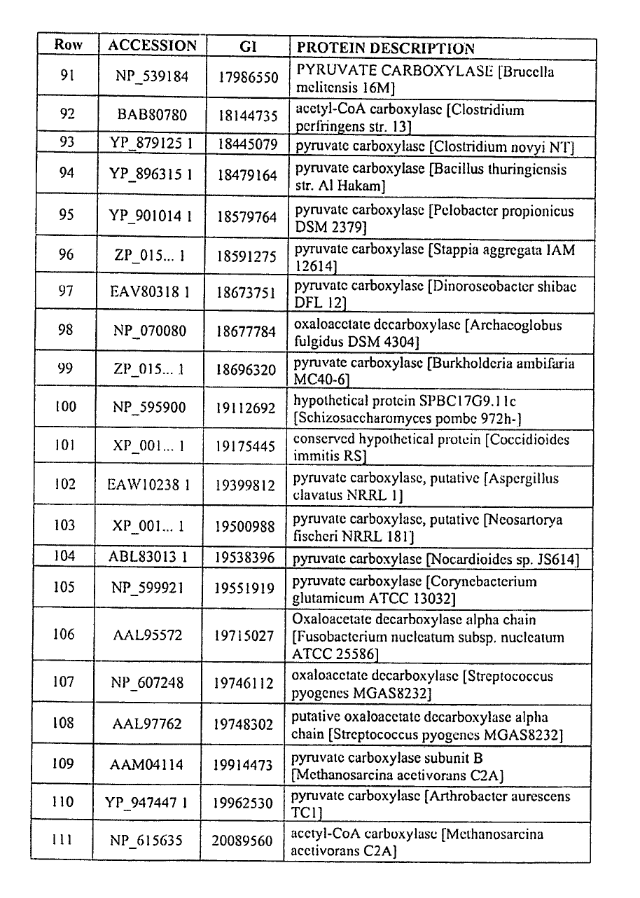

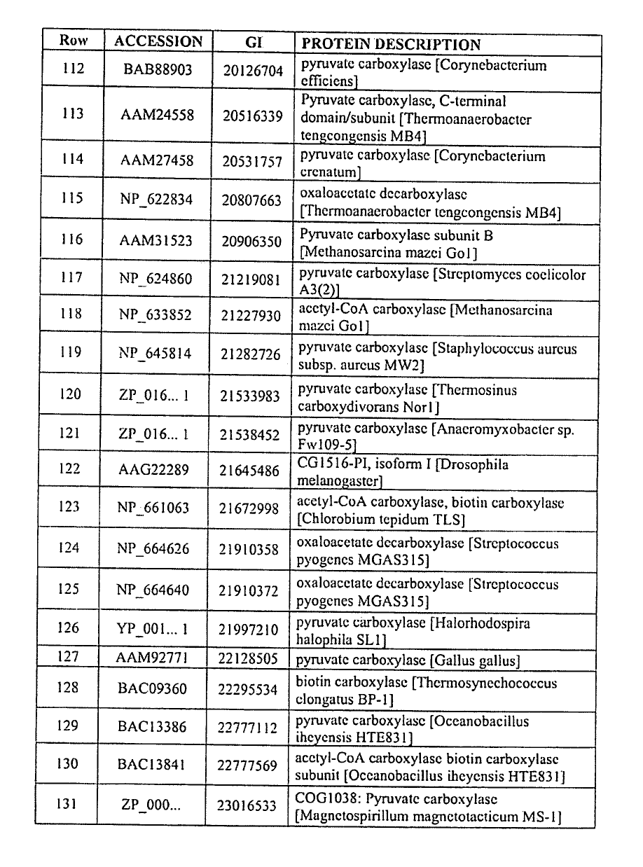

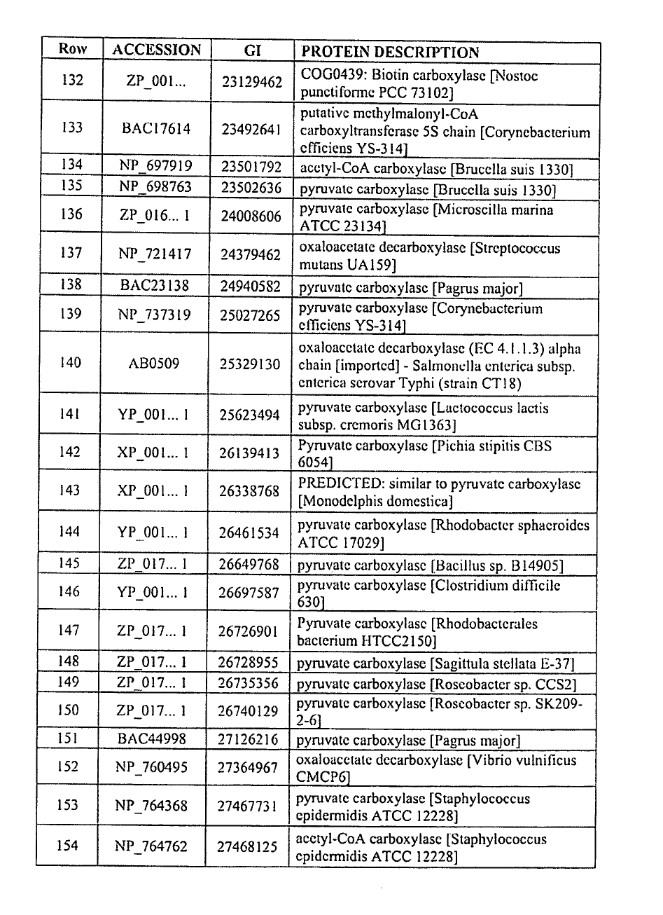

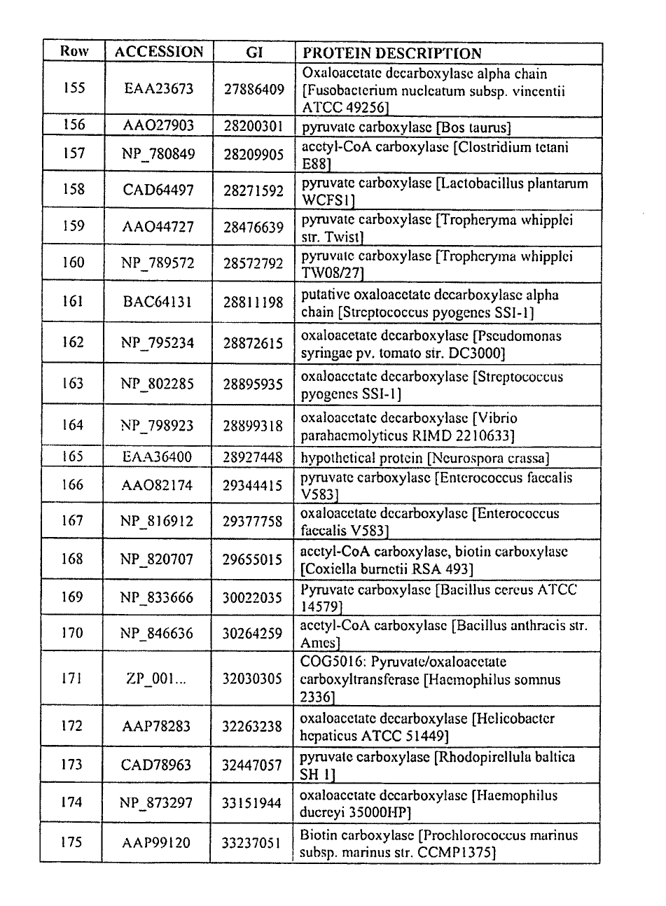

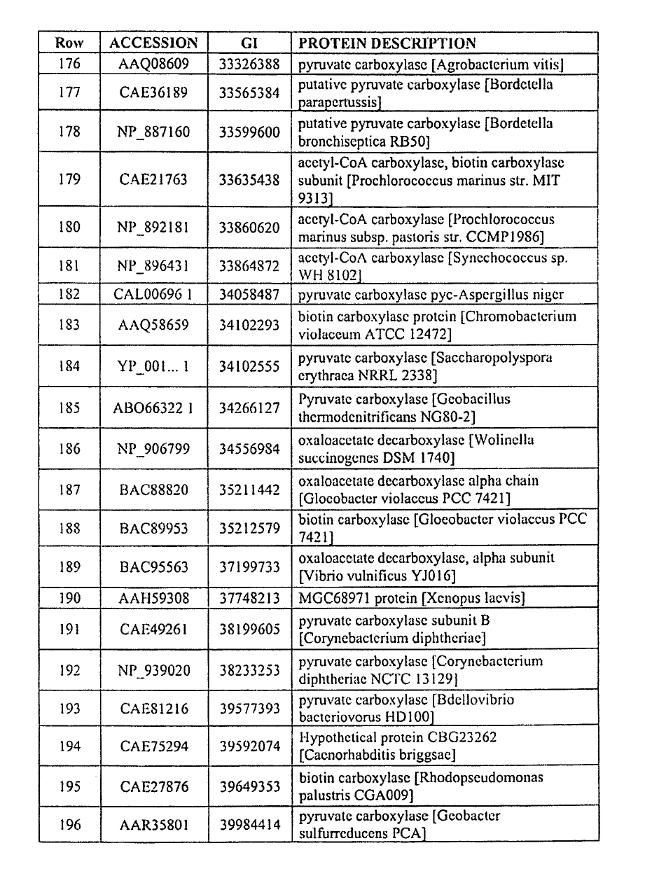

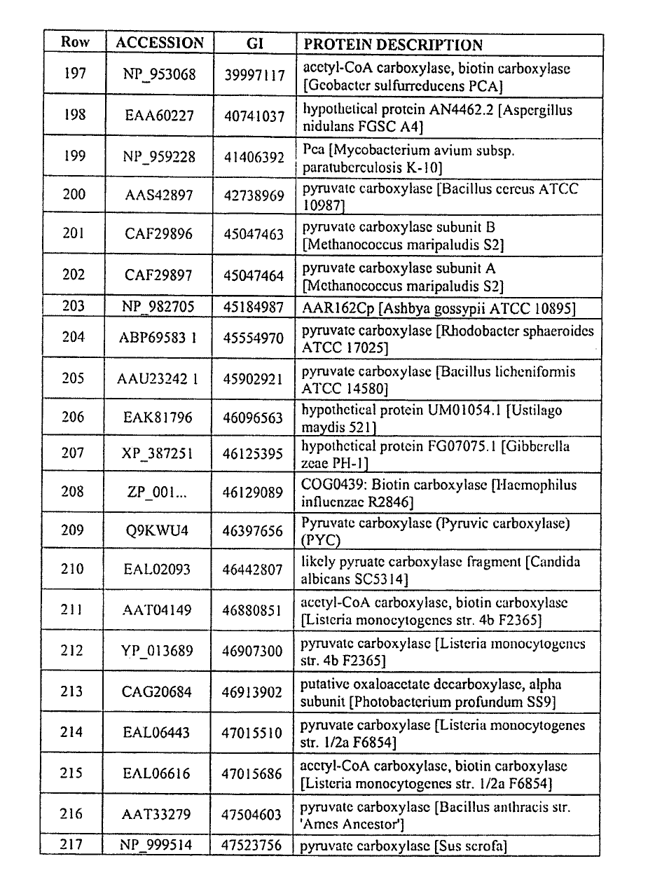

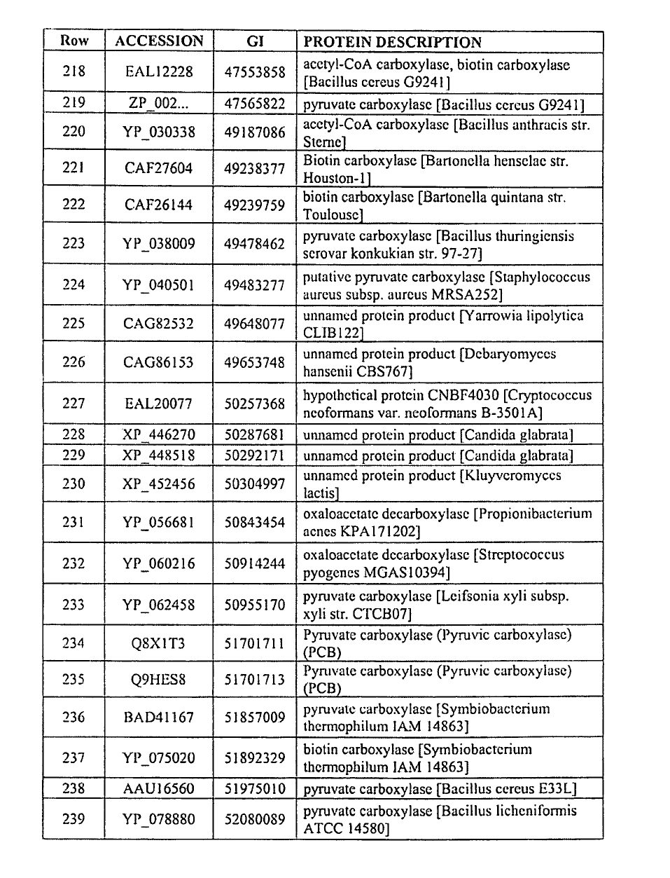

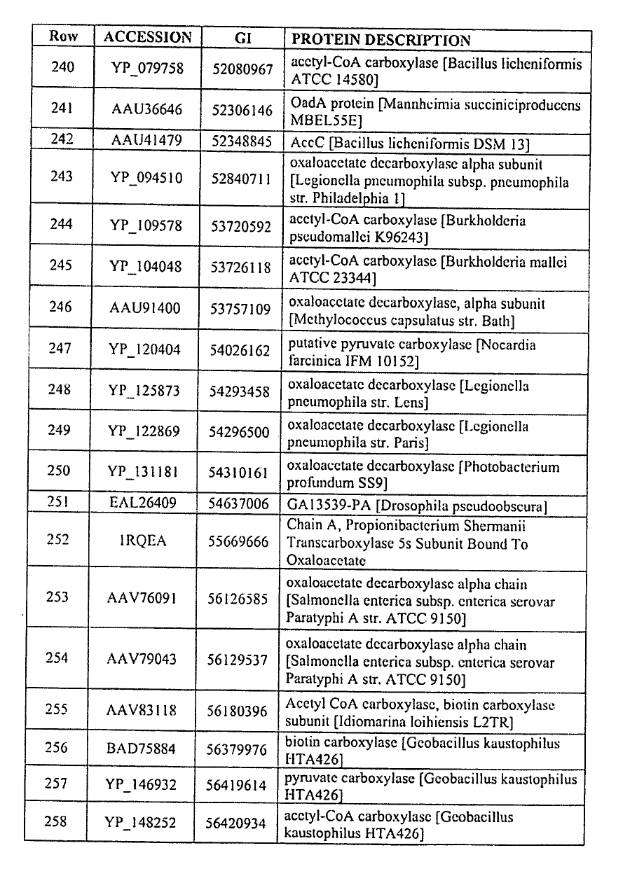

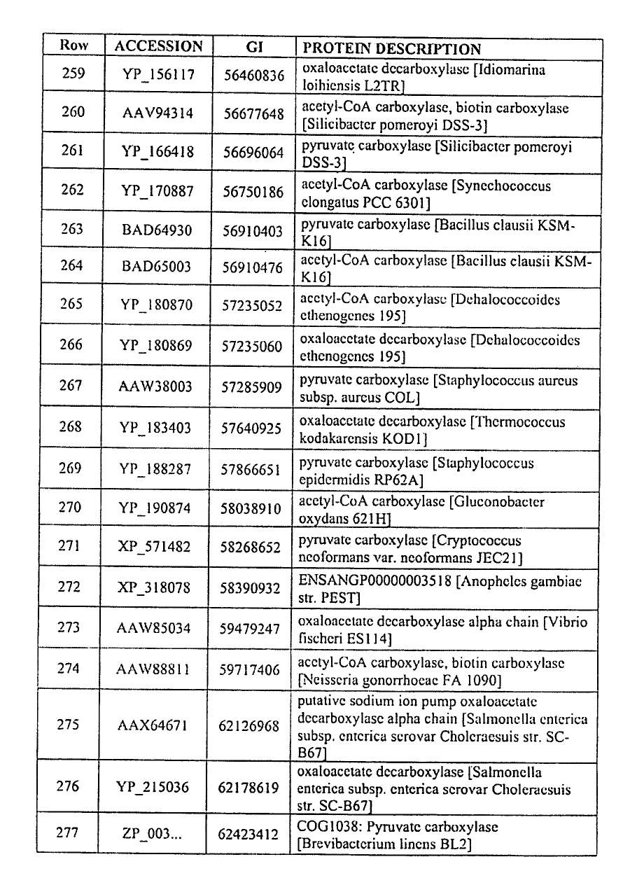

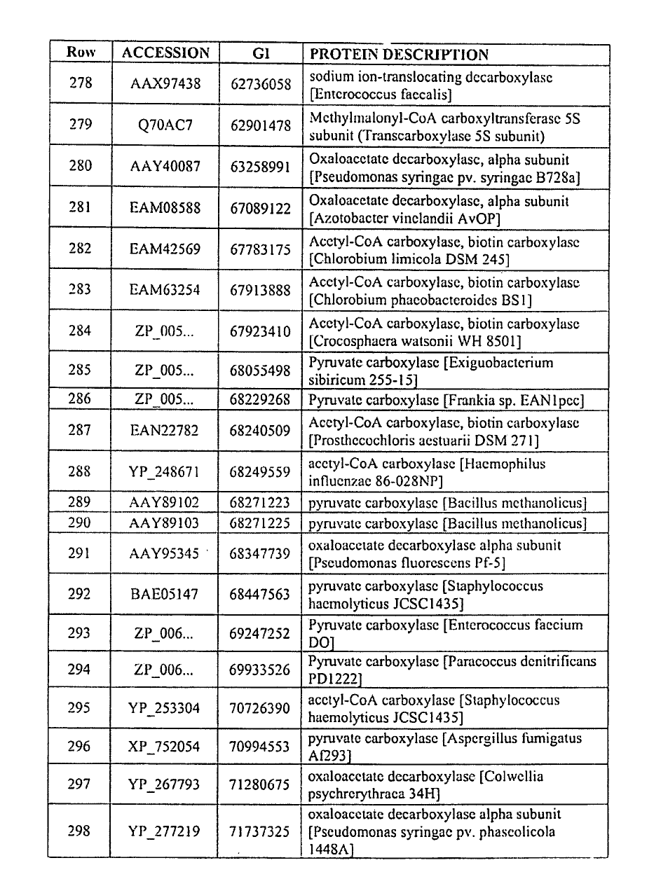

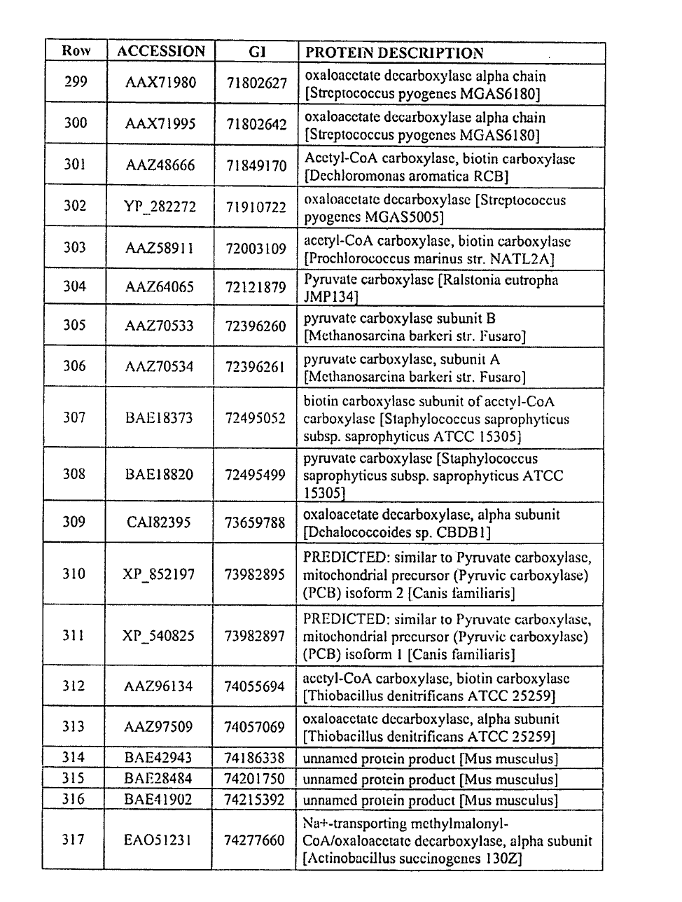

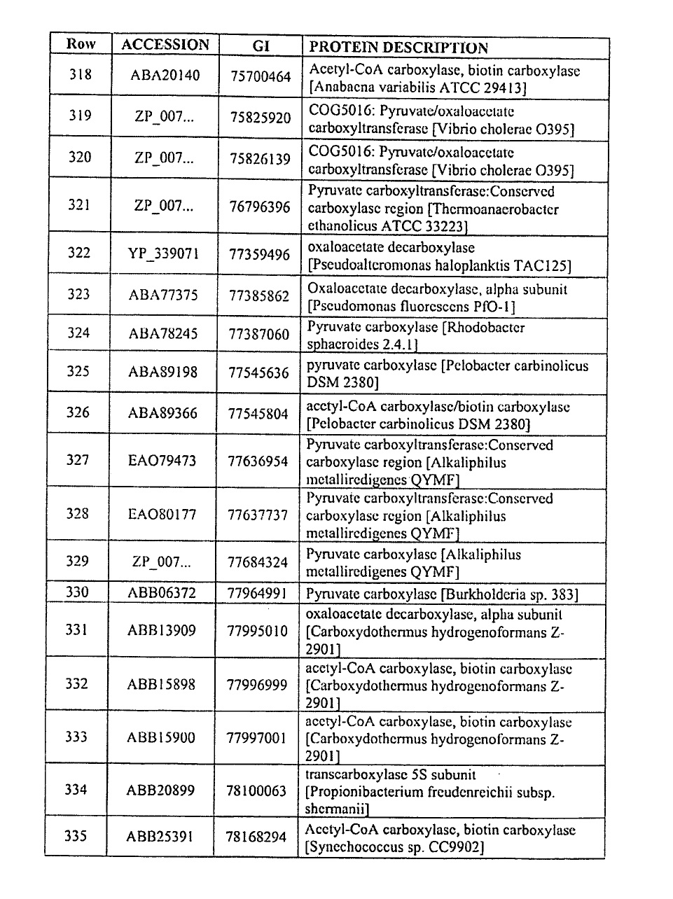

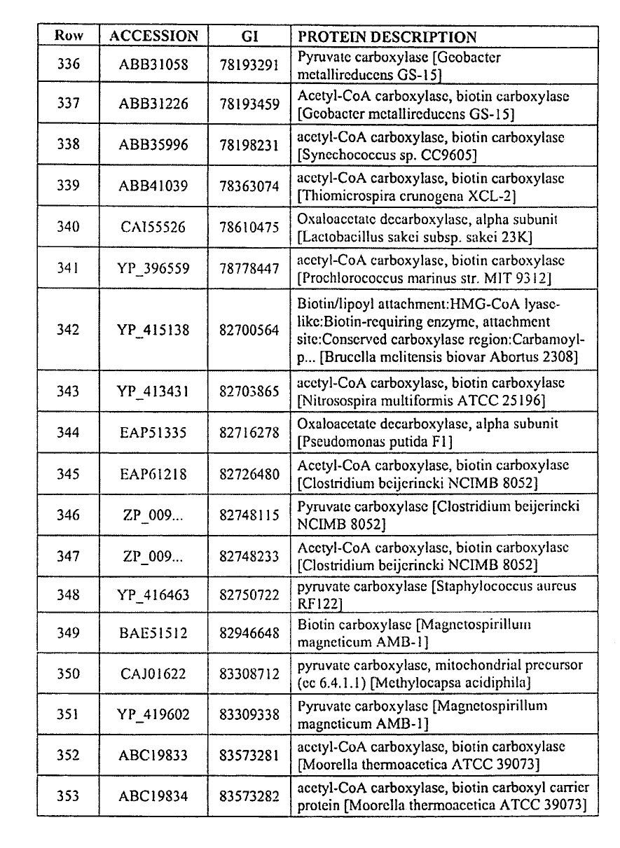

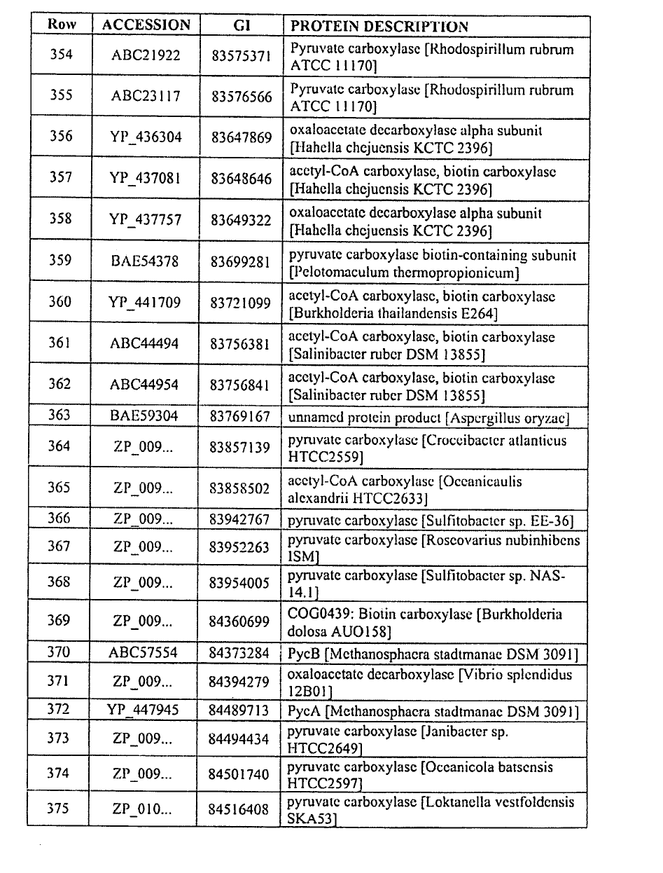

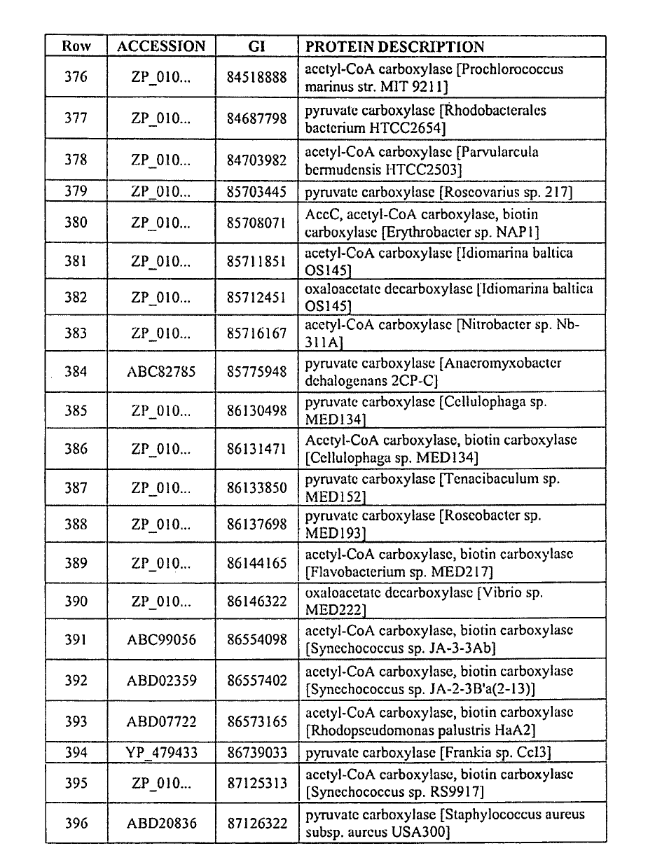

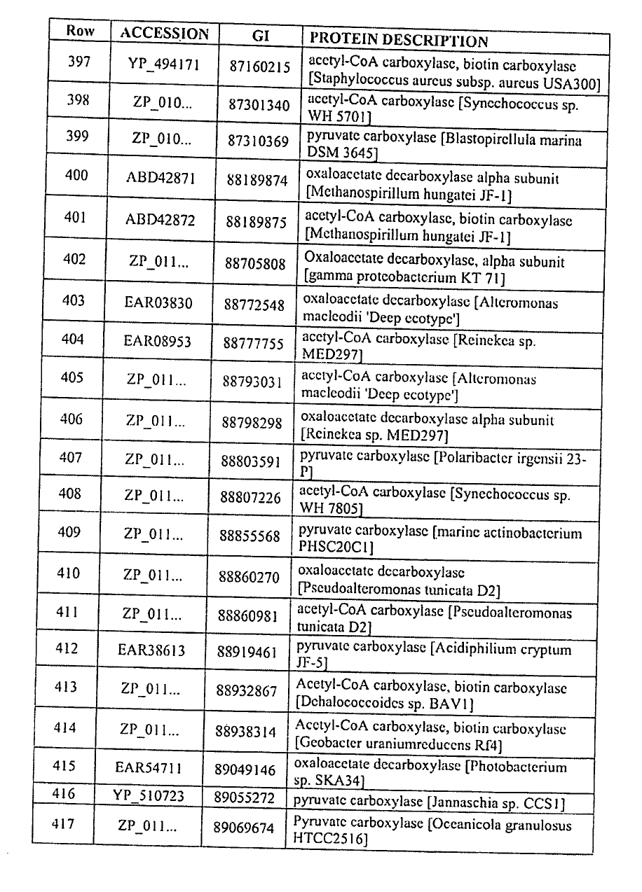

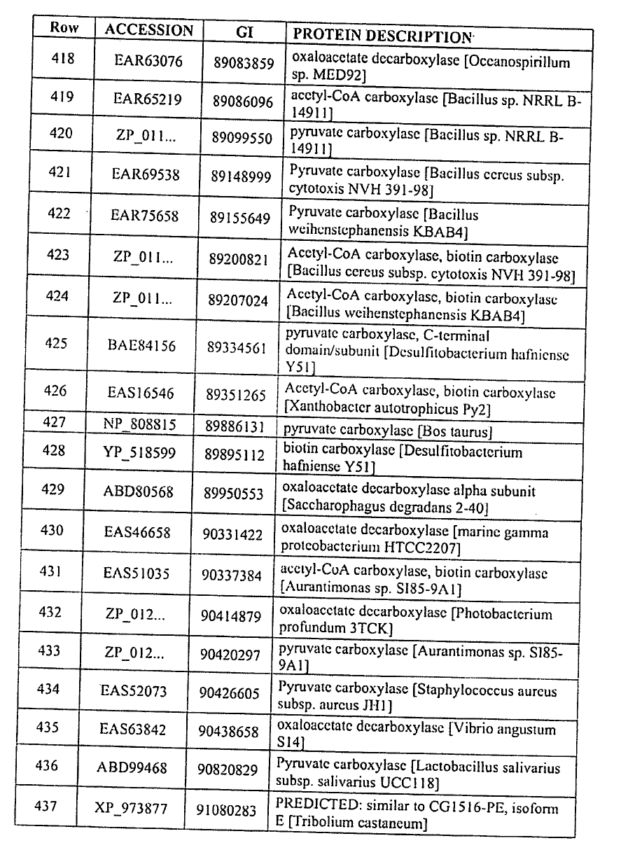

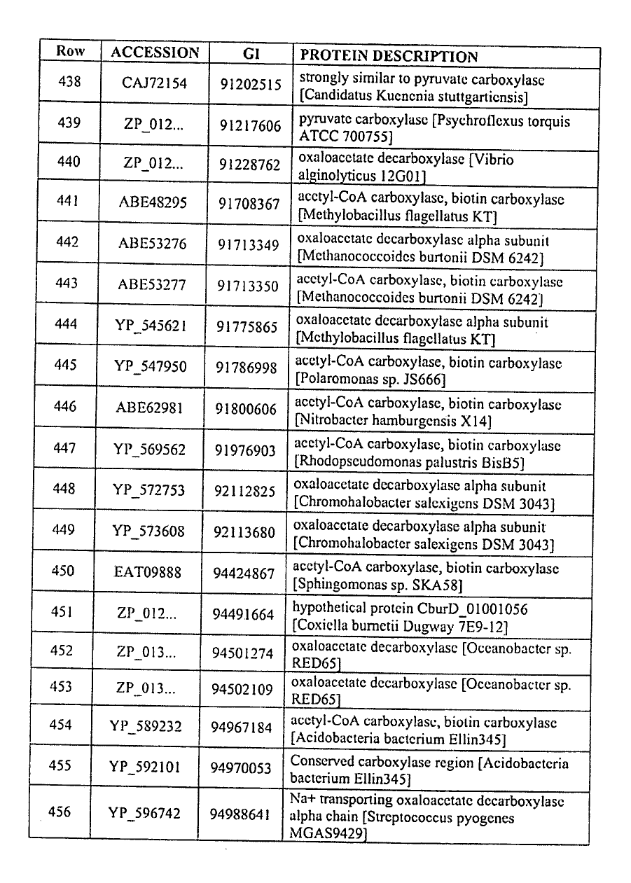

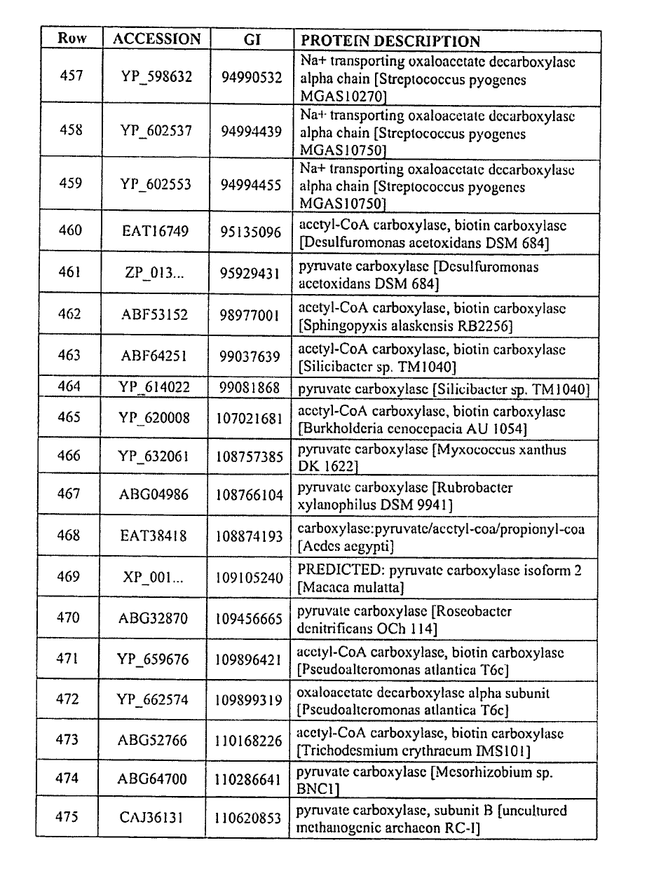

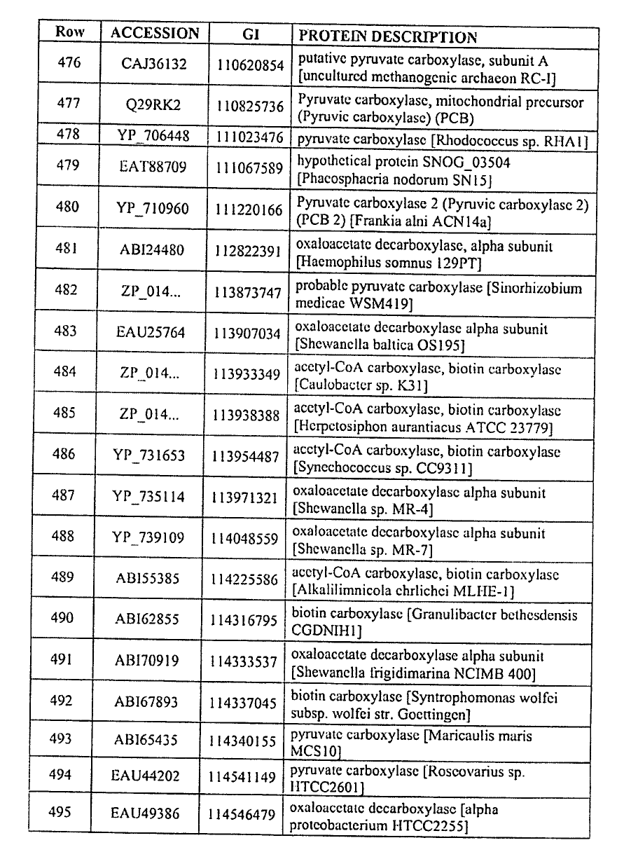

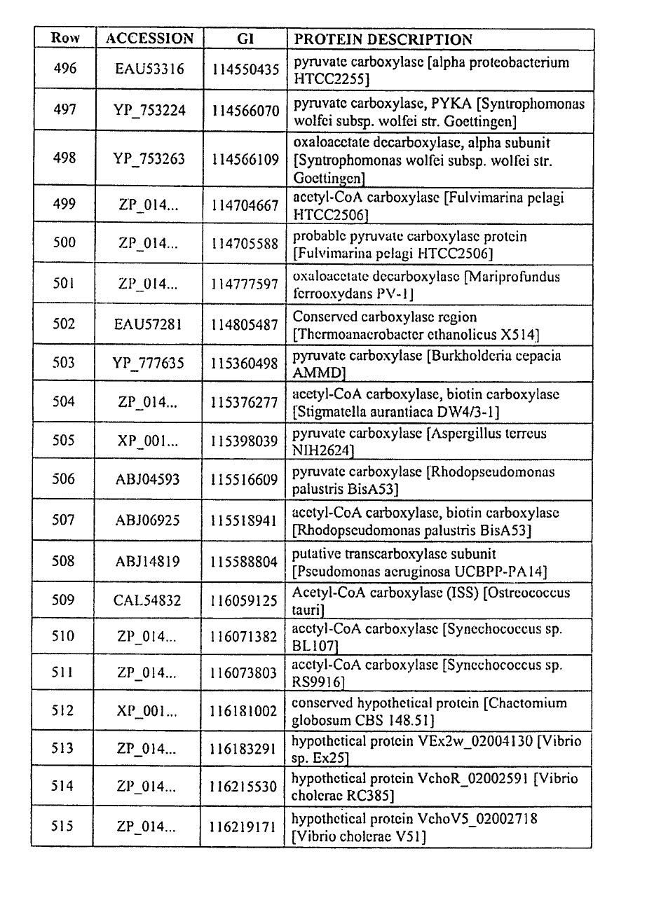

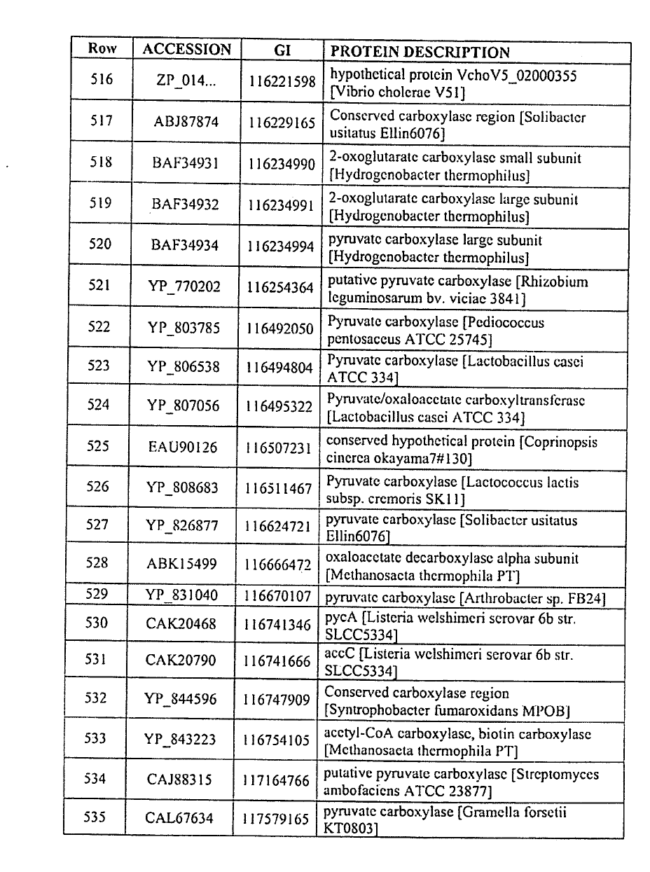

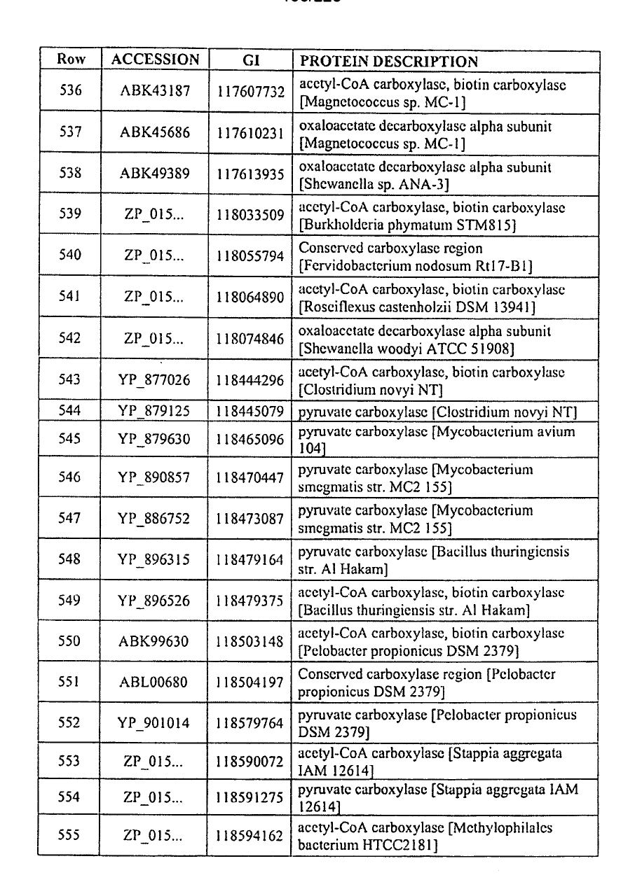

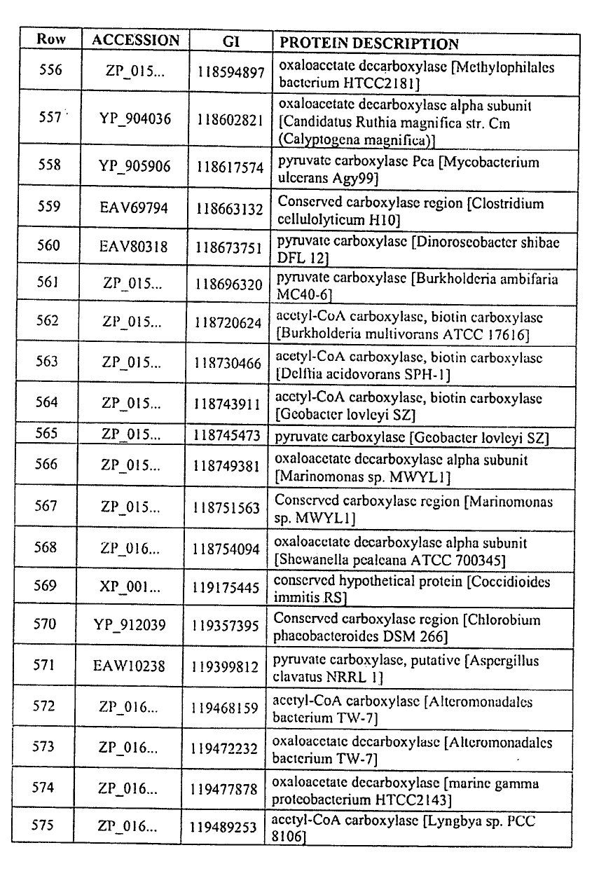

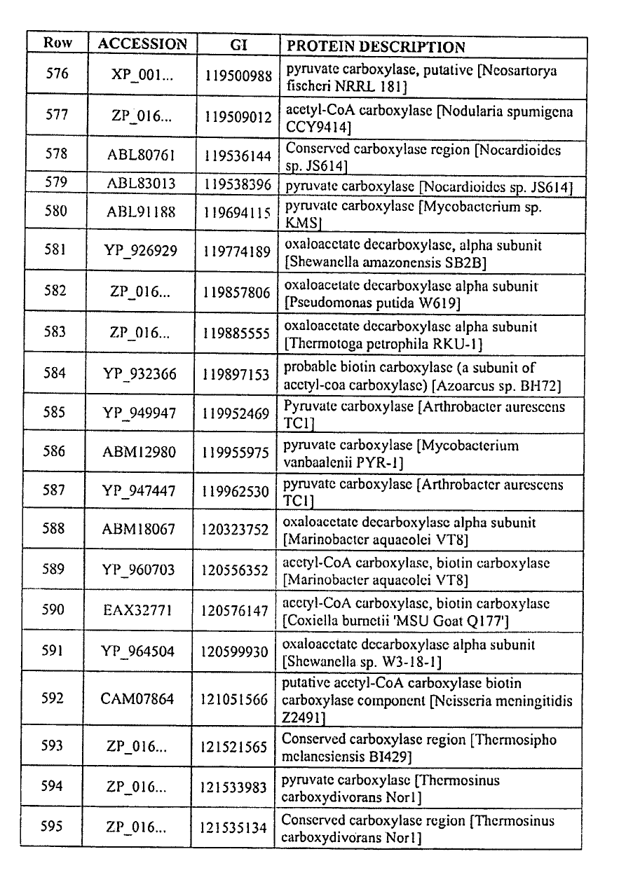

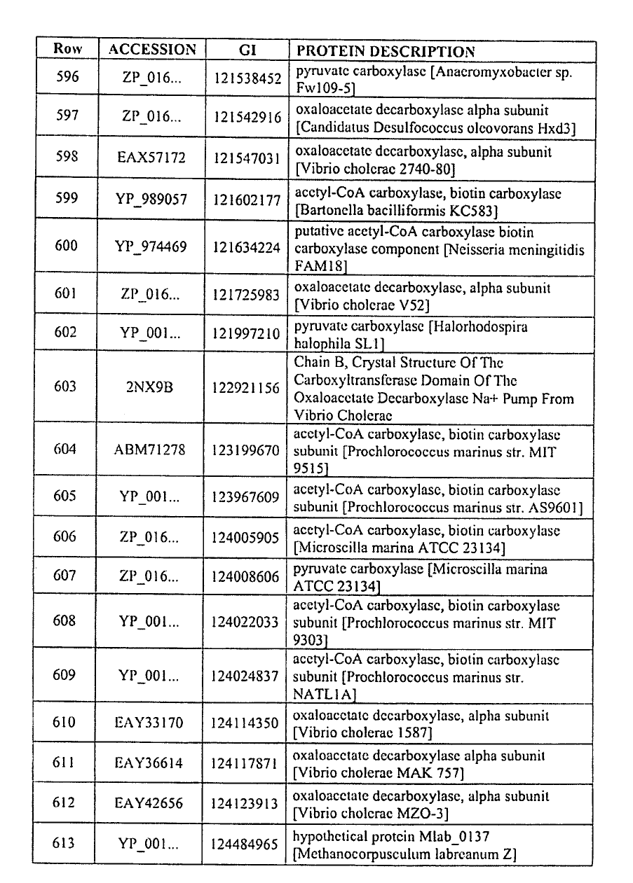

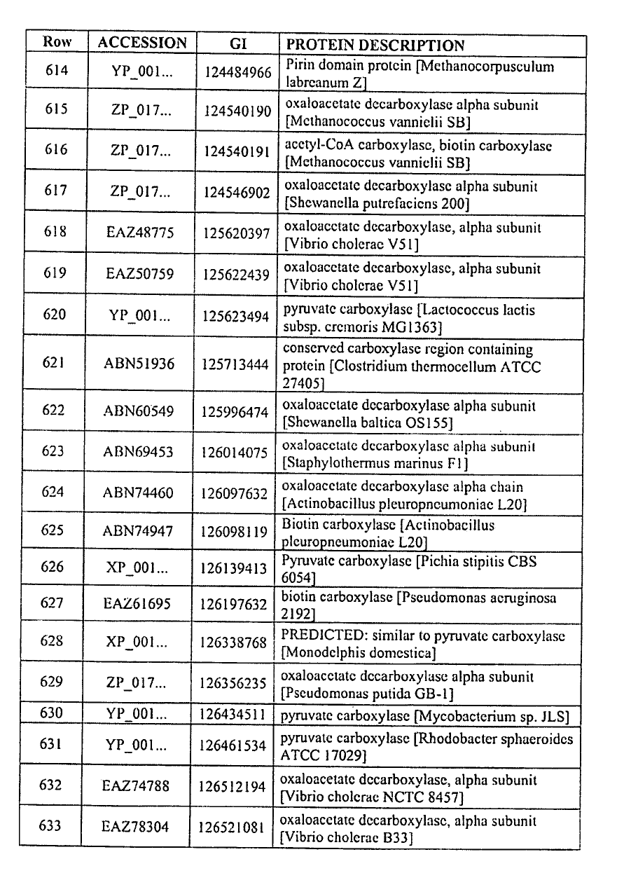

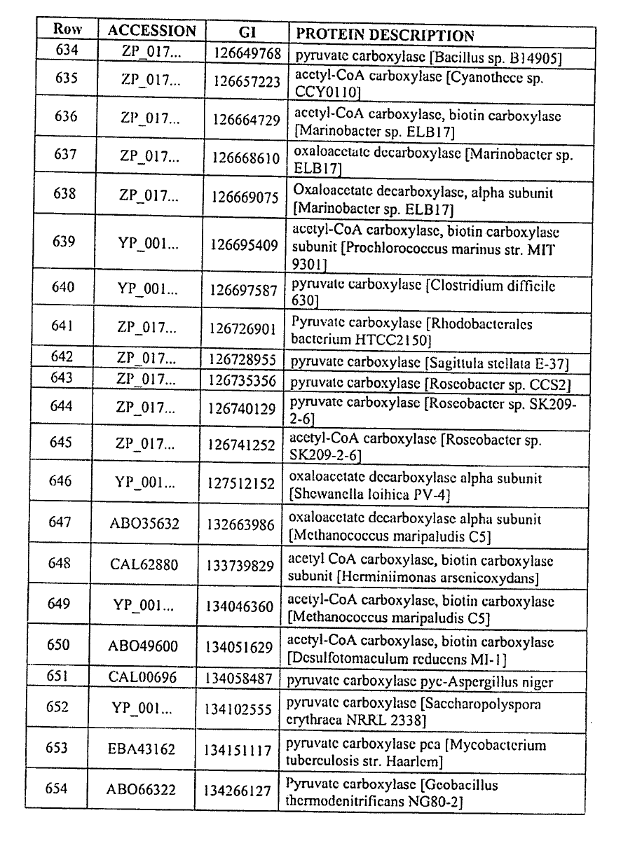

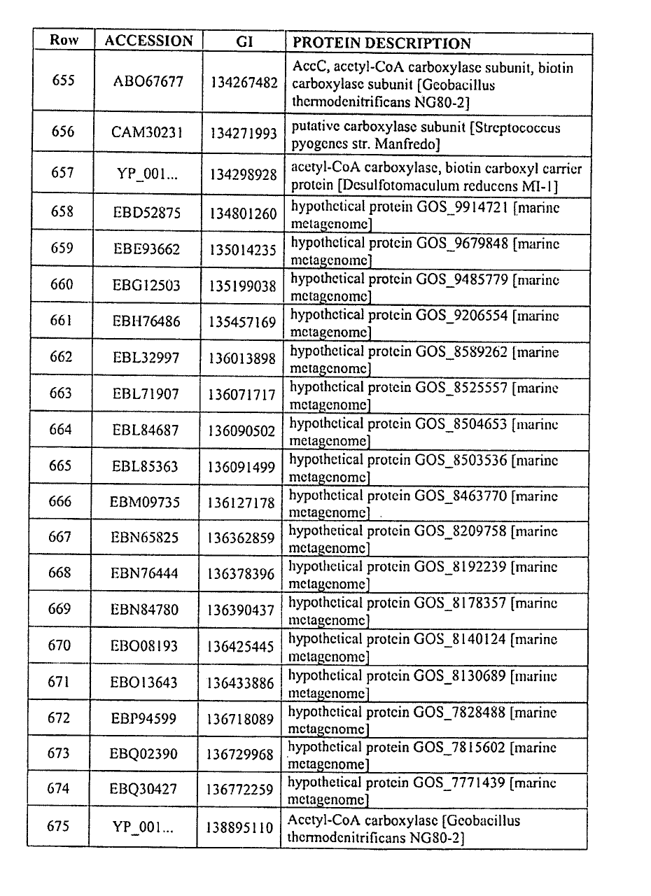

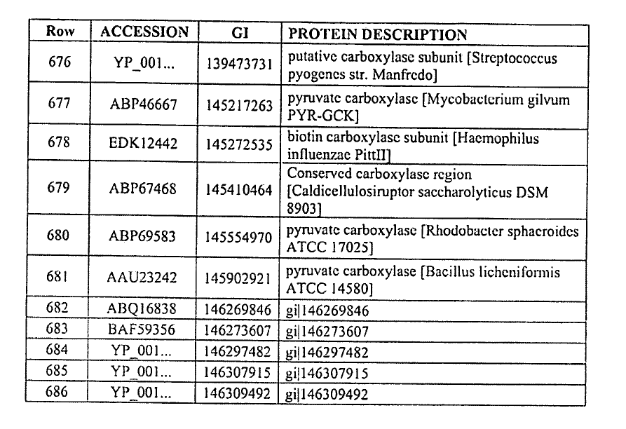

In one embodiment, a PYC polypeptide is a PYC that has at least 75% identity to the amino acid sequence given in SEQ ID NO:1. In one embodiment, the PYC has at least 80% identity to the amino acid sequence given in SEQ ID NO:1. In one embodiment, the PYC has at least 85% identity to the amino acid sequence given in SEQ ID NO:1. In one embodiment, the PYC has at least 90% identity to the amino acid sequence given in SEQ ID NO:1. In one embodiment, the PYC has at least 95% identity to the amino acid sequence given in SEQ ID NO:1. In another embodiment, the PYC has at least 96% identity to the amino acid sequence given in SEQ ID NO:1. In an additional embodiment, the PYC has at least 97% identity to the amino acid sequence given in SEQ ID NO:1. In yet another embodiment, the PYC has at least 98% identity to the amino acid sequence given in SEQ ID NO:1. In still another embodiment, the PYC has at least 99% identity to the amino acid sequence given in SEQ ID NO:1. In still yet another embodiment, the PYC has the amino acid sequence given in SEQ ID NO:1. In another embodiment, the PYC polypeptide has the amino acid sequence of a pyruvate carboxylase in FIG. 18 or has at least 99%, 98%, 97%, 96%, 95%, 94%, 93%, 92%, 91%, 90%, 85%, 80%, 75% identity to a pyruvate carboxylase in FIG. 18. In another embodiment, the PYC polypeptide is a polypeptide represented by the Genbank GI numbers in FIG. 22 or has at least 99%, 98%,97%, 96%, 95%, 94%, 93%, 92%,.91%, 90%, 85%, 80%, 75% identity toe polypeptide represented by the Genbank GI numbers in FIG. 22. In various embodiments the the PYC has at least 99%, 98%, 97%, 96%, 95%, 94%, 93%, 92%, 91%, 90%, 85%, 80%, 75% to Yarrowia lipolytic PYC.

Identity can be determined by a sequence alignment. As is known in the art, sequence alignment typically involves comparison of two sequences and determination of positions in which the sequences have the identical or similar amino acids. In some embodiments, gaps can be introduced in one or both of the sequences for optimal alignment, and non-identical sequences can be disregarded for comparison purpose. In certain embodiments, the length of a sequence aligned for comparison purposes is at least 30%, at least 40%, at least 50%, at least 60%, at least 70%, at least 80%, at least 90%, at least 95% or 100% of the length of the reference sequence. Residues at corresponding positions are then compared. When a position in the first sequence is occupied by the same residue as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences.

The comparison of sequences and determination of percent identity between two sequences can be accomplished using a mathematical algorithm. A variety of sequence alignment algorithms is known in the art.

For example, in some embodiments, the Needleman and Wunsch (1970) J. Mol. Biol. 48:444-453 algorithm can be utilized. This algorithm has been incorporated into the GAP program in the GCG software package (available at http://www.gcg.com). In some such embodiments, the Neddleman and Wunsch algorithim is employed using either a Blossum 62 matrix or a PAM250 matrix, and a gap weight of 16, 14, 12, 10, 8, 6, or 4 and a length weight of 1, 2, 3, 4, 5, or 6.

In some embodiments, sequence alignment is performed using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the disclosure) are a Blossum 62 scoring matrix with a gap penalty of 12, a gap extend penalty of 4, and a frameshift gap penalty of 5.

In some embodiments, a sequence alignment is performed using the algorithm of Meyers and Miller ((1989) CABIOS, 4:11-17). This algorithm has been incorporated into the ALIGN program (version 2.0). In some such embodiments, this agorithm is employed using a PAM120 weight residue table, a gap length penalty of 12 and a gap penalty of 4.

In some embodiments, a sequence alignment is performed using the ClustalW program. In some such embodiments, defalt values, namely: DNA Gap Open Penalty=15.0, DNA Gap Extension Penalty=6.66, DNA Matrix=Identity, Protein Gap Open Penalty=10.0, Protein Gap Extension Penalty=0.2, Protein matrix=Gonnet, and employed. Identity can be calculated according to the procedure described by the ClustalW documentation: “A pairwise score is calculated for every pair of sequences that are to be aligned. These scores are presented in a table in the results. Pairwise scores are calculated as the number of identities in the best alignment divided by the number of residues compared (gap positions are excluded). Both of these scores arc initially calculated as percent identity scores and are converted to distances by dividing by 100 and subtracting from 1.0 to give number of differences per site. We do not correct for multiple substitutions in these initial distances. As the pairwise score is calculated independently of the matrix and gaps chosen, it will always be the same value for a particular pair of sequences.”

It should be noted that a coding'region is considered to be of or from an organism if it encodes a protein sequence substantially identical to that of the same protein purified from cells of the organism. In general, sequences are considered to be “substantially identical” if they share one or more characteristic sequences, and/or if they differ at no more than about 25% of residues. In some embodiments, substantially identical sequences differ at no more than about 10%, 9%, 8%, 7%, 6%, 5%, 4%, 3%, 2%, 1% of their positions, or less.

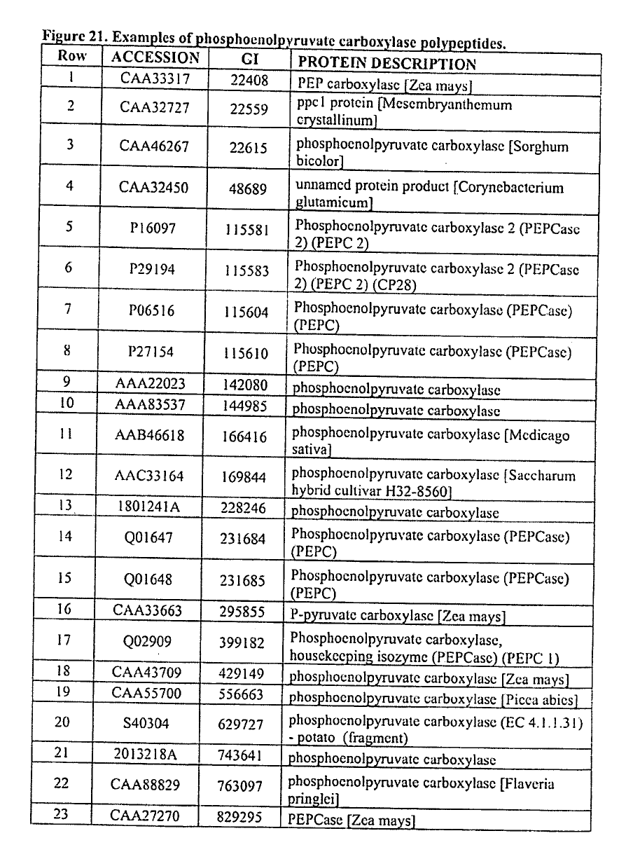

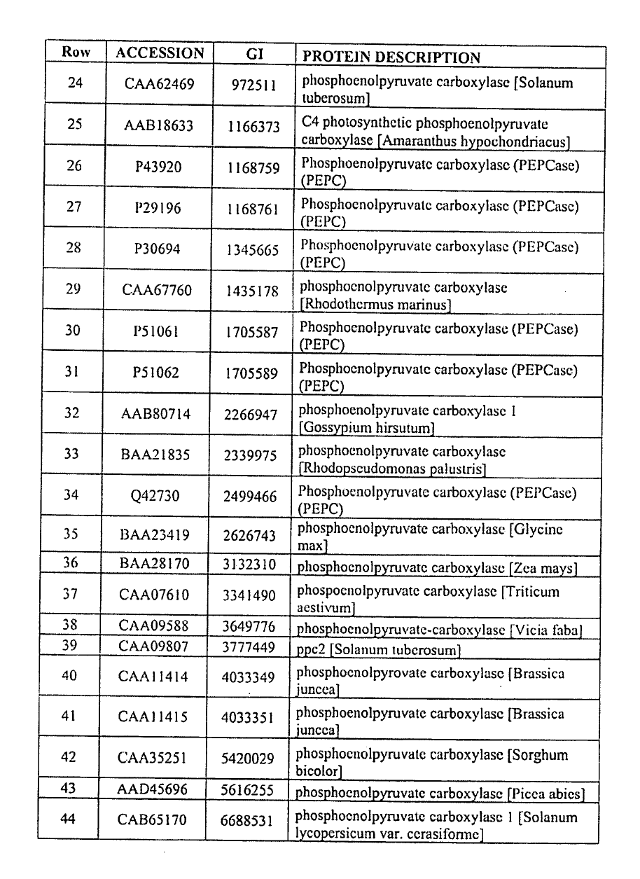

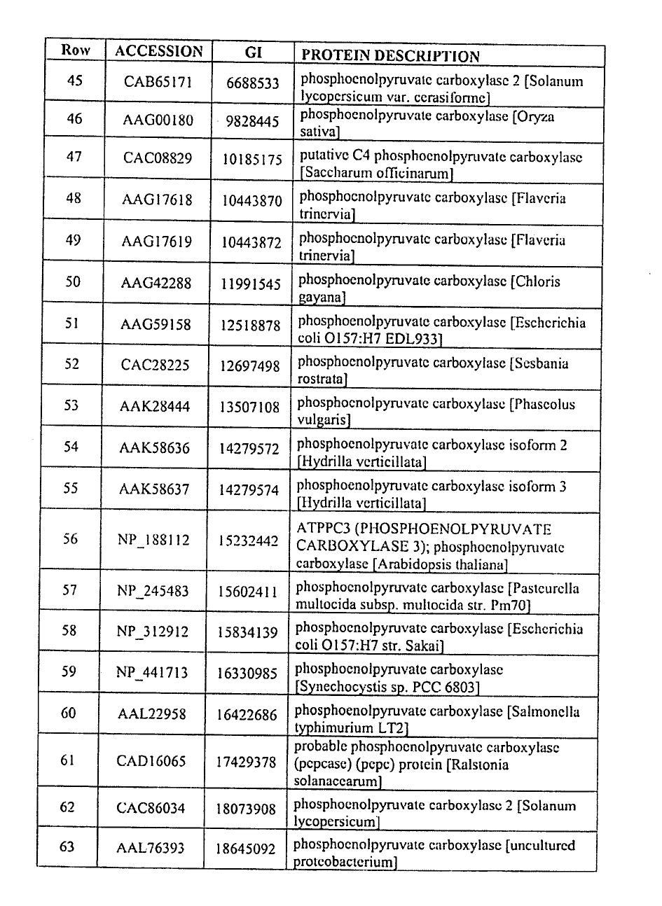

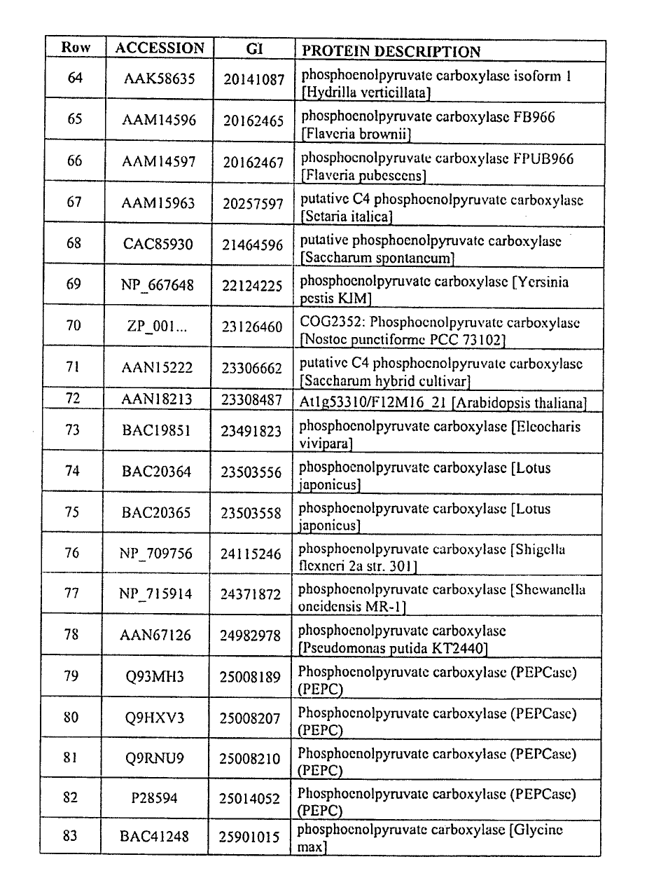

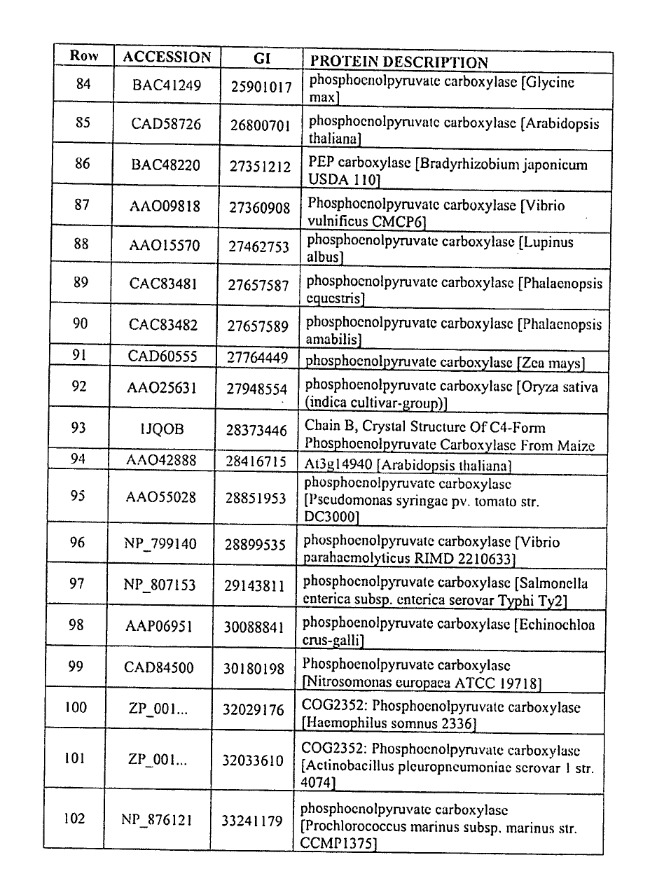

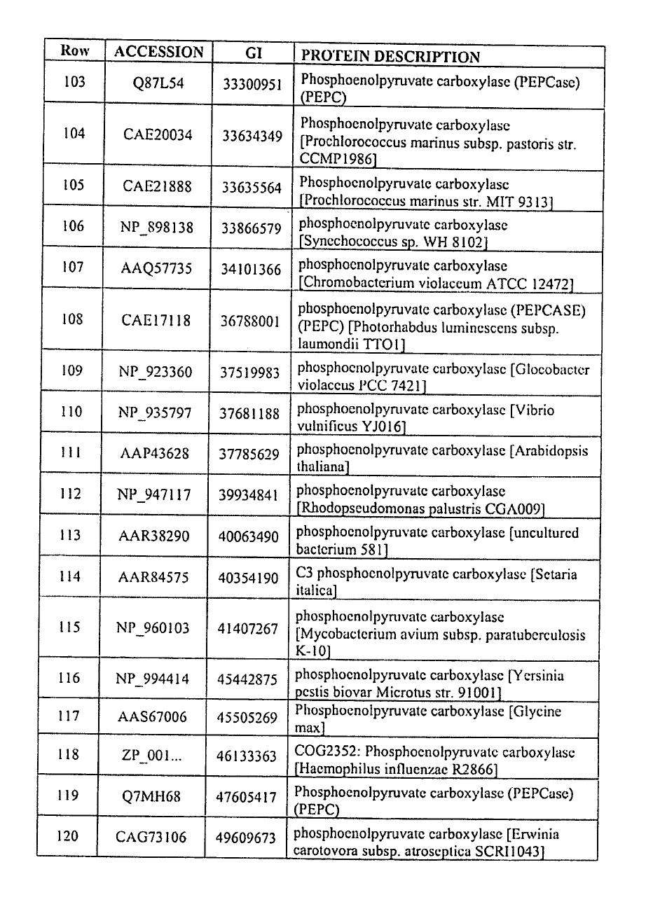

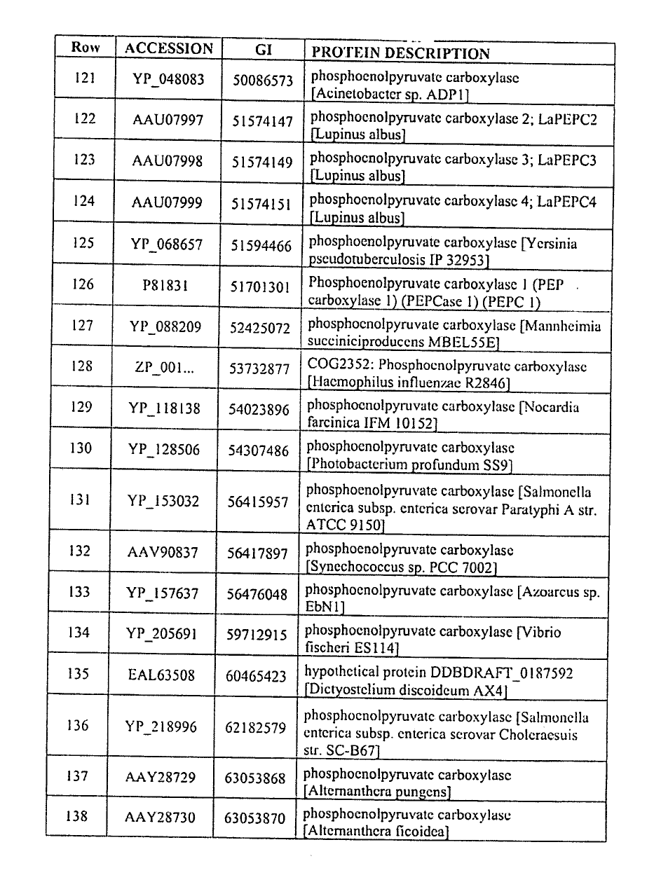

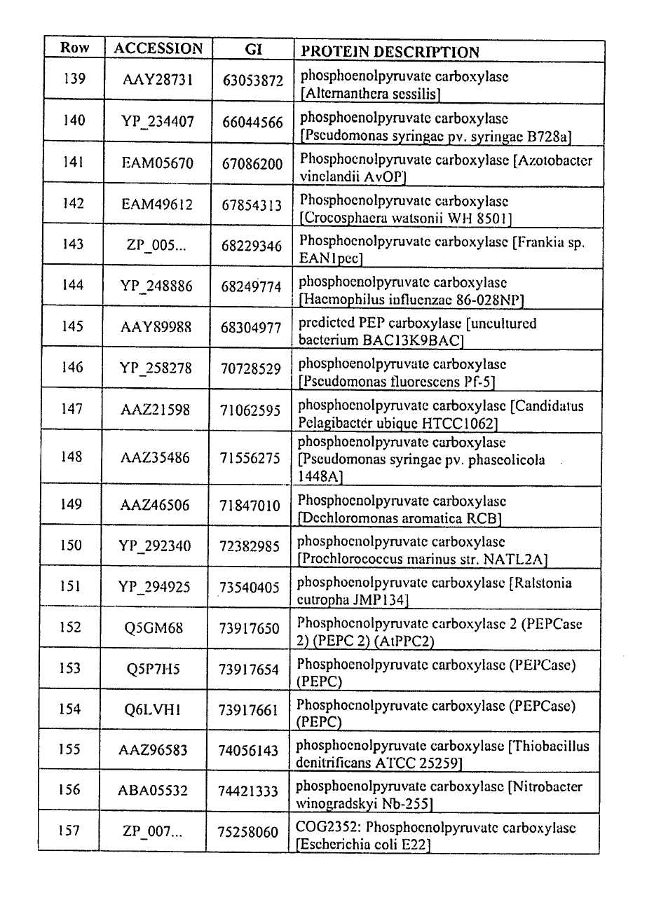

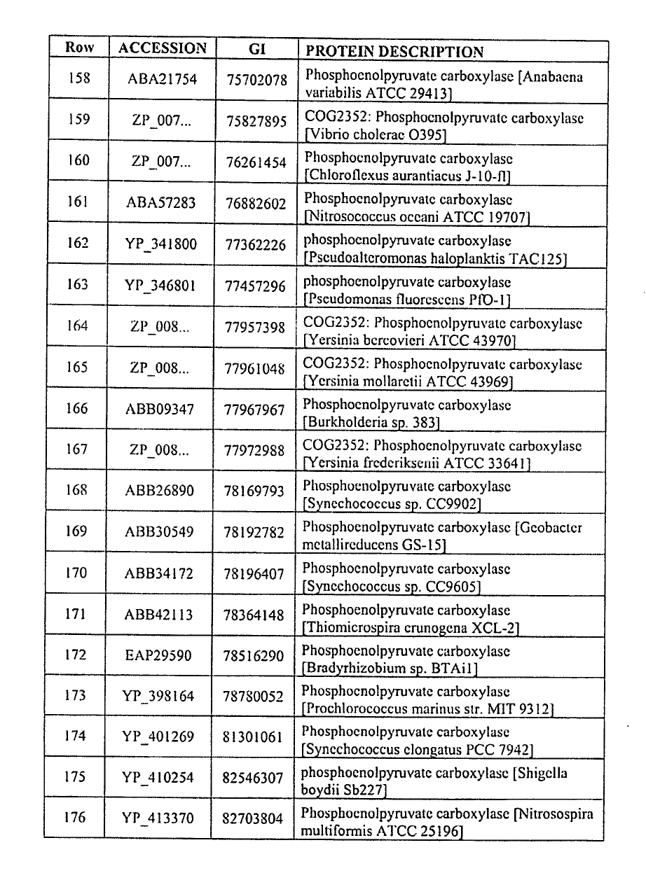

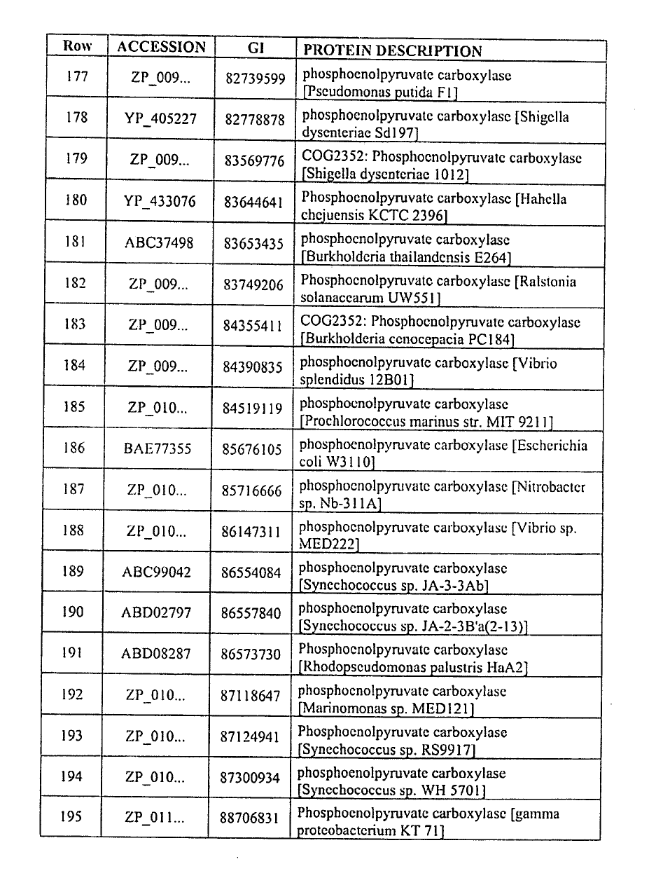

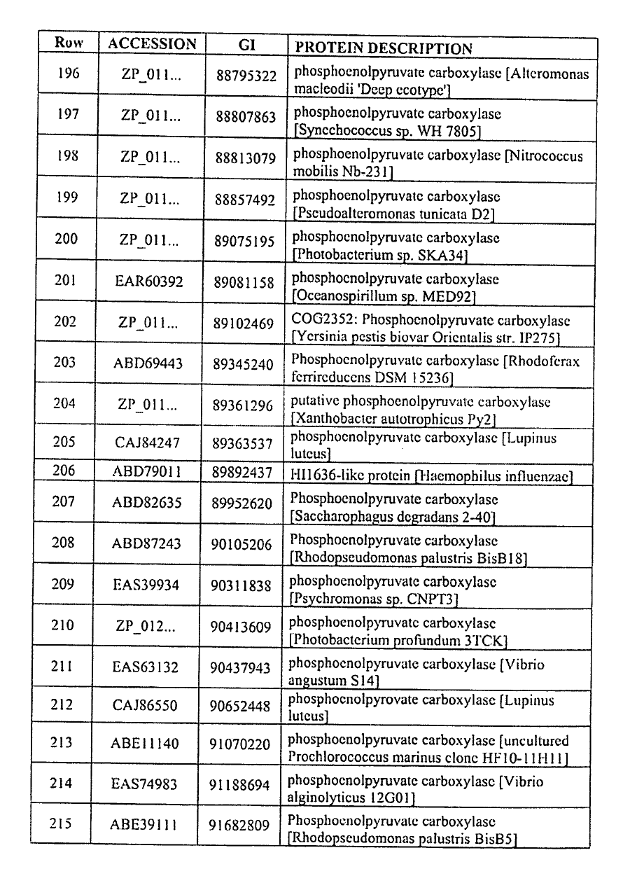

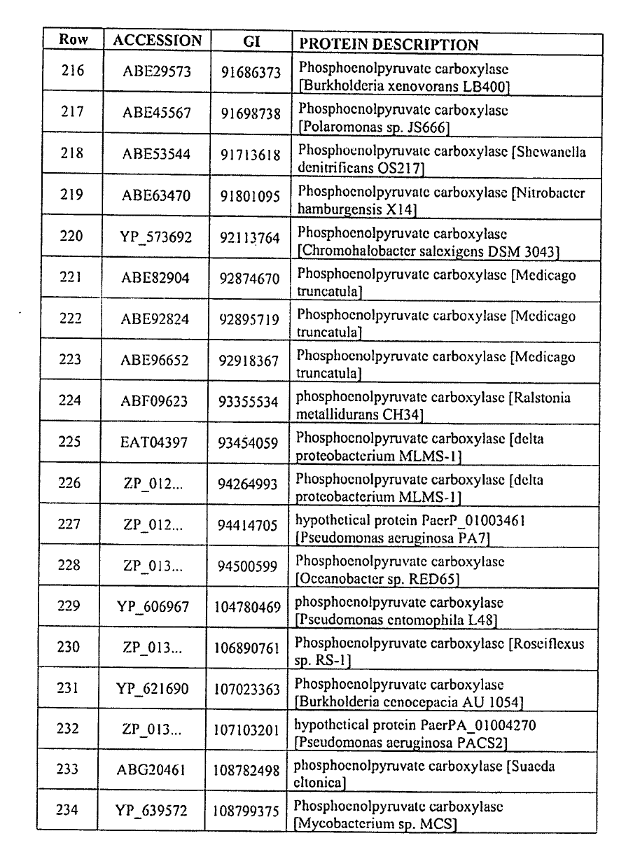

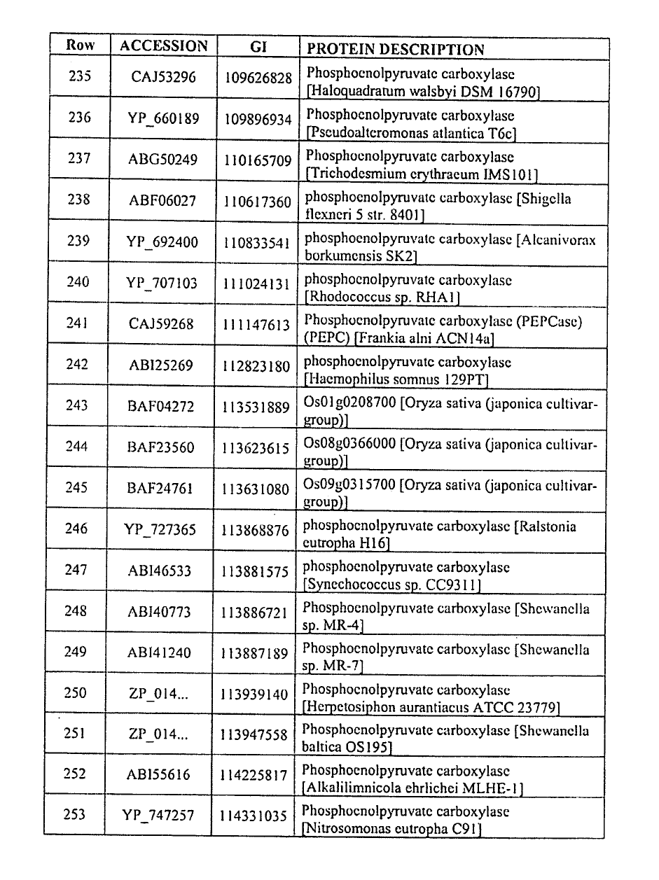

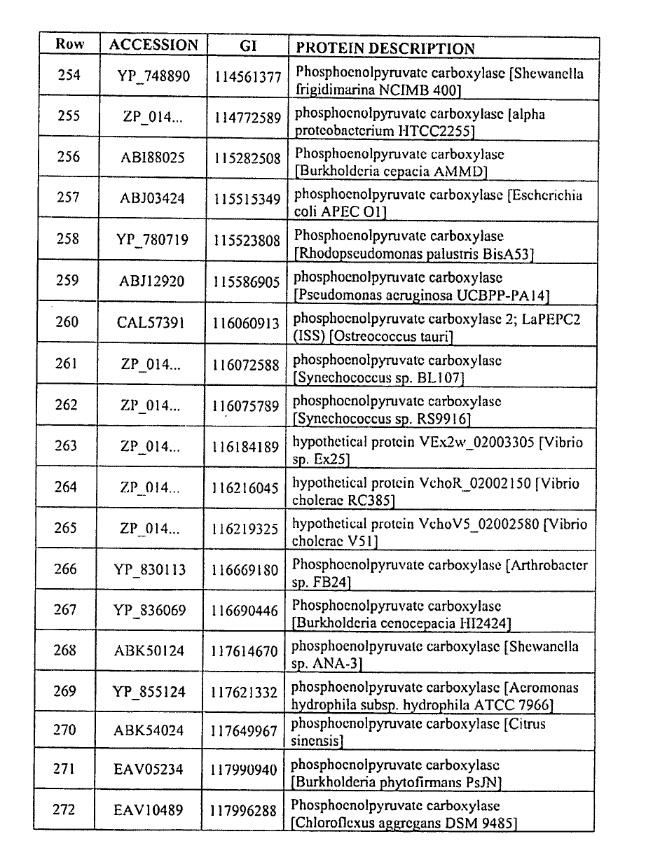

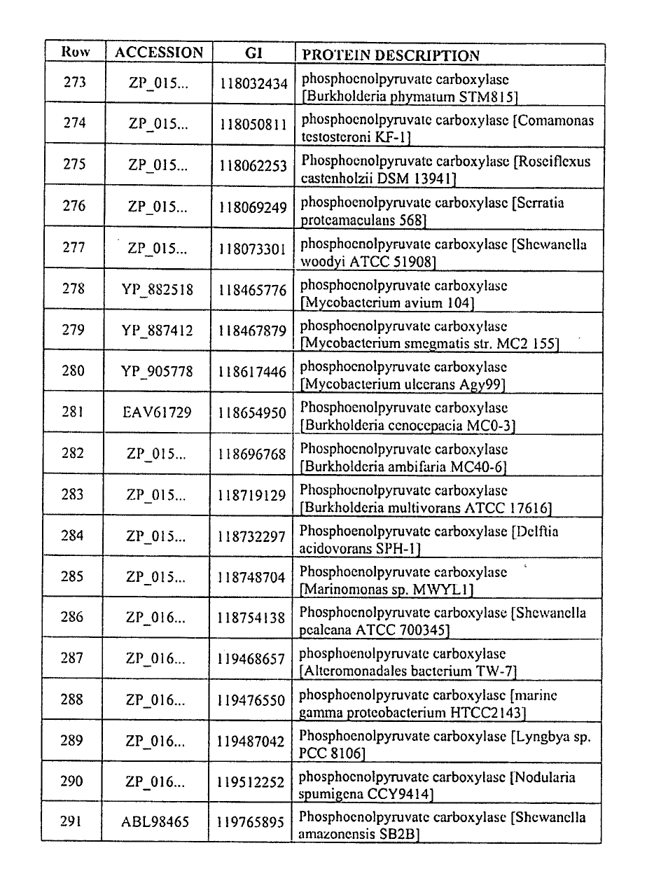

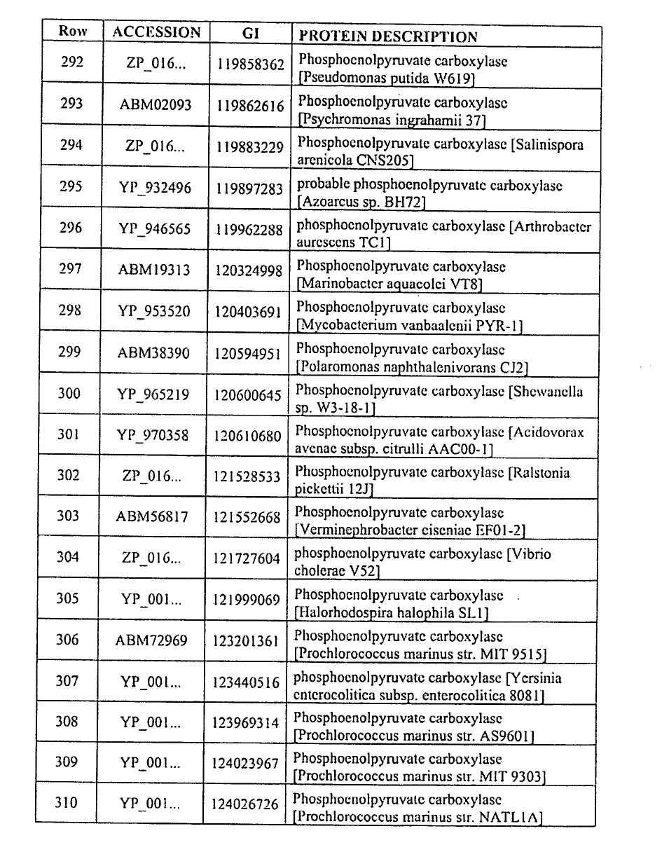

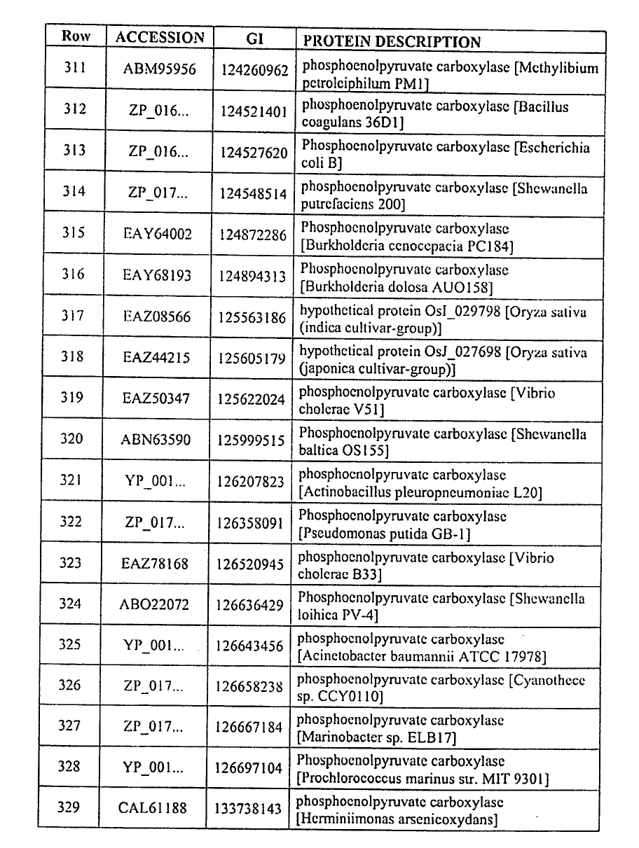

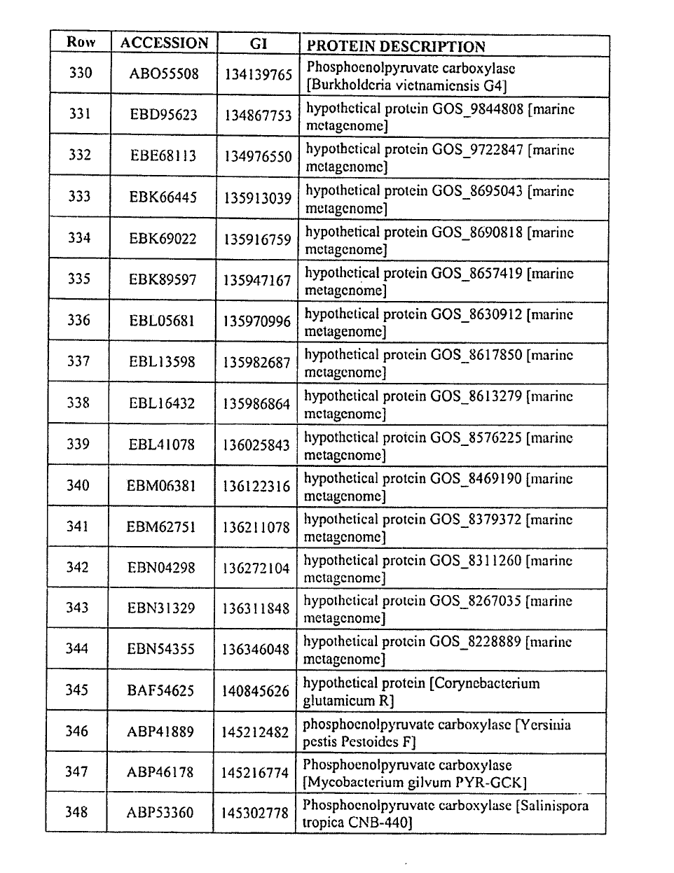

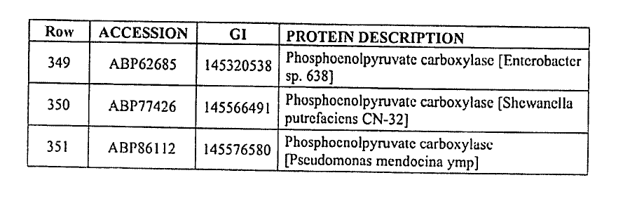

In one embodiment, the yeast can be transformed to increase the activity of a phosphoenolpyruvate carboxylase (PPC) polypeptide (EC 4.1.1.31), either as an alternative to or in addition to the PYC. A “phosphoenolpyruvate carboxylase (PPC) polypeptide” is a polypeptide catalyzes the addition of carbon dioxide to phosphoenolpyruvate (PEP) to form oxaloacetate (EC 4.1.1.31). E. coli PPC has been observed to be negatively regulated by downstream products including by malate. An enzyme need not be identified in the literature as a PPC at the time of filing of the present application to be within the definition of a PPC polypeptide. A PPC from any source organism may be used and the PPC may be wild type or modified from wild type. In some embodiments, the PPC polypeptide is less sensitive to inhibition by one or more of malate, aspartate, and oxaloacetate. E. coli PPC has been observed to be inhibited by malate. In certain embodiments, the PPC polypeptide has the amino acid sequence of SEQ ID NO:7 or a PPC enzyme in FIG. 18 or has at least 99%, 98%, 97%, 96%, 95%, 94%, 93%, 92%, 91%, 90%, 85%, 80%, 75% identity to a PPC in FIG. 18. In another embodiment, the PPC polypeptide is a polypeptide represented by the Genbank GI numbers in FIG. 21 or has at least 99%, 98%, 97%, 96%, 95%, 94%, 93%, 92%, 91%, 90%, 85%, 80%, 75% identity to a polypeptide represented by the Genbank GI numbers in FIG. 21. In some embodiments the PPC polypeptide is an E. coli PPC polypeptide with the lysine at position 620 substituted with a serine and/or the lysine at position 773 substituted with a glycine.

A malate dehydrogenase (MDH) polypeptide for use in accordance with the present disclosure is any enzyme capable of catalyzing the introconversion of oxaloacetate to malate (using NAD(P)+) and vice versa (EC 1.1.1.37). Malate dehydrogenase polypeptides can be localized to the mitochondria or to the cystosol. In some embodiments, the MDH is active in the cytosol. In some embodiments, the MDH polypeptide retains activity (i.e. units of MDH activity) in the presence of glucose. In some embodiments, activity of the MDH polypeptide in the absence of glucose is at least least 20%, 30%, 40%, 50%, 60%, 70%, 80%, 90%, 95%, 96%, 97%, 98%, 99%, 99%, or 100% of that observed under otherwise identical activity in the presence of glucose. Such an MDH polypeptide is considered “not inactivated” in the presence of glucose. An enzyme need not be identified in the literature as a malate dehydrogenase at the time of filing of the present'application to be within the definition of an MDH polypeptide. It should be noted that the terms “malate” and “malic acid” may be used interchangeably herein except in contexts where one particular ionic species is indicated. Similarly, the terms, “succinate” and “succinic acid” may be used interchangeably herein except in contexts where one particular ionic species is indicated

A MDH polypeptide from any source organism may be used in accordance with the present disclosure, and the MDH may be wild type or modified from wild type. In one embodiment, the MDH can be S. cerevisiae MDH1 or S. cerevisiae MDH3. Wild type S. cerevisiae MDH2 is active in the cytosol but is inactivated in the presence of glucose. In one embodiment, the MDH can be a modified S. cerevisiae MDH2 modified (by genetic engineering, posttranslational modification, or any other technique known in the art) to be active in the cytosol and not inactivated in the presence of glucose.

In one embodiment, a MDH polypeptide for use in accordance with the present disclosure contains a signaling sequence or sequences capable of targeting the MDH polypeptide to the cytosol of the yeast, or the MDH polypeptide lacks a signaling sequence or sequences capable of targeting the MDH polypeptide to an intracellular region of the yeast other than the cytosol. In one embodiment, the MDH polypeptide can be S. cerevisiae MDH3ΔSKL, in which the region encoding the MDH polypeptide has been altered to delete the carboxy-terminal SKL residues of wild type S. cerevisiae MDH3, which normally target the MDH3 to the peroxisome.

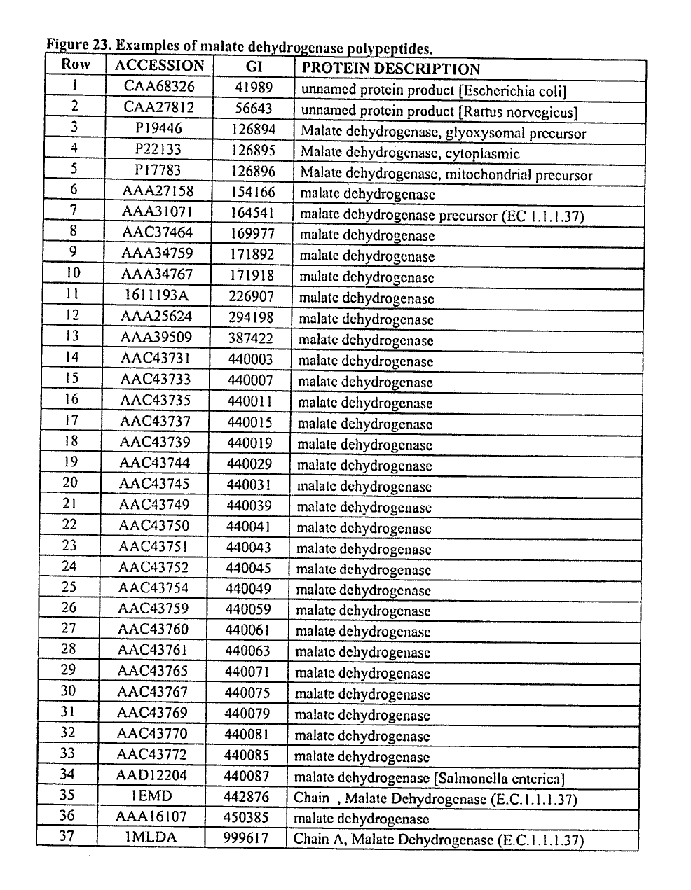

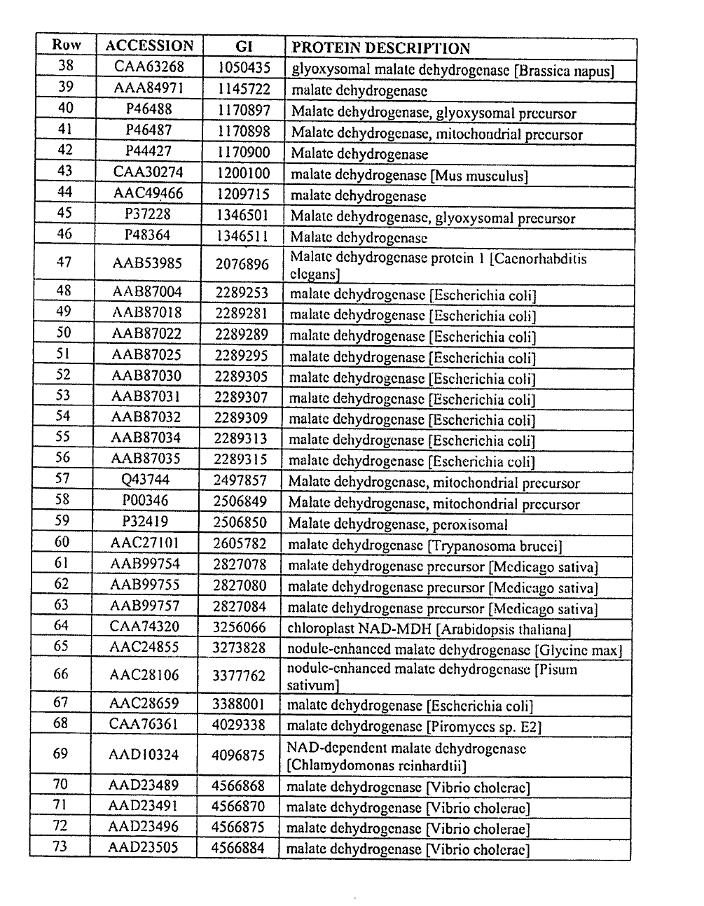

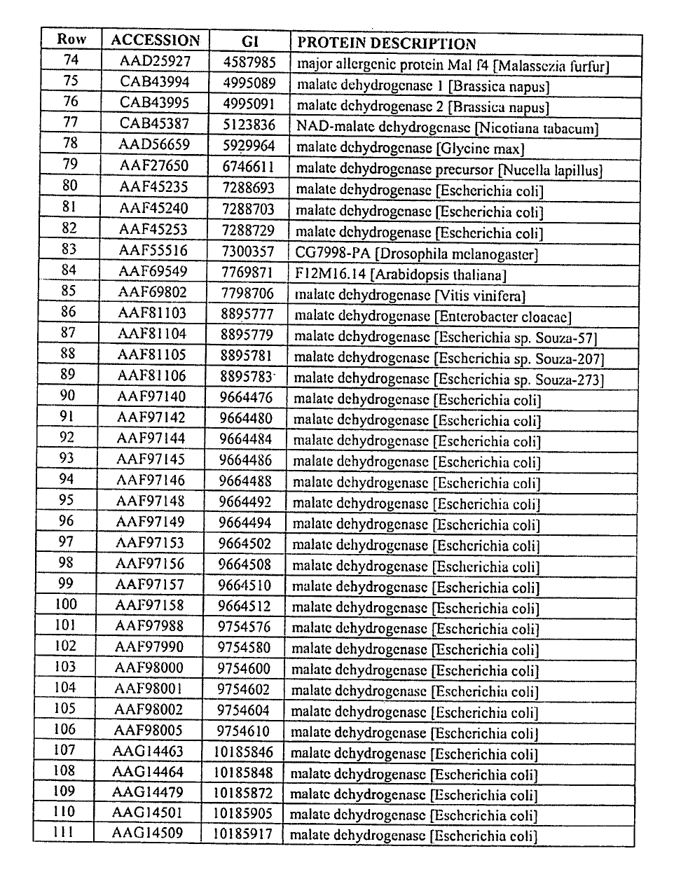

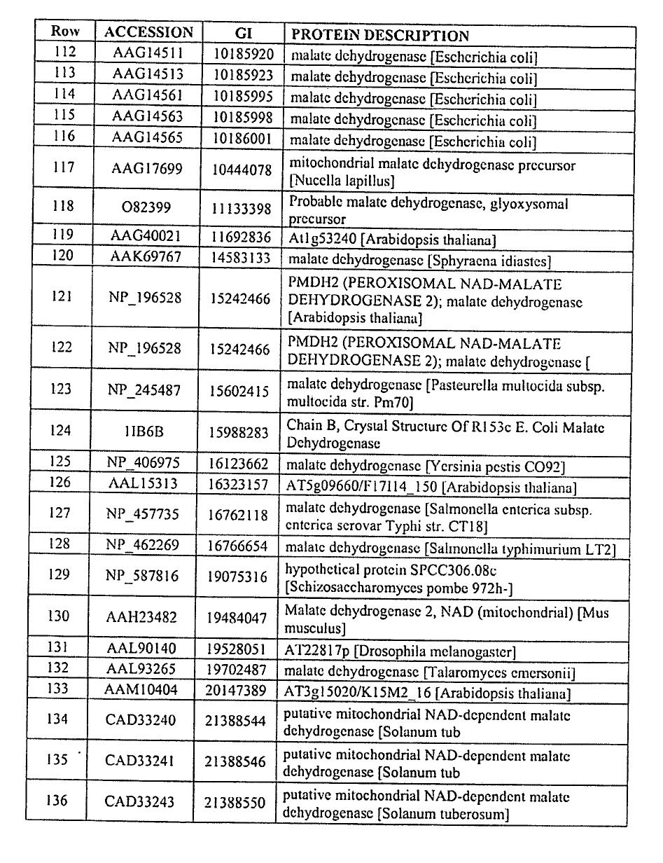

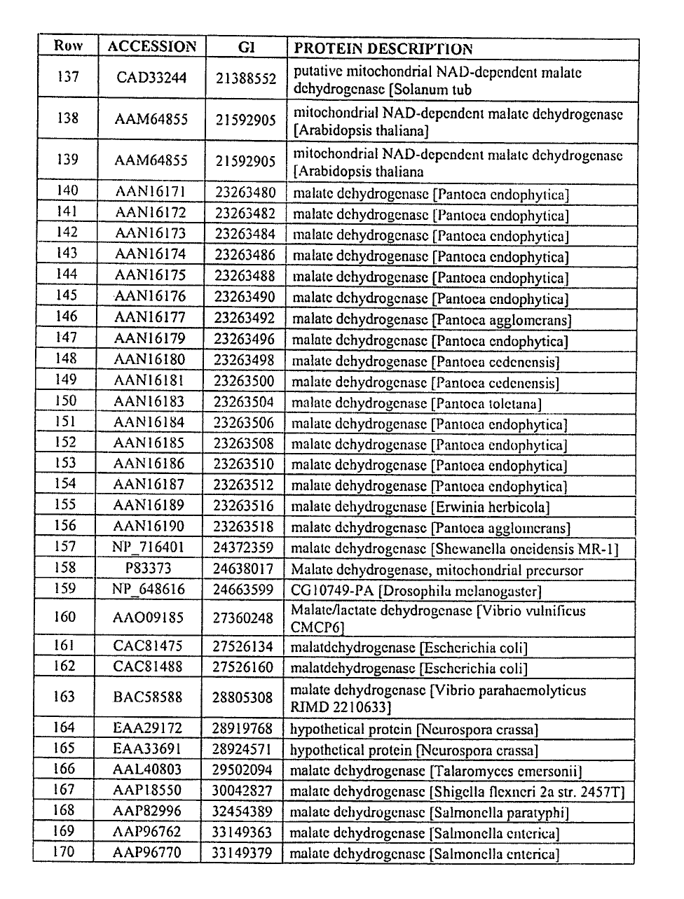

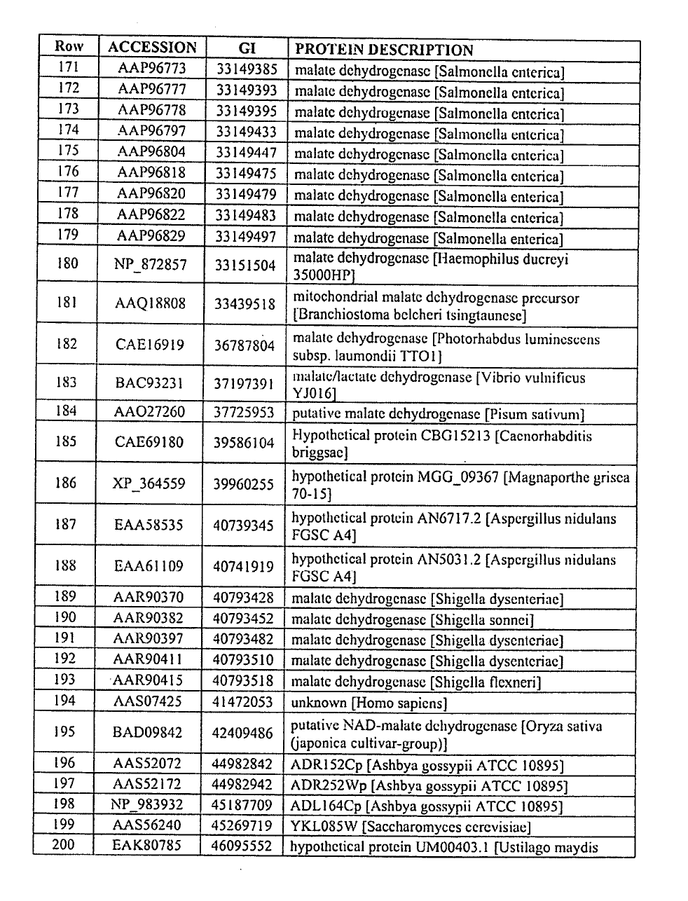

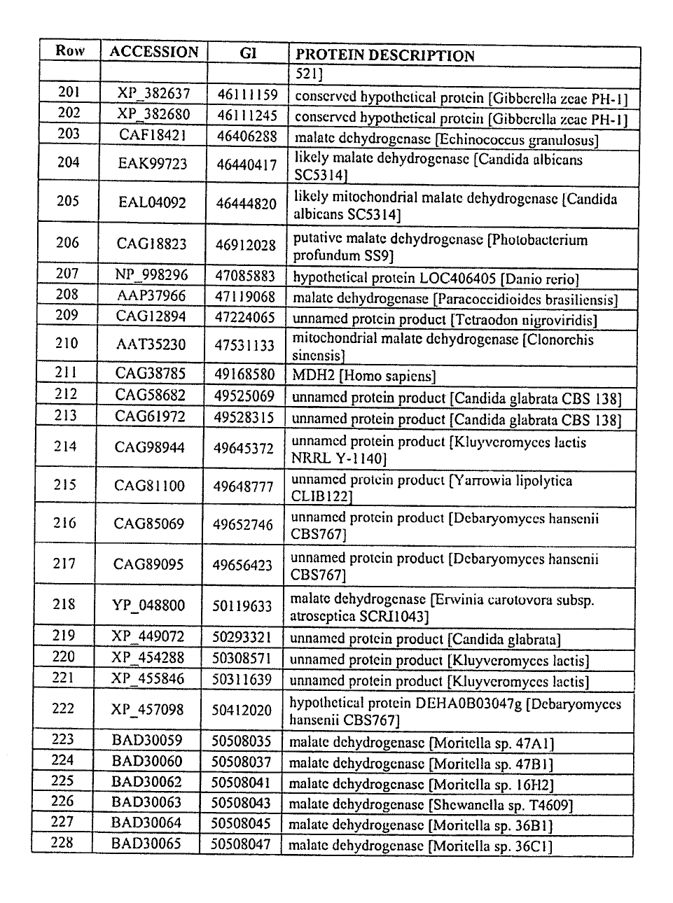

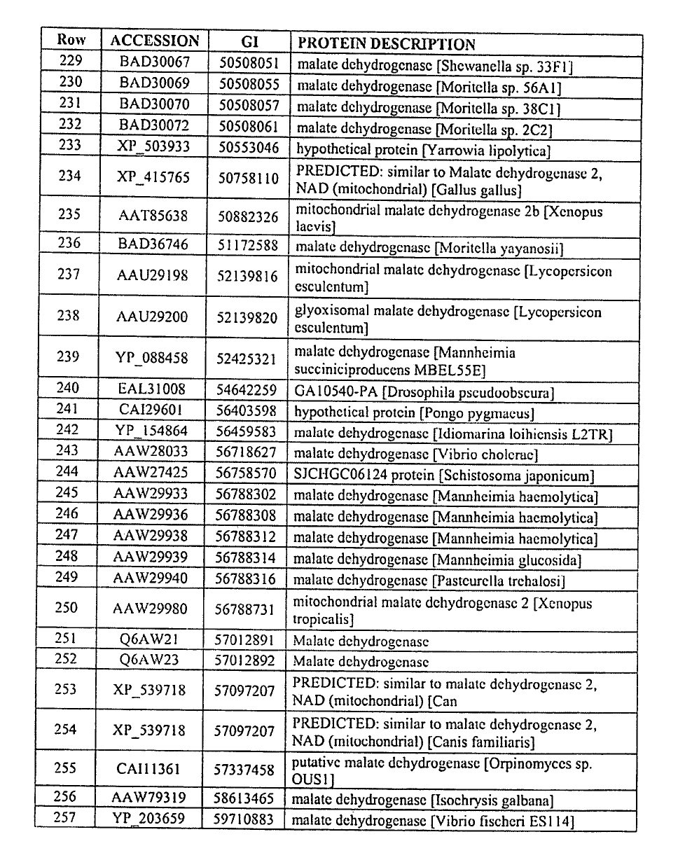

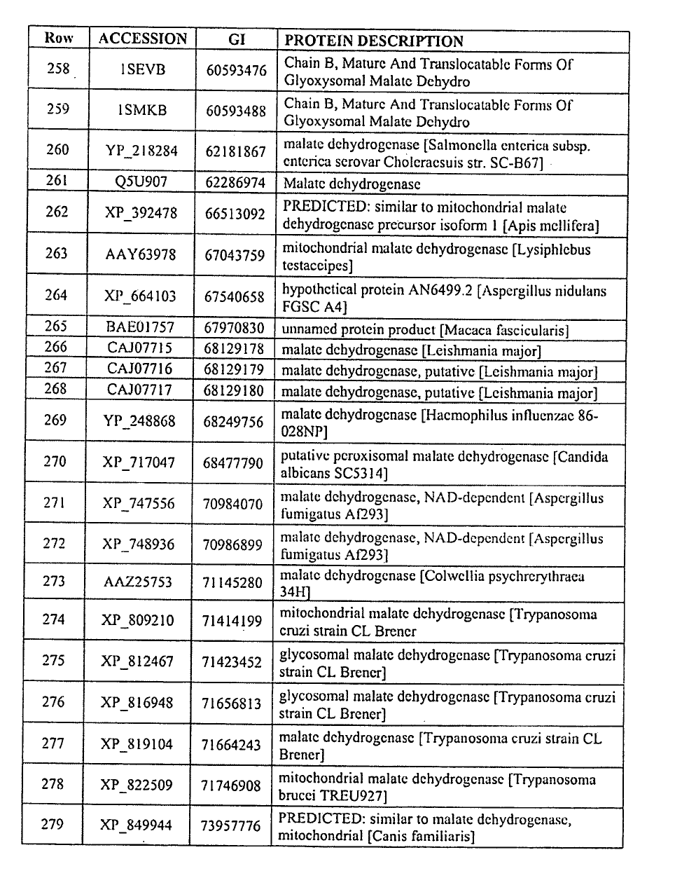

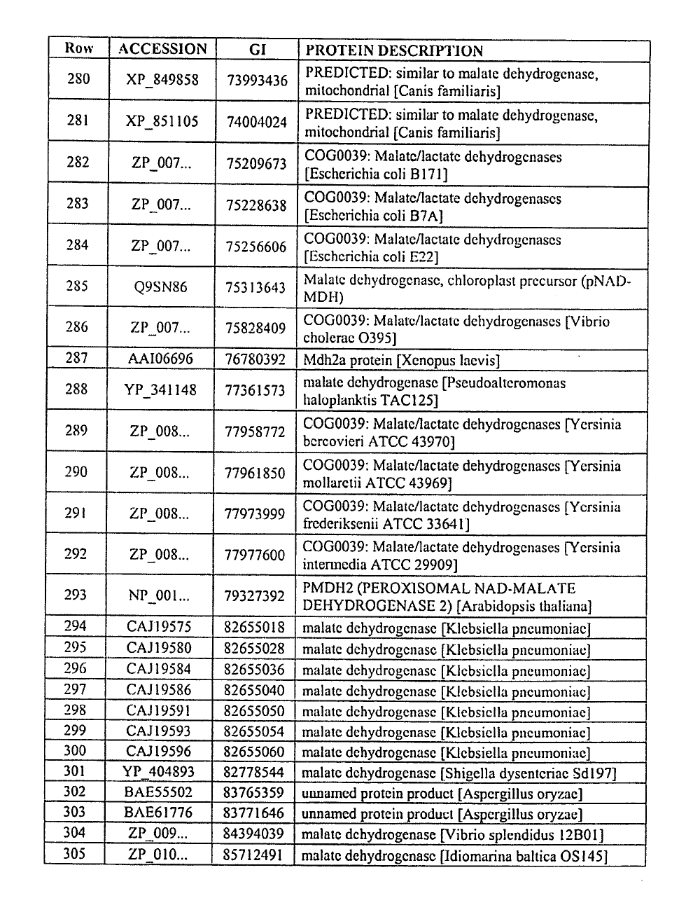

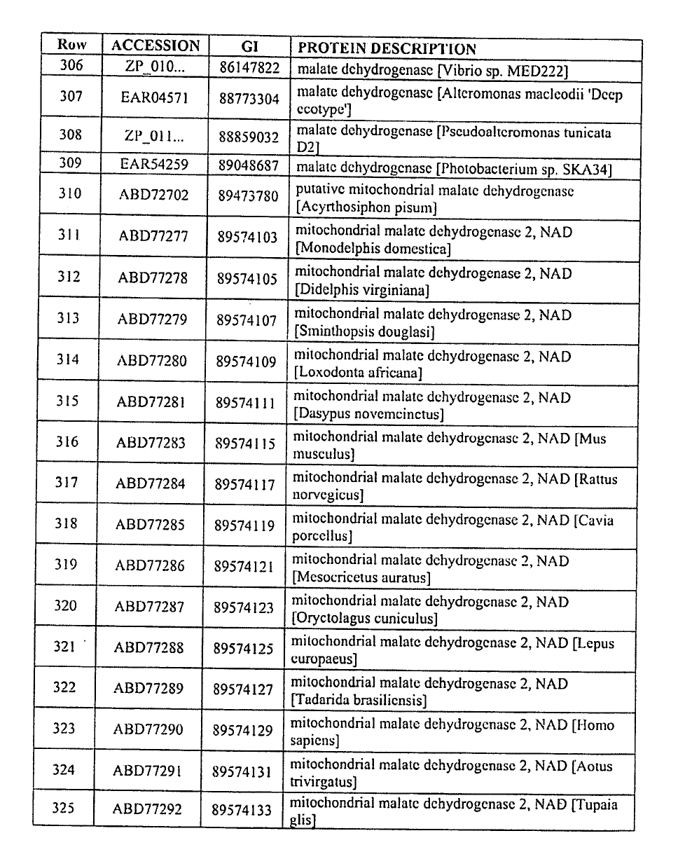

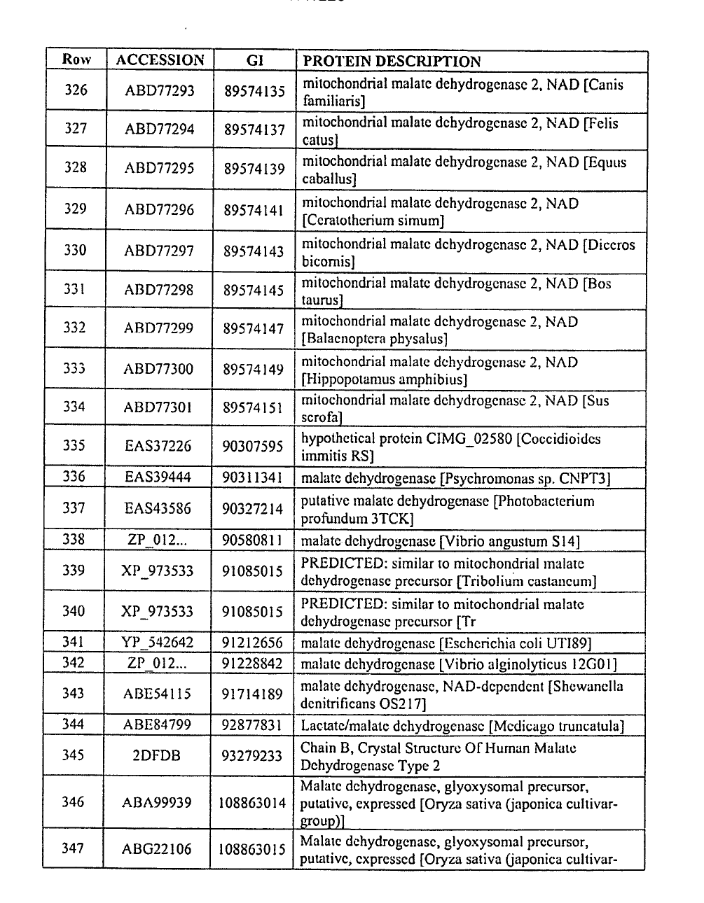

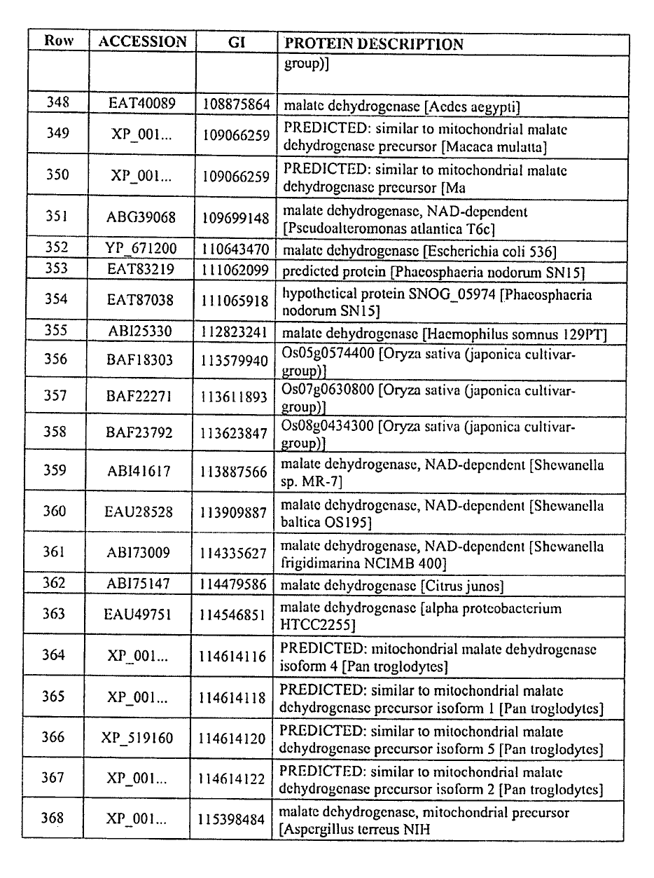

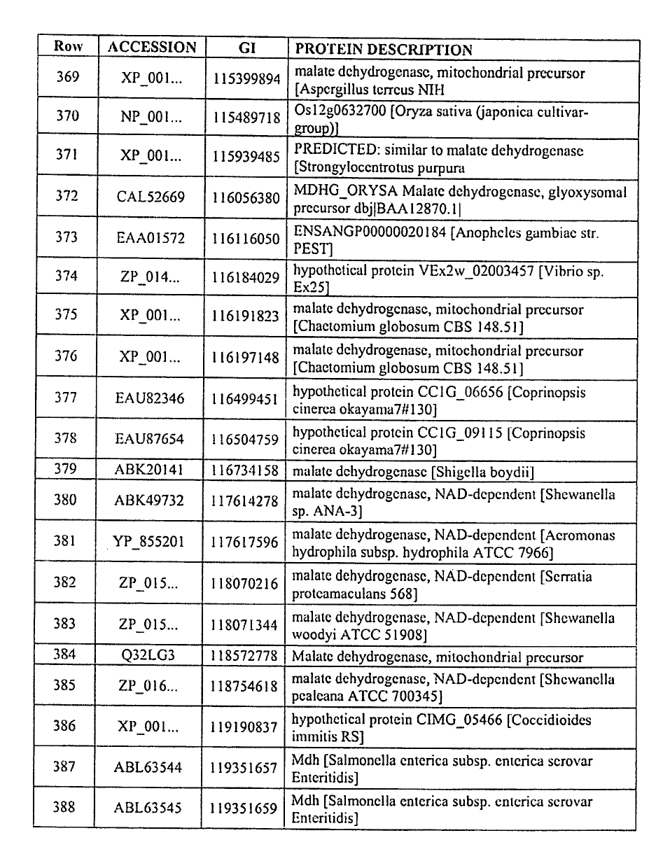

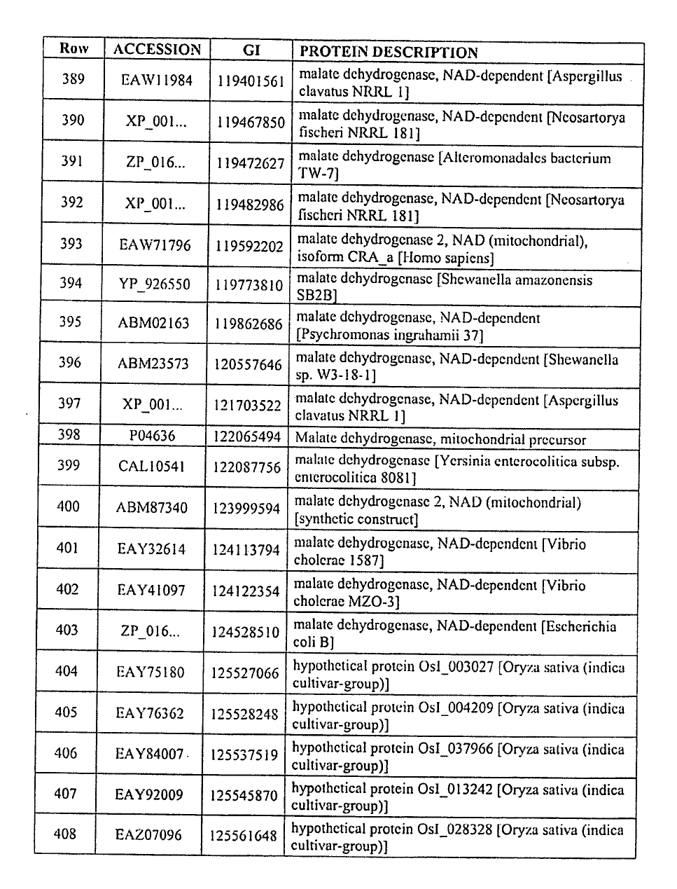

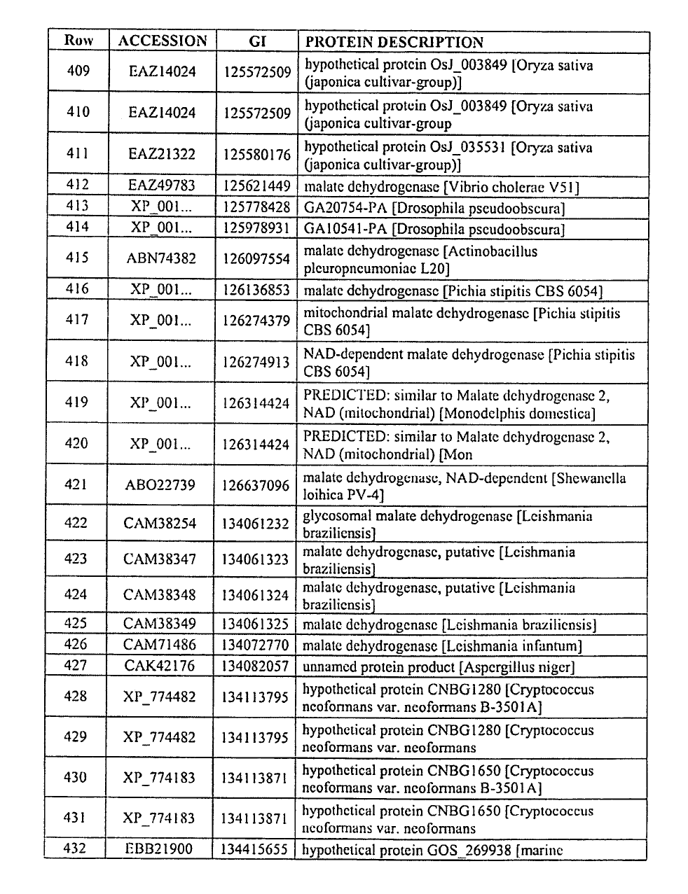

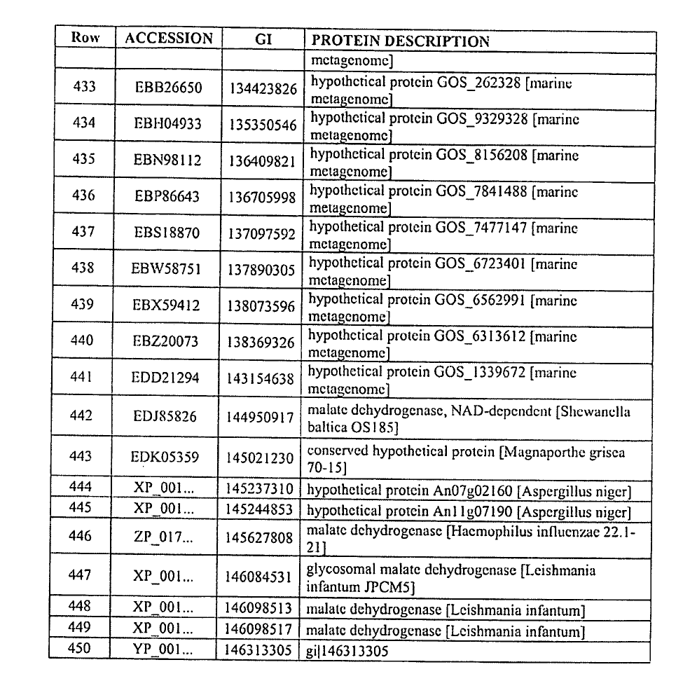

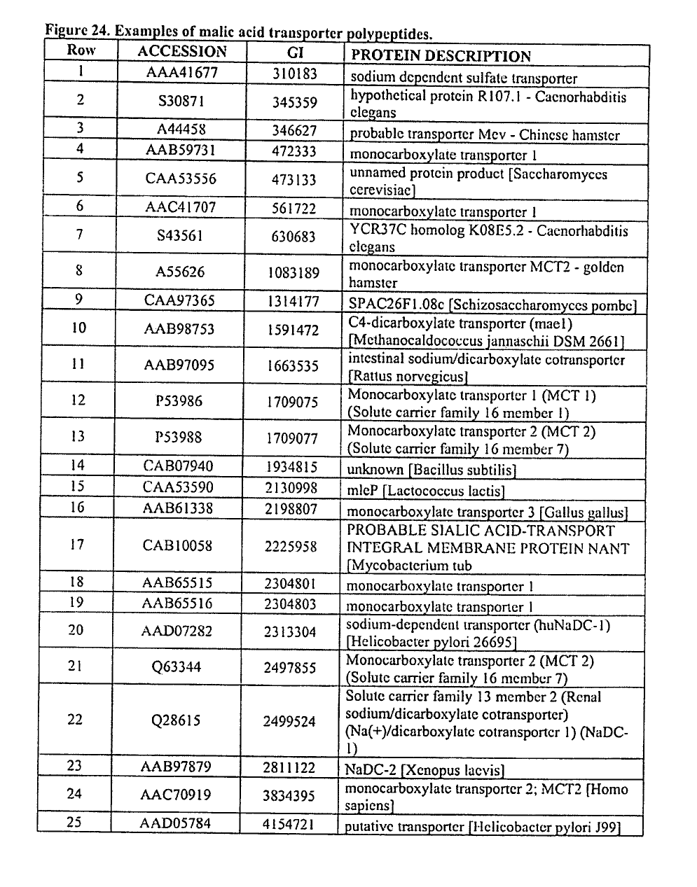

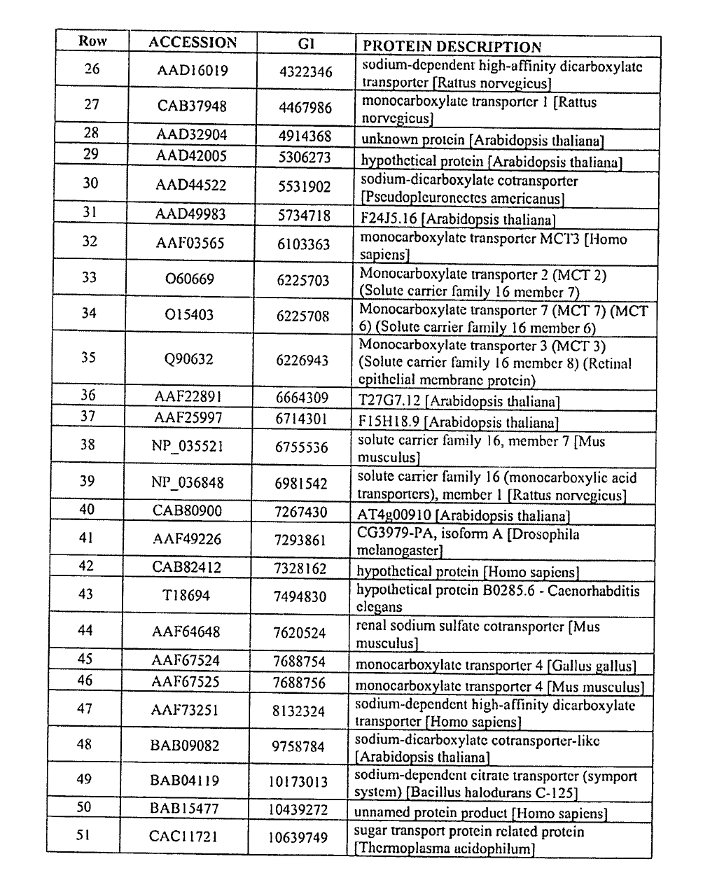

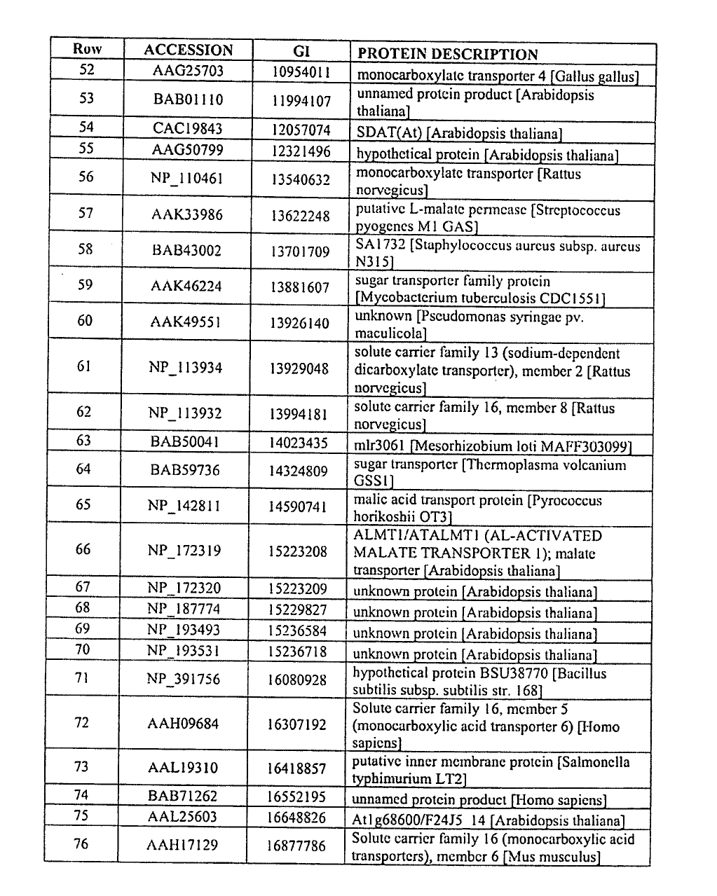

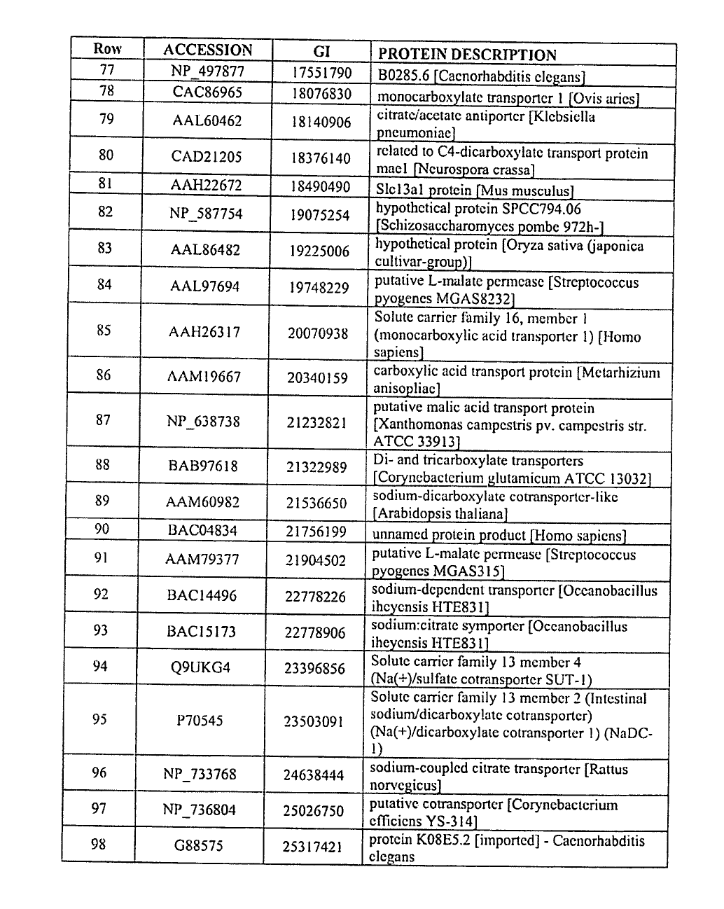

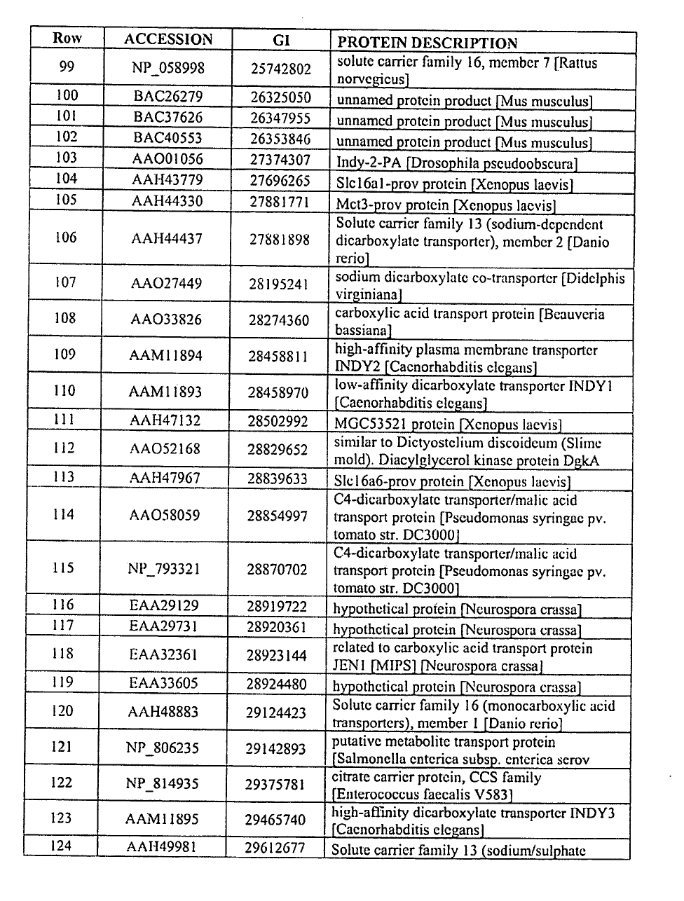

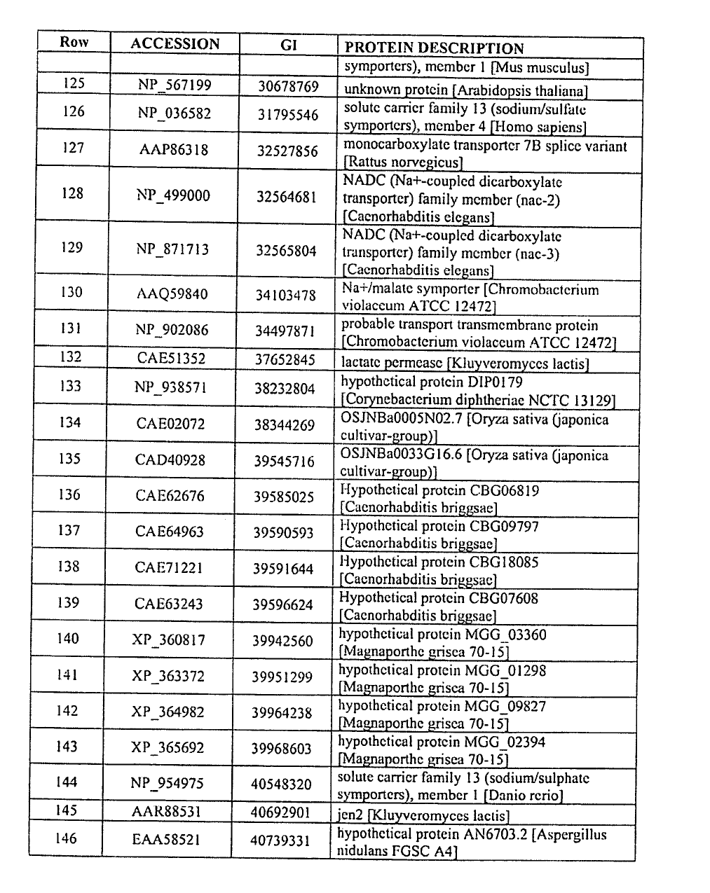

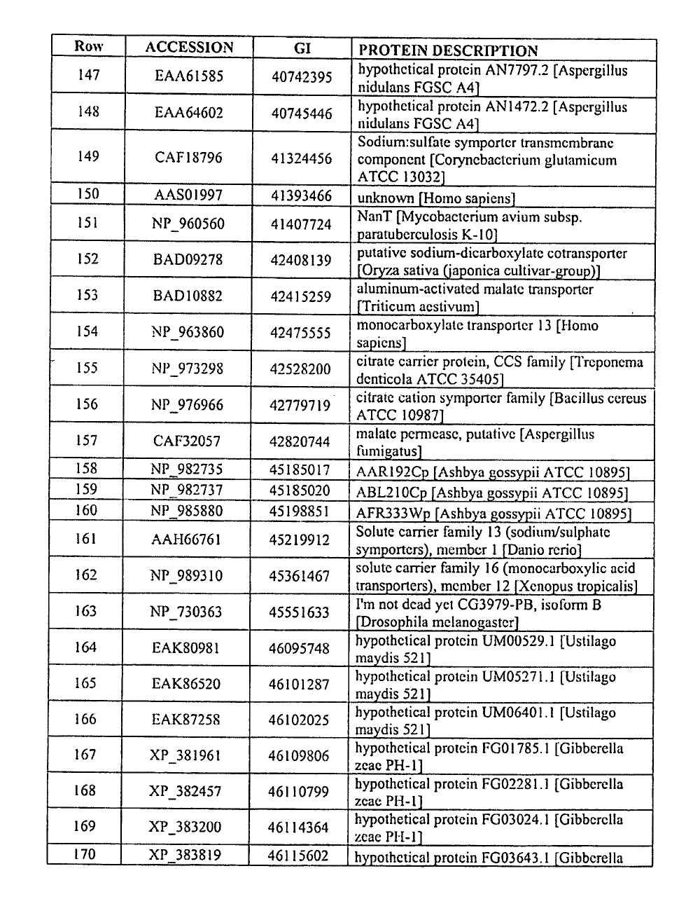

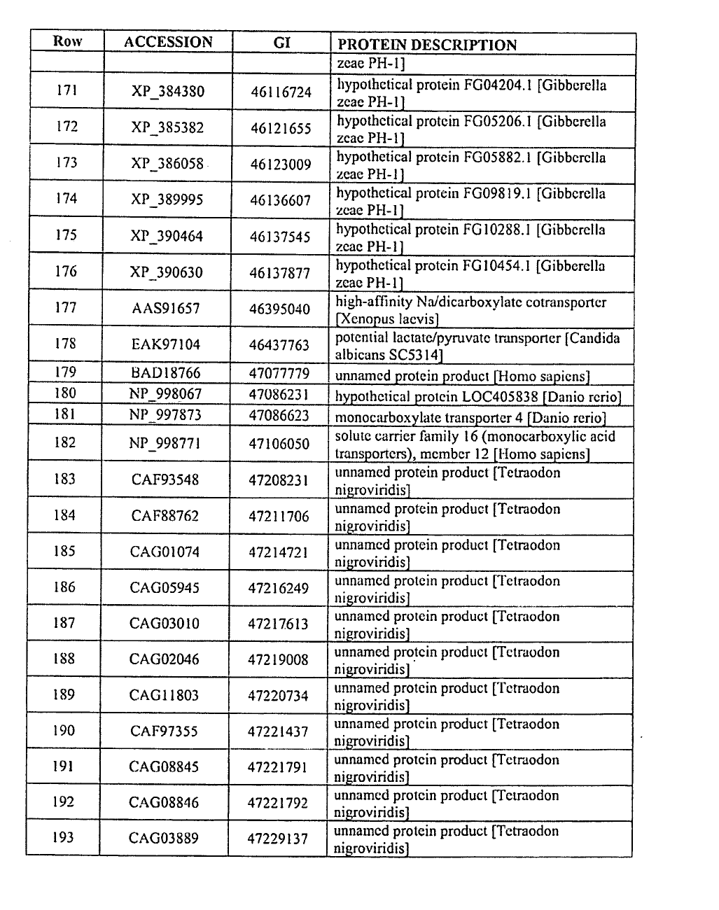

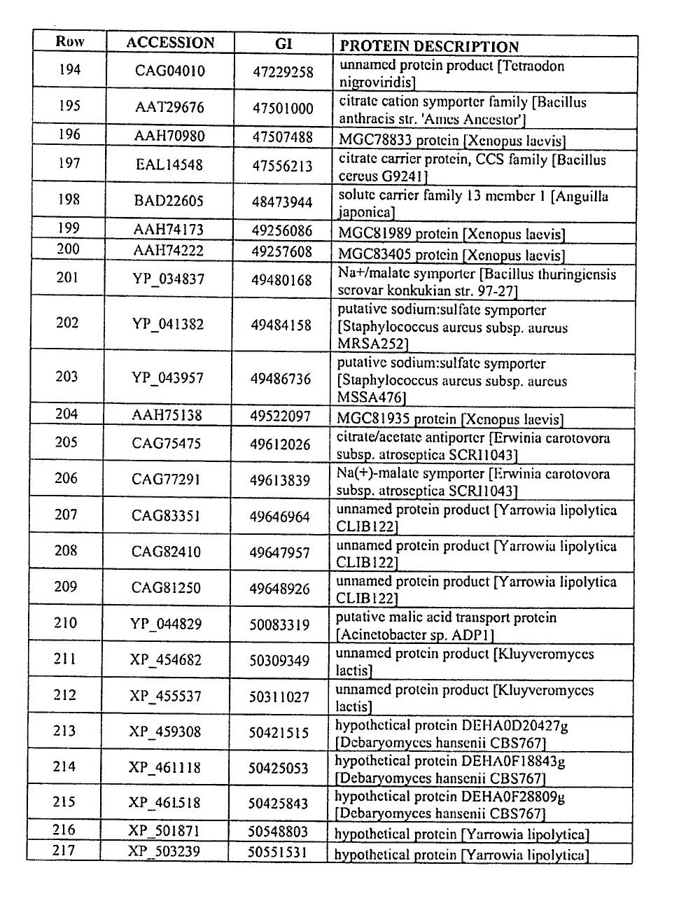

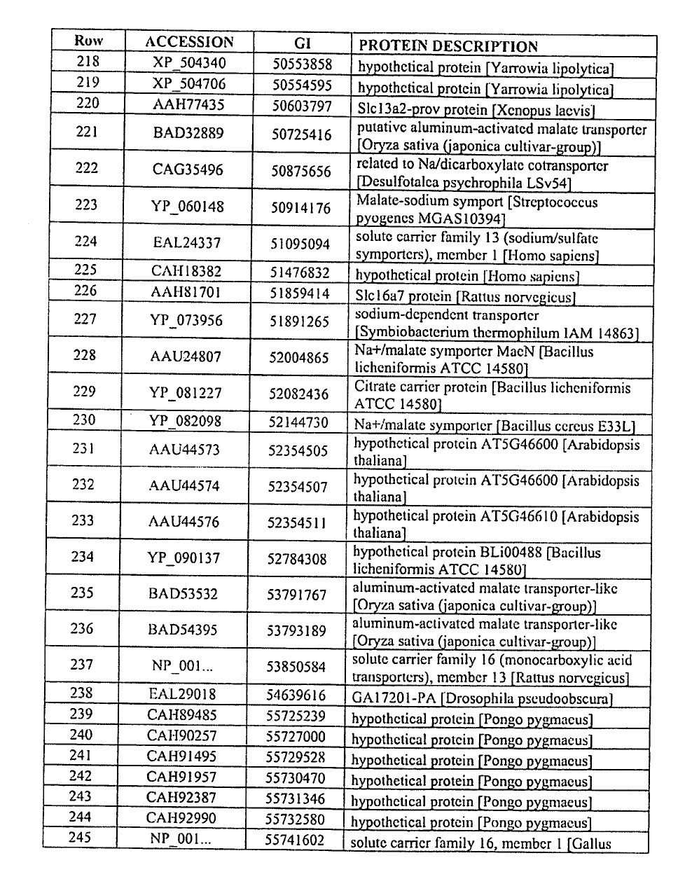

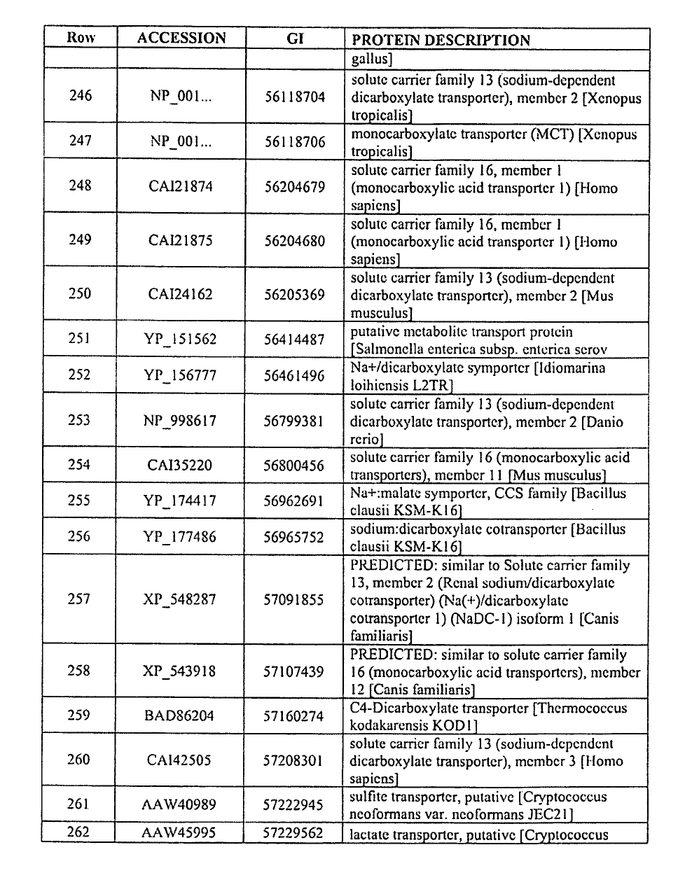

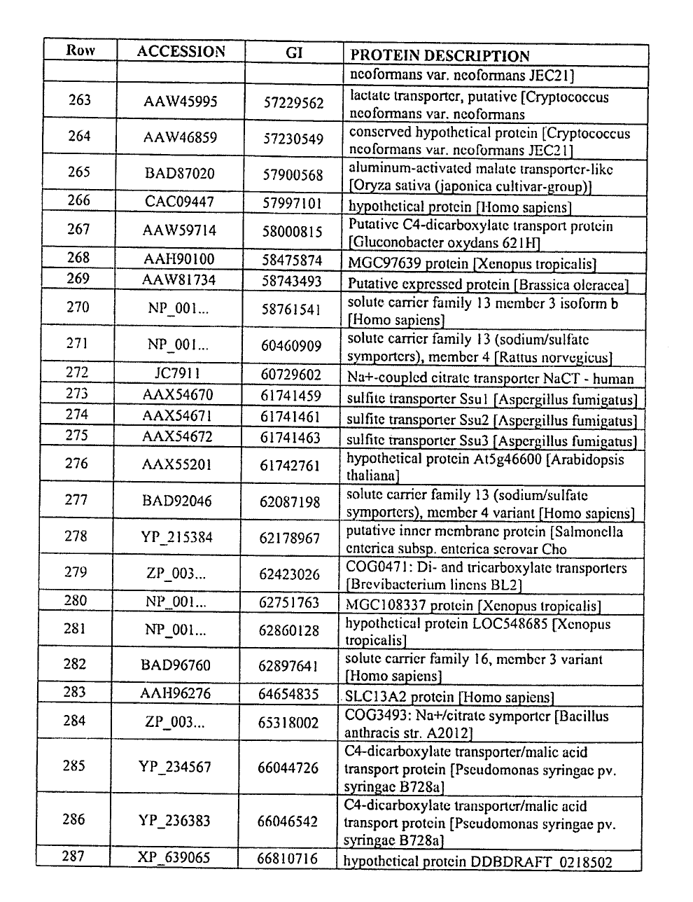

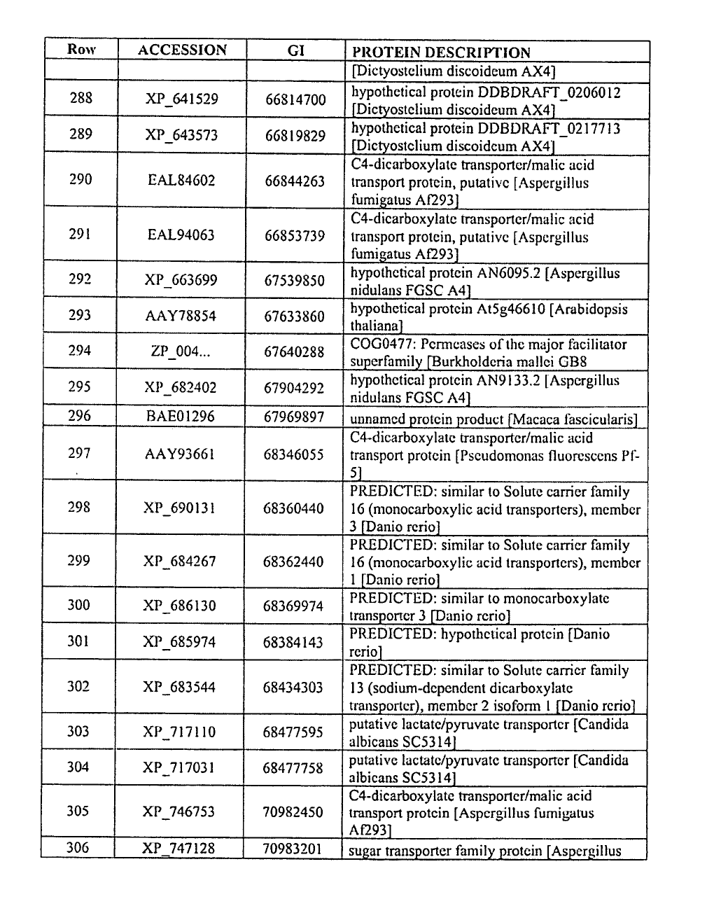

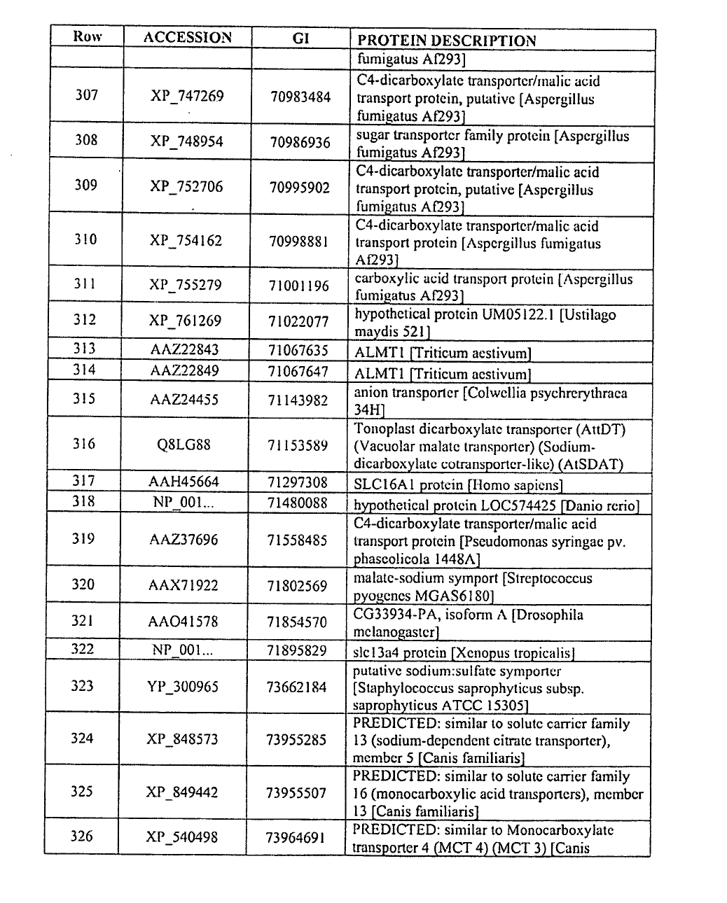

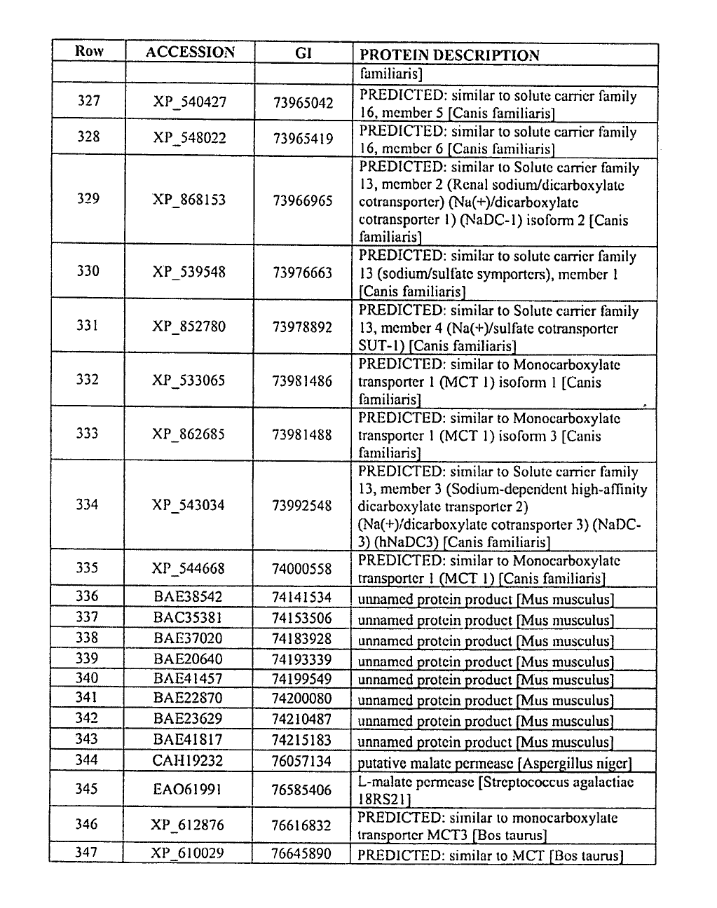

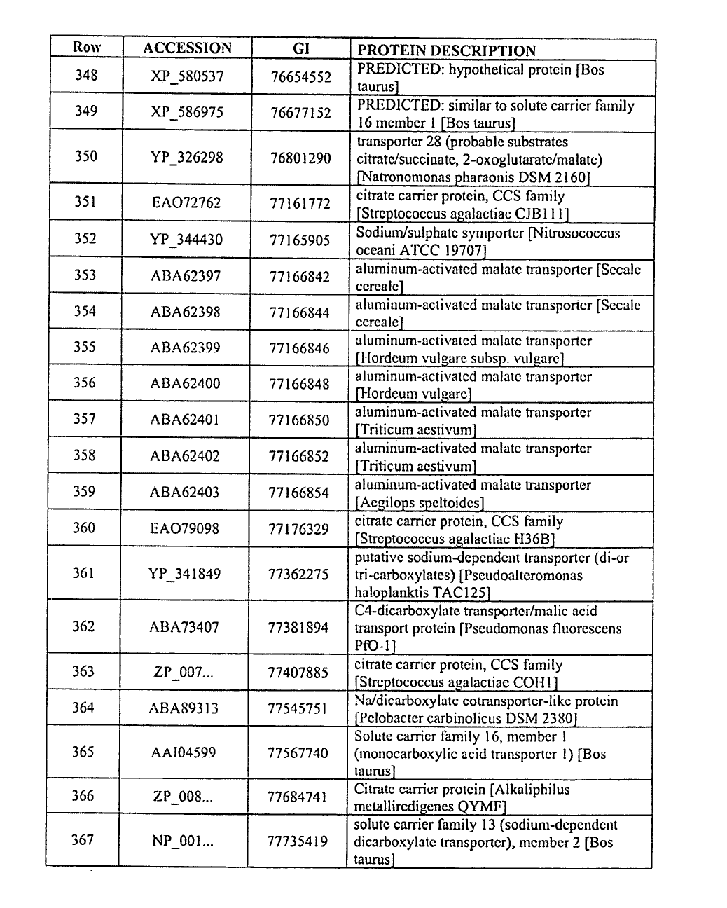

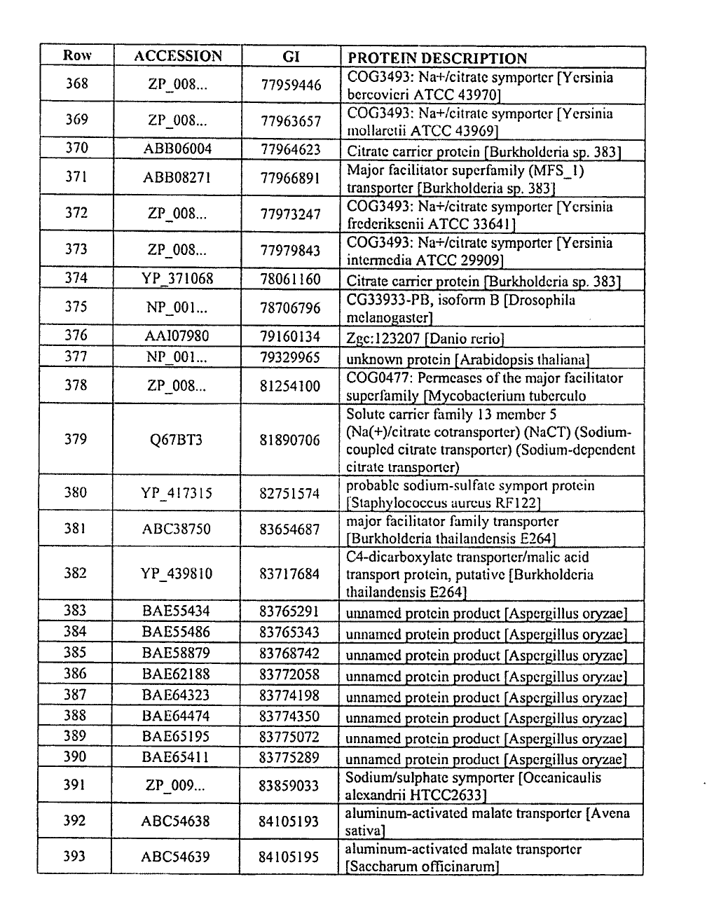

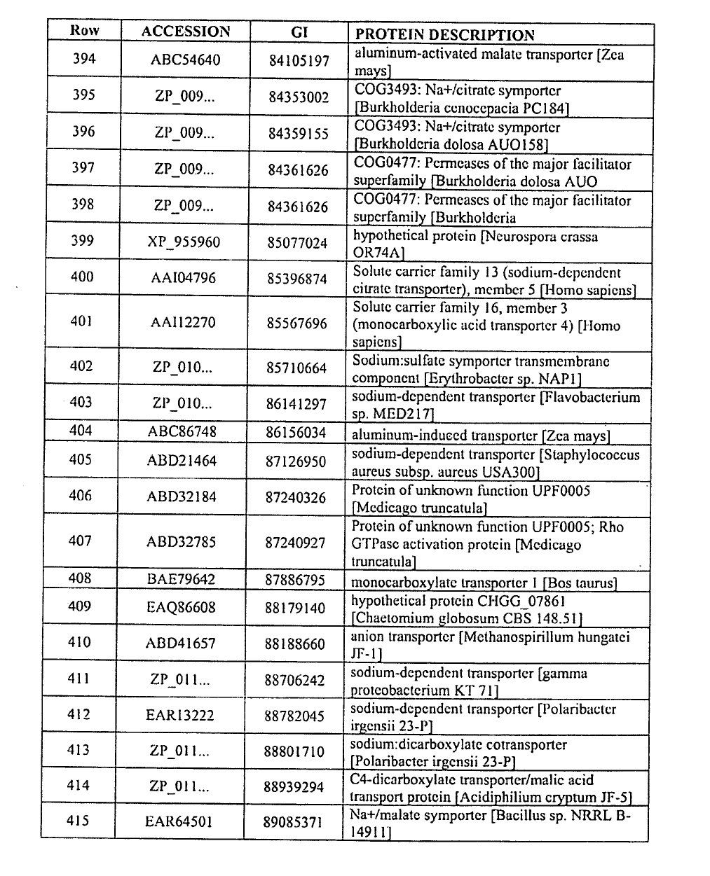

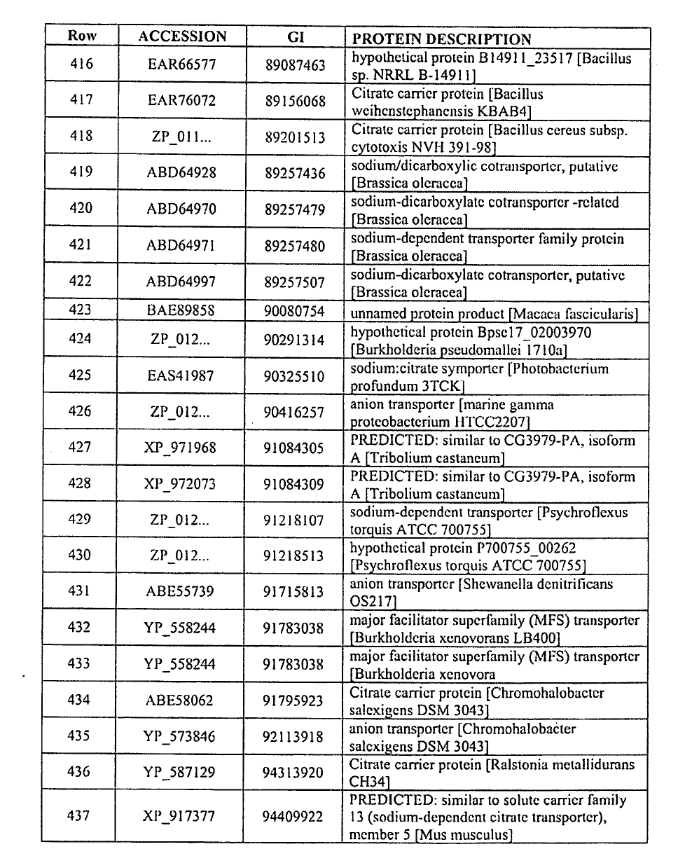

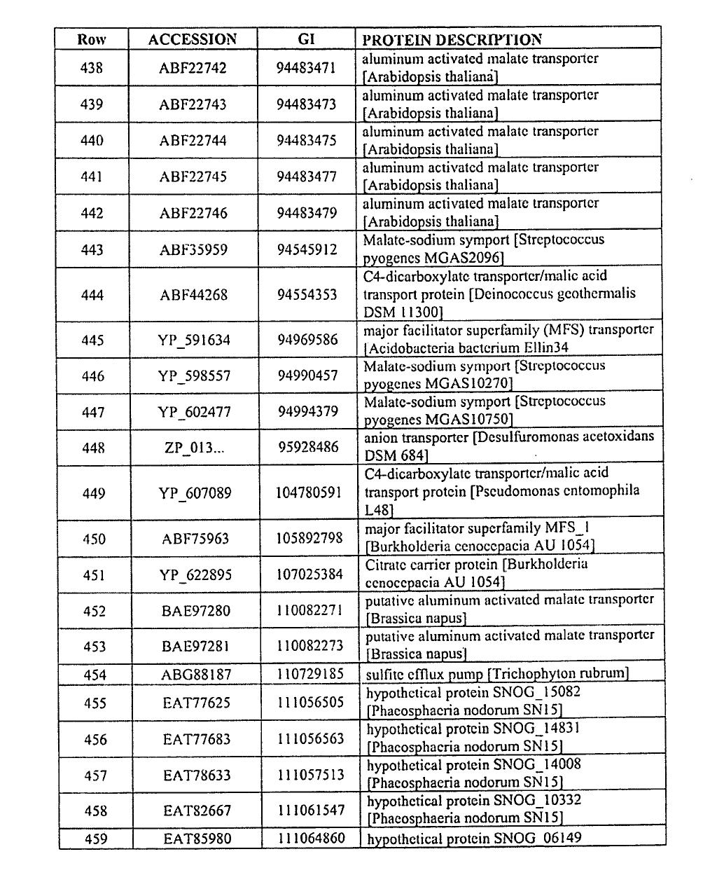

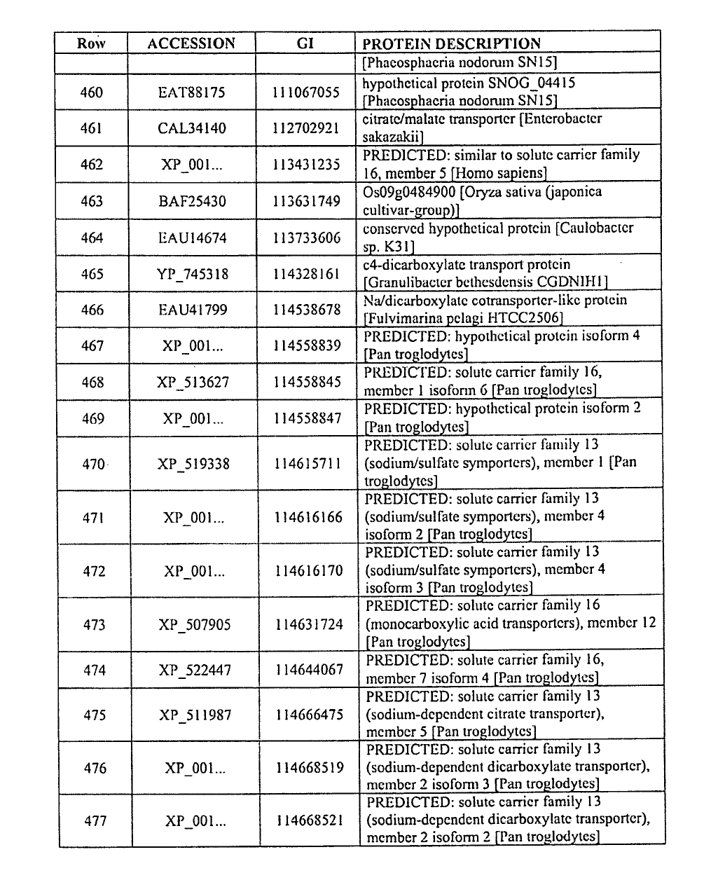

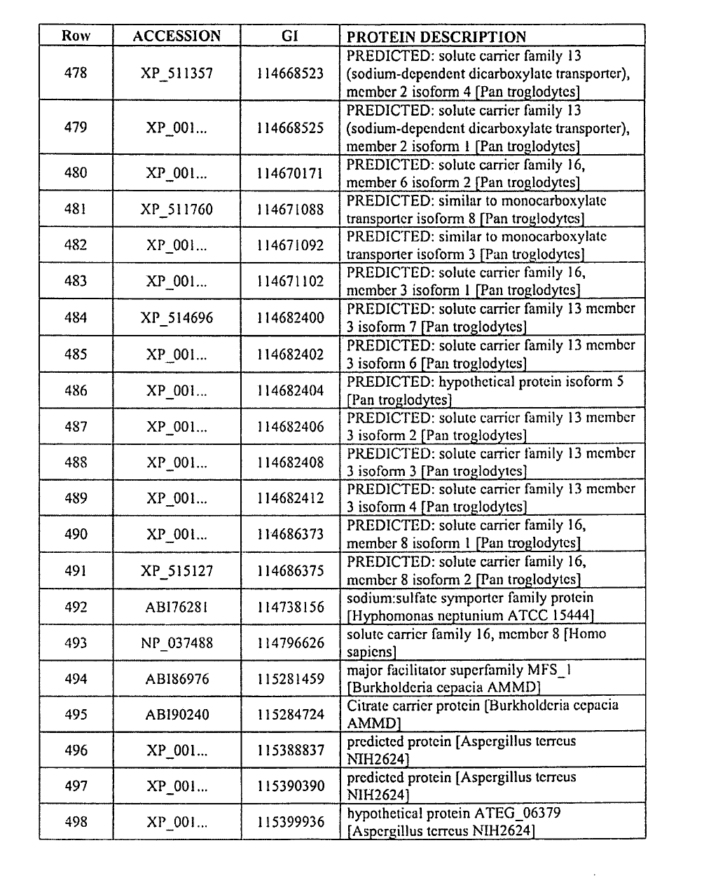

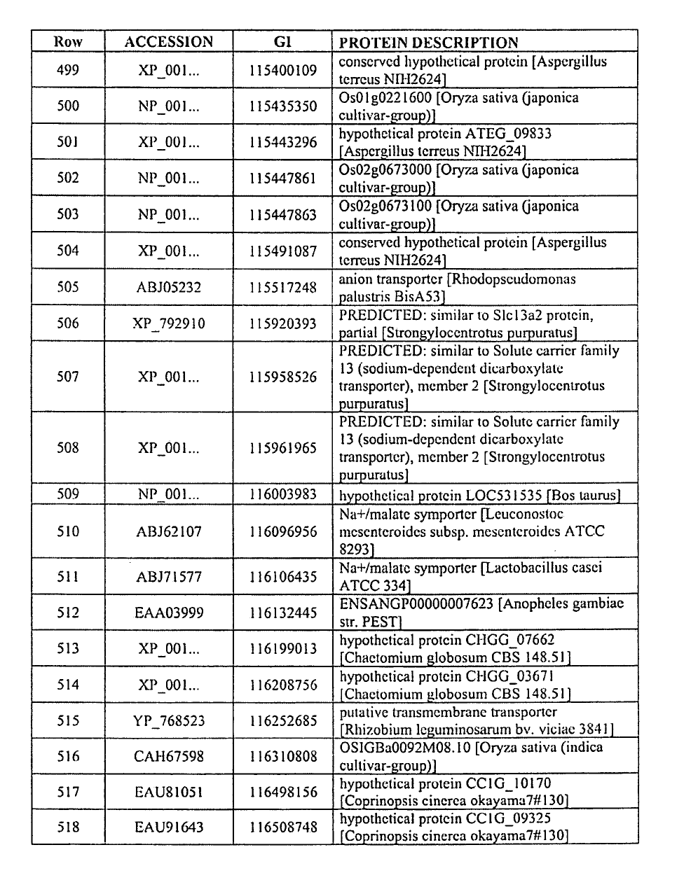

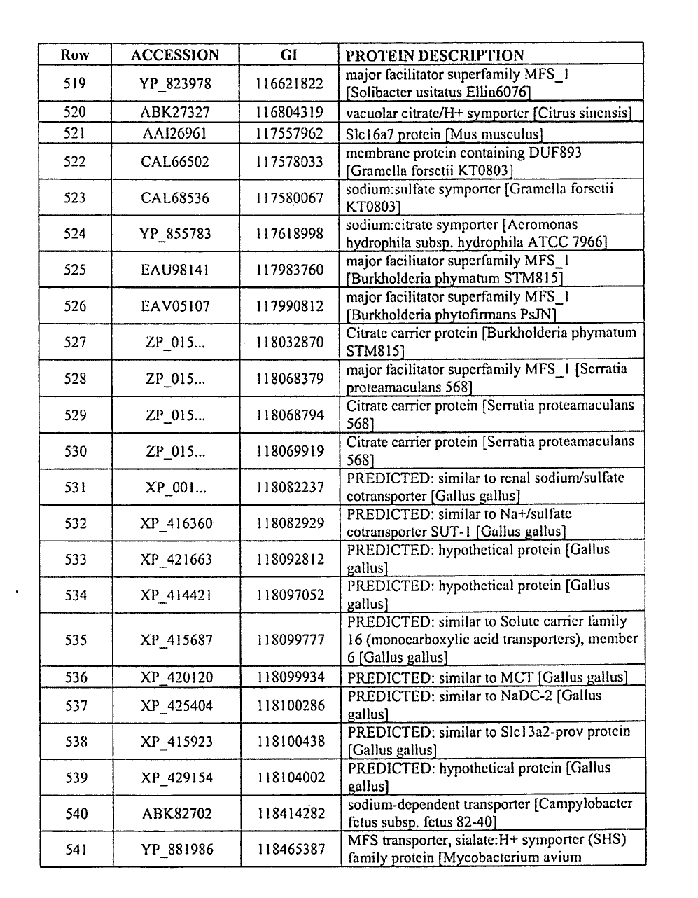

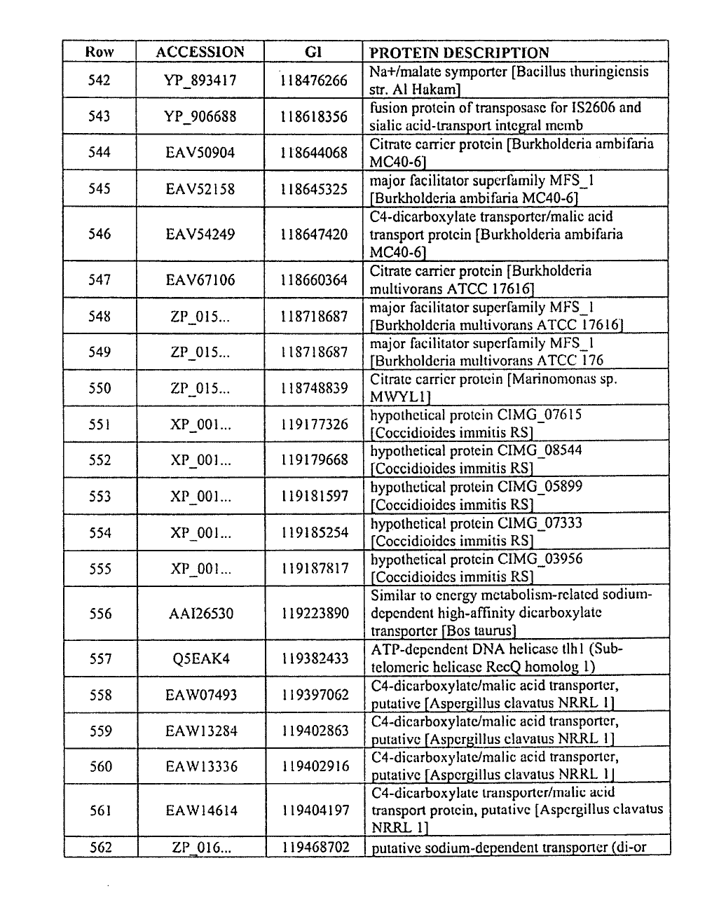

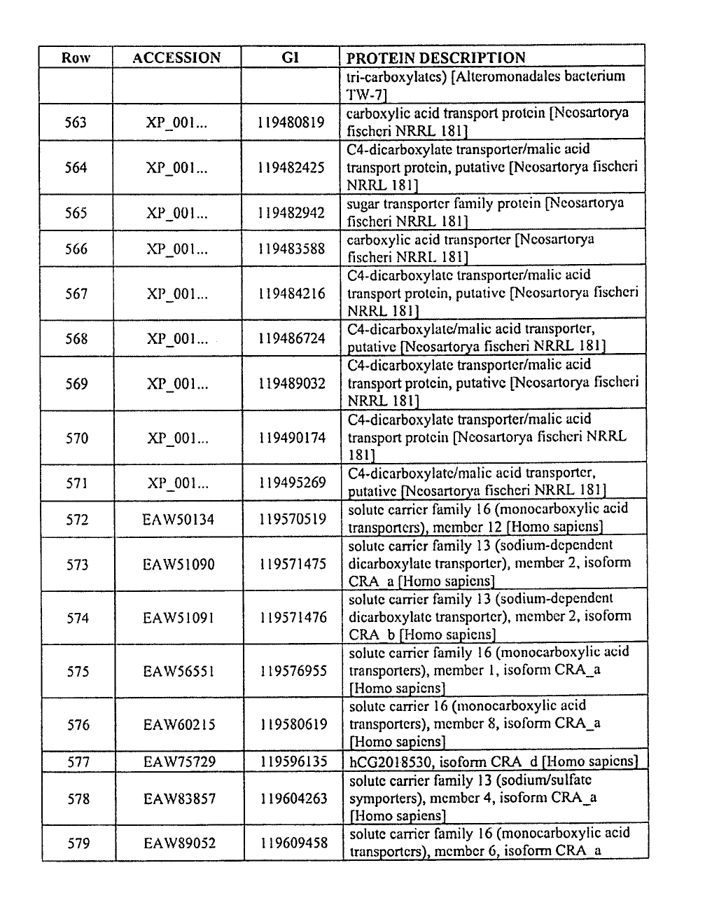

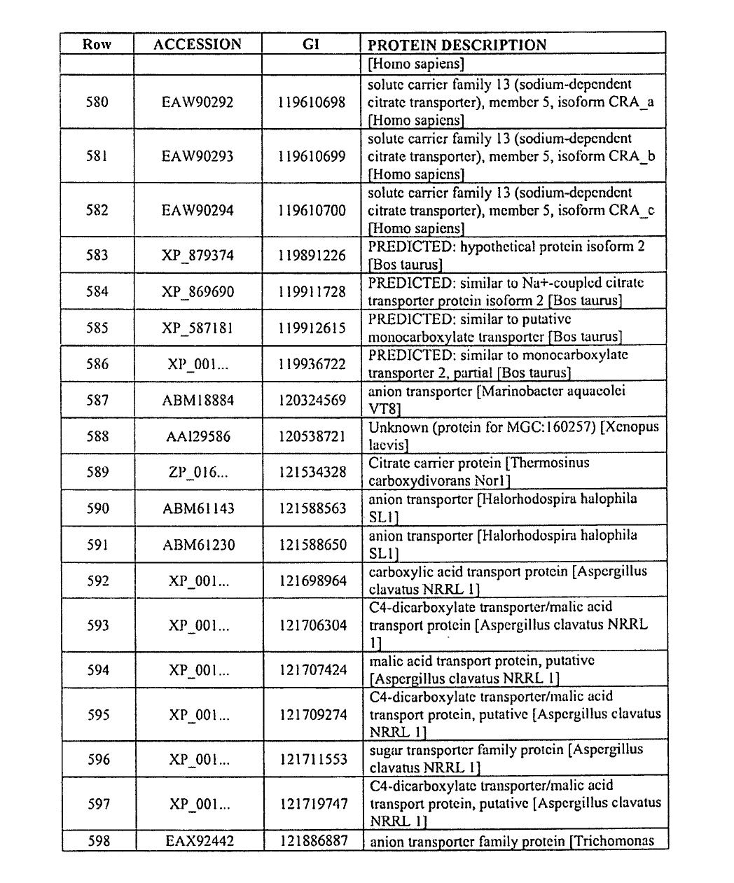

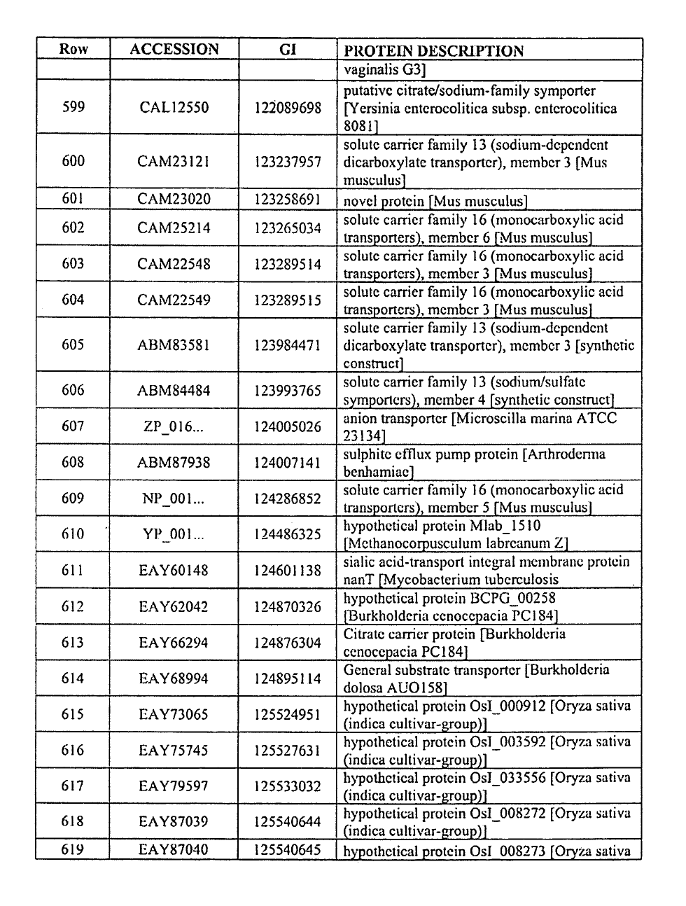

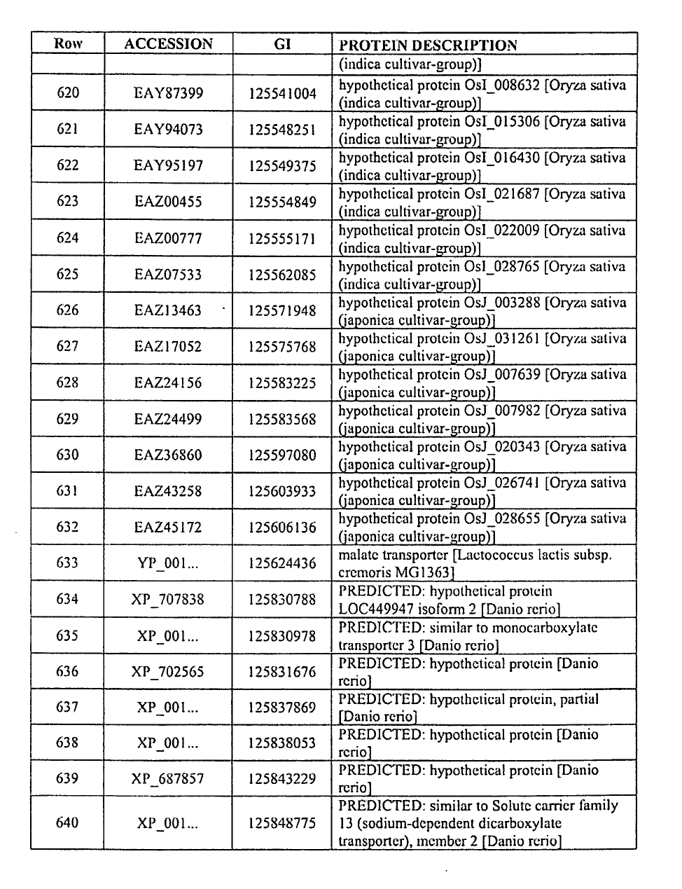

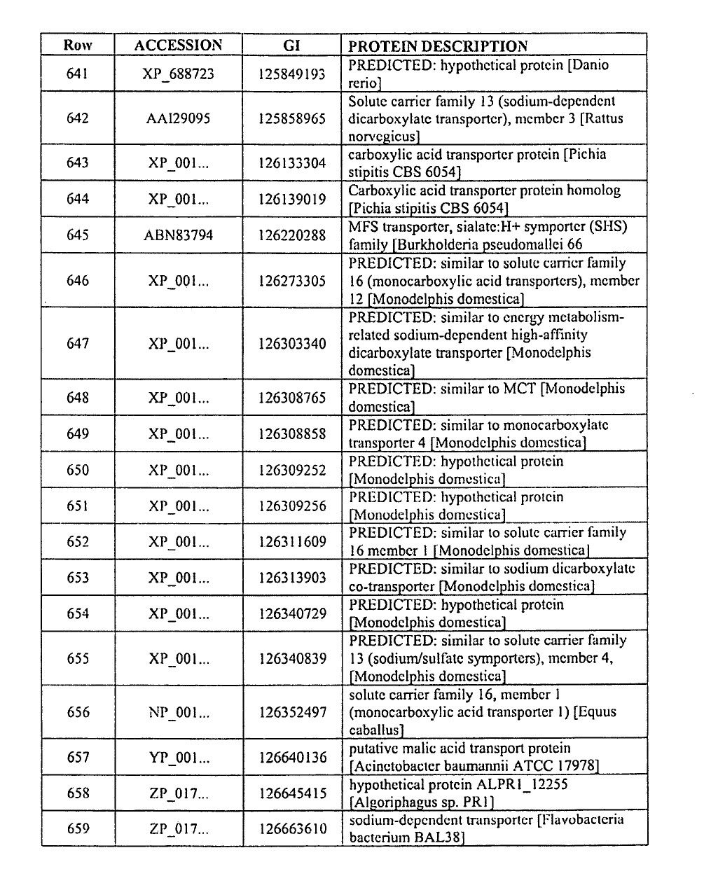

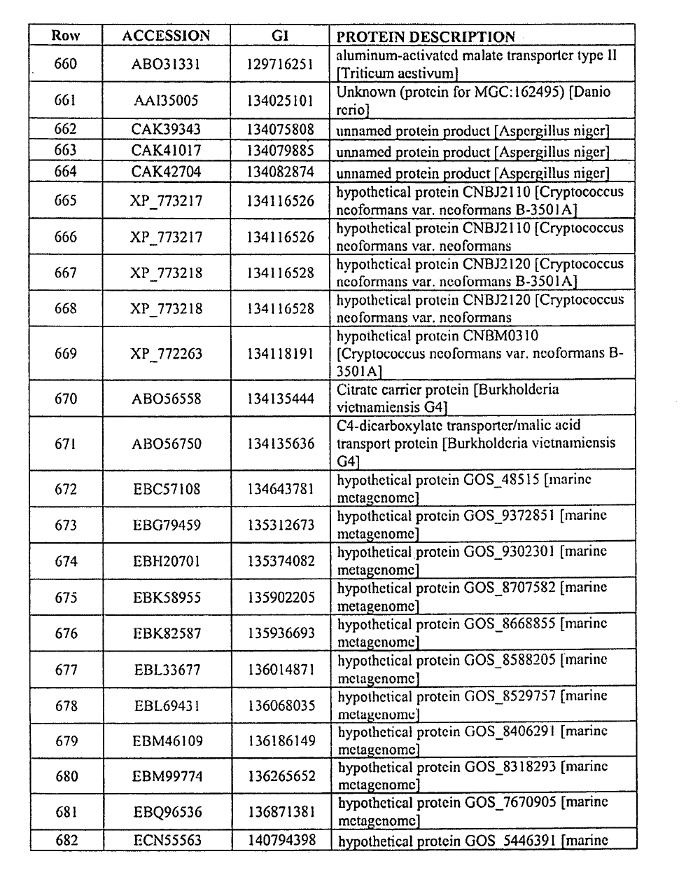

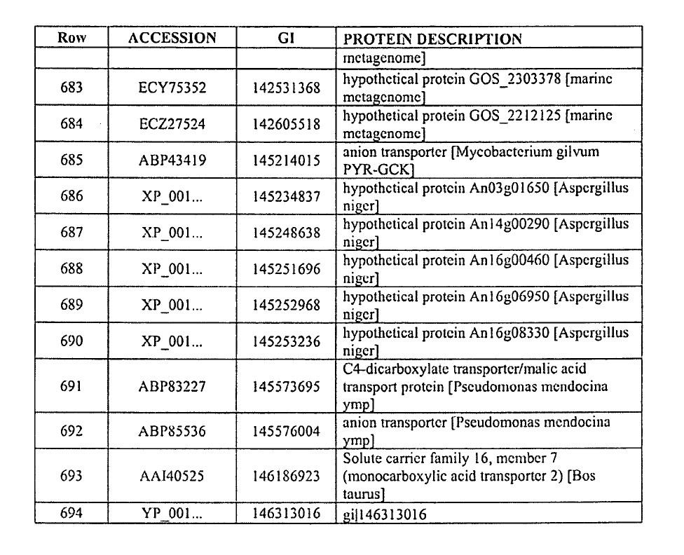

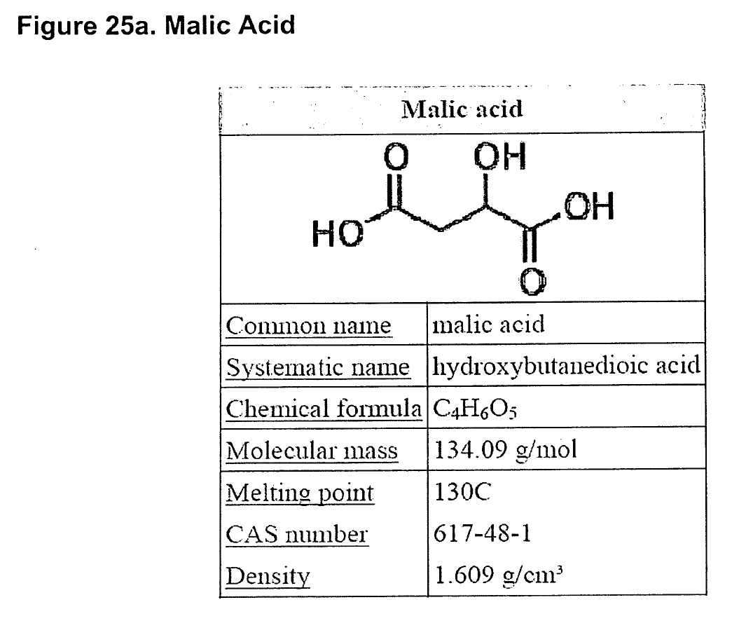

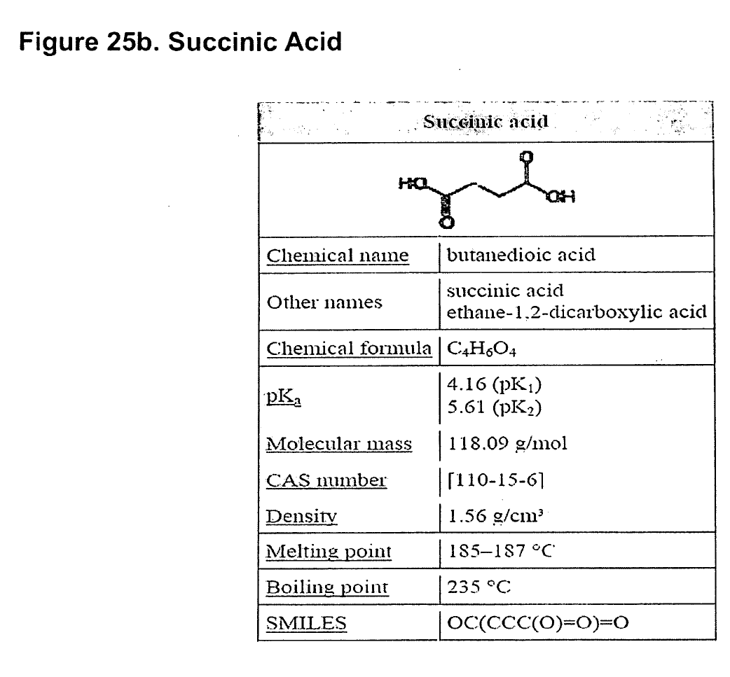

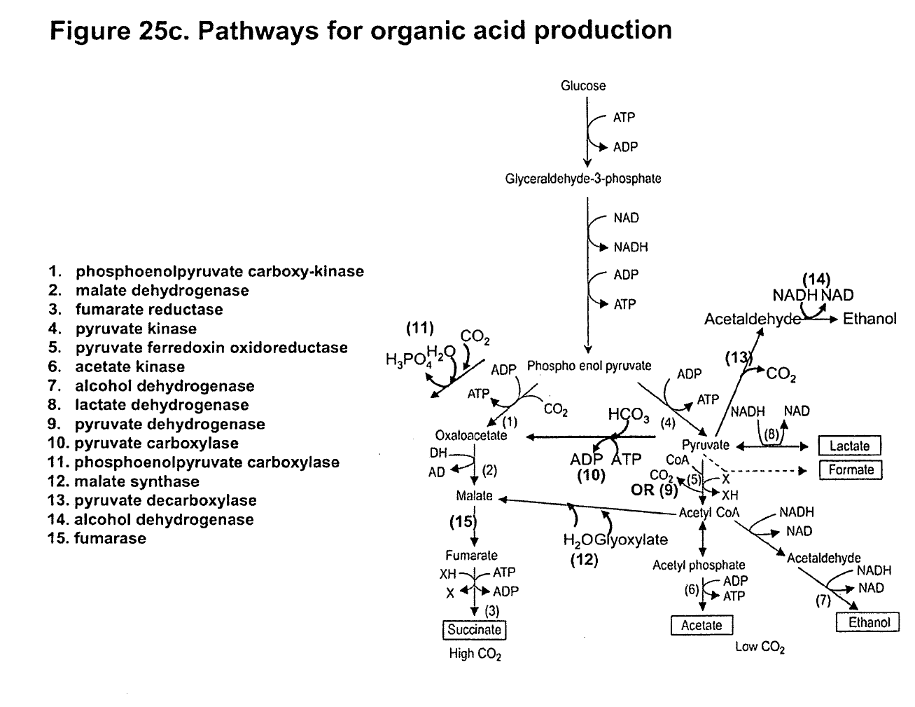

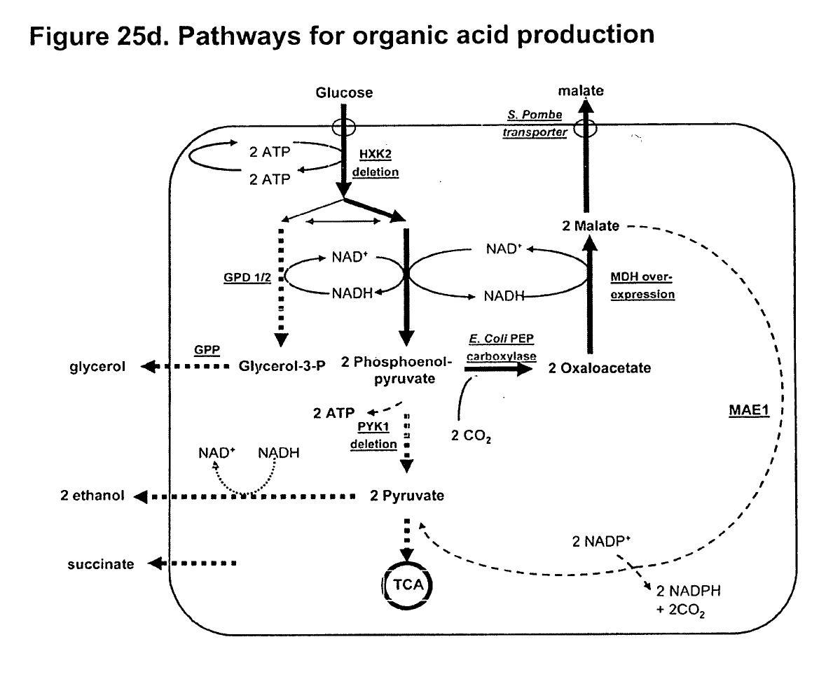

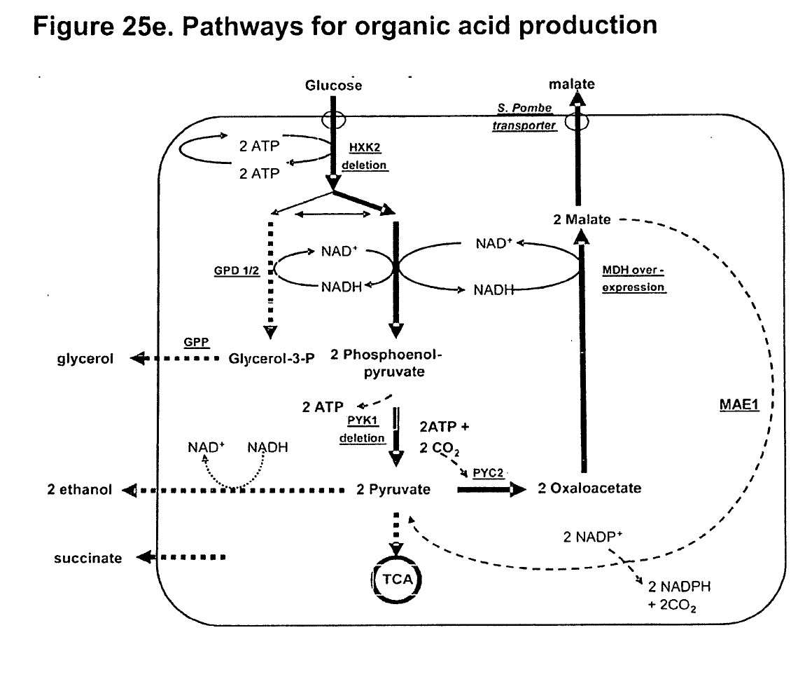

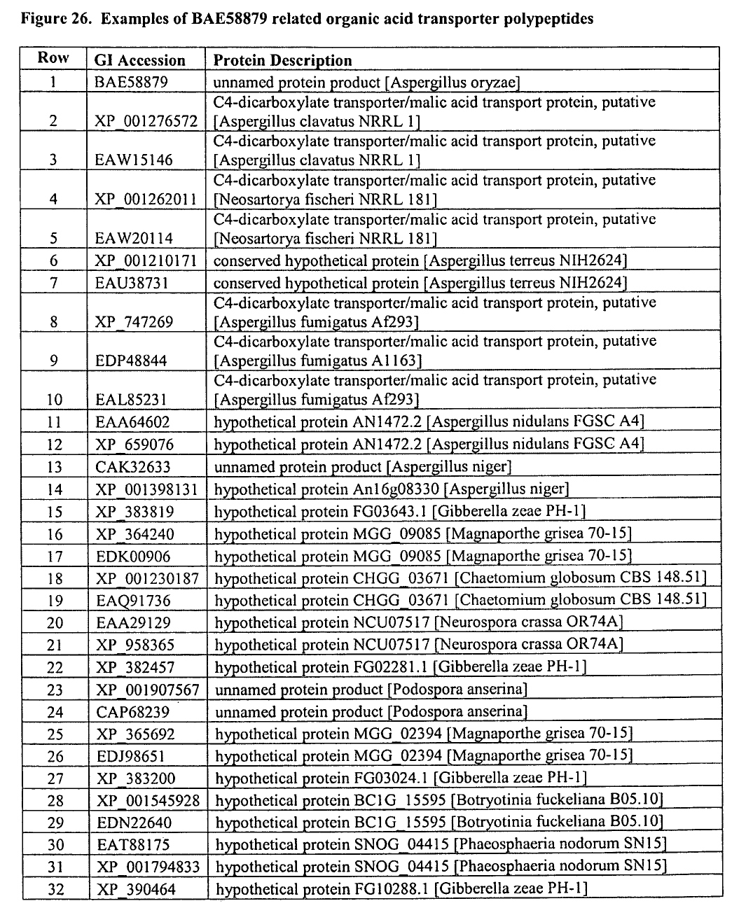

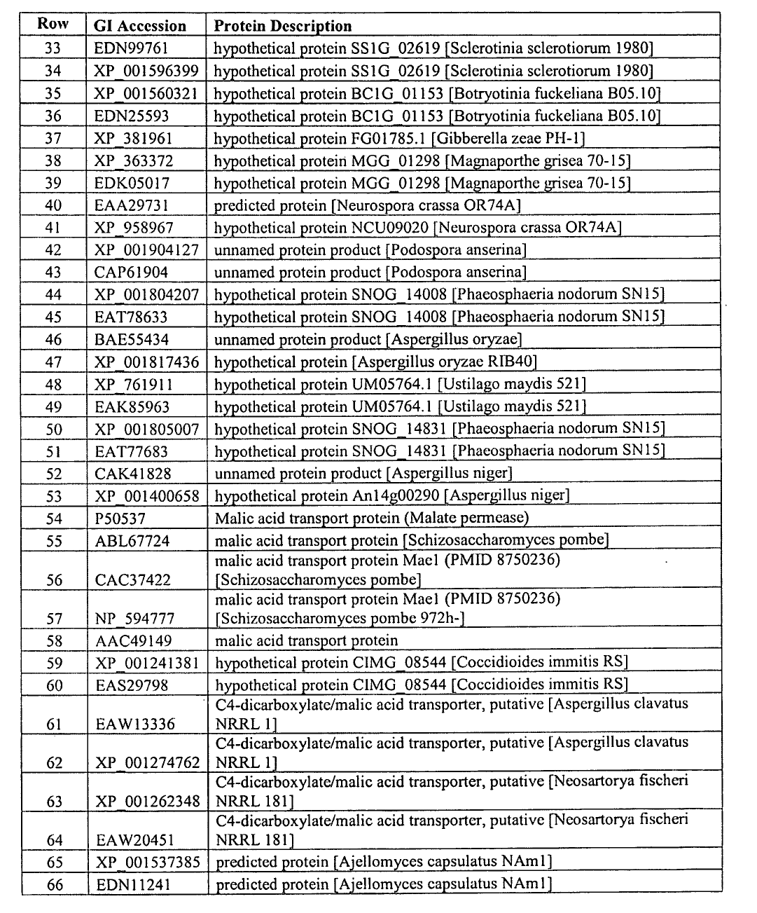

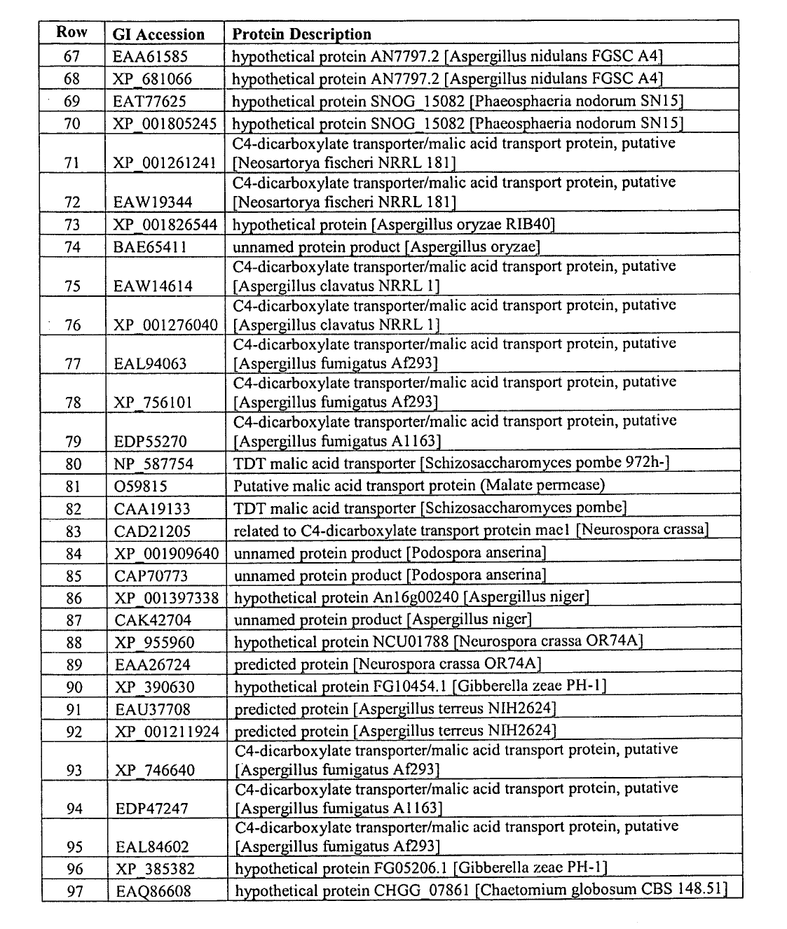

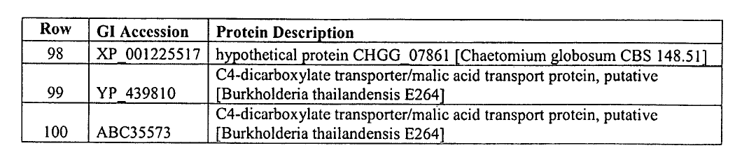

In one embodiment, the MDH polypeptide has at least 75% identity to the amino acid sequence given in SEQ ID NO:2. In one embodiment, the MDH polypeptide has at least 80% identity to the amino acid sequence given in SEQ ID NO:2. In one embodiment, the MDH polypeptide has at least 85% identity to the amino acid sequence given in SEQ ID NO:2. In one embodiment, the MDH polypeptide has at least 90% identity to the amino acid sequence given in SEQ ID NO:2. In one embodiment, the MDH polypeptide has at least 95% identity to the amino acid sequence given in SEQ ID NO:2. In another embodiment, the MDH polypeptide has at least 96% identity to the amino acid sequence given in SEQ ID NO:2. In an additional embodiment, the MDH polypeptide has at least 97% identity to the amino acid sequence given in SEQ ID NO:2. In yet another embodiment, the MDH polypeptide has at least 98% identity to the amino acid sequence'given in SEQ ID NO:2. In still another embodiment, the MDH polypeptide has at least 99% identity to the amino acid sequence given in SEQ ID NO:2. In still yet another embodiment, the MDH polypeptide has the amino acid sequence given in SEQ ID NO:2. In certain embodiments, the malate dehydrogenase (MDH) polypeptide has the amino acid sequence of a malate dehydrogenase in FIG. 18 or has at least 99%, 98%, 97%, 96%, 95%, 94%, 93%, 92%, 91%, 90%, 85%, 80%, 75% identity to a PPC in FIG. 18. In another embodiment, the malate dehydrogenase polypeptide is a polypeptide represented by the. Genbank GI numbers in FIG. 23 or has at least 99%, 98%, 97%, 96%, 95%, 94%, 93%, 92%, 91%, 90%, 85%, 80%, 75% identity to a polypeptide represented by the Genbank GI numbers in FIG. 23.

An organic acid transport (MAE) polypeptide for use in accordance with the present disclosure can be any protein capable of transporting an organic acid (e.g., malate or succinate) from the cytosol of a yeast across the cell membrane and into extracellular space and/or from the extracellular space across the cell membrane into the cystosol. A protein need not be identified in the literature as an organic acid transport at the time of filing of the present application to be within the definition of an MAE.

A MAE polypeptide from any source organism may be used and the MAE polypeptide may be wild type or modified from wild type. The MAE polypeptide can be Schizosaccharomyces pombe SpMAE1. In one embodiment, the MAE polypeptide has at least 75% identity to the amino acid sequence given in SEQ ID NO:3. In one embodiment, the MAE polypeptide has at least 80% identity to the amino acid sequence given in SEQ ID NO:3. In one embodiment, the MAE polypeptide has at least 85% identity to the amino acid sequence given in SEQ. ID NO:3. In one embodiment, the MAE polypeptide has at least 90% identity to the amino acid sequence given in SEQ ID NO:3. In one embodiment, the MAE polypeptide has at least 95% identity to the amino acid sequence given in SEQ ID NO:3. In another embodiment, the MAE polypeptide has at least 96% identity to the amino acid sequence given in SEQ ID NO:3. In an additional embodiment, the MAE polypeptide has at least 97% identity to the amino acid sequence given in SEQ ID NO:3. In yet another embodiment, the MAE polypeptide has at least 98% identity to the amino acid sequence given in SEQ ID NO:3. Instill another embodiment, the MAE polypeptide has at least 99% identity to the amino acid sequence given in SEQ ID NO:3. In still yet another embodiment, the MAE polypeptide has the amino acid sequence given in SEQ ID NO:3. In certain embodiments, the organic acid transport (MAE) polypeptide has the amino acid sequence of an organic acid transport polypeptide in FIG. 18 or has at least 99%, 98%, 97%, 96%, 95%, 94%, 93%, 02%, 91%, 90%, 85%, 80%, 75% identity to an organic acid transport polypeptide in FIG. 18. In another embodiment, the organic acid transport polypeptide is a polypeptide represented by the Genbank GI numbers in FIG. 24 or has at least 99%, 98%, 97%, 96%, 95%, 94%, 93%, 92%, 91%, 90%, 85%, 80%, 75% identity to a polypeptide represented by the Genbank GI numbers in FIG. 24. In another embodiment, the transporter polypeptide comprises or consists of an amino acid polypeptide that is at least 99%, 98%, 97%, 96%, 95%, 94%, 93%, 92%, 91%, 90%, 85%, 80%, 75% identical to A. oryzae organic acid transporter. to a polypeptide represented by the Genbank GI numbers in FIG. 24. Add the preferred DCAT as well.

In some embodiments, the present disclosure provides modified yeast cells that have a first modification that reduces PDC polypeptide activity and at least one additional modification selected from the group consisting of a second modification that increases pyruvate carboxylase (PYC) polypeptide activity, a third modification that increases phosphoenolpyruvate carboxylase polypeptide activity (“PPC activity”), a fourth modification that increases malate dehydrogenase (MDH) polypeptide activity, and/or a fifth modification that increases organic acid transport (MAE) polypeptide activity. In some embodiments, the modified yeast has at least two of the second, third, fourth, and fifth modifications. In some embodiments, the modified yeast has at least three of the second, third, fourth and fifth modifications. In some embodiments, the modified yeast has all of the second, third, fourth, and fifth modifications.

In some embodiments of this aspect of the present disclosure, at least one of the second, third, fourth, and fifth modifications comprises a genetic modification; in at least some embodiments, such a genetic modification comprises introducing into a yeast cell a gene encoding the relevant polypeptide. In some embodiments, the introduced gene is from a source (i.e., has an amino acid sequence identical to that found in a source organisms) selected from the group consisting of Saccharomyces cerevisiae, Yarrowia lipolytica, Aspergillus oryzae, Aspergillus niger, Nocardia sp. JS614, Methanothermobacter thermautotrophicus str. Delta H, Actinobacillus succinogenes, Actinobacillus pleuropneumoniae, Escherichia coli, Erwinia carotovora, Erwinia chrysanthemi, (Thermo)synechococcus vulcanus, Streptococcus bovis, Corynebacterium glutamicum, Arabidopsis thaliana, Brassica napus, Triticum secale, Rattus norvegicus, Mus musculus or Homo sapiens. In some embodiments, each such gene is from the same source; in some embodiments, different genes are from different sources.

A nucleic acid to be transformed into a host cell according to the present disclosure may be prepared by any available means. For example, it may be extracted from an organism's nucleic acids or synthesized by chemical means. Such a nucleic acid may by inserted into a vector, or may be introduced directly into yeast cells without such insertion. Insertion into a vector can involve the use of restriction endonucleases to “open up” the vector at a desired point where operable linkage to the promoter is possible, followed by ligation of the coding region into the desired point. In some embodiments, such insertion may also involve operative association with a promoter (and/or at least one other regulatory element) that is active in yeast cells. Any promoter active in the target host (homologous or heterologous; constitutive, inducible or repressible) can be used in accordance with the present disclosure.

FIGS. 18-24 and 26 are tables referenced throughout the description. Each reference and information designated by each of the Genbank Accession and GI numbers are hereby incorporated by reference in their entirety. The entries in the tables are organized for convenient reference and the order is not intended to reflect preferences for certain nucleotide or amino acid sequences.

A nucleic acid of interest may be introduced into a host cell together with at least one detectable marker (e.g., a screenable or selectable marker). In some embodiments, a single nucleic acid molecule to be introduced may include both a sequence of interest (e.g., a gene encoding a polypeptide of interest as described herein) and a detectable marker. In general, a detectable marker allows transformed cells to be distinguished from untransformed cells. For example, a selectable marker may allow transformed cells to survive in a medium comprising an antibiotic fatal to untransformed yeast, or may allow transformed cells to metabolize a component of the medium into a product not produced by untransformed cells, among other phenotypes.

If desired, a nucleic acid to be introduced into and expressed within a host cell can be prepared for use in the target organism prior to such introduction. This can involve altering the codons used in the coding region to more fully match the codon use of the target organism; changing sequences in the coding region that could impair the transcription or translation of the coding region or the stability of an mRNA transcript of the coding region; or adding or removing portions encoding signaling peptides (regions of the protein encoded by the coding region that direct the protein to specific locations (e.g. an organelle, the membrane of the cell or an organelle, or extracellular secretion)), among other possible preparations known in the art.

A promoter, as is known, is a DNA sequence that can direct the transcription of a nearby coding region. As already described, a promoter utilized in accordance with the present disclosure can be constitutive, inducible or repressible. Constitutive promoters continually direct the transcription of a nearby coding region. Inducible promoters can be induced by the addition to the medium of an appropriate inducer molecule, which will be determined by the identity of the promoter. Repressible promoters can be repressed by the addition to the medium of an appropriate repressor molecule, which will be determined by the identity of the promoter. In one embodiment, the promoter is constitutive. For example, in a further embodiment, the constitutive promoter is the S. cerevisiae triosephosphateisomerase (TPI) promoter. For another example, in other embodiments, the promoter can be the S. cerevisiae glyceraldehyde-3-phosphate dehydrogenase (isozyme 3) TDH3 promoter, the S. cerevisiae TEF1 promoter or the S. cerevisiae ADH1 promoter.

A terminator region can be used, if desired. An exemplary terminator region is S. cerevisiae CYC1.

Techniques for yeast transformation are well established, and include electroporation, microprojectile bombardment, and the LiAc/ssDNA/PEG method, among others. Yeast cells, which are transformed, can then be detected by the use of a screenable or selectable marker on the vector. It should be noted that the phrase “transformed yeast” has essentially the same meaning as “recombinant yeast,” as defined above. The transformed yeast can be one that received the vector in a transformation technique, or can be a progeny of such a yeast. Much is known about the different gene regulatory requirements, protein targeting sequence requirements, and cultivation requirements, of different host cells to be utilized in accordance with the present disclosure (see, for example, with respect to Yarrowia, Barth et al. FEMS Microbiol Rev. 19:219, 1997; Madzak et al. J Biotechnol. 109:63, 2004; see, for example, with respect to Saccharomyces, Guthrie and Fink Methods in Enzymology 194:1-933, 1991).

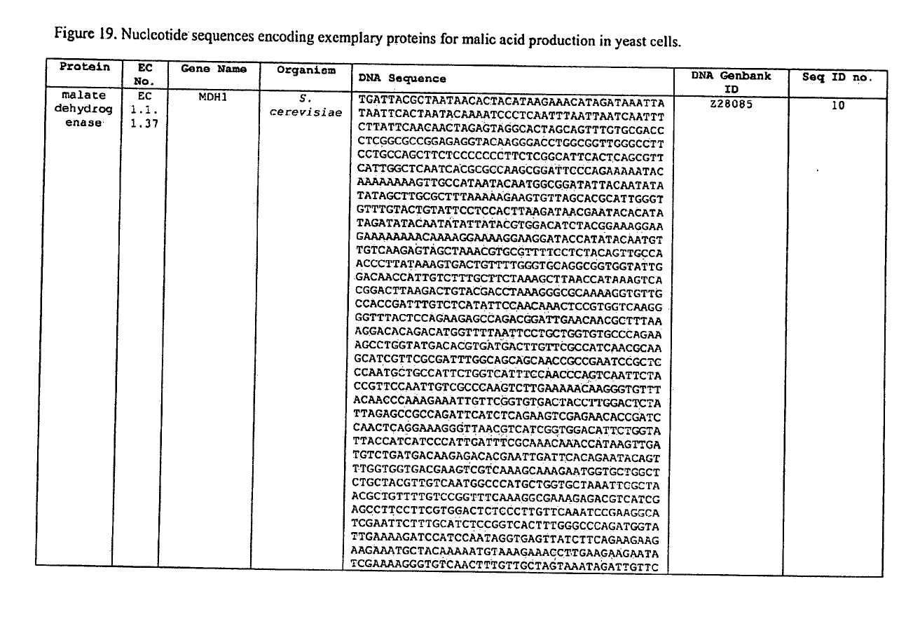

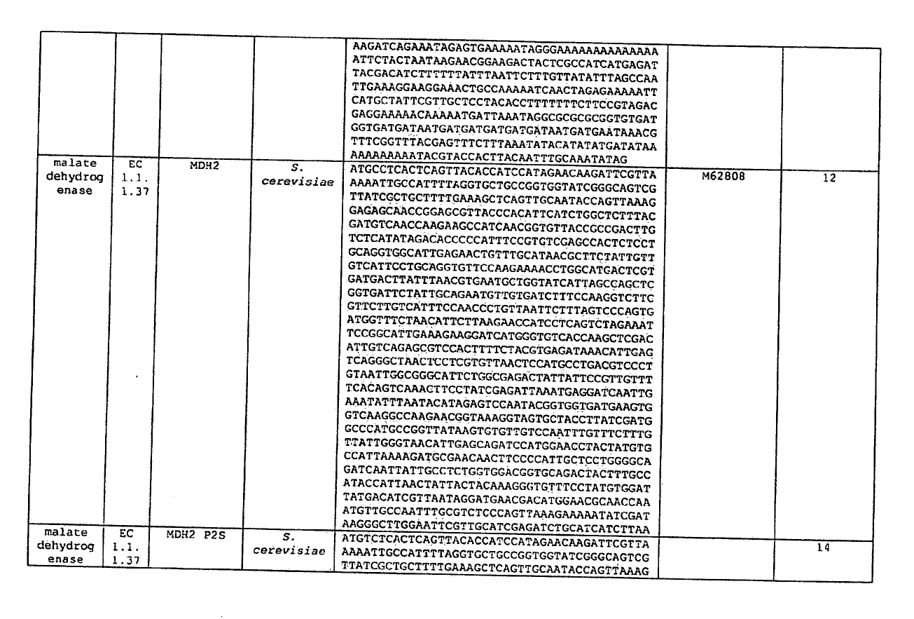

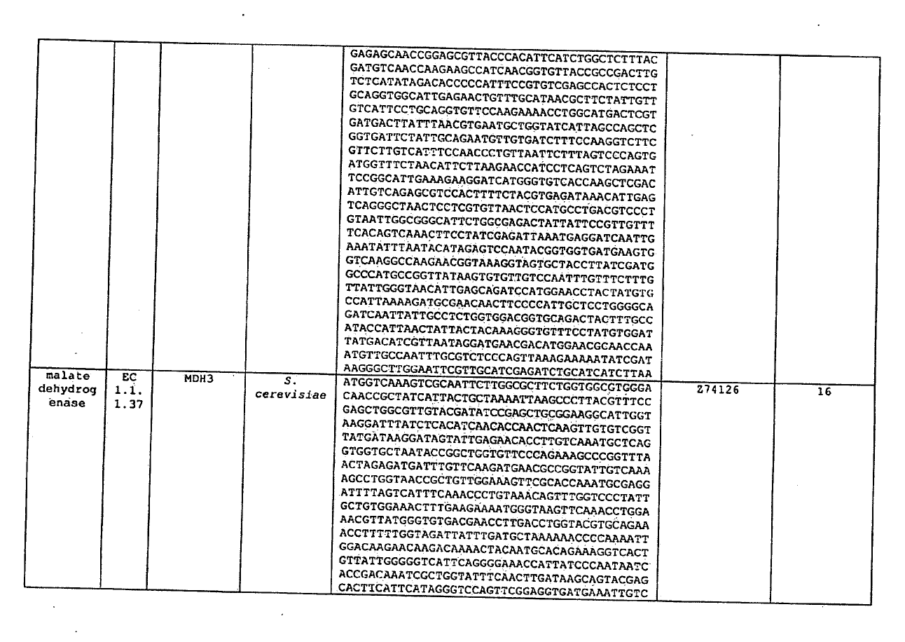

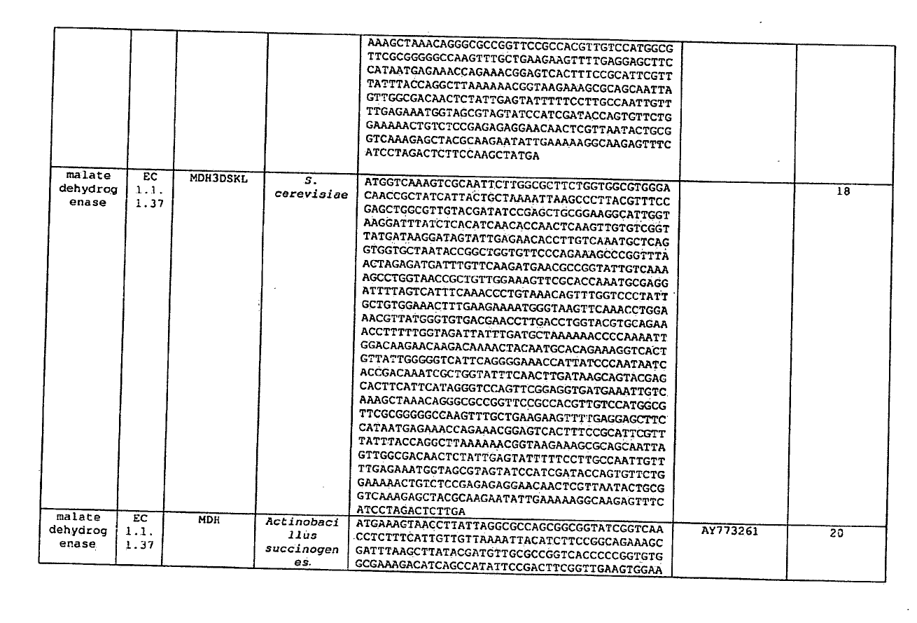

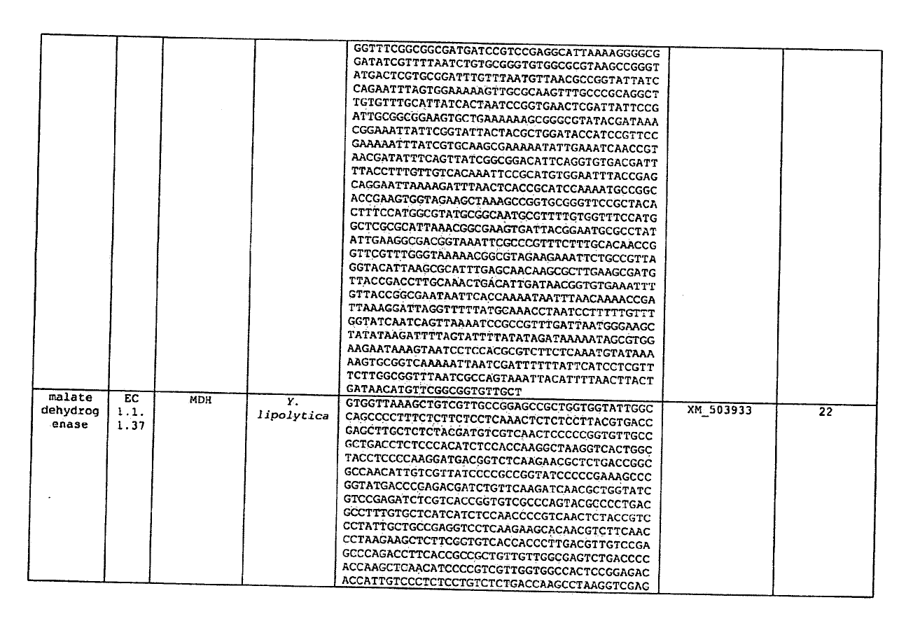

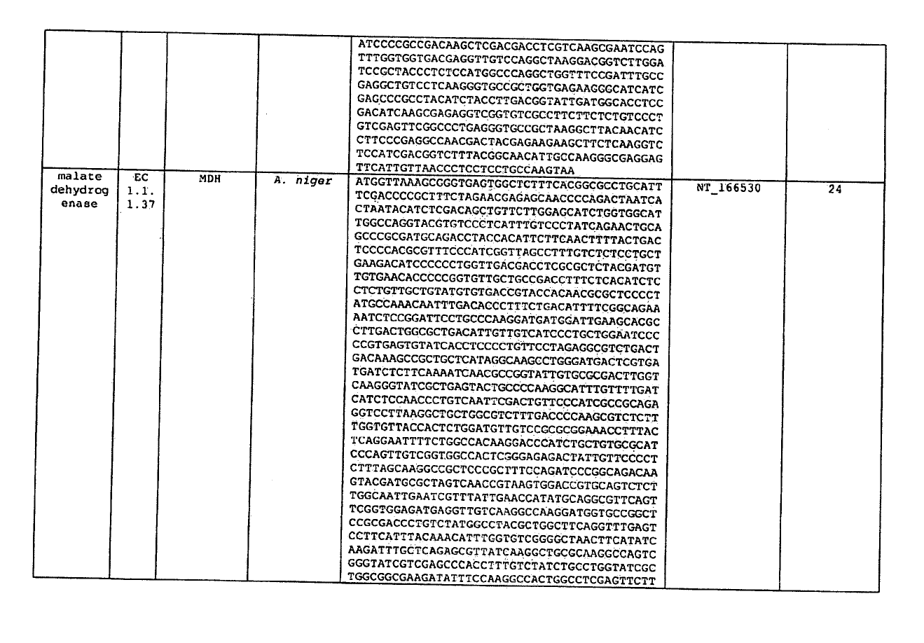

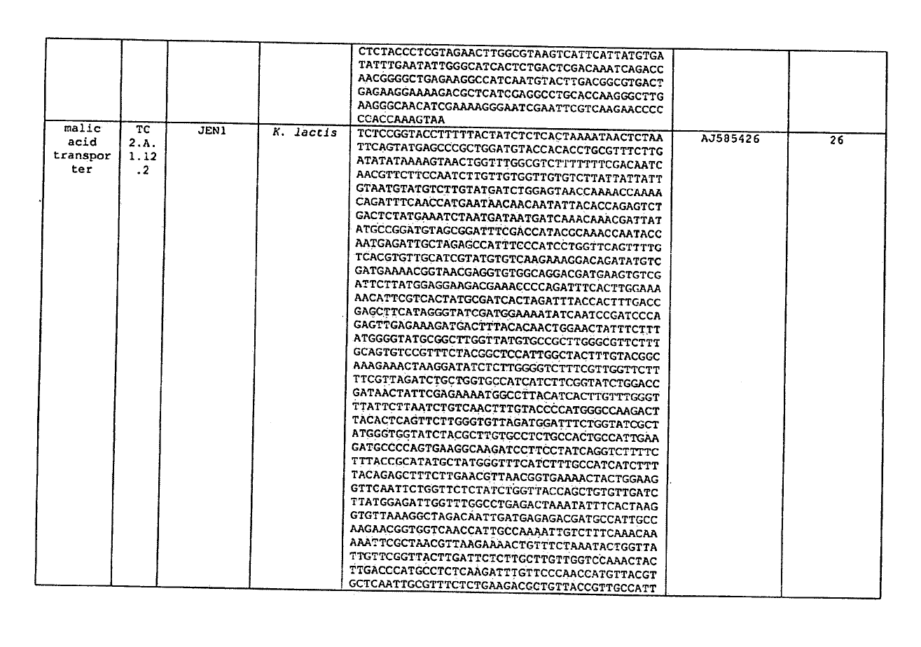

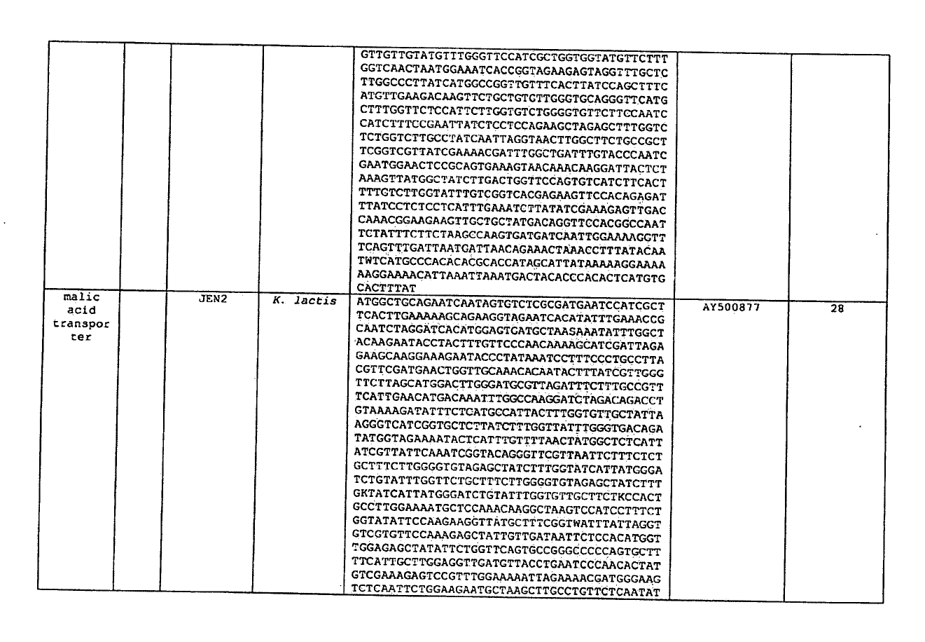

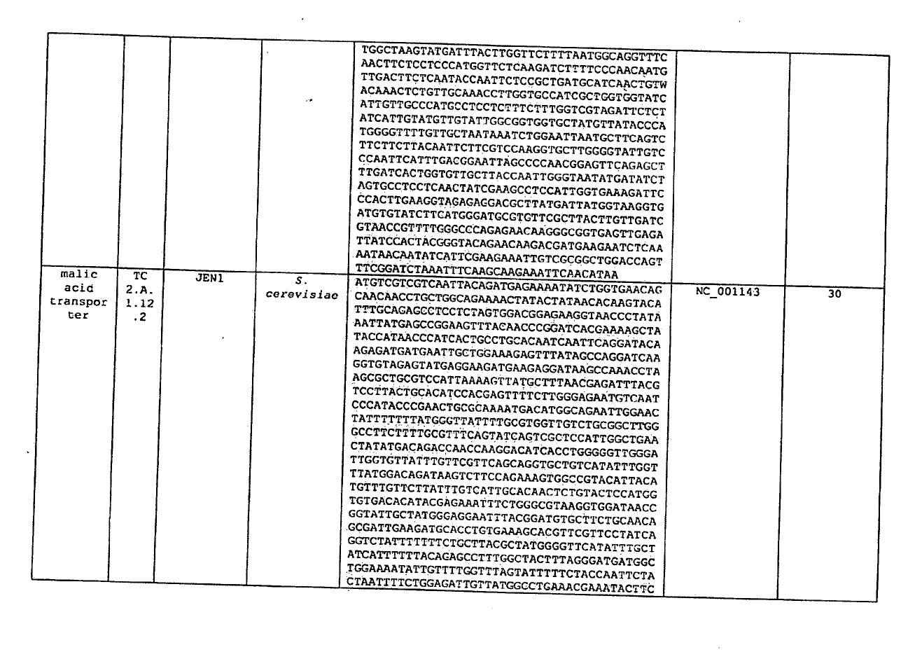

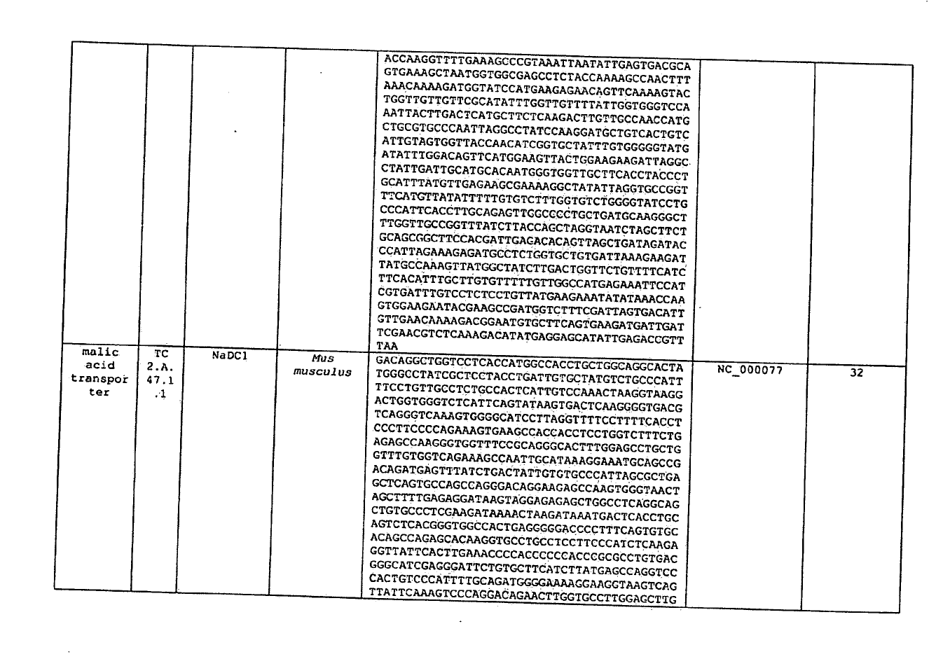

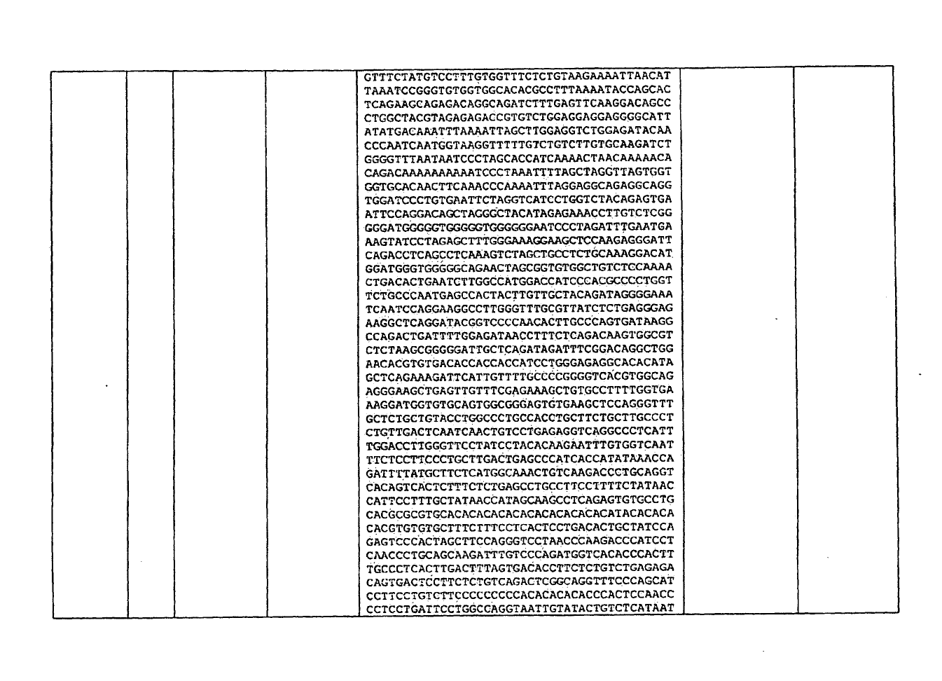

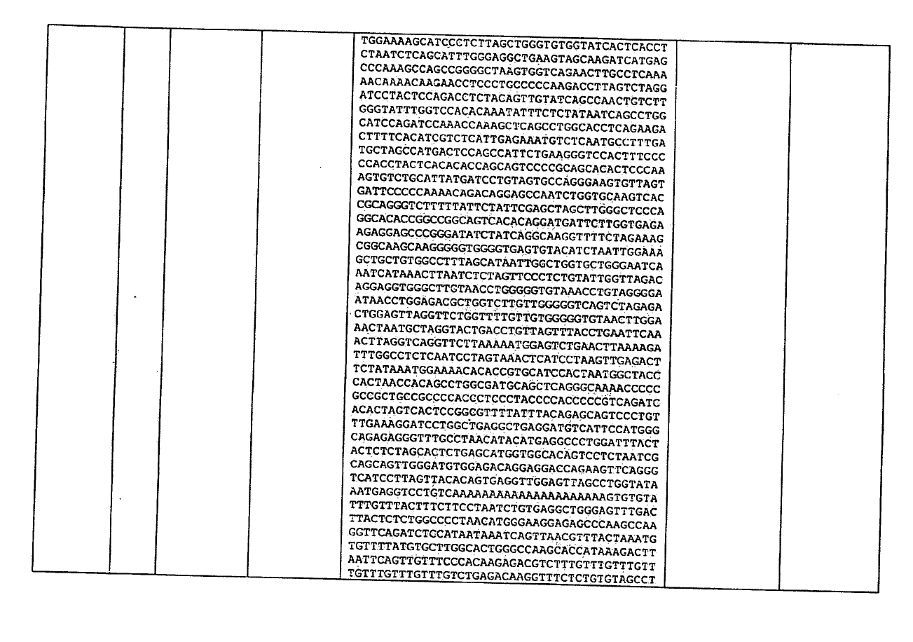

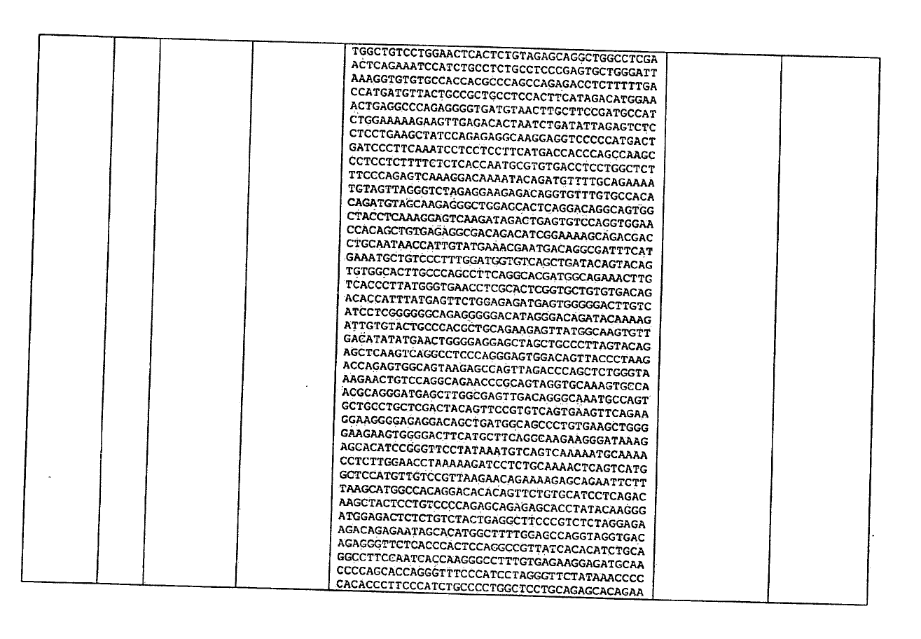

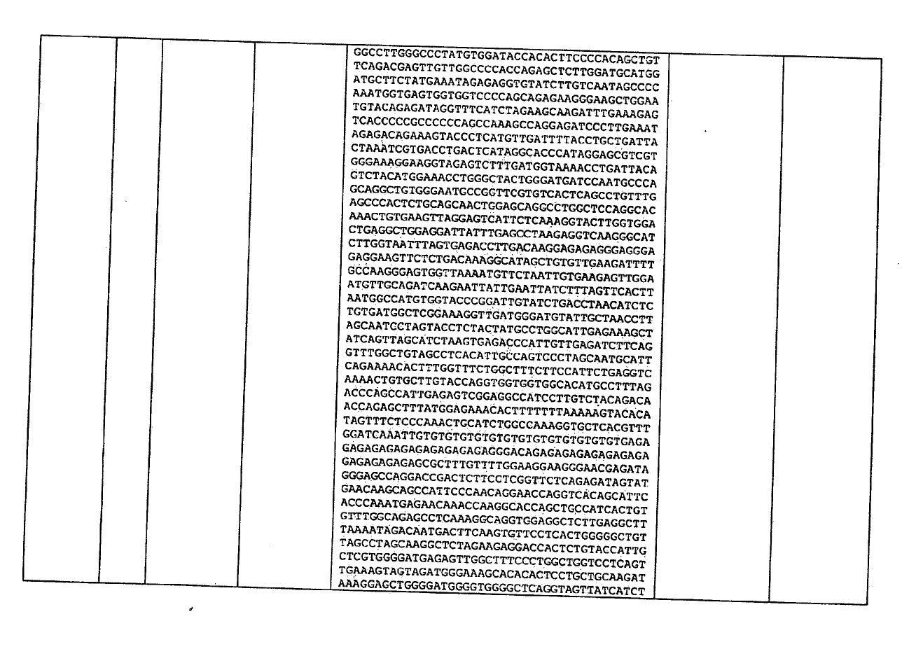

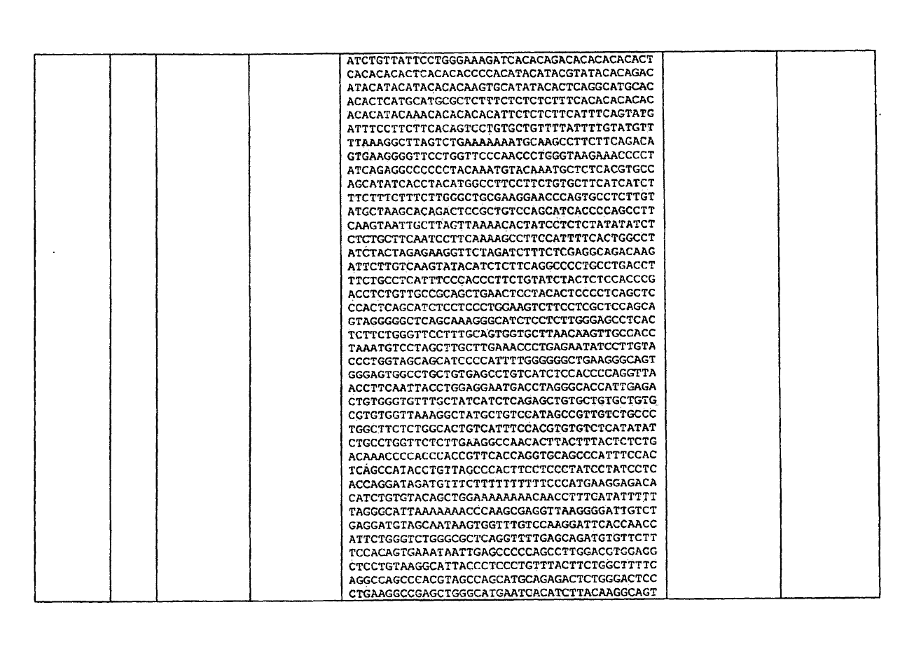

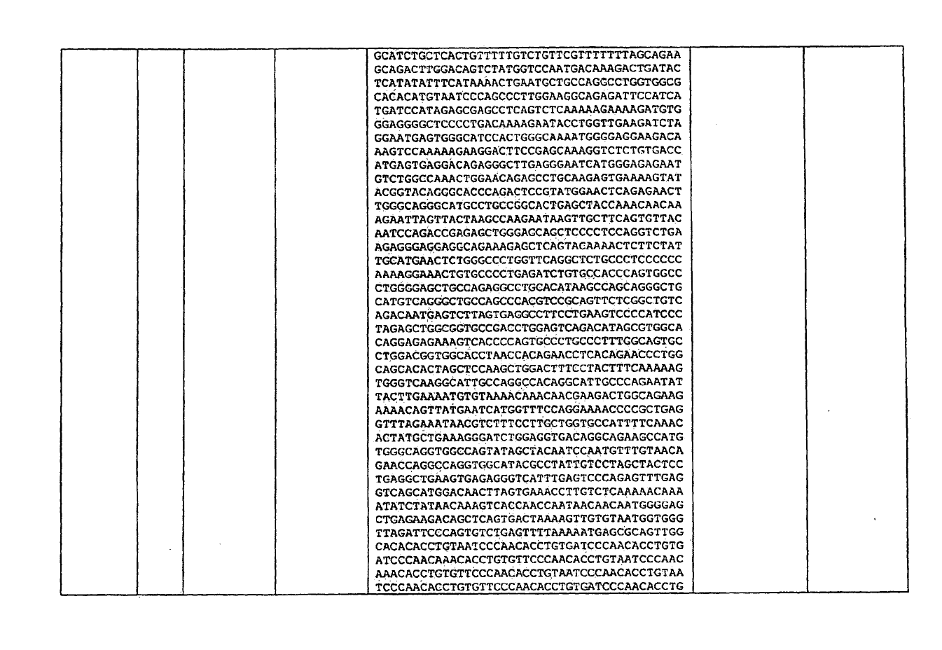

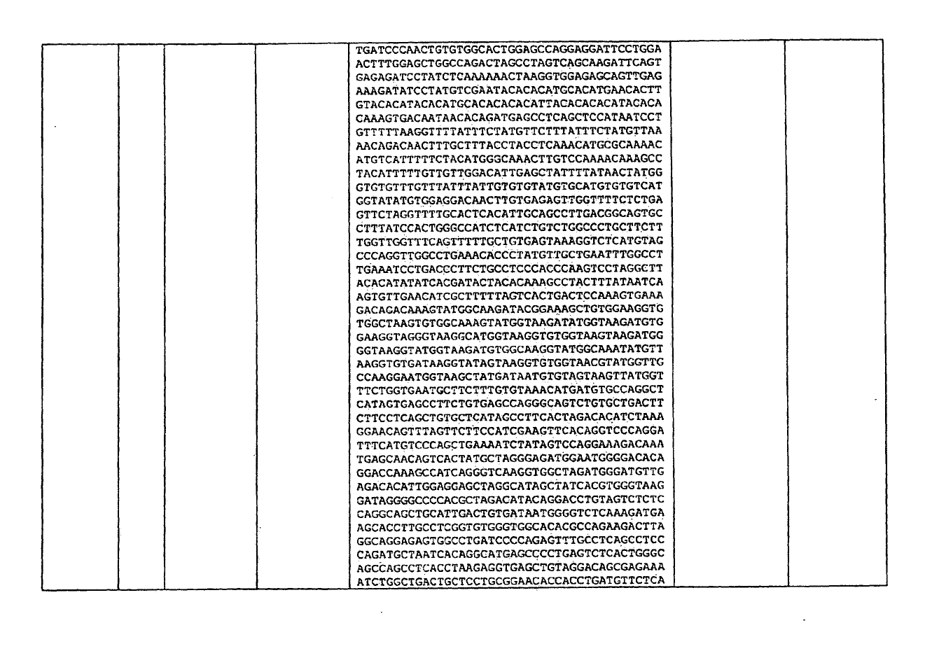

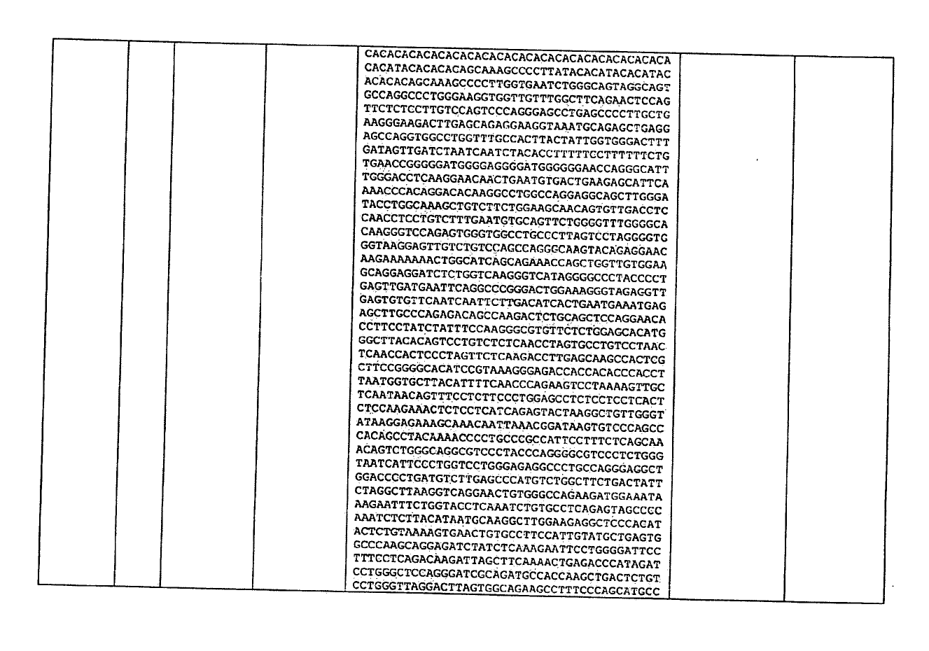

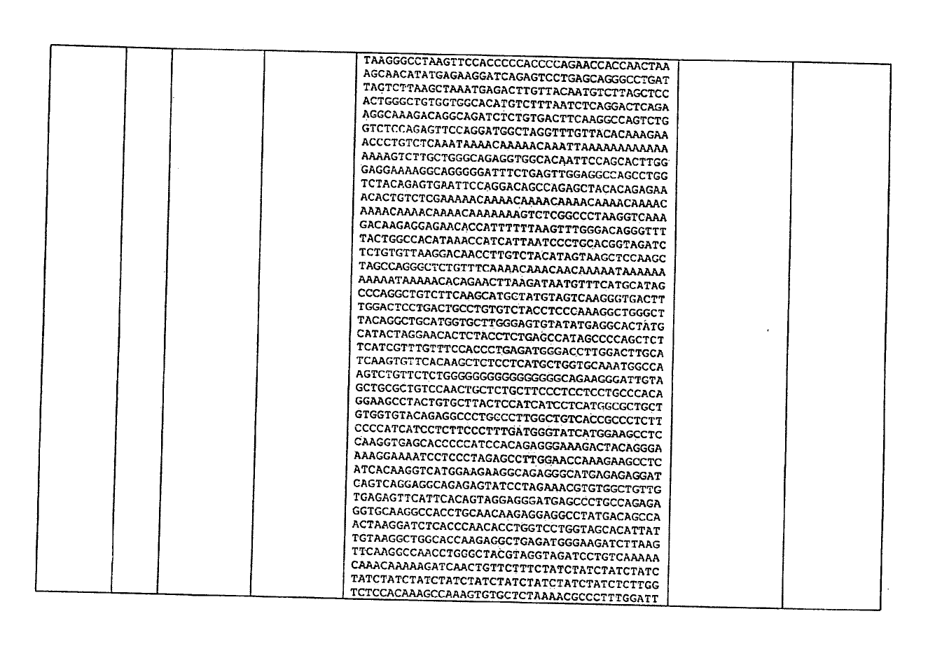

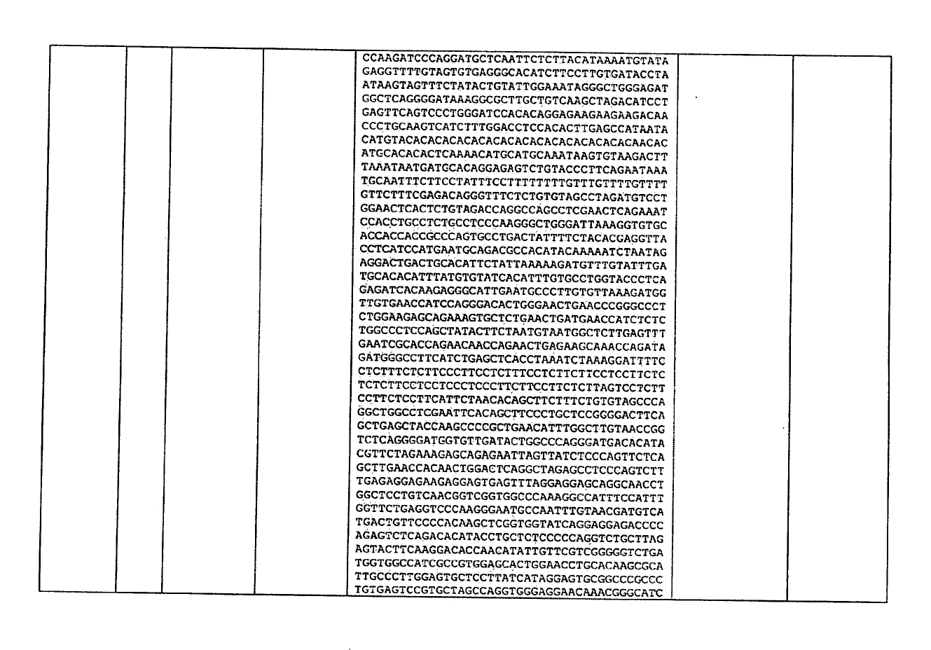

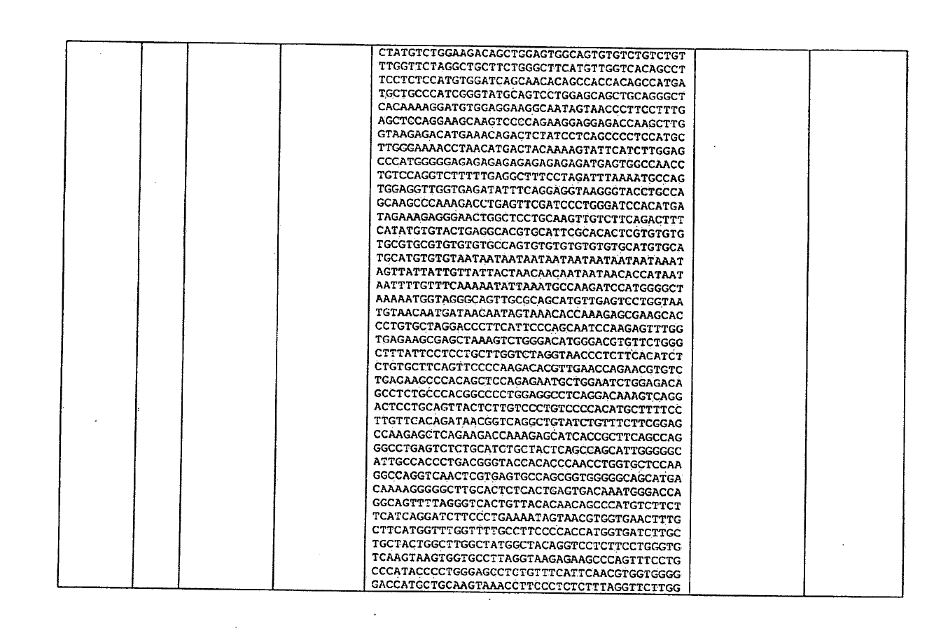

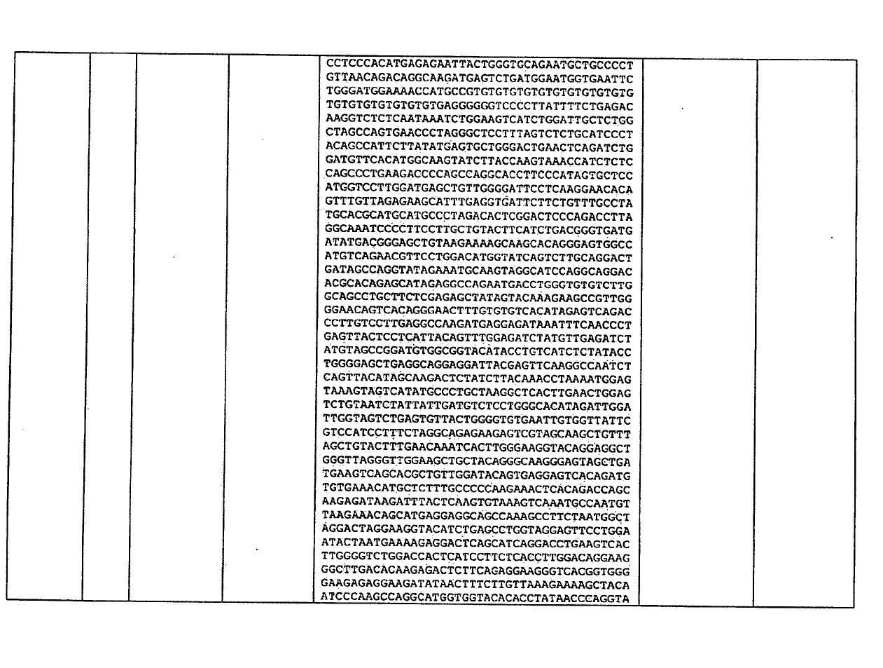

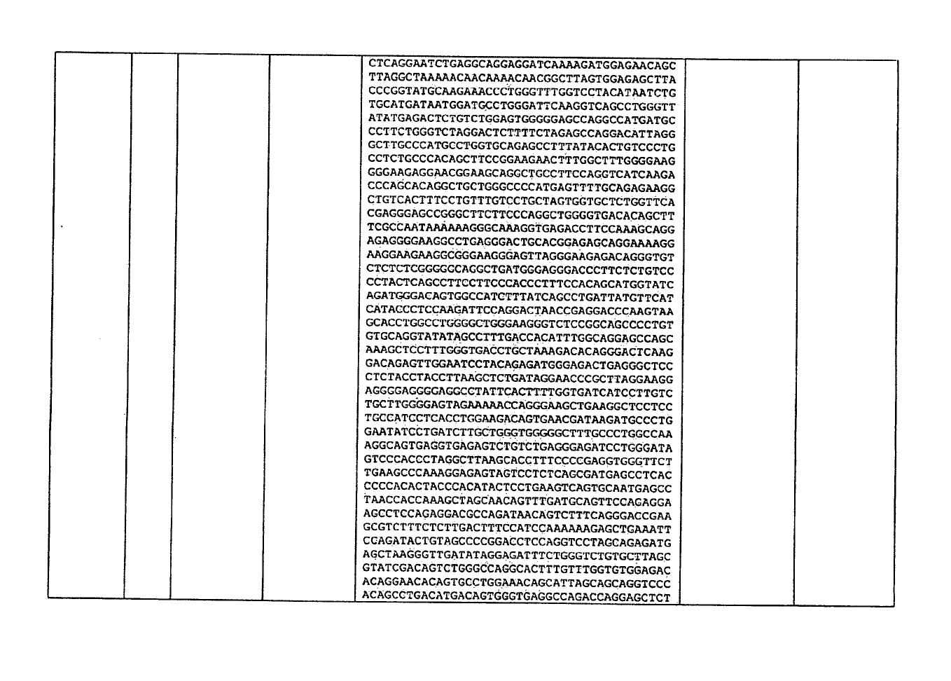

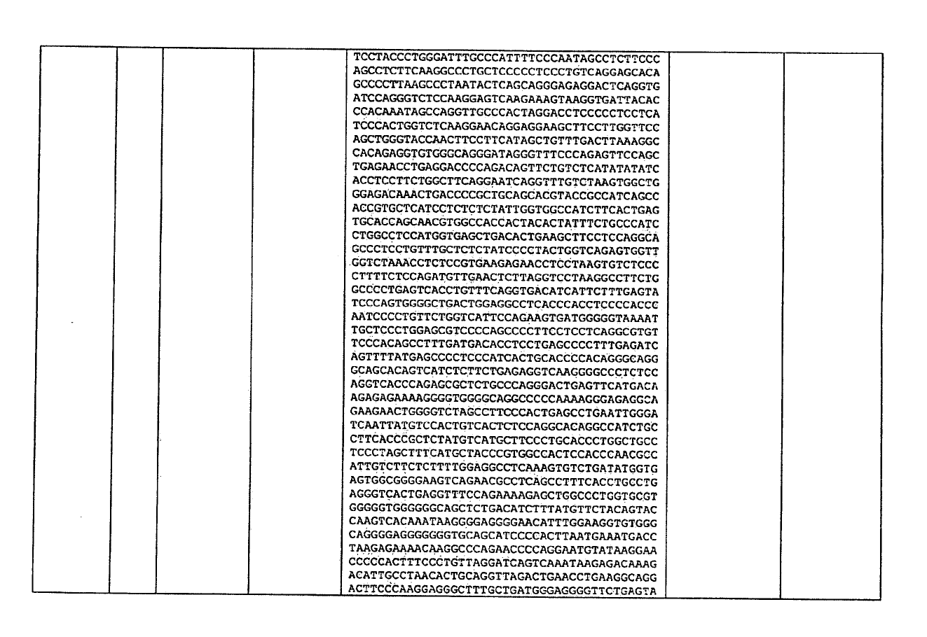

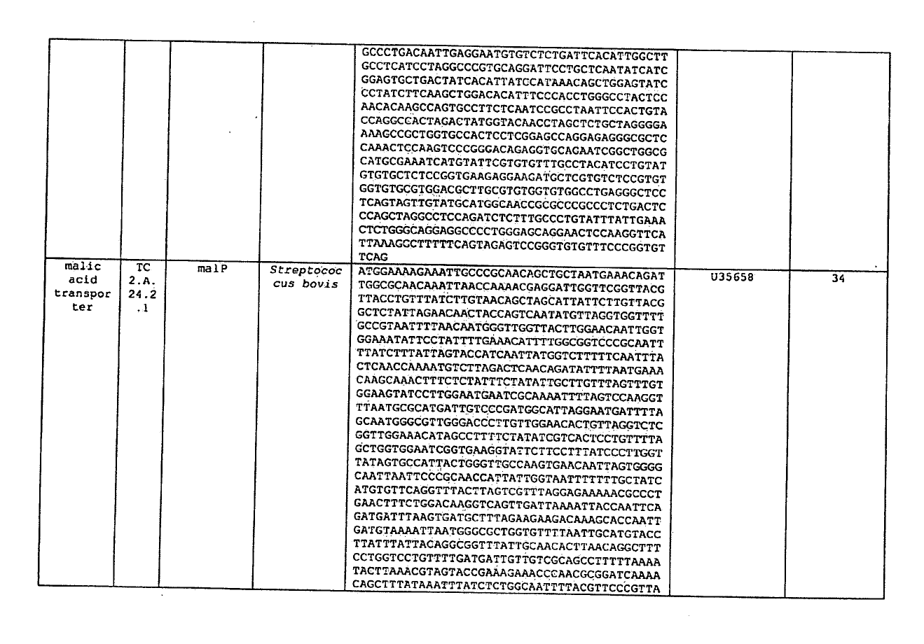

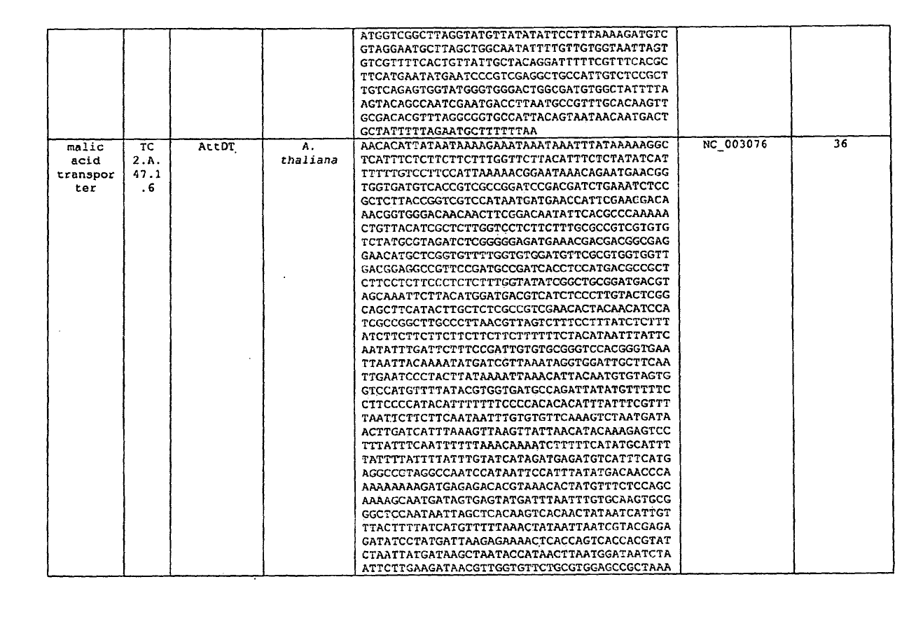

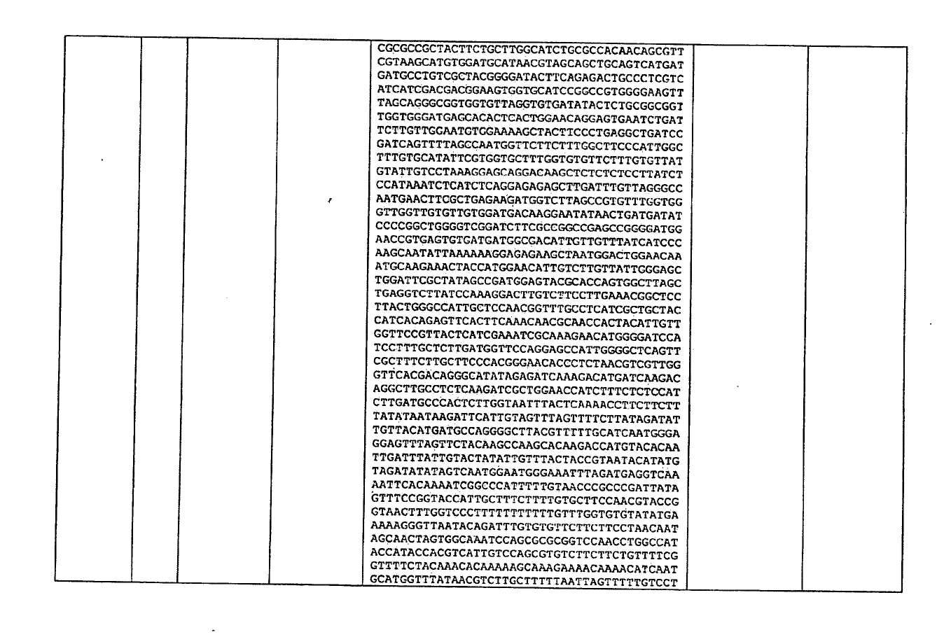

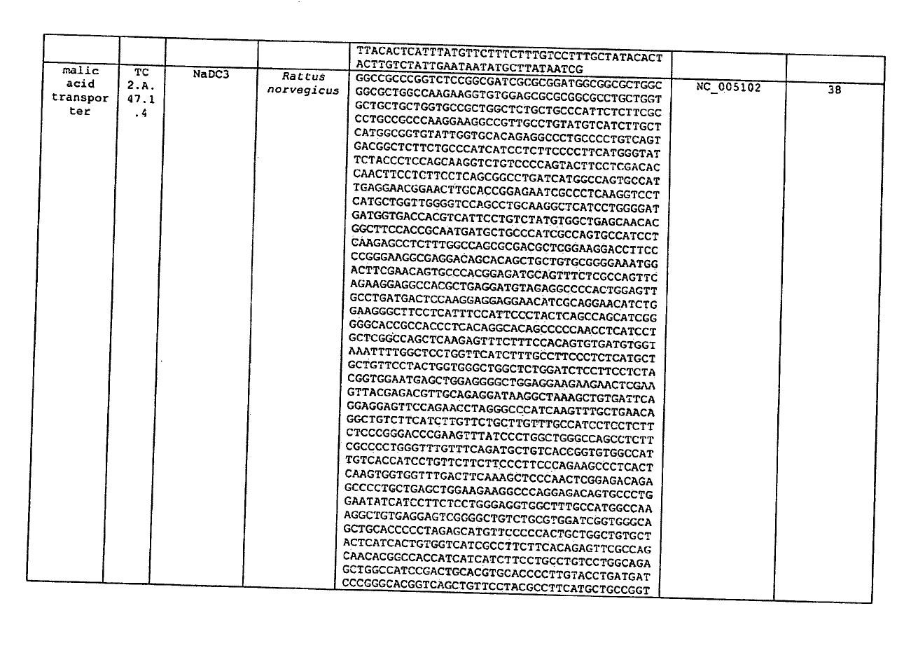

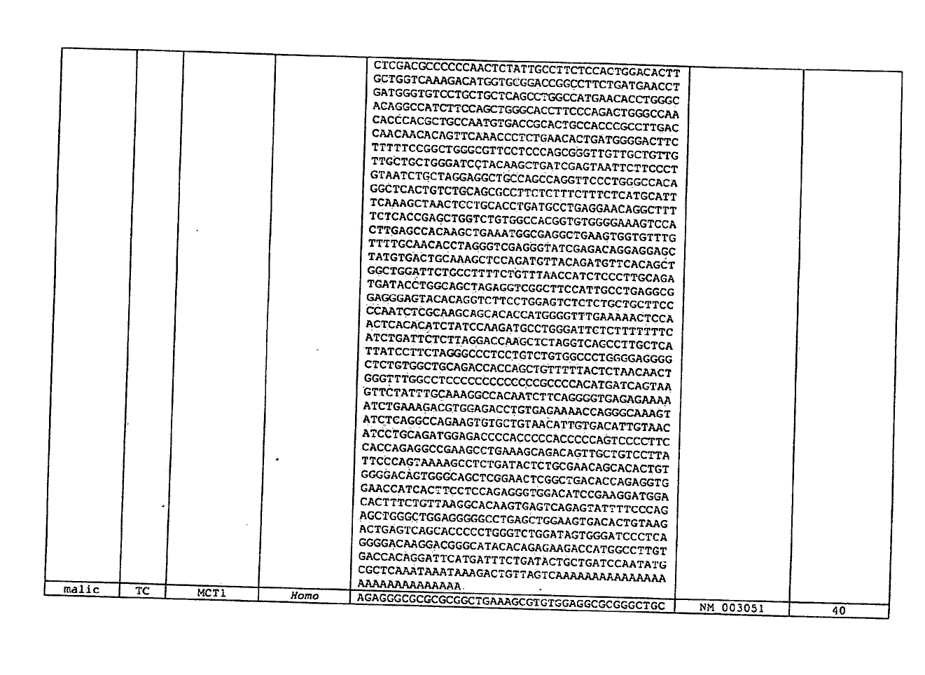

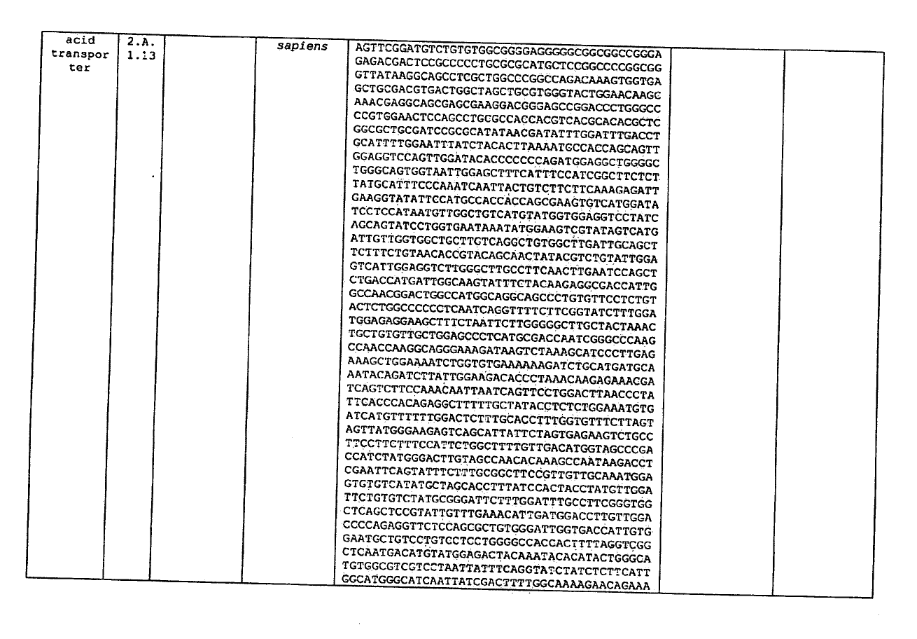

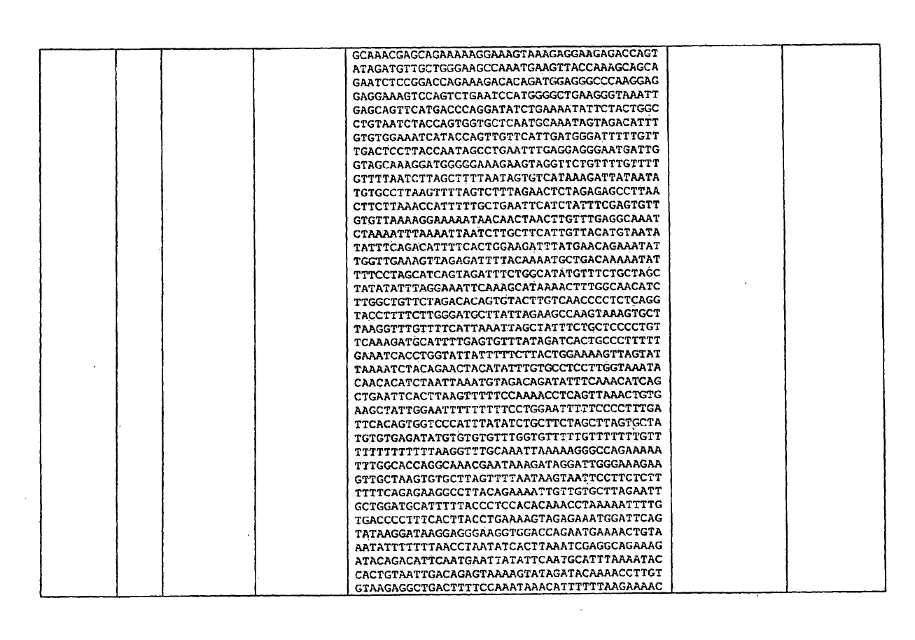

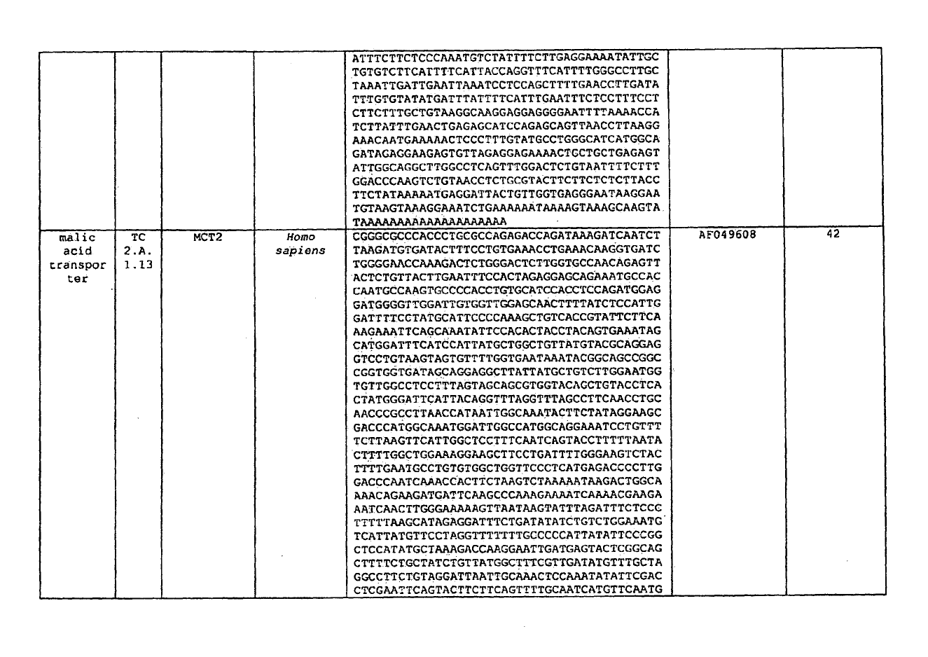

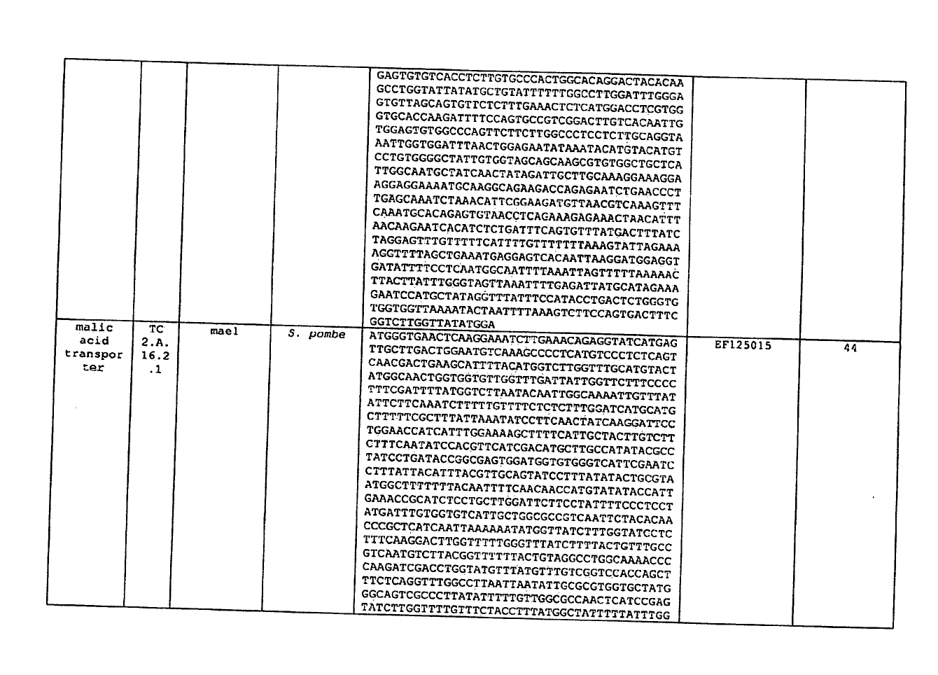

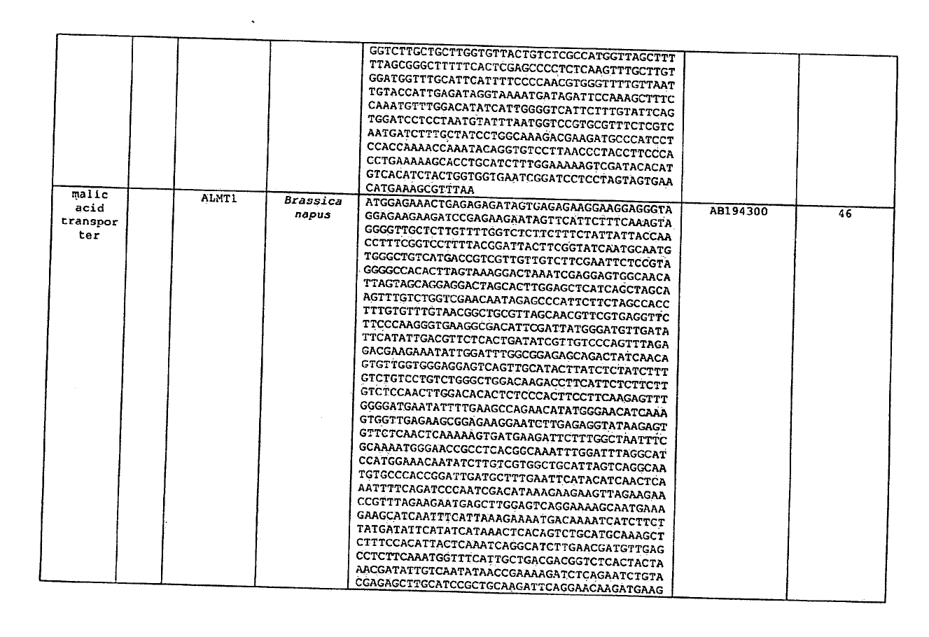

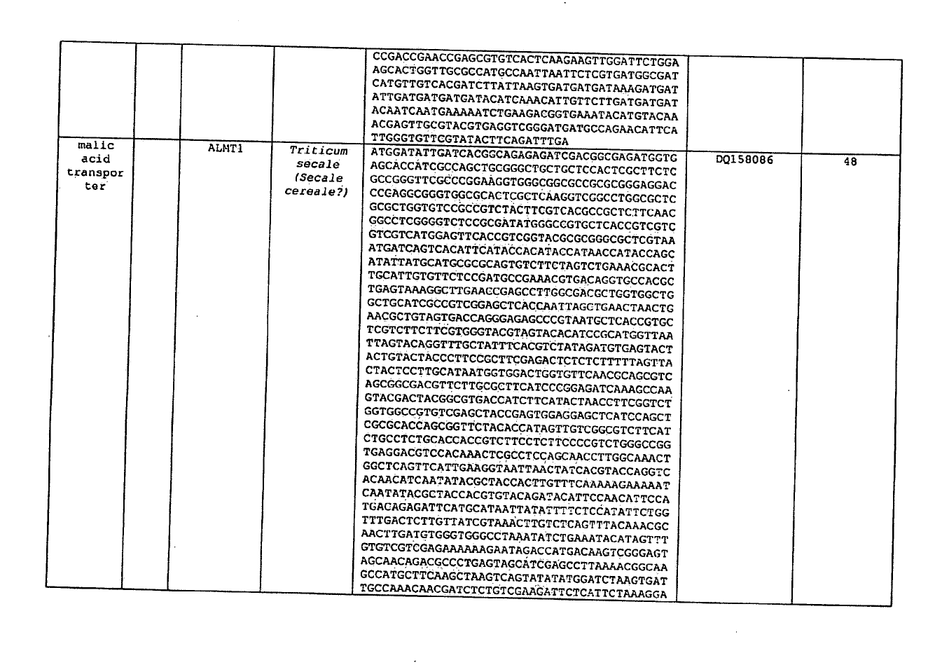

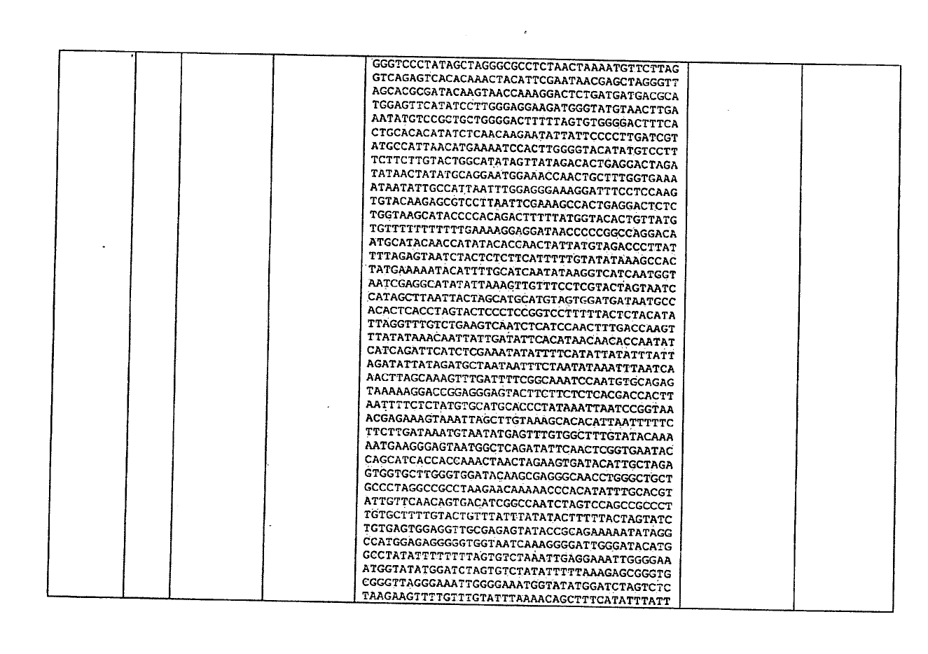

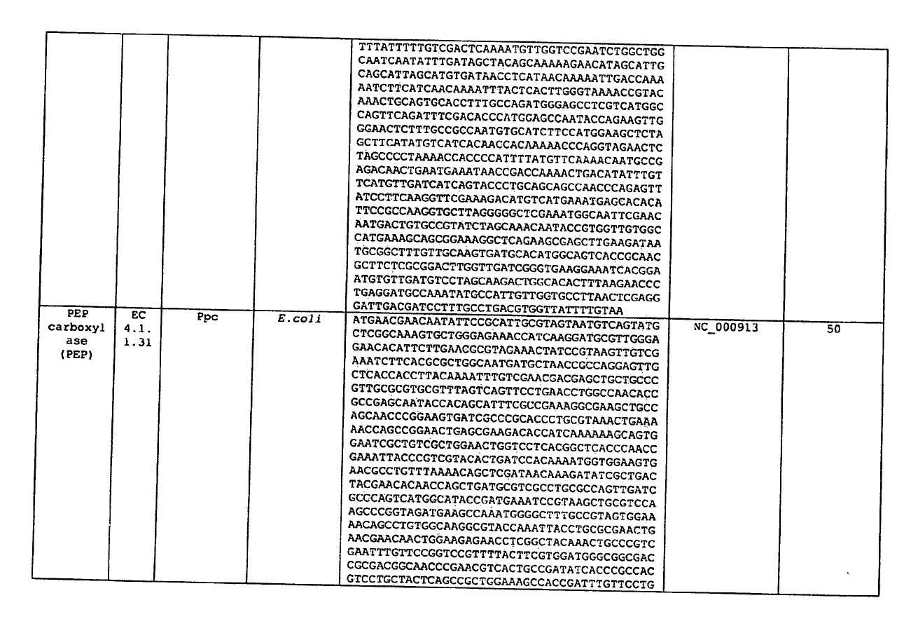

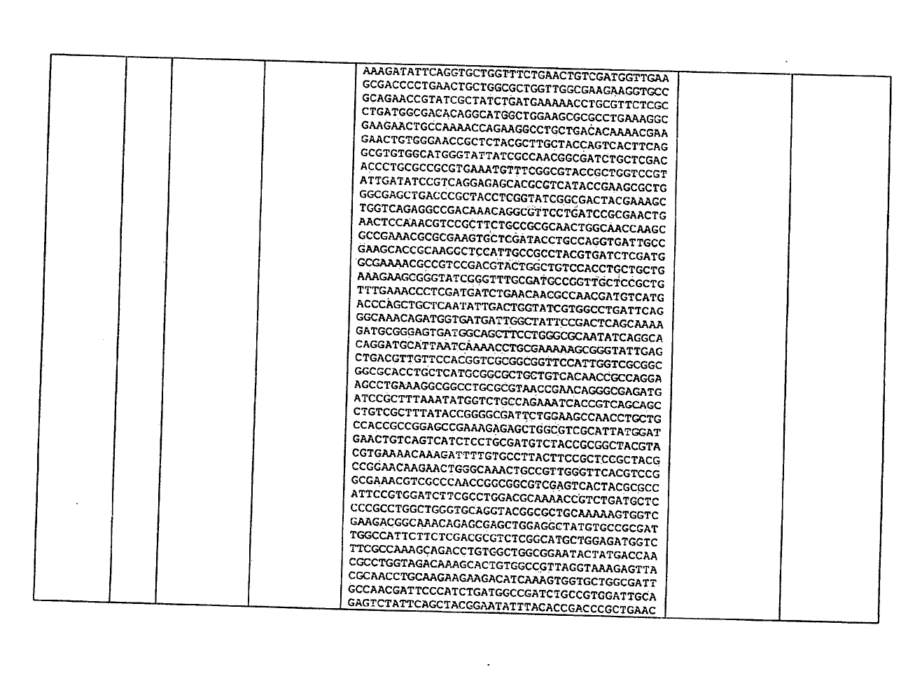

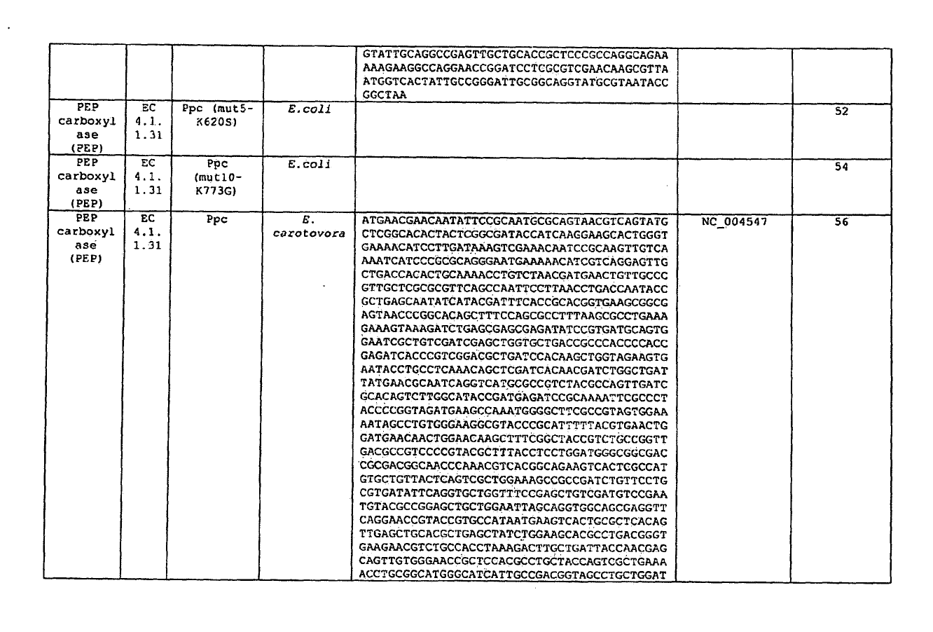

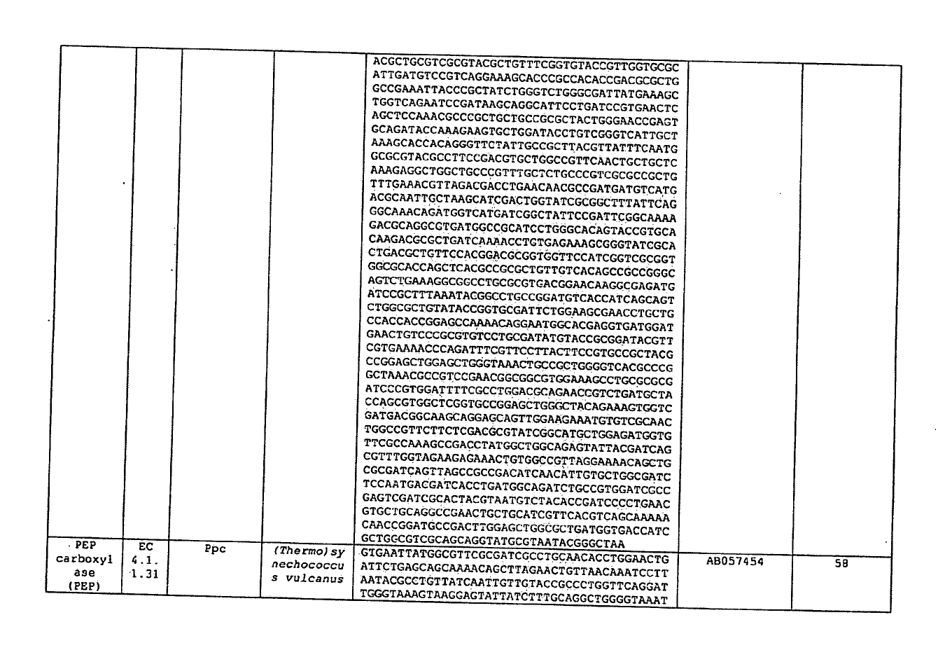

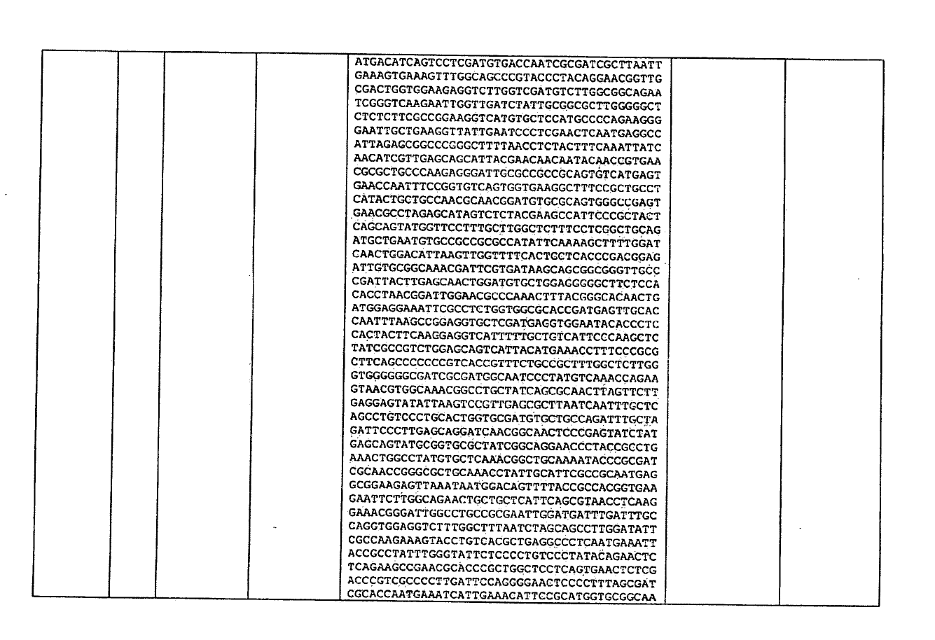

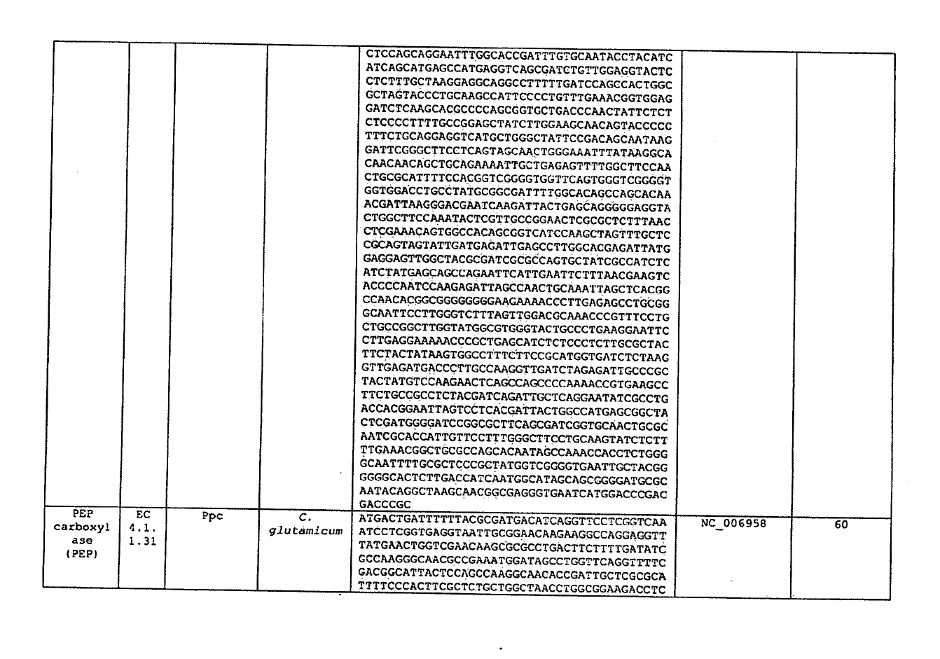

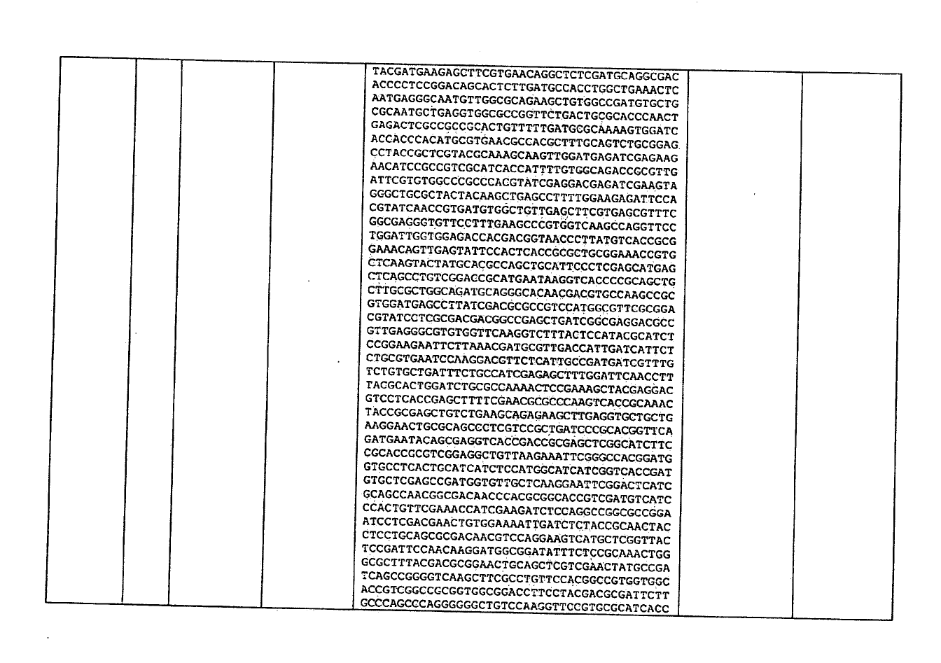

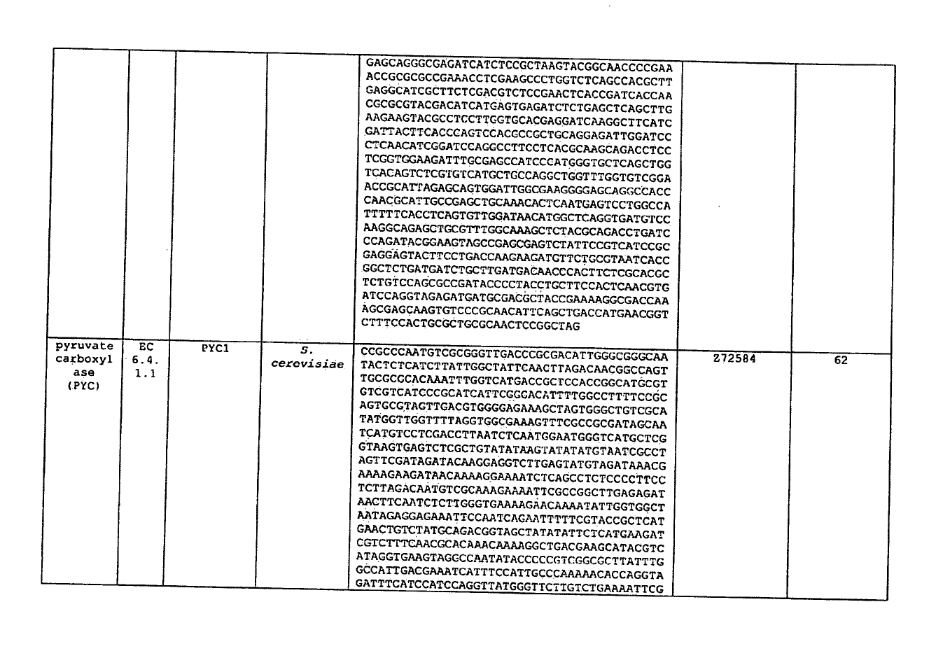

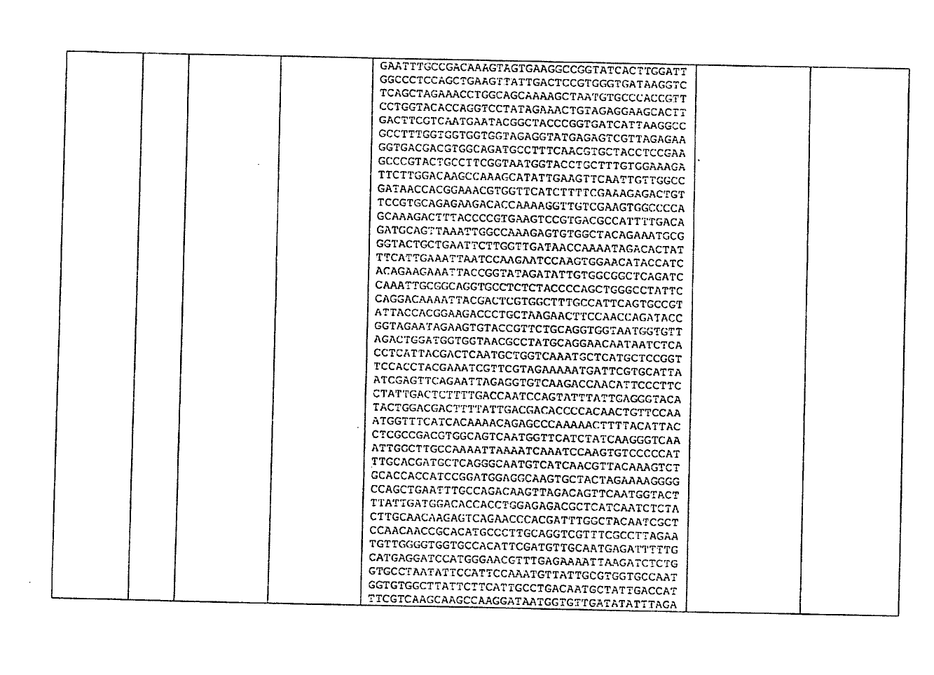

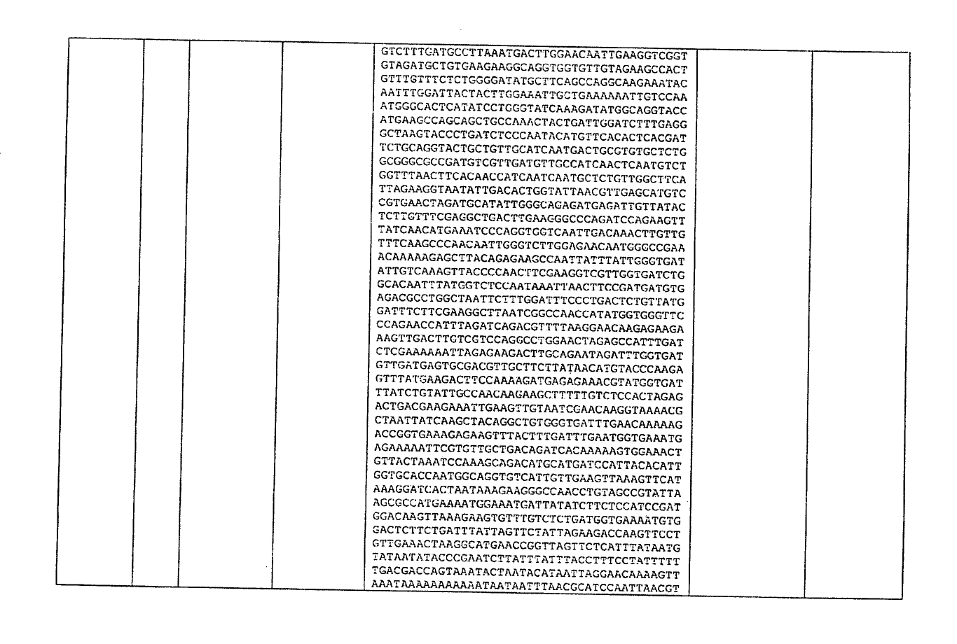

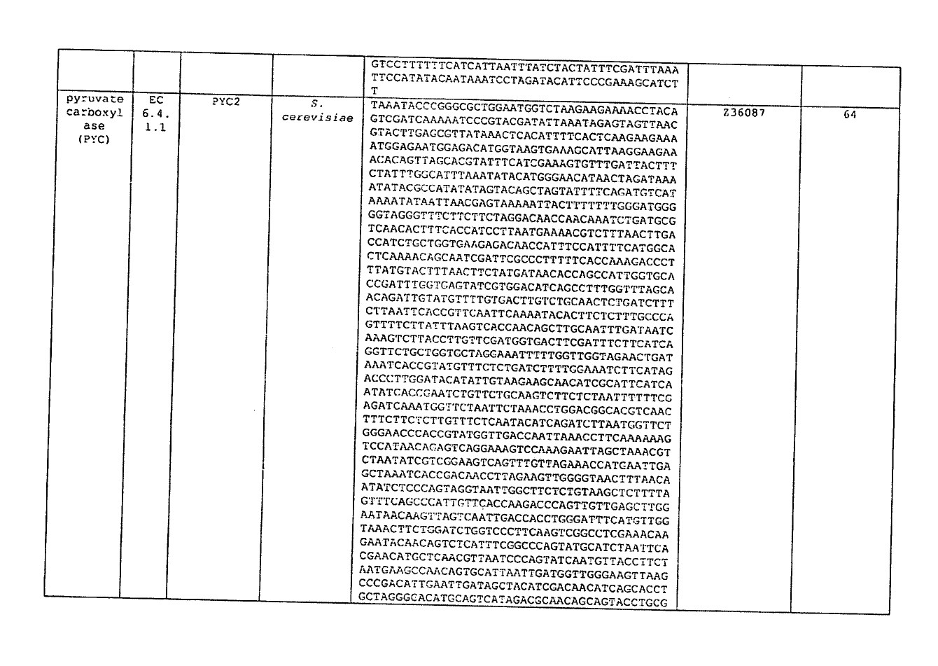

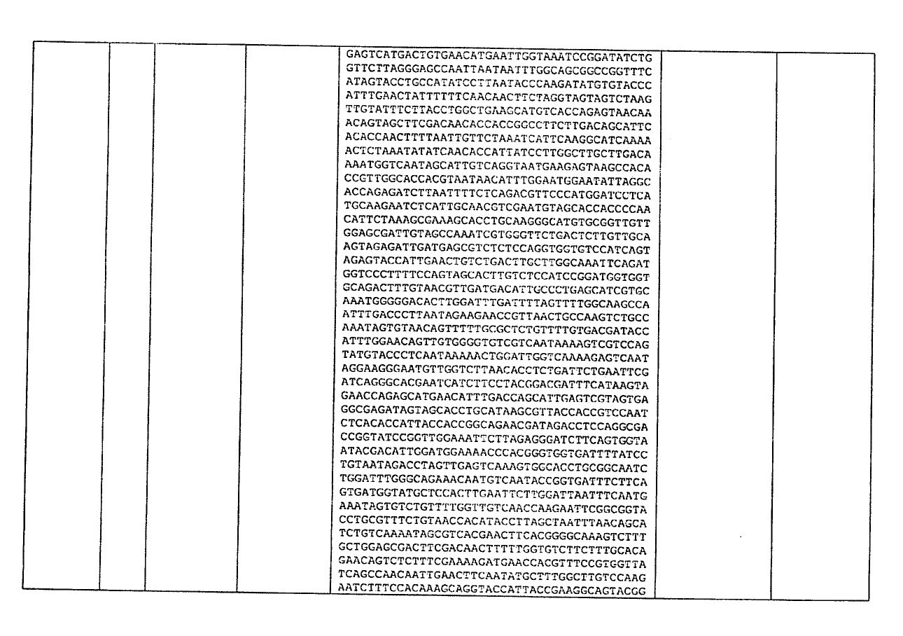

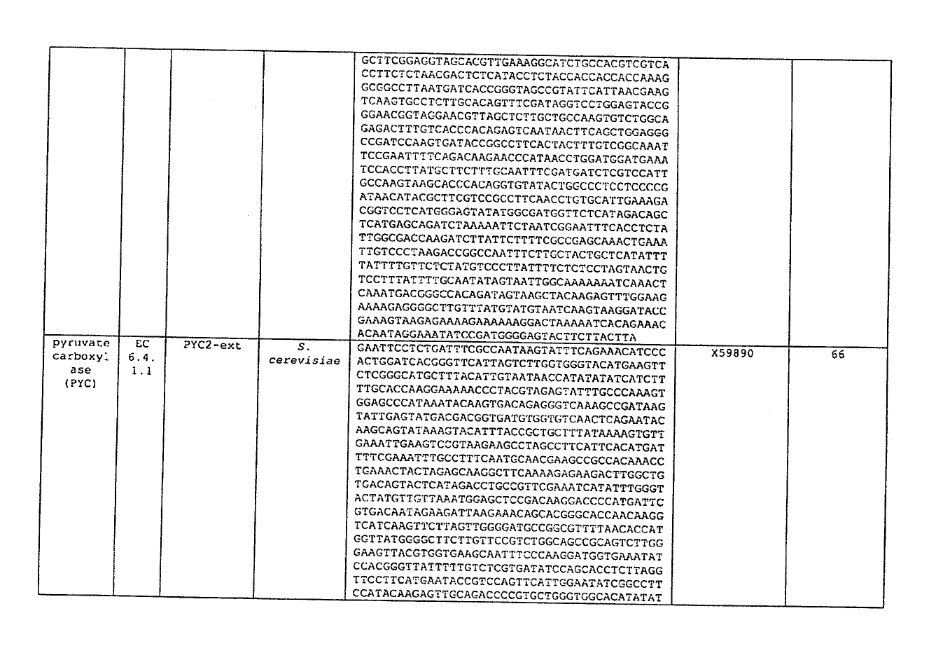

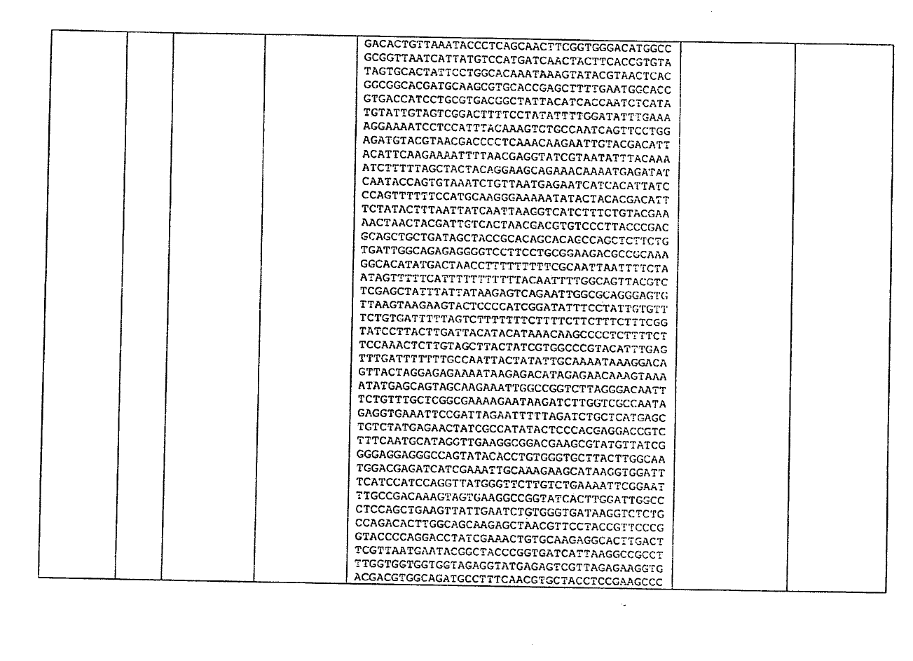

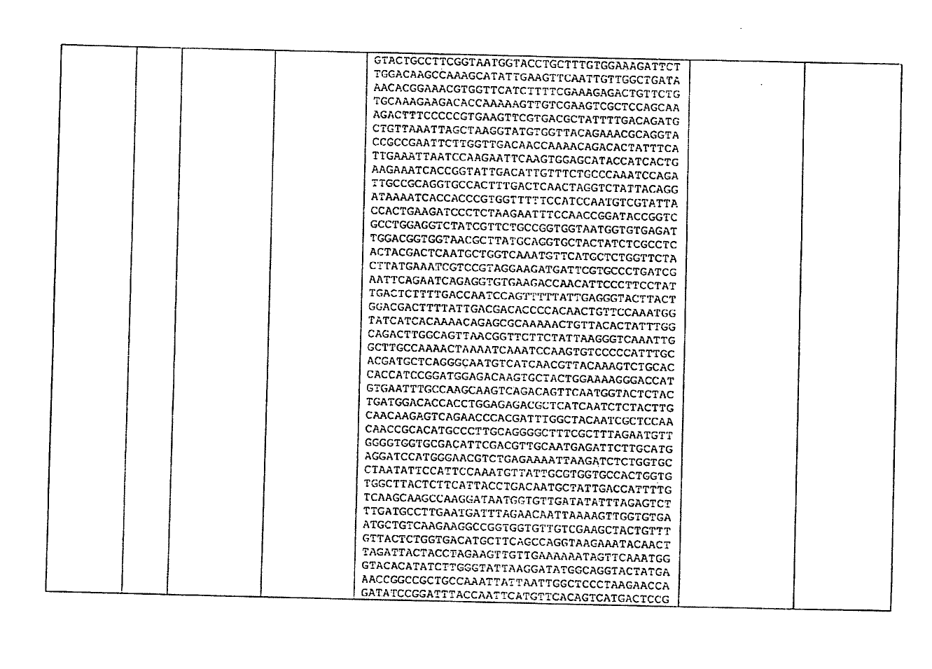

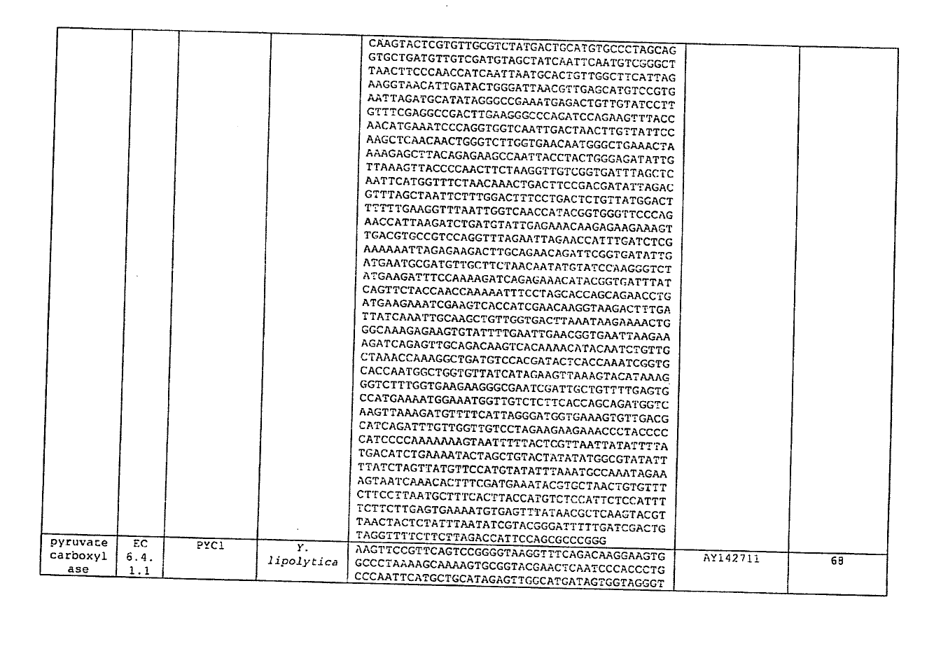

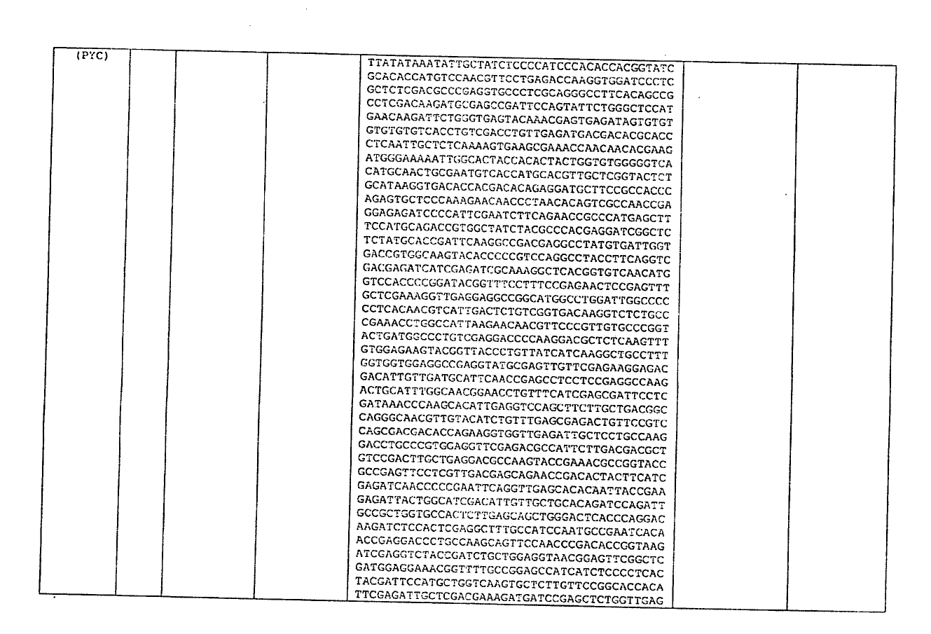

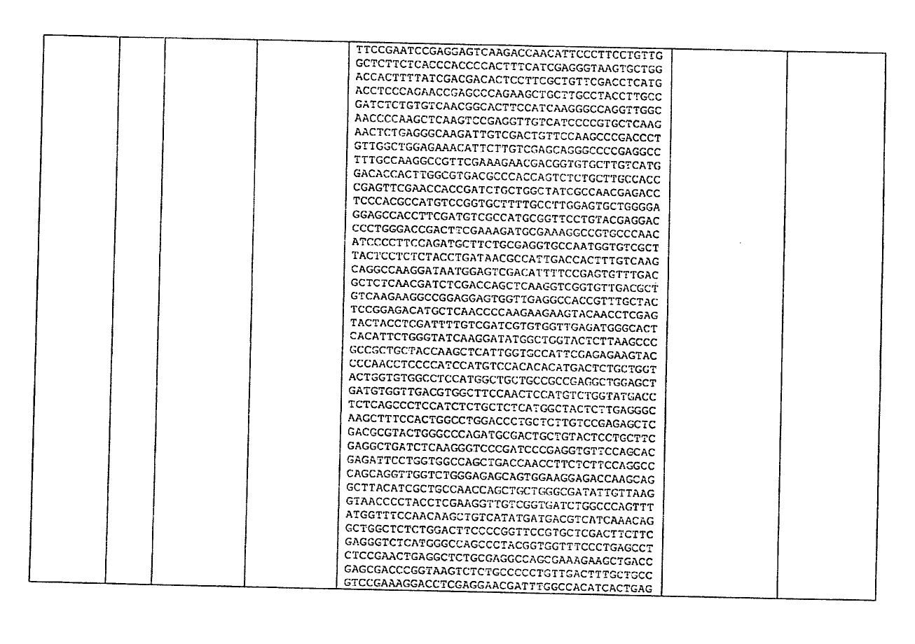

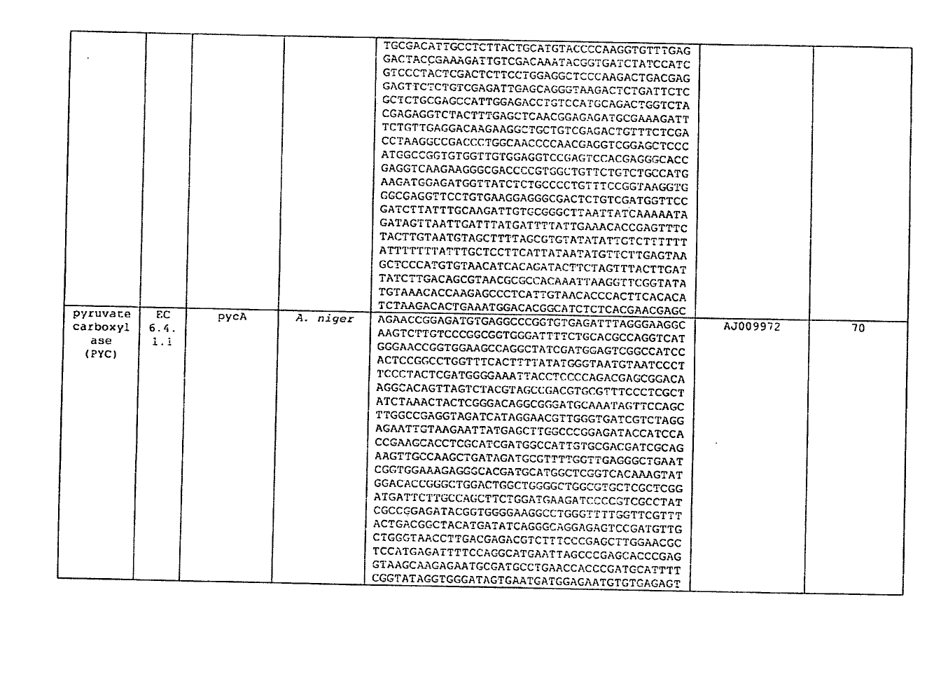

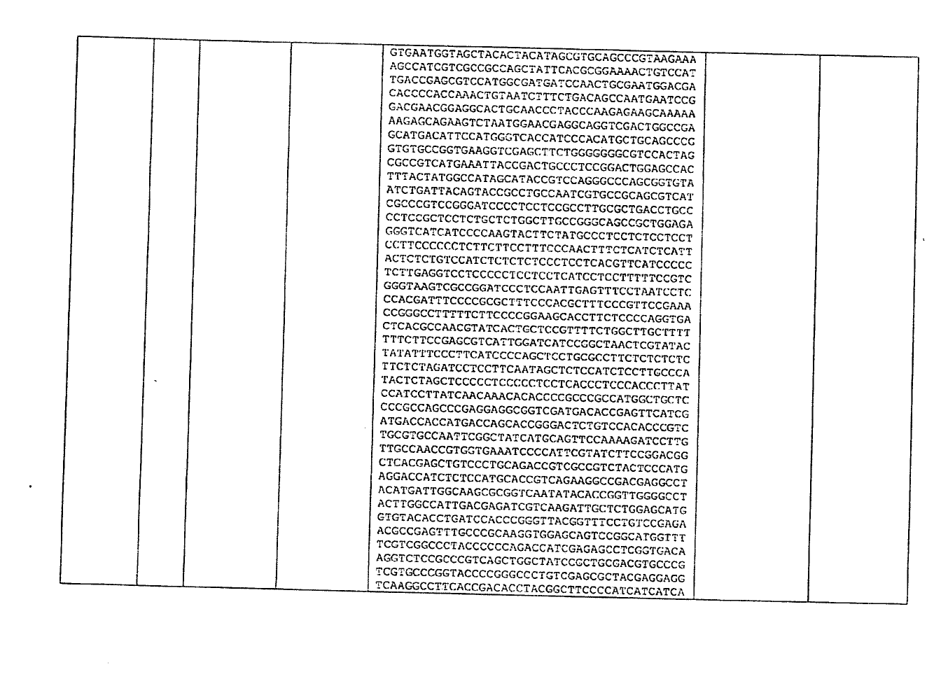

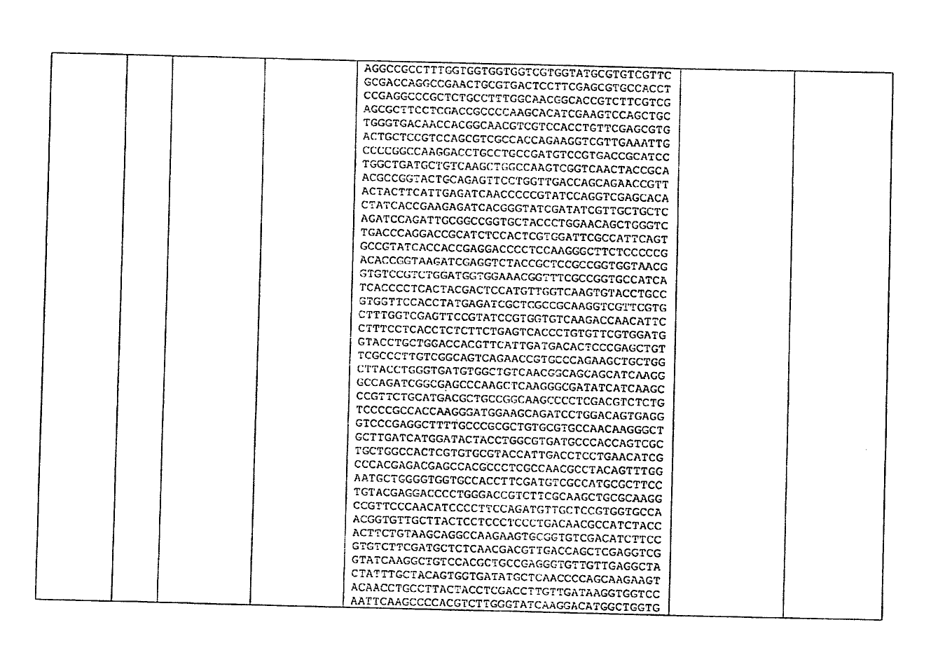

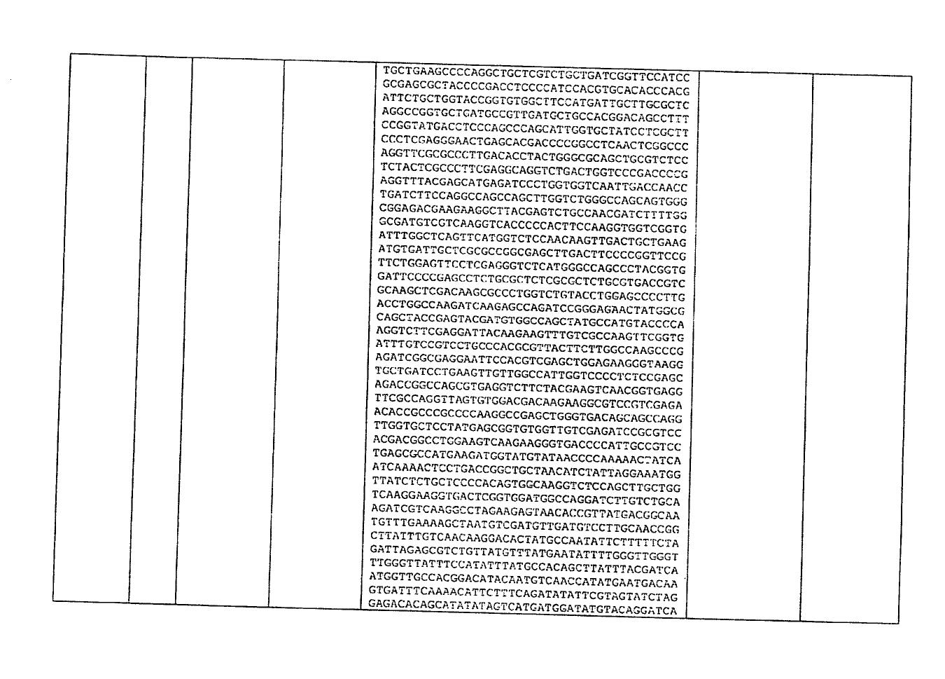

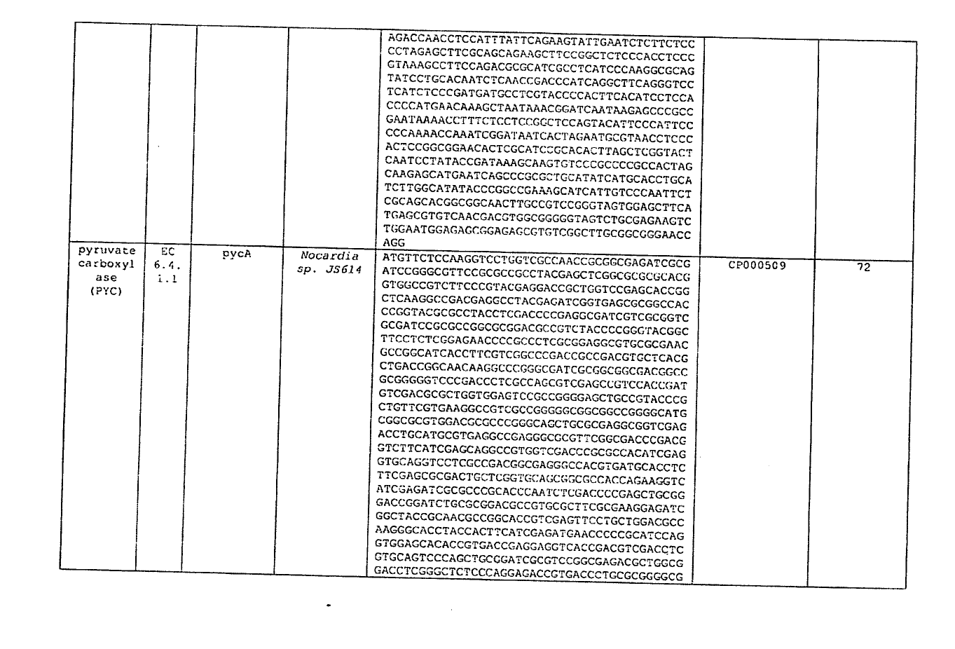

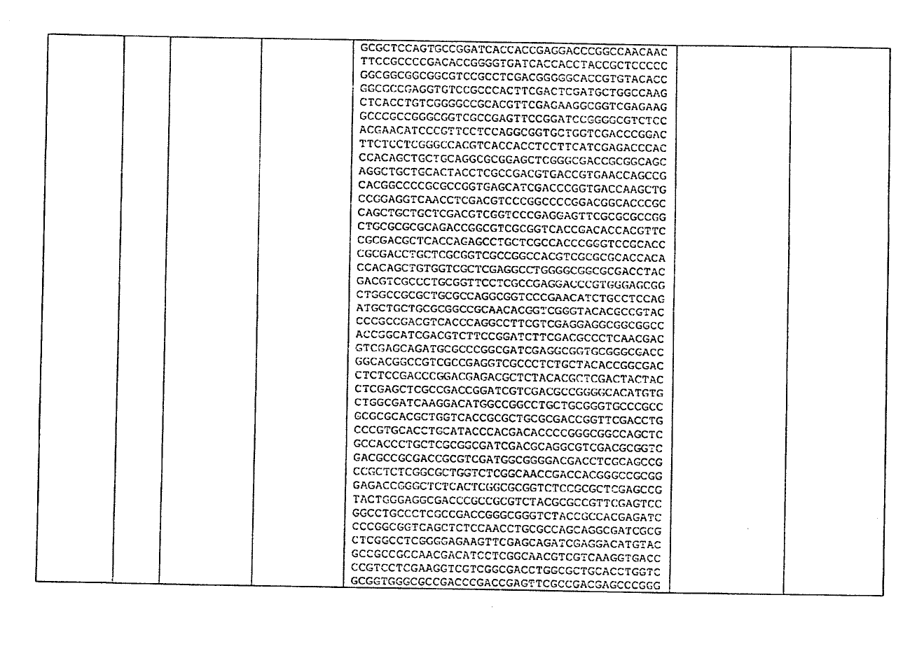

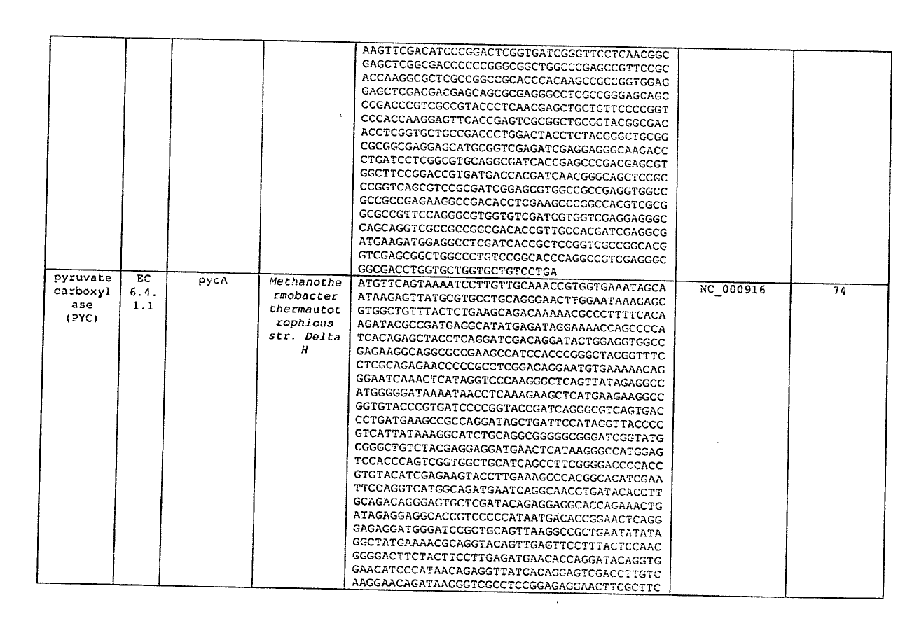

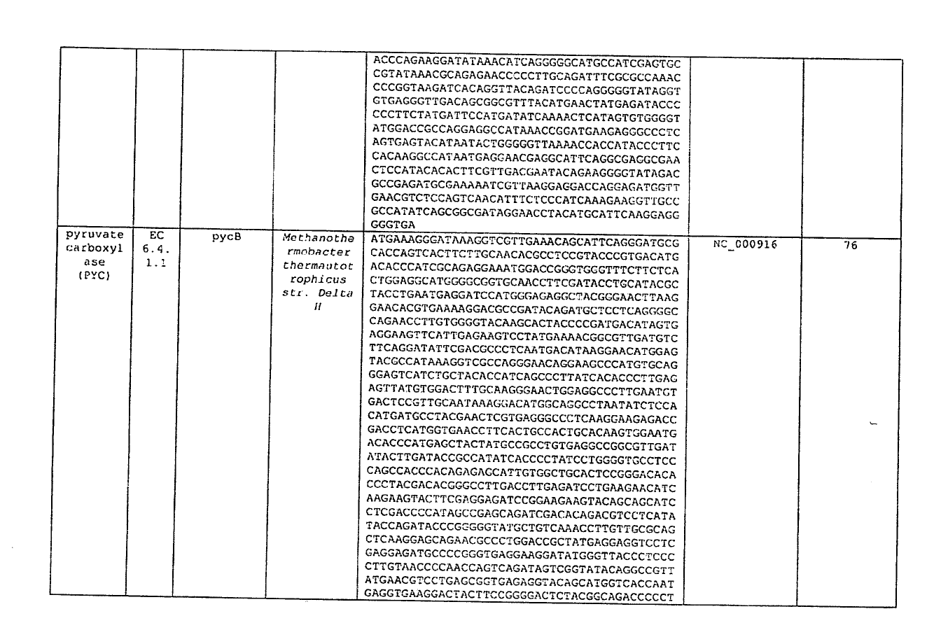

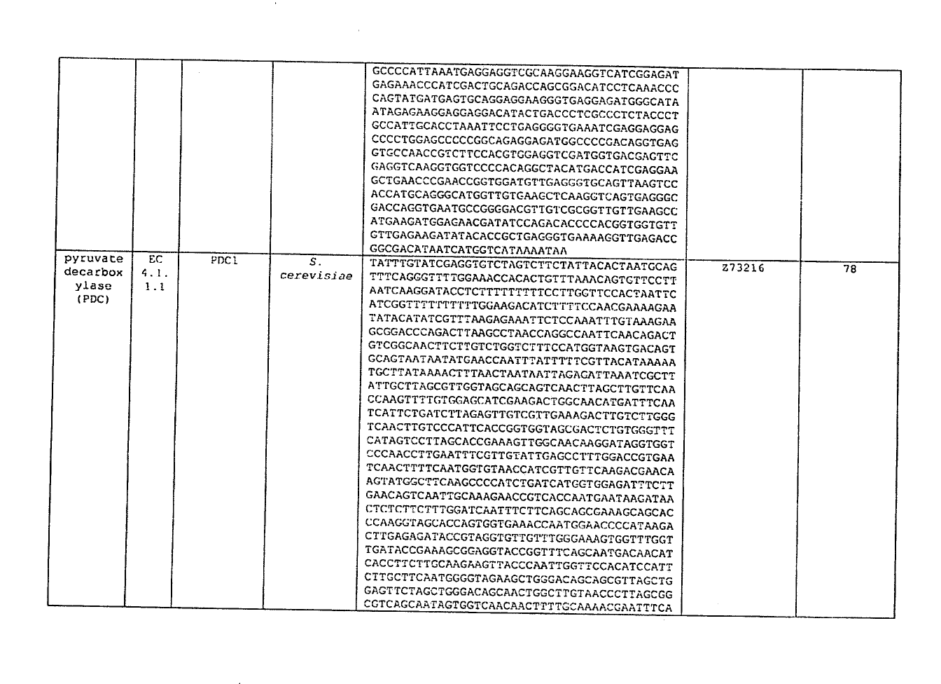

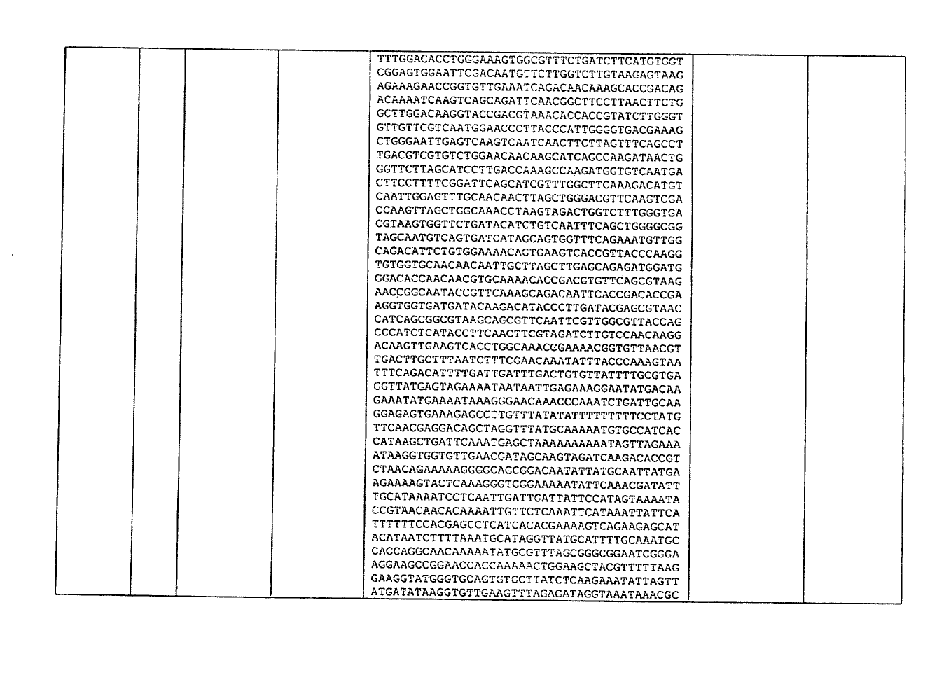

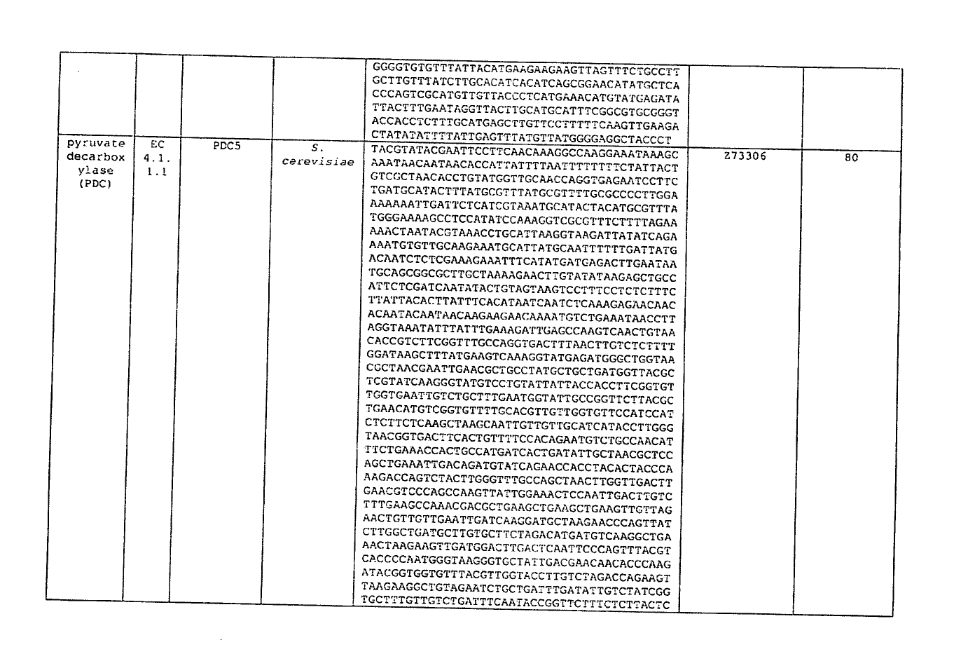

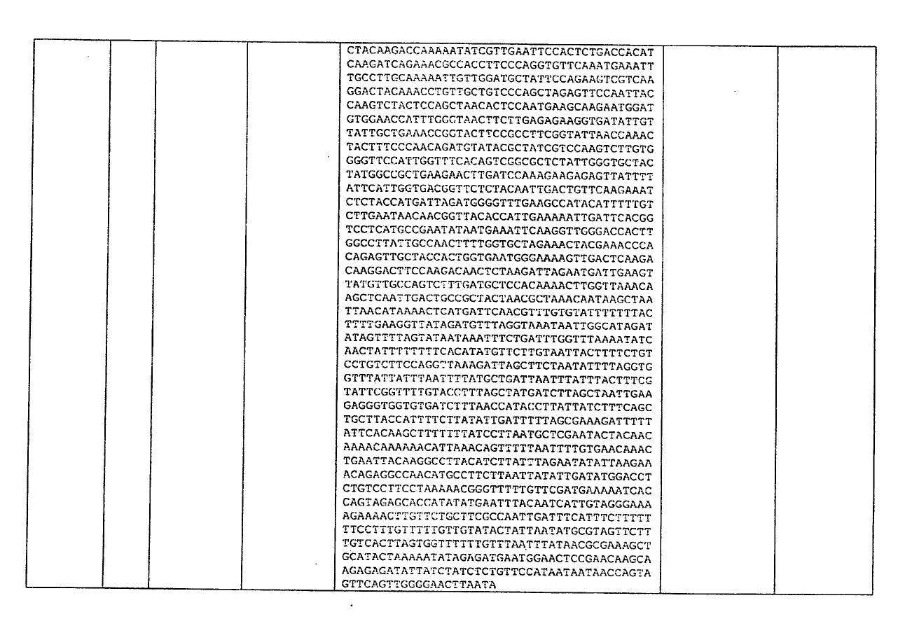

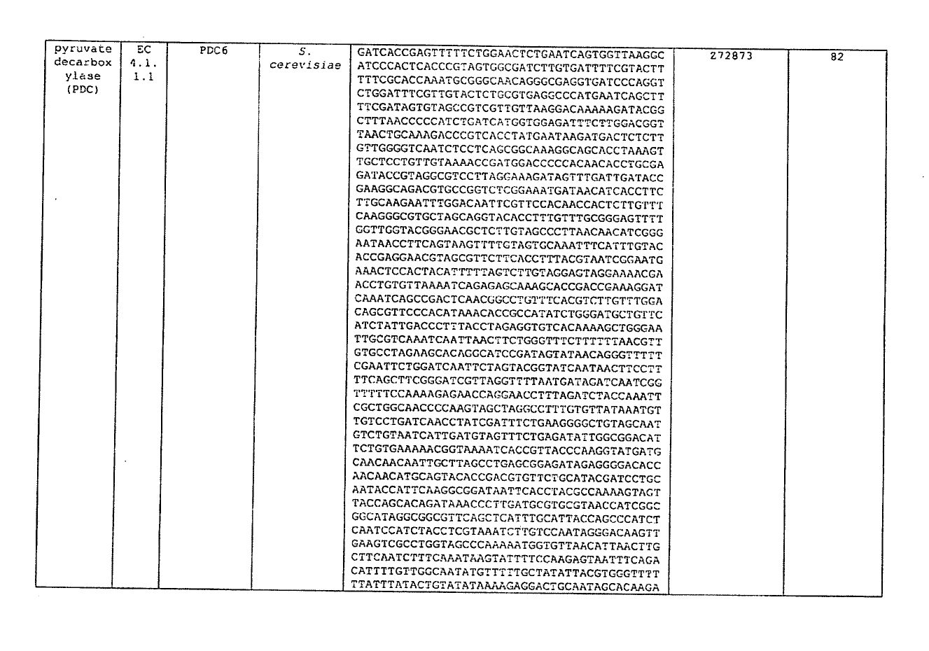

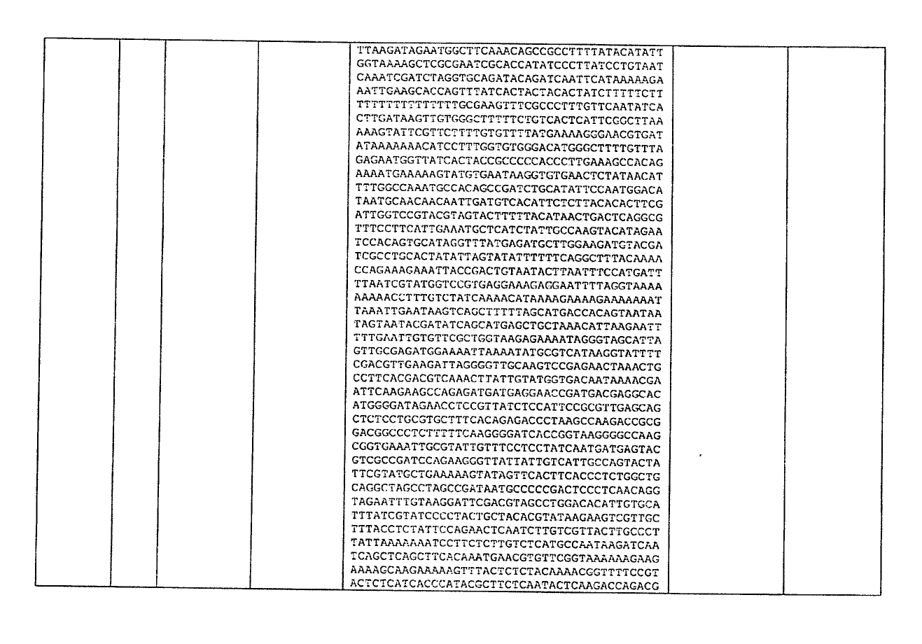

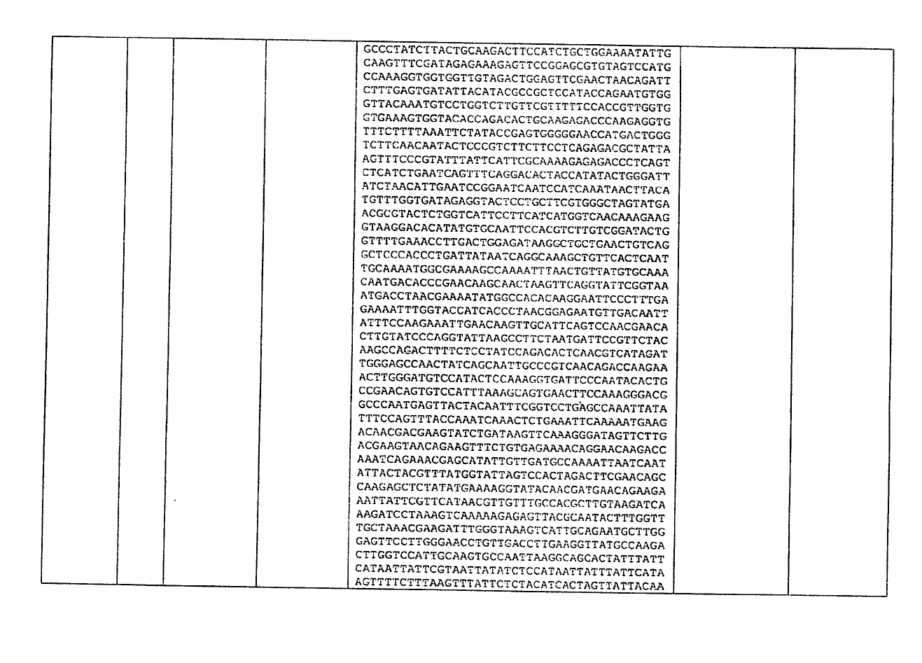

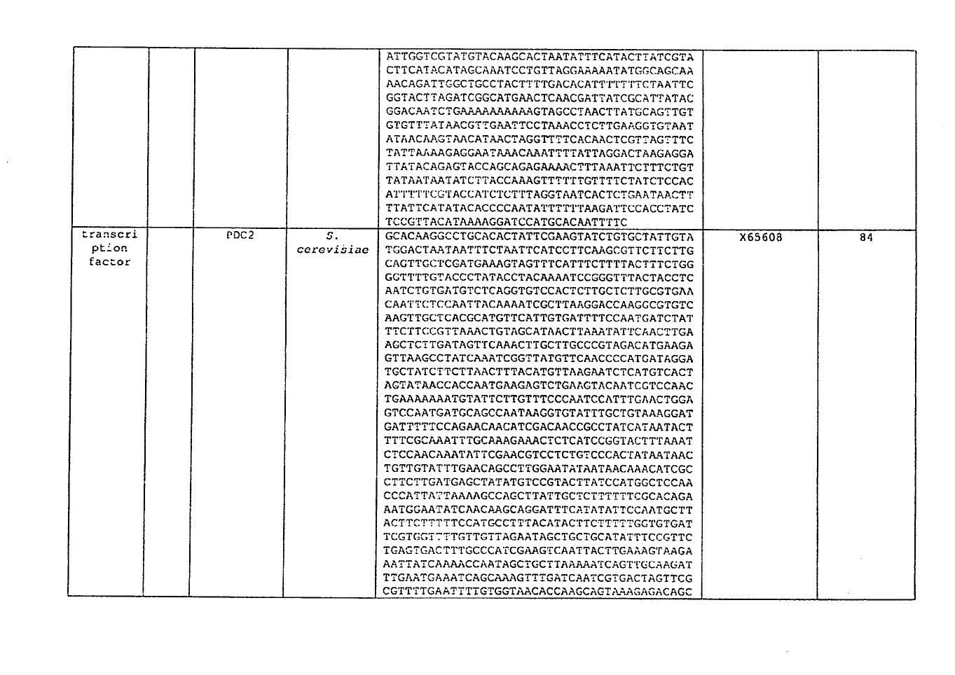

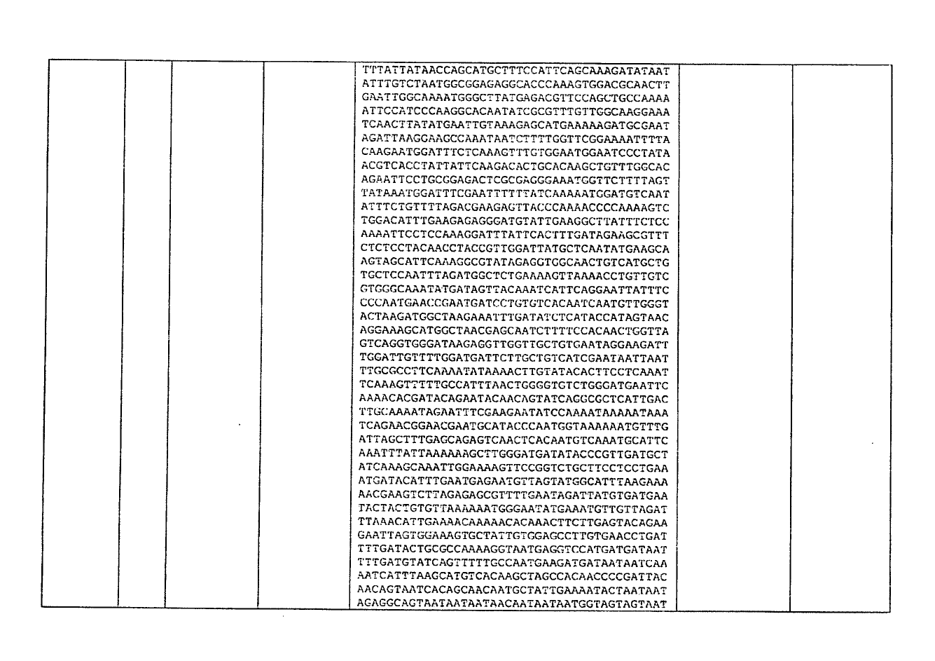

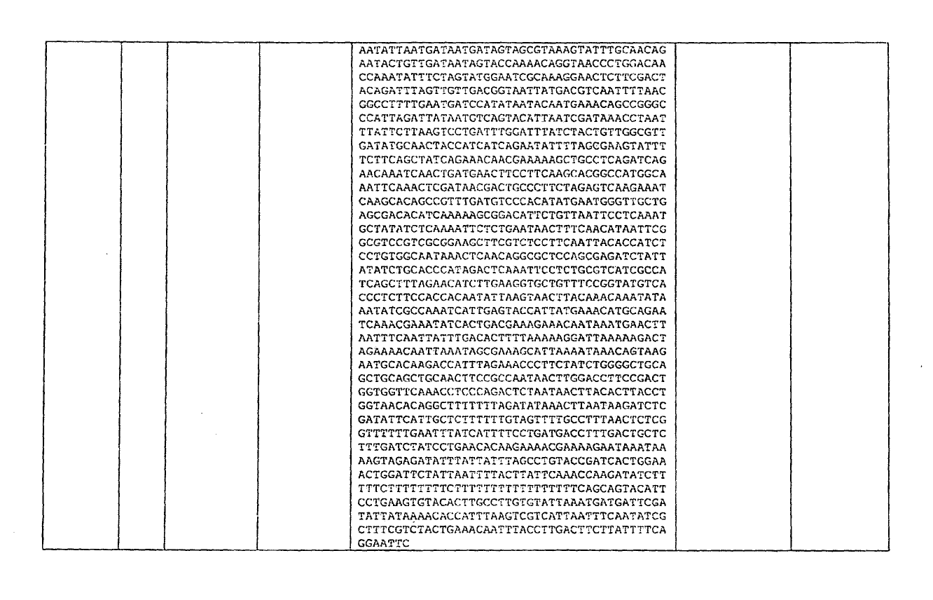

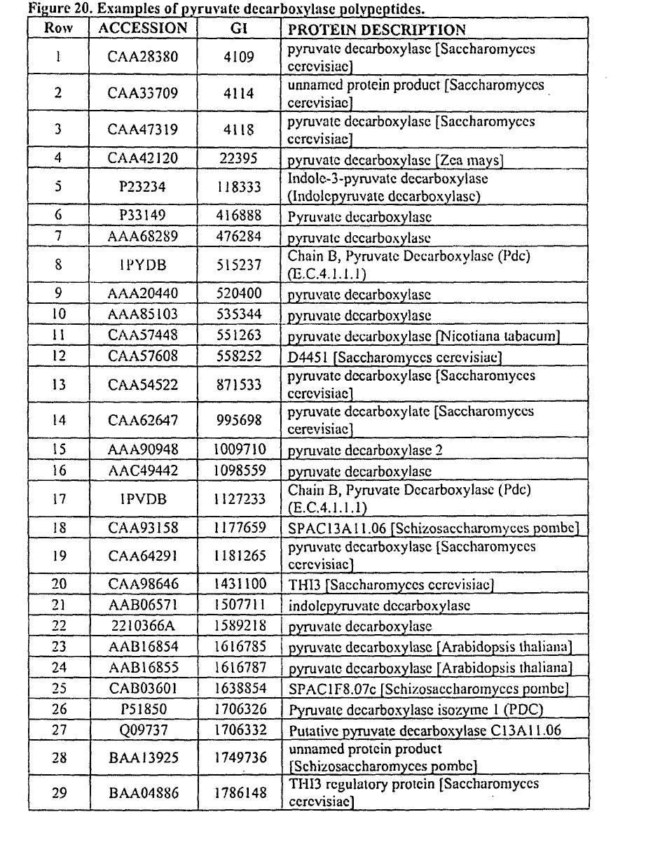

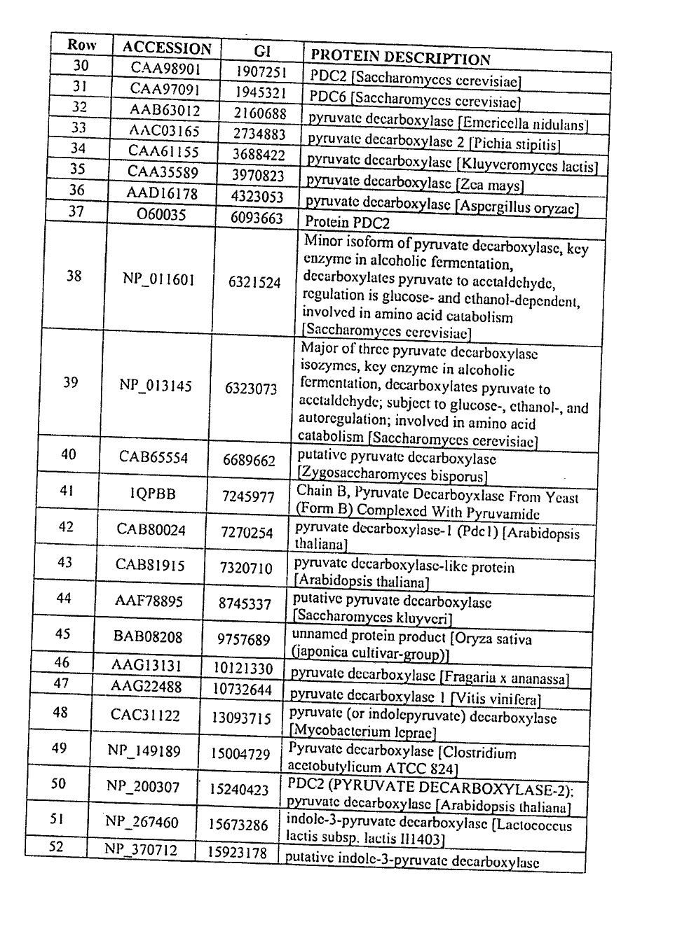

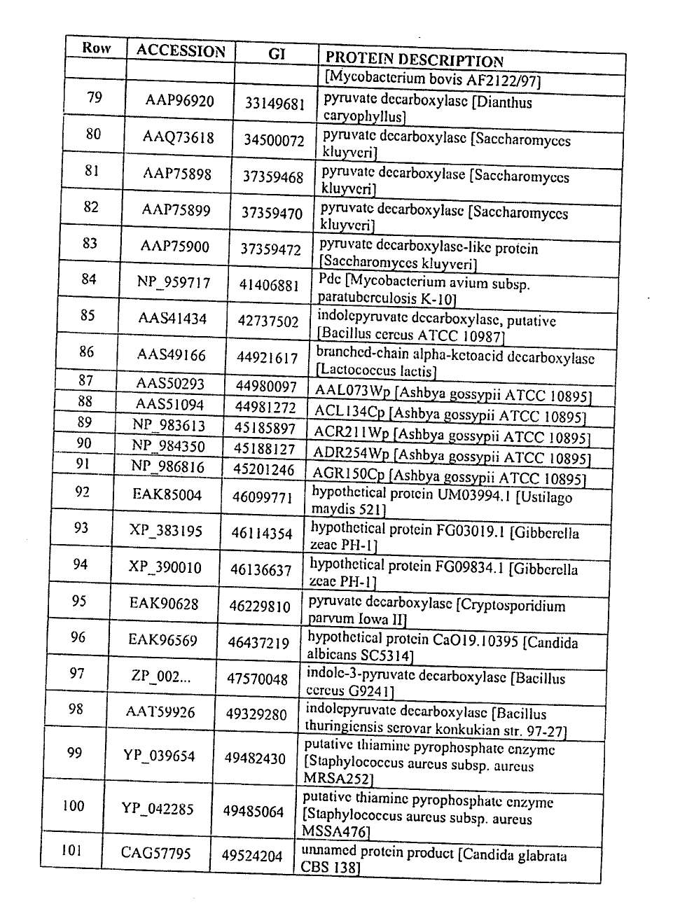

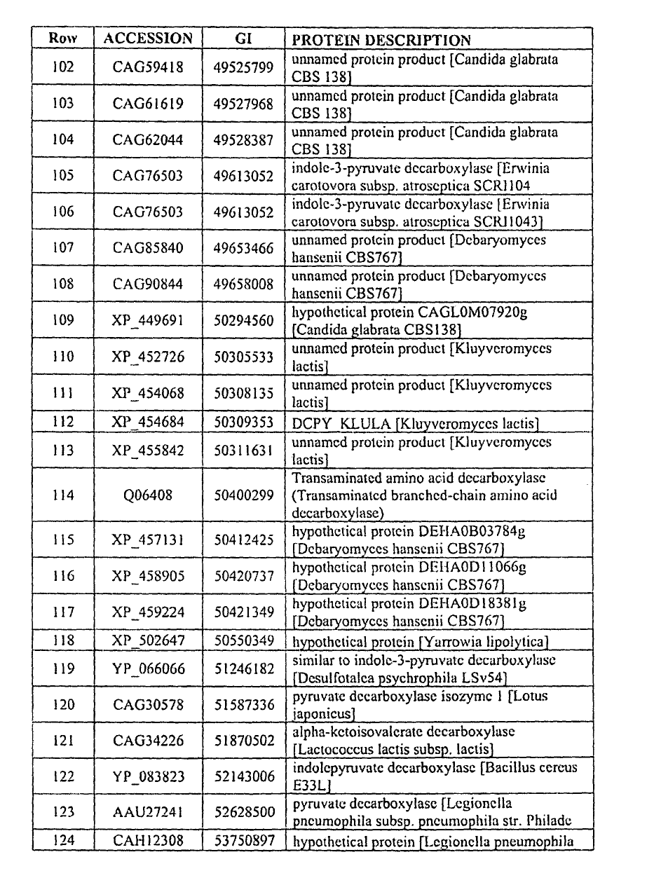

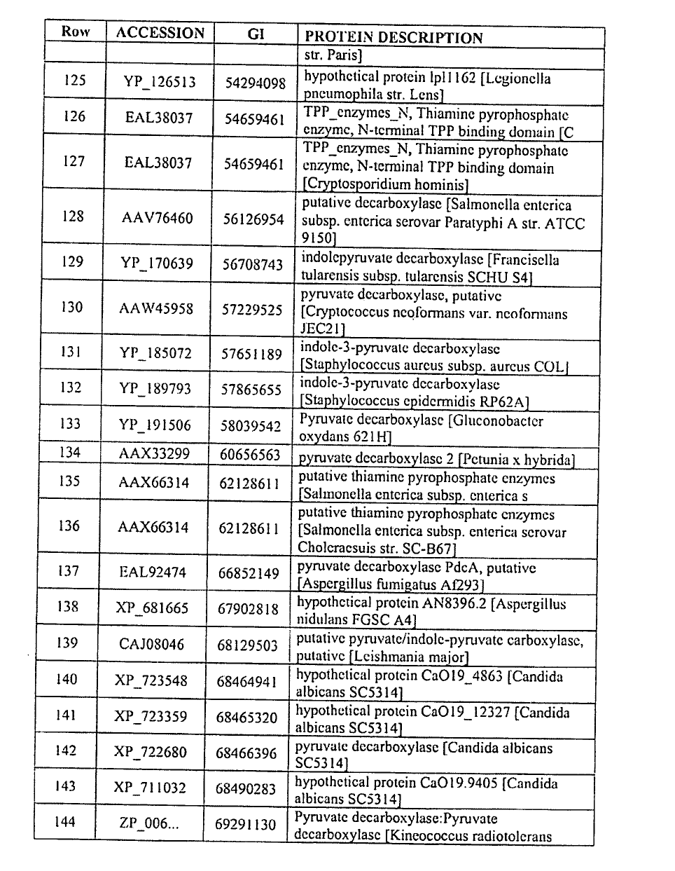

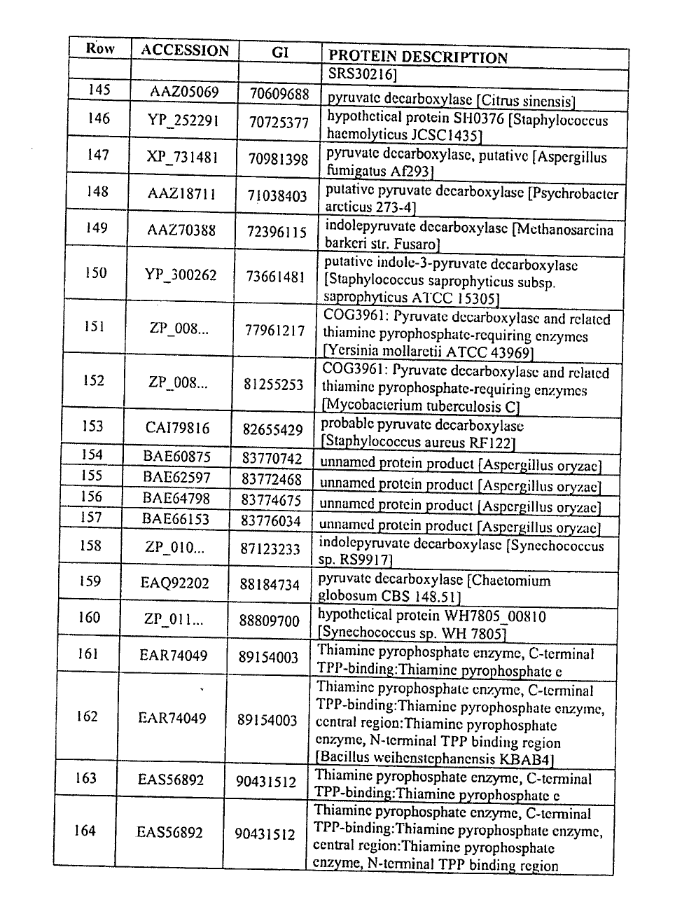

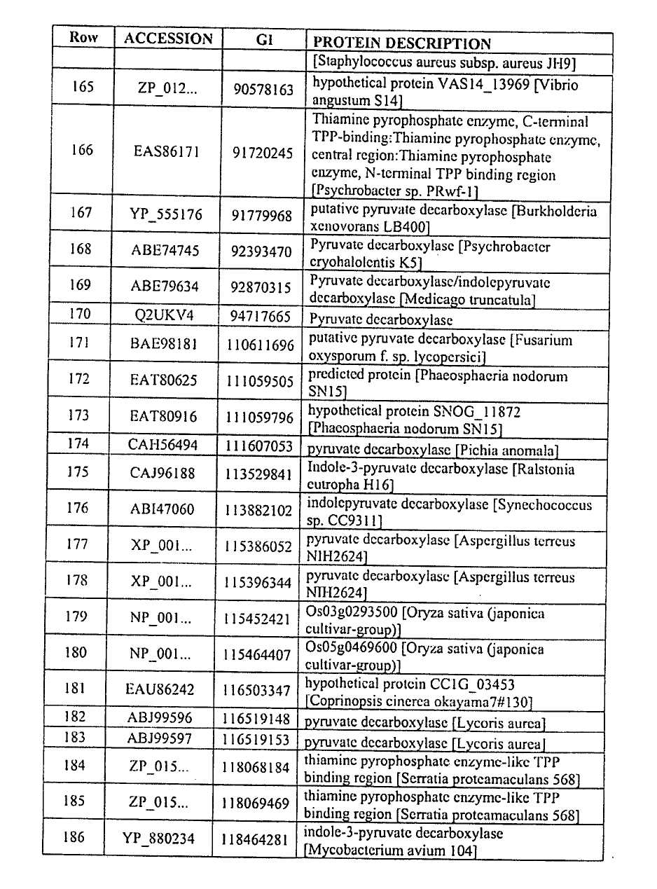

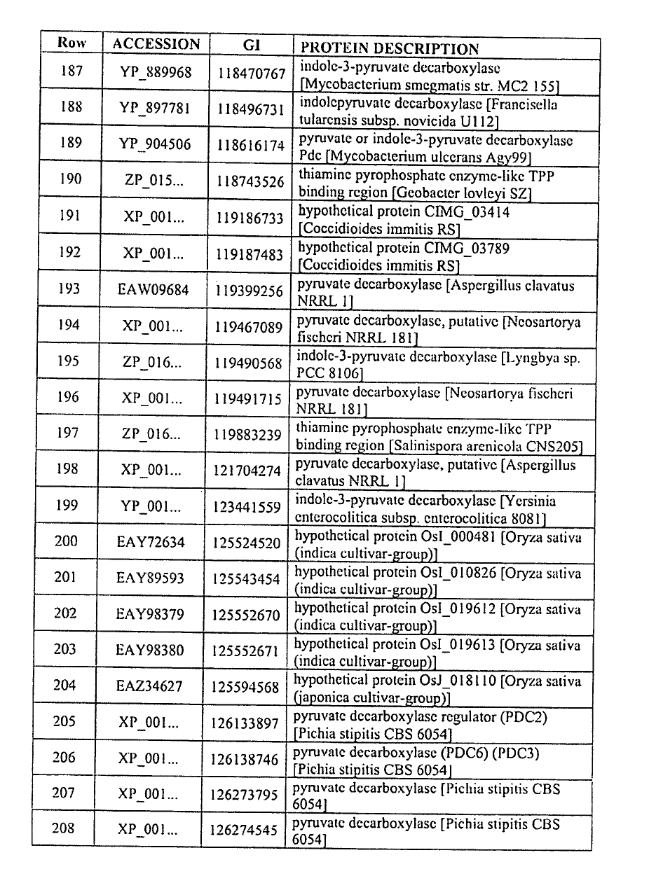

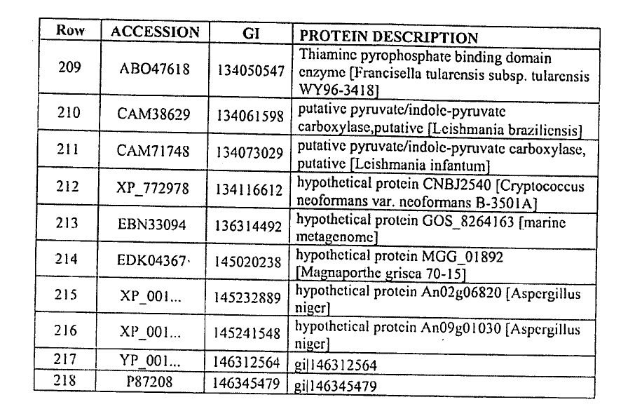

Concerning the PDC, PYC, PPC, MDH, and MAE polypeptides, the skilled artisan having the benefit of the present disclosure will understand, in light of the redundancy of the genetic code, that a large number of potential coding regions can exist which will encode a particular PDC polypeptide sequence, PYC polypeptide sequence, PPC polypeptide sequence, MDH polypeptide sequence, or MAE polypeptide sequence. An exemplary PYC coding region is given as SEQ ID NO:4; an exemplary PPC coding region is given as SEQ ID NO:7 an exemplary MDH coding region is given as SEQ ID N0:5; and an exemplary MAE coding region is given as SEQ ID NO:6. Additional exemplary PDC, PYC, PPC, MDH and MAE coding regions are given in FIGS. 19 as those DNA sequences which encode pyruvate carboxylase, PPC, malate dehydrogenase and organic acid transport polypeptides. Any coding region which will encode a desired protein sequence may be used in accordance with the present disclosure. The skilled artisan will understand that particular codons (“biased codons”) may have larger corresponding tRNA pools in the yeast than different redundant codons and thus may allow more rapid protein translation in the yeast. Additional PDC, PYC, PPC, MDH, and MAE polypeptides are represented by the polypeptides in FIG. 18, polypeptides that have at least 99%, 98%, 97%, 96%, 95%, 94%, 93%, 92%, 91%, 90%, 85%, 80%, 75% identity to a polypeptide represented in FIG. 18, polypeptides represented by the Genbank GI numbers in FIGS. 20-24 and FIG. 26, and polypeptides that have at least 99%, 98%, 97%, 96%, 95%, 94%, 93%, 92%, 91%, 90%, 85%, 80%, 75% identity to a polypeptide represented by the Genbank GI numbers in FIGS. 20-24 and FIG. 26.

The skilled artisan will also understand that various regulatory sequences, such as promoters and enhancers, among others known in the art, can be used as a matter of routine experimentation in preparation and use of the functionally transformed yeast as described herein.

The present disclosure is not limited to the enzymes of the pathways known for the production of malic acid intermediates or malic acid and/or succinic acid intermediates or succinic acid in plants, yeast, or other organisms.

Methods of Producing Malic Acid

In certain embodiments, the the present disclosure relates to a method of producing malic acid or succinic acid including culturing a modified (e.g., recombinant) yeast, wherein the yeast has reduced pyruvate decarboxylase enzyme (PDC) activity (i.e., is PDC-reduced or PDC-negative) and is functionally transformed to increase the activity of either a pyruvate'carboxylase (PYC) polypeptide, a phosphoenolpyruvate carboxylase (PPC) polypeptide, a malate dehydrogenase (MDH) polypeptide, and/or an organic acid transport (MAE) polypeptide. FIG. 25a-e depicts various organic acid biosynthetic pathways related to the production of malic acid. In most instances, the preferred pathway for malic acid or succinic acid production is the reductive pathway in the cytoplasm of a recombinant yeast. Alternative pathways, including strategies that employ enzymes of the mitocondrially-compartmentalized tricarboxylic acid cycle, can also function for the production of organic acids such as malic acid and succinic acid. In order to attain improved systems for producing organic acids such as malic acid and succinic acid, consideration must be given to the compartmentalization of both the organic acids as well as biosynthetic enzymes, transport proteins, and other factors (e.g. ATP, co-factors) required for organic acid production.

In some embodiments, the recombinant yeast is functionally transformed to increase the activity of a PYC polypeptide or a PPC polypeptide, together with modifications to increase the activities of a MDH polypeptide and a MAE polypeptide. The yeast can be as described above.

After a modified (e.g., recombinant) yeast has been obtained, the yeast can be cultured in a medium. The medium in which the yeast can be cultured can be any medium known in the art to be suitable for this purpose. Culturing techniques and media are well known in the art. In one embodiment, culturing can be performed by aqueous fermentation in an appropriate vessel. Examples for a typical vessel for yeast fermentation comprise a shake flask or a bioreactor.

The medium can comprise a carbon source such as glucose, sucrose, fructose, lactose, galactose, or hydrolysates of vegetable matter, among others. In some embodiments, the medium can also comprise a nitrogen source as either an organic or an inorganic molecule. Alternatively or additionally, the medium can comprise components such as amino acids; purines; pyrimidines; corn steep liquor; yeast extract; protein hydrolysates; water-soluble vitamins, such as B complex vitamins; and inorganic salts such as chlorides, hydrochlorides, phosphates, or sulfates of Ca, Mg, Na, K, Fe, Ni, Co, Cu, Mn, Mo, or Zn, among others. Further components known to one of ordinary skill in the art to be useful in yeast culturing or fermentation can also be included. The medium can be buffered but need not be.

The carbon dioxide source can be gaseous carbon dioxide (which can be introduced to a headspace over the medium or sparged through the medium) or a carbonate salt (for example, calcium carbonate) incorporated into the media.

During the course of the fermentation, the carbon source is internalized by the yeast and converted, through a number of steps, into malic acid. Expression of the MAE polypeptide allows the malic acid so produced to be secreted by the yeast into the medium. Typically; some amount of the carbon source is converted into succinic acid and some amount of the succinic acid is secreted by the yeast into the medium.

An exemplary media include: mineral medium containing 50 g/L CaCO₃ and 1 g/L urea and or mineral medium containing 1 g/L urea and sparged with air complemented with 20% CO₂.

According to the present disclosure, modified yeast can be cultured under conditions and for a time sufficient for malic and/or succinic acid to accumulate to a predetermined amount. For example, the malic and/or succinic acid may accumulate to about 0.3 moles of malic and/or succinic acid/moles of substrate, about 0.35 moles of malic and/or succinic acid/moles of substrate, about 0.4 moles of malic and/or succinic acid/moles of substrate, about 0.45 moles of malic and/or succinic acid/moles of substrate, 0.5 moles of malic and/or succinic acid/moles of substrate, about 0.55 moles of malic and/or succinic acid/moles of substrate, about 0.6 moles of malic and/or succinic acid/moles of substrate, about 0.65 moles of malic and/or succinic acid/moles of substrate, about 0.7 moles of malic and/or succinic acid/moles of substrate, about 0.75 moles of malic and/or succinic acid/moles of substrate, about 0.8 moles of malic and/or succinic acid/moles of substrate, about 0.85 moles of malic and/or succinic acid/moles of substrate, about 0.9 moles of malic and/or succinic acid/moles of substrate, about 0.95 moles of malic and/or succinic acid/moles of substrate, about 1 moles of malic and/or succinic acid/moles of substrate, about 1.05 moles of malic and/or succinic acid/moles of substrate, about 1.1 moles of malic and/or succinic acid/moles of substrate, about 1.15 moles of malic and/or succinic acid/moles of substrate, about 1.2 moles of malic and/or succinic acid/moles of substrate, about 1.25 moles of malic and/or succinic acid/moles of substrate, about 1.3 moles of malic and/or succinic acid/moles of substrate, about 1.35 moles of malic and/or succinic acid/moles of substrate, about 1.4 moles of malic and/or succinic acid/moles of substrate, about 1.45 moles of malic and/or succinic acid/moles of substrate, about 1.5 moles of malic and/or succinic acid/moles of substrate, about 1.55 moles of malic and/or succinic acid/moles of substrate, about 1.6 moles of malic and/or succinic acid/moles of substrate, about 1.65 moles of malic and/or succinic acid/moles of substrate, about 1.7 moles of malic and/or succinic acid/moles of substrate, about 1.75 moles of malic and/or succinic acid/moles of substrate. In some embodiments, the malic or succinic acid accumulates in the medium. In some embodiments the substrate is glucose.

We have observed that culturing a recombinant yeast of the present disclosure in mineral medium comprising 50 g/L CaCO₃ and 1 g/L urea can lead to levels of malic acid (as acid) in the medium of at least 1 g/L. In one embodiment, it can lead to levels of malic acid (as acid) in the medium of at least 10 g/L In a fluffier embodiment, it can lead to levels of malic acid (as acid) in the medium of at least 30 g/L.

Thus in certain embodiments the malic and/or succinic acid accumulates in the medium to at least about 20 g/L, at least about 30 g/L, at least about 40 g/L, at least about 50 g/L, at least about 60 g/L, at least about 70 g/L, at least about 80 g/L, at least about 90 g/L, at least about 100 g/L, at least about 110 g/L, at least about 120 g/L, at least about 130 g/L, at least about 140 g/L, at least about 150 WL, at least about 160 g/L, at least about 170 g/L, at least about. 180 g/L, at least about 190 g/L, at least about 200 g/L.

According to the present disclosure, modified yeast can be cultured under conditions where the acidic pH of the medium promotes the accumulation of soluble free malic and/or succinic acid as the major product form, thereby decreasing economic and environmental costs that result from the need to remove impurities or by-products such as calcium sulfate (gypsum). Thus in certain embodiments the malic and/or succinic acid accumulates in a medium of a pH of at least less than 5.0, at least less than 4.5, at least less than 4.0, at least less than 3.5, at least less than 3.0, at least less than 2.5.

After culturing has progressed for a sufficient length of time to produce a desired concentration of malic acid or succinic acid (e.g., in the medium), the malic acid or succinic acid can be isolated. Specifically, the organic acid (e.g. the malic acid or succinic acid) can be brought to a state of greater purity by separation of the organic acid from at least one other component (either another organic acid or a compound not in that category) of the yeast or the medium. In some embodiments, the organic acid is at least about 50%, 55%, 60%, 65%, 70%, 75%, 80%, 85%, 90%, 95%, 96%, 97%, 98%, 99%, pure or more. In some embodiments, the isolated organic acid is at least about 95% pure, such as at least about 99% pure.

Any available technique can be utilized to isolate accumulated malic and/or succinic acid accumulated. For example, the isolation can comprise purifying the organic acid (e.g. malic acid and/or succinic acid) from the medium by known techniques, such as the use of an ion exchange resin, activated carbon, microfiltration, ultrafiltration, nanofiltration, liquid-liquid extraction, crystallization, or chromatography, among others. Liquid-liquid extraction is a preferred method for recovering protonated carboxylic acids such as malic and/or succinic acid from an aqueous medium. Liquid-liquid extraction is generally performed using a reactive long-chain aliphatic tertiary amine (e.g. triisooctylamine or tridodecylamine) in a extractant containing a modifier (e.g. n-octanol), which enhances the extracting power of the reactive amine, and an inert diluent (e.g. n-heptane).

The malic acid and succinic acid produced by the modified organisms described herein can be incorporated into one or more food, cosmetic and/or chemical products, for example, as described below.

Malic Acid

Malic acid is used in the production of a variety of foods. Beneficial traits of malic acid for the food industry include flavor enhancement relative to other products, desirable properties for blending with other ingredients, and chelating abilities to increase the solubility and availability of ions such as calcium. Malic acid is currently used in the production of a wide range of foods, including beverages, confectioneries (particularly sour-tasing candies) and bakery products, as well as food preservatives.

In beverages, malic acid improves flavors and masks the tastes of some salts and sweeteners; it also improves pH stability and provides several desirable properties to calcium fortified drinks. In candies, malic acid provides lingering sourness and exceptional blending properties, including its high solubility at relatively low temperatures. Malic acid functions to provide consistent texture and balanced flavor in bakery products. In food applications, malic acid can also be used in edible and antimicrobial films and coatings, which can also be further treated with a variety of powdered ingredients.

Malic acid is also currently utilized in the cosmetic industry, for example as part of face and/or body lotions, as well as in nail enamel compositions that are made of polymers plasticized with esters of malic acid.

Malic acid is also utilized in the chemical industry; and has significant potential for many high-volume applications derived from a malic acid feedstock. These applications include, for example, surfactants, industrial chemicals such as maleic anhydride, 1,4-butanediol, tetrahydrofuran, hydroxybutyrolactone and hydroxysuccinate, and biodegradable polymers (e.g. polymalic acid and other polymers derived at least partially from malic acid monomers).

Succinic Acid

Succinic acid is currently marketed as a surfactant/detergent/extender/foaming agent. Succinic acid is also useful as an ion chelator. For instance, succinic acid is commonly utilized in electroplating in order to reduce corrosion or pitting of metals.

Succinic acid is also utilized in the food industry, for example, as an acidulant/pH modifier, a flavoring agent (e.g., in the form of sodium succinate), and/or an anti-microbial agent. Succinic acid can also be employed as a feed additive. Succinic acid can be utilized to improve the properties of soy proteins in food or feed through the succinylation of lysine residues.

Succinic acid also finds utility in the pharmaceutical/health products market, for example in the production of pharmaceuticals (including antibiotics), amino acids, vitamins, etc.

Succinic acid can also be utilized as a plant growth stimulant.

Succinic acid further can be employed in the commodity and/or specialty chemicals markets, for example as an intermediate in the production of compounds such as adipic acid (e.g., for use as the precursor to nylon and/or in the manufacture of lubricants, foams, and/or food products), 4-amino butanoic acid, aspartic acid, 1,4-butanediol (e.g., for use as a solvent and/or as raw material for production of polybutylene terephthalate resins and/or automotive or electrical parts), diethyl succinate (e.g., for use as a solvent for cleaning metal surfaces or for paint stripping), ethylenediaminedisuccinate (e.g., as a replacement for EDTA), fumaric acid, gamma-butyrolactone (e.g., for use in paint removers and/or textile products, and/or as the raw material for production of pyrrolidone derivatives), hydroxysuccinimide, itaconic acid, maleic acid, maleic anhydride, maleimide, malic acid, N-methylpyrrolidone (e.g., for use as a solvent), 2-pyrrolidione, succinimide, tetrahydrofuran (e.g., for use as a solvent and/or in adhesives, printing inks, magnetic tapes, etc), or other 4-carbon compounds.

Succinic acid can also be utilized to modify other compounds and thereby to improve or adjust their properties. For example, succinylation of proteins (e.g., on lysine residues) can improve their physical or functional attributes; succinylation of cellulose can improve water absorbitivity; succinylation of starch can enhance its utility as a thickening agent, etc.

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

In general, any modification may be applied to a cell to increase or impart production and/or accumulation of malate or a compound that can be produced in the cell using malate. In many cases, the modification comprises a genetic modification. In general, genetic modifications may be introduced into cells by any available means including chemical mutation and/or transfer (e.g., via transformation or mating) of nucleic acids. A nucleic acid to be introduced into a cell according to the present invention may be prepared by any available means. For example, it may be extracted from an organism's nucleic acids or synthesized by chemical means. Nucleic acids to be introduced into a cell may be, but need not be, in the context of a vector.

### EXAMPLE 1

### Construction of Useful Yeast Strains

Two yeast strains were constructed starting with S. cerevisiae strain TAM (MATa pdc1(-6,-2)::loxP pdc5(-6,-2)::loxP pdc6(-6,-2)::loxP ura3-52 (PDC-reduced)), which was transformed with genes encoding a pyruvate carboxylase (PYC), a malate dehydrogenase (MDH), and an organic acid transport (MAE) polypeptide.

Because the TAM strain has only one. auxotrophic marker, we disrupted the TRP1 locus in order to be able to introduced more than one plasmid with an auxotrophic marker, resulting in RWB961 (MATa pdc1(-6,-2)::loxP pdc5(-6,-2)::loxP pdc6(-6,-2)::loxP mutx ura3-52 trp1::Kanlox).

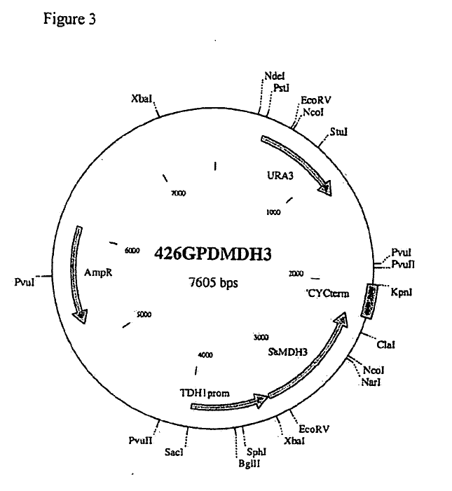

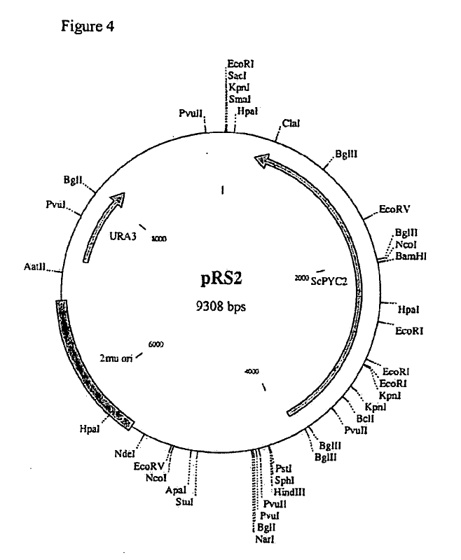

The MDH and PYC genes we used had been previously cloned into plasmids p426GPDMDH3 (2μ plasmid with URA3 marker, containing the MDH3ΔSKL gene between the S. cerevisiae TDH3 promoter and the S. cerevisiae CYC1 terminator, FIG. 3) and pRS2 (2μ plasmid with URA3 marker containing the S. cerevisiae PYC2 gene, FIG. 4).

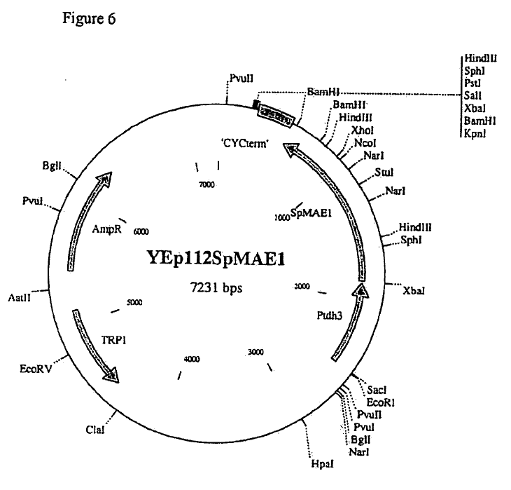

A P_{TDH3}-SpMAE1 cassette carrying the S. pombe MAE was recloned into YEplac112 (2μ, TRP1) and YIplac204 (integration, TRP1), resulting in YEplac112SpMAE1 (FIG. 6) and Ylplac204SpMAE1 (not shown).

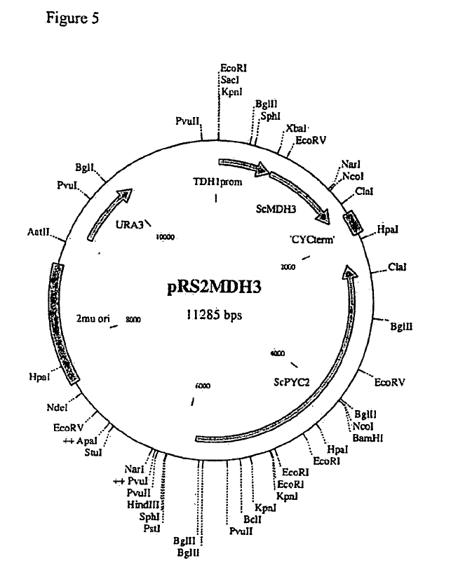

A PYC and MDH vector was prepared: pRS2MDH3ΔSKL (2μ, URA3, PYC2, MDH3ΔSKL) (FIG. 5).

RWB961 was transformed with pRS2MDH3ΔSKL and YEplac112SpMAE1 (strain 1) or pRS2MDH3ΔSKL and YIplac204SpMAE1 (strain 2). Both strain 1 and strain 2 overexpressed PYC2 and MDH3ΔSKL, but had different levels of expression for the SpMAE1, estimated at about 10-40 copies per cell for YEplac112SpMAE1 (2μ-based) and about 1-2 copies per cell for YIplac204SpMAE1 (integrated).

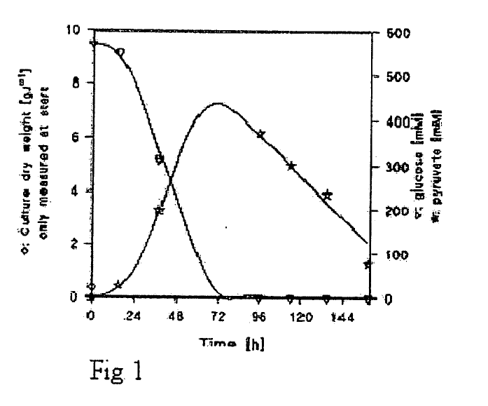

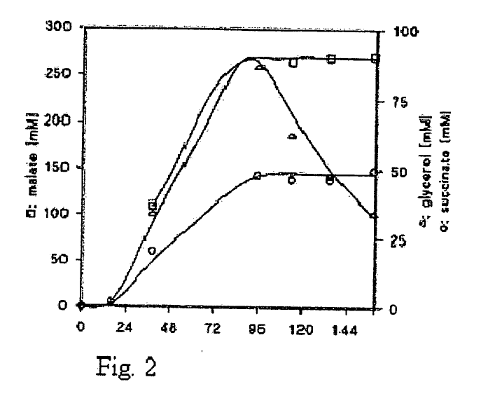

After isolation of strain 1 and strain 2, 0.04 g/L or 0.4 g/L of each strain was introduced to a 500 mL shake flask containing 100 mL mineral medium, 50 g/L CaCO₃, and 1 g/L urea. Flasks were shaken at 200 rpm for the duration of each experiment. Samples of each culture medium were isolated at various times and the concentrations of glucose, pyruvate, glycerol, succinate, and malate determined. Extracellular malate concentrations of about 250 mM after about 90-160 hr were observed. Results are shown in FIGS. 1-2.

The results indicate that the following modifications to yeast metabolic pathways allow high levels of extracellular malate accumulation by recombinant yeasts:

1. Direct the pyruvate flux towards pyruvate carboxylase (by reducing PDC activity)

2. Increase flux through pyruvate carboxylase by overexpressing PYC.

3. Introduce high malate dehydrogenase activities in the cytosol to capture oxaloacetate formed by PYC.

4. Introduce a heterologous organic acid transport polypeptide (e.g. malic acid transporter) to facilitate export of malate.

FIG. 2 also shows that extracellular succinate concentrations of about 50 mM could be produced simultaneously with the malate production described above.

### EXAMPLE 2

### Effect of Carbon Dioxide on Malate Production

The effect of carbon dioxide on malate production in a fermenter system was studied using a TAM strain overexpressing Pyc2, cytosolic Mdh3, and a S. pombe Mae1 transporter (YEplac112SpMAE1), as described in Example 1. Three fermenter experiments were performed:

A: Batch cultivations under fully aerobic conditions.

B: Batch cultivations under fully aerobic conditions with a mixture of N₂/O₂/CO₂ of 70%/20%/10%.

C: Batch cultivations under fully aerobic conditions with a mixture of N₂/O₂/CO₂ of 65%/20%/15%.

Protocol

Media

The mineral medium contained 100 g glucose, 3 g KH₂PO₄, 0.5 g MgSO₂.7H₂O and 1 ml trace, element solution according to Verduyn et al (Yeast 8: 501-517, 1992) per liter of demineralized water. After heat sterilization of the medium 20 min at 110° C., 1 ml filter sterilized vitamins according to Verduyn et al (Yeast 8: 501-517, 1992) and a solution containing 1 g urea were added per liter. Addition of 0.2 ml per liter antifoam (BDH) was also performed. No CaCO₃ was added.

Fermenter Cultivations

The fermenter cultivations were carried out in bioreactors with a working volume of 1 liter (Applikon Dependable Instruments, Schiedam, The Netherlands). The pH was automatically controlled at pH 5.0 by titration with 2 M potassium hydroxide. The temperature, maintained at 30° C., is measured with a Pt100-sensor and controlled by means of circulating water through a heating finger. The stirrer speed, using two rushton impellers, was kept constant at 800 rpm. For aerobic conditions, an air flow of 0.5 l.min⁻¹ was maintained, using a Brooks 5876 mass-flow controller (Brooks BV, Veenendaal, The Netherlands), to keep the dissolved-oxygen concentration above 60% of air saturation at atmospheric pressure.

In batches B and C, increased carbon dioxide concentration of 10% or 15% while maintaining a good oxygenation was reached by mixing pressurized air 79% N₂+21% O₂ and a gas mixture containing 79% CO₂ +21% O₂ (Hoekloos, Schiedam, the Netherlands). The desired percentage of 10% or 15% CO₂, supplied via a Brooks mass-flow controller, was topped up with pressurized air to a fixed total flow rate of 0.5 L/min.

The pH, DOT and KOH/H₂SO₄ feeds were monitored continuously using an on-line data acquisition & control system (MFCS/Win, Sartorius BBI Systems).

Off-Gas Analysis

The exhaust gas of the fermenter cultivations was cooled in a condenser (2° C.) and dried with a Perma Pure dryer (type PD-625-12P). Oxygen and carbon dioxide concentrations were determined with a Rosemount NGA 2000 gas analyser. The exhaust gas flow rate was measured with a Saga Digital Flow meter (Ion Science, Cambridge). Specific rates of carbon dioxide production and oxygen consumption were calculated as described by van Urk et al (1988, Yeast 8: 501-517).

Sample Preparation

Samples for biomass, substrate and product analysis were collected on ice. Samples of the fermentation broth and cell free samples (prepared by centrifugation at 10.000×g for 10 minutes) were stored at −20° C. for later analysis.

Determinations of Metabolites

HPLC-Determinations

Determination of sugars, organic acids and polyols were determined simultaneously using a Waters HPLC 2690 system equipped with an HPX-87H Aminex ion exclusion column (300×7.8 mm, BioRad) (60° C., 0.6 ml/min 5 mM H2SO4) coupled to a Waters 2487 UV detector and a Waters 2410 refractive index detector.

Enzymatic Metabolite Determinations

In order to verify the HPLC measurements and/or exclude separation errors, L-malic acid was determined with an enzymatic kit (Boehringer-Mannheim, Catalog No. 0 139 068).

Determination of Dry Weight

The dry weight of yeast in the cultures was determined by filtering 5 ml of a culture on a 0.45 μm filter (Gelman Sciences). When necessary, the sample was diluted to a final concentration between 5 and 10 gl⁻¹. The filters were kept in an 80° C. incubator for at least 24 hours prior to use in order to determine their dry weight before use. The yeast cells in the sample were retained on the filter and washed with 10 ml of demineralized water. The filter with the cells was then dried in a microwave oven (Amana Raderrange, 1500 Watt) for 20 minutes at 50% capacity. The dried filter with the cells was weighed after cooling for 2 minutes. The dry weight was calculated by subtracting the weight of the filter from the weight of the filter with cells.

Determination of Optical Density (OD₆₆₀)

The optical density of the yeast cultures was determined at 660 nm with a spectrophotometer; Novaspec II (Amersham Pharmasia Biotech, Buckinghamshire, UK) in 4 ml cuvets. When necessary the samples were diluted to yield an optical density between 0.1 and 0.3.

Batch A: fully aerated 21% O₂ (+79% N₂)

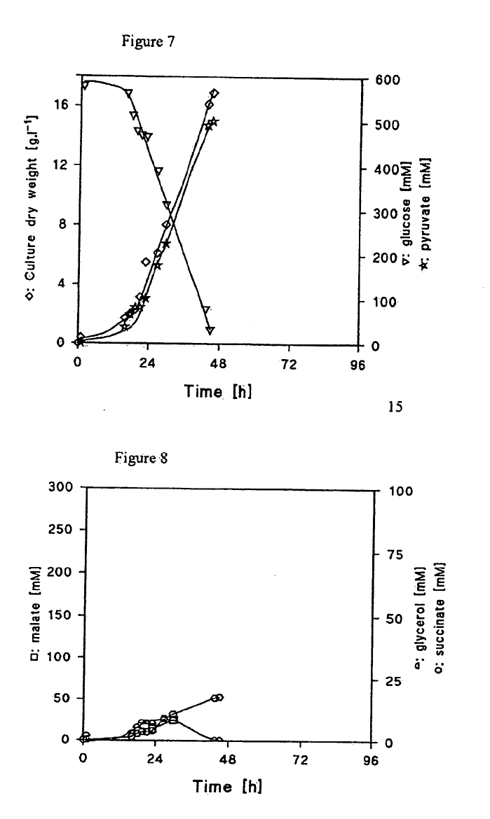

FIGS. 7 and 8 show metabolite formation against time. The result of one representative batch experiment per strain is shown. Replicate experiments yielded essentially the same results. FIG. 7 denotes the biomass (rectangle), the consumption of glucose (triangle) and the production of pyruvate (star). FIG. 8 denotes production of malate (square), glycerol (upper semi circle), and succinate (octagon). As shown in FIG. 8, the yeast produced about 25 mM malate after 24 hr and about 20 mM succinate after 48 hr.

Batch B: 10% CO₂+21% O₂(69% N₂)

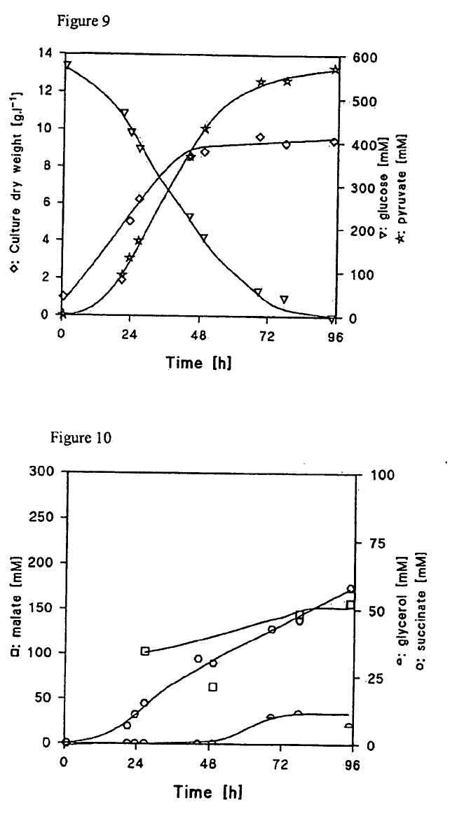

FIGS. 9 and 10 show metabolite formation against time. FIG. 9 denotes the biomass (rectangle), the consumption of glucose (triangle) and the production of pyruvate (star). FIG. 10 denotes production of malate (square), glycerol (upper semi circle), and succinate (octagon). As shown in FIG. 10, the yeast produced about 100 mM malate after 24 hr and about 150 mM malate after 96 hr, as well as about 60 mM succinate after 96 hr.

Batch C: 15% CO₂+21% O₂ (+64% N₂)

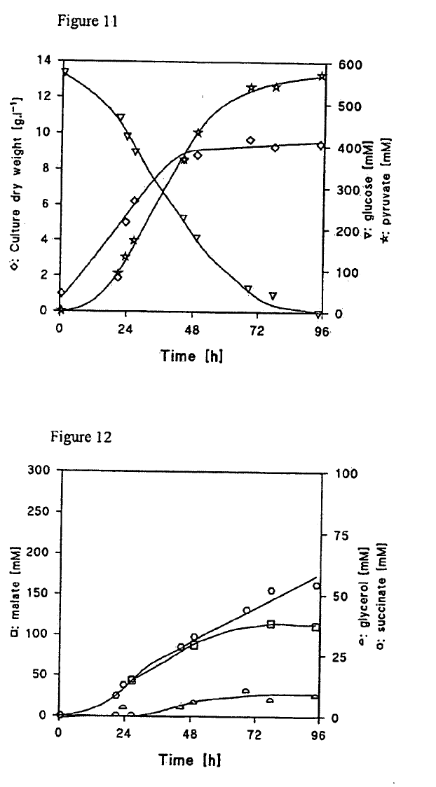

FIGS. 11 and 12 show metabolite formation against time. FIG. 11 denotes the biomass (rectangle), the consumption of glucose (triangle) and the production of pyruvate (star). FIG. 12 denotes production of malate (square), glycerol (upper semi circle), and succinate (octagon). As shown in FIG. 10, the yeast produced about 45 mM malate after 24 hr and about 100 mM malate after 96 hr, as well as about 60 mM succinate after 96 hr.

### EXAMPLE 3

### Preparation Cell-Free Extracts for Enzyme Determinations

The enzyme samples were obtained from cells growing in chemostat or from shake flasks. When the sample was obtained from shake flask for cells that did not grow on glucose these were first pre-grown on mineral medium with ethanol after which they were transferred to mineral medium with glucose. For preparation of cell extracts, 62.5 mg of biomass were harvested by centrifugation (5 min at 5000 rpm), washed once and re-suspended in 5 ml freeze buffer (10 mM potassium phosphate buffer (pH 7.5) containing 2 mM EDTA). These samples were stored at −20° C. Before preparation of cell extracts, samples were thawed, washed once and re-suspended in 4 ml sonication buffer (100 mM potassium-phosphate buffer (pH 7.5) containing 2 mM MgCl₂ and 1 mM dithiothreitol). Prior to sonication, a teaspoon of glass beads (425-600 μm diameter) was added. Extracts were prepared by sonication in a Sanyo Soniprep 150-sonicator using a 7-8 μm peak-to-peak amplitude for 4 min at 0.5 min intervals. Unbroken cells and debris were removed by centrifugation at 4° C. (20 min at 36,000×g). The clear supernatant was used as the cell extract.

### EXAMPLE 4

### Enzyme Assays

All enzyme activities were coupled to (dis)appearance of NAD(P)H or acetylCoA (acetylCoA measured via DTNB (5,5-dithiobis-(2-nitrobenzoic acid)), which was monitored spectrophotometrically at 340 nm (ε=6.3 l.mM⁻¹.cm⁻¹) or 412 nm (ε=13.6 l.mM⁻¹.cm⁻¹) respectively. Specific activity of the enzymes was calculated after protein determination via the Lowry method. All enzymes are expressed as Units ((mg) protein) ⁻¹. One unit equals 1 μmol of substrate converted per minute under the reaction conditions of the assay. Concentrations are given as the final concentration of each component in the reaction mixture (1 ml in a glass cuvette). In all cases, the reaction rates were checked to be linearly proportional to the amount of cell extract added to the assay.

PPC—E. coli. PPC—pyruvate carboxylase (4.1.1.32):

Imidazole-HCl (pH 6.6) 100 mM, NaHCO₃ 50 mM, MgCl₂ 2 mM, Glutathione 2 mM, ADP 2.5 mM, NADH 2.5 mM, MDH 3 U. Start reaction with: Phosphoenolpyruvate (2.5 mM).

PPC-E. coli PPC—pyruvate carboxylase (4.1.1.32):

### (Alternative Assay Based on Acetyl-CoA)

Tris-HCl (pH 7.5) 100 M, MgSO₄ 10 mM, KHCO₃ 10 mM, AcCoA 20 mM, KHCO₃ 10 mM, DTNB (5,5-dithiobis-(2-nitrobenzoic acid))/Tris 0.1 mM, citrate synthetase. Start reaction with PEP (5 mM).

### EXAMPLE 5

### Wild-Type and Mutant E. coli PPC Sensitivity to Malate

Wild-type and E. coli PPC mutants were analyzed for inhibition in the presence of malate. Overproduction of E. coli PPC was achieved using the pAN10ppc plasmid (Flores, C. L., Gancedo, C (1997) Expression of PPC from Escherichia coli complements the phenotypic effects of pyruvate carboxylase mutations in Saccharomyces cerevisiae. FEBS Lett. 412: 531-534), containing E. coli ppc gene behind the ADM promoter. Two amino acid changes, K620S and K773G, of E. coli PPC have been reported to affect the inhibition of E. coli PPC by aspartate and malate (Kai et al (2003) Phosphoenolpyruvate carboxylase: three-dimensional structure and molecular mechanisms. Arch Biochem Biophys. Jun 15;414(2):170-9). Oligonucleotide-based site-directed mutagenesis was performed to generate ppc alleles that encoded putative malate-insensitive ppc polypeptides. Two oligonucleotides were designed in order to introduce both these mutations in plasmid pAN10ppc. Both mutant plasmids, pAN10ppcmut5 and pAN10ppcmut10 were introduced into wild-type S. cerevisiae CEN.PK113-5D.

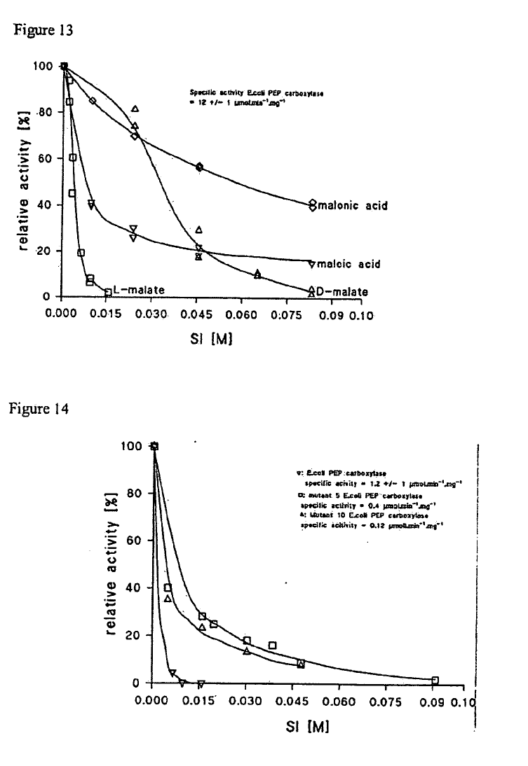

Cell extracts from glucose-grown shake-flask cultures were tested to determine the inhibition of malate as described in Examples 3 and 4 herein. The specific activity of wild-type E. coli PPC is inhibited in the presence of malate (FIG. 13). In contrast, the specific activities of the pAN10ppcmut5 and pAN10ppcmut10 versus the wild-type E. coli PPC were 0.4, 0.24 and 1.2 μmol.min⁻¹.mg.protein⁻¹ respectively. In the presence of 0.01 M malate, the wild-type E. coli PPC was fully inhibited while both mutants, K620S (mutant 5) and K773G (mutant 10), still retained 40% of their initial activity (FIG. 14).

### EXAMPLE 6

### Regulatory Sequences

Sequences which consist of, consist essentially of, and comprise the following regulatory sequences (e.g. promoters and terminator sequences, including functional fragments thereof) may be useful to control expression of endogenous and heterologous genes in engineered host cells, and particularly in engineered fungal cells described herein.

TDH3 Promoter

5′cagtttatcattatcaatactcgccatttcaaagaatacgtaaataat
taatagtagtgattttcctaactttatttagtcaaaaaattagcctttta
attctgctgtaacccgtacatgcccaaaatagggggcgggttacacagaa
tatataacatcgtaggtgtctgggtgaacagtttattcctggcatccact
aaatataatggagcccgctattaagctggcatccagaaaaaaaaagaatc
ccagcaccaaaatattgttttcttcaccaaccatcagttcataggtccat
tctcttagcgcaactacagagaacaggggcacaaacaggcaaaaaacggg
cacaacctcaatggagtgatgcaacctgcctggagtaaatgatgacacaa
ggcaattgacccacgcatgtatctatctcattttcttacaccttctatta
ccttctgctctctctgatttggaaaaagctgaaaaaaaaggttgaaacca
gttccctgaaattattcccctacttgactaataagtatataaagacggta
ggtattgattgtaattctgtaaatctatttcttaaacttcttaaattcta
cttttatagttagtcttttttttagttttaaaacaccagaacttagtttc
gacggatt 3′

ADH1 Promoter

5′cgccgggatcgaagaaatgatggtaaatgaaataggaaatcaaggagc
atgaaggcaaaagacaaatataagggtcgaacgaaaaataaagtgaaaag
tgttgatatgatgtatttggctttgcggcgccgaaaaaacgagtttacgc
aattgcacaatcatgctgactctgtggcggacccgcgctcttgccggccc
ggcgataacgctgggcgtgaggctgtgcccggcggagttttttgcgcctg
cattttccaaggtttaccctgcgctaaggggcgagattggagaagcaata
agaatgccggttggggttgcgatgatgacgaccacgacaactggtgtcat
tatttaagttgccgaaagaacctgagtgcatttgcaacatgagtatacta
gaagaatgagccaagacttgcgagacgcgagtttgccggtggtgcgaaca
atagagcgaccatgaccttgaaggtgagacgcgcataaccgctagagtac
tttgaagaggaaacagcaatagggttgctaccagtataaatagacaggta
catacaacactggaaatggttgtctgtttgagtacgctttcaattcattt
gggtgtgcactttattatgttacaatatggaagggaactttacacttctc
ctatgcacatatattaattaaagtccaatgctagtagagaaggggggtaa
cacccctccgcgctcttttccgatttttttctaaaccgtggaatatttcg
gatatccttttgttgtttccgggtgtacaatatggacttcctcttttctg
gcaaccaaacccatacatcgggattcctataataccttcgttggtctccc
taacatgtaggtggcggaggggagatatacaatagaacagataccagaca
agacataatgggctaaacaagactacaccaattacactgcctcattgatg
gtggtacataacgaactaatactgtagccctagacttgatagccatcatc
atatcgaagtttcactaccctttttccatttgccatctattgaagtaata
ataggcgcatgcaacttcttttcttatttttcttttctctctcccccgtt
gttgtctcaccatatccgcaatgacaaaaaaatgatggaagacactaaag
gaaaaaattaacgacaaagacagcaccaacagatgtcgttgttccagagc
tgatgaggggtatctcgaagcacacgaaactttttccttccttcattcac
gcacactactctctaatgagcaacggtatacggccttccttccagttact
tgaatttgaaataaaaaaaagtttgctgtcttgctatcaagtataaatag
acctgcaattattaatcttttgtttcctcgtcattgttctcgttcctttc
ttccttgtttattttctgcacaatatttcaagctataccaagcatacaat
caactccaagctggccgct 3′

TEF1 Promoter

5′tagcttcaaaatgtttctactccttttttactcttccagattttctcg
gactccgcgcatcgccgtaccacttcaaaacacccaagcacagcatacta
aatttcccctctttcttcctctagggtgtcgttaattacccgtactaaag
gtttggaaaagaaaaaagagaccgcctcgtttattttcttcgtcgaaaaa
ggcaataaaaatttttatcacgtttctttttcttgaaaatattttttttg
atttttttctctttcgatgacctcccattgatatttaagttaataaacgg
tcttcaatttctcaagtttcagtttcatttttcttgttctattacaactt
tttttacttcttgctcattagaaagaaagcatagcaatctaatctaagtt
tt3′

### EXAMPLE 7

### Preparation of Samples for Intracellular Metabolite Measurements

Biomass samples (4 ml of a 4 g dry weight/l suspension) were taken from an anaerobic fermentation assay and immediately quenched with 20 ml 60% methanol at −40° C. After washing the cells twice with cold 60% methanol, intracellular metabolites were extracted by resuspending the cell pellets in 5 ml of boiling 75% ethanol and incubating them for 3 min at 80° C. Cell debris and intracellular metabolites were dried at room temperature with a vacuum evaporator (Savant Automatic Environmental SpeedVac® System type AES 1010). Finally, 0.5 ml of demineralized water was added. The resulting suspension was stored at −20° C. Before metabolite analysis, the suspension was centrifuged.

### EXAMPLE 8

### Overexpression of Modified MDH Isoenzymes

MDH containing plasmids were constructed similar to those described in McAlister-Henn et al. (1995). Expression and function of a mislocalized form of peroxisomal malate dehydrogenase (Mdh3) in yeast. J Biol Chem. 1995 September 8;270(36):21220-5 and Small W C, McAlister-Henn L (1997) Metabolic effects of altering redundant targeting signals for yeast mitochondrial malate dehydrogenase. Arch Biochem Biophys. August 1;344(1):53-60. The first was a MDH1 gene from which the first 17 amino acids were removed, Mdh1ΔL. Deletion of the first 17 amino acids was expected to allow partial cytosolic relocation with much of Mdh1ΔL still localizing to its normal compartment, the mitochondria. The second construct was the MDH3 gene from which the 3′ SKL sequence was removed, Mdh3ΔSKL. Mdh3ΔSKL was expected to localize to the cytosol instead of the peroxisome. Both MDH constructs were expressed from the TDH3 promotor.

The total in vitro Mdh activity measured in strains with these constructs was over 4 to 20 fold that of a wild-type S. cerevisiae strain (CEN.PK113-13D). In shake flask fermentations on glucose, the enzyme activity varied between 30 to 90 μmol.min⁻¹.mg.protein⁻¹ (Table A).

Table A: Average Mdh activities in wild-type and S. cerevisiae strains expressing the plasmid containing Mdh1ΔL and Mdh3ΔSKL

| Strains | total-MDH activity | [μmol · min⁻¹ · mg⁻¹] |
|---|---|---|
| CEN.PK113-13D | 5 | |
| MDH1ΔL-construct^{#1} | 20→90 | |
| MDH3ΔSKL-construct^{#1} | 20→44 | |

^{#1}Enzyme samples from mineral medium (M.M.) with glucose 2% culture after pre-culturing on M.M. ethanol 2%

The sub-cellular fractionation of a wild-type strain, expressing Mdh3ΔSKL from the TDH3 promoter, grown in a nitrogen-limited continuous culture, showed that over 60% of the total Mdh activity was cytosolic. The rest of the activity was associated with the membrane fraction, which includes the mitochondria and peroxisomes.

Additional proof for the localization of the mutated Mdh enzymes was obtained by complementing mdh mutants. A S. cerevisiae strain with a deletion of the MDH1 gene, coding for mitochondrial Mdh, cannot grow with acetate as the carbon source, while deletion of the MDH2 gene, coding for the cytosolic Mdh, results in an inability to grow on mineral media with acetate or ethanol as the carbon source. Transformation of an mdh2 strain with plasmids containing MDH1ΔL and MDH34ΔSKL showed that both could complement the phenotype and therefore both are active in the cytosol. Transformation of an mdh1 mutant with the same constructs showed that MDH3ΔSKL could not complement the mdh1 phenotype (no growth on acetate) while MDHL1ΔL could. Therefore Mdh3ΔSKL is only active in the cytosol, not in the mitochondria, while Mdh1ΔL is active in both compartments.

Cultivation of S. cerevisiae CEN.PK113-32D (wt) with p425GPDMDH3ΔSKL (2μ plasmid, LEU2, TDH3 promotor, overproduction MDH3 without terminal SKL sequence) was performed in continuous culture. The cultivations were performed at a growth rate of 0.1 h⁻¹ under nitrogen-limited conditions at pH 5. Metabolite measurements were performed as described in Example 7 herein. The malate production did not exceed those found in wild-type S. cerevisiae strains without the MDH3ΔSKL construct, namely 0.03 g.1⁻¹. The carbon recovery was 97% yielding a stoichiometric balance of: C₆H₁₂O₆ (glucose)+0.034 NH₃ (ammonia)+0.8 O₂→0.71 CH_{1.8}O_{0.5}N_{0.2} (biomass)+2.2 CO₂+0.02 C₃H₈O₃ (glycerol)+0.01 C₄H₆O4 (succinate)+0.002 C₄H₆O₅ (malate)+0.09 C₃H₄O₃ (pyruvate)+1.4 C₂H₆O+1.6 H₂O.

Therefore, although in vitro studies showed Mdh1ΔL and Mdh3ΔSKL increase malate dehydrogenase activity, cultivation of the strains yielded carbon dioxide and biomass as main products and no significant malate production was shown. Further genetic and/or other manipulations (e.g. further comprising organic acid transport polypeptides) in the context of MDH1ΔL and MDH3ΔSKL comprising strains may yield strains with increased observable malate production.

### EXAMPLE 9

### pdc Strain Construction and Malic Acid Production Analysis

TABLE 1
CENPK182 was crossed to CEN.P

| | |
|---|---|
| CEN.PK182 | MATa pdc1::loxP pdc5::loxP pdc6::loxP |
| CEN.PK2-1D | MATα ura3-52 trp1-289 leu2-3,112 his3Δl |
| RWB837 | MATa ura3-52 pdc1::loxP pdc5::loxP pdc6::loxP |
| TAM | MATa pdc1::loxP pdc5::loxP pdc6::loxP ura3-52 |
| | [URA3 plasmid] *evolved for Glu⁺ |
| | (able to grow in the presence of glucose) Et^{ind} |
| | (Ethanol independent-able to grow in the absence of ethanol) |

K2-1D and MATα pdc1 pdc5 ura3 trp1 (MY2219, MY2223, and MY2243 [also his3],) and MATα pdc1 pdc5 pdc6 ura3 trp1 (MY2222, MY2242 [his3], and MY2246 [his3]) progeny were identified.

RWB837 was transformed with an episomal 2 micron URA3 plasmid (YEpLpLDH) bearing the lactate dehydrogenase gene from Lactobacillus plantarum to create RWB876. RWB876 was subjected to 26 transfers through lactic acid fermentation medium (70 g/L glucose; 5 g/L ethanol) to create m850. Forty-five passages of m850 through the same medium lacking ethanol led to the isolation of Lp4f.

m850 and Lp4f were cured of their YEpLpDH plasmid, rendered trp1 ΔhisG using a hisG-URA3-hisG cassette (i.e. excision of the URA3 marker was accomplished on minimal drop-out plates containing 5-fluoroorotic acid by recombination between the hisG repeats, resulting in the clean deletion of the TRP1 gene) and serially transformed with pRS2MDH3ΔSKL and YEplac112SpMAE1 to produce MY2271 and MY2308, respectively. CEN.PK182 was likewise rendered trp1 ΔhisG, and along with MY2219, transformed with the same pair of plasmids to create MY2277 and MY2279, respectively.

MY2308 was crossed to MY2223 and MY2243 and prototrophic Glu⁺ progeny were identified, including MY2518 and MY2524.

TAM was cured of an episomal URA3 plasmid, rendered trp1 ΔhisG using a hisG-URA3-hisG cassette, and serially transformed with pRS2MDH3ΔSKL and YEplac112SpMAE1 to produce MY2264. MY2223, MY2243, MY2222, MY2242, and MY2246 were mated with MY2264 to create the diploid strains MY2300, MY2301, MY2299, MY2294, and MY2302, respectively.

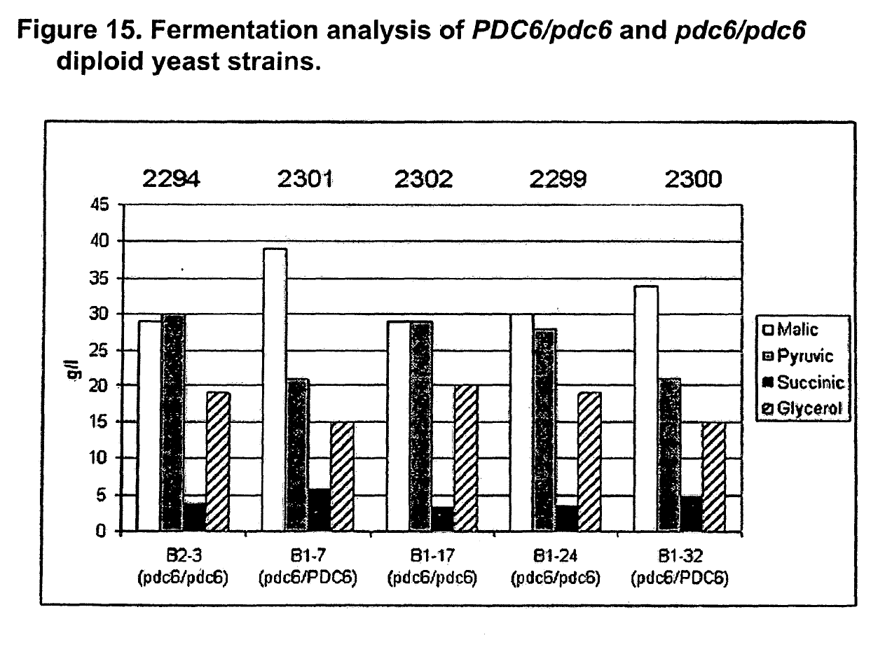

FIG. 15 shows fermentation results for these five diploids. It can be observed that the two PDC6/pdc6 strains produced higher malate to pyruvate ratios than that seen with the three pdc6/pdc6 strains. Ethanol levels were below detection.

MY2300 was sporulated and plated on minimal ammonia media supplemented with casamino acids (2 g/L), glycerol (10 g/L), and glucose (10 g/L), and prototrophic MATα segregants, including MY2433, were identified, and their pdc6 genotype determined by PCR analysis.

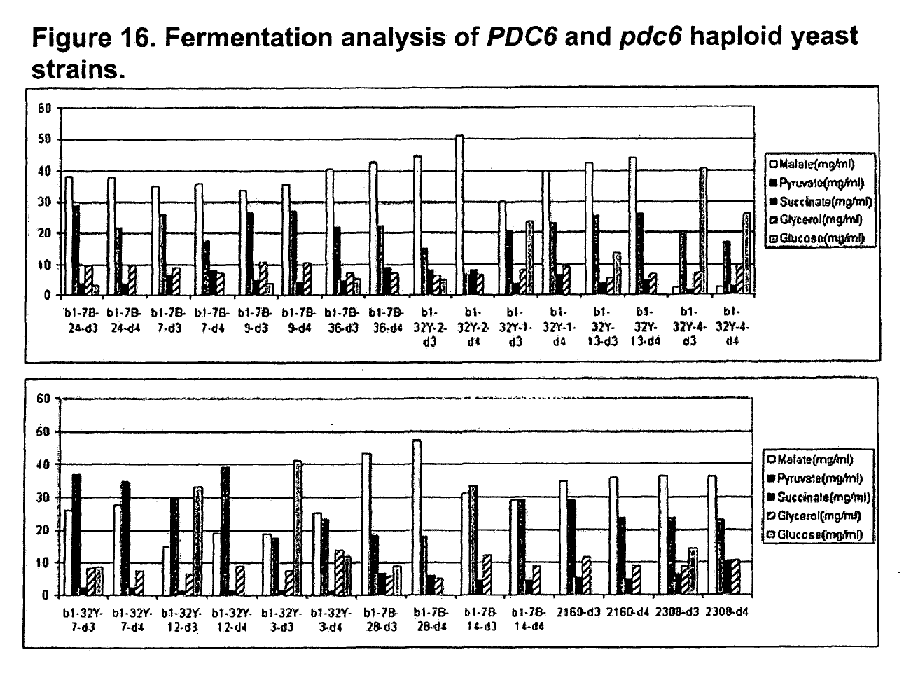

FIG. 16 shows fermentation results for 13 progeny from this cross. It can be seen that on average pdc6 progeny produced a lower ratio of malate to pyruvate than did the PDC6⁺ progeny.

RWB961, MY2264, MY2271, MY2277, MY2279, MY2308, MY2433, MY2518, and MY2524 were compared in multiple fermentations. It was found that MY2433 and MY2518 were capable of producing in excess of 50 g/L malic acid (from 100 g/L glucose), and that MY2308 could produce up to 35 g/L. MY2271, MY2277, and MY2279 produced malic acid at a level not quite approaching that seen with the TAM derivatives RWB961 and MY2264 (20-30 g/L).

### EXAMPLE 10

### Malate Dehydrogenase Variant

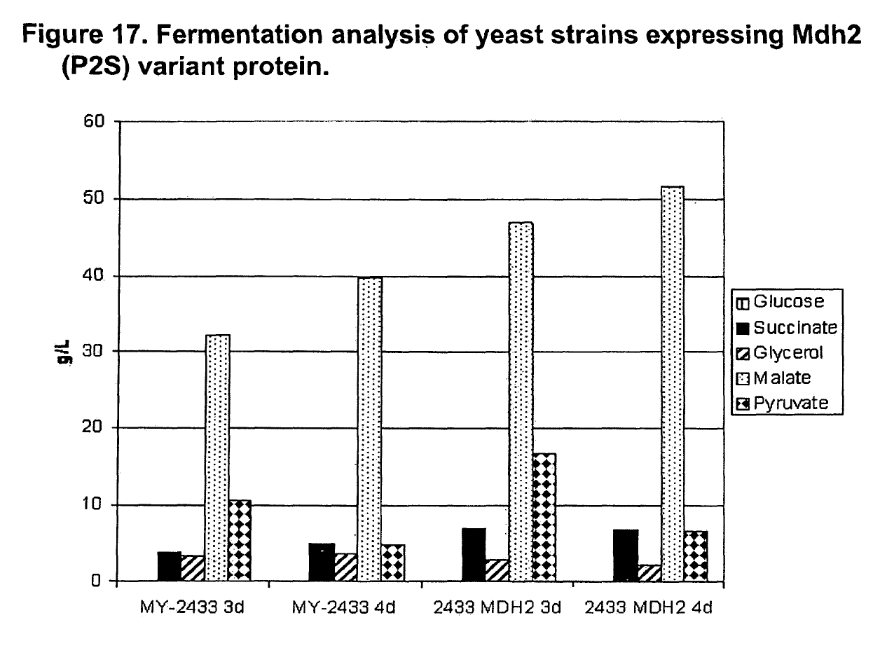

In order to create a variant of Mdh2 (MDH2-P2S) not subject to catabolite inactivation, we engineered a mutation in the coding sequence that encodes a serine rather than a proline at the second position after the start codon. MO5448 (5′-CACACACTAGTAGTAACATGTCTCACTCAGTTACACCATCC-3′) and M05449 (5′-CACACCTCGAGTTAAGATGATGCAGATCTCGATGCA-3′) were used to amplify a 1.0 kb fragment from S. cerevisiae genomic DNA by PCR, which was subsequently cleaved with XhoI and SpeI, and ligated to MluI-XbaI-cleaved pRS2MDH3ΔSKL along with the 178 by MluI-XhoI CYClt fragment from pRS413TEF, to create pMB4978. When strains carrying pMB4978 were compared with isogenic strains carrying pRS2MDH3ΔSKL in shake flask fermentations, a consistent improvement was seen. For example, when was cured of pRS2MDH3ΔSKL and transformed with pMB4978, the resulting strain produced >25% more malic acid in a four-day fermentation (FIG. 17); other experiments and strain backgrounds gave similar results.

### EXAMPLE 11

### Sequence of PYC2-ext

In order to create a variant of pRS2MDH3ΔSKL in which the encoded Pyc2 protein (PYC2-ext) possesses the five amino acid carboxy terminal extension that is found in other common wild type yeast strain backgrounds, we engineered a frameshift mutation in PYC2 by inserting an additional cytosine residue into a consecutive series of four cytosine residues found near the 3′ end of the coding strand ( . . . ATCCCCAAAAA . . . ). MO5265 (5′-CACACCGTCTCAGGGGATGGGGGTAGGGTTTC-3′) and MO5183 (GCCAAGGATAATGGTGTTGA) were used to amplify a 1.3 kb fragment from pRS2MDH3ΔSKL DNA that was subsequently cleaved with EagI and BsmBI. MO5266 (5′-CACCGTCTCACCCCAAAAAAAAAGTAATTTTTACTCGTT-3′ and MO5186 (5′-GCAGCAATTAGTTGGCGACA-3′) were used to amplify a 300 by fragment from pRS2MDH3ΔSKL that was subsequently cleaved with BsmBI and MluI. These fragments were ligated to the large fragment of EagI- and MluI-cleaved pRS2MDH3ΔSKL to create pMB4968. The PYC2-ext allele in pMB4968 encodes a protein with the carboxy terminal sequence . . . EETLPPSPKKVIFTR*, instead of the sequence . . . EETLPPSQKK* encoded by the PYC2 gene of pRS2MDH3ΔSKL. When strains carrying pMB4968 were compared with isogenic strains carrying pRS2MDH3ΔSKL in shake flask fermentations, slightly higher amounts of malic acid were detected with pMB4968 (PYC2-ext). Other factors such as increasing biotinylation capacity or supplemental CO₂ could increase the utility of this allele.

### EXAMPLE 12

### Organic Acid Transporters

Genes encoding putative aluminum-activated organic acid transporters (Oatₘₐₗ proteins corresponding to those encoded by Brassica napus and Triticum secale were constructed by de novo gene synthesis as follows. The following two sequences were synthesized.

ttctagaaacaaaatggaaaaattgcgtgaaatagttagagagggaagaa
gagttggcgaagaggatcccagaagaattgtacactcatttaaagttgga
gtcgcgttggttttagttagctcattttactactatcaaccatttggtcc
atttactgactactttggtataaatgcgatgtgggccgtaatgaccgtcg
ttgttgtttttgaattttctgtcggagctactttaagtaaaggattaaat
agaggtgtcgcaactttagtcgcaggaggcctagcgttaggagcacatca
attggcttcattatcaggaaggactatagaacccattctattggctactt
ttgtatttgttacagcagcacttgctacctttgttcgttttttccccgag
agttaaggctacatttgattatggaatgctaattttcattctaactttta
gcttaatttccttatcccagtttagagacgaagaaatattagacttagct
gaatcgagattatcaactgtattagttggcggggttagttgtattttaat
ttccatatttgtttgtccagtttgggccggtcaggacttacattcactat
tagtttcaaaccttgatactctaagccactttttacaagaattcggtgat
gaatatttcgaagcgagaacatatggtaatattaaagttgttgaaaagag
aagaagaaaccttgagagatacaaatcagtgctaaactcaaaatccgatg
aagattccctagcaaatttcgcaaaatgggaaccaccacatggcaaattc
ggttttagacatccatggaaacaatatttagtcgtcgcagctttagttag
acagtgcgctcatagaatagatgctttaaactcttatattaattcaaatt
ttcaaatcccaatcgatataaaaaagaaattggaagaaccattcaggaga
atgtcattagaatctggaaaagcaatgaaagaagcttcaattagtctgaa
aaaaatgaccaaatccagcagttacgatatccatataattaatagccaat
ctgcatgcaaagccttatctaccttgttaaaatctggtatattaaacgac
gttgagccattacaaatggtgagtttactaactacagtttctttattaaa
tgacatagttaacataacagaaaaaataagtgaatctgtgagagaattgg
cttccgctgctagattcaggaataaaatgaaacctactgaaccaagtgtt
tccctaaaaaagttagattcaggttctacaggatgtgcaatgccaataaa
ttcaagggatggtgatcatgttgtaaccatattacttagtgacgatgata
aagatgatatagatgatgacgatacttcaaatatagtactagacgatgac
actattaatgaaaagtctgaagatggtgaaatacatgtacaaaccagttg
tgtaagagaggtgggaatgatgcctgaacattcacttggtgtaagaatat
tgcaaatttaactcgag-(B. napus (B.n.)).
ttctagaaacaaaatggatattgatcatggaagagaaatagatggagaaa
tggtttctactattgcgtcatgcggcttgttattgcattccttattagca
ggtttcgcaagaaaggtcggtggtgctgccagagaagatcccagaagagt
tgctcattcattaaaagtggtctagcattggctctagtttcagctgttta
ctttgtaacaccattattcaacgggttaggcgttagtgcaatttgggctg
ttcttaccgtagtcgtcgttatggagtttaccgtcggtgcaactttaagt
aaaggtttaaatagagctttggcaactttagtcgcaggatgtattgctgt
cggagcccatcaattagcagaattaacagaacgttgttcagatcaagggg
aaccagttatgttgacagtattagttttttttgtcgcatcagcagcaaca
tttcttagattcattcccgaaatcaaagcaaaatatgactatggcgtaac
tatttttatactaactttcggtttagttgctgtttcgtcttacagagtgg
aagaacttattcaattagctcatcaaagattttacacaattgtcgtcgga
gtatttatatgtctatgcacaacggtatttttatttcctgtttgggccgg
agaggacgtccataaattagcttcatcaaatttagggaaattagcgcaat
ttattgaaggtatggaaacaaactgttttggcgaaaacaacatagctatc
aatttagaaggaaaagattttttacaagtatacaaatcggttctgaattc
aaaggccactgaagattctttatgcacttttgcaagatgggaaccaagac
atggtcagtttagatttagacacccctggtctcaatatcaaaaattaggt
acactgtgtagacaatgcgcatcatcaatggaagctttagctagttacgt
tattaccaccacaaagactcaataccccgcagctgcaaatccggaacttt
cttttaaagtcagaaaaacatgtcacgaaatgtctactcatagtgctaaa
gttttaagaggtttagaaatggcaatacgtacaatgacagtcccatactt
agccaacaatacagtcgtagttgcaatgaaggccgccgagagattaagat
cagaattagaagataacgctgcacttttacaggtaatgcatatggctgtt
actgctacgttacttgccgatttagtcgatagagtcaaagaaatcacaga
atgtgttgatgttttagcaagattagcccattttaaaaatcctgaagatg
caaaatacgcaatcgttggtgctttaactagaggaatagatgatcctttg
cctgatgtagttatattataactcgag-3′ (T. secale (T.s)).

The sequences above were cleaved with XbaI and XhoI, and ligated to pRS416TDH3, pRS416TEF1, and pRS416ADH1 to produce pMB4943 (TDH3-B.n.), pMB4944 (TEF1-B.n.), pMB4945 (ADH1-B.n.), pMB4946 (TDH3-T.s.), pMB4947 (TEF1-Ts.), and pMB4948 (ADH1-Ts.); all are URA3-marked plasmids. In addition, analogous constructs were made in a TRP1-marked series of plasmids: pMB4950 (TDH3-B.n.), pMB4952 (TEF1-B.n.), pMB4954 (ADH1-B.n.), pMB4949 (TDH3-T.s.), pMB4951 (TEF1-T.s.), and pMB4953 (ADH1-T.s.).

Although no evidence for malic transport was observed when compared with the isogenic controls MY2308 (Lp4f [pRS2MDH3ΔSKL][YEplac112SpMAE1]) and MY2306 (Lp4f [pRS2MDH3ΔSKL][pRS424]) when tested in shake flask fermentations in the Lp4f background, further analysis including addition of alumininum cations, alleviation of possible cellular mislocalization, and altered growth conditions or strain backgrounds can be tested.

### EXAMPLE 13

### Expression of Heterologous Pyc Polypeptides

Genes encoding Pyc from Aspergillus niger, Yarrowia lipolylica, and Nocardioides sp. are synthesized as follows:

actagtaaatatgtctaatgttccagaaactaaagtagatccttcattgt
ccacaccagaggtccctagtcaaggtttacatagcagattggacaagatg
agagctgattcatccatattgggaagtatgaacaaaatattagtggcaaa
tagaggtgaaatcccaattagaatctttagaaccgcccacgagttatcta
tgcagactgttgctatctatgcacatgaggacagattgtcaatgcacaga
ttcaaggccgatgaggcttacgtaattggagacagaggaaaatatacacc
tgtccaagcatacttacaggtggacgagataatcgaaattgccaaggctc
atggtgttaacatggtacacccaggatatggtttcttgtccgaaaatagt
gagttcgcaagaaaagtcgaagaagctggaatggcctggattggtcctcc
acataacgttatagacagtgtcggtgacaaggtttcagcaagaaacttag
ctatcaagaacaatgtacctgtcgtgccaggaaccgatggtcctgttgag
gacccaaaggatgccttgaaatttgtagaaaagtacggttatcctgtcat
tataaaagcagctttcggaggtggaggtagaggtatgagagttgtgagag
agggagatgacatcgttgatgcctttaacagagcatccagtgaagctaag
actgccttcggtaatggtacatgtttcattgaaagattcttagacaaacc
aaaacatatagaggtacaattgttagcagatggacaaggtaatgtcgtgc
acttgtttgaaagagattgctctgttcagaggagacatcaaaaggtagtc
gaaatcgctccagccaaagacttacctgtcgaggtgagagatgcaatttt
ggacgatgctgttagattagctgaagatgccaagtacagaaacgcaggaa
ccgctgagttcttggtagacgagcaaaatagacactacttcattgagata
aacccaagaatccaggtcgaacatactattacagaggaaataaccggtat
cgatattgttgccgcacaaatacagattgctgccggtgcaactttagagc
aattgggattaacacaagacaaaatctcaactagaggttagctattcagt
gtagaataaccacagaagatcctgcaaagcaattccaaccagatactgga
aaaatcgaagtctacagatctgctggaggtaatggagtaagattggacgg
tggtaacggatttgccggtgcaattatatcccctcactatgatagtatgt
tagtcaagtgctcatgttctggcaccacattcgagatagccagaagaaag
atgattagagccttggttgagtttagaataagaggagtcaagactaatat
tccattcttattggcattattgacacatcctacctttatcgaaggaaaat
gaggactacattcattgacgatactccatccttatttgacttgatgacca
gtcagaacagggctcaaaagttattggcctacttagcagatttatgtgtt
aatggaacaagtataaaaggtcaggtaggtaaccctaagttaaagtctga
ggtcgttatcccagtgttgaagaactccgaaggaaagattgtagattgta
gtaaacctgacccagtcggttggagaaatatattagttgaacaaggtcct
gaggctttcgccaaggcagtgagaaagaacgatggagttttggtaatgga
cactacctggagagatgctcatcaatcattattggctacaagagtcagaa
ctaccgacttattggcaattgcaaatgaaacatctcacgctatgtccggt
gcctttgcattagagtgctggggaggtgctacttttgacgttgcaatgag
attcttgtatgaagatccatgggacagattaagaaagatgagaaaagcag
tgccaaatatcccttttcagatgttgttaagaggtgctaatggagtagcc
tactcatctttgccagataacgcaatagatcatttcgtcaagcaagctaa
agacaatggtgttgatatctttagagtgttcgacgccttaaacgatttgg
atcaattaaaggtaggtgttgacgcagtcaagaaagctggaggtgttgtg
gaagcaaccgtatgttatagtggagatatgttgaatcctaagaagaagta
taacttagagtattacttggactttgtcgatagagttgtagaaatgggca
cccacatcttaggtattaaagatatggcaggaactttgaagccagctgcc
gcaaccaaattaataggtgctatcagagaaaagtatcctaatttgccaat
tcatgttcatacacacgactccgccggtactggagtggcatcaatggctg
ccgcagctgaggccggtgcagatgtcgttgacgtggcttctaatagtatg
tctggaatgacctcccagccttcaataagtgccttaatggcaatattgga
aggaaaattatctactggtttggacccagctttagtaagagaattggatg
cctattgggcacaaatgagattattgtactcatgcttcgaggctgactta
aagggacctgatccagaagtctttcaacatgaaattcctggtggtcagtt
gacaaacttattgttccaagcccagcaagttggattaggtgagcaatgga
aagaaactaagcaggcatatatcgctgccaatcaattgttaggagacatt
gtaaaagttaccccaacatctaaggtggtcggtgatttggcacagtttat
ggtttccaacaaattaagttacgacgatgtgataaaacaggctggttcat
tggattttcctggatctgtattagacttctttgagggtttgatgggtcaa
ccatatggaggtttcccagaacctttaagaactgaagcattaagaggaca
gagaaagaaattaaccgagaggcctggaaaatccttgcctccagtcgatt
ttgcagctgttagaaaagacttagaagaaagattcggtcacatcacagag
tgtgatattgccagttactgcatgtatcctaaggtatttgaagattacag
aaagatagttgacaagtatggagatttgtcaattgtgccaactagattat
tcttggaagcacctaaaaccgacgaggaattttctgtcgaaatcgagcaa
ggtaagacattaatattggctttaagagctattggtgatttgtccatgca
aactggattaagagaagtttacttcgagttgaatggtgaaatgagaaaga
tcagtgtggaagataagaaagccgcagtagaaaccgtgtcaagaccaaaa
gccgaccctggaaacccaaatgaagttggtgcccctatggccggtgtagt
tgtggaagtcagagttcatgagggaacagaagtgaagaaaggtgatccag
tagctgtcttatctgccatgaagatggaaatggttatttccgccccagtc
tcaggtaaagtaggagaggtcccagttaaggaaggtgactctgttgatgg
aagtgatttgatatgcaaaatcgtgagagcttaactcgagctagcgaaga
caaccag (Y. lipolytica Pyc)
actagtaaatatggctgcaccaagacaacctgaagaggccgttgatgaca
ctgagttcattgatgaccatcacgatcagcatagagacagcgtacacacc
agattgagagctaattcagcaataatgcaattccagaaaatcttagtcgc
caacagaggtgagattccaataagaatctttagaaccgctcatgaattgt
ccttacaaactgtggcagtttatagtcacgaagatcatttgtctatgcat
agacaaaaggccgatgaggcttacatgattggaaagagaggtcagtatac
acctgtaggagcatacttagctatagacgaaatcgtcaagattgccttgg
aacacggtgtgcacttaattcacccaggttatggattcttgtcagagaat
gcagaatttgctagaaaagttgaacaatccggtatggtattcgtcggacc
taccccacaaactatagagagtttaggtgataaggtttctgccagacagt
tggcaatcagatgtgacgtgcctgttgtaccaggtacacctggaccagtc
gaaagatacgaggaagtgaaggcttttaccgatacttatggtttccctat
tataatcaaggccgcatttggtggaggtggaagaggtatgagagttgtaa
gagatcaagctgaattaagagactcattcgagagagccacatccgaagca
agaagtgcttttggtaacggaaccgtgttcgttgaaagattcttggatag
accaaaacatattgaggtgcagttattgggtgacaatcacggtaacgtgg
tacacttatttgaaagagattgtagtgtgcaaaggagacatcaaaaggtg
gttgaaatagcccctgcaaaagatttgccagctgacgtaagagatagaat
cttagctgacgccgtcaagttggcaaaatcagttaattacagaaacgctg
gaactgccgagttcttagtggatcagcaaaatagatattacttcattgaa
attaacccaagaatacaagttgaacacaccatcactgaggaaattaccgg
tatagatatcgtagcagctcagattcaaatagccgcaggagctacattgg
agcagttaggtttgactcaagacagaatttccaccagaggtttcgcaatc
caatgtagaattacaactgaagatcctagtaagggattttctccagacac
aggaaaaatagaagtctatagatcagctggtggaaatggtgttagattag
atggaggtaatggtttcgccggagcaatcattacccctcattacgattct
atgttggtgaaatgcacttgtagaggttccacatatgagatcgccagaag
aaaggtagtcagagccttagttgagtttagaatcagaggtgtgaaaacta
acattccattcttgacctccttattgtcacaccctgtgtttgtggatgga
acatgctggactaccttcatagatgacacaccagaattatttgcattggt
cggttctcagaatagggctcaaaagttattggcctacttaggagatgttg
cagtgaacggttccagtattaaaggtcaaatcggagagcctaagttgaaa
ggtgacattataaagccagtattacatgatgctgccggtaaacctttgga
tgtctcagttccagcaactaagggatggaaacagatcttagactctgaag
gtcctgaggcttttgctagagccgtgagagcaaataagggatgtttgatt
atggataccacatggagggacgctcatcaatccttattggccactagagt
tagaaccatagacttattgaacattgcacacgagacaagtcatgctttag
ccaatgcatattcattggaatgttggggtggtgctactttcgatgtagca
atgagattcttatacgaggacccatgggatagattgagaaaattaagaaa
agcagtccctaatatcccattccaaatgttgttaagaggagctaatggtg
ttgcctattcttccttgccagacaacgcaatataccacttttgcaagcag
gctaagaagtgtggtgtggatattttcagagtatttgatgccttaaacga
cgtcgatcaattggaagttggaatataagcagtgcatgctgccgaaggtg
tagttgaggcaacaatttgctattcaggagatatgttaaacccttctaag
aaatacaacttgccatactacttagatttggtcgataaggttgtgcagtt
caaacctcacgtattaggtataaaggatatggctggtgtcttgaaaccac
aagccgcaagattattgatcggaagtattagagaaagataccctgacttg
cctatacatgttcatacacacgactccgctggtactggtgtagcttcaat
gattgcatgtgctcaagccggagcagatgctgttgatgccgcaaccgact
ctttgagtggtatgacatctcagcctagtatcggagctatcttagcctca
ttggaaggtactgagcatgatccaggtttaaacagtgcacaagtgagagc
tttggacacatattgggcccaattaagattgttatactctccttttgaag
caggattgactggtccagatcctgaagtctatgagcacgaaataccaggt
ggacagttaaccaacttgatcttccaggcttcacagttaggtttgggaca
acaatgggccgaaacaaagaaagcatacgagtctgctaatgacttattgg
gtgacgttgtgaaagtaactcctacctccaaggtcgttggtgacttagcc
cagtttatggtaagtaacaaattgacagcagaggacgttattgctagagc
cggagagttagattttccaggttcagtgttggagttcttagaaggtttga
tgggacaaccatatggtggatttcctgagccattaagaagtagagcattg
agagacagaagaaagttagataaaagacctggtttgtacttagaaccatt
ggacttagctaagatcaaatcccaaattagagaaaattatggtgctgcca
ctgagtacgacgtcgcaagttatgctatgtaccctaaggttttcgaagat
tataagaagtttgtggccaaattcggagacttgtcagtattaccaaccag
atacttcttggcaaagcctgaaatcggtgaggagttccatgtcgaattag
agaaaggtaaggttttgatattaaagttgttagctattggaccattgtct
gaacagacaggtcaaagagaggtgttttatgaagttaacggagaagtgag
acaggtgtccgttgatgataagaaggccagtgtggagaatactgcaagac
ctaaagctgaattaggtgactcatctcaggtgggagccccaatgtccgga
gtcgttgtagaaatcagagttcatgatggtttggaggtgaagaaaggtga
ccctattgcagtcttatcagctatgaagatggaaatggttatatctgcac
ctcacagtggaaaagtgtcctcattgttagtaaaggaaggtgattctgtc
gatggacaagacttggtttgcaaaatcgtgaaggcttaactcgagctagc
gaagacaaccag-3′ (A. niger Pyc)
actagtaaatatgttttccaaagttttggtagctaatagaggtgagattg
ccataagagccttcagagctgcatatgaattaggagccagaactgtcgct
gtctttccatacgaagatagatggtcagagcatagattgaaagccgacga
ggcttacgagatcggagaaagaggacaccctgttagagcttacttggacc
cagaagcaattgtagcagtcgccataagagccggtgccgatgcagtgtat
cctggttacggtttcttgtccgaaaacccagcattggccgaggcctgtgc
aaacgctggtatcacatttgtaggtcctaccgccgatgtattgactttaa
caggtaacaaagcaagagcaattgctgcagctaccgctgccggtgtccct
actttagcaagtgttgaaccttctadtgacgtggacgccttggtggaatc
agccggagagttgccatacccattattcgtaaaggcagtggctggtggag
gtggtagaggaatgagaagagttgatgcaccaggtcaattgagagaagca
gttgagacatgtatgagagaagctgaaggtgcatttggcgaccctactgt
attcatagagcaggctgtcgttgatccaagacatatcgaagtgcaagtat
tggcagadggtgaaggtcacgtaatgcatttgtttgagagagattgttcc
gtccagaggagacaccagaaagtgattgaaatcgcccctgctccaaactt
agacccagagttgagagacagaatatgcgcagacgccgttagattcgcta
aggaaatcggatacagaaatgccggtactgtcgagttcttattggacgca
aaaggaacctatcatttcattgaaatgaatcctagaatacaagtcgagca
tacagtgactgaagaggtgacagatgtagacttagtacagagtcaattga
gaatcgcttctggtgaaaccttagccgacttgggattatcacaagaaact
gtaaccttgagaggagctgcattgcagtgtagaattactacagaggaccc
agctaacaactttagacctgacactggtgttatcacaacttacagatccc
caggaggtggaggagtgagattggatggtggtactgtgtatactggtgcc
gaagtcagtgcccactttgattctatgttagctaagttgacttgcagagg
tagaaccttcgagaaagccgttgagaaggcaagaagagctgtggccgagt
ttagaatcagaggtgtttcaacaaacattcctttcttgcaagccgtattg
gtggacccagacttttccagtggacatgttactacctctttcattgaaac
acacccacaattattgcaagccagatcatctggtgacagaggaagcagat
tgttgcattacttagccgatgtgactgtgaatcaaccacacggtcctgca
cctgtttccatcgaccctgttaccaaattgccagaggtgaacttagacgt
tcctgctccagatggtacaagacagttgttgttagatgttggaccagaag
agtttgccagaagattaagagcacaaactggtgttgctgtaaccgataca
actttcagggacgcccatcaatcattgttagctaccagagtgagaacaag
agatttgttagctgtagccggtcatgtcgcaagaactacccctcagttgt
ggtattagaggcttggggaggtgccacatatgatgtagccttaagattct
tagctgaggacccatgggagagattggcagccttaagacaagcagtgcct
aacatctgtttgcagatgttattgagaggaagaaatactgtaggttacac
accttatccagccgatgttactcaagcattcgtcgaagaagctgccgcaa
ccggtattgacgtgtttagaatatttgatgctttaaacgatgtggagcaa
atgaggccagccatagaggctgtaagagctacaggaactgccgtcgcaga
agttgcattgtgttacacaggagacttatccgatcctgacgagacattgt
atactttagattactatttggaattagccgatagaattgtagacgccgga
gcacacgtcttagctataaaggatatggcaggattattgagagtgccagc
tgccagaaccttagtcacagcattgagagacagattcgacttgccagttc
atttgcacactcatgataccccaggtggacagttagctacattattggca
gccattgacgccggtgtggatgctgtagacgccgcaactgctagtatggc
aggaacaacatcacaacctccattgtctgcattagtttccgctactgatc
atggacctagagaaaccggtttgagtttaggtgccgtgtcagcattggag
ccatattgggaagctacaagaagagtatacgcacctttcgagtctggatt
accttccccaactggtagagtttatagacacgaaatccctggaggtcaat
tgtcaaacttaagacagcaagctatcgccttaggtttgggagagaaattc
gagcaaatagaagatatgtacgcagctgccaacgacatattaggtaatgt
ggtcaaggttacccctttctagtaaggtagtaggtgacttagcattgcac
ttagtcgctgttggagccgaccctacagaatttgcagatgagccaggaaa
attcgatattcctgactccgtaataggattcttaaatggagaattgggtg
acccacctggaggttggccagaacctttcagaactaaggccttagctggt
agaactcacaagcctcctgttgaggaattagacgatgaacagagagaggg
attggccggttcatctccaacaagaagaagaactttaaacgaattgttat
ttccaggtccaacaaaggagttcacagaaagtagattaagattttggtga
cacttctgtgttaccaacattggattacttatatggttgagaagaggaga
agagcatgcagtcgaaatcgaagagggtaaaacattaatcttgggagttc
aagccataactgaacctgatgaaagaggattcagaaccgtgatgacaact
attaacggtcagttaagaccagtgagtgtcagagacagatcagttgccgc
tgaggttgctgccgcagaaaaggcagataccagtaaacctggacacgttg
cagccccatttcaaggtgtggtgtctatcgttgtggaggaaggtcaacag
gtagccgctggagacacagtagcaattatcgaagccatgaagatggaggc
ctcaataaccgcacctgttgccggaacagttgagagattggccttatctg
gtactcaagcagtagaaggaggtgatttggtcttagttttgtcctaactc
gagctagcgaagacaaccag-3′ (Nocardioides sp. Pyc)

Plasmids harboring the Pyc-encoding genes are constructed by treating the synthetic DNA with SpeI and XhoI and ligating to XbaI-XhoI-cleaved pRS426TEF, pRS426ADH1, or pRS426TDH3. These plasmids can be introduced into strains in which overexpressed Mdh-encoding constructs have been integrated at the can1 locus, and which also express OAT_{Mal}.

### EXAMPLE 14

### Expression of Heterologous Phosphoenolpyruvate Carboxylase (Ppc) Polypeptides

The gene encoding Ppc is amplified from Erwinia chrysanthemi DNA with primers MO3764 (5′ATGAATGAACAATATTCCGCCA3′) and MO3765 (5′TTAGCCGGTATTGCGCATCC3′). The resulting 2.6 kb fragment is subsequently ligated to SmaI-cleaved pBluescriptIISK⁻ to create pMB4077. This plasmid is cleaved with PstI and BamHI, the ends made blunt with the Klenow enzyme, and ligated to the URA3 vectors (e.g. pRS416TDH3, pRS416ADH1, or pRS416TEF1) which have been treated with XbaI and XhoI followed by the Klenow enzyme. The resultant expression cassettes may be moved as SacI-XhoI blunted fragments to pRS2MDH3ΔSKL either by blunt end ligation into the unique Mlul site of pRS2MDH3ΔSKL or replacement of the PYC2 gene in pRS2MDH3ΔSKL by blunt end ligation of the cassettes into PstI- and BsiWI-cleaved pRS2MDH3ΔSKL.

The resultant plasmids may be used in place of pRS2MDH3ΔSKL, in the Pdc⁻ strains described above containing YEplac112SpMAE1, and assayed for malic production.

### EXAMPLE 15

### Expression of an Organic Acid Transporter to Increase C4 Acid Production

Production of organic acids, e.g., malic acid can be increased in a fungal cells by modifying the fungal cell to express a protein (e.g., a dicarboxylic acid transporter or exporter/importer- an organic acid transport polypeptide) that allows export of an organic acid such a as C4 organic acid. This permits export of organic acids that might otherwise suppress additional organic acid synthesis.

A sequence encoding a putative dicarboxylic acid transporter from Aspergillus oryzae (GenBank Accession No. XP_{—}001820881; DCAT) was synthesized. The sequence used, optimized for S. cerevisiae codon bias, follows.

TTCTAGAAACAAAATGTTTAATAACGAGCATCATATACCTCCTGGATCTA
GCCACTCGGATATTGAAATGTTAACTCCTCCTAAATTTGAAGATGAAAAA
CAACTTGGACCTGTCGGTATAAGAGAAAGACTTAGACACTTTACTTGGGC
TTGGTATACACTAACTATGAGTGGGGGCGGCTTAGCTGTTTTAATAATTT
CACAACCTTTTGGTTTCAGAGGTCTTAGGGAAATCGGAATCGCTGTTTAT
ATTCTAAATCTTATACTTTTTGCTTTAGTTTGTTCCACTATGGCTATTAG
GTTTATACTACATGGTAATTTATTAGAAAGTTTGCGTCATGATAGAGAAG
GTTTGTTCTTTCCCACATTCTGGCTTTCAGTTGCAACAATTATATGTGGT
TTATCAAGGTATTTCGGTGAAGAATCAAATGAAAGTTTTCAGCTAGCTTT
AGAAGCTCTGTTCTGGATTTATTGCGTTTGTACACTATTAGTAGCTATTA
TACAATATTCATTCGTTTTCTCCTCTCATAAATATGGTCTACAAACTATG
ATGCCATCTTGGATACTACCAGCTTTTCCTATAATGTTGTCAGGTACTAT
TGCGTCTGTTATTGGCGAGCAACAACCAGCTAGAGCAGCTTTACCTATAA
TCGGAGCAGGTGTAACTTTTCAAGGATTAGGTTTTTCAATTTCTTTTATG
ATGTATGCACACTATATTGGTCGTCTAATGGAATCTGGTTTACCACACTC
AGATCATAGACCTGGTATGTTTATATGTGTTGGTCCACCGGCCTTTACAG
CACTAGCCTTAGTCGGTATGTCTAAGGGTTTGCCTGAAGATTTTAAGTTA
TTACATGATGCACACGCCCTGGAAGATGGAAGAATTATAGAACTATTAGC
AATCTCTGCAGGTGTTTTCTTATGGGCTTTAAGTTTATGGTTTTTTCGTA
TTGCAATTGTCGCCGTTATCAGATCACCTCCCAAAGCCTTTCATTTAAAC
TGGTGGGCTATGGTTTTCCCAAACACTGGTTTCACTTTAGCAACAATAAC
CCTAGGTAAAGCATTAAACTCTAACGGTGTAAAAGGTGTTGGTTCAGCTA
TGAGTATTTGTATTGTATGTATGTATATATTCGTTTTCGTAAATAATGTT
AGAGCTGTGATACGTAAAGATATAATGTACCCTGGTAAAGACGAAGATGT
CTCTGATTAGTCTTCTCGAG

THE AMINO ACID SEQUENCE OF THE ENCODED ORGANIC ACID TRANSPORTER FOLLOWS.

MFNNEHHIPPGSSHSDIEMLTPPKFEDEKQLGPVGIRERLRHFTWAWY
TLTMSGGGLAVLIISQPFGFRGLREIGIAVYILNLILFALVCSTMAIR
FILHGNLLESLRHDREGLFFPTFWLSVATIICGLSRYFGEESNESFQL
ALEALFWIYCVCTLLVAIIQYSFVFSSHKYGLQTMMPSWILPAFPIML
SGTIASVIGEQQPARAALPIIGAGVTFQGLGFSISFMMYAHYIGRLME
SGLPHSDHRPGMFICVGPPAFTALALVGMSKGLPEDFKLLHDAHALED
GRIIELLAISAGVFLWALSLWFFCIAIVAVIRSPPKAFHLNWWAMVFP
NTGFTLATITLGKALNSNGVKGVGSAMSICIVCMYIFVFVNNVRAVIR
KDIMYPGICDEDVSD

The transporter-encoding nucleic fragment was liberated from its vector using XbaI and XhoI, and ligated to XbaI-XhoI-cleaved pRS416GPD to create pMB5210 (CEN URA3). The TDH3p-DCAT1-CYC1t cassette was moved to pRS404 using KpnI and Sad to create pMB5238 (integrating TRP1). Spontaneous Trp⁻ revertants were obtained from MY2888 and MY2907 as fluoro-anthranilate-resistant clones, and MY3229 (Pyc⁻¹) and MY3230 (Pyc⁺) were identified as having simultaneously lost TRP1 and TDH3-Spmae1 by homologous excision. Next, pMB5238 was used to transform MY3230 to prototrophy (via integration at the trp1 locus), creating MY3523, MY3524, and MY3525. Alternatively, pMB5238 was used to transform MY3229 to tryptophan prototrophy (via integration at one of two resident CYC1 terminators), creating MY3300, which was subsequently transformed to uracil prototrophy with pMB5165 (directed to integrate at the pyc2 locus), creating MY3522. These four Pyc⁺ Dcat⁺ strains are predicted to be virtually genetically identical, and they behave similarly in fermentations. On average the four strains were capable of producing greater than 16 g/L malic acid in 96 hr when cultured with 100 g/L glucose and 0.5% CaCO₃. In comparison, strain MY2907, containing the S. pombe mae1 transporter instead of DCAT1, typically produces 12 to 15 g/L malic acid under these poorly buffered conditions (final pH<3).

Additional useful organic acid transporter polypeptides are listed in FIGS. 24 and 26.

### EXAMPLE 16

### Production of Malic Acid in Low Ph Cultures

Fungal strains used for production of malic acid are generally culture at around pH 4.5; bacterial strains used for the production of organic acids such as malic and succinic acids often require even greater buffering (e.g. culturing at pH 7 for many strains). However, maintaining the pH near neutrality during malic acid production can incur significant economic and environmental costs. For example, base addition during fermentation can have considerable cost ramifications. Furthermore, buffered fermentations result in the production of a salt of a particular organic acid, whereas the desired product is the free acid. In most instances, the resulting culture broth (or concentrated versions thereof) must be acidified in order to recover the free acid. This acidification (through e.g. sulfuric acid) incurs further raw material costs, and this also results in the formation of considerable quantities of low value by-products such as CaSO4 (i.e. gypsum). Therefore, highly buffered processes can be economically inviable due to factors such as the costs for materials and disposal of waste products such as gysum. Thus, the ability to produce malic acid in lower pH cultures (e.g., pH 2.5) would have significant benefits with respect to the economics and environmental impact of the downstream processing and purification steps. For instance, a hundred-fold reduction in the amount of CaSO₄ would be possible if the final pH could be reduced from pH 4.5 to pH 2.5.

Reduced buffering and/or low pH culturing is difficult, during the production of malic acid because the production of pyruvate by pdc1 pdc5 strains leads, at low pH, to the generation of protonated pyruvic acid, which is toxic. To address this issue, a pyc1 pyc2 strain, MY2888, whose malic production could be increased upon introduction of a Pyc-encoding plasmid, whose flux to ethanol is reduced but not eliminated, and which secretes undetectable levels of pyruvate. Described below are strains derived from MY2888 which produce high levels of malic acid even when cultured at low pH.

TAM was cured of an episomal URA3 plasmid, rendered trp1hisG using a hisGURA3-hisG cassette, and a TDH3p-MDH3ΔSKL cassette was integrated at the can1 locus by URA3-mediated integration and excision to create MY2421. Subsequent integration of a TDH3p-Spmae1 TRP1 plasmid (pMB4957) at the same locus yielded MY2542.

A pyc1 pyc2 strain (CMJ238) of the W303 background was obtained from Carlos Gancedo (University of Madrid). After HO-mediated mating type switching to MATα to create MY2682, this strain was mated to MY2542, sporulated, and a glucose-ammonia-negative antimycin-sensitive spore was identified MY2888. Its genotype was determined to be pyc1 pyc2 PDC1 pdc5 PDC6 can1::TDH3p-MDH3ΔSKL::TRP1::TDH3p-Spmae1 MTH1ΔT. Introduction of TDH3p-PYC2 in a single copy at the pyc2 locus (pMB5165) of MY2888 results in a strain, MY2907, capable of substantial malic production (see Table XXX). Introduction of multiple episomal copies of TDH3p-YlPYC (pMB5094) into MY2888 results in a strain, MY2928, with even higher productivity (Table XXX). Moreover, the lack of pyruvate secretion allows for the malic production under poorly buffered conditions (see Table 2, Column 4).

TABLE 2
Typical malic acid productivity in shake flasks (g/L)

| | 100 g/L Glu 50 g/L | 200 g/L Glu 100 g/L | 100 g/L Glu Ca- |
|---|---|---|---|
| Strain | CaCO₃ | CaCO₃ | MES 20% CO₂ |
| MY2907 | >55 | >100 | ~20 |
| MY2928 | >65 | ~140 | ~30 |
| final pH | ~5 | ~5 | ~2.5 |

Media conditions with CaCO₃ were according to Verduyn, lacking ammonium sulfate and containing 1 g/L urea. In the third column, the same medium was used with 13 mM Ca-MES (2 mM Ca⁺⁺) pH 5.7 in a 20% CO₂ atmosphere.

Plasmid pMB5165 (TDH3p-PYC2 URA3) was prepared as follows. Oligo MO5316 (CACACACTagtaaaatatgagcagtagcaagaaattg) was used to insert a SpeI site upstream of the PYC2 open reading frame in pRS2MDH3ΔSKL by PCR amplification (pMB4972; also contains the TP11 promoter in place of native PYC2 promoter). A fragment comprising the PYC2 open reading frame and the PYC2 terminator was subsequently ligated as a 3.5 kb SpeI-BsiWI fragment to SpeI-Acc65I-cleaved pRS414GPD to create pMB5099. The TDH3p-PYC2 cassette was then moved as a BglI fragment to BglI-cleaved pRS406 to create pMB5165.

Plasmid pMB5094 (TDH3p-YlPYC URA3 2m) was prepared as follows. A nucleic acid molecule having the sequence below, encoding the Y. lipolytica pyruvate carboxylase using S. cerevisiae codon bias, was synthesized:

actagtaaatatgctaatgttccagaaactaaagtagatccttcattgtc
cacaccagaggtccctagtcaaggtttacatagcagattggacaagatga
gagctgattcatccatattgggaagtatgaacaaaatattagtggcaaat
agaggtgaaatcccaattagaatctttagaaccgcccacgagttatctat
gcagactgttgctatctatgcacatgaggacagattgtcaatgcacagat
tcaaggccgatgaggcttacgtaattggagacagaggaaaatatacacct
gtccaagcatacttacaggtggacgagataatcgaaattgccaaggctca
tggtgttaacatggtacacccaggatatggtttcttgtccgaaaatagtg
agttcgcaagaaaagtcgaagaagctggaatggcctggattggtcctcca
cataacgttatagacagtgtcggtgacaaggtttcagcaagaaacttagc
tatcaagaacaatgtacctgtcgtgccaggaaccgatggtcctgttgagg
acccaaaggatgccttgaaatttgtagaaaagtacggttatcctgtcatt
ataaaagcagctttcggaggtggaggtagaggtatgagagttgtgagaga
gggagatgacatcgttgatgcctttaacagagcatccagtgaagctaaga
ctgccttcggtaatggtacatgtttcattgaaagattcttagacaaacca
aaacatatagaggtacaattgttagcagatggacaaggtaatgtcgtgca
cttgtttgaaagagattgctctgttcagaggagacatcaaaaggtagtcg
aaatcgctccagccaaagacttacctgtcgaggtgagagatgcaattttg
gacgatgctgttagattagctgaagatgccaagtacagaaacgcaggaac
cgctgagttcttggtagacgagcaaaatagacactacttcattgagataa
acccaagaatccaggcgaacatactattacagaggaaataaccggtatcg
atattgttgccgcacaaatacagattgctgccggtgcaactttagagcaa
ttgggattaacacaagacaaaatctcaactagaggttttgctattcagtg
tagaataaccacagaagatcctgcaaagcaattccaaccagatactggaa
aaatcgaagtctacagatctgctggaggtaatggagtaagattggacggt
ggtaacggatttgccggtgcaattatatcccctcactatgatagtatgtt
agtcaagtgctcatgttctggcaccacattcgagatagccagaagaaaga
tgattagagccttggttgagtttagaataagaggagtcaagactaatatt
ccattcttattggcattattgacacatcctacctttatcgaaggaaaatg
ctggactacattcattgacgatactccatccttatttgacttgatgacca
gtcagaacagggctcaaaagttattggcctacttagcagatttatgtgtt
aatggaacaagtataaaaggtcaggtaggtaaccctaagttaaagtctga
ggtcgttatcccagtgttgaagaactccgaaggaaagattgtagattgta
gtaaacctgacccagtcggttggagaaatatattagttgaacaaggtcct
gaggctttcgccaaggcagtgagaaagaacgatggagttttggtaatgga
cactacctggagagatgctcatcaatcattattggctacaagagtcagaa
ctaccgacttattggcaattgcaaatgaaacatctcacgctatgtccggt
gcctttgcattagagtgctggggaggtgctacttttgacgttgcaatgag
attcttgtatgaagatccatgggacagattaagaaagatgagaaaagcag
tgccaaatatcccttttcagatgttgttaagaggtgctaatggagtagcc
tactcatctttgccagataacgcaatagatcatttcgtcaagcaagctaa
agacaatggtgttgatatctttagagtgttcgacgccttaaacgatttgg
atcaattaaaggtaggtgttgacgcagtcaagaaagctggaggtgttgtg
gaagcaaccgtatgttatagtggagatatgttgaatcctaagaagaagta
caacttagagtattacttggactttgtcgatagagttgtagaaatgggca
cccacatcttaggtattaaagatatggcaggaactttgaagccagctgcc
gcaaccaaattaataggtgctatcagagaaaagtatcctaatttgccaat
tcatgttcatacacacgactccgccggtactggagtggcatcaatggctg
ccgcagctgaggccggtgcagatgtcgttgacgtggcttctaatagtatg
tctggaatgacctcccagccttcaataagtgccttaatggcaacattgga
aggaaaattatctactggtttggacccagctttagtaagagaattggatg
cctattgggcacaaatgagattattgtactcatgcttcgaggctgactta
aagggacctgatccagaagtctttcaacatgaaattcctggtggtcagtt
gacaaacttattgttccaagcccagcaagttggattaggtgagcaatgga
aagaaactaagcaggcatatatcgctgccaatcaattgttaggagacatt
gtaaaagttaccccaacatctaaggtggtcggtgatttggcacagtttat
ggtttccaacaaattaagttacgacgatgtgataaaacaggctggttcat
tggattttcctggatctgtattagacttctttgagggtttgatgggtcaa
ccatatggaggtttcccagaacctttaagaactgaagcattaagaggaca
gagaaagaaattaaccgagaggcctggaaaatccttgcctccagtcgatt
ttgcagctgttagaaaagacttagaagaaagattcggtcacatcacagag
tgtgatattgccagttactgcatgtatcctaaggtatttgaagattacag
aaagatagttgacaagtatggagatttgtcaattgtgccaactagattat
tcttggaagcacctaaaaccgacgaggaattttctgtcgaaatcgagcaa
ggtaagacattaatattggctttaagagctattggtgatttgtccatgca
aactggattaagagaagtttacttcgagttgaatggtgaaatgagaaaga
tcagtgtggaagataagaaagccgcagtagaaaccgtgtcaagaccaaaa
gccgaccctggaaacccaaatgaagttggtgcccctatggccggtgtagt
tgtggaagtcagagttcatgagggaacagaagtgaagaaaggtgatccag
tagctgtcttatctgccatgaagatggaaatggttatttccgccccagtc
tcaggtaaagtaggagaggtcccagttaaggaaggtgactctgttgatgg
aagtgatttgatatgcaaaatcgtgagagcttaactcgag

This sequence was moved as a SpeI-XhoI fragment to SpeI-XhoI-cleaved pRS426GPD to create pMB5094.

Plasmid pMB4957 (TDH3p-Spmae1 TRP1) was prepared as follows. The KpnI-SacI fragment comprising TDH3p-SpmaeI from YEplac112SpMAE1 was ligated to KpnI-SacI-cleaved pRS404 to create pMB4957.

### EQUIVALENTS

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

1-262. (canceled)

263. A method of producing an organic acid, comprising culturing a genetically modified yeast under conditions that achieve organic acid production, wherein the genetically modified yeast has a genetic modification that reduces pyruvate decarboxylase (PDC) polypeptide activity compared to an otherwise identical yeast lacking the genetic modification wherein the genetic modification that reduces PDC polypeptide activity reduces the activity of no more than two of: PDC1, PDC5 and PDC6 and at least one additional genetic modification that increases organic acid production as compared with an otherwise identical yeast lacking the modification.

264. A method of producing an organic acid, comprising culturing a genetically modified yeast under conditions that achieve organic acid production, wherein the genetically modified yeast has a genetic modification that reduces pyruvate decarboxylase (PDC) polypeptide activity compared to an otherwise identical yeast lacking the genetic modification and at least one additional genetic modification that increases organic acid production as compared with an otherwise identical yeast lacking the modification, wherein the step of culturing under conditions that achieve organic acid production comprises culturing at a pH below 5.

265. The method of claim 263 wherein the genetically modified yeast has: a) PDC1 and PDC5 activity; b) PDC1 and PDC6 activity; or c) PDC6 and PDC5 activity.

266. The method of claim 263 wherein the genetic modification that reduces PDC polypeptide activity reduces the activity of only one of: PDC1, PDC5 and PDC6.

267. The method of claim 263 wherein the genetically modified yeast is of a genus selected from the group consisting of Saccharomyces, Zygosaccharomyces, Yarrowia, Kluyveromyces or Pichia spp.

268. The method of claim 267, wherein the yeast is of the species Saccharomyces cerevisiae.

269. The method of claim 263 wherein the reduced PDC polypeptide activity is conferred by: a genetic modification that deletes at least a portion of a gene encoding a PDC polypeptide, a genetic modification that alters the sequence of a gene encoding a PDC polypeptide, a genetic modification that disrupts a gene encoding a PDC polypeptide, or a genetic modification that reduces the transcription or translation of a gene or RNA encoding a PDC polypeptide.

270. The method of claim 263, wherein the at least one modification that increases organic acid production comprises a genetic modification that increases activity of at least one polypeptide selected from the group consisting of: a pyruvate carboxylase (PYC) polypeptide, a phosphoenolpyruvate carboxylase (PPC) polypeptide, a malate dehydrogenase (MDH) polypeptide, and an organic acid transport (MAE) polypeptide as compared with its activity in an otherwise identical yeast lacking the modification.

271. The method of claim 270 wherein the at least one modification that increases organic acid production comprises the addition of a heterologous gene encoding a PYC polypeptide.

272. The method of claim 271 wherein the gene encoding a PYC polypeptide ncodes a PYC polypeptide having at least 95% identity to SEQ ID NO:67 (Y. lipolytica PYC1).

273. The method of claim 263 wherein the at least one modification that increases organic acid production comprises a genetic modification that increases activity of an MDH polypeptide.

274. The method of claim 273 wherein the at least one modification that increases organic acid production comprises the addition of a gene encoding a MDH3 polypeptide

275. The method of claim 263 wherein the at least one modification that increases organic acid production comprises the addition of a gene encoding a MTH1 polypeptide.

276. The method of claim 263 wherein the gene encoding an MTH polypeptide encodes MTH1ΔT.

277. The method of claim 263 wherein the genetic modification that increases organic production comprises a modification that decreases endogenous PYC activity.

278. The method of claim 263 wherein the at least one genetic modification that increases organic acid production comprises a genetic modification increases the activity of an organic acid transport polypeptide.

279. The method of claim 278 wherein the organic acid transport polypeptide is heterologous to the yeast.

280. The method of claim 263 wherein the genetically modified yeast comprises at least two modifications as compared with a parental yeast, the at least two modifications including: a first modification that reduces PDC polypeptide activity; and at least two additional modifications selected from the group consisting of a modification that increases pyruvate carboxylase (PYC) polypeptide activity, a modification that increases phosphoenolpyruvate carboxylase polypeptide activity (PPC activity), a modification that increases malate dehydrogenase (MDH) polypeptide activity, and a modification that increases (MAE) polypeptide activity.

281. The method of claim 263 further comprising isolating the organic acid produced.

282. The method of claim 263 wherein the genetically modified yeast is PDC1 pdc5 PDC6.

283. The method of claim 282 wherein the genetically modified yeast is PDC1 pdc5 PDC6 MTH1ΔT

284. The method of claim 283 wherein the genetically modified yeast is PDC1 pdc5 PDC6 MTH1ΔT and harbors a heterologous PYC.

285. A genetically modified yeast that is PDC1 pdc5 PDC6 and has at least one modification that increases organic acid production as compared with an otherwise identical yeast lacking the modification.

286. The genetically modified yeast of claim 285 the genetically modified yeast is PDC1 pdc5 PDC6 MTH1ΔT

287. The genetically modified yeast of claim 286 wherein the genetically modified yeast is PDC1 pdc5 PDC6 MTH1ΔT and harbors a heterologous PYC.
